(19) United States
(12) Patent Application Publication
(10) Pub. No.: US 2023/0059921 A1
(43) Pub. Date: Feb. 23, 2023

(54) METHODS AND COMPOSITIONS FOR DIAGNOSTICALLY-RESPONSIVE LIGAND-TARGETED DELIVERY OF THERAPEUTIC AGENTS

(71) Applicant: Ligandal, Inc., San Francisco, CA (US)

(72) Inventors: Andre Ronald Watson, San Francisco, CA (US); Shahab Chizari, San Francisco, CA (US); Ryan Spencer, San Francisco, CA (US); Christian Foster, Oakland, CA (US); Shuailiang Lin, San Francisco, CA (US); Sara Marie Peyrot, Berkeley, CA (US); Pranali Deshpande, San Francisco, CA (US); Matthew Dobbin, San Francisco, CA (US); William Connors, San Francisco, CA (US)

(21) Appl. No.: 17/453,336

(22) Filed: Nov. 2, 2021

Related U.S. Application Data
(63) Continuation of application No. PCT/US20/31188, filed on May 1, 2020.
(60) Provisional application No. 62/842,400, filed on May 2, 2019.

(51) Int. Cl. G01N 33/566; C40B 30/04

(52) U.S. Cl. CPC G01N 33/566; G01N 2500/10; C40B 30/04

(57) ABSTRACT

Provided are methods and compositions for the heterologous expression of a payload (e.g., DNA, RNA, protein) of interest in a target cell (e.g., cancer cell). In some cases payload delivery results in expression (e.g., by a cancer cell in vivo) of a secreted immune signal such as a cytokine, a plasma membrane-tethered affinity marker (thus resulting in an induced immune response), or a cytotoxic protein such as an apoptosis inducer (e.g., by a cancer cell in vivo). Payloads are delivered with a delivery vehicle and in some cases the delivery vehicle is a nanoparticle. In some cases a subject nanoparticle includes a targeting ligand for targeted delivery to a specific cell type/tissue type (e.g., a cancerous tissue/cell). In some embodiments, payload delivery is “personalized” in the sense that the delivery vehicle and/or payload can be designed based on patient-specific information.

### CROSS REFERENCE TO RELATED APPLICATIONS

This application is a continuation of International Patent Application No. PCT/US20/31188, filed May 1, 2020, which claims the benefit of U.S. Provisional Patent Application No. 62/842,400, filed May 2, 2019, both of which are incorporated herein by reference in their entirety.

### INTRODUCTION

Despite increasing advancements in gene sequencing, cell surface proteomics, and single-cell genomics, conversion of these data into personalized therapies has remained limited in the realm of cell-specific targeted delivery. Gene therapy and targeted nanomedicine approaches, in particular, have been in great need of improvements to cell-specific delivery technologies. Given the broadly varying expression profiles on various cells, tissues and organs within healthy and diseased physiology, there is a need for “diagnostically-responsive” medicine that can target a given cell/tissue/organ and present a precise set of instructions to that cell/tissue/organ.

One major hurdle for the successful treatment of cancer is that cancer manifests in many forms across all organ systems with each exhibiting diverse physiology. As such, response to treatment can be variable, and the effectiveness of some therapeutics is limited to specific phenotypes. Furthermore, genetic diseases and other degenerative conditions associated with aging morbidity pose a need for cell-specific targeting of genetic engineering tools.

Drug delivery to cancerous tissue can be accomplished via passive targeting due to leaky and irregular tumor vasculature with enhanced permeability and retention, which promotes the accumulation of macromolecules and nanoscale materials. However, this phenomena may not be consistent across patient populations. Furthermore, this phenomenon is not sufficient for achieving specific targeting of a given cell, tissue or organ type. Compositions and methods for efficiently targeting disease are provided in this disclosure, as well as for creating a diagnostically-responsive infrastructure for targeting a given cell/tissue/organ and delivering arbitrary gene editing or gene expressing instructions to those targets.

One difficulty in cancer immunotherapy stems from the fact that vaccination against cancers must bypass two forms of tolerance: central and peripheral. Central tolerance involves auto-reactive T cells being deleted whereas peripheral tolerance involves suppression of mature T cells through regulatory mechanisms and immune checkpoints. Such checkpoints can include the high expression of CTLA-4 or PD-1 receptors on tumor infiltrating lymphocytes. Recently, identifying and targeting tumor-specific antigens (neoantigens) which are only expressed in tumor cells has been of high interest as it can bypass central tolerance. However, the neoantigens can be patient specific and generally require either predictive modeling or patient genome sequencing. Thus, patient specific cancer vaccines are subject to significant time and cost. Efficient compositions and methods for patient-specific (diagnostically-responsive) treatments are provided in this disclosure, whereby a cancerous cell/tissue/organ (or another cell/tissue/organ being treated for disease) can be targeted for its specific receptor profile via an iterative nanoparticle development approach. The nanoparticles can furthermore deliver specific genetic instructions and be designed from bioresponsive materials that allow for additional cell-specific behaviors.

Oncolytic viruses (OVs) have been extensively studied as a cancer therapeutic as they selectively replicate and kill cancer cells without harming normal tissue. As an immunotherapy, OVs are used to tag, alert, and direct lymphocytes towards the tumors. Additionally, they have been used to transfect environment regulating cytokines such as GM-CSF into cancer cells to modulate the TME. However, the efficacy of these OVs to promote an immune response toward tumor cells is largely overshadowed by the immune response toward the OVs. Non-viral compositions and methods for efficiently targeting disease are provided in this disclosure.

### SUMMARY

Diagnostically-responsive medicine described herein can utilize a holistic nanoscale architecture coupled to a variety of cell-affinity-generating approaches for creating bioresponsive materials with many layers of precision in delivering a transient or permanent change in gene activity to a precisely-targeted cell, tissue or organ. Furthermore, an integrated robotics+software platform allows for rapid peptide synthesis, nanoparticle synthesis, and screening of formulations as part of a recursive machine learning approach for nanoparticle formulation optimization.

This approach goes beyond antibody-drug conjugates and traditional ligand-targeted medicine to create an end-to-end “diagnostically-responsive” medicine infrastructure featuring design, simulation, and synthesis suites driven by robotics, machine learning, biological characterization, nanomaterials characterization, and real-time data processing surrounding top-performing nanomedicine candidates as part of the detailed iterative improvement methodologies. Not only do these approaches offer combinatorial screening capabilities surrounding a comprehensive set of programmable matter, but each component of the nanomedicine/cell-targeting platform is designed to enhance specificity and afford patient-personalized therapeutic effect. These ligand-targeted solutions are readily manufacturing at cGMP grade through synthetic and/or recombinant means, to bolster industry adoption of cell-specific targeting technologies that are “user-specified” based on diagnostically-responsive traits and the payloads (e.g. CRISPR, DNA, mRNA, etc.) that are being delivered. Numerous formulations, embodiments, simulation and computation approaches, screening and synthesis approaches, methods, uses and variations thereof are detailed in the disclosure herein.

Using existing databases of cell, tissue and organ surface marker expression profiles, we show a novel approach for creating cell/tissue/organ-specific targeting technologies whereby a targeting ligand or array of targeting ligands designed to have specificity for a given surface marker profile are capable of shuttling a variety of payloads (e.g. gene therapies, RNPs, small molecules) to cells/tissues/organs bearing those surface markers. An integrative omics approach combines with novel nanomaterials and gene therapy/gene editing modalities such as CRISPR, DNA, and mRNA to allow for predictive targeting and amelioration of disease states, or synthetic biology characteristics (e.g. inserting chimeric antigen receptors into a particular immune subpopulation, or creating cell-specifically-expressed transmembrane motifs for subsequent affinity for an immunotherapy or gene therapy, and the like), in either healthy or diseased cell populations within specific cells/tissues/organs.

Design of targeted nanomedicine can allow for targeting specific cell types, including cancer neoantigens and known receptor profiles of target cells. Prior to this disclosure a diagnostically-responsive technology has not yet been deployed for rapidly tailoring cell-specific targeting technologies to a given patient's needs. Such a technology, as described in this disclosure, facilitates a future where patients see personalized medicine that is either permanent (e.g. CRISPR) or transient (e.g. mRNA), whereby targeted cells/tissues/organs are conferred disease resistance, genetic modifications, or immunomodulatory instructions.

Provided are methods and compositions for the heterologous expression of a payload (e.g., DNA, RNA, protein) of interest in a target cell (e.g., cancer cell, disease-causing cell/tissue/organ). In some cases payload delivery results in expression of a secreted protein, e.g., an immune signal such as a cytokine (e.g., by a cancer cell in vivo). In some cases payload delivery results in expression of a plasma membrane-tethered affinity marker (e.g., by cancer cells in vivo—thus resulting in an induced immune response). In some cases payload delivery results in expression of a cytotoxic protein such as an apoptosis inducer (e.g., by a cancer cell in vivo). In other cases, unknown cell types or cell types with known or acquired genomics/mRNA/proteomics data may be targeted “diagnostically-responsively” via a tailored cell targeting approach. In further cases, a combination of tumor surface marker engineering that is cell/tissue/organ-specific (e.g. under cancer-specific or cell-specific promoters) coupled to an immune engineering approach (e.g. causing antigen-presenting cells, γδ T cells, or other immune cells to hone in on the aforementioned cancer beacons).

Payloads are delivered with a delivery vehicle and in some cases the delivery vehicle is a nanoparticle. In some cases a subject nanoparticle for delivering payloads such as those discussed above includes a targeting ligand for targeted delivery to a specific cell type/tissue type (e.g., a cancerous tissue/cell).

In some embodiments, payload delivery and design of ligand-targeted, cell-specific nanomedicine is “personalized” in the sense that the delivery vehicle and/or payload can be designed based on patient-specific information—such embodiments are referred to herein as “personalized” or “diagnostically-responsive” methods. These diagnostically-responsive methods are facilitated by a nanomedicine infrastructure whereby design of optimal nanoparticles for a given payload, an appropriate cell-specific targeting strategy, and ultimately a cell-specific payload (e.g. promoter-driven expression, cell-specific Cas9 activity) are facilitated by a robotic, computationally-driven synthesis, screening and iteration approach. As such, in some cases a subject method involves diagnostically-responsive payload delivery (i.e., personalized payload delivery)—in such cases the delivery vehicle and/or the payload can be considered “personalized” where the “personalized” aspect relates to the ability to 1) identify ligand-receptor interactions based on native protein sequences (described herein) or alternative means (e.g. phage display, SELEX, etc.), 2) rapidly synthesize a cell-specific targeting ligand or combination of heteromultivalent cell-specific targeting ligands (e.g. through customized, ultra-high-speed robotic peptide synthesis described herein, or through other library generation techniques), 3) tethering these targeting ligands to a variety of nanoparticle chemistries (including electrostatic, lipidic and other embodiments), either through direct ligand condensation into a nanoparticle or upon the surface of a nanoparticle (or an alternative ligand-drug conjugate), 4) assaying for nanomaterials properties and biological effects (through a workflow described herein), 5) identifying top hit formulations via the properties of (4), and 6) iterating through the formulations, combinations of ligands and combinations/ratios of nanoparticle constituents (where applicable) through a software-driven approach (“recursive automation/machine learning”). The combination of this infrastructure with diagnostics data (e.g. receptor profiles, disease state of targeted cell, cell-specific promoter identification, target genes for expression/suppression/editing) and an underlying nanomaterials platform disclosed herein allows for customized, cell-specific targeting technologies to be developed in days or weeks vs. current industry approaches which take several months to years.

Such delivery systems offer flexibility and tailorability towards targeting patient-specific surface proteins and/or using selected promoters to drive expression of introduced sequences. For example, a promoter can be selected based on patient expression profiles. Thus, compositions and methods of this disclosure can be designed in a diagnostically responsive manner such that the composition/method can be tailored specifically for each patient. For example, once a tumor's unique characteristics are identified, a patient-specific and diagnostically-responsive nanomedicine (e.g., delivery vehicle that includes a payload) may be administered to the patient with or without the need for an autologous/allogeneic immunotherapy.

When compared to alternative delivery methods such as viruses, nanoparticles offer several key advantages. First, a lesser degree of immunogenicity may be achieved, and stealth properties may be incorporated in the design to prevent immune response, complement activation and subsequent clearance by the reticuloendothelial system. This immunogenicity may be further reduced by protein fragments (e.g. synthetic peptide sequences per the diagnostically-responsive workflow identified herein) being derived from native proteins when designing ligand-receptor pairings. Additionally, nanoparticles offer greater flexibility in the variety of payloads that may be encapsulated, as well as the potential for co-delivery of multiple payloads.

Further, nanoparticles composed of synthetic biopolymers such as peptides and nucleic acids may be easily tailored for different applications. This is particularly relevant to diagnostically responsive medicine.

The embodiments disclosed herein have broad application to drug delivery, immunotherapy, and oncology. Additionally, the embodiments herein present a universal approach for engineering cancer cells in a diagnostically responsive manner—e.g., to express markers that lead to adaptive immune learning, creating a novel cancer treatment that my augment autologous or allogeneic cell transplantation and engineered cell lines. The embodiments described herein can allow for improved tumor chemotaxis and prolonged adaptive immune learning.

### BRIEF DESCRIPTION OF THE DRAWINGS

This application contains at least one drawing executed in color. Copies of this application with color drawing(s) will be provided by the Office upon request and payment of the necessary fees.

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A depicts a schematic representation of example embodiments of a delivery package with a surface coat, sheddable layer, and core.

FIG. 1B depicts a schematic representation of example embodiments of a delivery package with a surface, interlayer, and core.

FIG. 2 depicts a schematic representation of an example embodiment of a delivery package (in the depicted case, one type of nanoparticle). In this case, the depicted nanoparticle is multi-layered, having a core (which includes a first payload) surrounded by a first sheddable layer, which is surrounded by an intermediate layer (which includes an additional payload), which is surrounded by a second sheddable layer, which is surface coated (i.e., includes an outer shell).

FIG. 3 (panels A-B) depicts schematic representations of example configurations of a targeting ligand of a surface coat of a subject nanoparticle. The delivery molecules depicted include a targeting ligand conjugated to an anchoring domain that is interacting electrostatically with a sheddable layer of a nanoparticle. Note that the targeting ligand can be conjugated at the N- or C-terminus (left of each panel), but can also be conjugated at an internal position (right of each panel). The molecules in panel A include a linker while those in panel B do not.

FIG. 4 (panels A-D) provides schematic drawings of an example embodiment of a delivery package (in the depicted case, example configurations of a subject delivery molecule). Note that the targeting ligand can be conjugated at the N- or C-terminus (left of each panel), but can also be conjugated at an internal position (right of each panel). The molecules in panels A and C include a linker while those of panels B and D do not. (panels A-B) delivery molecules that include a targeting ligand conjugated to a payload. (panels C-D) delivery molecules that include a targeting ligand conjugated to a charged polymer polypeptide domain that is condensed with a nucleic acid payload (and/or interacting, e.g., electrostatically, with a protein payload).

FIG. 5 provides non-limiting examples of nuclear localization signals (NLSs) that can be used (e.g., as part of a nanoparticle, e.g., as an NLS-containing peptide; as part of/conjugated to an NLS-containing peptide, an anionic polymer, a cationic polymer, and/or a cationic polypeptide; and the like). The figure is adapted from Kosugi et al., J Biol Chem. 2009 Jan 2; 284(1):478-85. (Class 1, top to bottom (SEQ ID NOs: 201-221); Class 2, top to bottom (SEQ ID NOs: 222-224); Class 4, top to bottom (SEQ ID NOs: 225-230); Class 3, top to bottom (SEQ ID NOs: 231-245); Class 5, top to bottom (SEQ ID NOs: 246-264)].

FIG. 6A depicts schematic representations of the mouse hematopoietic cell lineage, and markers that have been identified for various cells within the lineage.

FIG. 6B depicts schematic representations of the human hematopoietic cell lineage, and markers that have been identified for various cells within the lineage.

FIG. 7A depicts schematic representations of miRNA factors that can be used to influence cell differentiation and/or proliferation.

FIG. 7B depicts schematic representations of protein factors that can be used to influence cell differentiation and/or proliferation.

FIG. 8 depicts a schematic of example surface coats that can be used on the surface of a subject nanoparticle.

FIG. 9 depicts a schematic of one possible type of affinity marker, which a type of payload that can be delivered using a delivery vehicle as described herein.

FIG. 10A depicts the use of databases of mRNA sequencing or cell surface proteomics for individual cells, tissues and organs for generating lists of extracellular matrix proteins and ligands with which to mimic local environments when developing ligand-targeted gene or drug delivery systems. FPKM of 13 tissues: “We have used an integrative omics approach to study the spatial human proteome. Samples representing all major tissues and organs (n=44) in the human body have been analyzed based on 24,028 antibodies corresponding to 16,975 protein-encoding genes, complemented with RNA-sequencing data for 32 of the tissues.” (http://science.sciencemag.org/content/347/6220/1260419) The approach will be to utilize this and other databases, looking at extracellularly-presenting membrane proteins and comparing to known and acquired databases of protein sequences and crystal structures.

FIG. 10B depicts an algorithmic approach as further detailed in FIGS. 10C-10G, whereby mRNA sequencing and/or proteomics data is compared to evaluate the ratio of gene expression and/or protein expression in a target cell, tissue, or organ versus an off-target cell, tissue or organ. Below, inclusion criteria allow for sets of gene expression and/or protein expression databases to be compared in order to establish “selectivity indices” of a particular cell, tissue, or organ targeting approach. This informs subsequent approaches for designing, predictively modeling and/or synthesizing, and ultimately testing a given “diagnostically responsive” targeting approach. This modeling approach creates a unique targeting approach whereby multiple desired cell, tissue, and organ types may be deemed as acceptable targets (e.g. targeting lymph nodes and spleen are both useful for an immunoengineering approach targeting T cells) in addition to considerations of which cell types, including multiple cell types (e.g. T cells and B cells), should be targeted vs. avoided.

FIG. 10C depicts a database-driven approach to compiling surface markers. Inclusion criteria are shown for a given dataset and its top-expressed surface markers.

FIG. 10D depicts a database-driven approach to compiling surface markers. Exclusion criteria are shown for a given dataset and its top-expressed surface markers. Cell selectivity index allows for determining the specificity of a ligand-targeting approach (e.g. designed around target receptor profiles) for a given population of cells vs. another population.

FIG. 10E depicts a database-driven approach to compiling surface markers. Exclusion criteria are shown for a given dataset and its top-expressed surface markers. Tissue selectivity index allows for determining the specificity of a ligand-targeting approach (e.g. designed around target receptor profiles) for a given tissue vs. another population of cells and organs.

FIG. 10F depicts a database-driven approach to compiling surface markers. Exclusion criteria are shown for a given dataset and its top-expressed surface markers. Organ selectivity index allows for determining the specificity of a ligand-targeting approach (e.g. designed around target receptor profiles) for given cell type(s) AND organs vs. another population of cells and organs.

FIG. 10G depicts a basis for compiling databases of gene expression or protein expression data. Summed values of data, such as transcripts per million for RNAseq, may be used to compare various cell, tissue and organ expression profiles. While cell specificity index may be most useful for determining a targeting ligand approach within distinct cell subpopulations (as with many different kinds of hematological and immunological cells), tissue and organ specificity indices may be used to determine optimal strategies for achieving predicted biodistributions.

FIG. 11A depicts a lymph node case study and approach for applying sorting algorithms & cell specificity indices to determine top-expressed surface markers and concomitant ligands. Top-expressed surface markers are shown.

FIG. 11B depicts a lymph node case study and approach for applying sorting algorithm & cell specificity indices to top-expressed surface markers. Top-expressed surface markers in the target cell are shown with comparisons to the next-highest-expressing cell, tissue, or organ as determined through https://gtexportal.org/home/multiGeneQueryPage/. The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Spleen, Cells—EBV-transformed lymphocytes, Whole Blood, Small Intestine—Terminal Ileum, Testis, Liver, Lung, Minor Salivary Gland, Colon—Transverse, Skin—Sun Exposed (Lower leg), Skin—Not Sun Exposed (Suprapubic), Cells—Transformed fibroblasts, Muscle—Skeletal, Heart—Left Ventricle, Brain—Cerebellum, Brain—Cerebellar Hemisphere, Brain—Spinal cord (cervical c-1), Brain—Substantia nigra, Brain—Hypothalamus, Brain—Hippocampus, Brain—Amygdala, Brain—Frontal Cortex (BA9), Brain—Cortex, Brain—Putamen (basal ganglia), Brain—Anterior cingulate cortex (BA24), Brain—Nucleus accumbens (basal ganglia), Brain—Caudate (basal ganglia), Pituitary, Kidney—Cortex, Adipose—Visceral (Omentum), Thyroid, Artery—Aorta, Adipose—Subcutaneous, Breast—Mammary Tissue, Artery—Coronary, Ovary, Adrenal Gland, Pancreas, Heart—Atrial Appendage, Colon—Sigmoid, Artery—Tibial, Esophagus—Muscularis, Esophagus—Gastroesophageal Junction, Stomach, Esophagus—Mucosa, Bladder, Prostate, Fallopian Tube, Nerve—Tibial, Uterus, Cervix—Endocervix, Vagina, Cervix—Ectocervix.

FIG. 11C depicts an algorithmic scripting approach for establishing cell, tissue and organ specificity indices as well as top surface markers for specific targeting of a given cell, tissue, or organ.

FIG. 11D1 depicts an algorithmic comparison of top uniquely expressed in human naive CD8+ T cells. This particular dataset compares the top-expressed genes vs. the top uniquely expressed genes in the naive CD8+ T cell example, and compares to other immunological and blood cells. The y-axis of each graph shows transcripts per million.

FIG. 11D2 depicts an algorithmic comparison of top expressed genes in human naive CD8+ T cells. This particular dataset compares the top-expressed genes vs. the top uniquely expressed genes in the naive CD8+ T cell example, and compares to other immunological and blood cells. The y-axis of each graph shows transcripts per million.

FIG. 11E depicts an example of how a panel of genes expressed on Naive CD8+ T cells are compared in their expression profiles to a range of target organs. In this instance, whole blood, spleen, small intestine, and lung targeting present acceptable organs for achieving targeting of the given cell types given residence of T cells within each of the compartments. Additional targeting ligands may be utilized to further tune the targeting of one organ vs. another, while balancing specificity for a given cell type. The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Cells—EBV-transformed lymphocytes, Whole Blood, Spleen, Small Intestine—Terminal Ileum, Lung, Cells—Transformed fibroblasts, Brain—Cerebellum, Brain—Cerebellar Hemisphere, Brain—Nucleus accumbens (basal ganglia), Brain—Putamen (basal ganglia), Brain—Caudate (basal ganglia), Muscle—Skeletal, Heart—Left Ventricle, Pancreas, Brain—Substantia nigra, Brain—Hypothalamas, Brain—Hippocampus, Brain—Amygdala, Brain—Cortex, Brain—Frontal Cortex (BA9), Brain—Anterior cingulate cortex (BA24), Pituitary, Brain—Spincal cord (cervical c-1), Testis, Adrenal Gland, Skin—Sun Exposed (Lower leg), Skin—Not Sun Exposed (Suprapubic), Ovary, Artery—Tibial, Heart—Atrial Appendage, Liver, Kidney—Cortex, Colon—Sigmoid, Esophagus—Muscularis, Esophagus—Castroesophageal Junction, Bladder, Adipose—Visceral (Omentum), Nerve—Tibial, Aretery—Aorta, Adipose—Subcutaneous, Minor Salivary Gland, Cervix—Endocervix, Breast—Mammary Tissue, Artery—Coronary, Uterus, Esophagus—Mucosa, Stomach, Colon—Transverse, Thyroid, Fallopian Tube, Cervix—Ectocervix, Vagina, Prostate.

FIG. 11F depicts results of an algorithmic approach to identifying cell and organ specificity indices (y-axises of middle and top graphs) of top expressed genes in Naive CD8+ T cells. The bottom shows transcripts per million (TPM) of each overexpressed gene. A given top expressed gene's mRNA expression (transcripts per million) is divided by the expression within the next-highest-expressing cell or organ to determine cell and organ specificity indices. These quantitative numbers give a more precise unique receptor profile than merely ranking top-expressed genes, as it factors in relative gene expression to other cells (top) and organs (middle). Depending on whether cell or organ specificity is desired, either a cell specificity or organ specificity index may be used.

FIG. 11G depicts a skeletal muscle membrane protein case study and approach for applying sorting algorithms & cell specificity indices to determine top-expressed surface markers and concomitant ligands. Top-expressed surface markers are shown.

FIG. 11H compares top skeletal muscle membrane protein expression profiles (transcripts per million) to other tissues and organs (continuation of FIG. 11G). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Muscle—Skeletal, Heart—Left Ventricle, Heart—Atrial Appendage, Testis, Brain—Cerebellum, Brain—Cerebellar Hemisphere, Pituitary, Brain—Spinal cord (cervical c-1), Brain—Anterior cingulate cortex (BA24), Brain—Frontal Cortex (BA9), Brain—Cortex, Brain—Nucleus accumbens (basal ganglia), Brain—Putamen (basal ganglia), Brain—Caudate (basal ganglia), Brain—Substantia nigra, Brain—Hypothalamus, Brain—Hippocampus, Brain—Amygdala, Liver, Cells—EBV-transformed lymphocytes, Whole Blood, Pancreas, Adrenal Gland, Nerve—Tibial, Prostate, Bladder, Thyroid, Kidney—Cortex, Stomach, Cells—Transformed fibroblasts, Spleen, Ovary, Skin—Sun Exposed (Lower leg), Skin—Not Sun Exposed (Suprapubic), Adipose—Subcutaneous, Breast—Mammary Tissue, Adipose—Visceral (Omentum), Fallopian Tube, Artery—Tibial, Artery—Coronary, Minor Salivary Gland, Esophagus—Mucosa, Colon—Sigmoid, Artery—Aorta, Esophagus—Muscularis, Esophagus—Gastroesophageal Junction, Small Intestine—Terminal Ileum, Lung, Vagina, Colon—Transverse, Uterus, Cervix—Endocervix, Cervix—Ectocervix.

FIG. 11I depicts a bone marrow membrane protein case study and approach for applying sorting algorithms & cell specificity indices to determine top-expressed surface markers and concomitant ligands. Top-expressed surface markers are shown.

FIG. 11J compares top bone marrow membrane protein expression profiles (transcripts per million) to other tissues and organs (continuation of FIG. 11I). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Spleen, Cells—EBV-transformed lymphocytes, Small Intestine—Terminal Ileum, Whole Blood, Lung, Testis, Brain—Cerebellumn, Brain—Cerebellar Hemisphere, Brain—Spinal cord (cervical c-1), Brain—Putamen (basal ganglia), Brain—Cortex, Brain—Nucleus accumbens (basal ganglia), Brain—Caudate (basal ganglia), Brain—Frontal Cortex (BA9), Brain—Cortex, Brain—Anterior cingulate cortex (BA24), Brain—Substantia nigra, Brain—Hypothalamas, Brain—Hippocampus, Brain—Amygdala, Liver, Skin—Sun Exposed (Lower leg), Skin—Not Sun Exposed (Suprapubic), Colon—Transverse, Vagina, Minor Salivary Gland, Esophagus—Mucosa, Ovary, Pituitary, Adrenal Gland, Kidney—Cortex, Nerve—Tibial, Thyroid, Artery—Coronary, Artery—Aorta, Adipose—Visceral (Omentum), Breast—Mammary Tissue, Adipose—Subcutaneous, Cells—Transformed fibroblasts, Pancreas, Muscle—Skeletal, Heart—Left Ventricle, Prostate, Stomach, Fallopian Tube, Heart—Atrial Appendage, Artery—Tibial, Esophagus—Muscularis, Esophagus—Gastroesophageal Junction, Colon—Sigmoid, Bladder, Cervix—Endocervix, Utuerus, Cervix—Ectocervix.

FIG. 11K compares top skeletal muscle membrane protein expression profiles (transcripts per million) to other tissues and organs (continuation of FIGS. 11I-11J). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Spleen, Whole Blood, Lung, Cells—EBV-transformed lymphocites, Vagina, Esophagus—Mucosa, Skin—Sun Exposed (Lower leg), Skin—Not Sun Exposed (Suprapubic), Brain—Cerebellum, Brain—Cerebellar Hemisphere, Brain—Anterior cingulate cortex (BA24), Brain—Frontal Cortex (BA9), Brain—Cortex, Brain—Caudate (basal ganglia), Brain—Substantia nigra, Brain—Hypothalamus, Brain—Hippocampus, Brain—Amygdala, Cells—Transformed fibroblasts, Pituitary, Small Intestine—Terminal Ileum, Colon—Transverse, Testis, Brain—Spinal cord (cervical c-1), Ovary, Muscle—Skeletal, Colon—Sigmoid, Esophagus—Muscularis, Esophagus—Gastroesophageal Junction, Minor Salivary Gland, Pancreas, Heart—Left Ventricle, Artery—Aorta, Liver, Heart—Atrial Appendage, Kidney—Cortex, Artery—Tibial, Adrenal Gland, Thyroid, Bladder, Artery—Coronary, Adipose—Visceral (Omentum), Fallopian Tube, Breast—Mammary Tissue, Adipose—Subcutaneous, Stomach, Nerve—Tibial, Uterus, Cervix—Endocervix, Prostate, Cervix—Ectocervix.

FIG. 11L depicts a neural (cerebral cortex) membrane protein case study and approach for applying sorting algorithms & cell specificity indices to determine top-expressed surface markers and concomitant ligands. Top-expressed surface markers are shown.

FIG. 11M depicts top-expressed neural membrane proteins.

FIG. 11N depicts a comparison of brain enriched proteins to other organs. 419 genes are uniquely overexpressed in the brain. Of these 419 genes, 140 are potentially relevant surface markers for subsequent ligand targeting as determined by algorithmic subclassifications and selectivity indices.

FIG. 11O compares top-expressed neural membrane protein expression profiles (transcripts per million) to other tissues and organs (continuation of FIGS. 11L-11N). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Testis, Pituitary, Brain—Cerebellum, Brain—Cerebellar Hemisphere, Brain—Substantia nigra, Brain—Spinal cord (cervical c-1), Brain—Hypothalamus, Brain—Nucleus accumbens (basal ganglia), Brain—Putamen (basal ganglia), Brain—Caudate (basal ganglia), Brain—Hippocampus, Brain—Amygdala, Brain—Anterior cingulate cortex (BA24), Brain—Frontal Cortex (BA9), Brain—Cortex, Adrenal Gland, Prostate, Nerve—Tibial, Stomach, Heart—Left Ventricle, Heart—Atrial Appendage, Lung, Skin—Sun Exposed (Lower leg), Skin—Not Sun Exposed (Suprapubis), Artery—Aorta, Artery—Tibial, Artery—Coronary, Thyroid, Muscle—Skeletal, Colon—Sigmoid, Small Intestine—Terminal Ileum, Colon—Transverse, Esophagus—Muscularis, Esophagus—Gastroesophageal Junction, Minor Salivary Gland, Adipose—Visceral (Omentum), Breast—Mammary Tissue, Adipose—Subcutaneous, Pancreas, Spleen, Cells—Transformed fibroblasts, Liver, Whole Blood, Esophagus—Mucosa, Cells—EBV-transformed lymphocytes, Ovary, Kidney—Cortex, Fallopian Tube, Bladder, Uterus, Cervix—Endocervix, Vagina, Cervix—Ectocervix.

FIG. 11P compares top-expressed neural membrane protein expression profiles (transcripts per million) to other tissues and organs (continuation of FIGS. 11M-11O). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Testis, Pituitary, Brain—Cerebellum, Brain—Cerebellar Hemisphere, Brain—Hypothalamus, Brain—Anterior cingulate cortex (BA24), Brain—Frontal Cortex (BA9), Brain—Cortex, Brain—Spinal cord (cervical c-1), Brain—Substantia nigra, Brain—Hippocampus, Brain—Amygdala, Brain—Nucleus accumbens (basal ganglia), Brain—Putamen (basal ganglia), Brain—Caudate (basal ganglia), Adrenal Gland, Muscle—Skeletal, Heart—Left Ventricle, Heart—Atrial Appendage, Cells—Transformed fibroblasts, Liver, Whole Blood, Spleen, Cells—EBV-transformed lymphocytes, Pancreas, Kidney—Cortex, Nerve—Tibial, Small Intestine—Terminal Ileum, Thyroid, Vagina, Esophagus—Mucosa, Skin—Sun Exposed (Lower leg), Skin—Not Sun Exposed (Suprapubic), Prostate, Minor Salivary Gland, Stomach, Bladder, Colon—Transverse, Colon—Sigmoid, Esophagus—Muscularis, Esophagus—Gastroesophageal Junction, Ovary, Adipose—Subcutaneous, Breast—Mammary Tissue, Adipose—Visceral (Omentum), Lung, Artery—Aorta, Artery—Tibial, Artery—Coronary, Uterus, Fallopian Tube, Cervix—Endocervix, Cervix—Ectocervix.

FIG. 11Q compares top-expressed neural membrane protein expression profiles (transcripts per million) to other tissues and organs (continuation of FIGS. 11M-11P). The classifier subcategorizes the membrane proteins to look at relative comparisons for the top-expressed membrane proteins as seen on the vertical axis lists of genes. The horizonatal axis is sorted from left to right according to the most similar gene expression to the least similar gene expression by sample: Brain—Cerebellum, Brain—Cerebellar Hemisphere, Brain—Spinal cord (cervical c-1), Brain—Nucleus accumbens (basal ganglia), Brain—Putamen (basal ganglia), Brain—Hypothalamus, Brain—Hippocampus, Brain—Amygdala, Brain—Anterior cingulate cortex (BA24), Brain—Frontal Cortex (BA9), Brain—Cortex, Testis, Pituitary, Muscle—Skeletal, Whole Blood, Vagina, Esophagus—Mucosa, Skin—Sun Exposed (Lower leg), Skin—Not Sun Exposed (Suprapubic), Nerve—Tibial, Thyroid, Spleen, Kidney—Cortex, Adrenal Gland, Cells—Transformed fibroblasts, Liver, Small Intestine—Terminal Ileum, Cells—EBV-transformed lymphocytes, Stomach, Pancreas, Lung, Heart—Left Ventricle, Heart—Atrial Appendage, Artery—Coronary, Artery—Tibial, Artery—Aorta, Ovary, Prostate, Fallopian Tube, Uterus, Cervix—Endocervix, Cervix—Ectocervix, Minor Salivary Gland, Adipose—Subcutaneous, Breast—Mammary Tissue, Adipose—Visceral (Omentum), Bladder, Colon—Transverse, Colon—Sigmoid, Esophagus—Muscularis, Esophagus—Gastroesophageal Junction.

FIG. 11R depicts schematics of differential surface marker expression between different cell types, shown for lymph nodes vs. the next-highest-expressing cell type or organ that is not relevant for immunoengineering. Shown are exemplary crystal structures of the top-expressed genes.

FIG. 11S1 depicts a machine learning based approach for determining unique surface markers in a mixed cell population, allowing for improved classification of cell specificity indices. In this example, hematopoietic stem cells and their progenitors are shown. tSNE, principle component analysis (PCA) and similar unsupervised learning techniques may be used to determine initial sets of surface markers corresponding to a particular cell population subtype.

FIG. 11S2 depicts an enlarged view of the top nine plots of FIG. 11S1.

FIG. 11S3 depicts an enlarged view of the bottom six plots of FIG. 11S1.

FIG. 12A depicts a table showing various ligand approaches that may be used corresponding to top-expressed surface markers.

FIG. 12B depicts a schematic of de novo peptide/peptoid ligand design. An in silico (computational) screening approach is shown. This approach may be used with a variety of ligands and classes of molecules where receptor-ligand pairings may be simulated or modeled. This figure also includes embodiments where ligand molecules that bind receptors are not peptide based (e.g. small molecules, neurotransmitters, cholesterol, etc.). Phage display, SELEX, and other peptide/aptamer discovery approaches may also be utilized, wherein the ligands are subsequently paired to a linker and/or anchor domain.

FIG. 12C depicts a schematic detailing assembly of variable ligands, anchors, linkers, and/or other domains combinatorially. After surface markers are identified and the binding domains of similar structures of protein-receptor interactions (based on approaches described elsewhere throughout the patent and shown here) will be used to create a new peptide ligand (or alternative ligand) with receptor specificity. It will then be paired combinatorially with various linker (e.g. GGGGSGGGGS) and anchor (e.g. histone tail peptide, 9R, lysines, etc.) domains to create optimal nanoparticles. Anchor, linker and ligand combinations with optimal physicochemical and biological properties for a given payload or delivery application are further iterated around with changes to amino acid isomeric composition, hydrophobicity, charge, sequence, and functional domains as detailed elsewhere. In some embodiments, a direct chemical conjugation of a payload may be used with a ligand and/or linker pairing. The combinatorial library technique shown here allows for screening many linker and anchor lengths, sequences, and properties, while allowing for new ligands to modularly reconfigured on existing anchor-linker libraries.

FIG. 12D depicts examples of binding substrates for anchor-linker-ligands or linker-ligands, variable anchor domains, coupling chemistries, and linker domains.

FIG. 13A depicts examples of how various laboratory equipment is utilized to generate novel peptide sequences, novel nanoparticle variants, and quantitative values for nanoparticle size, charge, transfection efficiency, gene expression/editing, and other data useful for physicochemical/biological characterization of nanoparticle performance. The output data is fed back into a formulator approach for improving the nanoparticles recursively.

FIG. 13B depicts examples of how physicochemical nanoparticle data and biological data can be outputted into databases and processed as training data to lead to improvements in formulations via supervised (regression, classification) and unsupervised learning (clustering, collaborative filtering, reinforcement learning, tSNE, PCA) approaches. Top performing nanoparticle candidates can be recursively optimized.

FIG. 13C depicts examples of degrees of freedom utilized by robotic fluid handling and/or microfluidic approaches in order to optimize nanoparticle performance and physicochemical properties. 12 degrees of freedom are shown, which can be studied in ranges.

FIG. 13D depicts how automation and high-throughput nanoparticle synthesis can be used to separately optimize nanoparticle core designs and nanoparticle surface chemistry/ligand presentation designs. Examples are shown whereby 10,000 core formulations are compared to 10,000 ligands in order to establish an optimal nanoparticle. In other cases, 10 ligands are used with ˜100 cores embodiments or 1000 core embodiments, and each iteration leads to a multiplier effect in terms of the combinatorial state-space evaluated.

FIG. 13E depicts a nanoparticle formulator application front-end interface, which is converted to robotic fluid handling code. In this diagram, valence represents how many ligands/species will be present in the given formulation, while Pos-Neg Start shows the cationic amino acid amine ratio to the anionic amino acid carboxylate and nucleotide phosphate sequences [N/(P+C)] starting point, and “End” shows the final ratio. In this example, +/−ratios of 3 are studied.

FIG. 13F depicts the next prompt page of the formulator app interface, which allows for selection of relevant targeting ligands for a given set of payloads, and establishing molar fractions of each species per formulation.

FIG. 13G depicts the next prompt page of formulator app interface allowing for input of concentration (w/v) of each payload, polymer, and/or ligand, as well as associated transfection volumes.

FIG. 13H depicts another example FIG. 13F, whereby the formulator app interface allows for co-delivering multiple payloads (in this screenshot, a NLS-Cas9-EGFP Cas9 RNP targeting TRAC, and a dsDNA inserting mTagRFP2 into the IRAC locus. The formulator app accounts for the charge contributions of each payload, and designs the associated charge ratios of cationic and anionic polymers/polypeptides appropriately.

FIG. 13I depicts Instructions for robotic fluid handling mediated nanoparticle synthesis generated by the formulator app. Shown are 57 nanoparticle variants. Top row indicates well number, well locations, C:P (carboxylate to phosphate) ratio, P:N (positive to negative ratio), volume of water (uL), volume of buffer (pH 5.5 or pH 7.4 HEPES), volume of Cas9-EGFP RNP, and the volume of each of the three displayed targeting ligands or cationic polymers (CD3, CD28, CD3) as well as poly(glutamic acid) (PLE100:PDE100 in a 1:1 ratio). The total volume of each synthesis is 60 uL, allowing for transfection in triplicate in 10 uL/well doses in 96-well plates.

FIG. 13J depicts a schematic representation of input data (cell surface marker overexpression, compartment/cell/tissue/organ-specific proteolytic enzymes, and cell-specific promoters) leading to design of “diagnostically-responsive” payloads and ligands. These payloads and ligands are subsequently combined with a variety of biopolymers and/or nanoparticle components through automated liquid handling approaches, which are then assessed for biological and physicochemical performance through metrics described elsewhere.

FIG. 14A depicts examples of a variety of ligands, stealth motifs, and payloads that are screened in the process of developing ideal delivery systems. In this example, Possible Payload A includes plasmids or minicircle DNA. Possible Payload B includes dsDNA fragments, ssODNs, mRNAs, miRNAs, siRNA, or other charged linear DNAs/RNAs. Possible Payload C includes a protein or colloidally stable nanoparticle surface, such as CRISPR RNPs, other proteins, metallic or theranostic particle templates, and the like.

FIG. 14B depicts a schematic representation of affinity marker platform, whereby variable transmembrane domains (with optional intracellular signaling domains), linker domains, and functional domains may be used. These domains may each serve a variety of purposes, may be derived from a range of human proteins or synthetic exogenous proteins, and ultimately serve to produce “specific anchors” on a given cell/tissue/organ/cancer type that can subsequently be targeted in a variety of ways, including through immunoengineering approaches and subsequent dosing by nanoparticles with affinity for the functional domains (“functional domain” is used interchangeably here with “affinity marker”).

FIG. 14C depicts a schematic representation of how exemplary particles in 14A may be used to mark a cell for subsequent immunogenic response.

FIG. 14D depicts a schematic representation of how exemplary particles in 14A and cells in 14B may be used to trigger T-cell or other specific immune cell responses (e.g. through paired TCR/chimeric antigen receptor targeting of the expressed affinity marker). In this example, the cell killing response of cells/tissues/organs/cancers expressing affinity markers may be mediated in a number of ways.

FIG. 14E depicts a schematic representation of how affinity marker expressing cells may be used with CAR-T cells possessing specificity for the expressed affinity marker.

FIG. 14F depicts a schematic representation whereby two or more different particles in 14A can be delivered to 1) a target cell (e.g. an immune cell, stem cell, or other circulating cell) to express a chimeric receptor that is specific to an affinity marker and 2) a diseased cell (e.g. a cancerous cell, senescent cell, and the like) to express a corresponding affinity marker. Subsequently, the two cells would gain affinity for each other.

FIG. 15A1 depicts synthesis results of bulk mixing histone-derived, cysteine-substituted amino acid sequences in various pH conditions and with variable crosslinking time, which yielded an optimal condensation profile with cores made in 30 mM pH 5.5 HEPES. These nanoparticles were used to deliver CRISPR Cas9 RNPs. Inclusion of serum in these particle formulations led to enhanced particle condensation as assessed via SYBR inclusion assay. RNP (5 ng/uL) control fluorescent values (+ and −serum) are shown for baseline SYBR assay values prior to nanoparticle condensation.

FIG. 15A2 depicts the particle sizes corresponding to the FIG. 15A1 embodiment.

FIG. 15A3 depicts the particle sizes distribution corresponding to the FIG. 15A1 embodiment.

FIG. 15B1 depicts orders of addition studies of poly(glutamic acid) and cysteine-modified histone fragments with CRISPR Cas9 RNPs, whereby particle size and formation behaviors were not shown to be different between the two orders of addition when the synthesis was performed via microfluidic devices, and microfluidic mixing led to enhanced particle sizes with uniform size peaks versus bulk synthesis approaches (FIG. 15A1-3). Adding PLE before H2B or H2B before PLE in the microfluidic approach did not impact core particle formation. Inclusion of serum in these particle formulations led to enhanced particle condensation as assessed via SYBR inclusion assay.

FIG. 15B2 depicts the particle sizes corresponding to the FIG. 15B1 embodiment.

FIG. 15B3 depicts the particle sizes distribution corresponding to the FIG. 15B1 embodiment.

FIG. 15C1 depicts nanoparticle cores prepared in FIG. 15B1-3 were subsequently patterned in a variety of electrostatic surface ligands, and the SYBR inclusion/exclusion assay values were measured for each formulation with and without serum inclusion. Particles synthesized with a 1 h crosslinking time demonstrated less stability than particles that had ligands immediately added to them prior to crosslinking, as inferred by the increase in SYBR fluorescence values in the 1 h crosslinked cores. This is perhaps due to serum dissociating the ligands and destabilizing the particles with 1 h of crosslinking, which led to a less stable colloid. Alternatively, ligand inclusion at an earlier stage may form a more stable suspension. Each ligand coating in these examples where a 0 h crosslinking time was utilized prior to ligand decoration demonstrated excellent SYBR fluorescence values with serum inclusion, and particle sizes remained stable with the RNP-H2B; RNP-H2B-PLE; Core—CD28(80), CD28(86), CD3e, IL2R; Core—CD28(80), CD28(86), CD3e, IL2R; and other heteromultivalent variants. Particle sizes were also demonstrably uniform for a variety of surface coats. See FIG. 17D for expanded datasets on particle size and zeta potential.

FIG. 15C2 depicts the particle sizes corresponding to the FIG. 15C1 embodiment.

FIG. 15C3 depicts the particle sizes distribution corresponding to the FIG. 15C1 embodiment.

FIG. 15D1 depicts expanded datasets for FIG. 15C1-3 for particle size following microfluidic core particle synthesis and subsequent layering with ligands. The size and zeta potential for each formulation, with cores that were crosslinked for either 0 h or 1 h, is shown. Size and zeta potential is compared with and without serum.

FIG. 15D2 depicts the zeta potential corresponding to the FIG. 15D1 embodiment.

FIG. 15E1 depicts extended SYBR fluorescence assays (24 h) without serum a for CRISPR RNP formulations in FIGS. 15A1-15D3.

FIG. 15E2 depicts the data corresponding to the FIG. 15E1 embodiment with serum.

FIG. 15F depicts SYBR fluorescent assay (mRNA inclusion curve) results whereby the methods and techniques used in FIGS. 15A1-15E3 were utilized to condense EGFP mRNA into nanoparticle cores. A variety of ratios of histone fragments, PLR10, and PLE20 were utilized. Shown is the charge ratio of poly(glutamic acid) carboxylates to nucleic acid phosphates and the charge ratio of histone or PLR10 amines to net negative (phosphate+carboxylate) groups.

FIG. 15G depicts SYBR fluorescent assay (mRNA inclusion curve) results whereby the methods and techniques used in FIGS. 15A1-15E3 were utilized to condense EGFP mRNA into nanoparticle cores. A variety of ratios of histone fragments, PLR10, and PLE20 were utilized. Shown is the charge ratio of poly(glutamic acid) carboxylates to nucleic acid phosphates and the charge ratio of histone or PLR10 amines to net negative (phosphate+carboxylate) groups.

FIG. 15H depicts SYBR fluorescent assay (mRNA inclusion curve) results whereby the methods and techniques used in FIGS. 15A1-15E3 were utilized to condense EGFP mRNA into nanoparticle cores. A variety of ratios of histone fragments, PLR10, and PLE20 were utilized. Shown is the charge ratio of poly(glutamic acid) carboxylates to nucleic acid phosphates and the charge ratio of histone or PLR10 amines to net negative (phosphate+carboxylate) groups.

FIG. 16A depicts an initial heteromultivalent screen of EGFP-Cas9 delivery was performed (FIGS. 8B1-8U3) prior to subsequent experiments (see FIGS. 12A-12C for illustrative examples) which assessed editing for an expanded set of nanoparticle cores, targeting ligand densities, and the like. In these experiments, EGFP-Cas9 nanoparticles were studied in human primary T cells and PBMC. EGFP uptake was quantitated 24 h post-transfection.

FIG. 16B1 depicts an untreated control for Cas9 uptake in T cells and PBMC. Negative Control+/−1%=noise Used as the basis to set gates for positive Cas9 signal.

FIG. 16B2 depicts the T cell data corresponding to FIG. 16B1.

FIG. 16B3 depicts the PBMC data corresponding to FIG. 16B1.

FIG. 16C1 depicts core nanoparticle only Cas9 uptake in T cells and PBMC. Does not contain targeting moieties.

FIG. 16C2 depicts the T cell data corresponding to FIG. 16C1.

FIG. 16C3 depicts the PBMC data corresponding to FIG. 16C1.

FIG. 16D1 depicts core nanoparticle+PLR10 cell penetrating peptide Cas9 uptake in T cells and PBMC. General cell surface proteoglycan targeting. Does not confer cell specificity

FIG. 16D2 depicts the T cell data corresponding to FIG. 16D1.

FIG. 16D3 depicts the PBMC data corresponding to FIG. 16D1.

FIG. 16E1 depicts core nanoparticle+CD3epsilon ligand Cas9 uptake in T cells and PBMC.

Monovalent surface targeting CD3. Broad T cell/Thymocyte specificity.

FIG. 16E2 depicts the T cell data corresponding to FIG. 16E1.

FIG. 16E3 depicts the PBMC data corresponding to FIG. 16E1.

FIG. 16F1 depicts core nanoparticle+CD8 ligand Cas9 uptake in T cells and PBMC. Monovalent surface targeting CD8. Results in significant uptake in T-cells and PBMCs.

FIG. 16F2 depicts the T cell data corresponding to FIG. 16F1.

FIG. 16F3 depicts the PBMC data corresponding to FIG. 16F1.

FIG. 16G1 depicts core nanoparticle only+CD80-derived CD28-targeting ligand Cas9 uptake in T cells and PBMC. Targets CD28, a T-cell marker. Ligand mimics CD80 on antigen-presenting cells. Modest uptake in T-cells.

FIG. 16G2 depicts the T cell data corresponding to FIG. 16G1.

FIG. 16G3 depicts the PBMC data corresponding to FIG. 16G1.

FIG. 16H1 depicts core nanoparticle+CD86-derived CD28-targeting ligand Cas9 uptake in T cells and PBMC. Targets CD28, a T-cell marker. Ligand mimics CD86 on antigen-presenting cells. No uptake in T-cells.

FIG. 16H2 depicts the T cell data corresponding to FIG. 16H1.

FIG. 16H3 depicts the PBMC data corresponding to FIG. 16H1.

FIG. 1611 depicts core nanoparticle+IL2-derived IL2R-targeting ligand Cas9 uptake in T cells and PBMC. Monovalent surface targeting IL2R. Modest uptake in T-cells.

FIG. 1612 depicts the T cell data corresponding to FIG. 1611.

FIG. 1613 depicts the PBMC data corresponding to FIG. 1611.

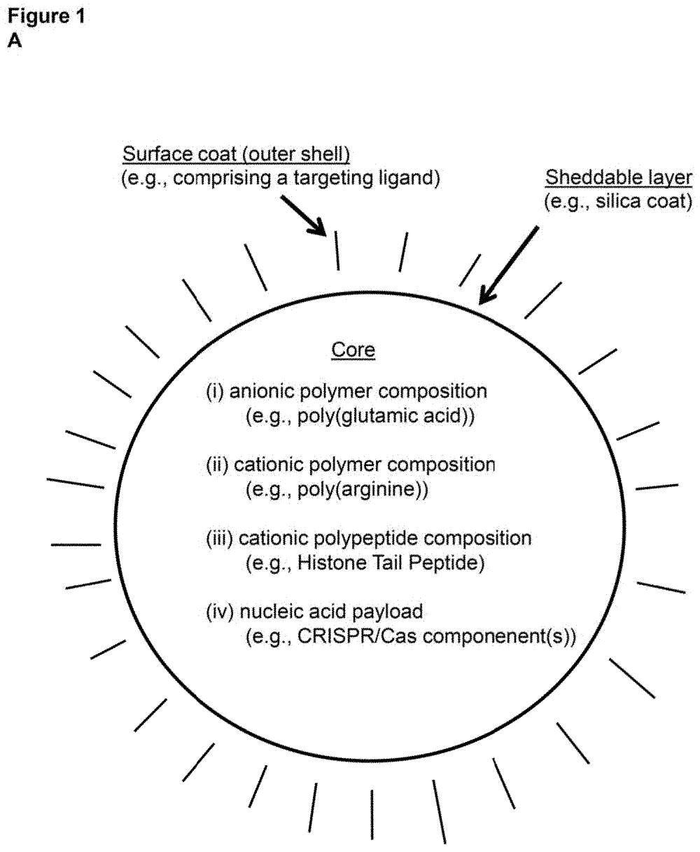

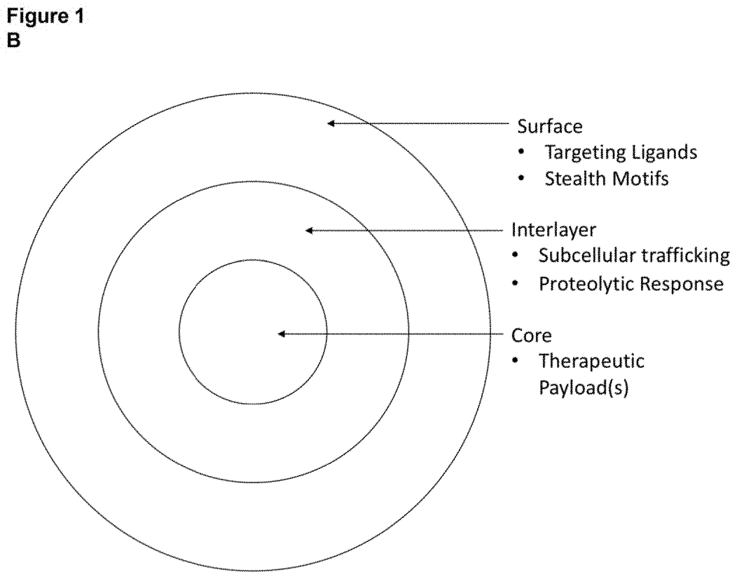

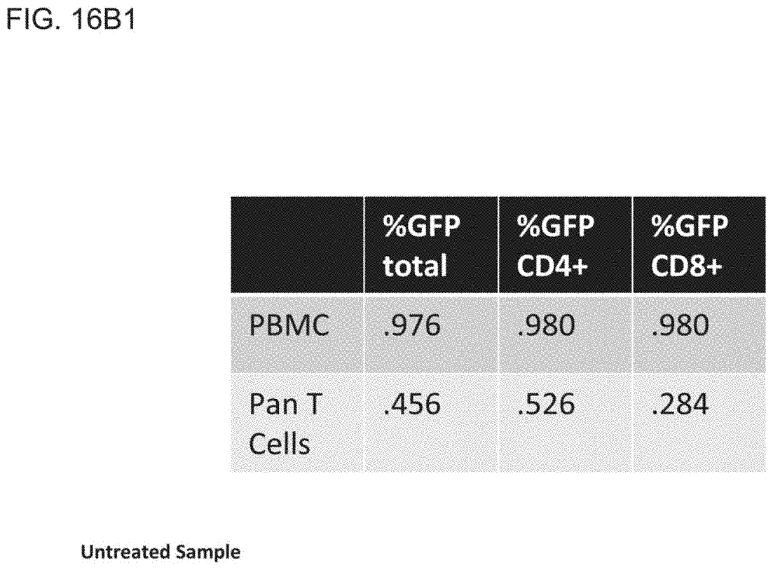

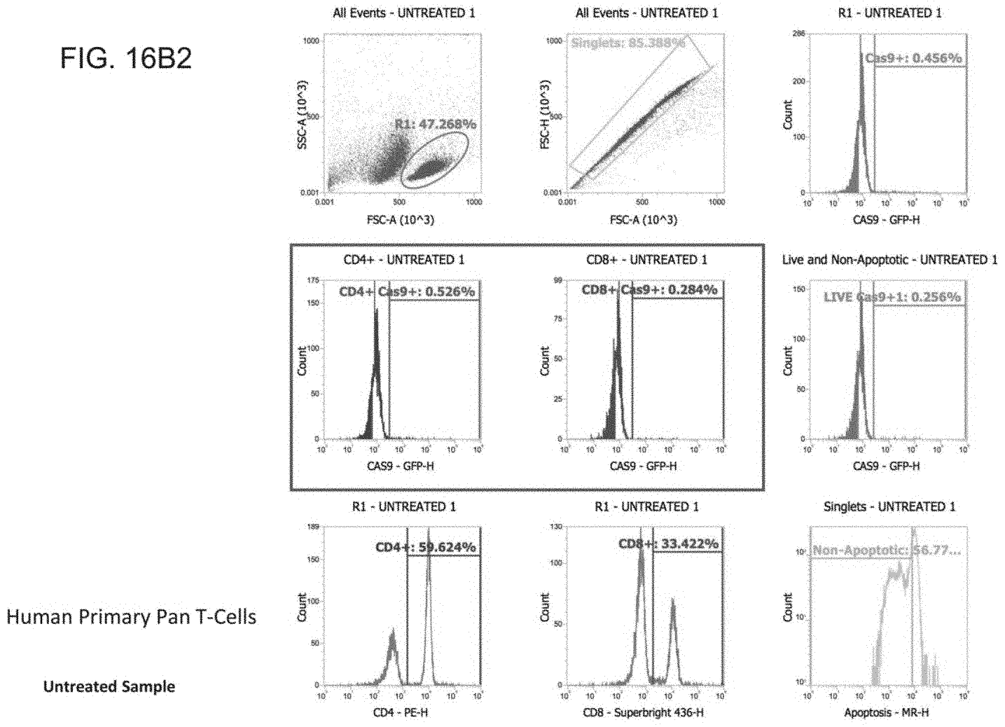

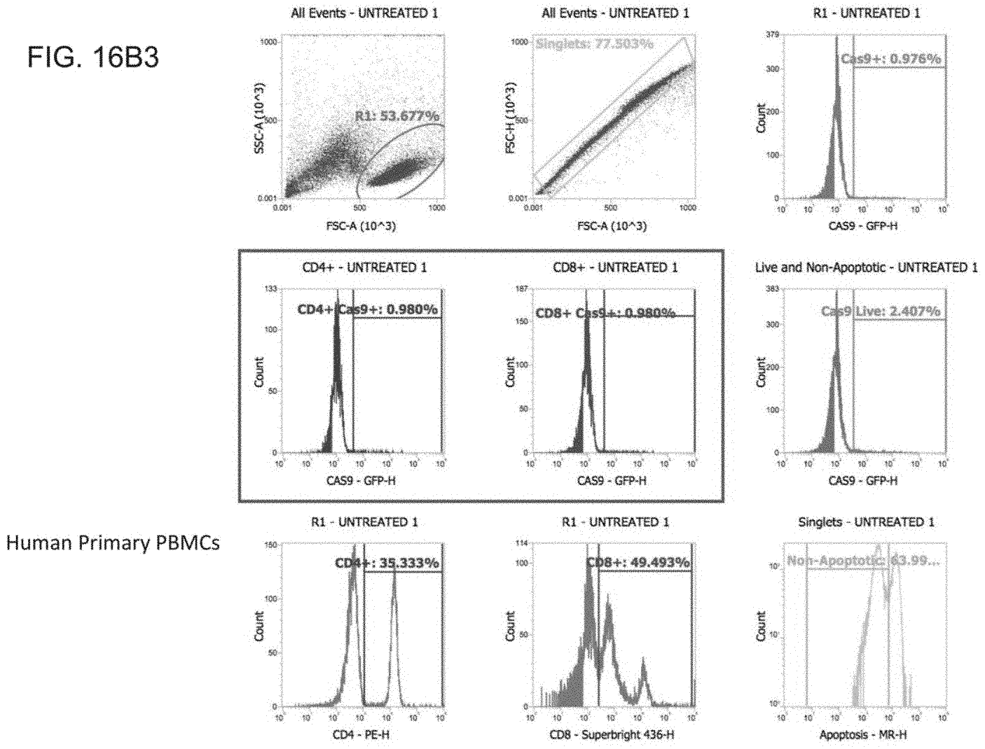

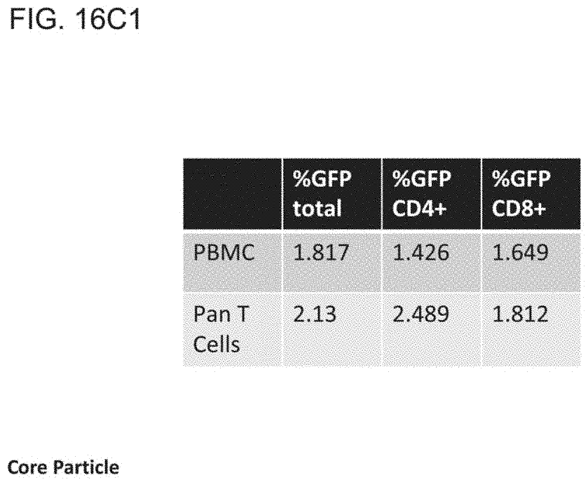

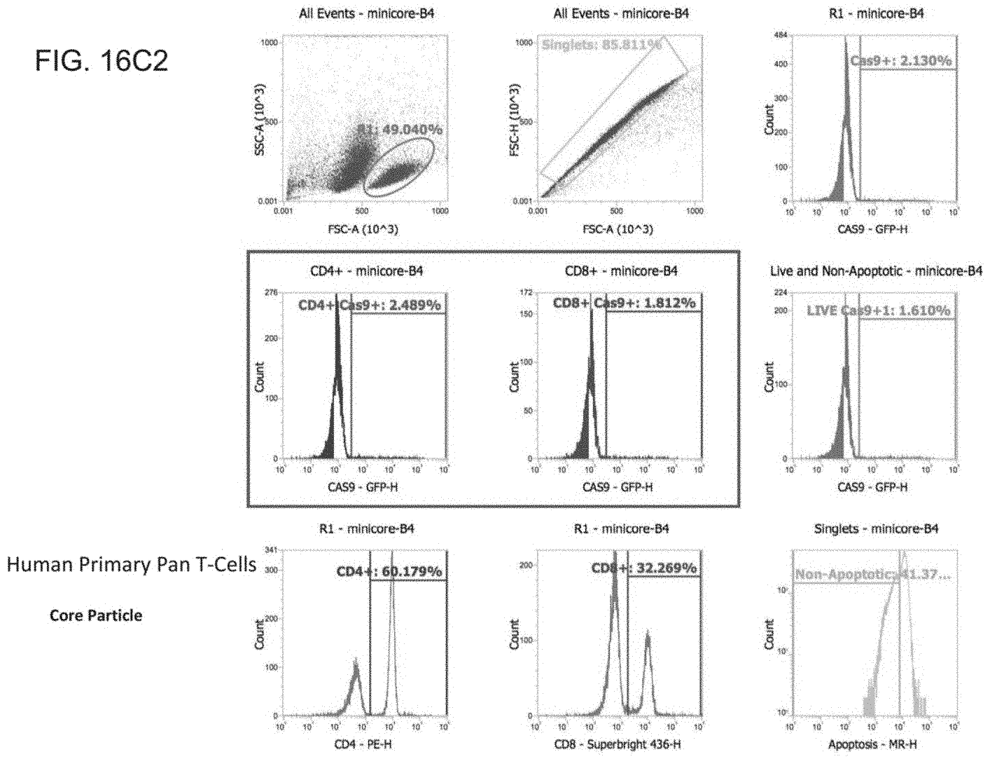

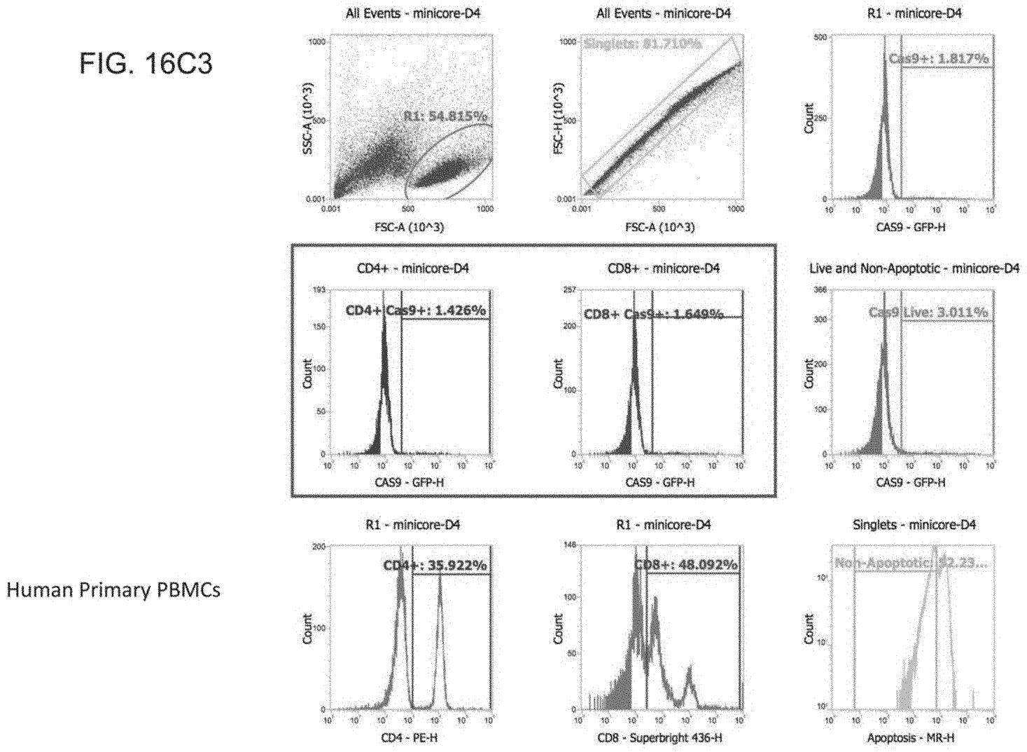

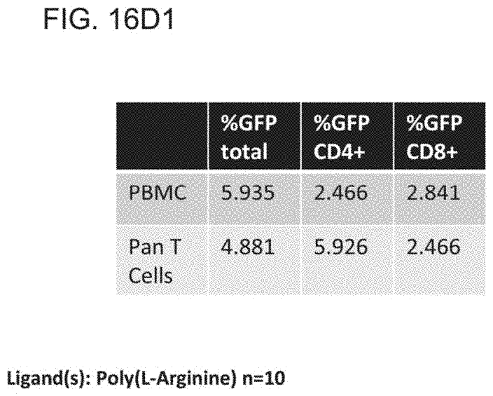

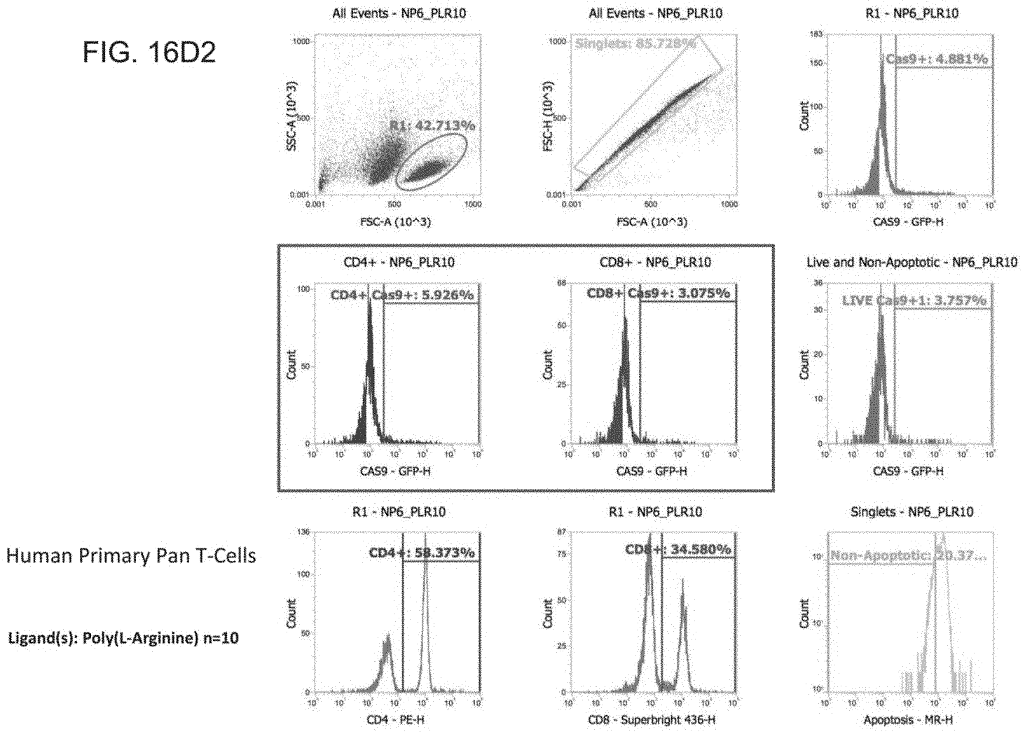

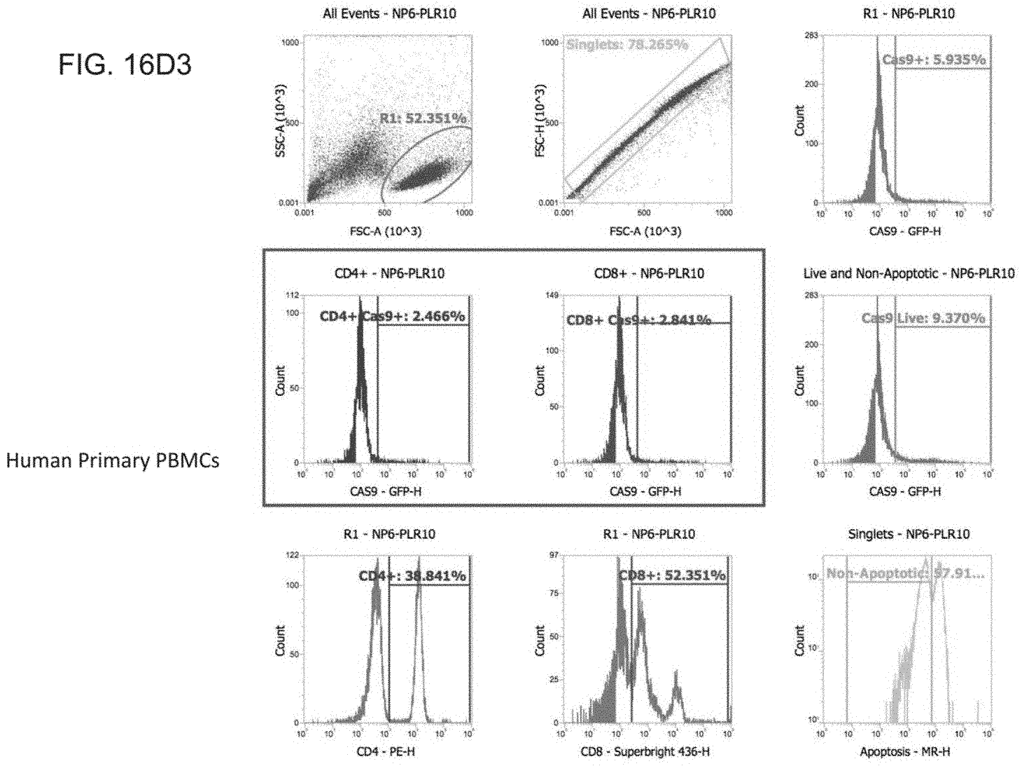

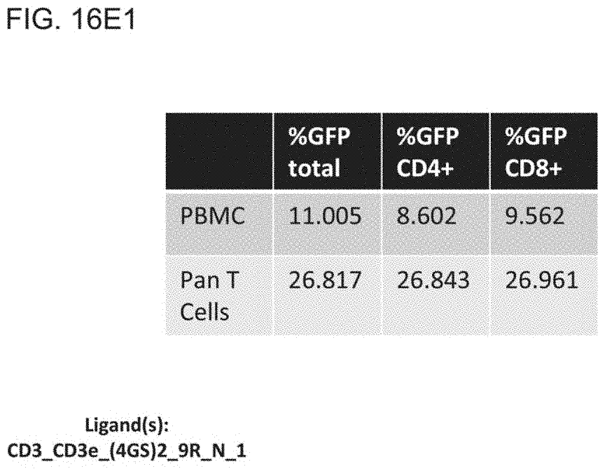

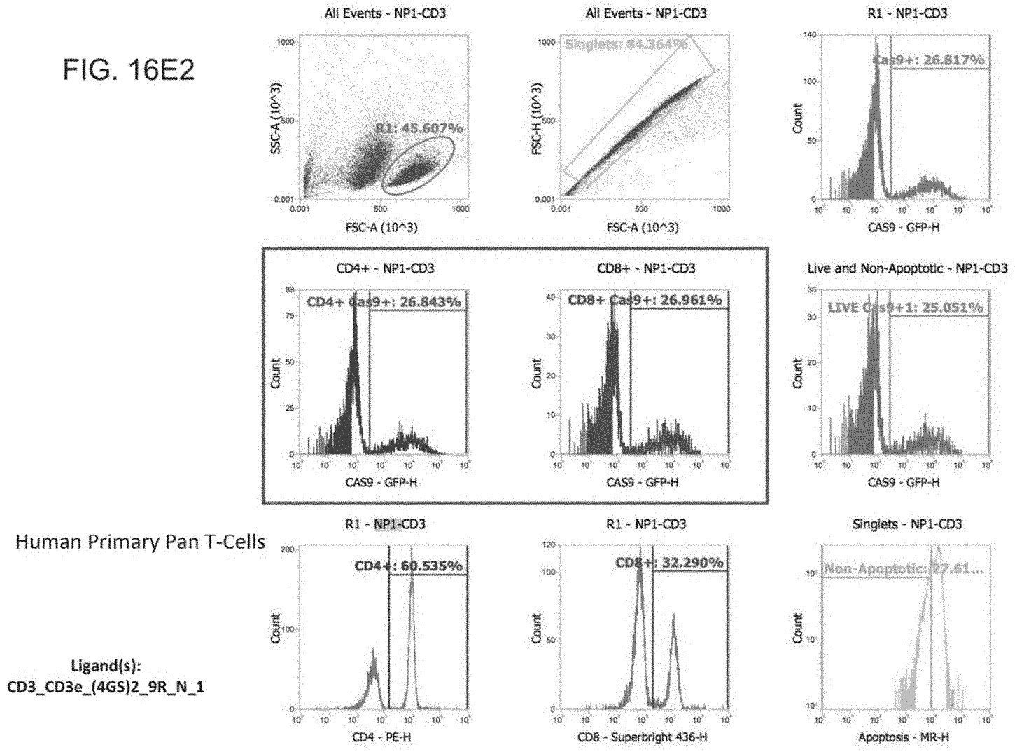

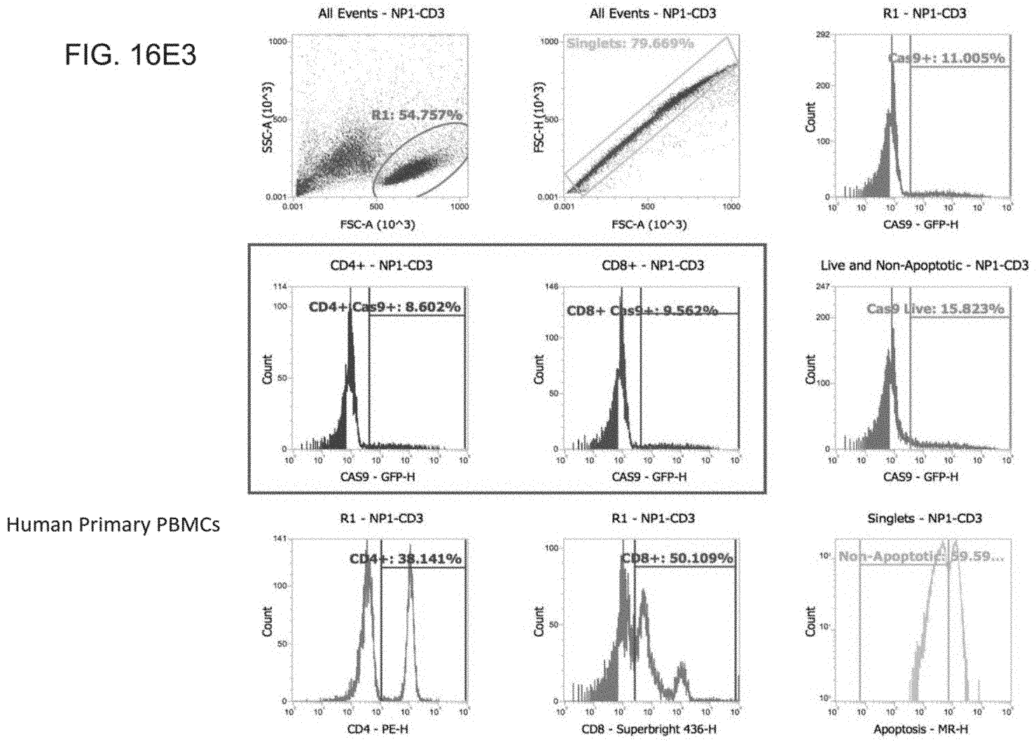

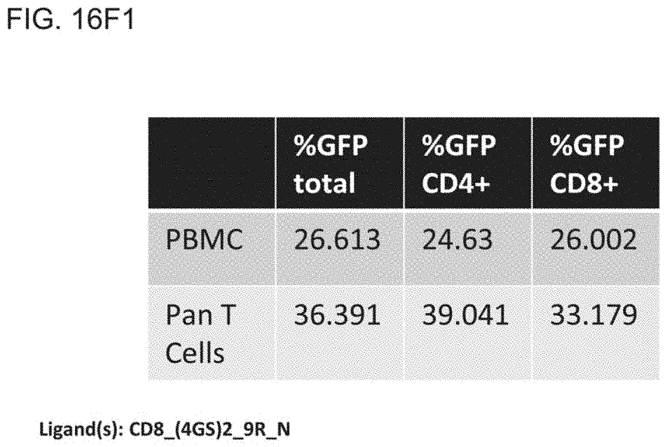

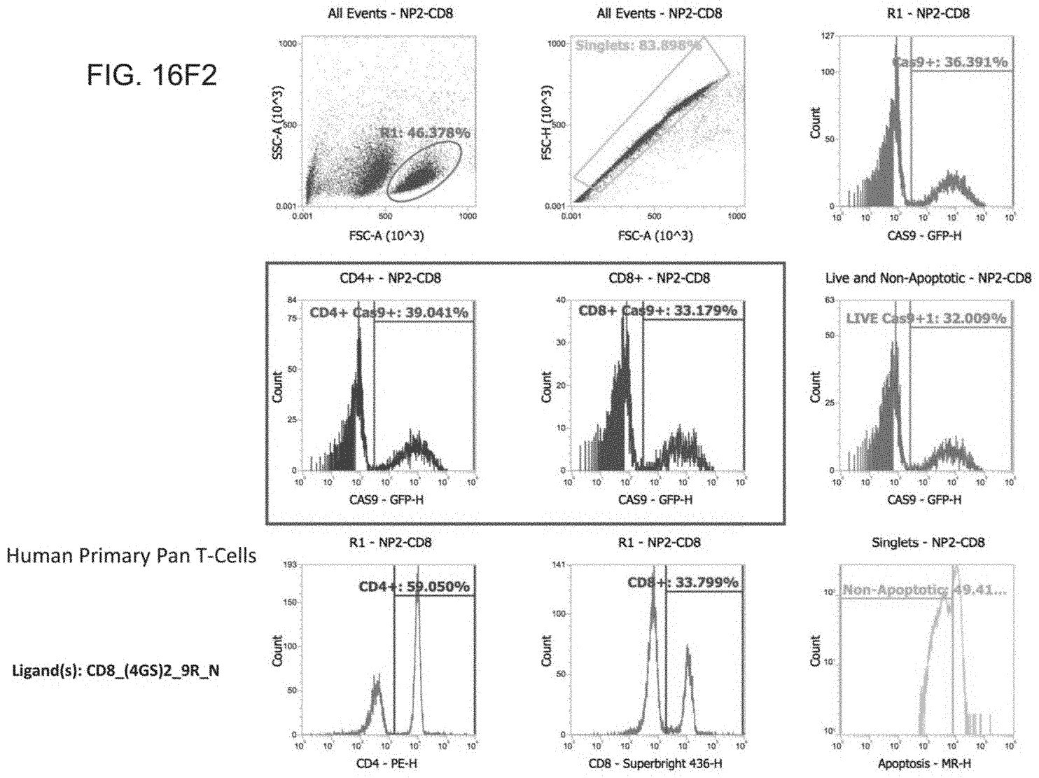

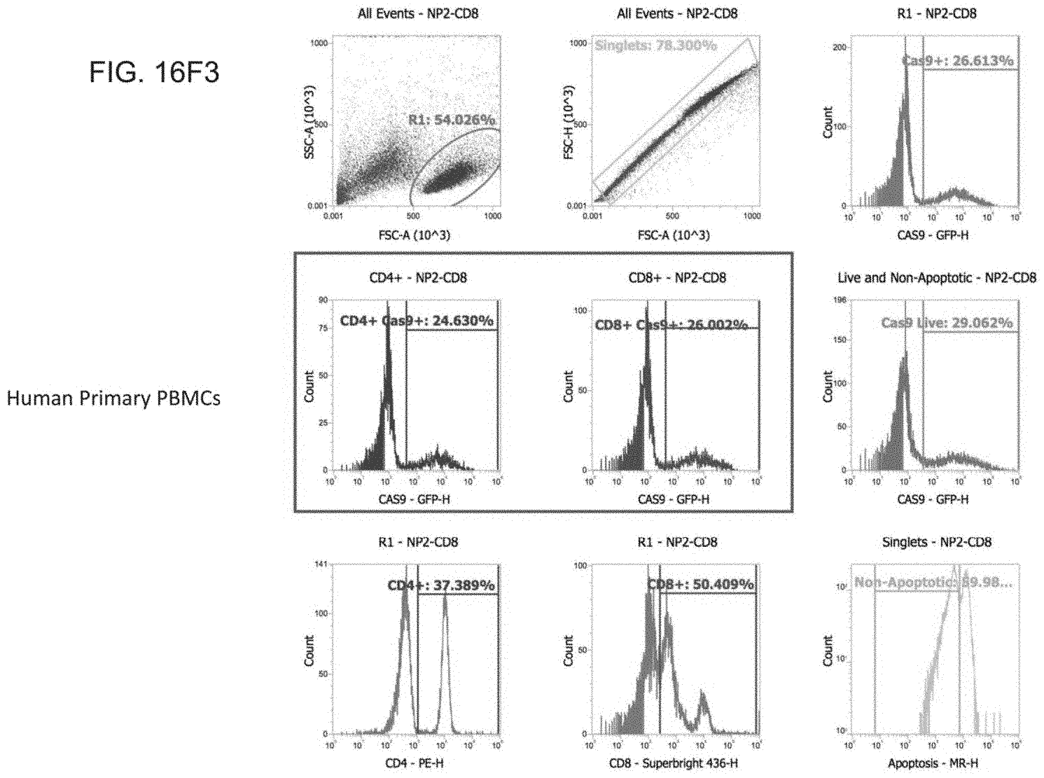

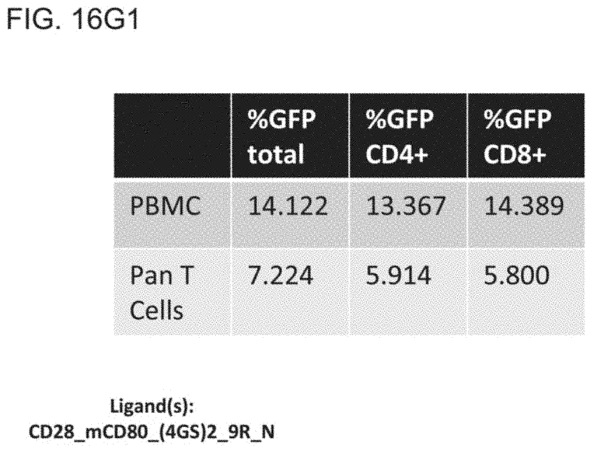

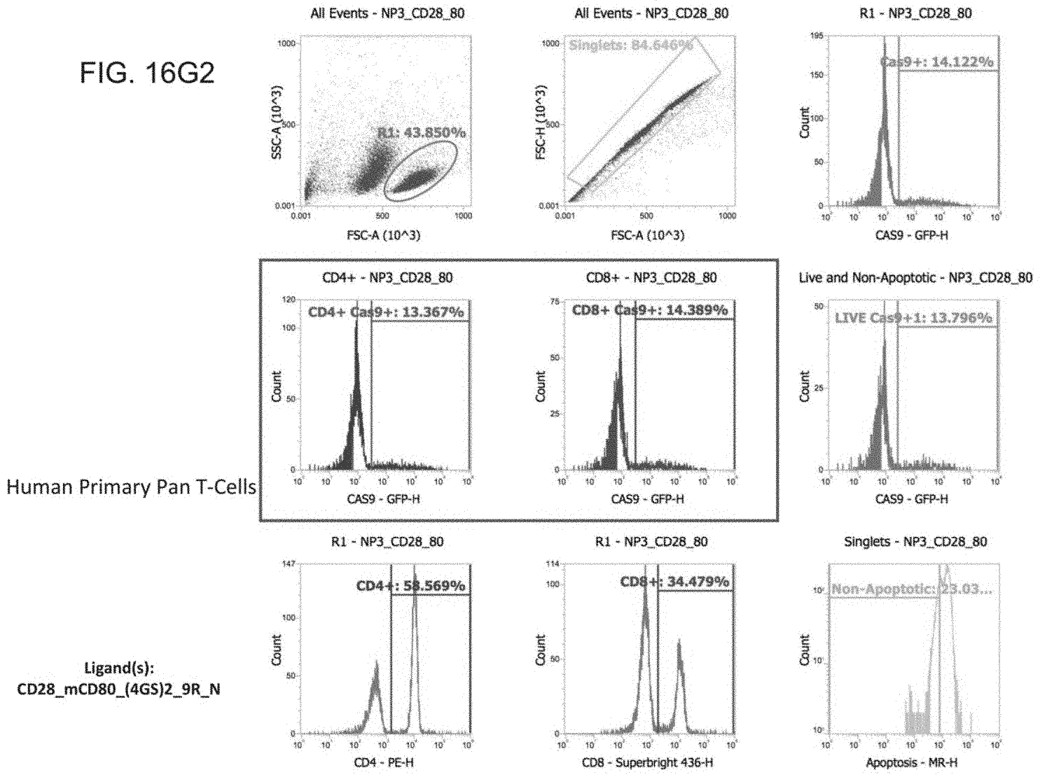

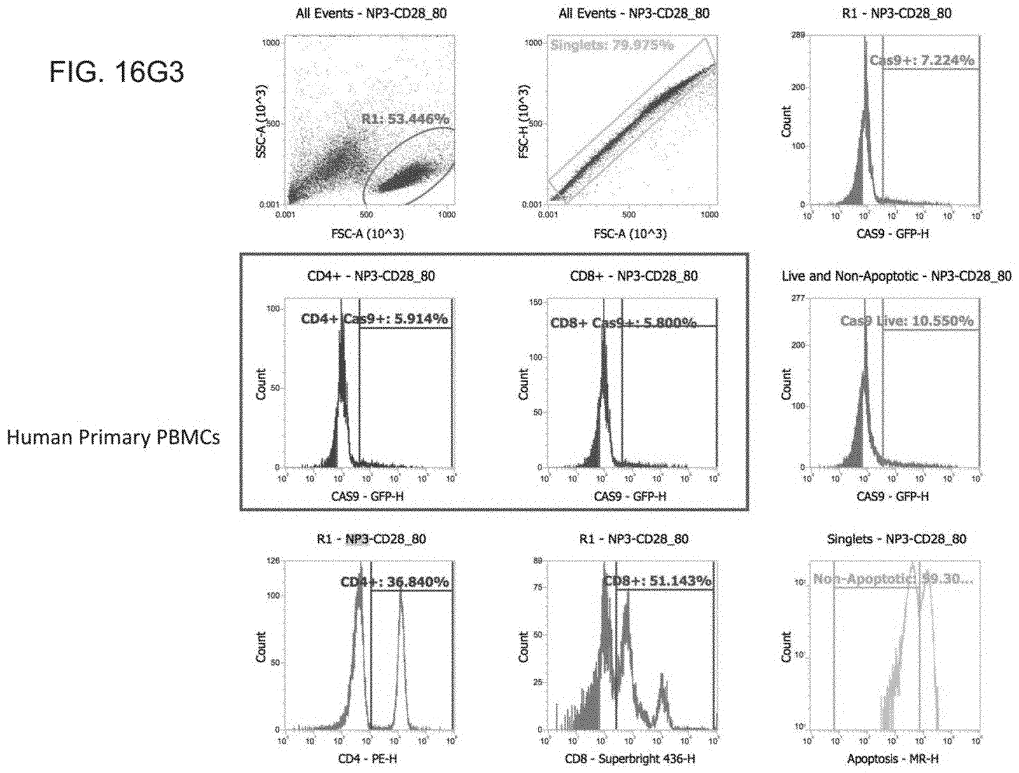

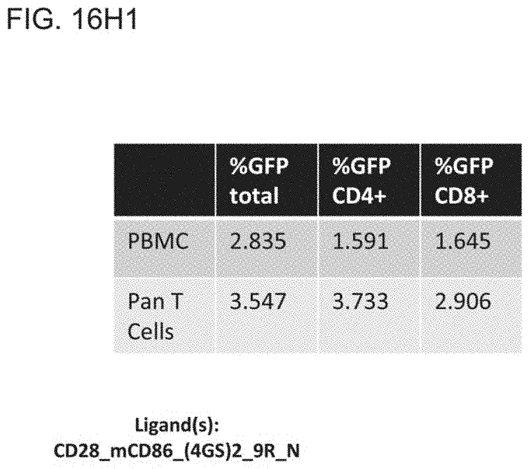

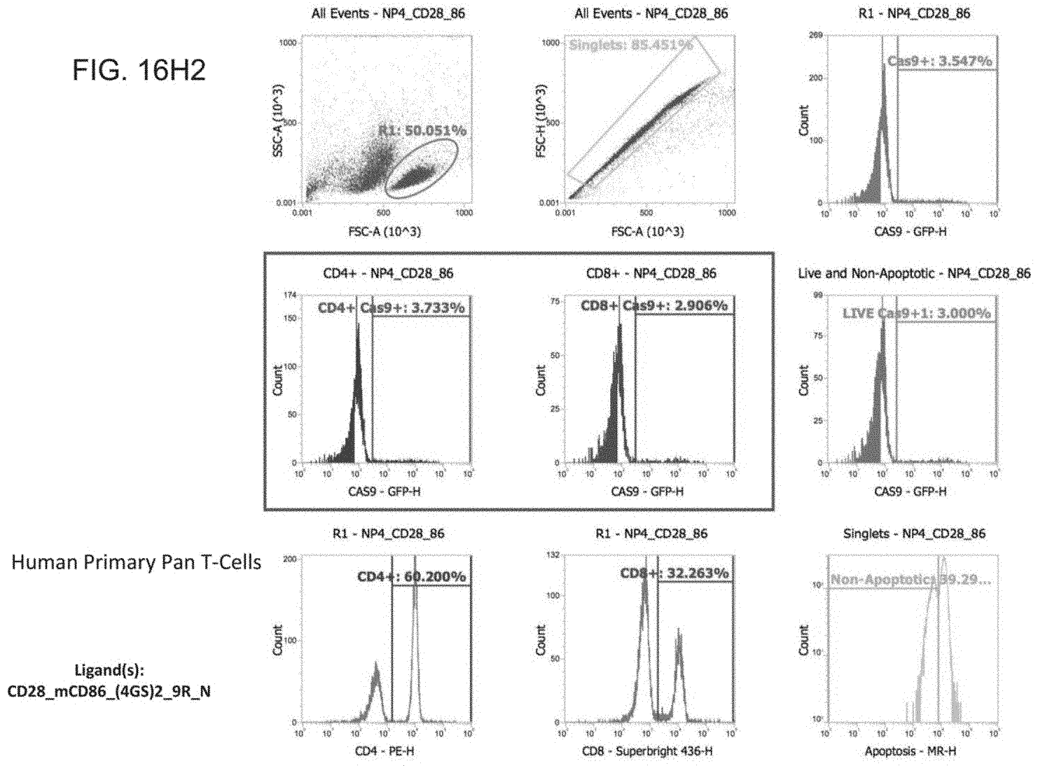

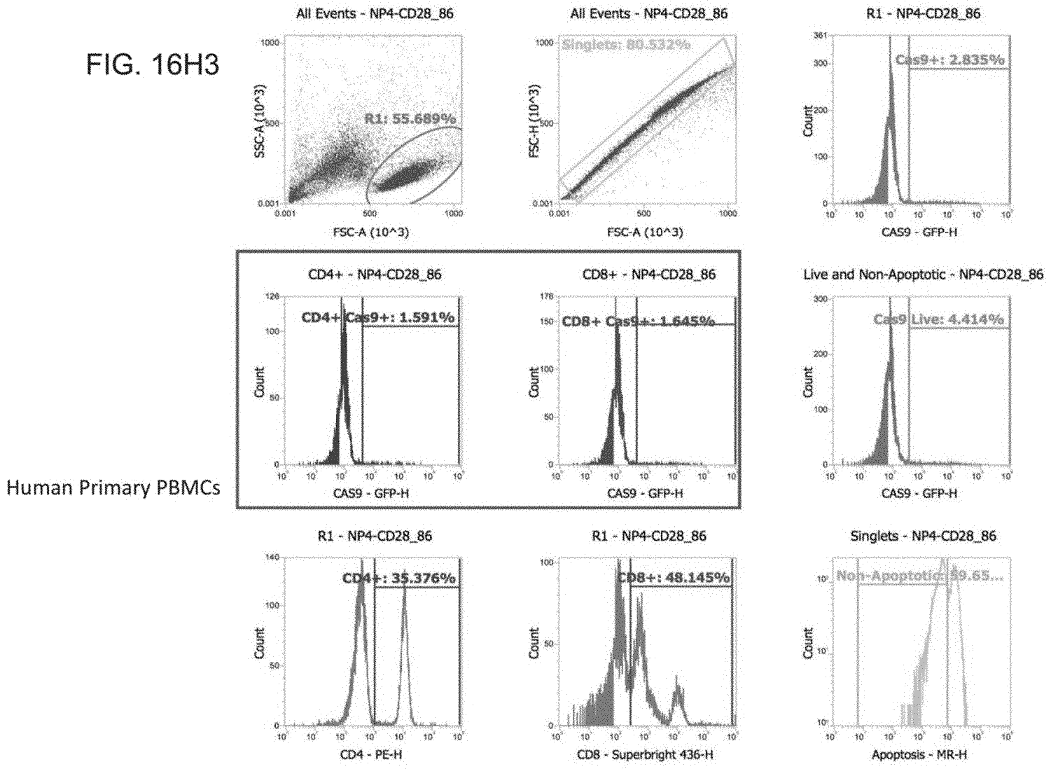

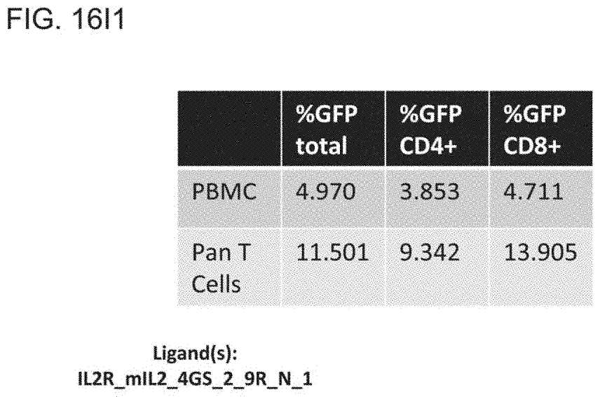

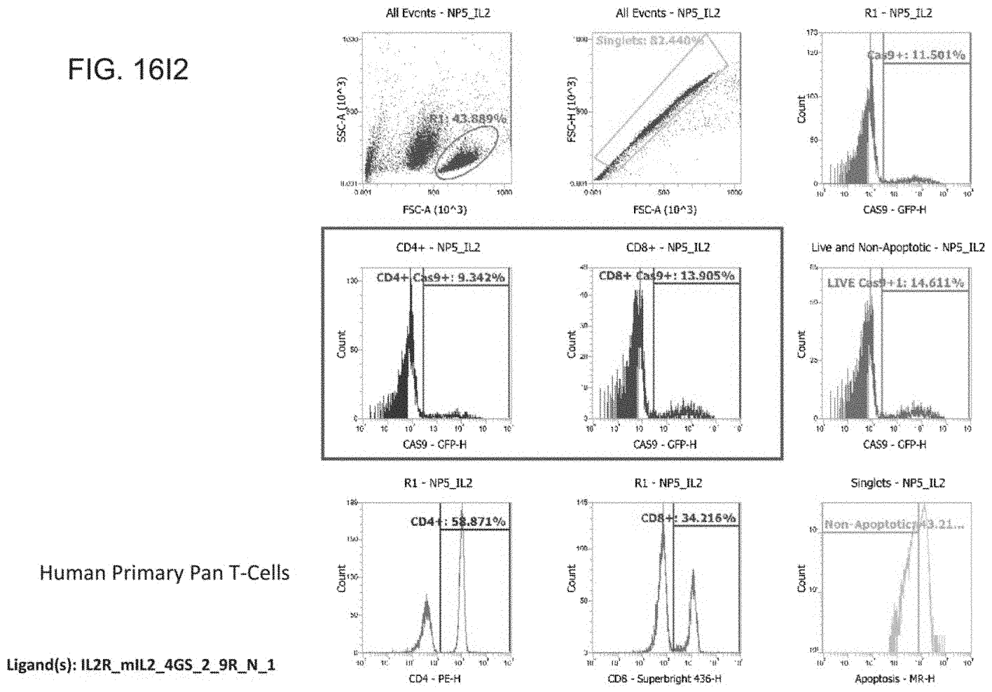

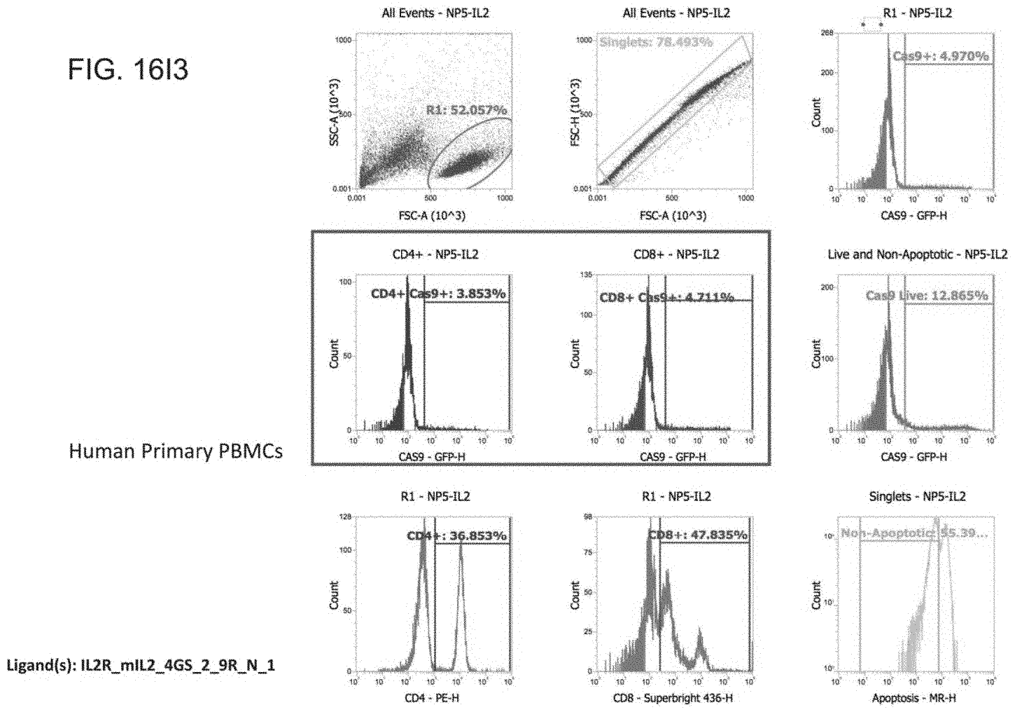

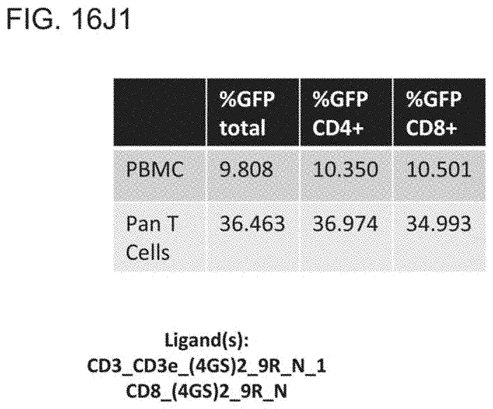

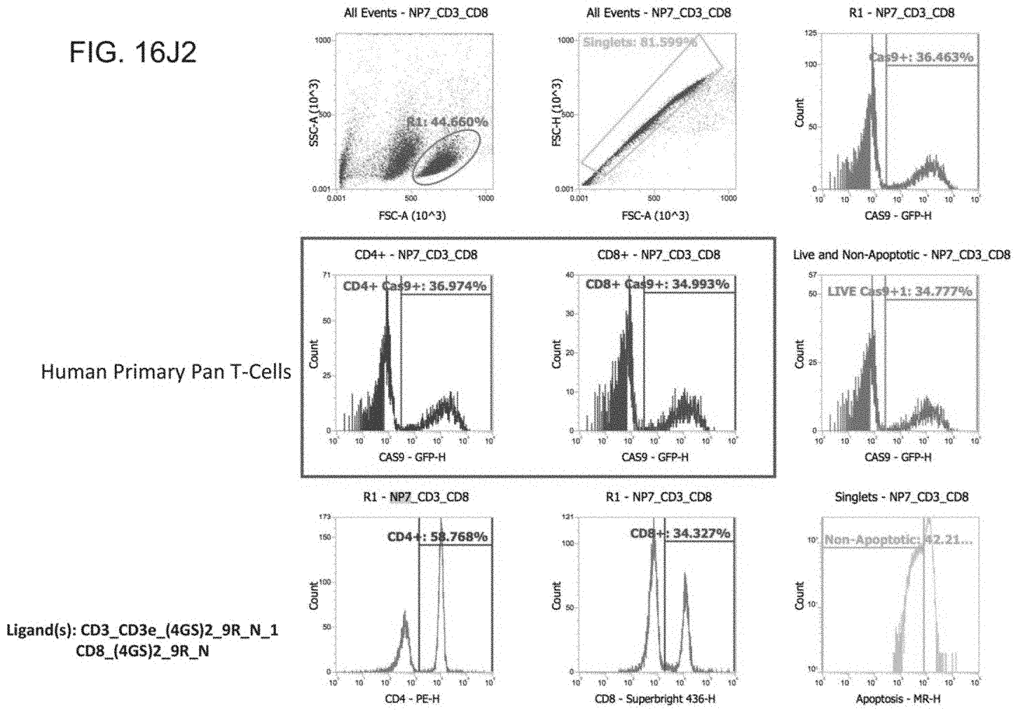

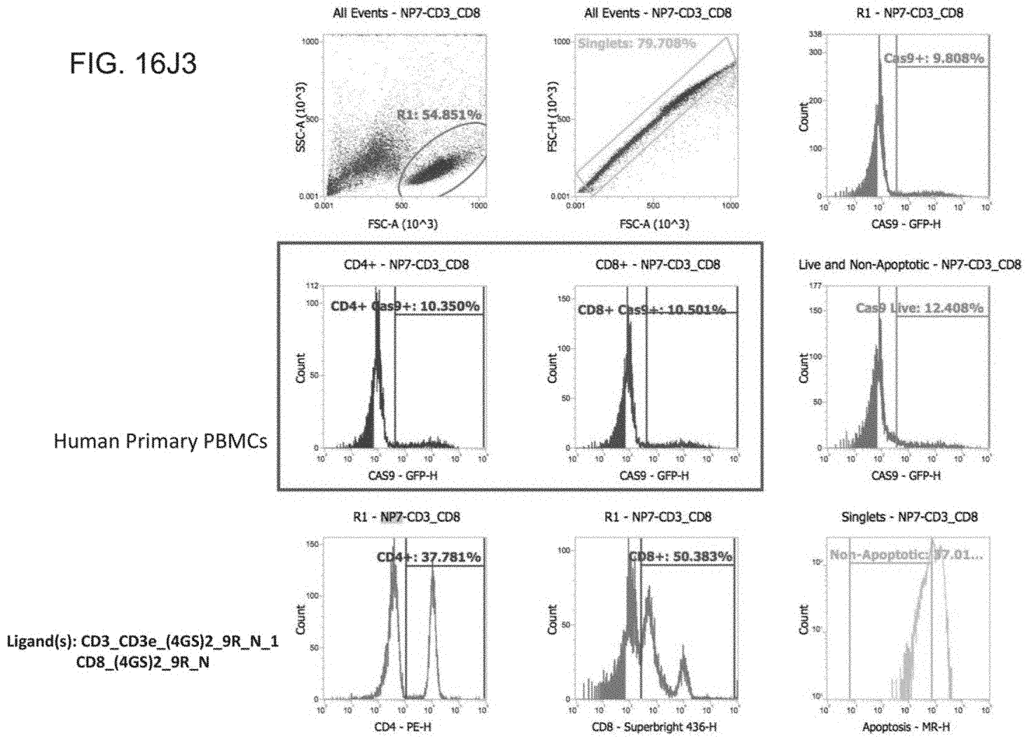

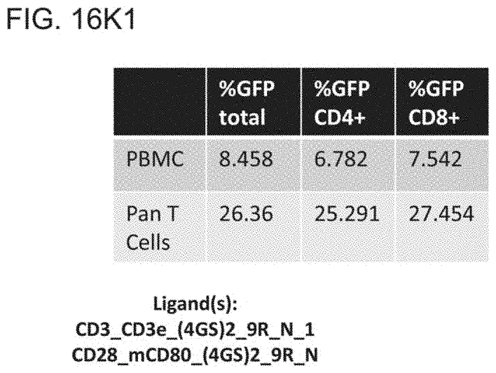

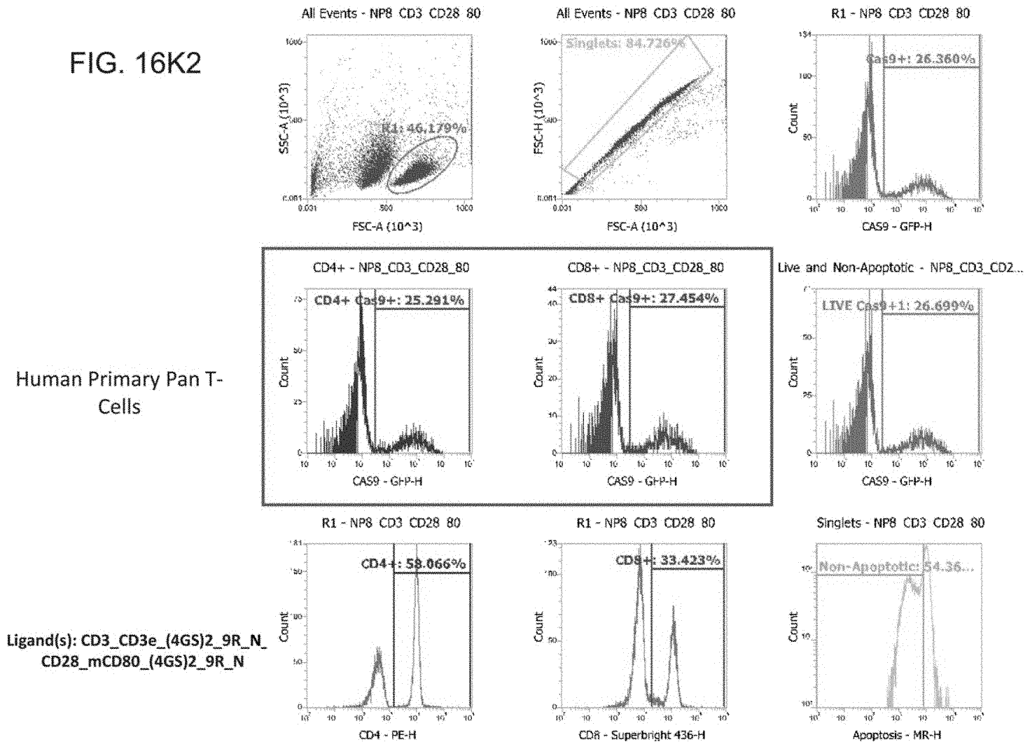

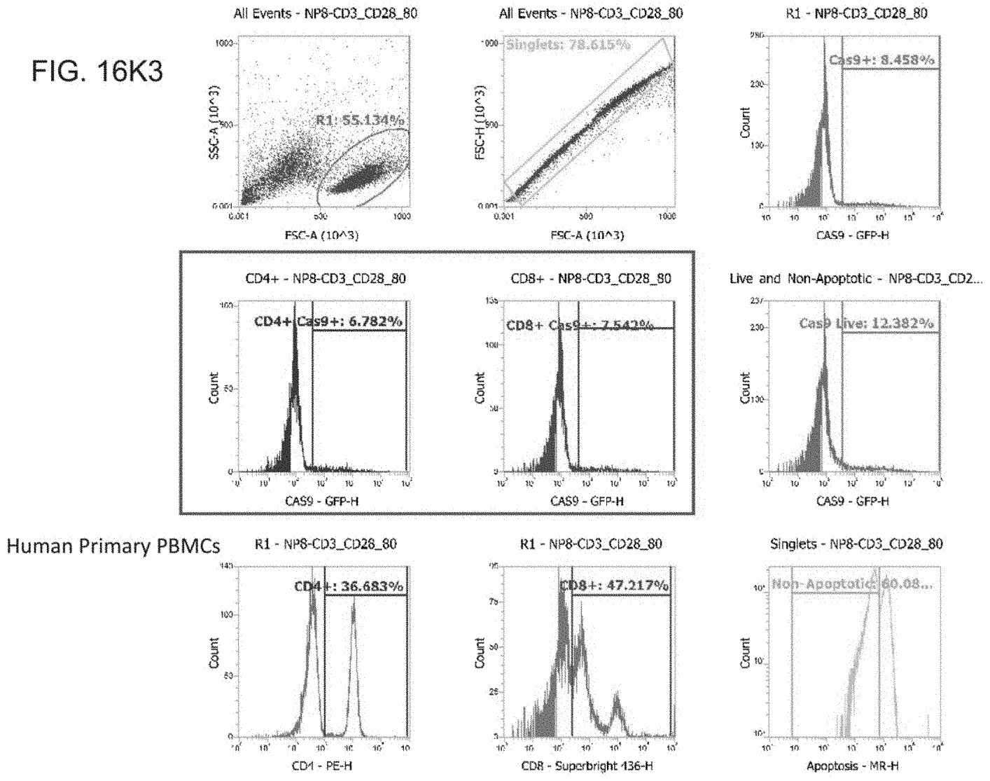

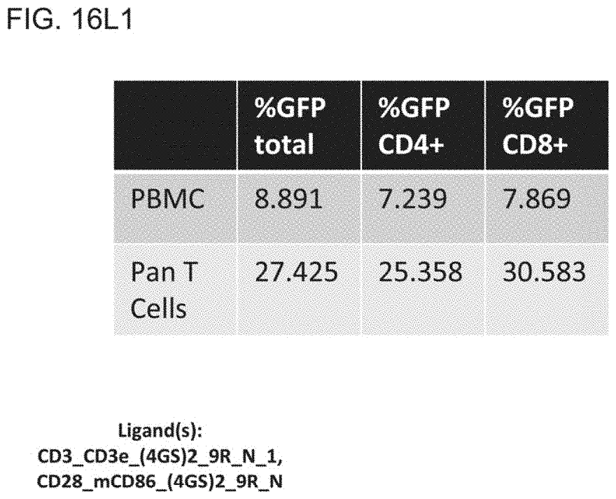

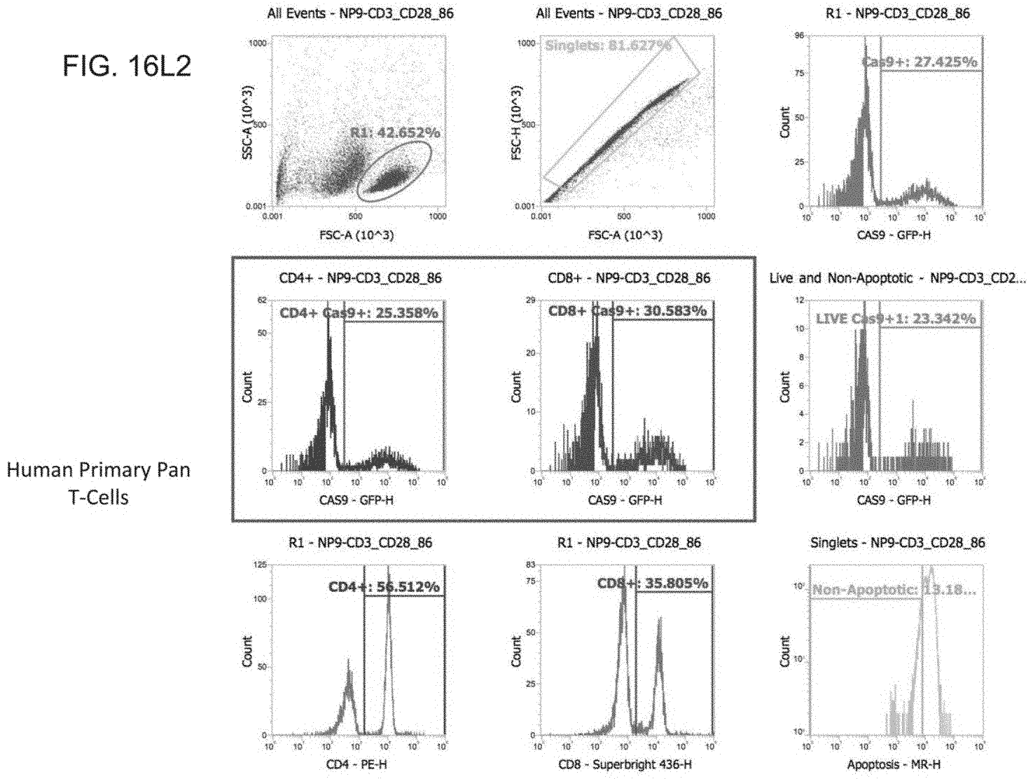

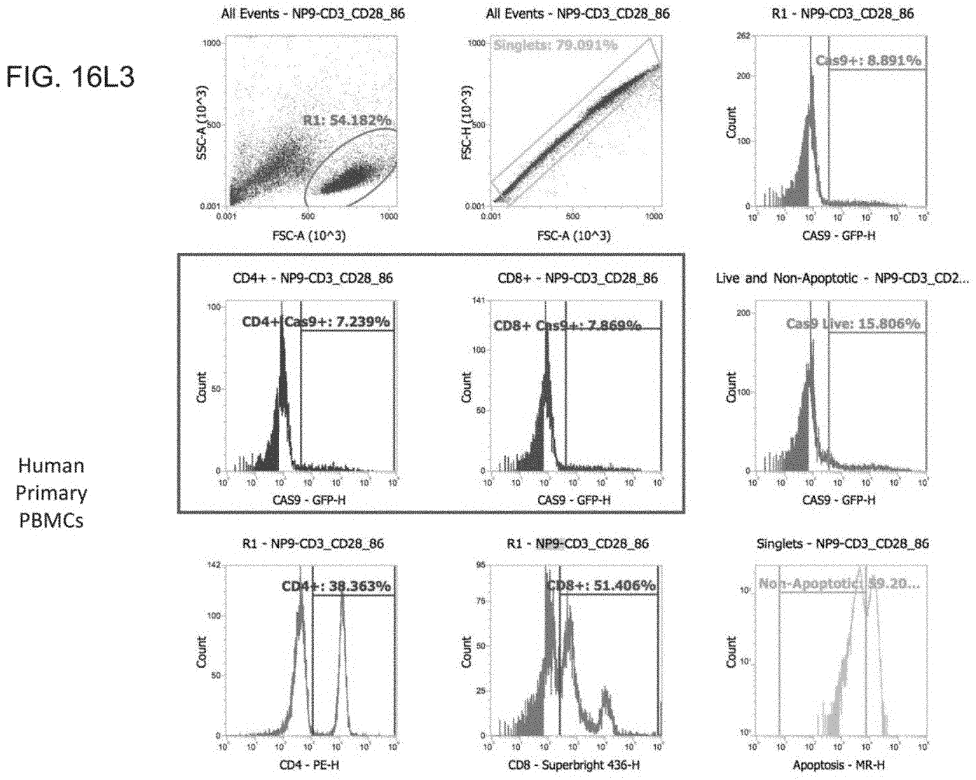

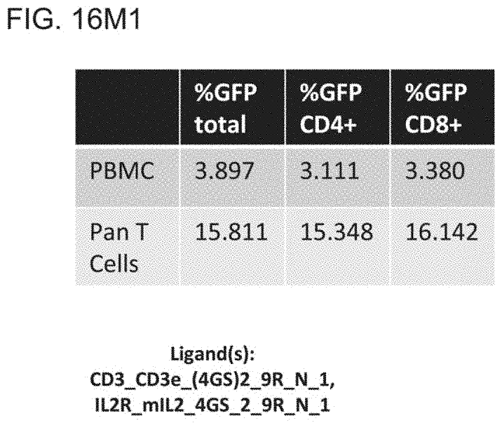

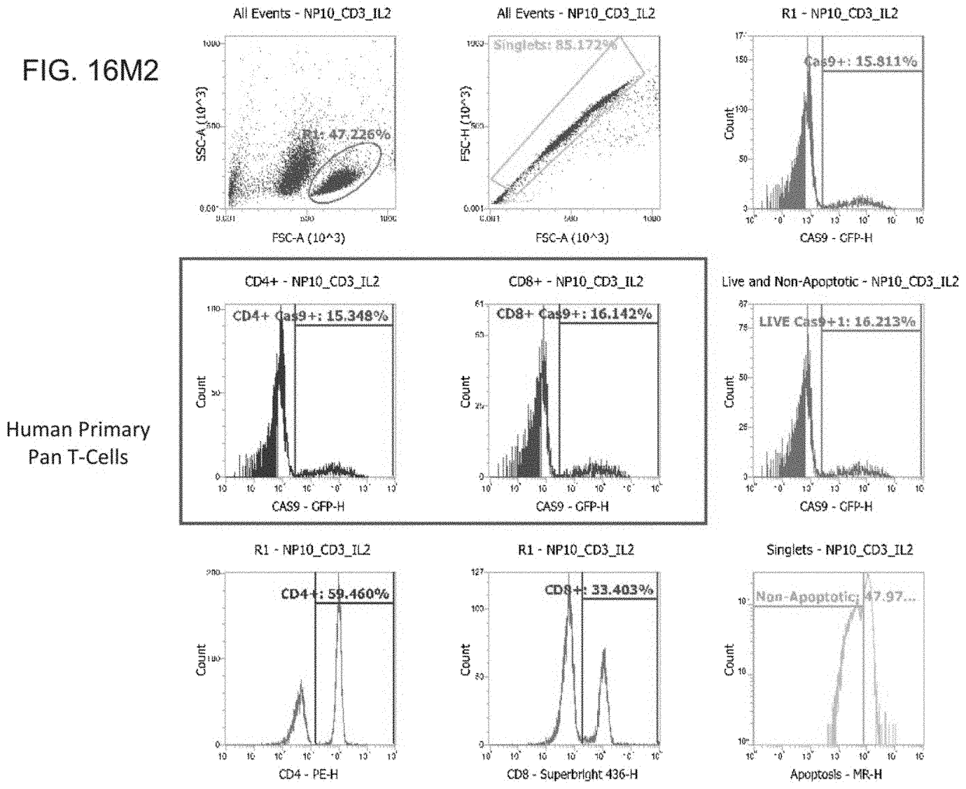

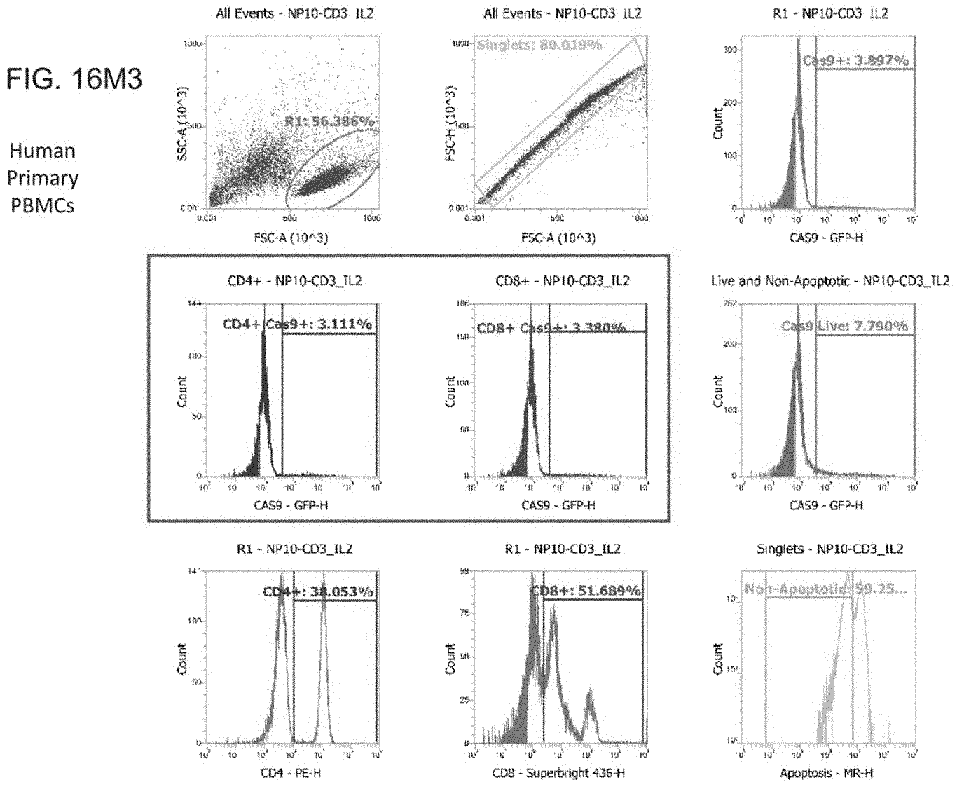

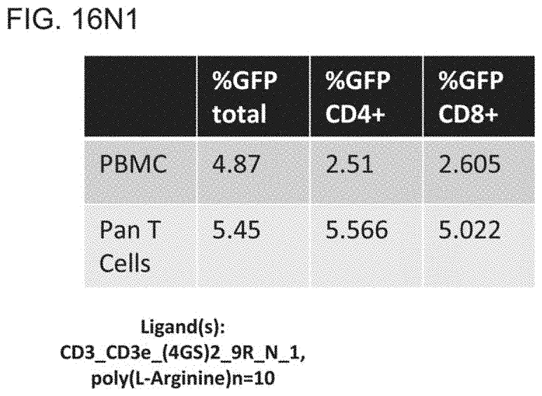

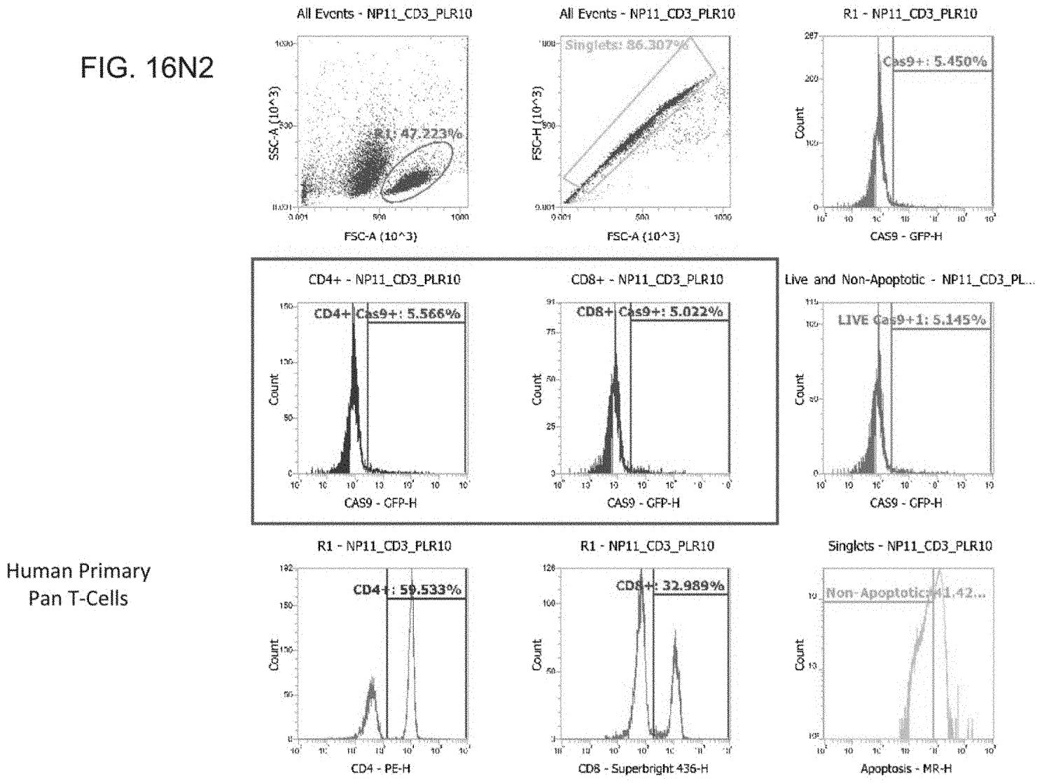

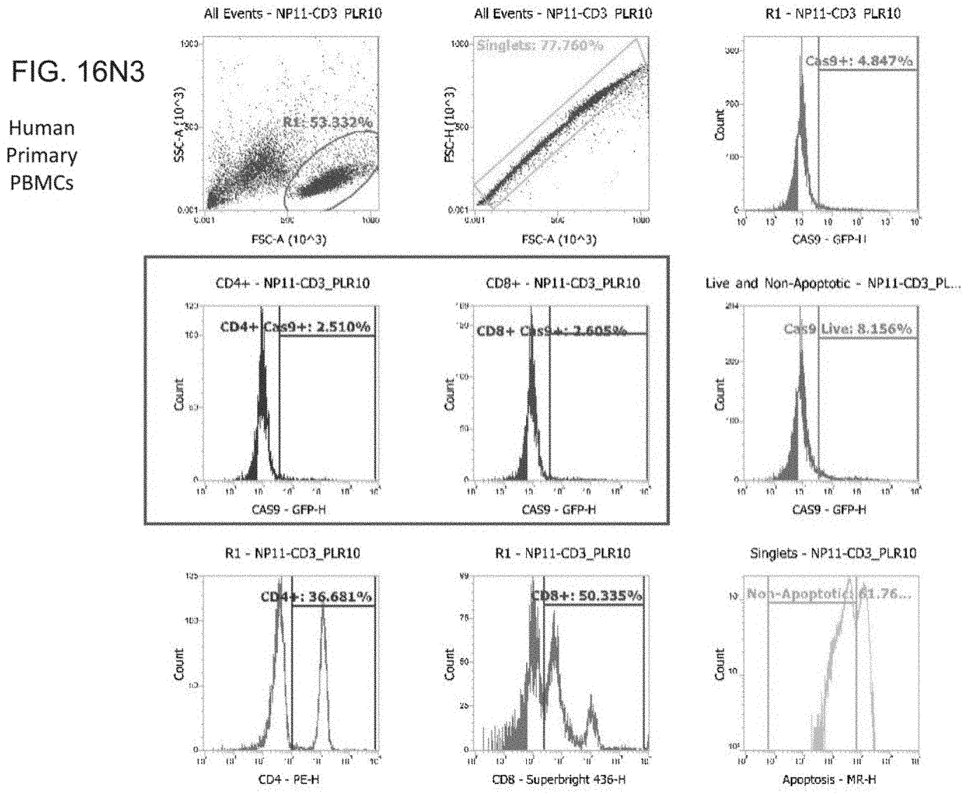

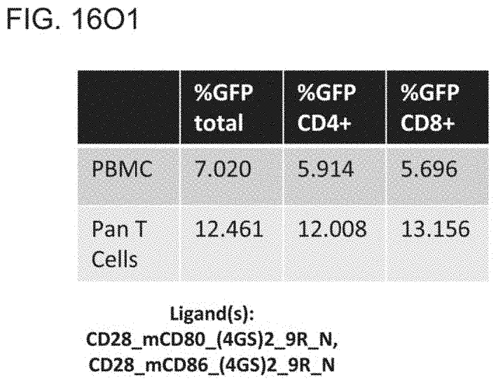

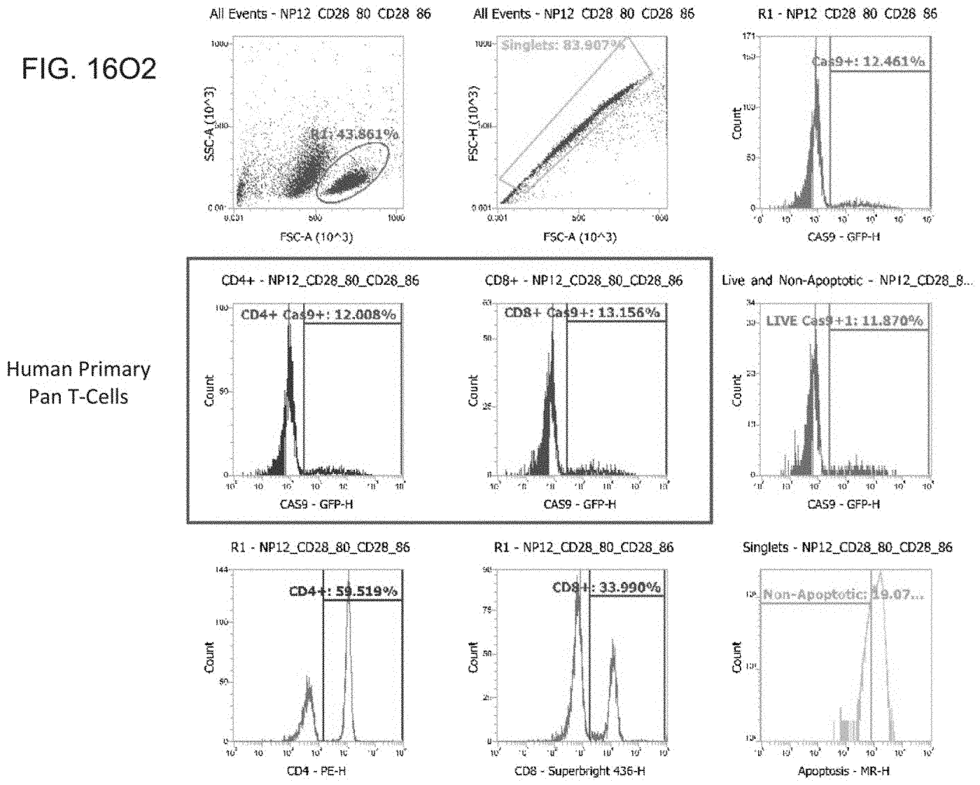

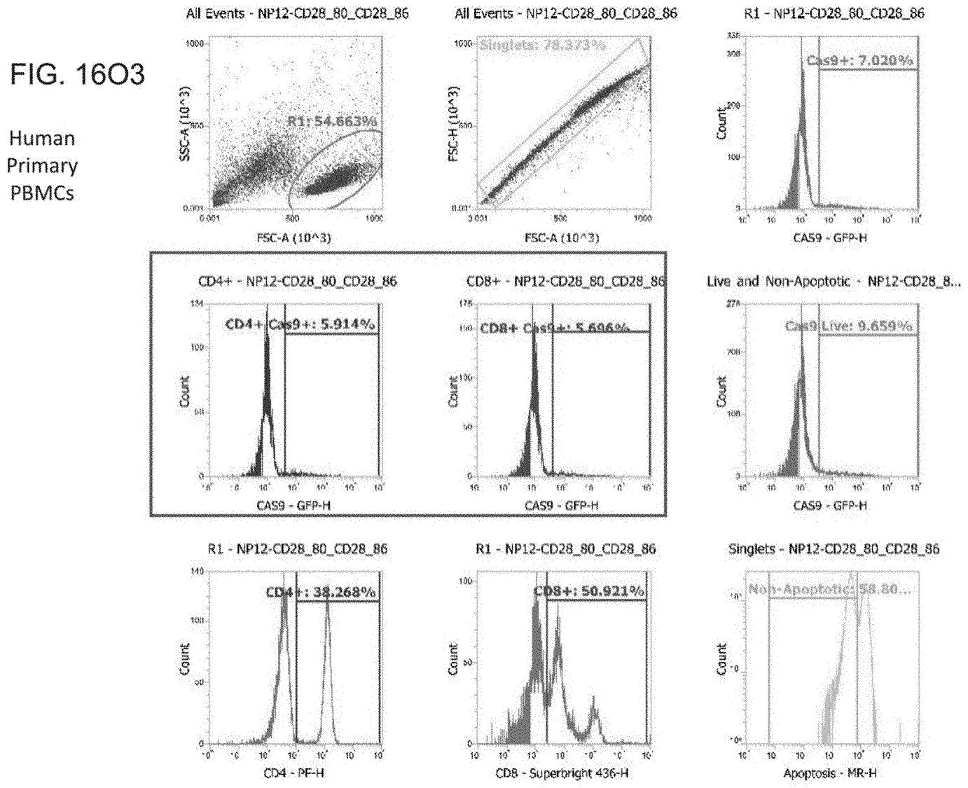

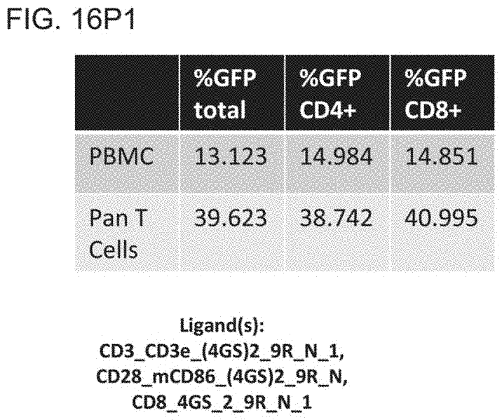

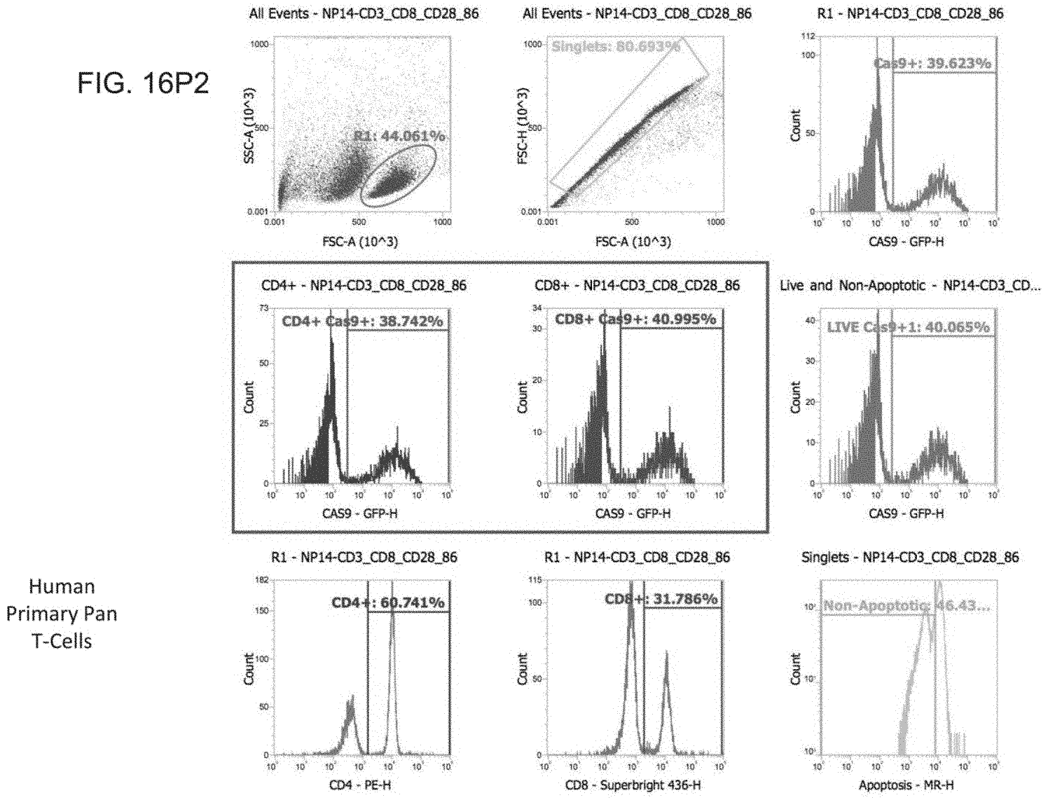

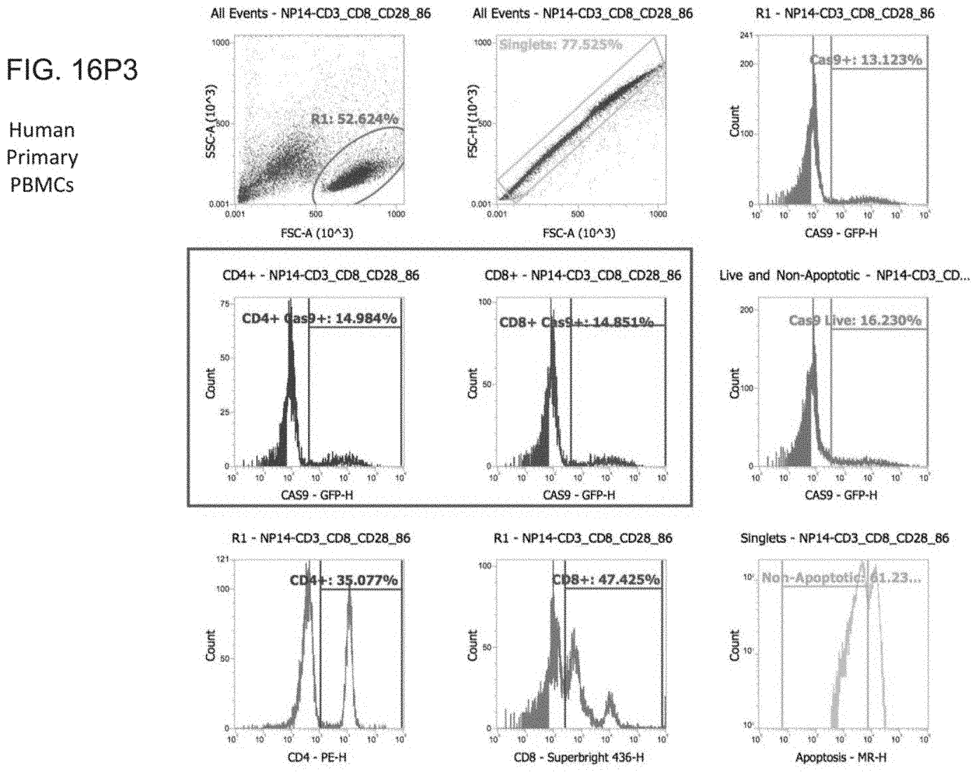

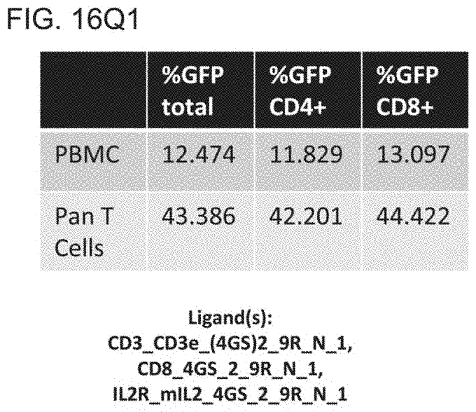

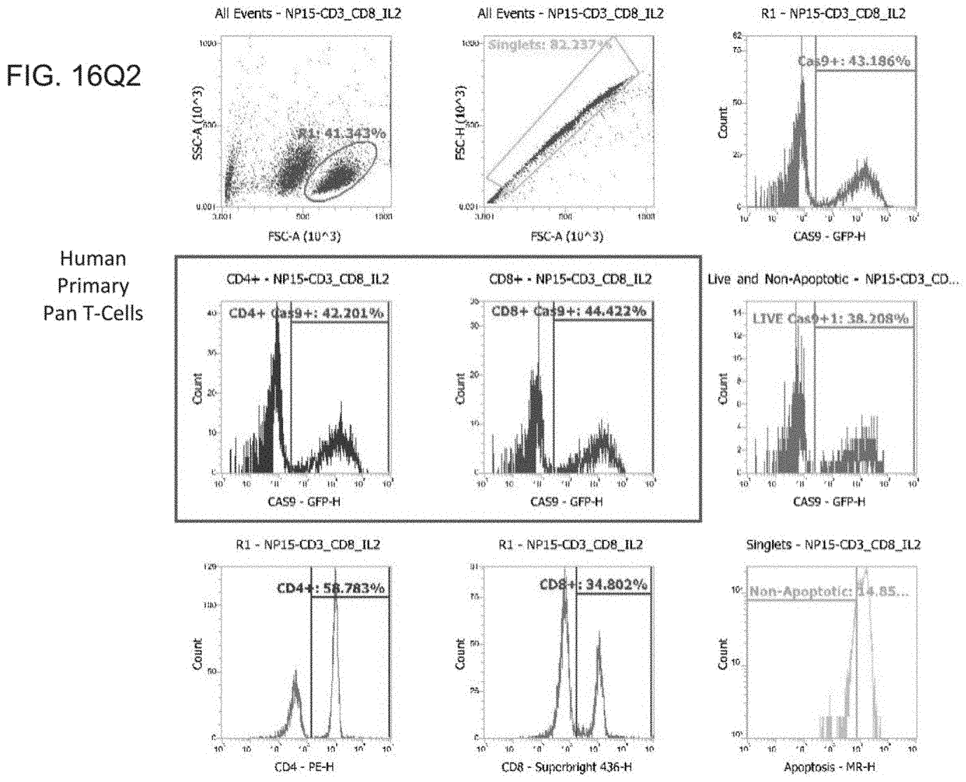

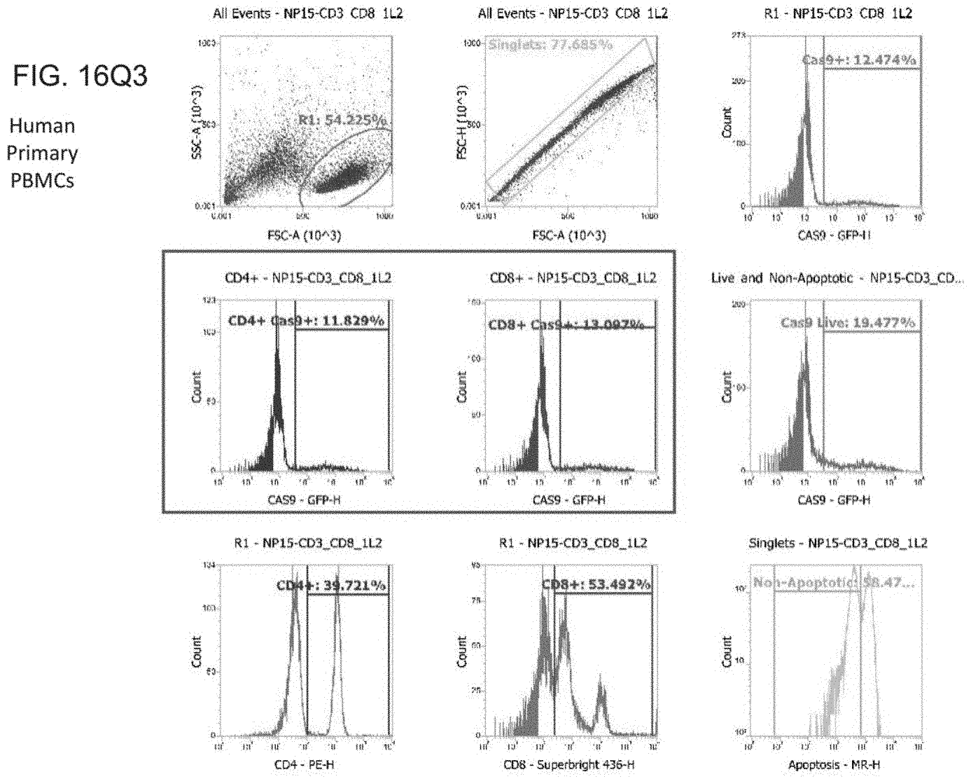

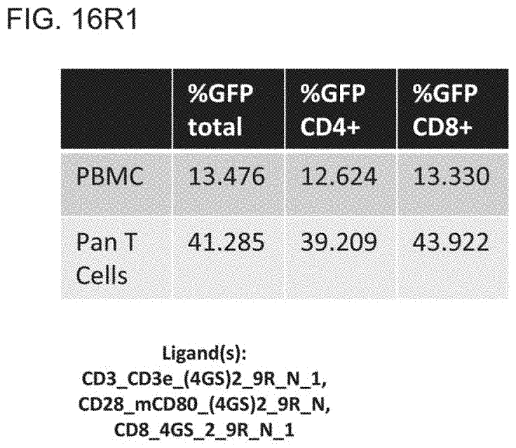

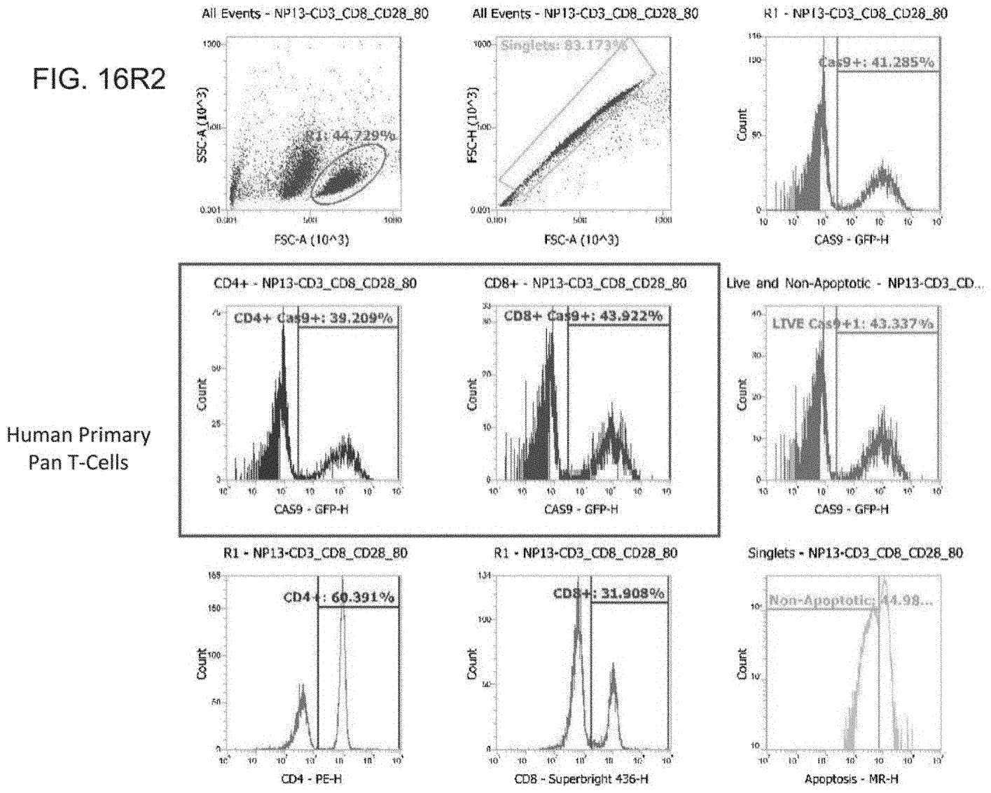

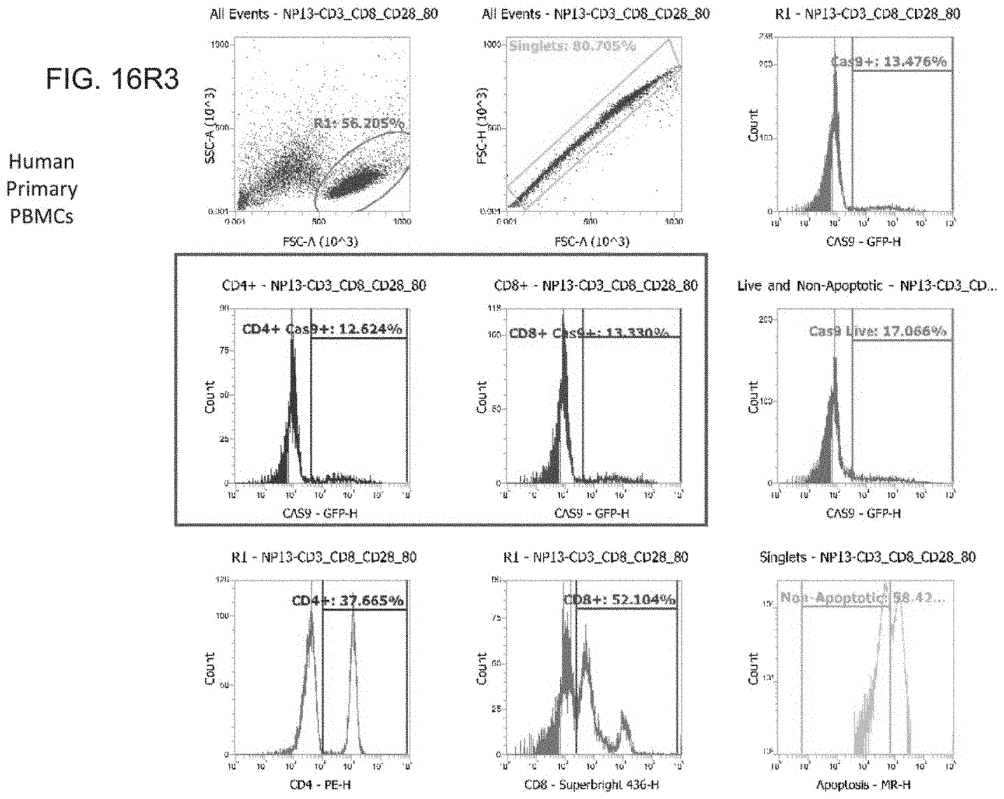

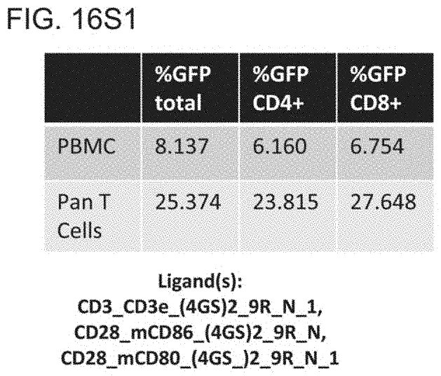

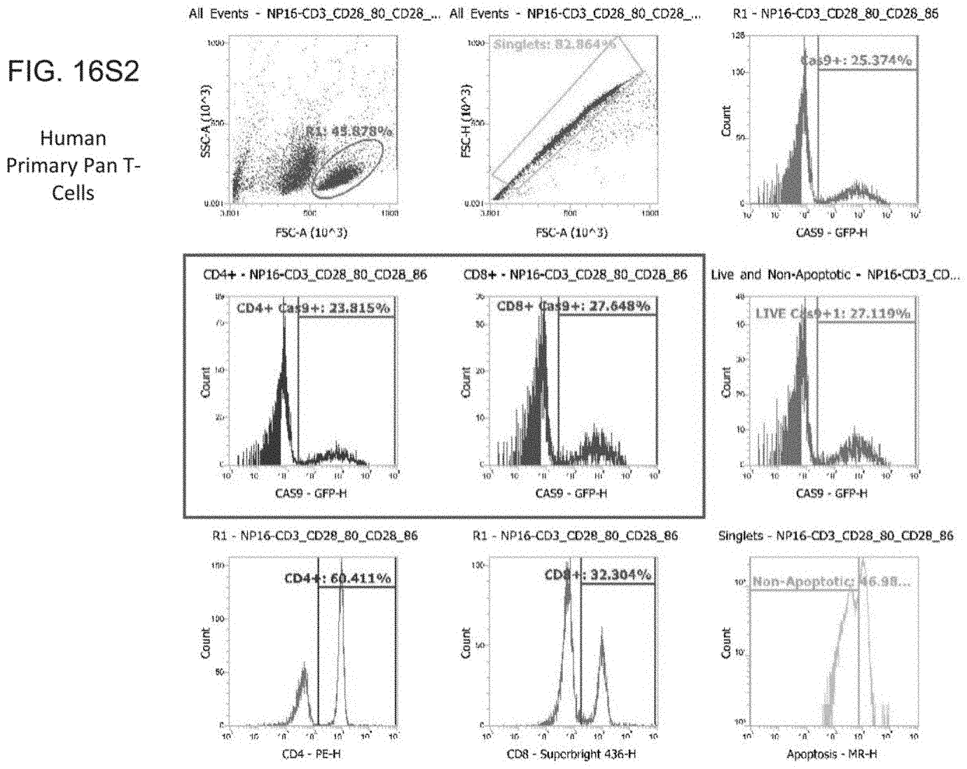

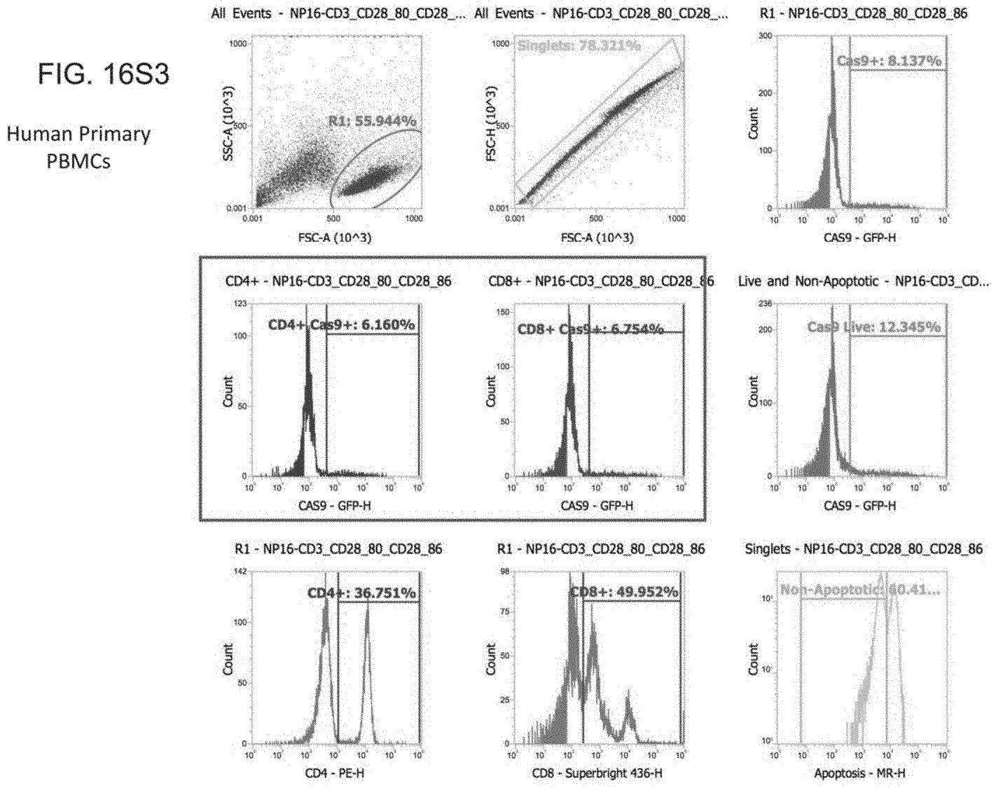

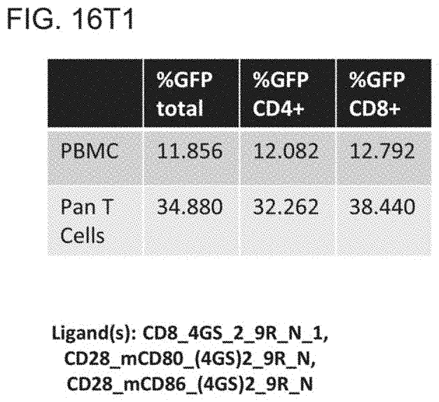

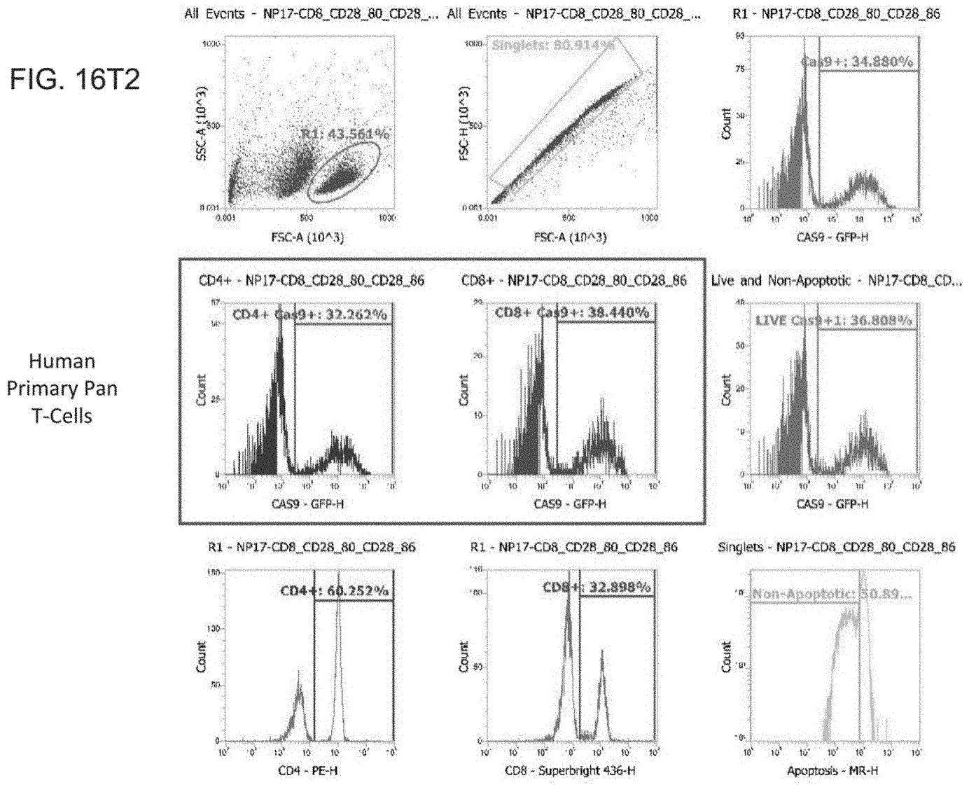

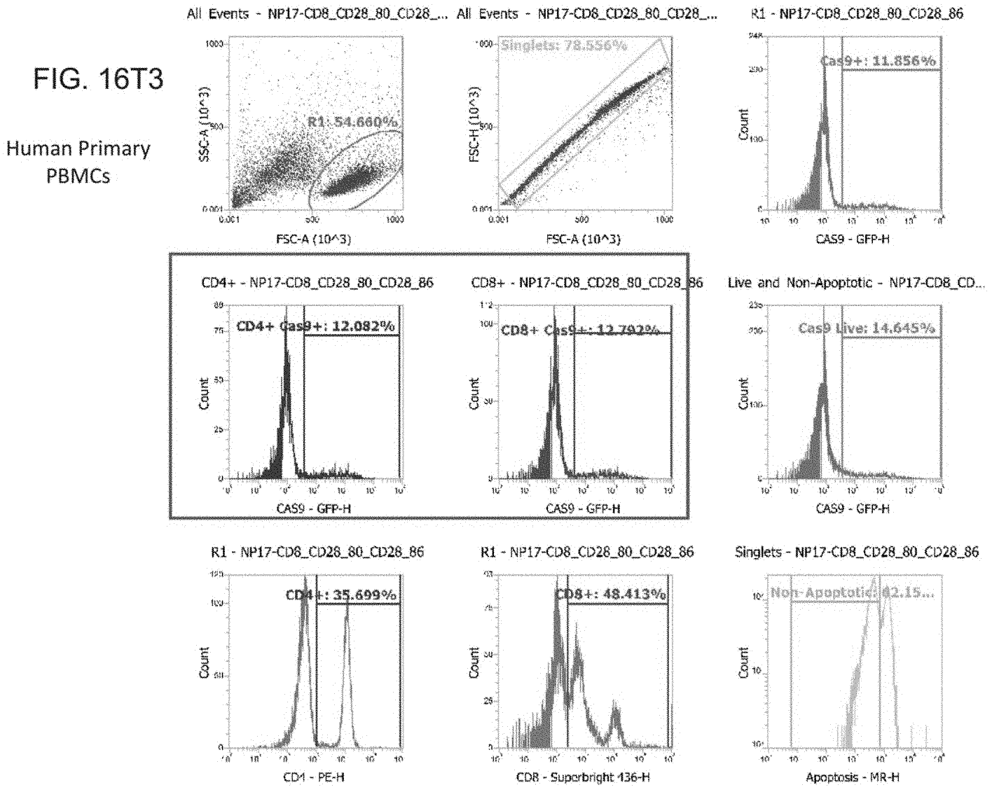

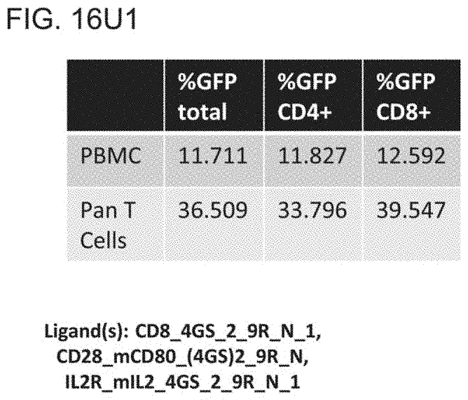

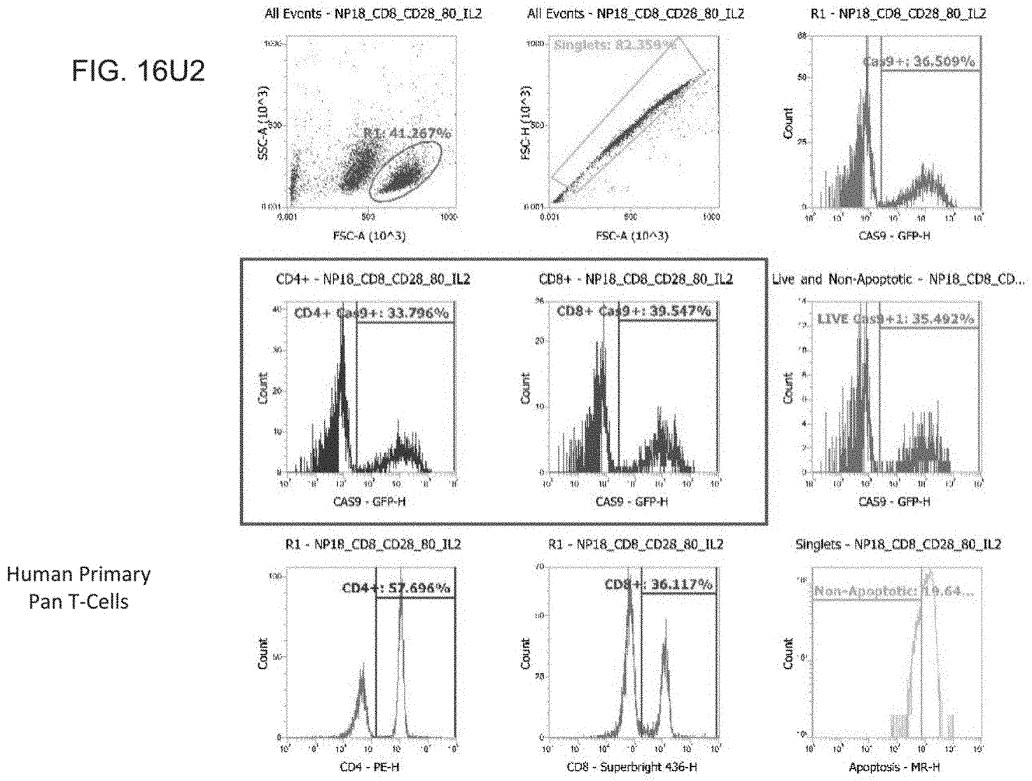

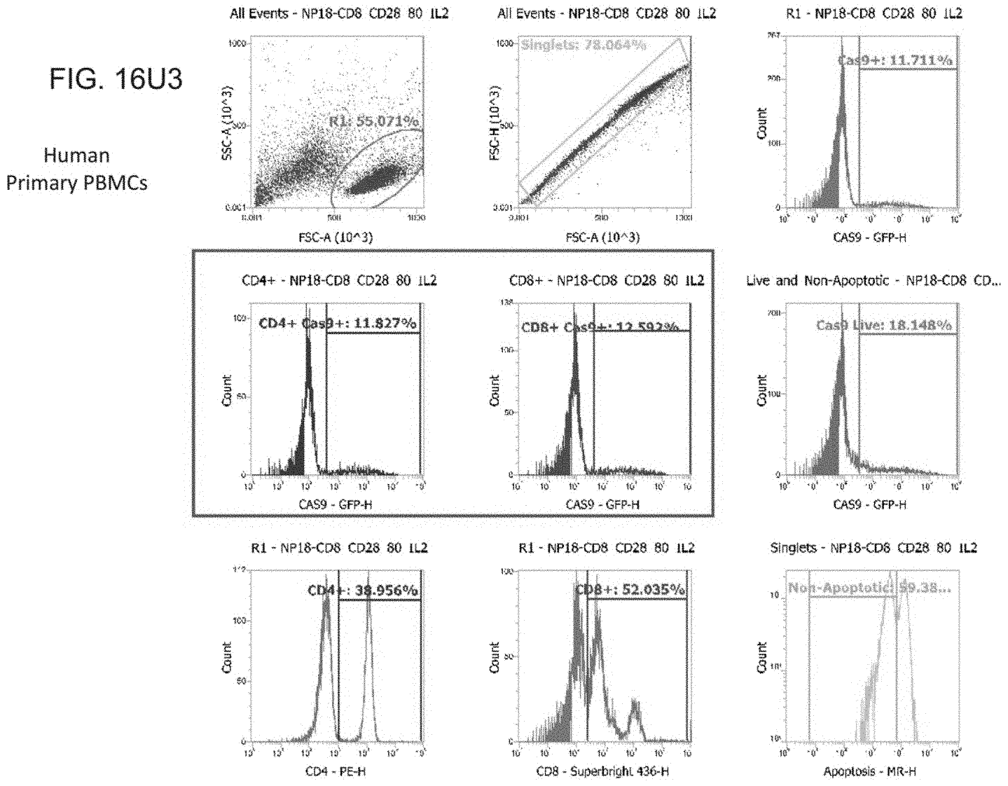

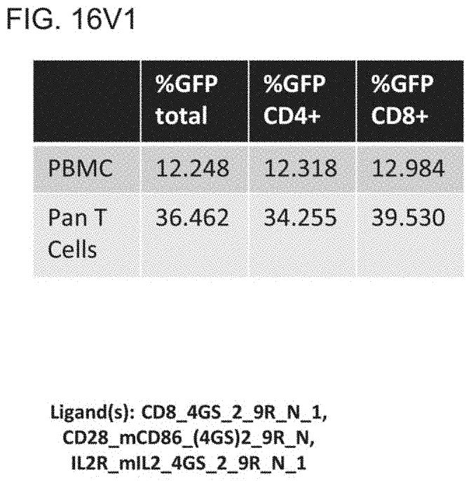

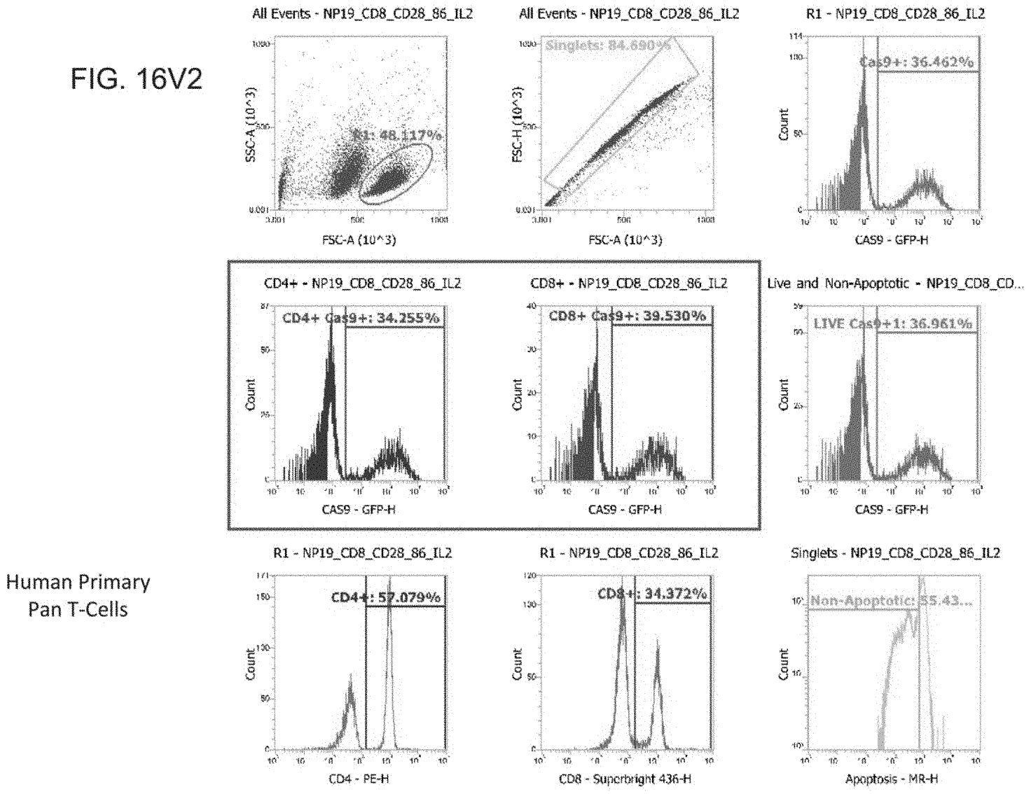

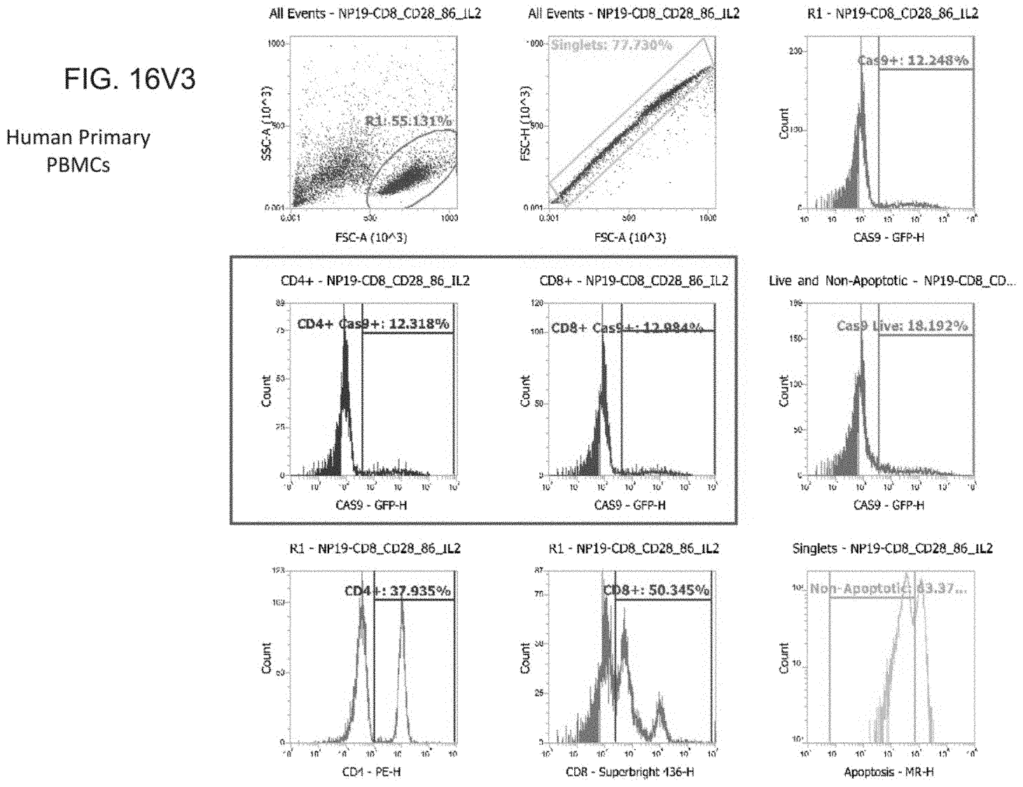

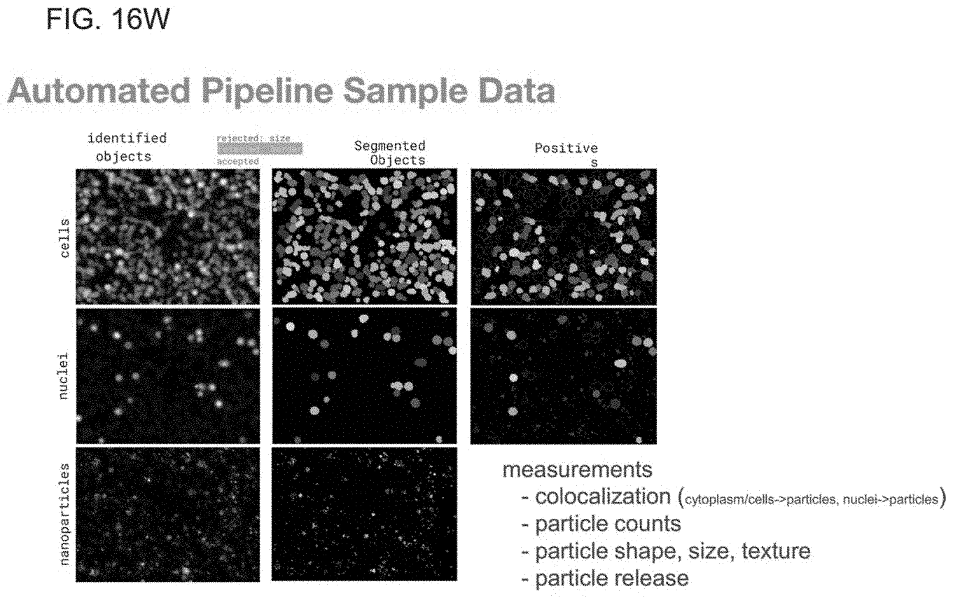

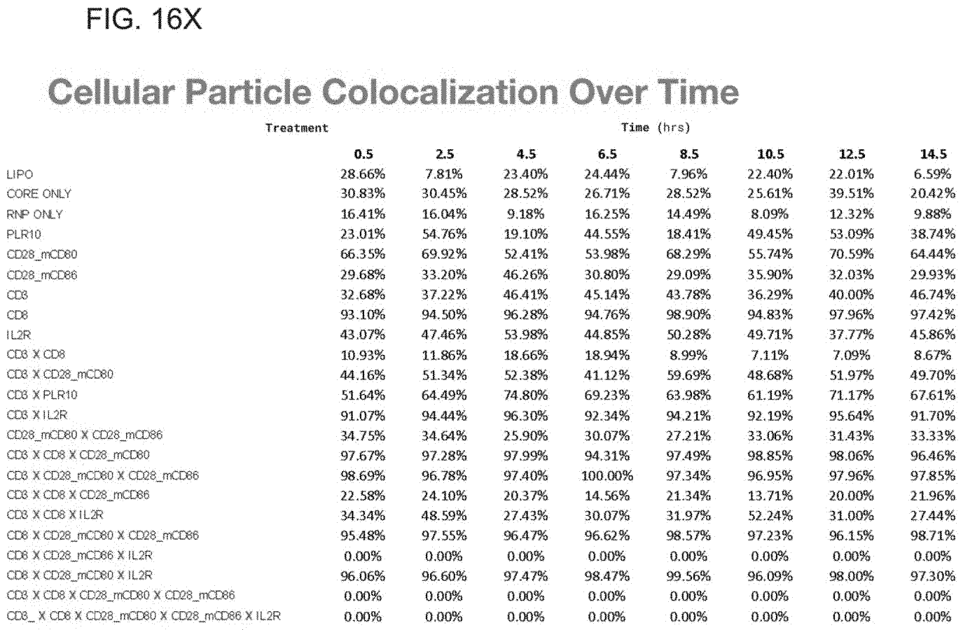

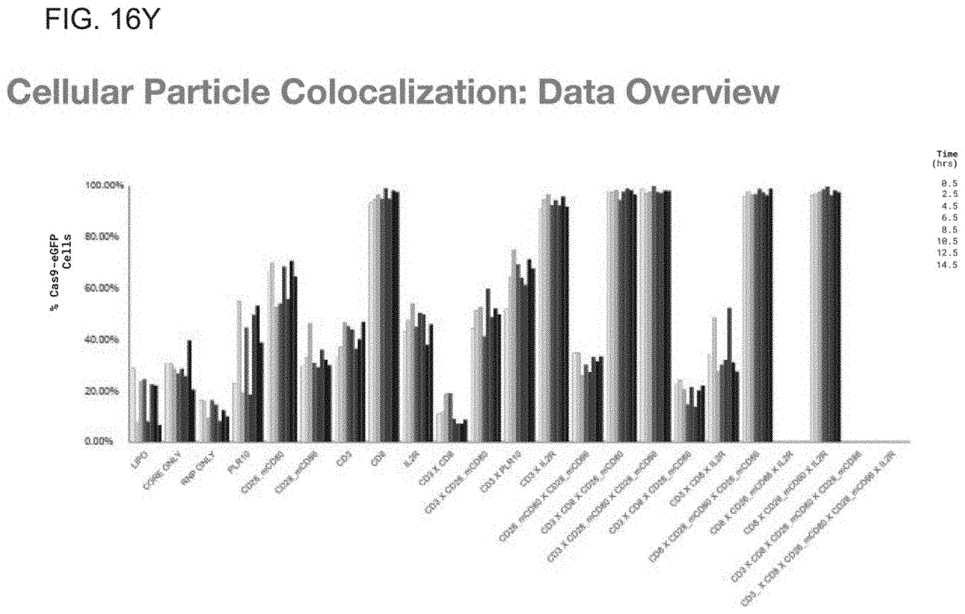

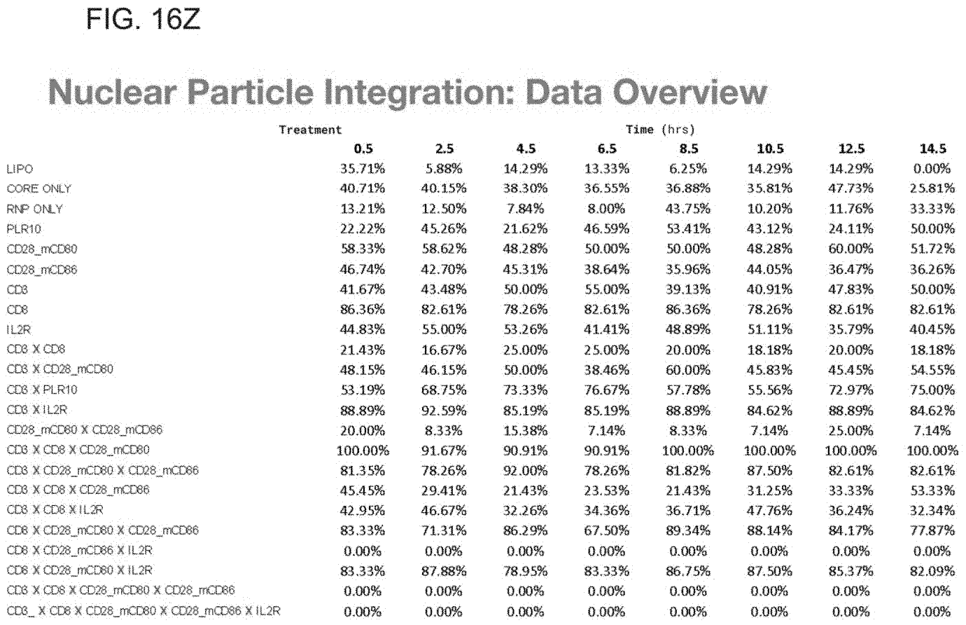

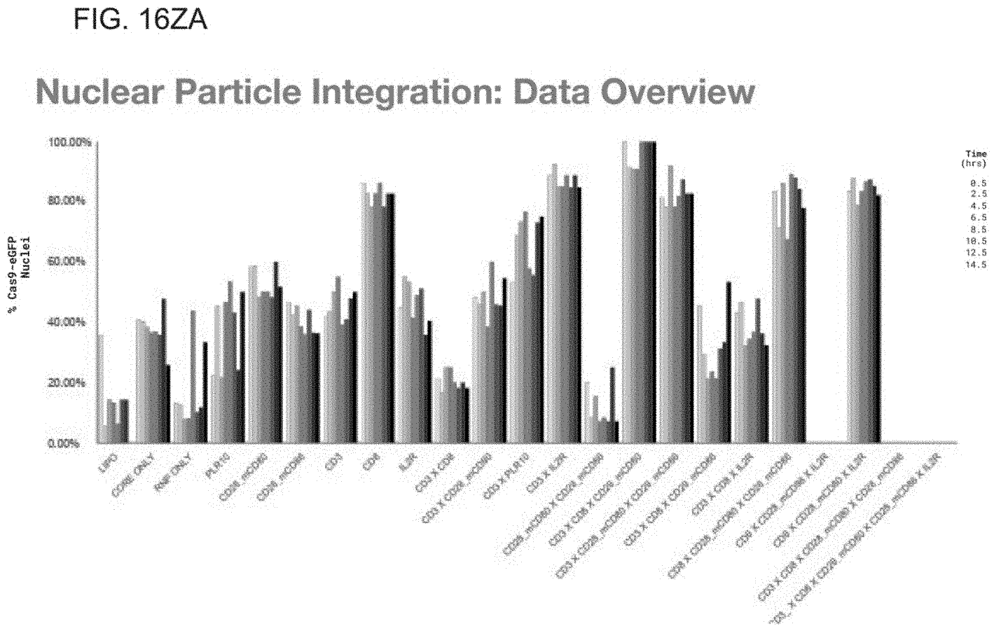

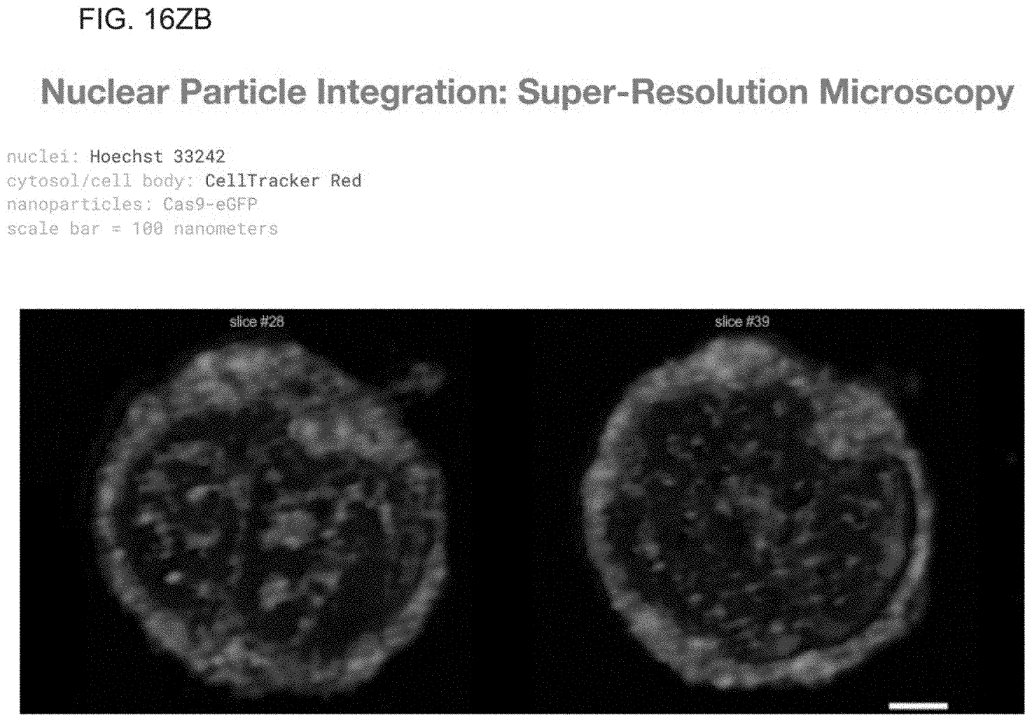

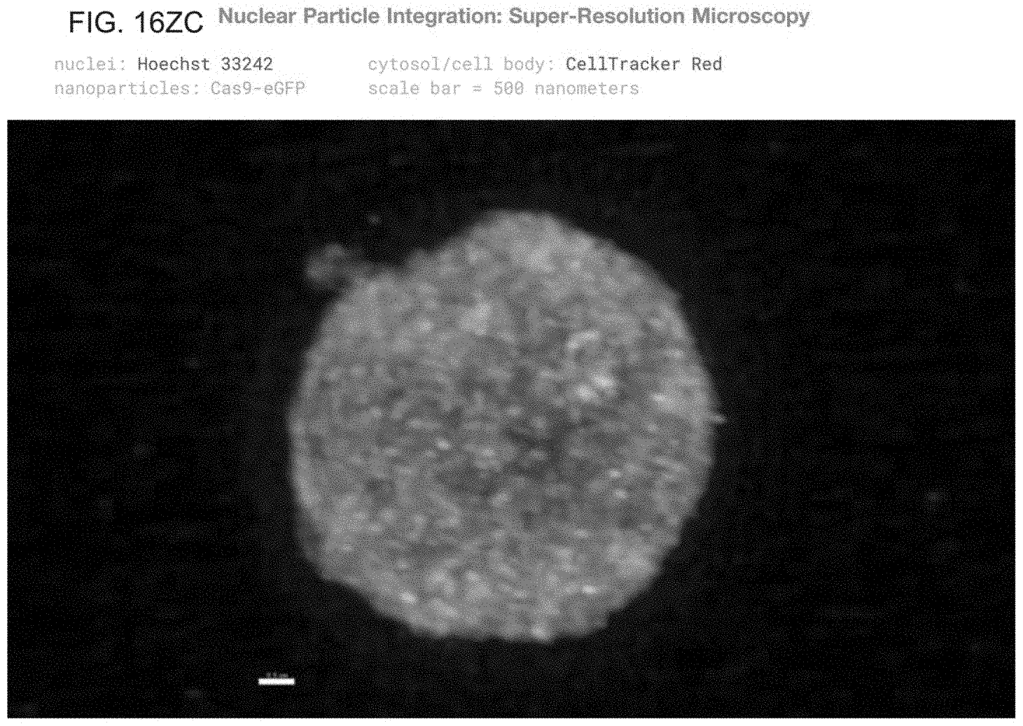

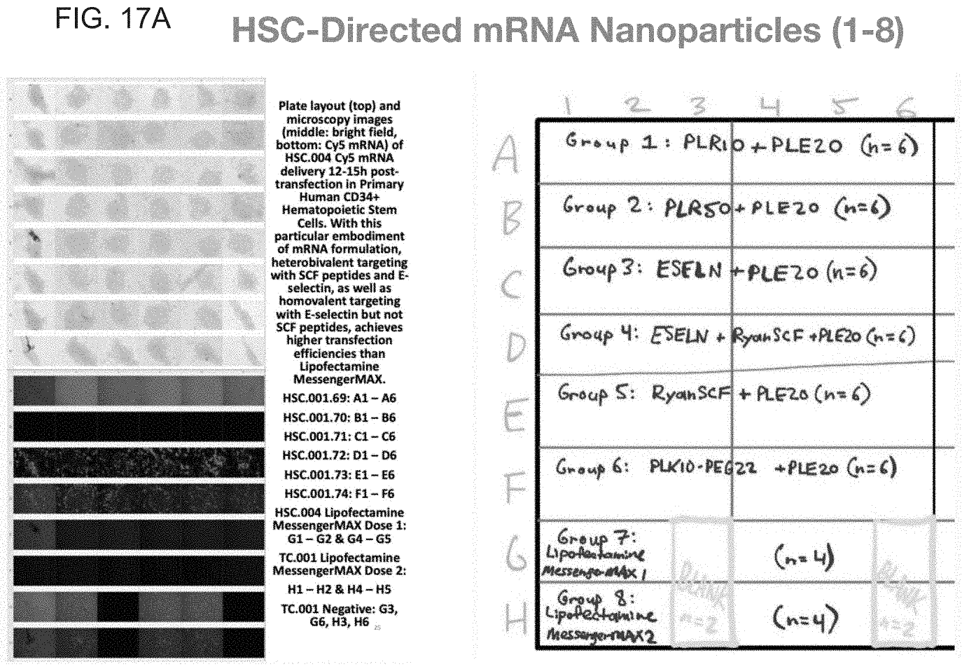

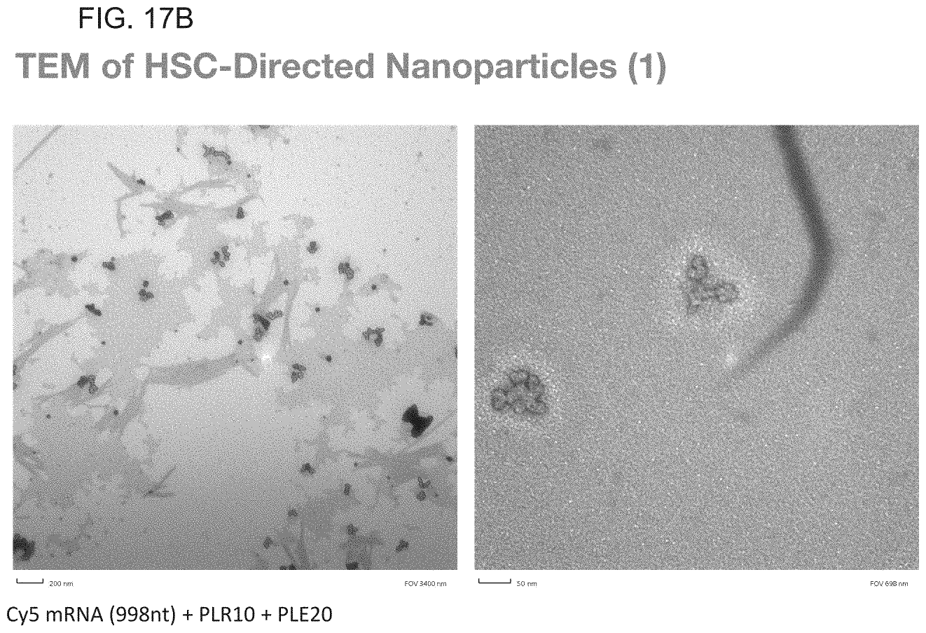

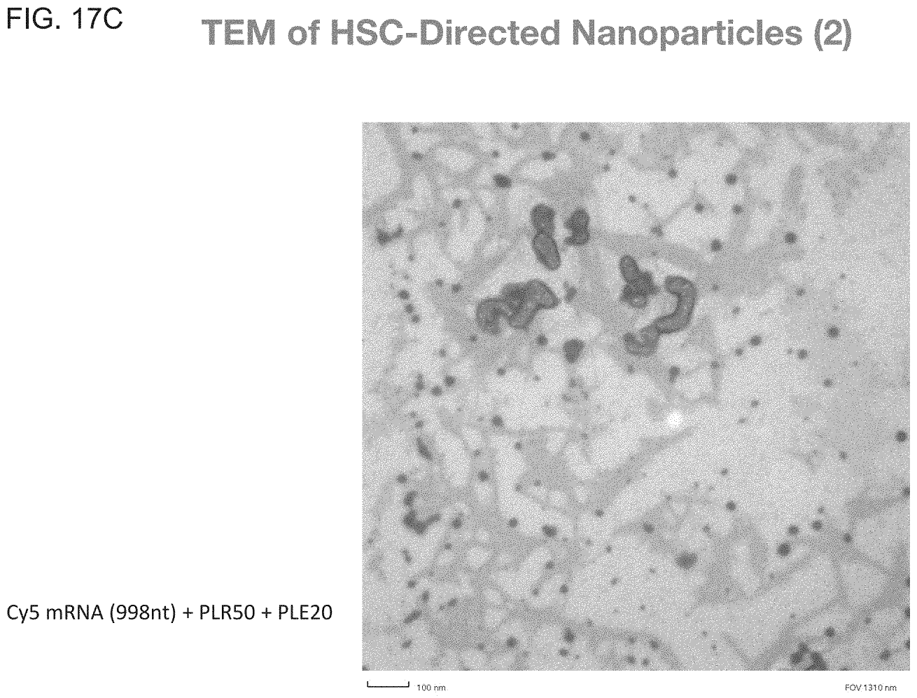

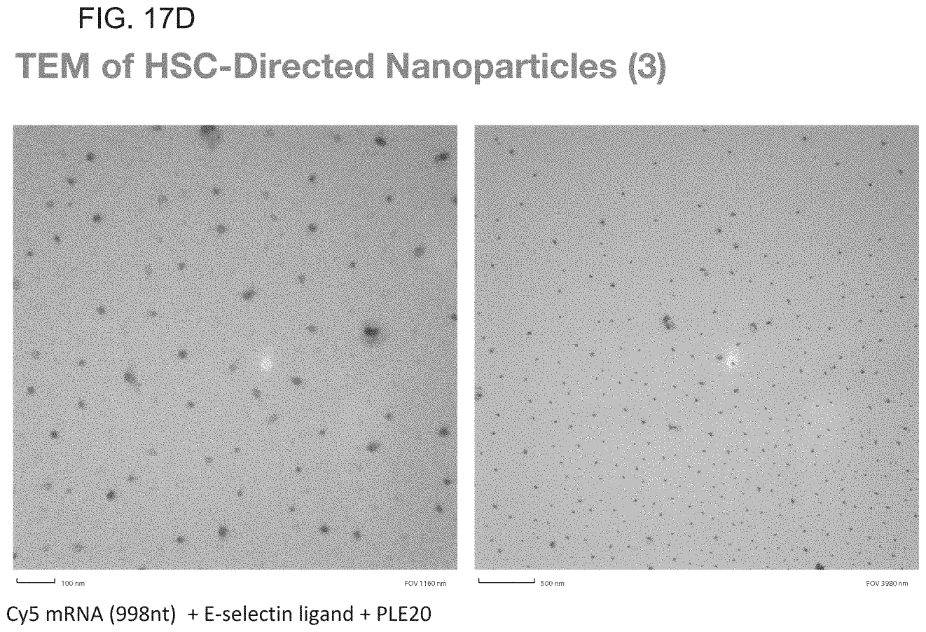

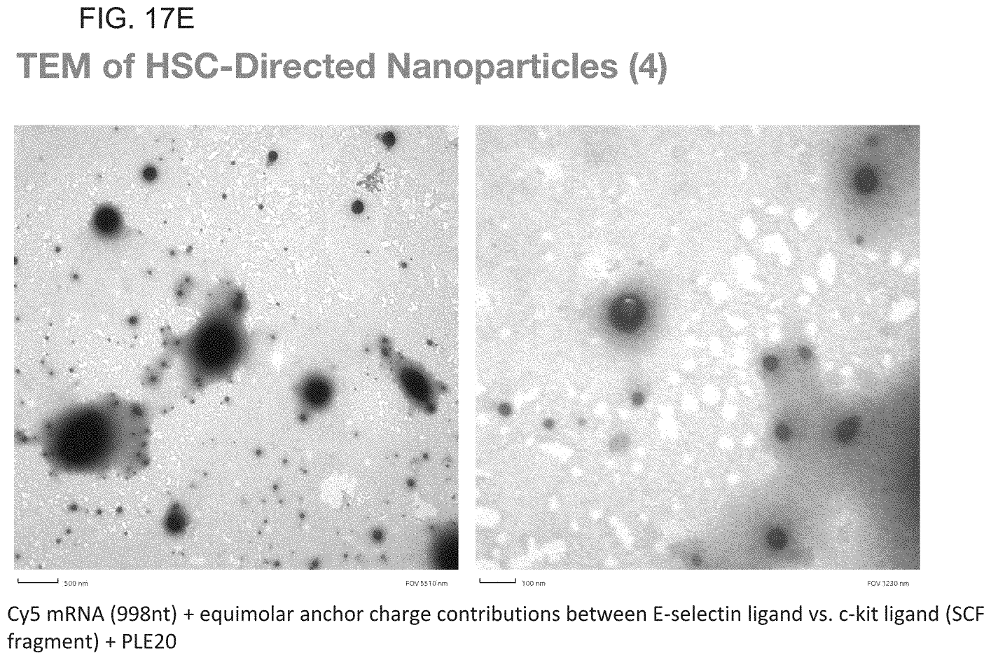

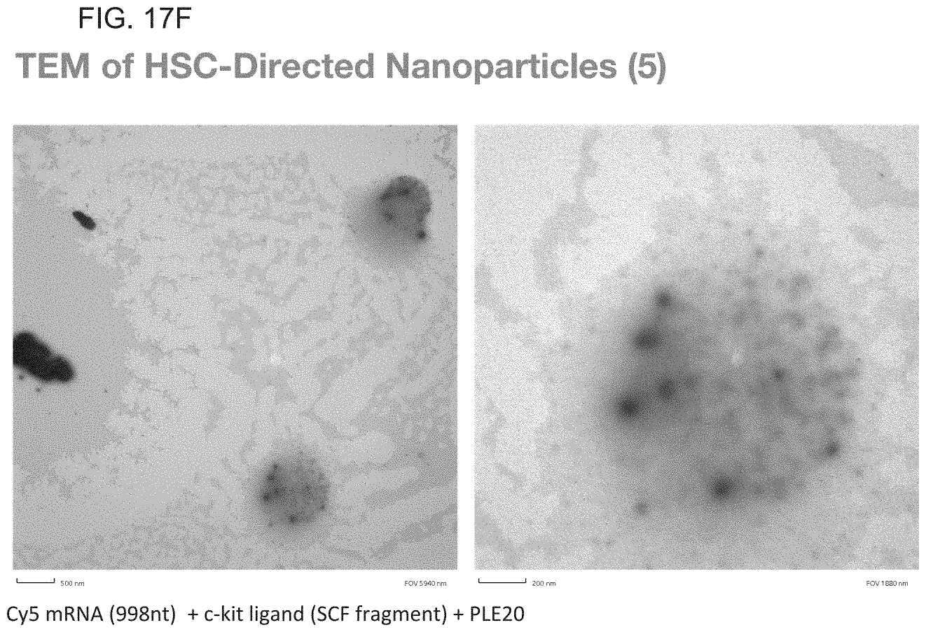

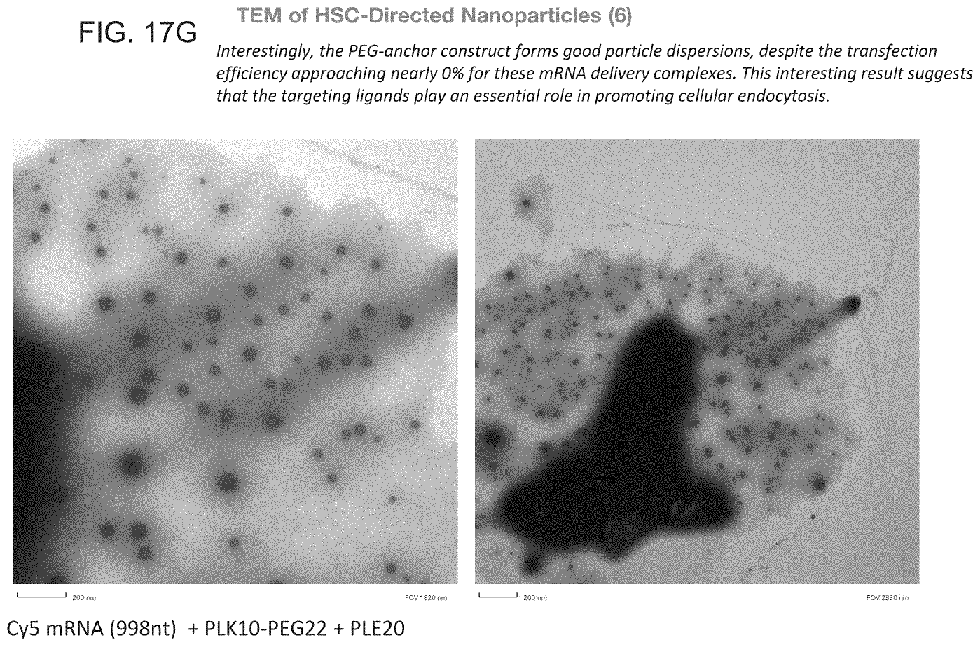

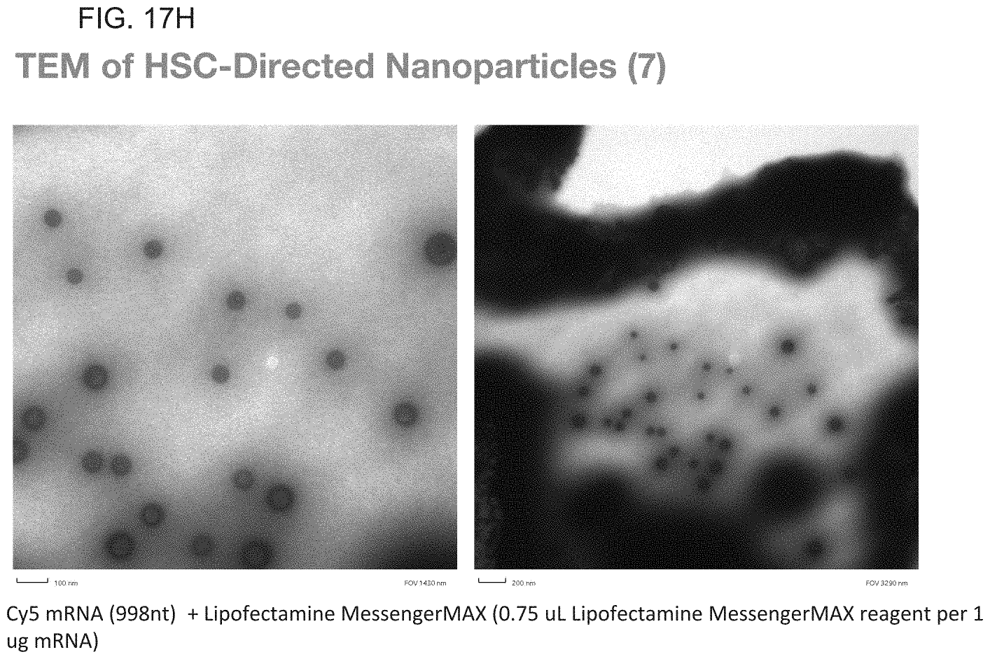

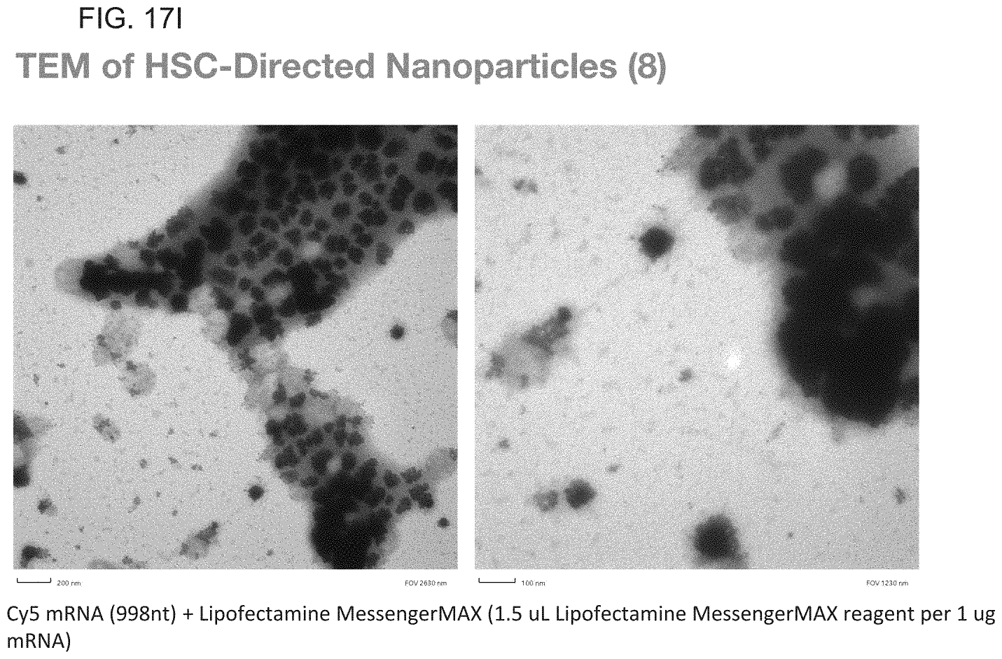

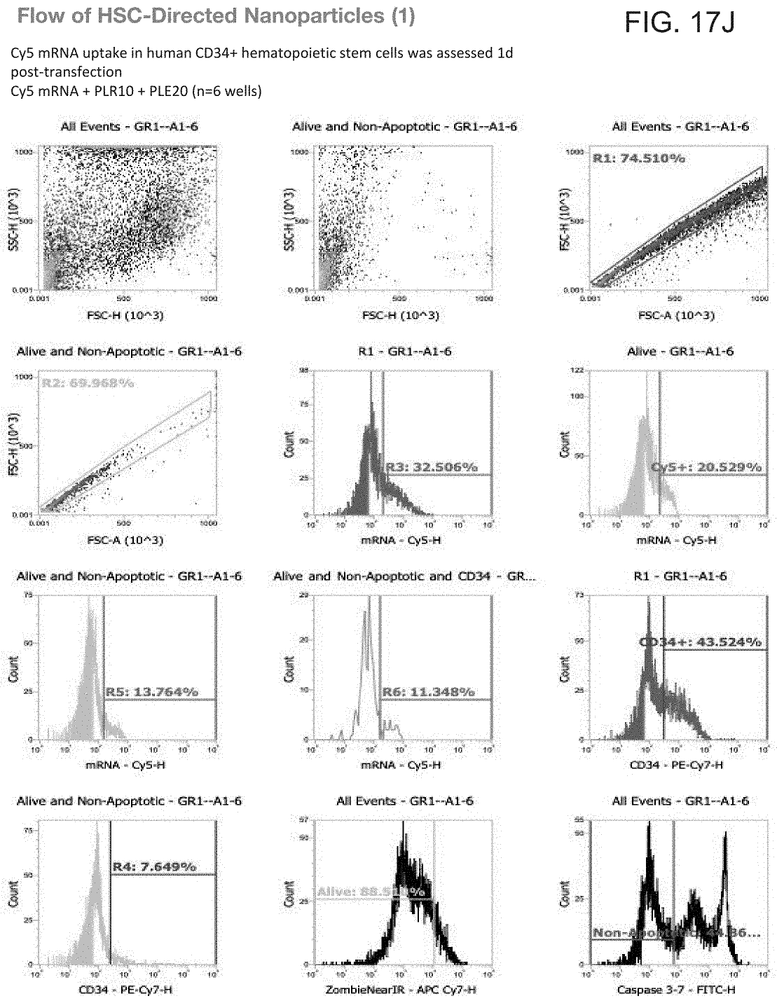

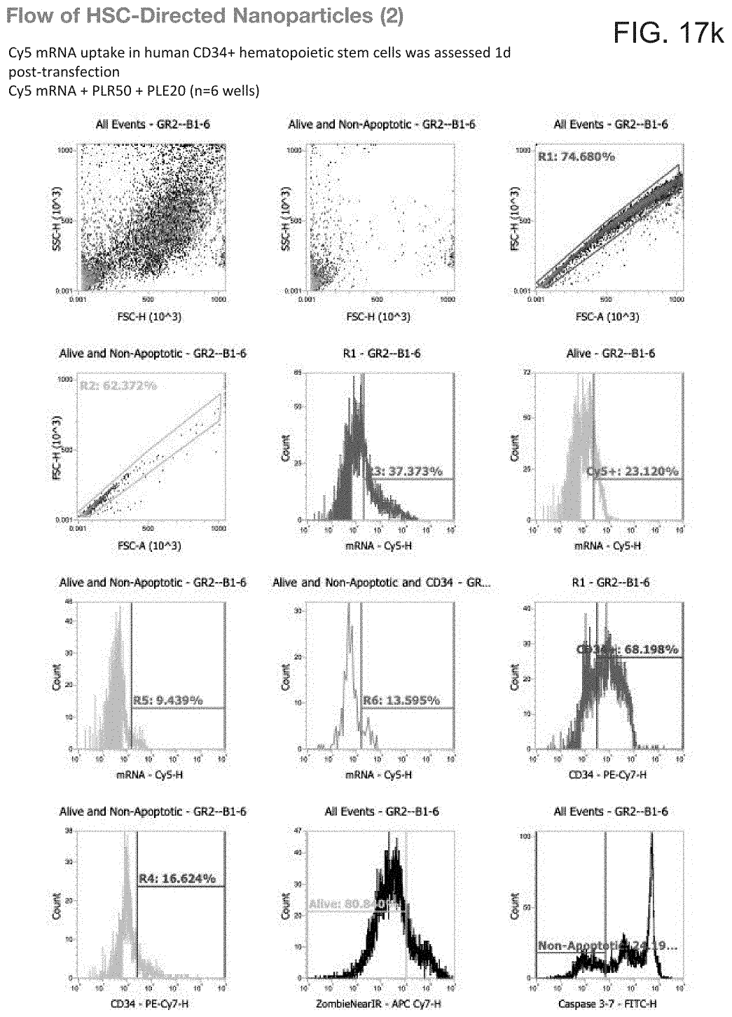

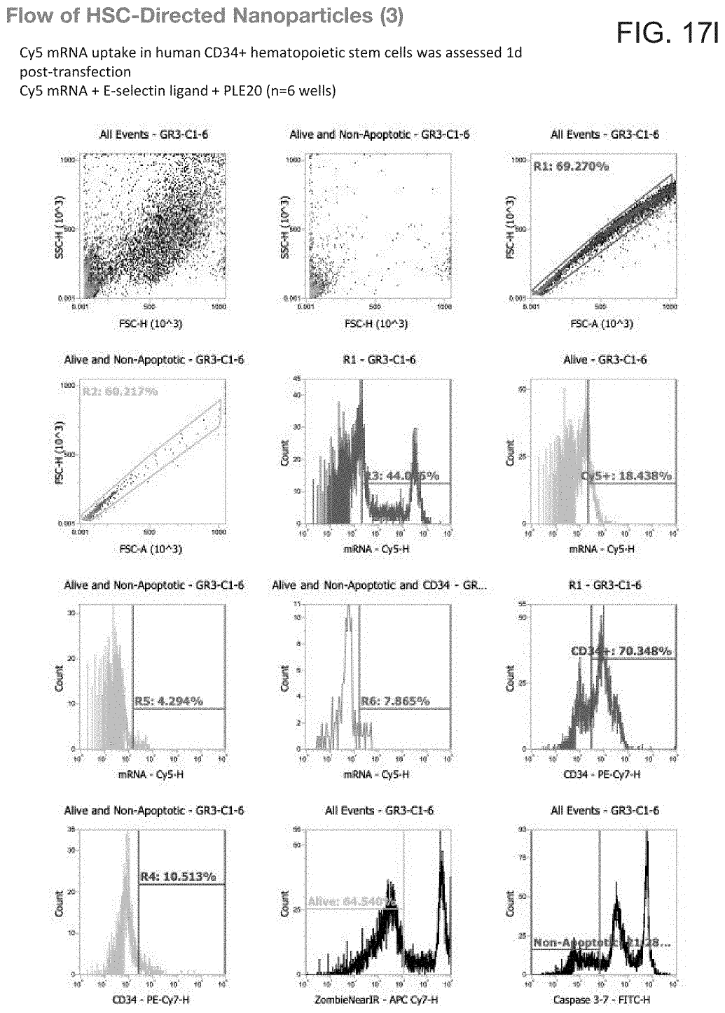

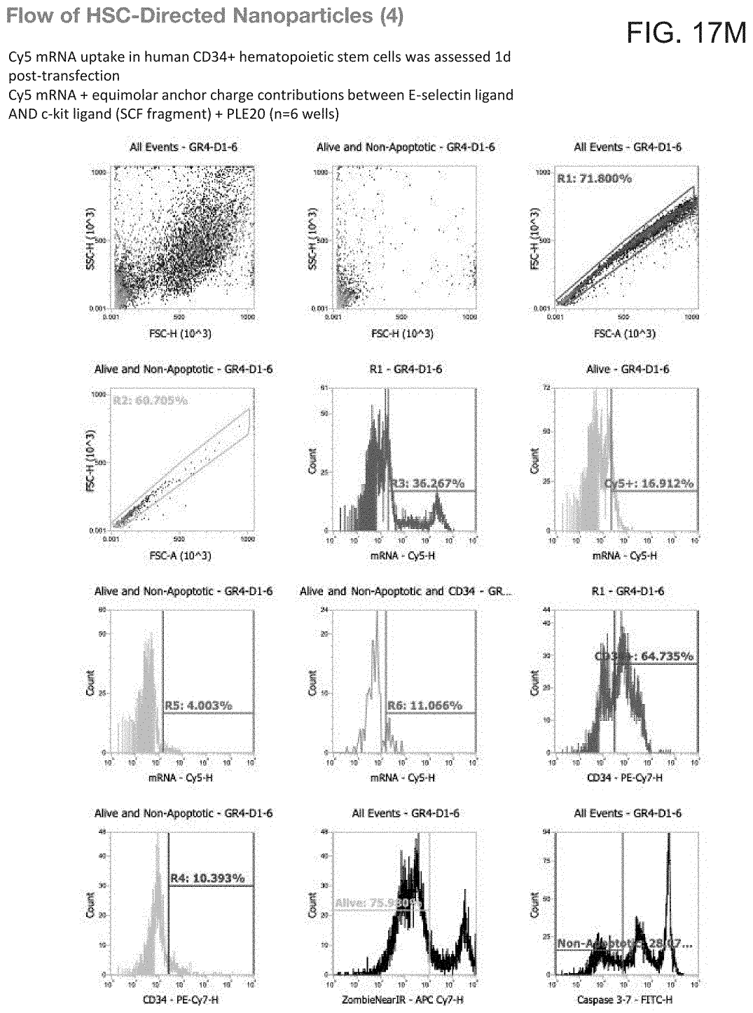

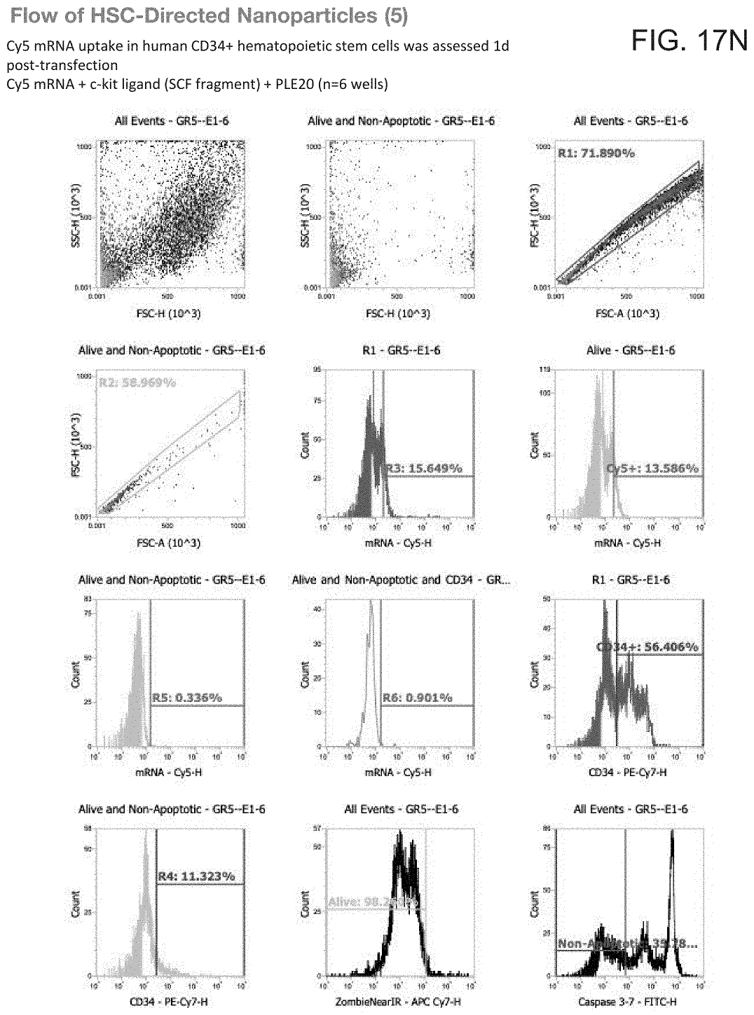

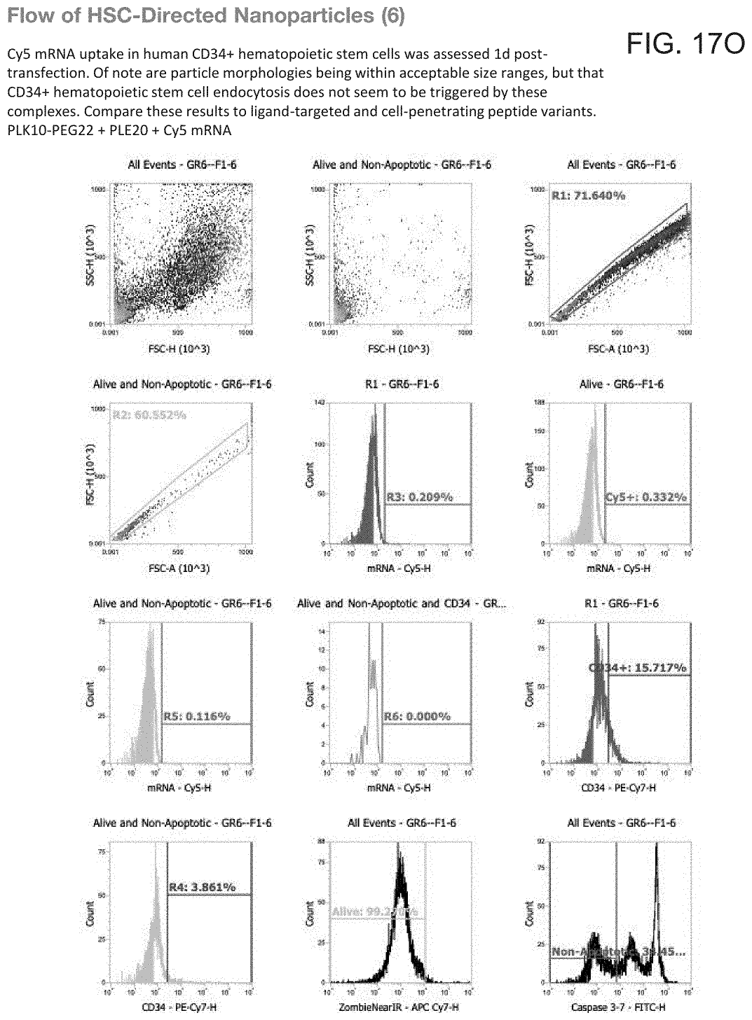

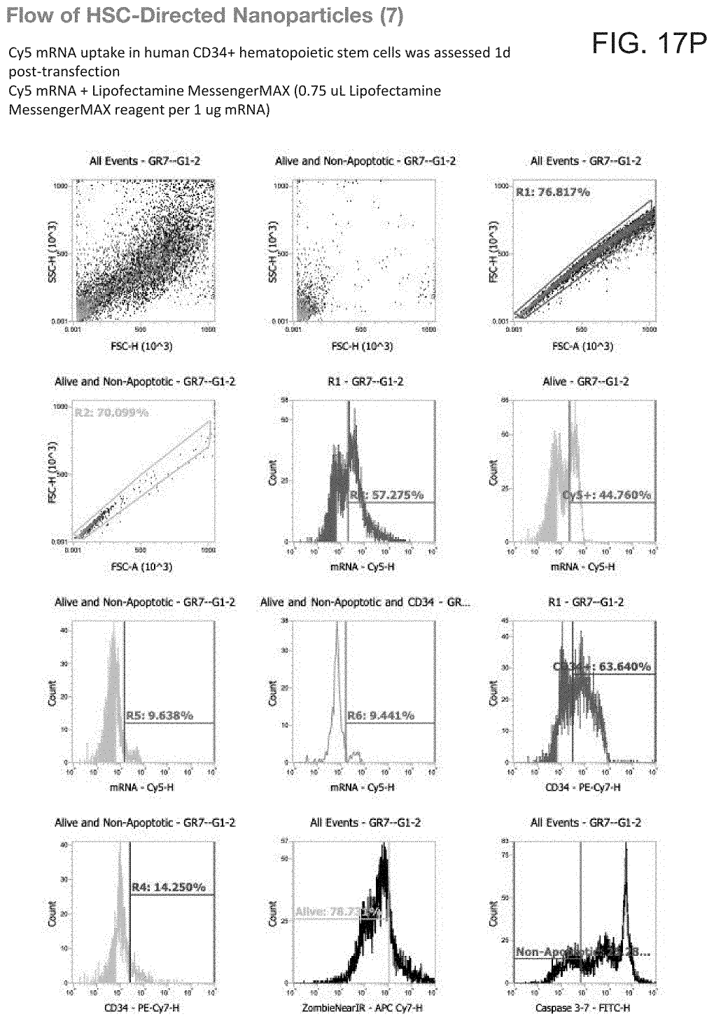

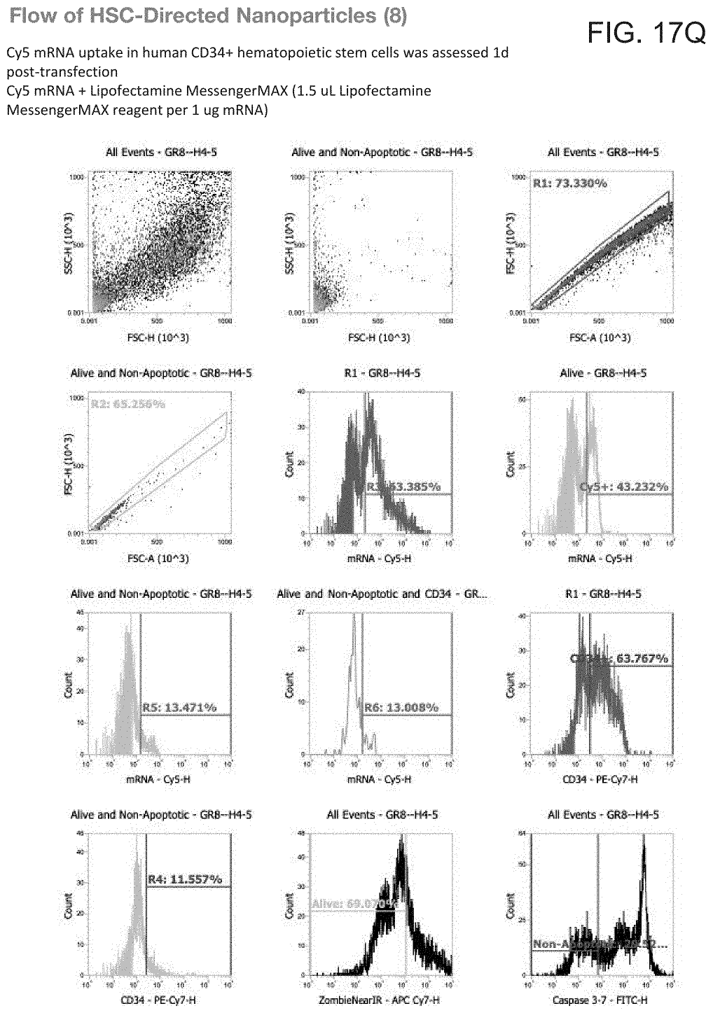

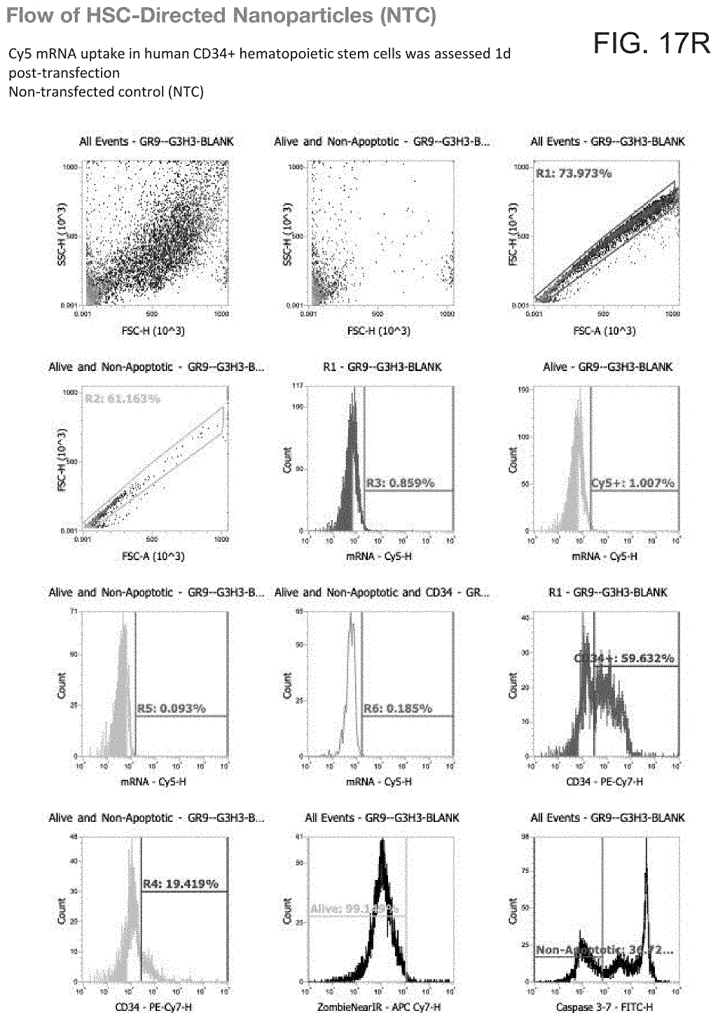

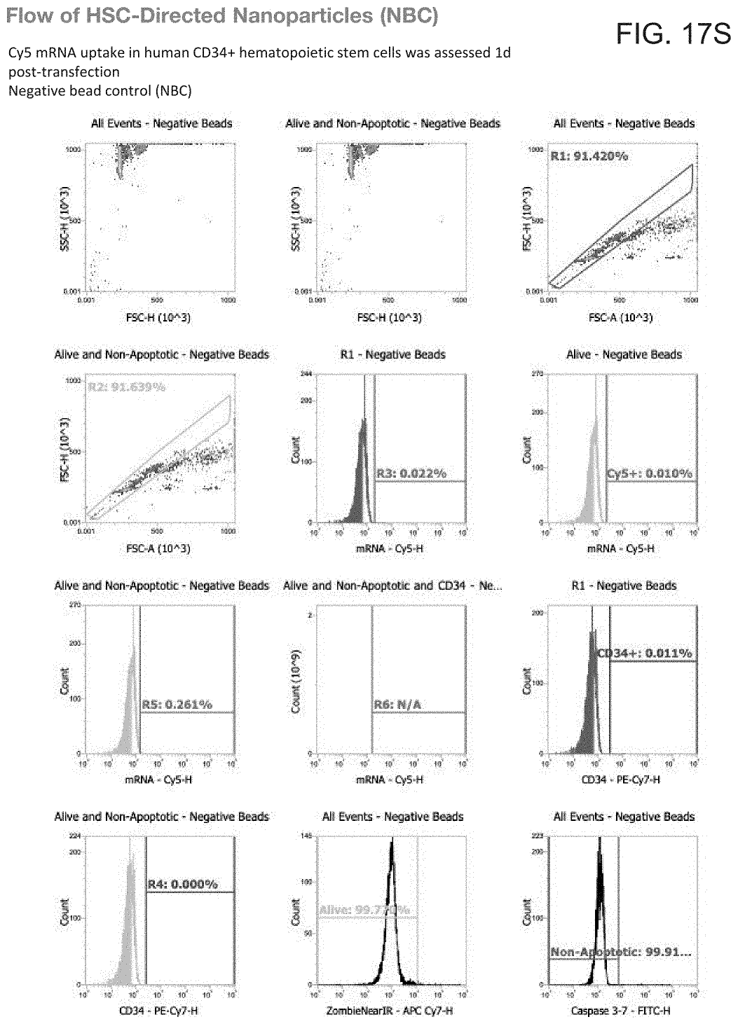

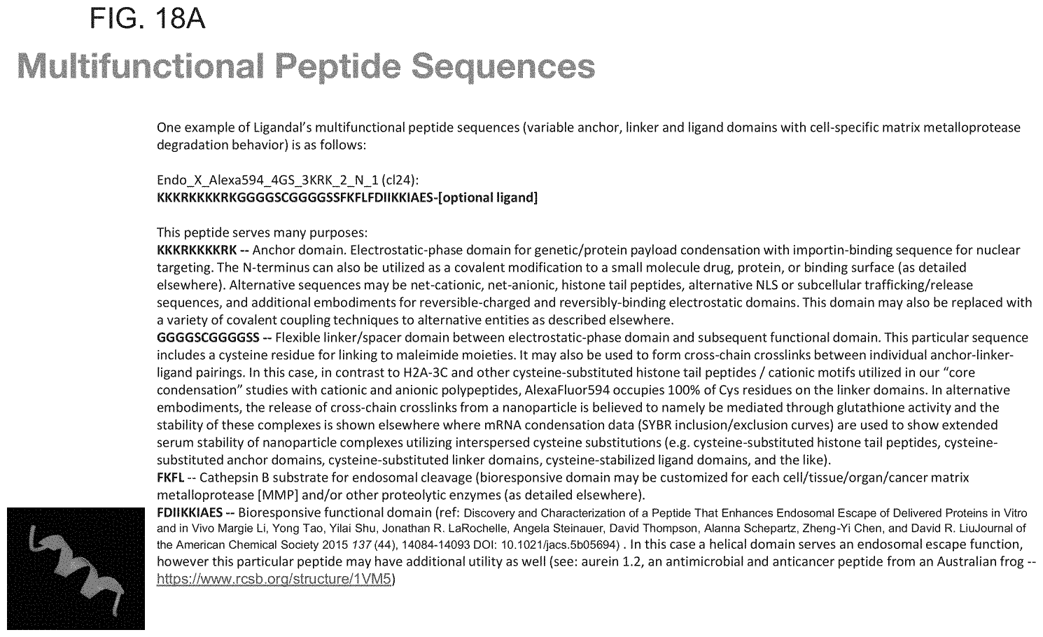

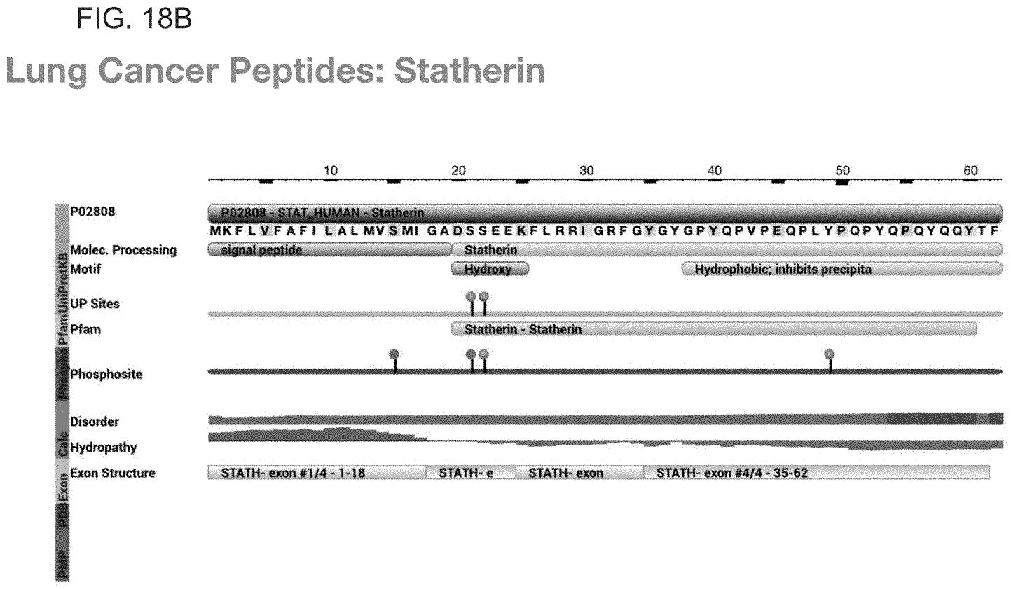

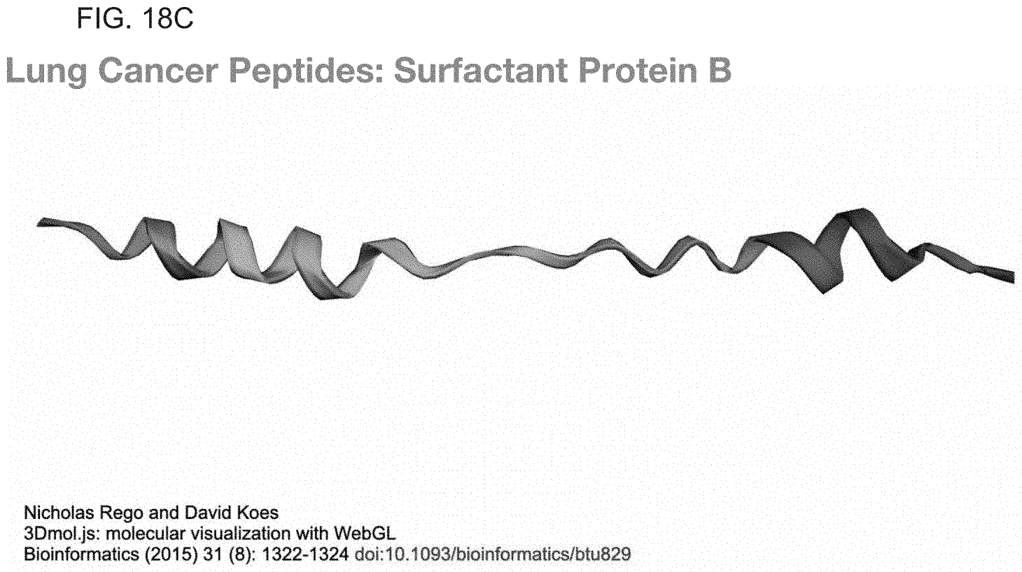

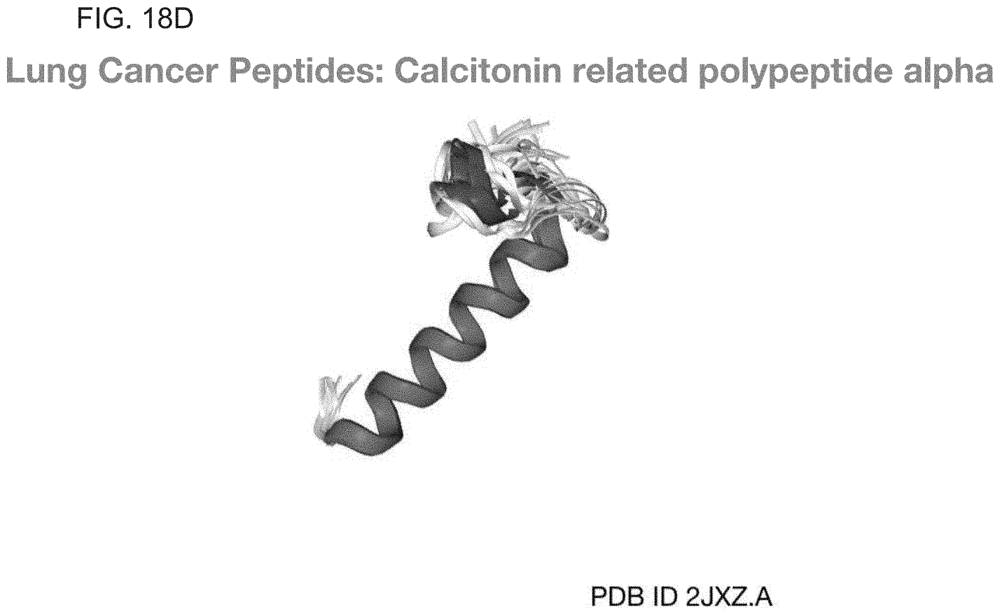

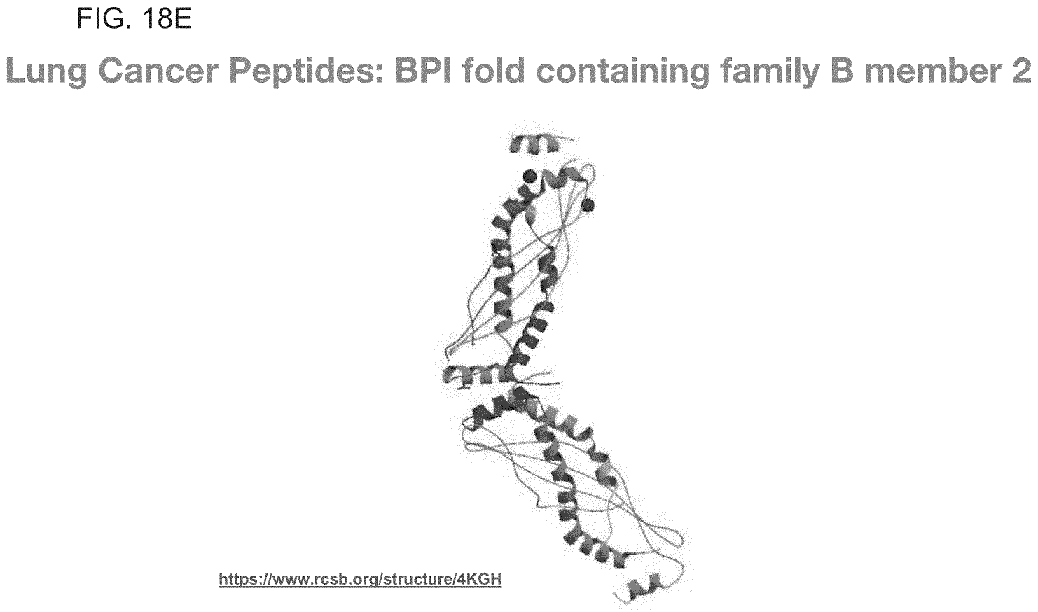

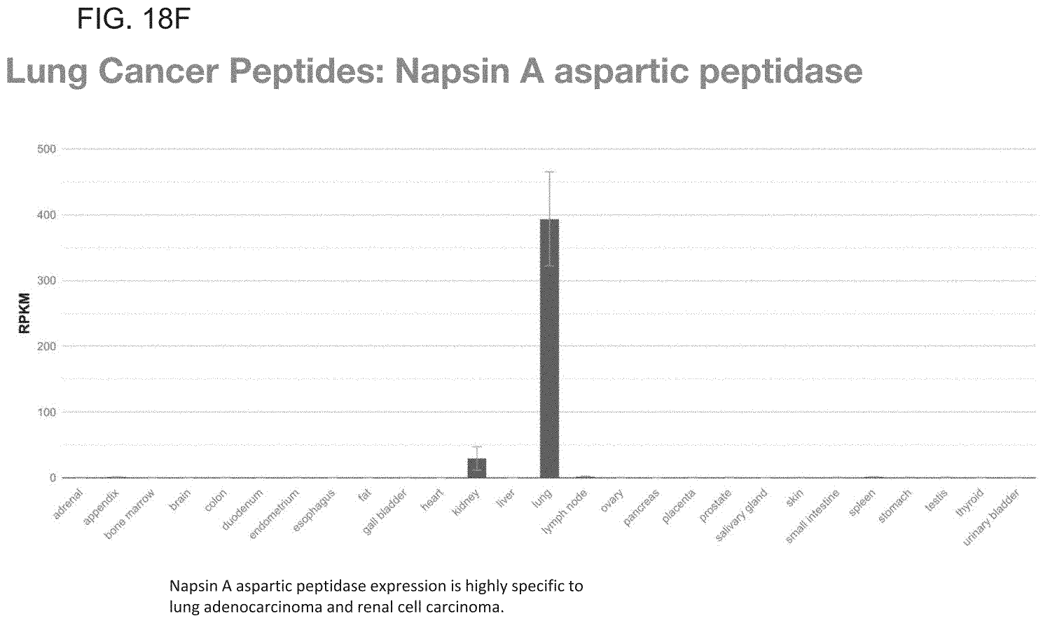

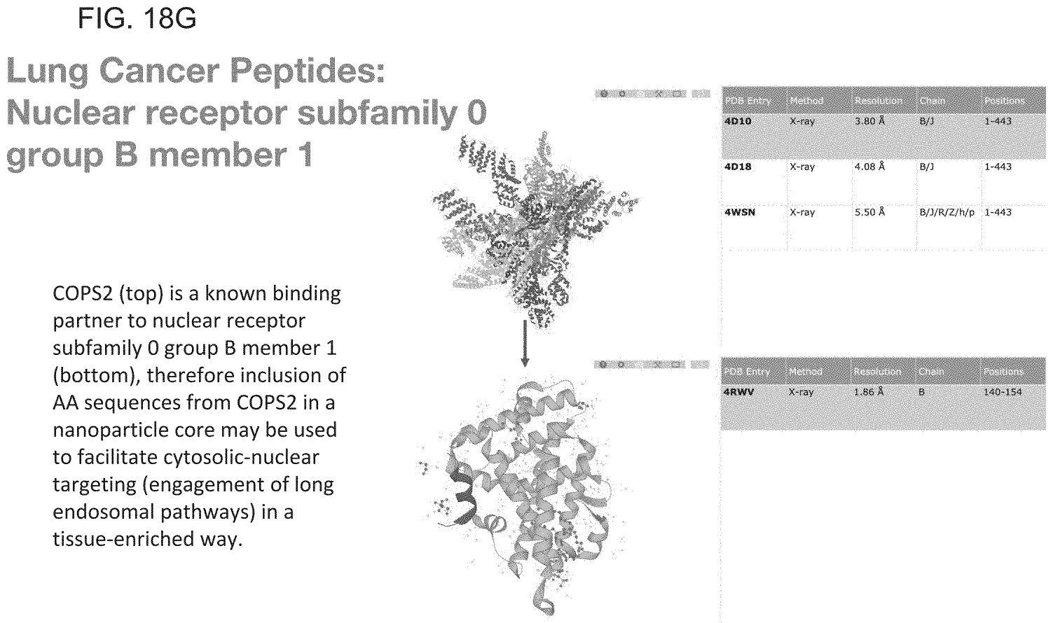

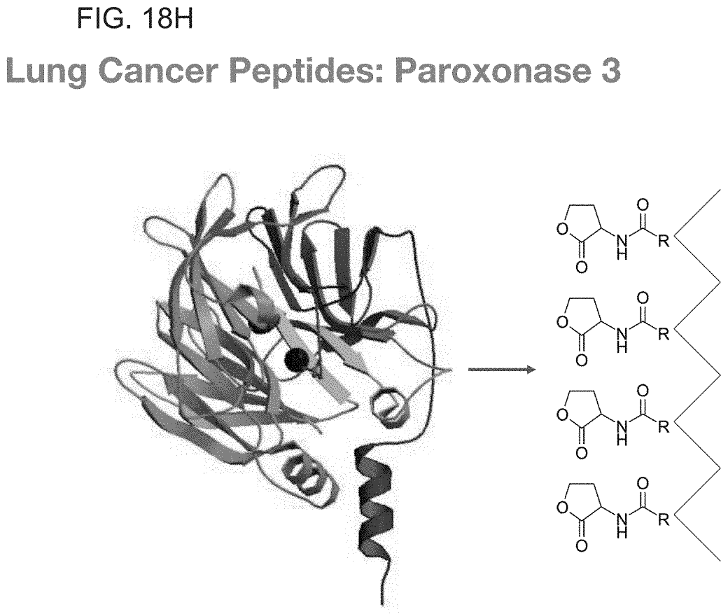

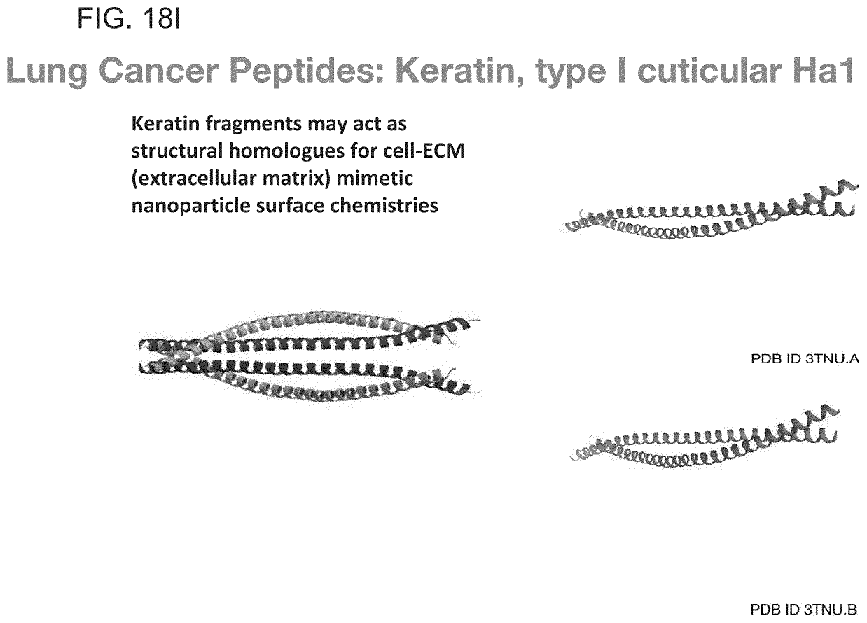

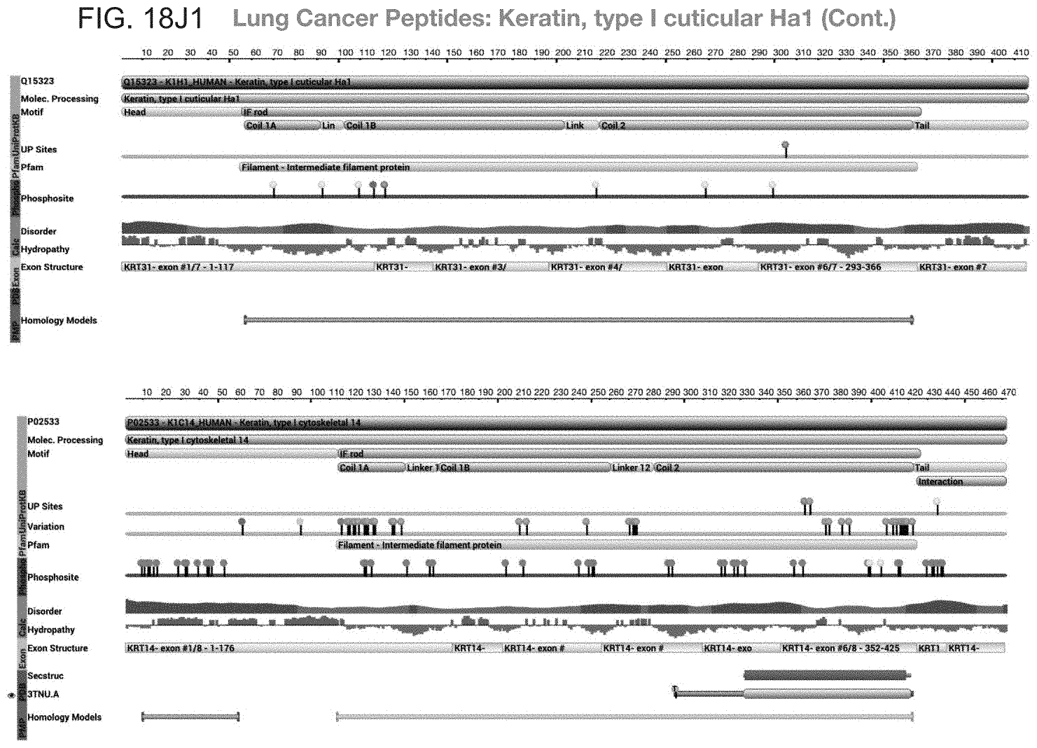

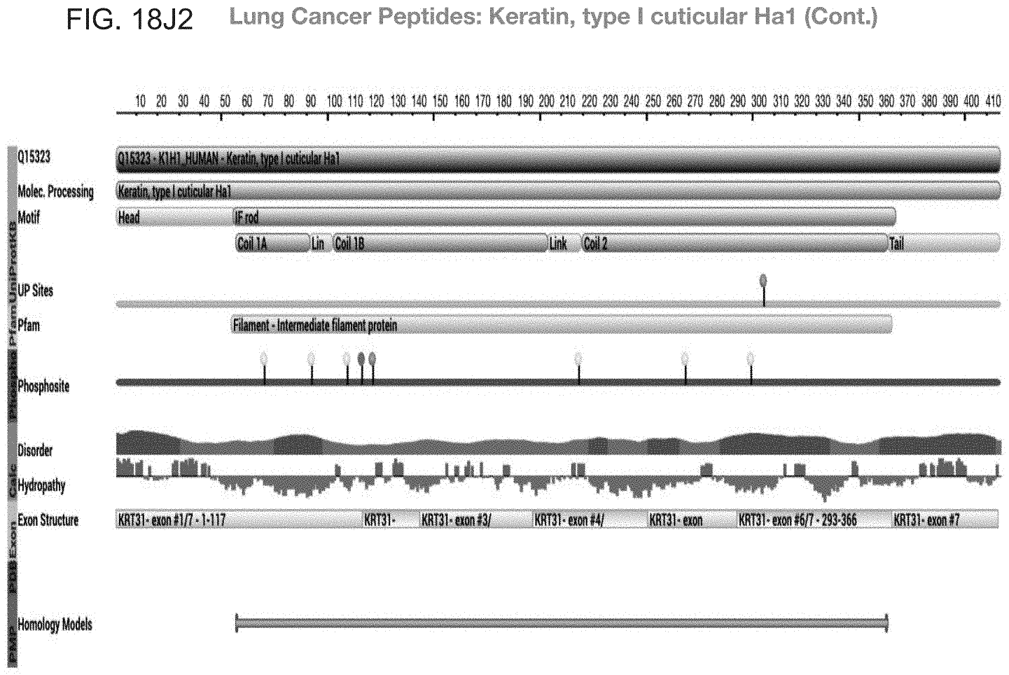

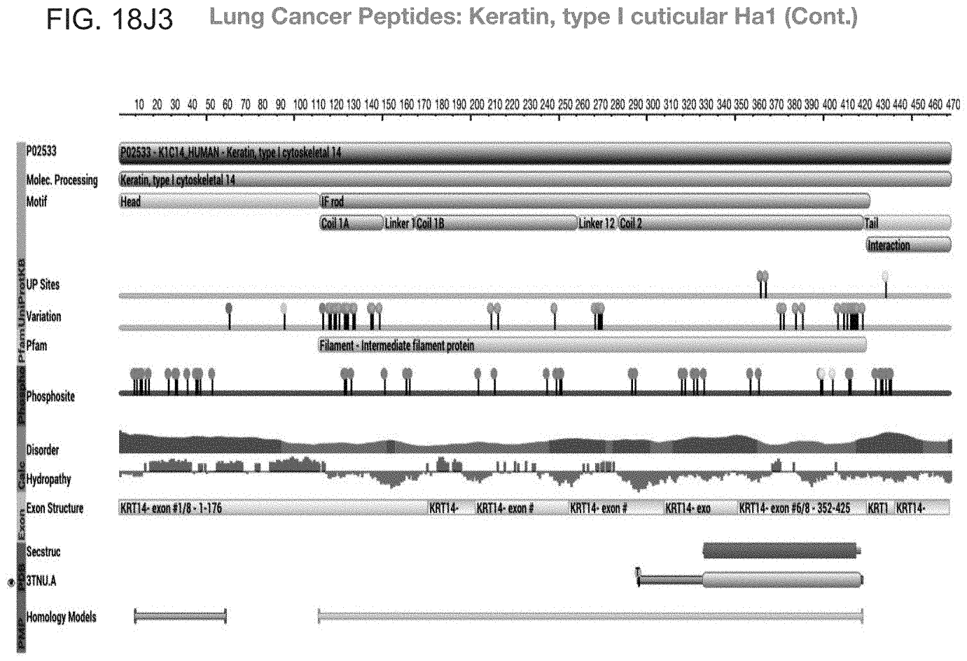

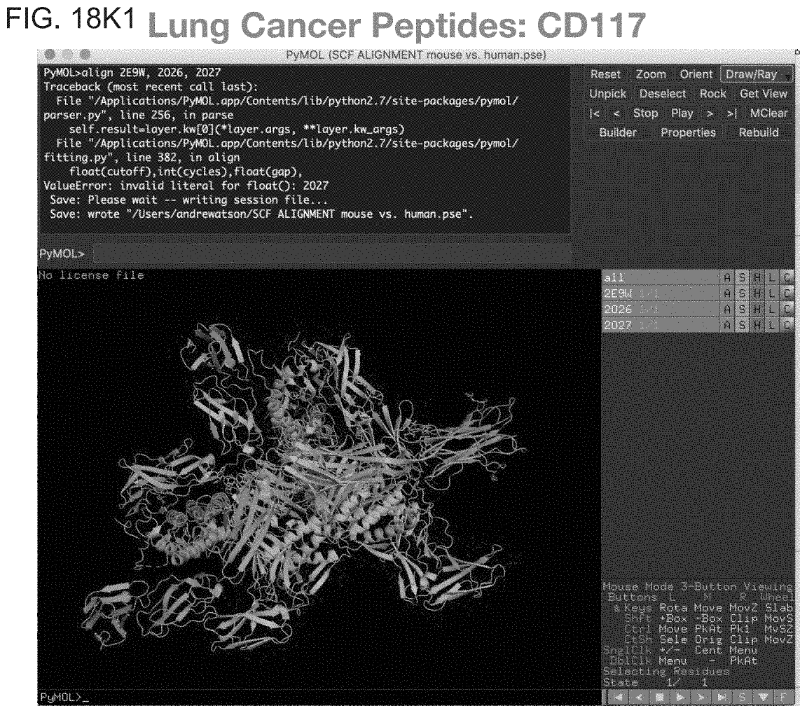

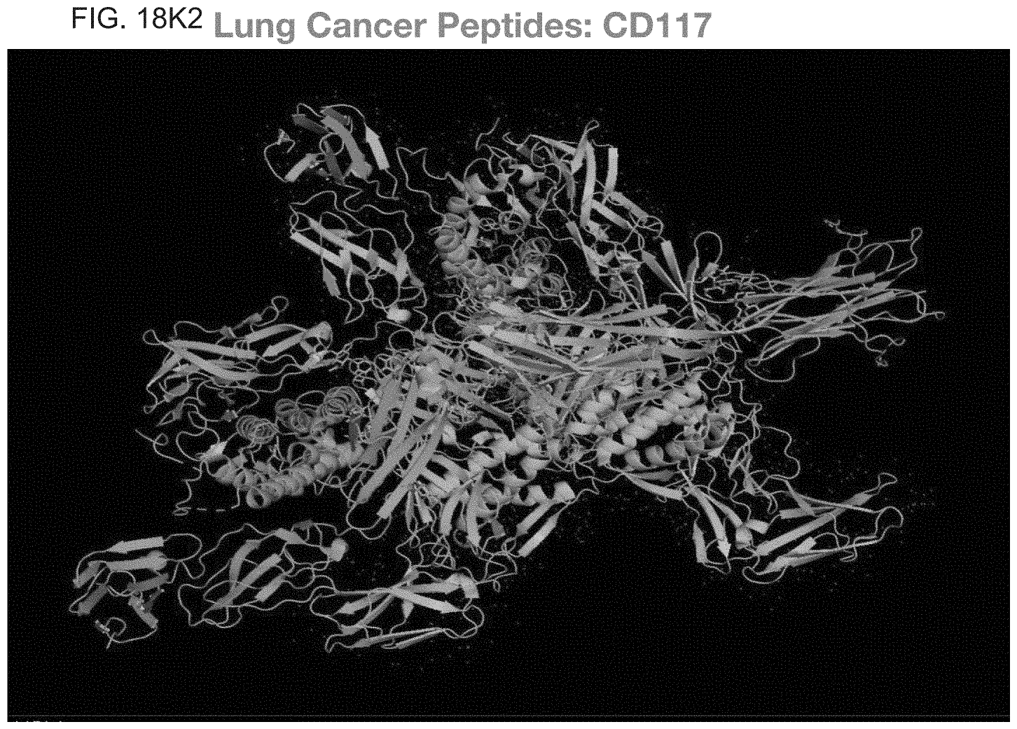

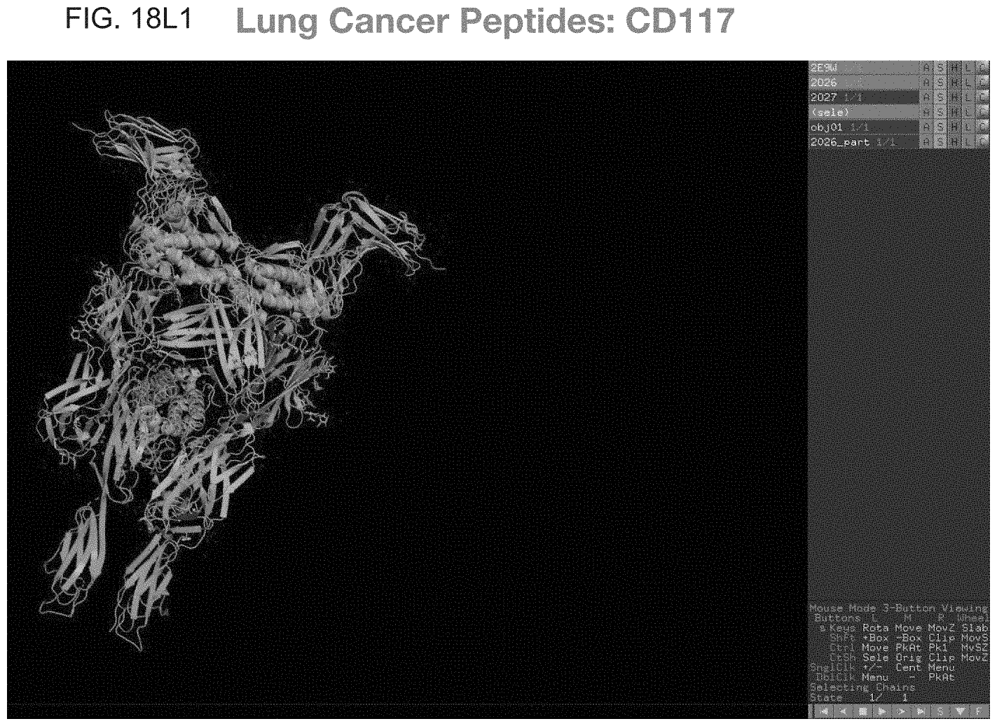

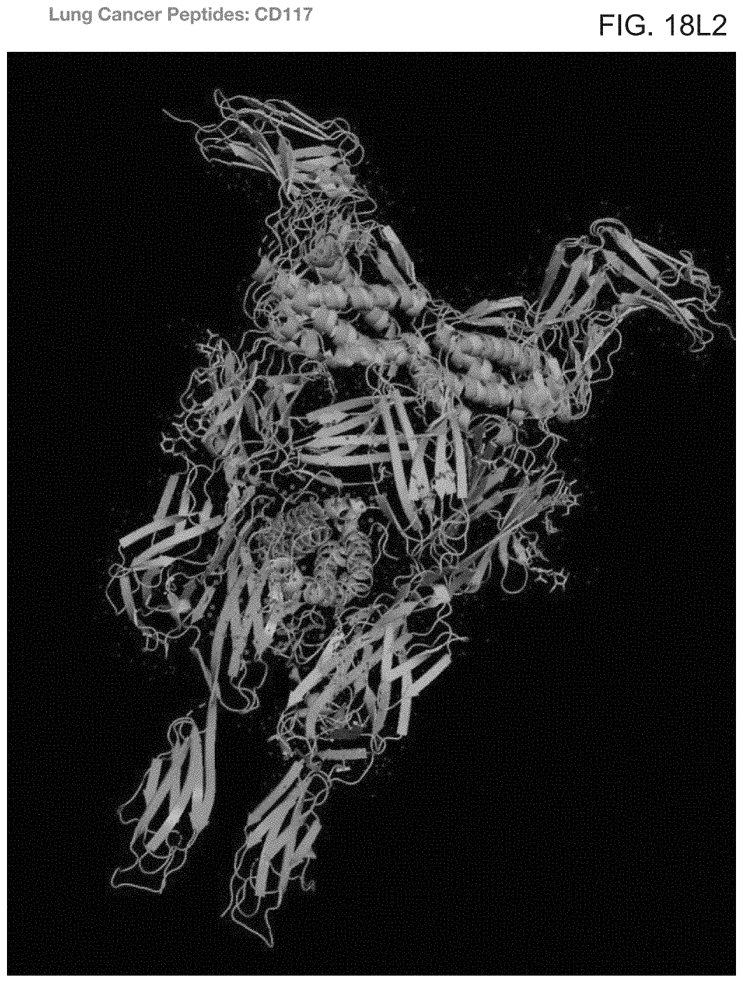

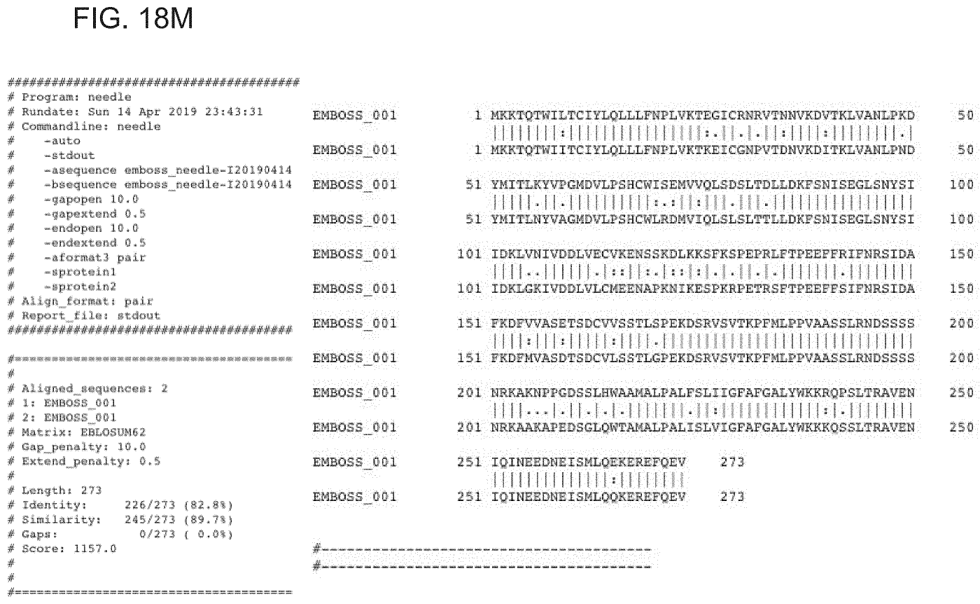

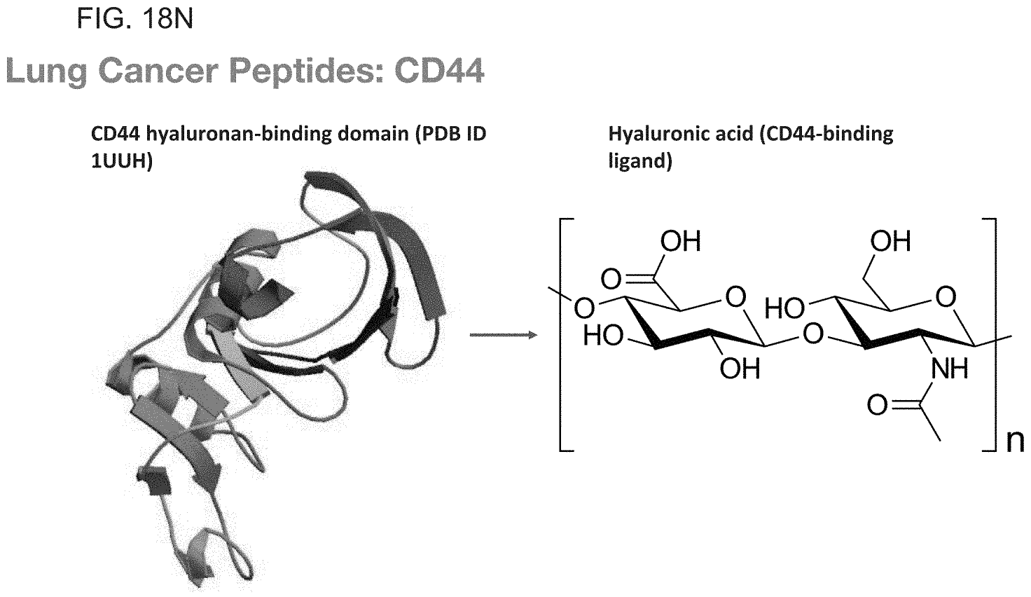

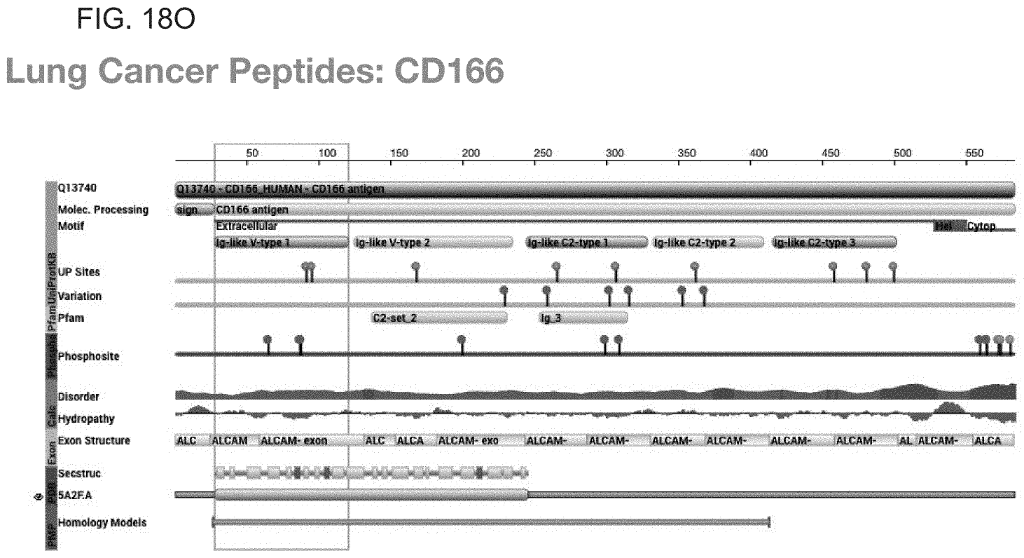

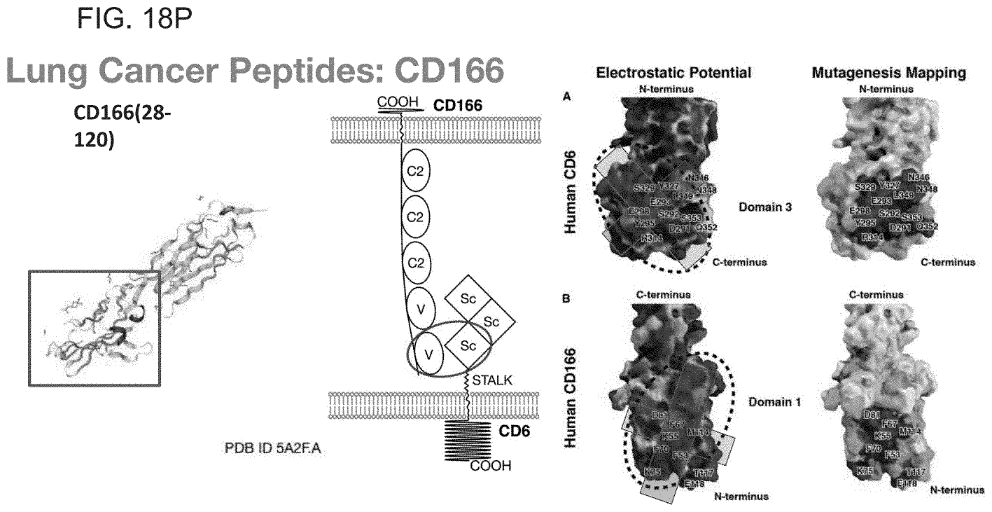

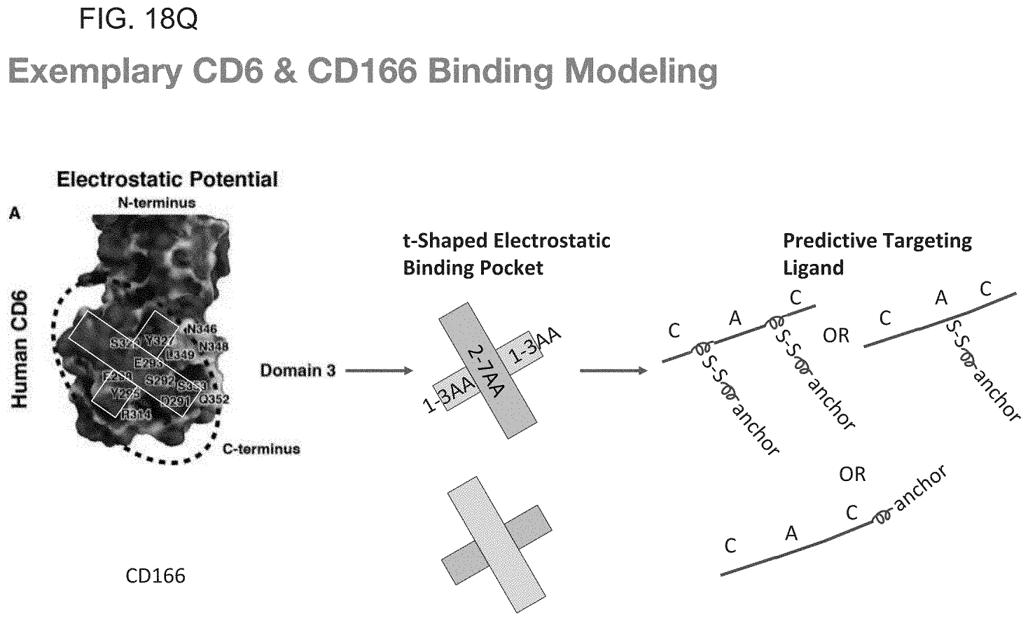

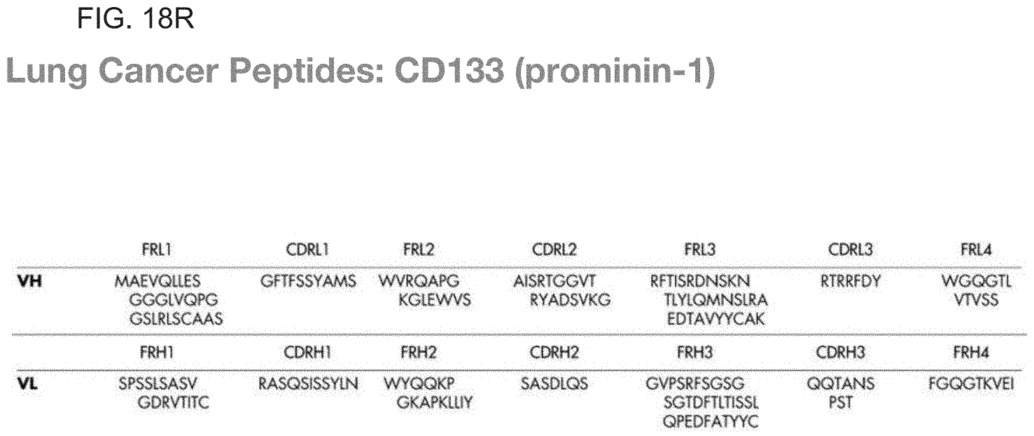

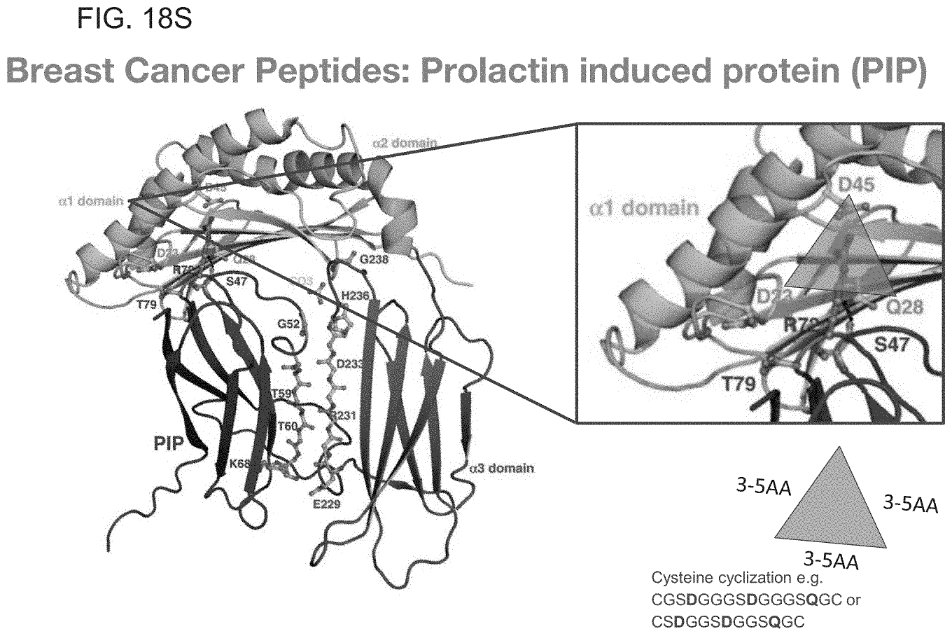

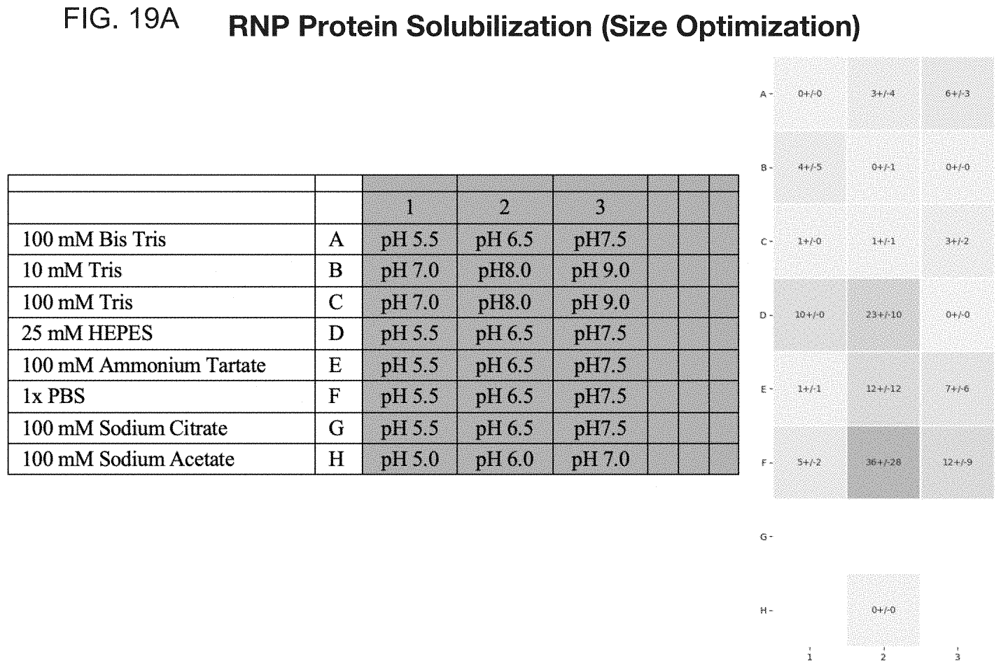

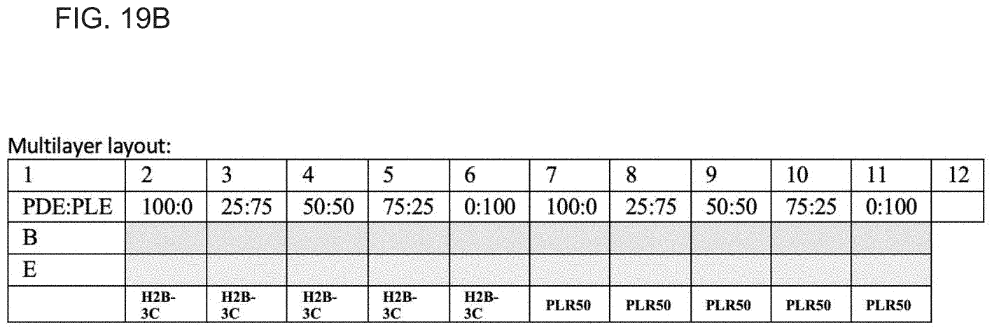

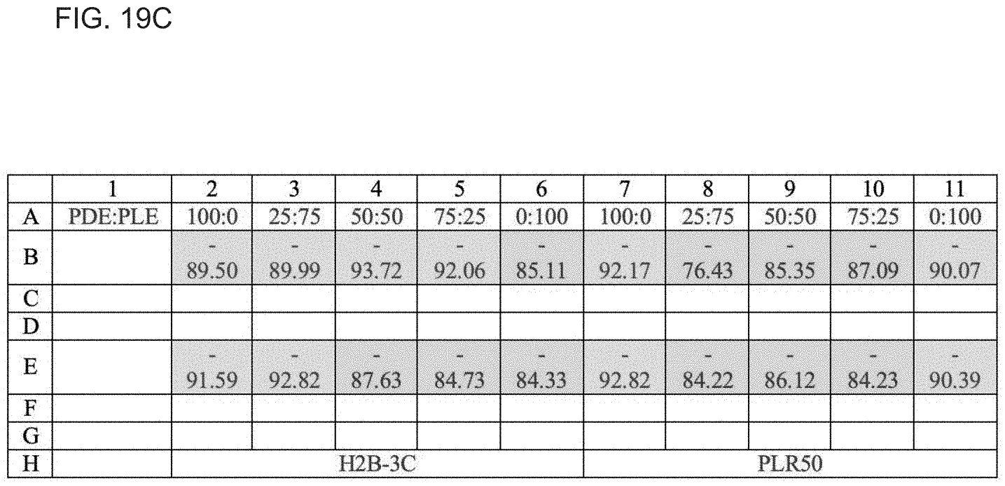

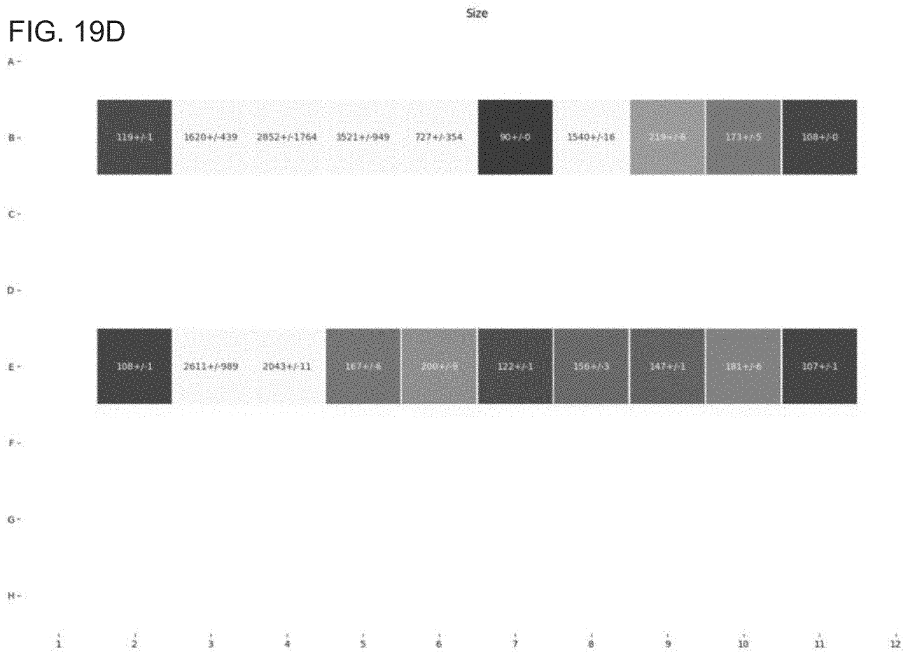

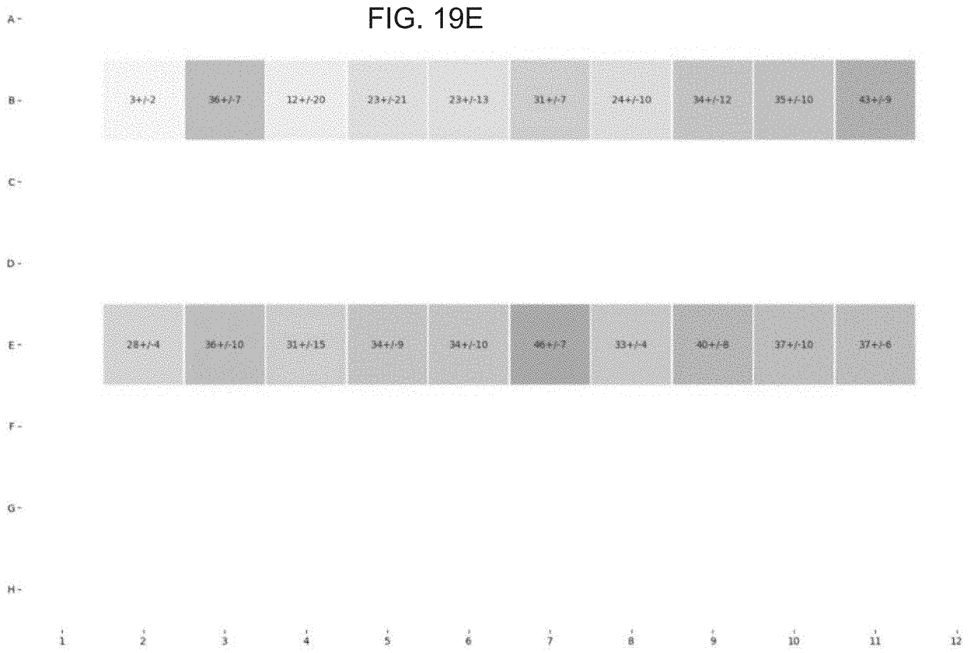

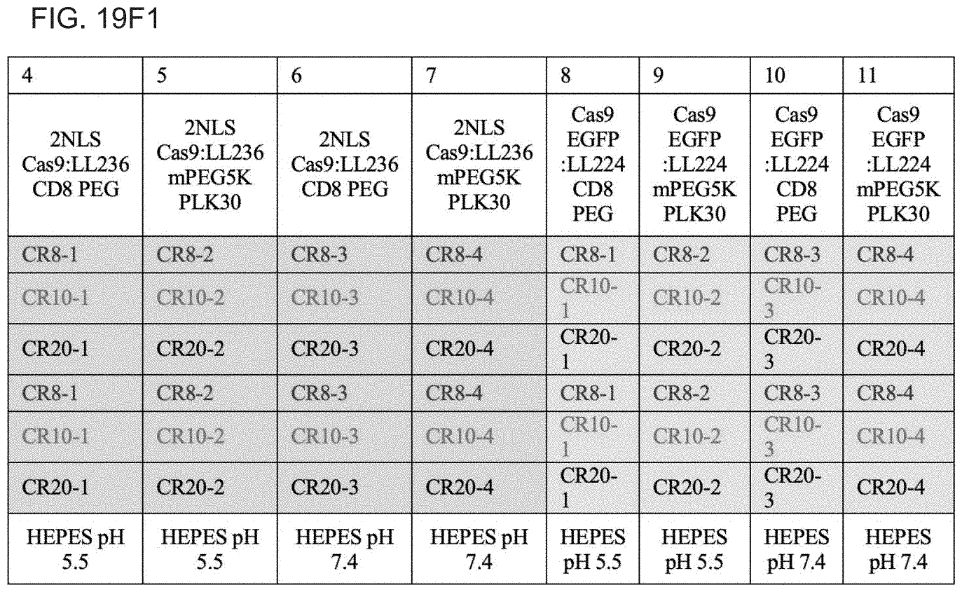

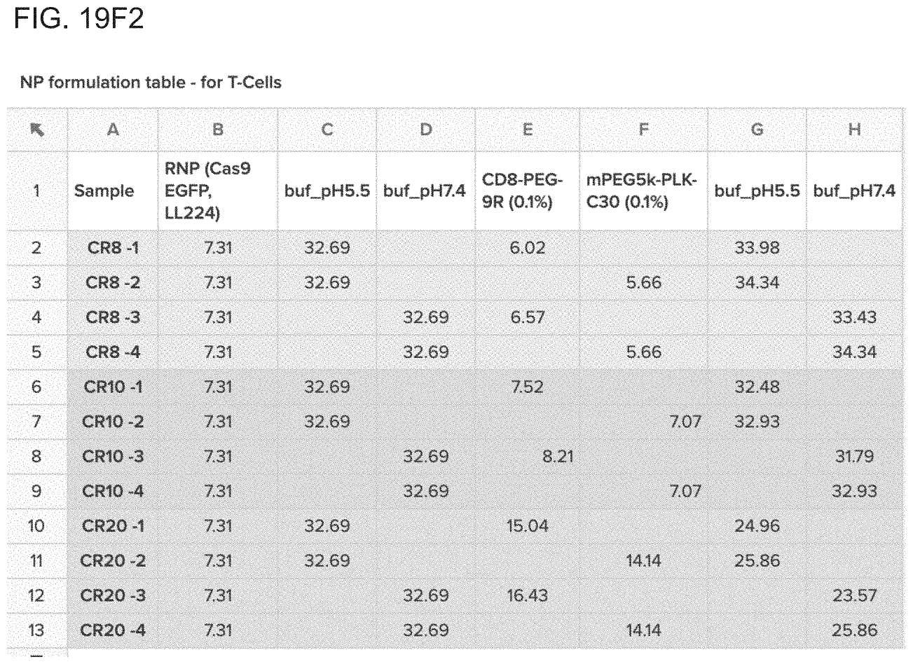

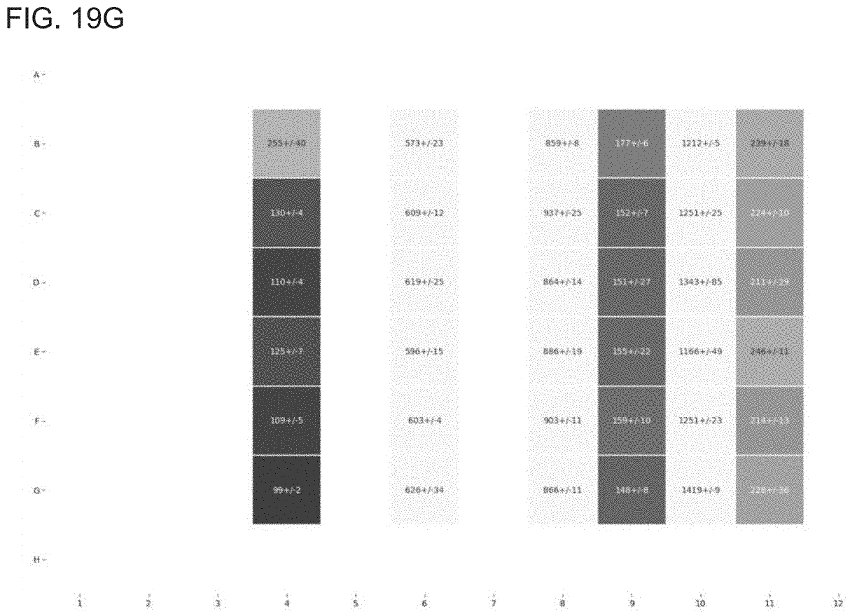

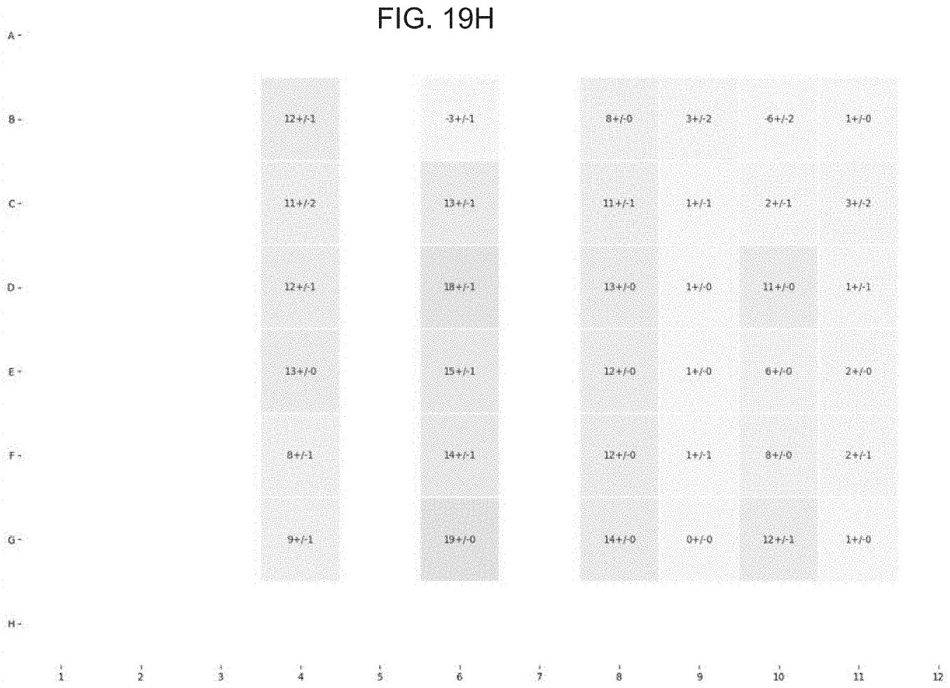

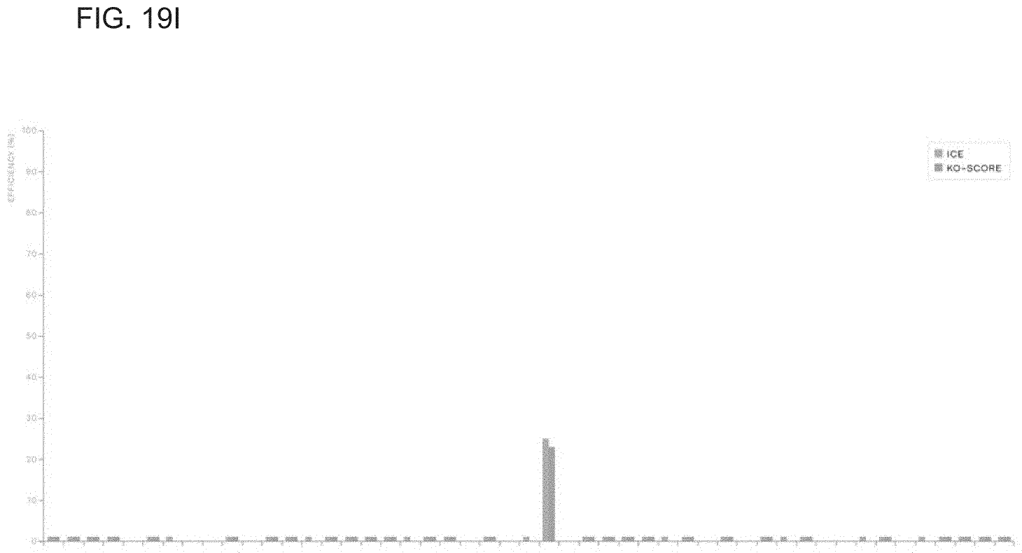

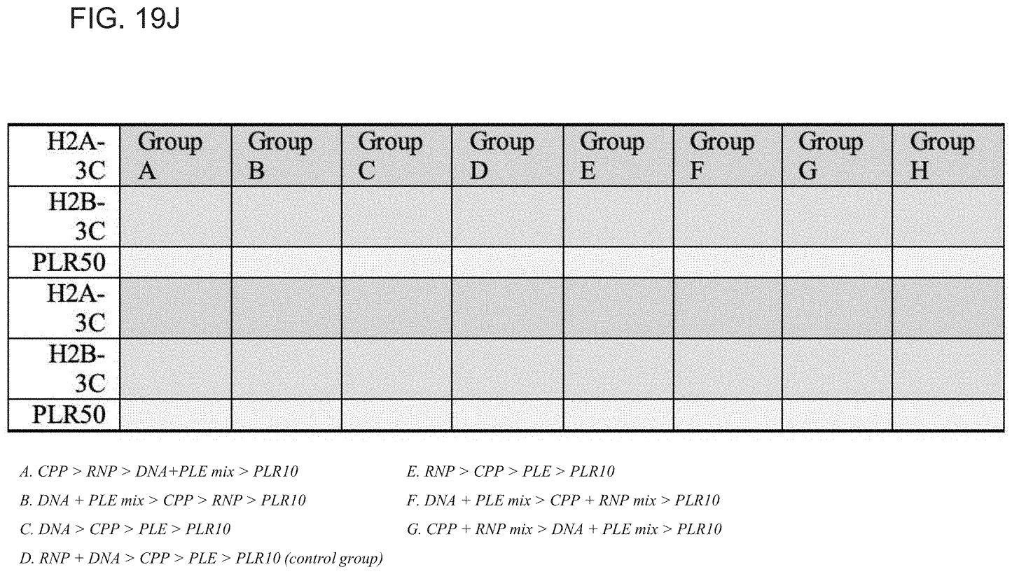

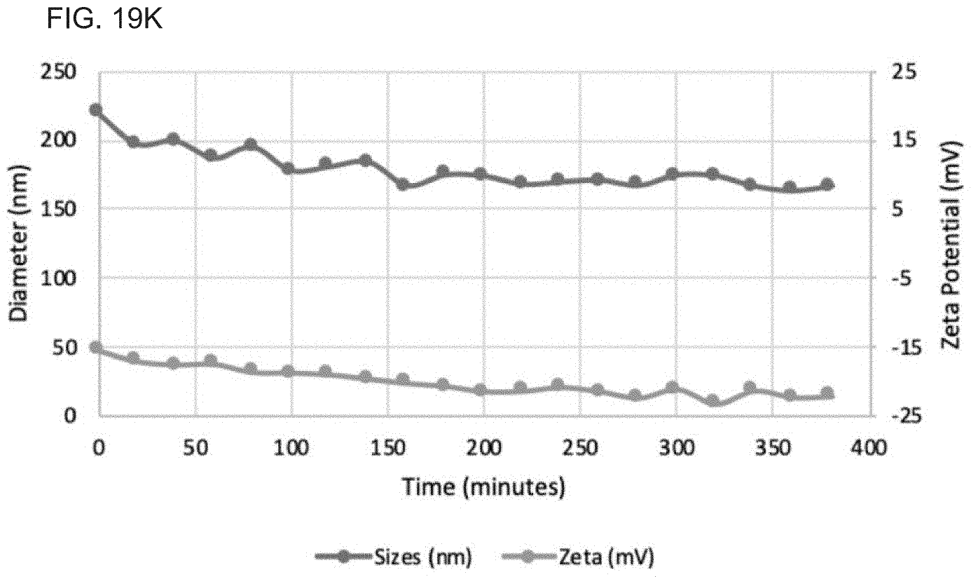

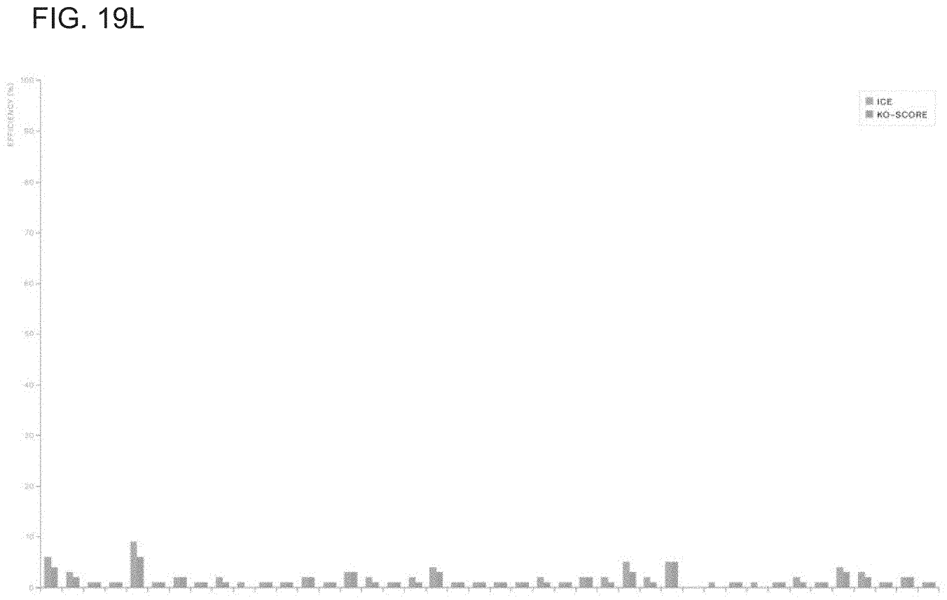

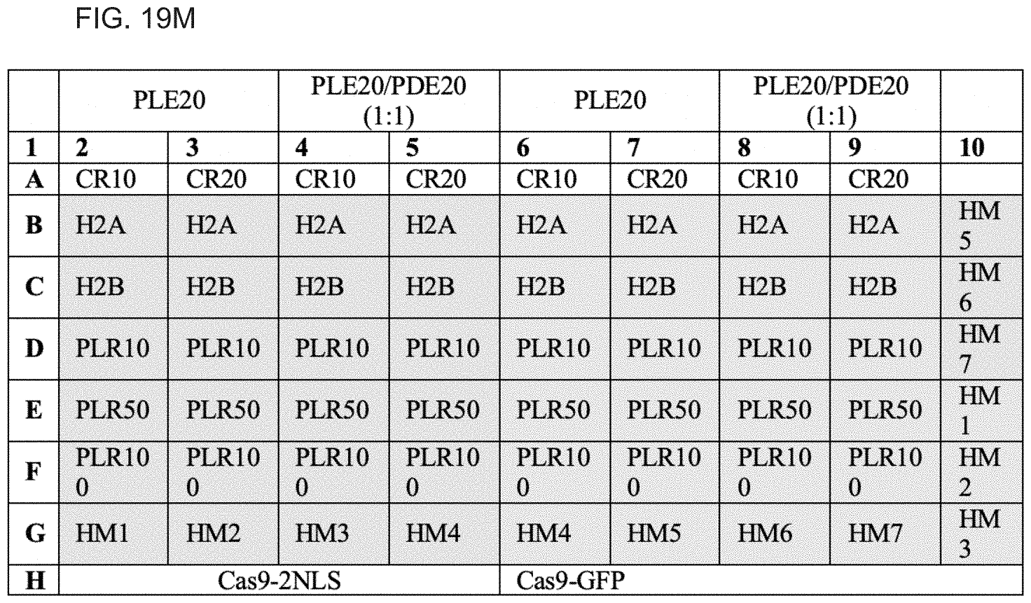

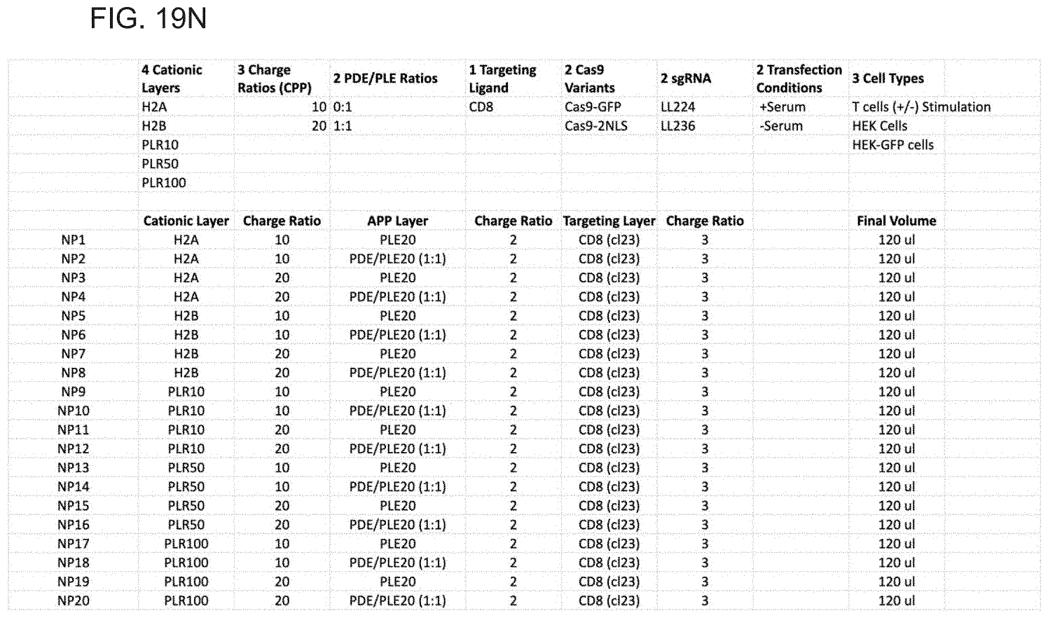

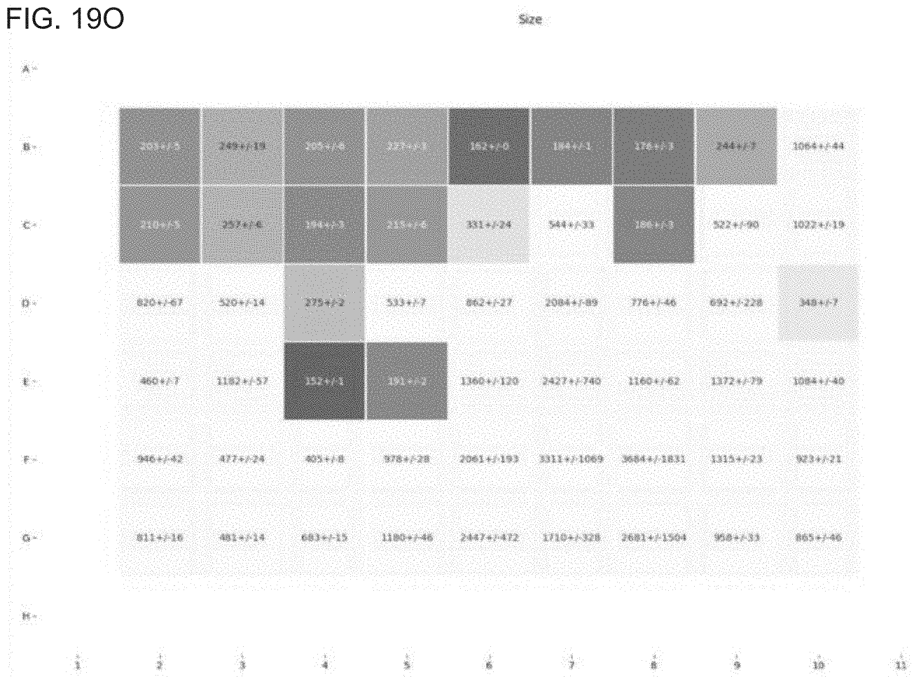

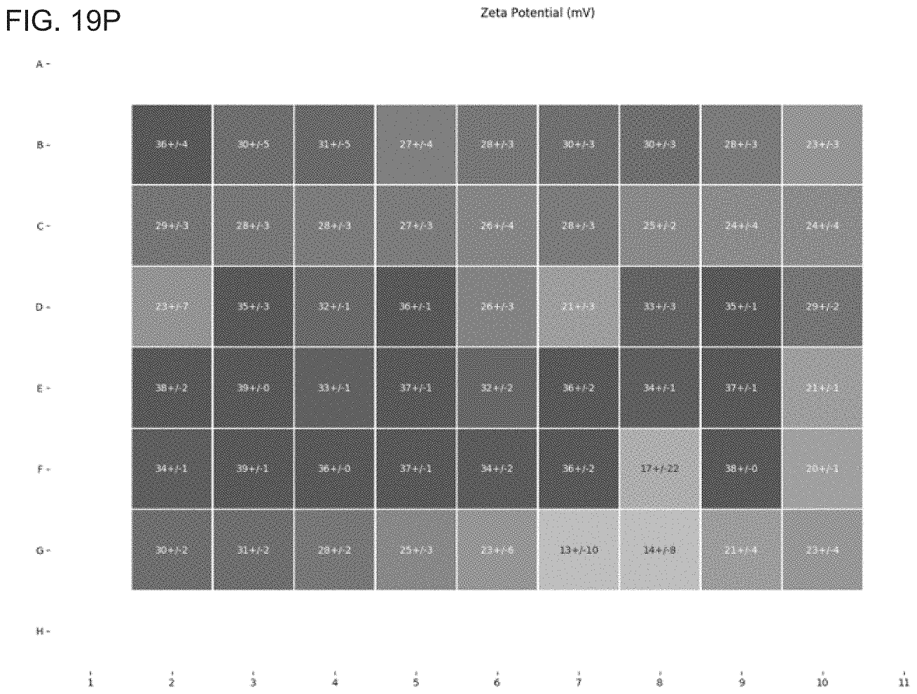

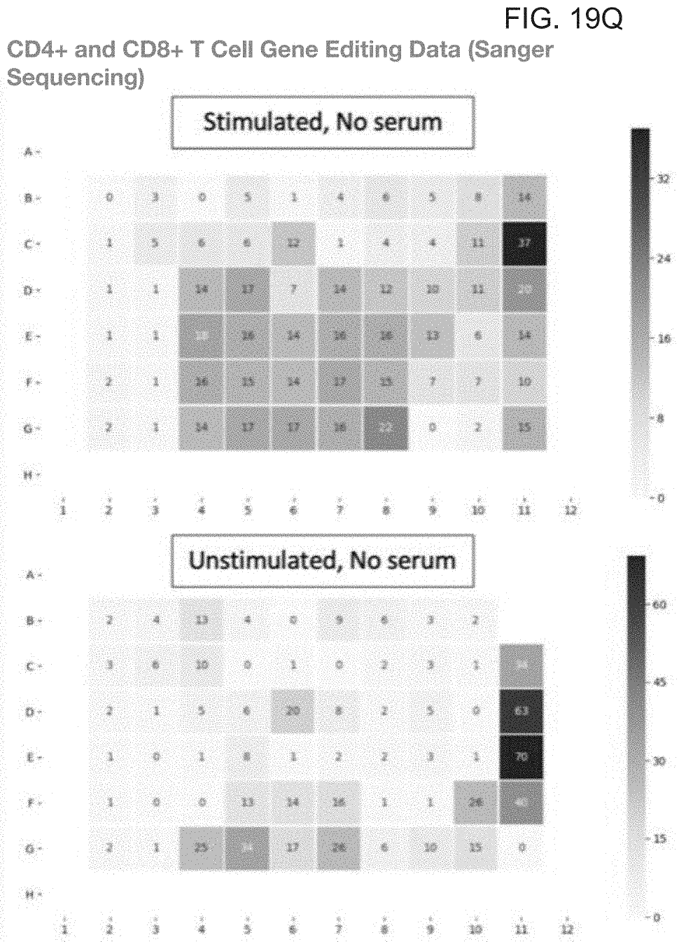

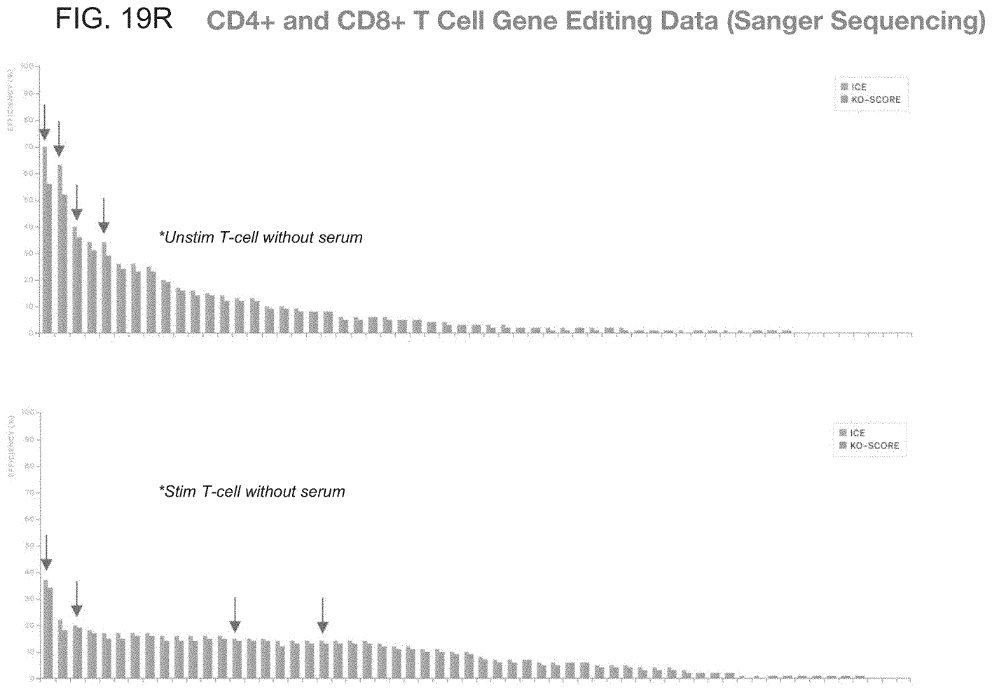

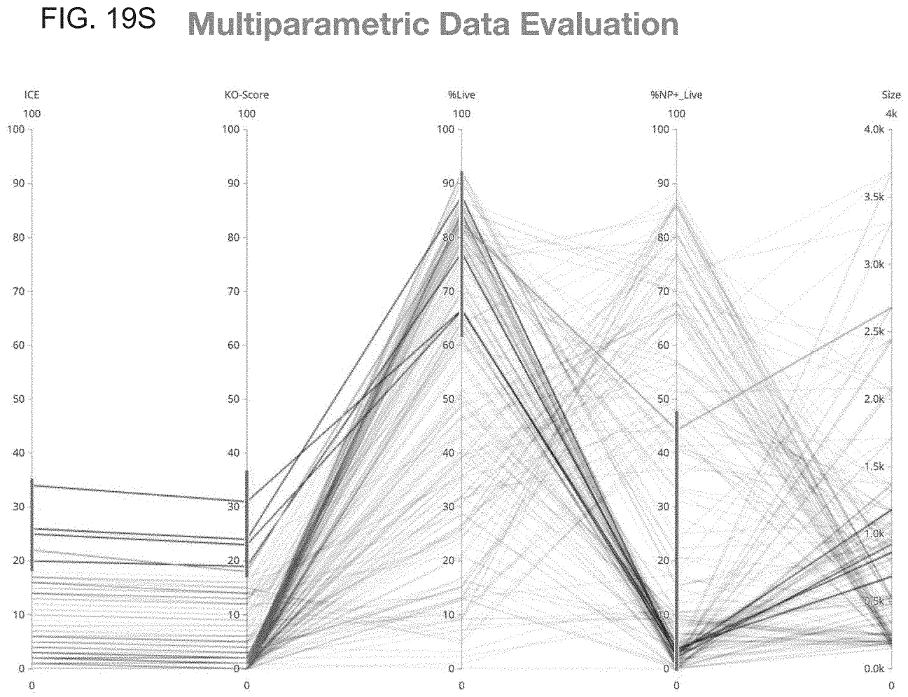

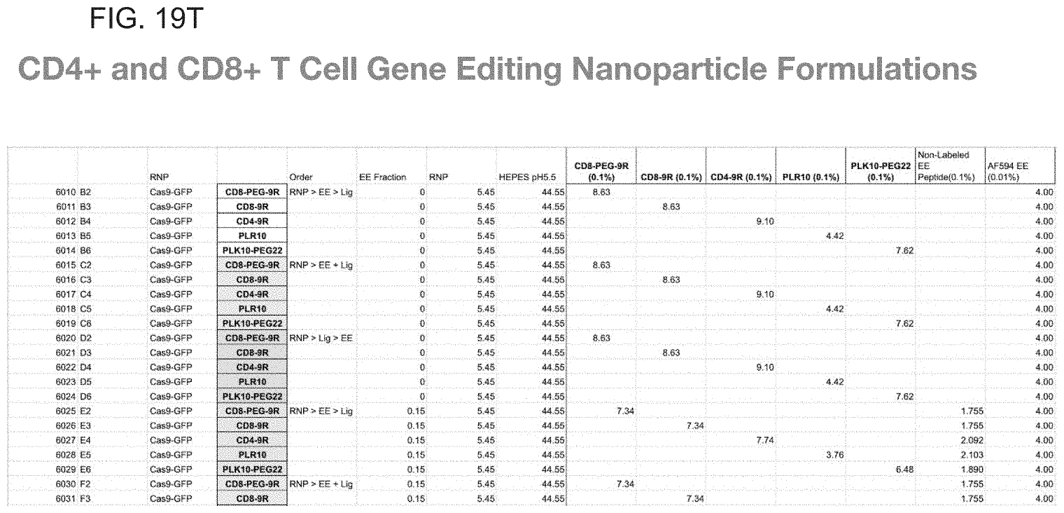

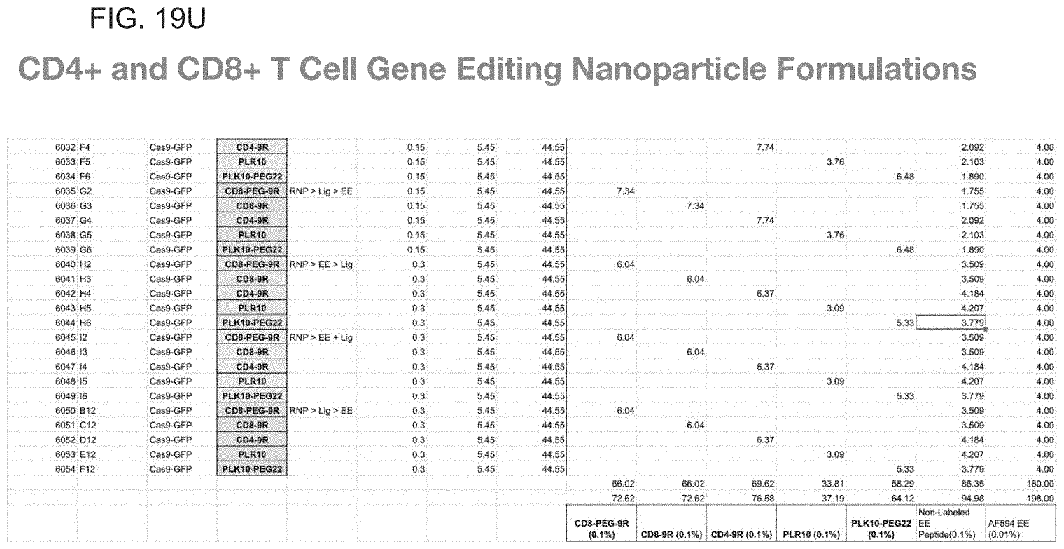

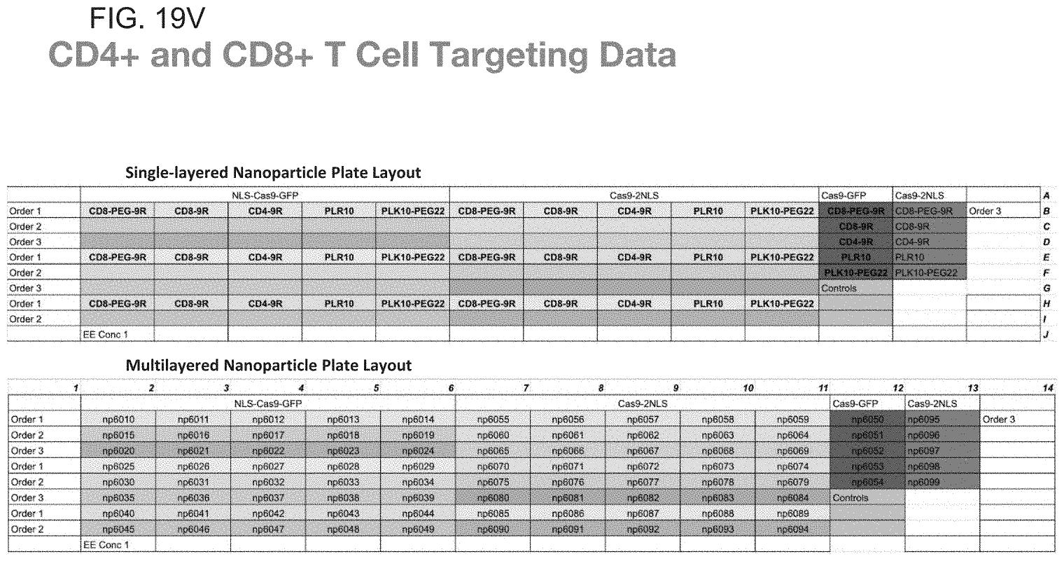

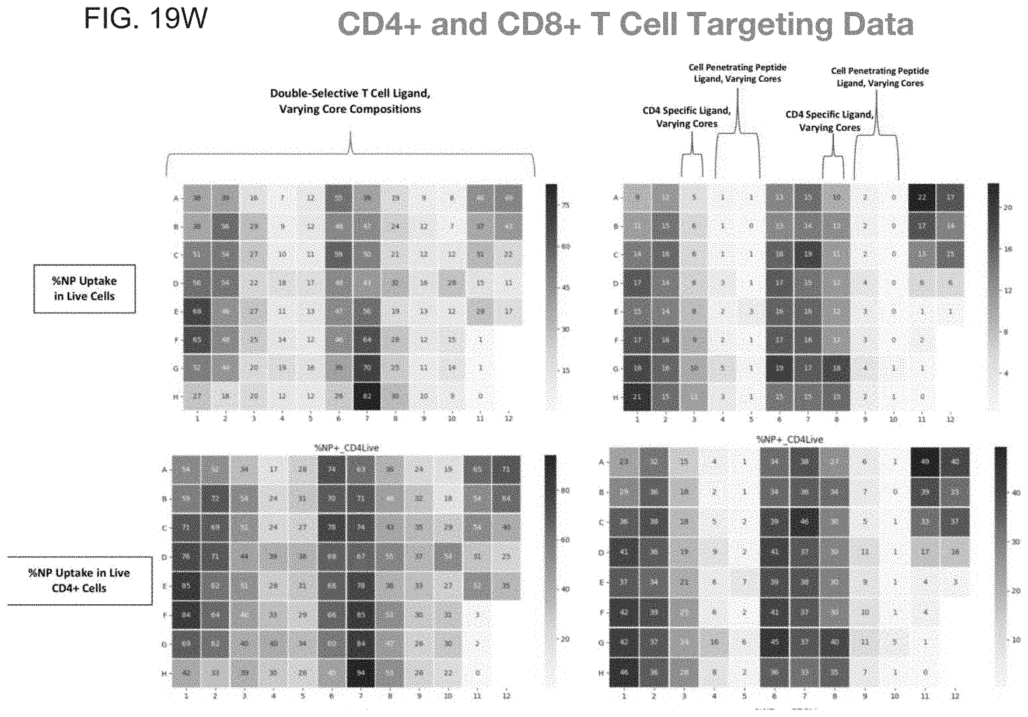

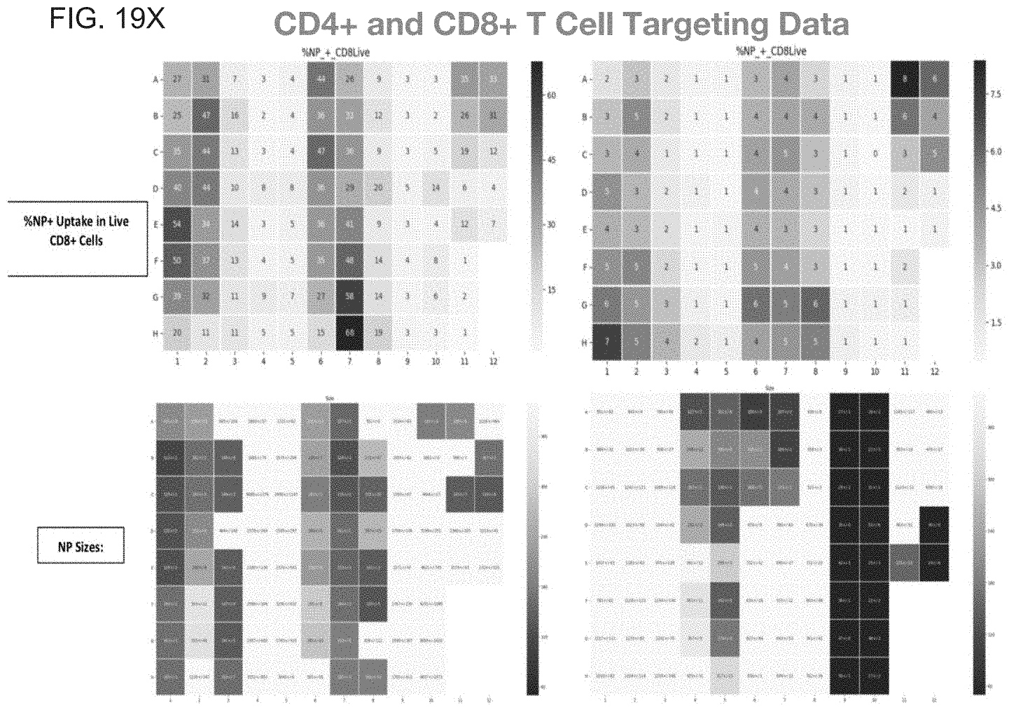

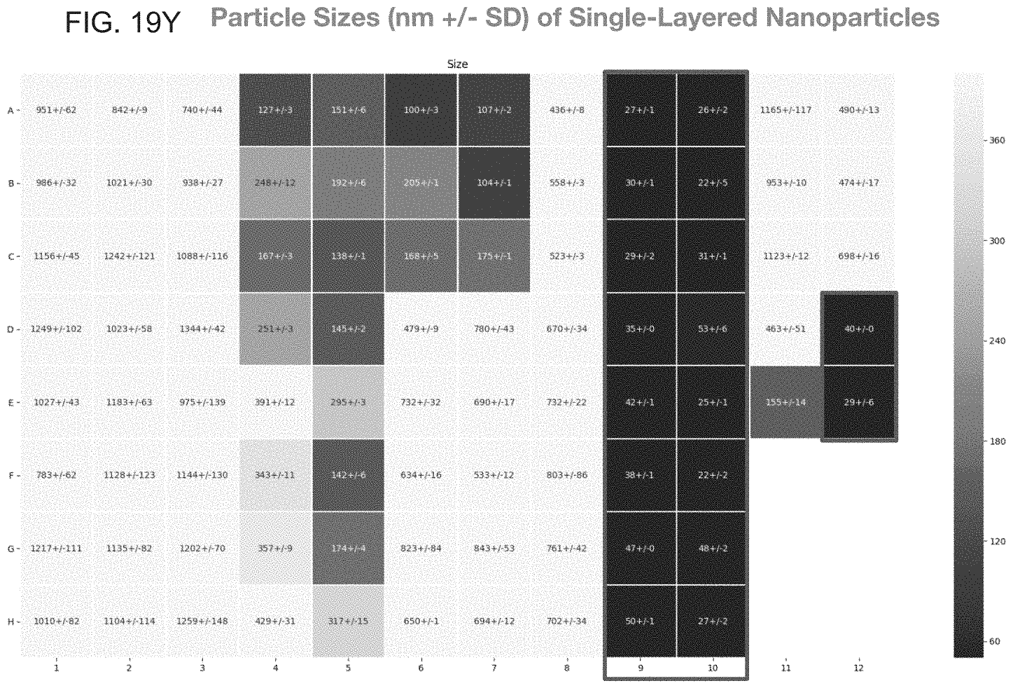

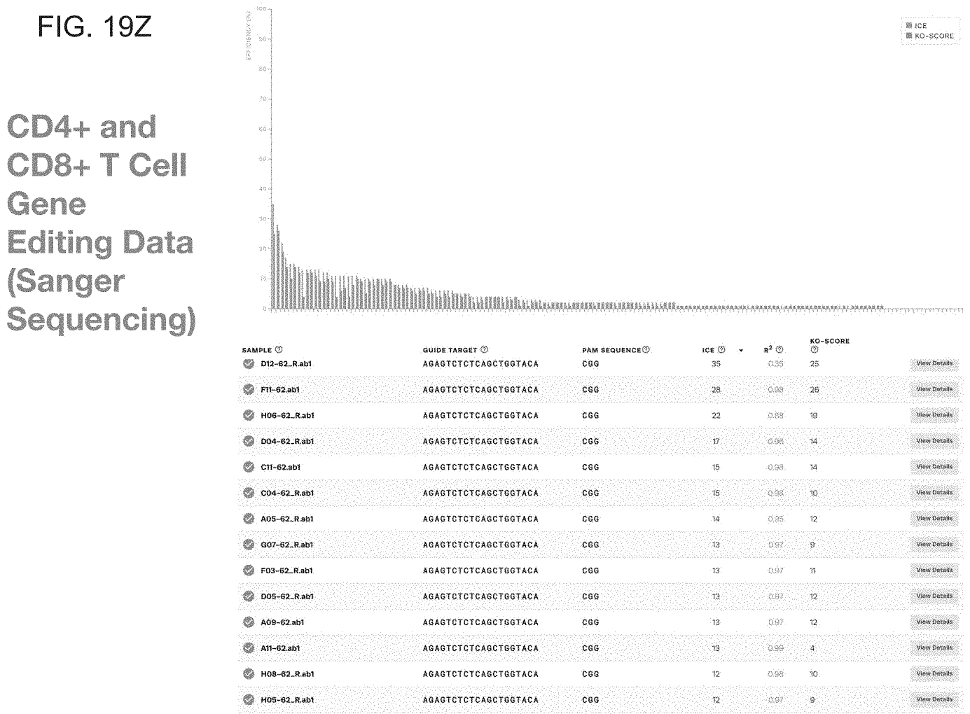

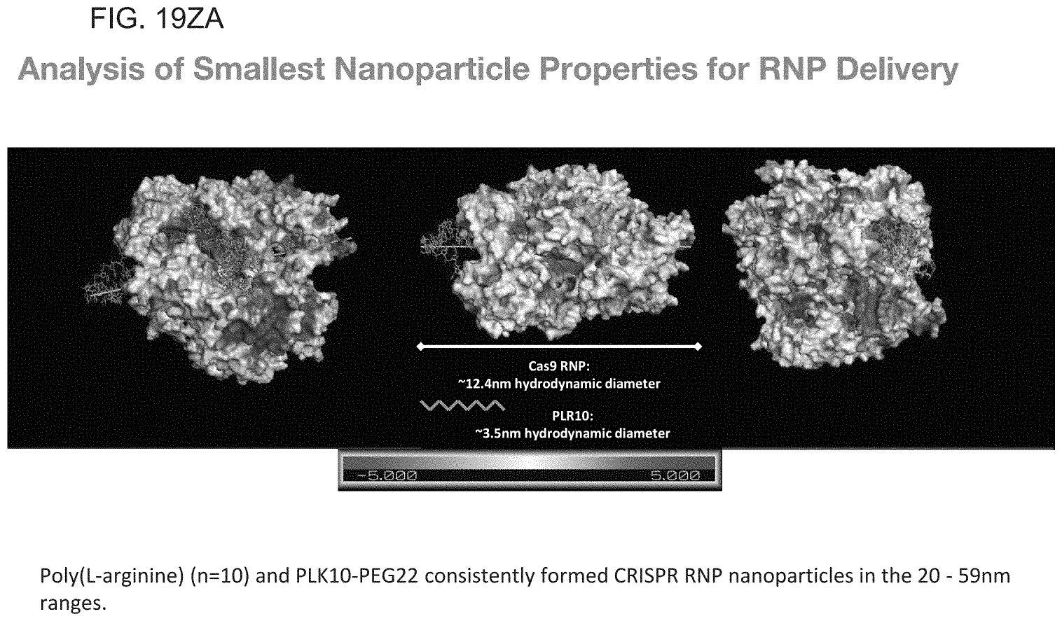

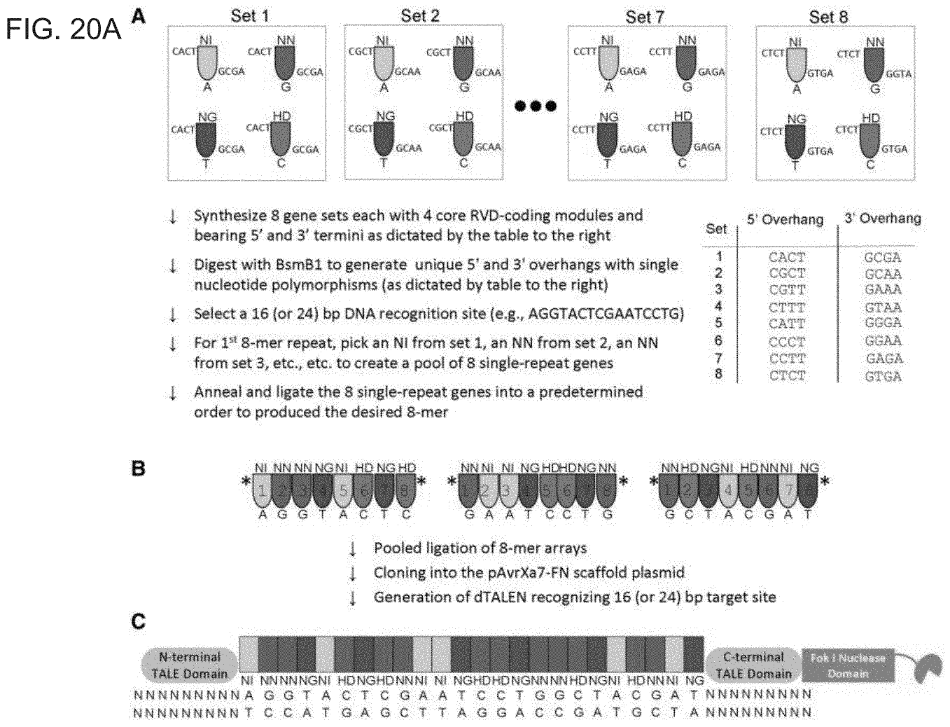

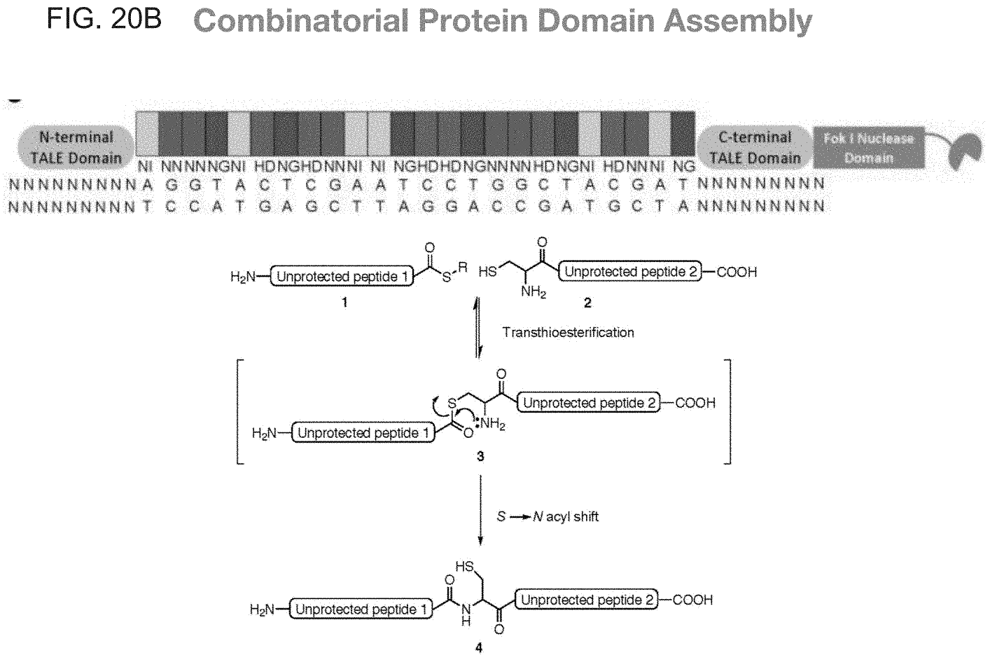

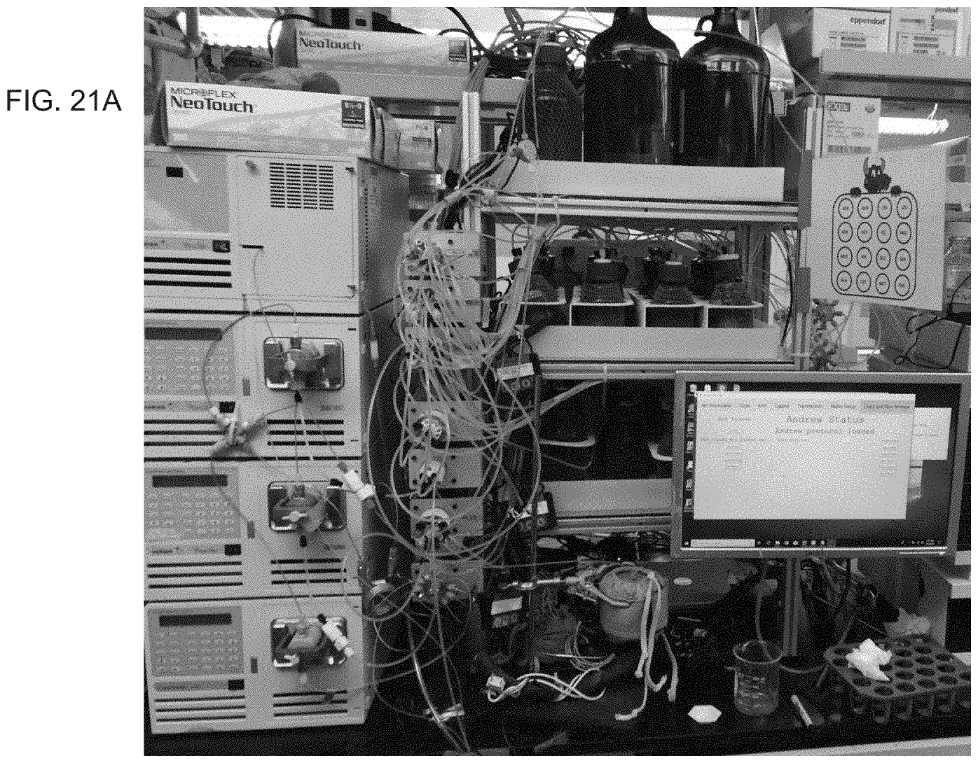

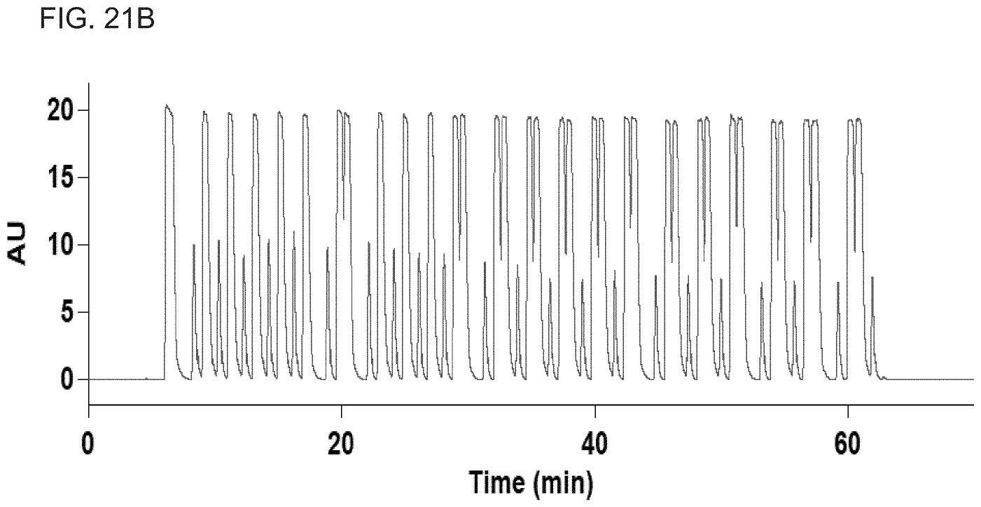

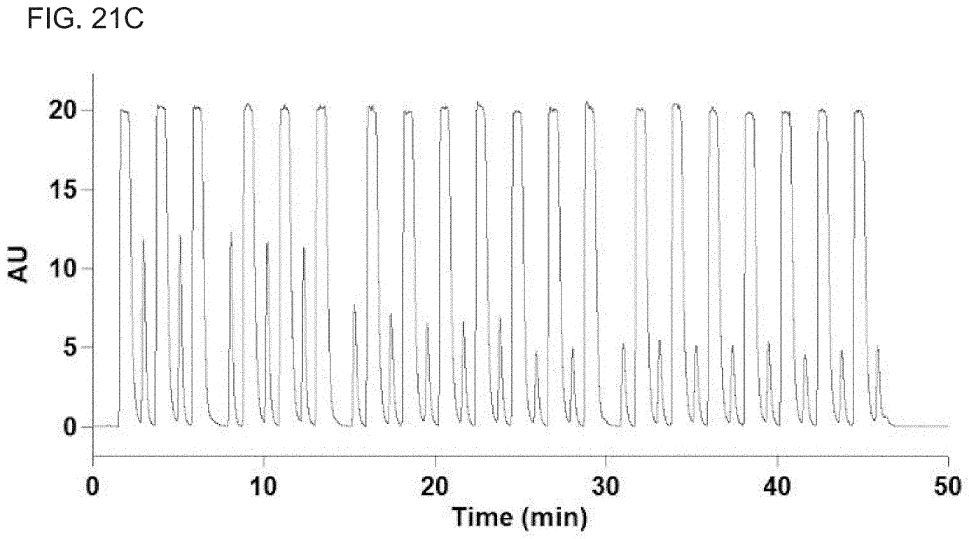

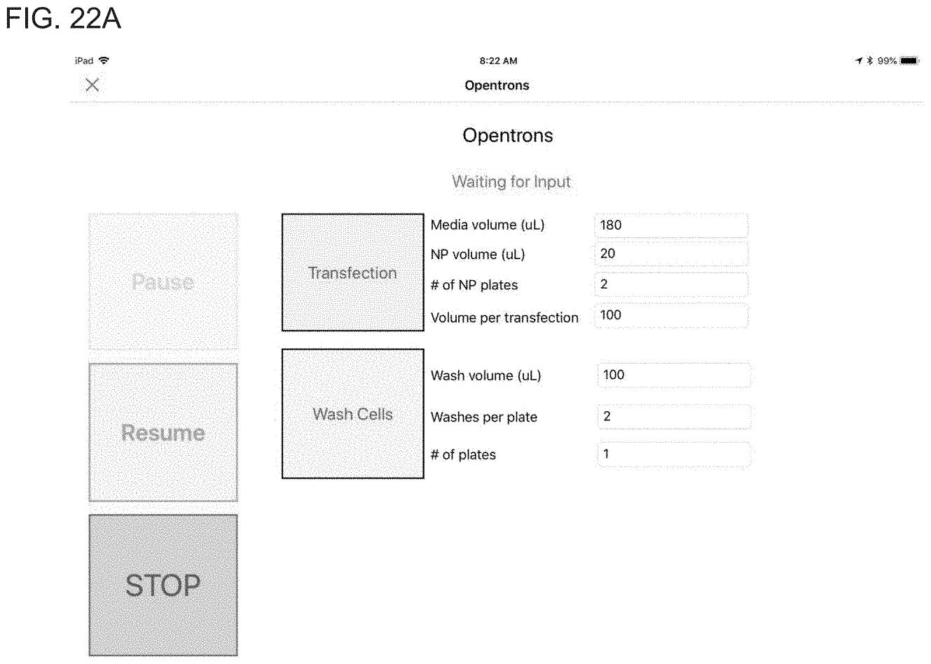

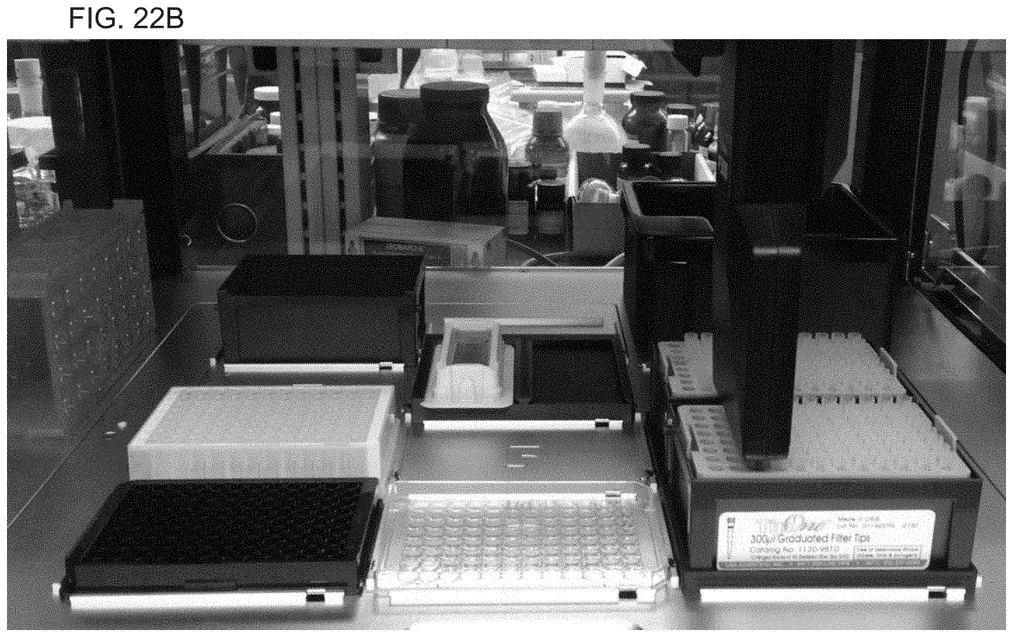

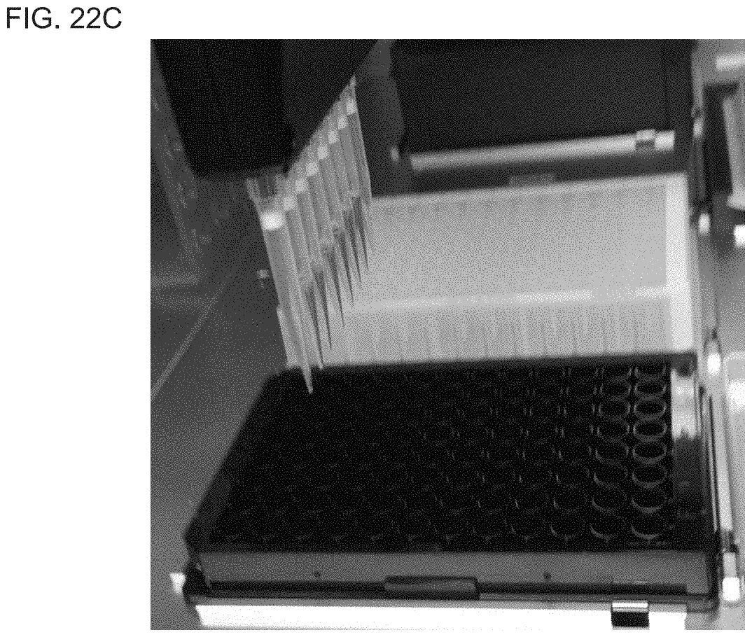

FIG. 16J1 depicts core nanoparticle+CD3epsilon-targeting ligand+CD8-targeting ligand Cas9 uptake in T cells and PBMC. Heterodivalent combination of ligands targeting CD3 and CD8.

FIG. 16J2 depicts the T cell data corresponding to FIG. 16J1.

FIG. 16J3 depicts the PBMC data corresponding to FIG. 16J1.

FIG. 16K1 depicts core nanoparticle+CD3epsilon ligand+CD80-derived CD28-targeting ligand Cas9 uptake in T cells and PBMC. Heterodivalent combination of ligands targeting CD3 and CD28 (derived from CD80).

FIG. 16K2 depicts the T cell data corresponding to FIG. 16K1.

FIG. 16K3 depicts the PBMC data corresponding to FIG. 16K1.

FIG. 16L1 depicts core nanoparticle+CD3epsilon ligand+CD86-derived CD28-targeting ligand Cas9 uptake in T cells and PBMC. Heterodivalent combination of ligands targeting CD3 and CD28 (derived from CD86).

FIG. 16L2 depicts the T cell data corresponding to FIG. 16L1.

FIG. 16L3 depicts the PBMC data corresponding to FIG. 16L1.

FIG. 16M1 depicts core nanoparticle+CD3epsilon ligand+IL2-derived IL2R-targeting ligand Cas9 uptake in T cells and PBMC. Heterodivalent combination of ligands targeting CD3 and IL2R.

FIG. 16M2 depicts the T cell data corresponding to FIG. 16M1.

FIG. 16M3 depicts the PBMC data corresponding to FIG. 16M1.

FIG. 16N1 depicts core nanoparticle+CD3epsilon ligand+PLR10 cell penetrating peptide Cas9 uptake in T cells and PBMC. Poly(L-Arginine) coating along with CD3 ligand greatly reduces efficacy from 26%.

FIG. 16N2 depicts the T cell data corresponding to FIG. 16N1.

FIG. 16N3 depicts the PBMC data corresponding to FIG. 16N1.

FIG. 1601 depicts core nanoparticle+CD80-derived CD28-targeting ligand+CD86-derived CD28-targeting ligand Cas9 uptake in T cells and PBMC. Heterodivalent combination of two CD28 ligands. Mimics antigen presenting cells: CD80+CD86 co-presentation to CD28 on T-cells. Improves transduction efficiency compared to CD80- or CD86-derived monovalent samples.

FIG. 1602 depicts the T cell data corresponding to FIG. 1601.

FIG. 1603 depicts the PBMC data corresponding to FIG. 1601.

FIG. 16P1 depicts core nanoparticle+CD3epsilon ligand+CD86-derived CD28-targeting ligand+CD8-targeting ligand Cas9 uptake in T cells and PBMC. Heterotrivalent surface targeting CD3, CD28 and CD. Slight bias of CD8+ T-cell targeting.

FIG. 16P2 depicts the T cell data corresponding to FIG. 16P1.

FIG. 16P3 depicts the PBMC data corresponding to FIG. 16P1.

FIG. 16Q1 depicts core nanoparticle+CD3epsilon ligand+CD8-targeting ligand+IL2-derived IL2R-targeting ligand Cas9 uptake in T cells and PBMC. Heterotrivalent surface targeting CD3, CD8, and IL2R. Slight bias of CD8+ T-cell targeting. ˜44.4% efficient CD8+ T Cell targeting.

FIG. 16Q2 depicts the T cell data corresponding to FIG. 16Q1.

FIG. 16Q3 depicts the PBMC data corresponding to FIG. 16Q1.

FIG. 16R1 depicts core nanoparticle+CD3epsilon ligand+CD80-derived CD28-targeting ligand+CD8-targeting ligand Cas9 uptake in T cells and PBMC. Heterotrivalent surface targeting CD3, CD28, and CD8. ˜5% bias in targeting CD8+vs. CD4+ T-cells. ˜43.9% efficient CD8+ T-cell targeting.

FIG. 16R2 depicts the T cell data corresponding to FIG. 16R1.

FIG. 16R3 depicts the PBMC data corresponding to FIG. 16R1.

FIG. 16S1 depicts core nanoparticle+CD3epsilon ligand+CD86-derived CD28-targeting ligand+CD80-derived CD28-targeting ligand Cas9 uptake in T cells and PBMC.Heterotrivalent surface targeting CD3 and CD28 (mimicking CD80 and CD86 co-presentation). Reduction in uptake vs. CD8-containing heterotrivalent surface without CD28(86). ˜4% bias in targeting CD8+vs. CD4+ T-cells.

FIG. 16S2 depicts the T cell data corresponding to FIG. 16S1.

FIG. 16S3 depicts the PBMC data corresponding to FIG. 16S1.

FIG. 16T1 depicts core nanoparticle+CD8-targeting ligand+CD80-derived CD28-targeting ligand+CD86-derived CD28-targeting ligand Cas9 uptake in T cells and PBMC. Heterotrivalent surface targeting CD8 and CD28 (mimicking CD80 and CD86 co-presentation). Efficient CD8+ T-cell targeting. ˜6% bias in targeting CD8+vs. CD4+ T-cells.

FIG. 16T2 depicts the T cell data corresponding to FIG. 16T1.

FIG. 16T3 depicts the PBMC data corresponding to FIG. 16T1.

FIG. 16U1 depicts core nanoparticle+CD8-targeting ligand+CD80-derived CD28-targeting ligand+IL2-derived IL2R-targeting ligand Cas9 uptake in T cells and PBMC. Heterotrivalent surface targeting CD8, CD28(80) and IL2R. Efficient CD8+ T-cell targeting. ˜6% bias in targeting CD8+vs. CD4+ T-cells.

FIG. 16U2 depicts the T cell data corresponding to FIG. 16U1.

FIG. 16U3 depicts the PBMC data corresponding to FIG. 16U1.

FIG. 16V1 depicts core nanoparticle+CD8-targeting ligand+CD86-derived CD28-targeting ligand+IL2-derived IL2R-targeting ligand Cas9 uptake in T cells and PBMC. Heterotrivalent surface targeting CD8, CD28(86) and IL2R. Efficient CD8+ T-cell targeting. ˜6% bias in targeting CD8+vs. CD4+ T-cells.

FIG. 16V2 depicts the T cell data corresponding to FIG. 16V1.

FIG. 16V3 depicts the PBMC data corresponding to FIG. 16V1.

FIG. 16W depicts exemplary colocalization studies performed on human primary T cells. Cells, nuclei and nanoparticles are segmented and pixel overlap coefficients are determined in order to generate real-time data of nanoparticle transfection efficiency, endosomal localization and escape, and/or nuclear uptake. In this embodiment, the “nanoparticles” channel is an EGFP-Cas9 protein.

FIG. 16X depicts exemplary colocalization coefficients (nanoparticles+ cells) as determined in human primary T cells. Cells, nuclei and nanoparticles are segmented and pixel overlap coefficients are determined in order to generate real-time data of nanoparticle transfection efficiency, endosomal localization and escape, and/or nuclear uptake. In this embodiment, the “nanoparticles” channel is an EGFP-Cas9 protein. Shown are % of cells with nanoparticles colocalized with them as determined by microscopy at each time-point. Images were acquired via a BioTek Cytation V under continuous incubation in 96-well plates and a 20× objective.

FIG. 16Y depicts exemplary colocalization coefficients (nanoparticles+ cells) as determined in human primary T cells. Cells, nuclei and nanoparticles are segmented and pixel overlap coefficients are determined in order to generate real-time data of nanoparticle transfection efficiency, endosomal localization and escape, and/or nuclear uptake. In this embodiment, the “nanoparticles” channel is an EGFP-Cas9 protein. Shown are % of cells with nanoparticles colocalized with them as determined by microscopy at each time-point. Images were acquired via a BioTek Cytation V under continuous incubation in 96-well plates and a 20× objective.

FIG. 16Z depicts exemplary colocalization coefficients (nanoparticles+nuclei) as determined in human primary T cells. Cells, nuclei and nanoparticles are segmented and pixel overlap coefficients are determined in order to generate real-time data of nanoparticle transfection efficiency, endosomal localization and escape, and/or nuclear uptake. In this embodiment, the “nanoparticles” channel is an EGFP-Cas9 protein. Shown are % of cells with nanoparticles colocalized with them as determined by microscopy at each time-point. Images were acquired via a BioTek Cytation V under continuous incubation in 96-well plates and a 20× objective.

FIG. 16ZA depicts exemplary colocalization coefficients (nanoparticles+nuclei) as determined in human primary T cells. Cells, nuclei and nanoparticles are segmented and pixel overlap coefficients are determined in order to generate real-time data of nanoparticle transfection efficiency, endosomal localization and escape, and/or nuclear uptake. In this embodiment, the “nanoparticles” channel is an EGFP-Cas9 protein. Shown are % of cells with nanoparticles colocalized with them as determined by microscopy at each time-point. Images were acquired via a BioTek Cytation V under continuous incubation in 96-well plates and a 20× objective.

FIG. 16ZB depicts super-resolution microscopy of nanoparticle-transfected human primary T cells. Shown is CRISPR Cas9-EGFP (green) in the human primary T cell (red) nucleus (blue).

FIG. 16ZC depicts super-resolution microscopy of nanoparticle-transfected human primary T cells. Shown is CRISPR Cas9-EGFP (green) in the human primary T cell (red) nucleus (blue).

FIG. 17A depicts bright field and Cy5 channel imaging of nanoparticle uptake in human CD34+ hematopoietic stem cells (left). Plate layout (right, n=6). Corresponding TEM images shown in FIGS. 17B—171. Corresponding flow cytometry data shown in FIGS. 17J-17S.

FIG. 17B depicts TEM micrographs of Cy5 mRNA+PLR10+PLE20 nanoparticles. Left scale bar=200 nm. Right scale bar=50 nm.

FIG. 17C depicts a TEM micrograph of Cy5 mRNA+PLR50+PLE20 nanoparticles.

FIG. 17D depicts TEM micrographs of Cy5 mRNA+E-selectin ligand+PLE20 nanoparticles.

FIG. 17E depicts TEM micrographs of Cy5 mRNA+equimolar anchor charge contributions between E-selectin ligand vs. c-kit ligand (SCF fragment)+PLE20 nanoparticles.

FIG. 17F depicts TEM micrographs of Cy5 mRNA+c-kit ligand (SCF fragment)+PLE20 nanoparticles.

FIG. 17G depicts TEM micrographs of Cy5 mRNA+PLK10-PEG22+PLE20 nanoparticles.

FIG. 17H depicts TEM micrographs of Cy5 mRNA+Lipofectamine MessengerMAX (0.75 uL Lipofectamine MessengerMAX reagent per 1 ug mRNA).

FIG. 17I depicts TEM micrographs of Cy5 mRNA+Lipofectamine MessengerMAX (1.5 uL Lipofectamine MessengerMAX reagent per 1 ug mRNA).

FIG. 17J depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+PLR10+PLE20 nanoparticles.

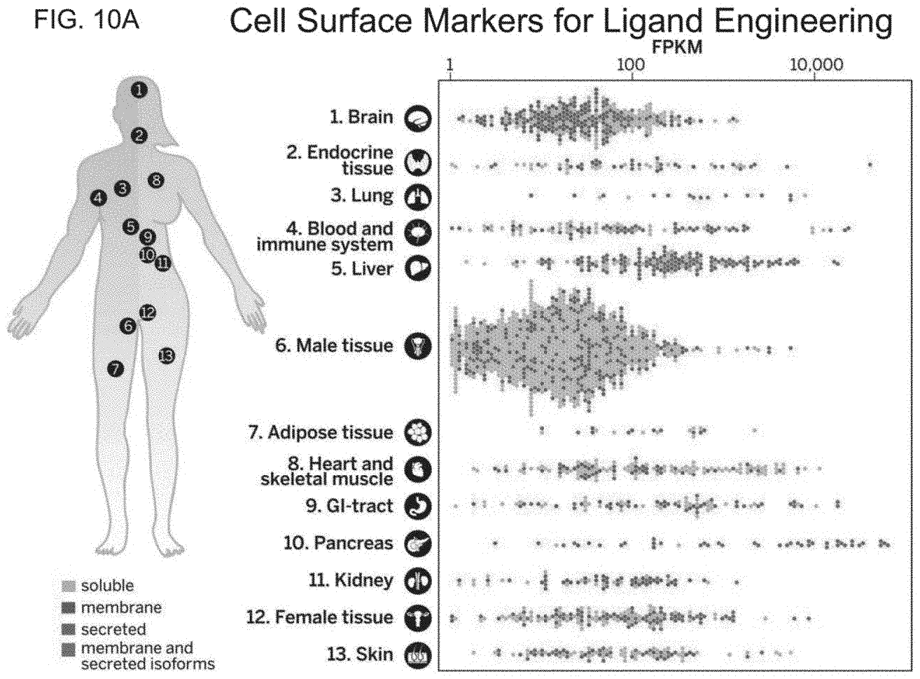

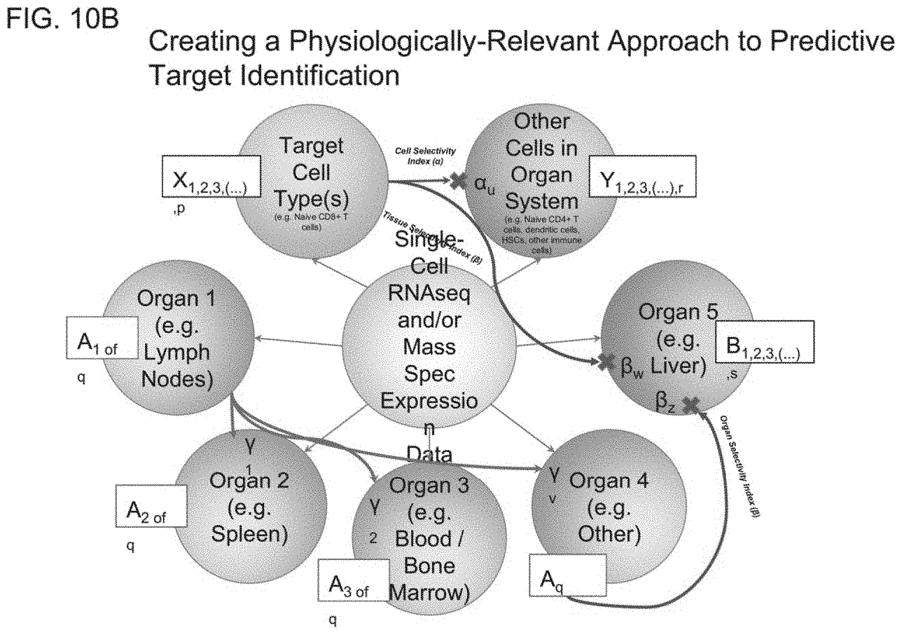

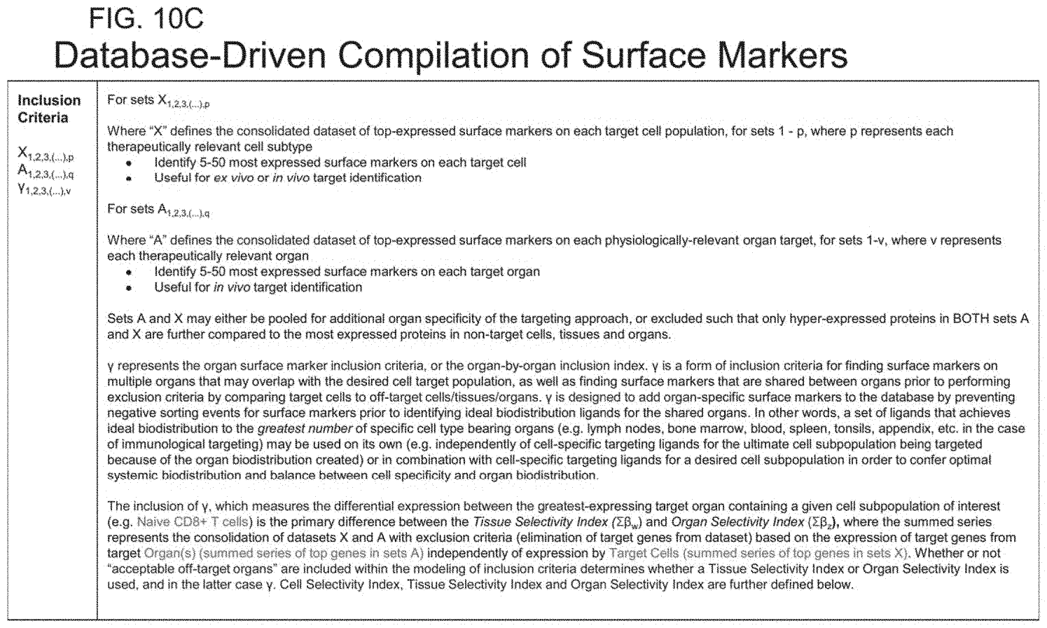

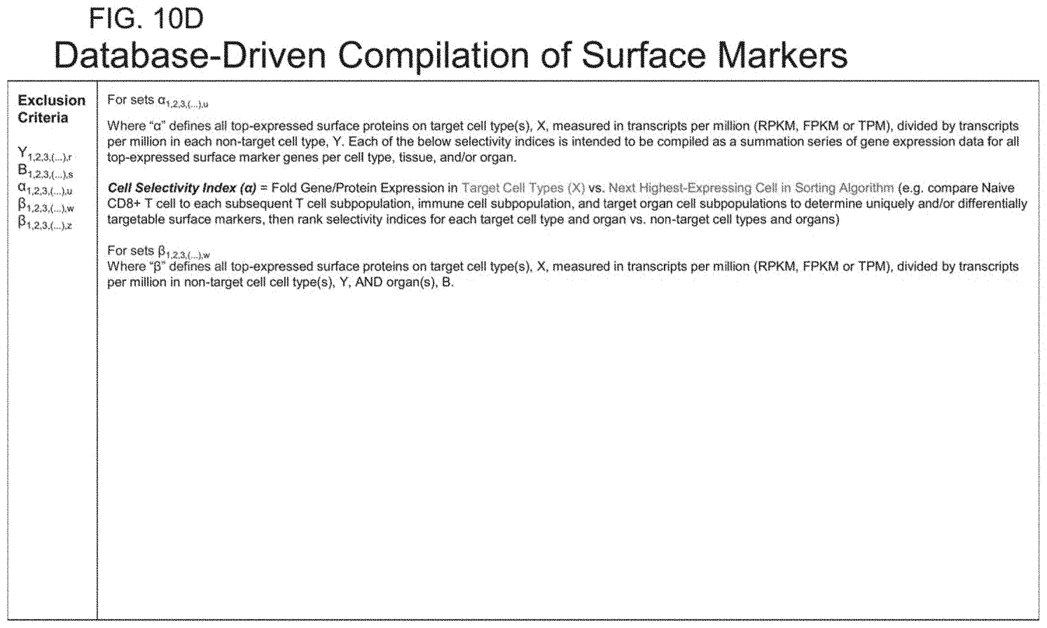

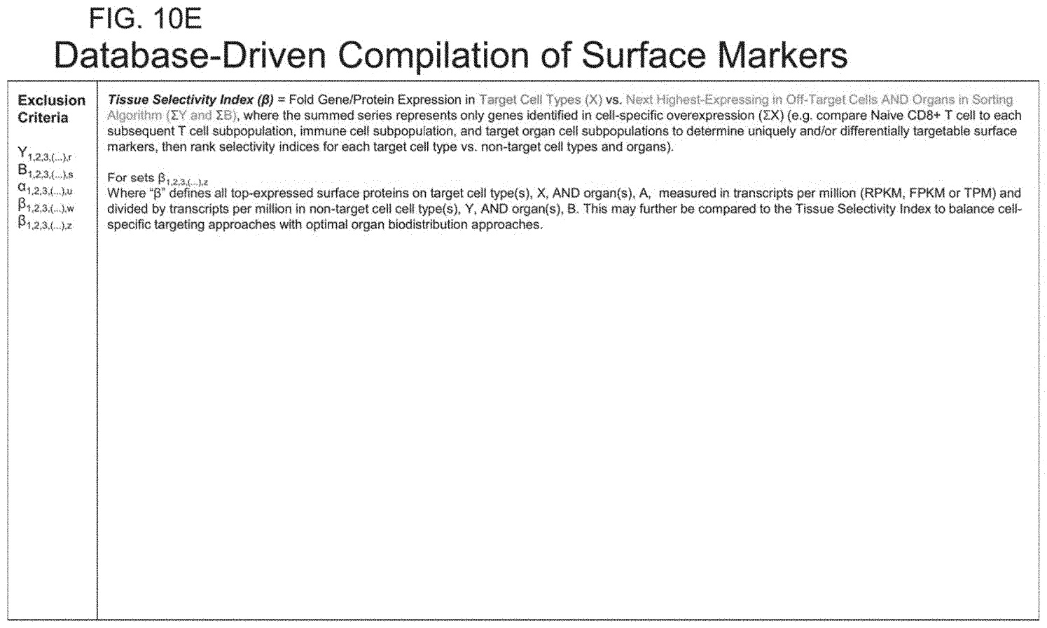

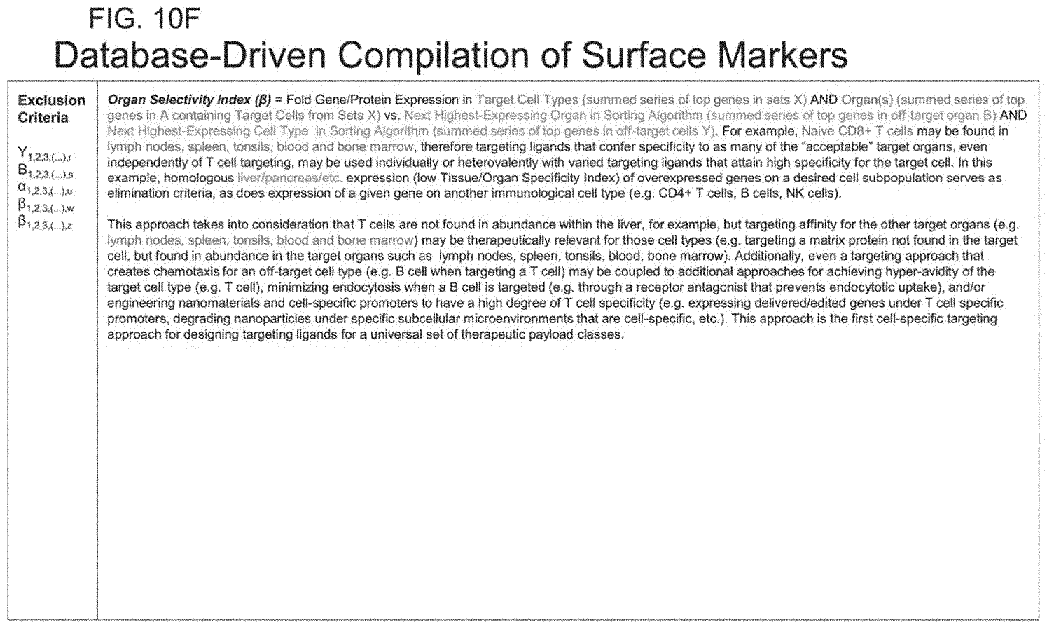

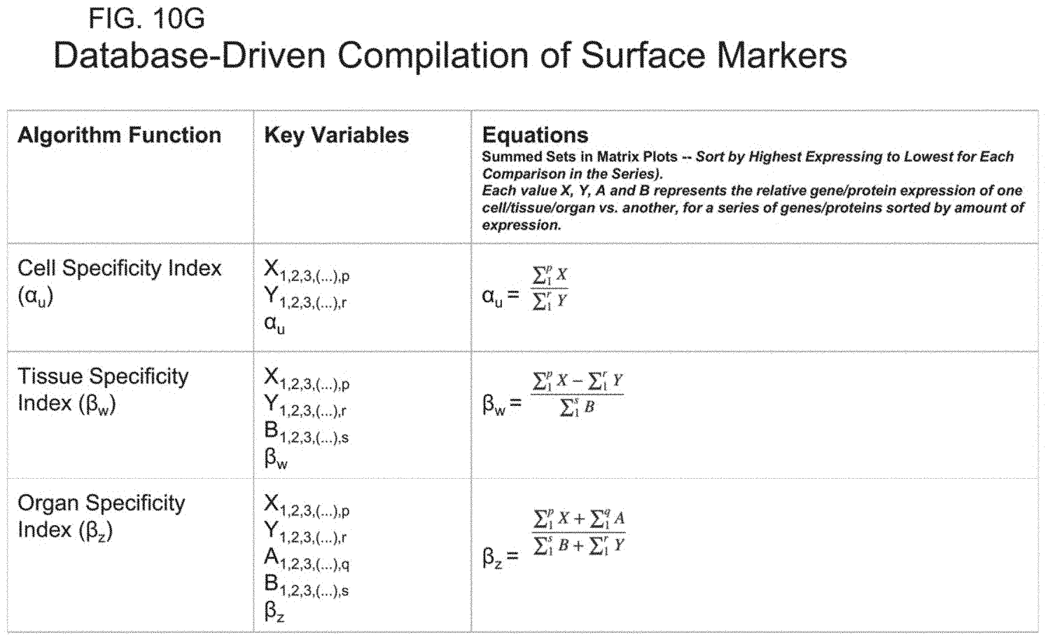

FIG. 17K depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+PLR50+PLE20 nanoparticles. This formulation outperforms both Lipofectamine MessengerMAX groups (FIGS. 10P and 10Q) in terms of CD34+live non-apoptotic cell transfection efficiency.

FIG. 17L depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+E-selectin ligand+PLE20 nanoparticles.

FIG. 17M depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+equimolar anchor charge contributions between E-selectin ligand AND c-kit ligand (SCF fragment)+PLE20 nanoparticles.

FIG. 17N depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+c-kit ligand (SCF fragment)+PLE20 nanoparticles.

FIG. 17O depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+PLK10-PEG22+PLE20 nanoparticles.

FIG. 17P depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+Lipofectamine MessengerMAX (0.75 uL Lipofectamine MessengerMAX reagent per 1 ug mRNA).

FIG. 17Q depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. This formulation corresponds to Cy5 mRNA+Lipofectamine MessengerMAX (1.5 uL Lipofectamine MessengerMAX reagent per 1 ug mRNA).

FIG. 17R depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. Shown is a non-transfected control (NTC).

FIG. 17S depicts flow cytometry data of Cy5 mRNA transfections in CD34+HSCs. Cells were cultured and Cy5 EGFP mRNA (998 nt, TriLink) and cellular uptake was assessed 1 d post-transfection via an Attune NxT flow cytometer. Stains were performed for Caspase-3,7, ZombieNearIR, and CD34 and Cy5+ cells were explored for viability and transfection efficiency. Shown is a negative bead control (NBC).

FIG. 18A depicts a multifunctional peptide sequence, with image of a bioresponsive functional domain (in this case an endosomolytic domain). The FDIIKKIAES domain of this particular peptide may have additional utility as an endosomolytic/helical/spacer domain, with an optional cleavage domain (e.g. FKFL or protease cleavage site), and a subsequent display of an optional ligand for cellular receptor affinity (PDB ID 1VM5).

FIG. 18B depicts the first 62 amino acids of statherin, whereby either the signal peptide sequence MKFLVFAFILALMVSMIGA or a longer sequence containing DSepSepEEKFLRRIGRFG (Sep=phosphoserine) may be used to confer enhanced lung “secretomimetic” behavior of nanoparticles. In addition to targeting ligands being utilized that correspond to surface markers on a target cell type, secreted proteins may also be used to enhance nanoparticle properties in a specific microenvironment. This protein is upregulated 1719× in the lung cancer marker dataset that we examined as an organ-selective marker.

FIG. 18C depicts Surfactant Protein B (see Nicholas Rego and David Koes 3Dmol.js: molecular visualization with WebGL Bioinformatics (2015) 31 (8): 1322-1324 doi:10.1093/bioinformatics/btu829). Its sequence corresponds to CWLCRALIKRIQAMIPKGGRMLPQLVCRLVLRCS and this protein is found upregulated in lung cancer as a marker with an organ specificity index of 912. This protein is upregulated 912× in the lung cancer marker dataset that we examined as an organ-selective marker. In addition to its amphipathic properties and dual terminal helical domains and “flexible” central domain, it may serve as a surface coating upon a nanoparticle through many of the “linker” and functional domain embodiments detailed elsewhere. The properties of this peptide may assist in forming protein-bound nanoparticles with pulmonary mucous-adsorptive characteristics.

FIG. 18D depicts a crystal structure of Calcitonin related polypeptide alpha (PDB ID 2JXZ.A). This protein is upregulated 78× in the lung cancer marker dataset that we examined as an organ-selective marker.

FIG. 18E depicts a structural homologue of BPI fold containing family B member 2: BPI fold containing family B member 1 (PDB ID 4KJH). Due to the sequence similarity, and despite the absence of a crystal structure for BPI fold containing family B member 2, it is possible to predict ideal sequences for extracting ligand-receptor or secreted protein-environment (secretomimetic) interactions. This protein is upregulated 23× in the lung cancer marker dataset that we examined as an organ-selective marker.

FIG. 18F depicts lung adenocarcinoma and renal cell carcinoma relative expression of Napsin A aspartic peptidase (Mol Cell Proteomics. 2014 February; 13(2):397-406. doi: 10.1074/mcp.M113.035600. Epub 2013 Dec. 5.). Napsin A aspartic peptidase interacts proteolytically with Napsin-A, which presents Napsin-A as an ideal nanoparticle constituent for Napsin A aspartic peptidase processing in lung and kidney cancers overexpressing this protease. Either the signal peptide (1-24), entire chain (1-104), or specific sequences that are cleaved as determined by mass spectroscopy of Napsin-A in the presence of Napsin A aspartic peptidase may be utilized. Similarly, Napsin A aspartic peptidase overexpression may be used along with surfactant protein B surface coatings on nanoparticles due to Napsin A aspartic peptidase's proteolytic effect on Surfactant protein B. This protein is upregulated 14× in the lung cancer marker dataset that we examined as an organ-selective marker.

FIG. 18G depicts crystal structures of a potential binding partner (top, COPS2: PDB IDs 4D10, 4D18, 4WSN) to nuclear receptor subfamily 0 group B member 1 (bottom, PDB ID 4RWV) for programming subcellular-specific behavior of a nuclear receptor (Nuclear receptor subfamily 0 group B member 1) that is overexpressed on the target cell/tissue/organ.

FIG. 18H depicts how paroxonase 3 (left, PDB ID 1v04) overexpression may be used to engineer polymer chains (right) modified with cleavable N-acyl homoserine lactone motifs in order to encourage substrate specificity through degradation in a tissue-enriched way. Various other substrates with specific cleavage activity may be used.

FIG. 18I depicts structural homologues of Keratin, type I cuticular Hal. Left: keratin 5 and 14 (PDB ID 3tnu). Top right: keratin type I cytoskeletal 14 (PDB ID 3TNU.A). Bottom right: keratin type II cytoskeletal 5 (PDB ID 3TNU.B). Keratin fragments may serve as structural homologues for cell-ECM (extracellular matrix) mimetic nanoparticle surface chemistries with specific activity in a given microenvironment (such as a tumor microenvironment, or other cell/tissue/organ). These fragments may serve as biomimetic alpha helices for nanoparticle surface stabilization, as well as for complementary binding to intermediate filaments in a tissue-enriched way. Keratin sequences natively contain many cysteine residues, and may assist in nanoparticle cross-linking following electrostatic assembly of keratin-containing sequences or functionalization of a nanoparticle surface with keratin-containing domains (e.g. alpha helices).

FIG. 18J1 depicts high homology of coils 1A, 1B, and 2 between keratin, type I cuticular Hal (top) and keratin, type I cytoskeletal 14 (bottom).

FIG. 18J2 depicts an enlarged version of the top diagram of FIG. 18J1.

FIG. 18J3 depicts an enlarged version of the bottom diagram of FIG. 18J1.

FIG. 18K1 depicts human SCF in complex with an extracellular domain of Kit (green) vs. mouse SCF (blue) prior to sequence alignment.

FIG. 18K2 depicts an enlarged version of a section of FIG. 18K1.

FIG. 18L1 depicts human SCF in complex with an extracellular domain of Kit (green) vs. mouse SCF (blue) following sequence alignment. The c-Kit receptor and SCF have high sequence homology between species, allowing higher translatability of murine to human experiments when performing SCF studies targeting ltHSC, stHSC, and/or CD34+ hematopoietic stem cells. Both mouse and human variants exhibit identical lengths for the signal peptide vs. Kit ligand domains, and high degrees of sequence alignment.

FIG. 18L2 depicts an enlarged version of a section of FIG. 18L1.

FIG. 18M depicts EMBOSS Needle sequence alignment scripting comparing human SCF (https://www.uniprot.org/uniprot/P21583) and mouse SCF isoform 1 (https://www.uniprot.org/uniprot/P20826) sequence alignments. The two proteins have 89.7% sequence similarity and share 82.8% sequence identity. Therefore, domains from each of these proteins may be used to target mouse vs. human c-Kit. Additionally, the proteins exhibit nearly identical alignment of crystal structures (FIG. 18O) despite only 82.8% sequence identity.

FIG. 18N depicts a crystal structure of the hyaluronan binding domain of human CD44 (PDB ID 1UUH) and a corresponding structure of hyaluronan/hyaluronic acid, which can readily be included upon nanoparticle surfaces or as an anionic core nanoparticle component, and may serve as a CD44-specific targeting ligand.

FIG. 18O depicts the region of CD166(28-120) which mediates CD6 binding via its N-terminal Ig-like V Type 1 domain. A signaling peptide sequence (1-17, 1-25, or 1-28) may also be utilized individually or as part of the Ig-like domain.

FIG. 18P depicts how CD166(28-120) mediates CD6 (T-cell differentiation antigen CD6) binding via its N-terminal Ig-like V Type 1 domain (square highlighted on left). The membrane-proximal CD6 SRCR domain (labeled Sc) mediates binding to the N-terminal Ig-like V Type 1 domain of CD166 (middle, PMID: 26146185). A small domain signature is identified on the C-terminus of human CD6, whereby amino acids D291-N353 (62AA) dictate binding to CD166 (top right, PMID: 26146185). Correspondingly, a small domain signature is identified on the N-terminus of human CD166, whereby amino acids F53-E118 (65AA) dictate binding to CD6. Notably, binding domains have t-shaped domains (“oppositely charged t-complementary domain”/“staple domain”) of identical size (right) and overlapping scale. Conversely, CD166 fragments may be used to target CD6, which is a T cell marker and signals for T cell activation upon binding to CD166 (typically expressed on endothelial cells). The use of this ligand and its concomitant receptor is not only restricted to lung cancer, but may also be utilized for targeting various endothelial cell and immune cell populations as part of a nanoparticle coating bearing one or more targeting ligands.

FIG. 18Q depicts two techniques for forming de novo CD6-specific ligands, whereby a triple-domain electrostatic affinity sequence matches dimensions of the binding pocket of CD6. Dimensional reduction techniques of a 2-dimensional electrostatic pocket allow for creation of short peptide sequences with corresponding electrostatic affinity for the t-shaped domain.

FIG. 18R depicts ScFv critical sequences for CD133 (prominin-1) binding.

FIG. 18S depicts hydrogen bonding residues involved in PIP binding to a1, a2 and a3 domains of Zinc-alpha-2-glycoprotein (ZAG) (PDB ID 3es6). Prolactin-induced protein interacts with Zinc-alpha-2-glycoprotein (ZAG) (PDB ID 3es6) via E229-G238 in the a3 domain, and D23, D45 and Q28 (which are less than 5AA apart if a charge-based triangulation approach for de novo ligand domains is utilized (as in FIG. 18Q). The interactions between D23, Q28 and D45 on the a1 domain of ZAG with T79, S47 and R72 on PIP can be reproduced by creating cyclical peptide sequences displaying the appropriate amino acids (D, D, Q) at the with sufficient spacing to allow for reproduction of native hydrogen bonding. Larger sequences (e.g. D23-D45 for a1 domain) may also be utilized. Correspondingly, E229-G238 from the a3 domain (a mere 10 amino acids) can be used to confer binding to G52, T59, T60 and K68 on PIP. Additional cysteine or selenocysteine substitutions at glycine residues with SH/SeH protection groups may be used to allow for initial “ring-forming” C- and N-terminal cysteine cross-linking before deprotection and subsequent attachment to an anchor or anchor-linker pairing as described elsewhere. Other linker domain sequences, PEG, and the like may be utilized in place of GGS/GGGS sequences to create the appropriate spacing structures. ZAG shows a high degree of sequence homology to MHC-I, where similar modeling approaches may be applied.

FIG. 19A depicts various buffers and pH conditions that may be utilized for achieving efficient electrostatic nanoparticle condensation (left), and associated intensity profiles of Cas9 RNPs in the 1-20 nm range (right) prior to nanoparticle formation. Prior to optimization of Cas9 “core RNP” sizes, Cas9 aggregates are formed in the ˜70-100 nm range. Optimization of buffer conditions yields acceptable RNP sizes. pH 6.5 1×PBS and 25 mM pH 6.5 HEPES yielded optimal Cas9 RNP sizes for subsequent layering of RNPs. In these embodiments, free RNP serve as “seed substrates” for subsequent nanoparticle formation, in contrast to RNA/DNA—cationic peptide interactions where there is no “seed substrate.” Therefore, presenting an as-small-as-possible RNP size at the time of nanoparticle formation will yield optimal nanoparticle properties (including <70 nm variants) that may be particularly well suited for caveolae-mediated and clathrin-mediated receptor-specific endocytic pathways due to endosomal vesicle sizes >70 nm preferentially accumulating in lysosomal and phagocytic pathways. Engagement of “long endosomal recycling pathways” and “short endosomal recycling pathways” may be utilized to optimize nanoparticle uptake into endosomal vesicles that may possess enhanced subcellular trafficking pathways for cytosolic and nuclear delivery of a variety of payloads, and these specific endosomal pathways are not present when nanoparticle sizes are sufficiently large. Optimization of seed substrate size is a key component of finding optimal nanoparticle formulations for cell-specific cellular transfection.

FIG. 19B depicts computer-assisted formulation design, whereby various ratios of poly(L-glutamic acid) and poly(D-glutamic acid) (PLE20 and PDE20) are evaluated and the associated physicochemical properties of single-layered nanoparticles (payload+outer layer) and multi-layered (payload+layer 1+layer 2++layer n) nanoparticles are gathered as a baseline for dsDNA and/or RNP and/or other nucleic acid nanoparticle synthesis. Shown are particles condensed with either poly(L-arginine) (PLR, n=100), or histone-derived cysteine-substituted cationic polypeptide sequence H2B-3C (CEVSSKGATICKKGFKKAVVKCA). Group B represents plasmid DNA (pDNA_mTagGFP2-N1), while Group E represents linear DNA (dsDNA_mTagGFP2-N1). Each component had a charge ratio of 3:1 and the anionic polymer components consisted of PLE20 and/or PDE20.

FIG. 19C depicts condensation of dsDNA payloads into nanoparticles as was evaluated using a SYBR Gold fluorescent assay. The table details delta in fluorescence calculated as—{(Fluorescence value for sample at time x-fluorescence value of naked plasmid or dsDNA controls at time x)/fluorescence value of naked plasmid or dsDNA controls at time x)}*100. Larger values show more efficient condensation of genetic material into nanoparticles (SYBR exclusion assay). These nanoparticles are created using computer-assisted formulation design, whereby various ratios of poly(L-glutamic acid) and poly(D-glutamic acid) (PLE20 and PDE20) are evaluated and the associated physicochemical properties of single-layered nanoparticles (payload+outer layer) and multi-layered (payload+layer 1+layer 2+. . . +layer n) nanoparticles are gathered as a baseline for Cas9 nanoparticle synthesis. Shown are particles condensed with either poly(L-arginine) (PLR, n=100), or histone-derived cysteine-substituted cationic polypeptide sequence H2B-3C (CEVSSKGATICKKGFKKAVVKCA). Group B represents plasmid DNA (pDNA_mTagGFP2-N1), while Group E represents linear DNA (dsDNAmTagGFP2-N1). Each component had a charge ratio of 3:1 and the anionic polymer consisted of PLE20 and PDE20.

FIG. 19D depicts particle sizes of nanoparticles synthesized via computer-assisted formulation design, whereby various ratios of poly(L-glutamic acid) and poly(D-glutamic acid) are evaluated and the associated physicochemical properties of single-layered nanoparticles (payload+outer layer) and multi-layered (payload+layer 1+layer 2++layer n) nanoparticles are gathered as a baseline for Cas9 nanoparticle synthesis. Shown are particles condensed with either poly(L-arginine) (PLR50), or histone-derived cysteine-substituted cationic polypeptide sequence H2B-3C (CEVSSKGATICKKGFKKAVVKCA). Particle sizes were measured via a Wyatt Mobius Zeta Potential and DLS Detector.

FIG. 19E depicts zeta potentials of nanoparticles synthesized via computer-assisted formulation design, whereby various ratios of poly(L-glutamic acid) and poly(D-glutamic acid) are evaluated and the associated physicochemical properties of single-layered nanoparticles (payload+outer layer) and multi-layered (payload+layer 1+layer 2++layer n) nanoparticles are gathered as a baseline for Cas9 nanoparticle synthesis. Shown are particles condensed with either poly(L-arginine) (PLR50), or histone-derived cysteine-substituted cationic polypeptide sequence H2B-3C (CEVSSKGATICKKGFKKAVVKCA). Particle zeta potentials were measured via a Wyatt Mobius Zeta Potential and DLS Detector.

FIG. 19F1 depicts computer-assisted formulation design. The table's values represent volume (IL) of the respective solution, whereby a robotic fluid handling system executes the instructions from left to right. Subsequent physicochemical and biological studies examined dsDNA condensation with various ratios of poly(L-glutamic acid) and poly(D-glutamic acid) (PLE20 and PDE20) and applied to a Cas9 ribonucleoprotein (RNP) condensation experiment with either NLS-Cas9-2NLS with a LL236 gRNA (targeting TRAC locus), or NLS-Cas9-EGFP with a LL224 gRNA (targeting TRAC locus). The associated physicochemical and biological properties of nanoparticles are to assess performance of each formulation. Shown are particles condensed with various charge ratios (CR) of 9R-PEG-CD8 ligand or mPEG5K-PLK30. CRX-Y indicates the charge ratio of cationic polypeptides (X) vs. the respective formulation breakdown on the right (Y=1-4).

FIG. 19F2 depicts representative associated formulations corresponding to the embodiment of FIG. 19F1.

FIG. 19G depicts particle sizes (nm) of formulations depicted in FIG. 19F1-2.

FIG. 19H depicts zeta potentials (mV) of formulations depicted in FIG. 19F1-2.

FIG. 19I depicts ICE scores and knockout efficiencies as determined via Sanger sequencing of the TRAC locus. Cutting efficiencies are low prior to a further round of optimization. LL236 gRNA was utilized in this study.

FIG. 19J depicts 8 computer-assisted formulation design for interrogating optimal orders of addition for forming Cas9 RNP particles.

FIG. 19K depicts optimized nanoparticle behavior in serum (constant negative zeta potential and size over time). This particular formulation utilized an EGFP-RNP, histone H2A-3C fragment, PLE20, and PLR10. Nanoparticles were incubated in serum and sampled for DLS and zeta potential measurements over 6 h.

FIG. 19L depicts how ICE and knockout scores from a subsequent round of computer-assisted formulation design and iteration around CRISPR Cas9 RNP mediated editing of the TRAC locus in human primary pan-T cells have improved vs. the embodiments in FIG. 19I, but remain <10% for all formulations tested.

FIG. 19M depicts computer-assisted formulation design, whereby results of dsDNA condensation (19B) and Cas9 RNP condensation (19F) with various ratios of poly(L-glutamic acid) and poly(D-glutamic acid) (PLE20 and PDE20) are applied to a subsequent iteration of Cas9 ribonucleoprotein (RNP) condensation experiments with either NLS-Cas9-2NLS with a LL236 gRNA (targeting TRAC locus), or NLS-Cas9-EGFP with a LL224 gRNA (targeting TRAC locus). The associated physicochemical and biological properties of nanoparticles are to assess performance of each formulation. Shown are particles condensed with various charge ratios (CR) of H2A-3C, H2B-3C, PLR10, PLR50, and PLR100, with either PLE20 or PLE20/PDE20 (1:1). CR10 and 20 indicate cationic to anionic charge ratios, whereas PLE concentrations are held constant (2:1−/+electrostatic layering ratio). The final cationic ligand layer had a +/−3:1 electrostatic layering ratio.

FIG. 19N depicts computer-assisted formulation design, whereby results of dsDNA condensation (19B) and Cas9 RNP condensation (19F) with various ratios of poly(L-glutamic acid) and poly(D-glutamic acid) (PLE20 and PDE20) are applied to a subsequent iteration of Cas9 ribonucleoprotein (RNP) condensation experiments with either NLS-Cas9-2NLS with a LL236 gRNA (targeting TRAC locus), or NLS-Cas9-EGFP with a LL224 gRNA (targeting TRAC locus). This table displays the degrees of freedom studied from this particular permutation of optimized core template vs. anionic layer vs. cationic anchor-ligand, and the associated basis for forming robotic fluid handling instructions. The associated physicochemical and biological properties of nanoparticles are to assess performance of each formulation. Shown are particles condensed with various charge ratios (CR) of H2A-3C, H2B-3C, PLR10, PLR50, and PLR100, with either PLE20 or PLE20/PDE20 (1:1). CR10 and 20 indicate cationic to anionic charge ratios, whereas PLE concentrations are held constant (2:1-1+electrostatic layering ratio). The final cationic ligand layer had a +/−3:1 electrostatic layering ratio.

FIG. 19O depicts particle sizes of each associated formulation in FIGS. 19M-19N.

FIG. 19P depicts zeta potentials of each associated formulation in FIGS. 19M-19N.

FIG. 19Q depicts Sanger sequencing and ICE (inference of CRISPR edits) analysis of representative nanoparticle groups in human primary Pan T cells, comparing stimulated (top) and unstimulated T cells (bottom) transfected without serum. C11-F11 depict nucleofection positive controls. Up to 34% TRAC editing efficiency was achieved with nanoparticle-mediated unstimulated T cell delivery, vs. 34, 40, 63 and 70% for nucleofection controls. Additionally, up to 22% TRAC editing efficiency was achieved with nanoparticle-mediated stimulated T cell delivery vs. 10, 14, 20 and 37% for nucleofection controls.

FIG. 19R depicts Sanger sequencing and ICE (inference of CRISPR edits) analysis of representative nanoparticle groups in human primary Pan T cells, comparing stimulated (bottom) and unstimulated (top) T cells. Note: Arrows indicate positive controls (nucleofection). Once nanoparticle cores have been iterated and consolidated for a certain payload, a similar iteration process follows for the nanoparticle ligand surface based on the specific cell of interest. In the following example, different surface ligands were iterated over to target either T cells generally, or subpopulation of T cells such as CD4+ or CD8+specifically.

FIG. 19S depicts a multiparametric data visualization of biological and physicochemical results of nanoparticles transfected into human primary pan-T cells. Shown from left to right are ICE scores, knockout scores, % of cells alive & non-apoptotic, % of live cells containing nanoparticles (based on flow cytometry measuring cell inclusion of 0.1% w/w inclusion of Endo_XAlexa594_4 G-5_3 KRK_2_N_1 (c124)), and particle sizes (nm). Particle formulations may be rapidly permutated through in this way and with other structured and unstructured machine learning approaches as detailed elsewhere.

FIG. 19T depicts robotic formulations for multilayered nanoparticles performed by an Andrew liquid handling robot, as designed by the formulator app and corresponds to FIG. 19V. Values represent microliters of fluid handled by the robot and moved to the given well location.

FIG. 19U depicts continued robotic formulations for multilayered nanoparticles performed by an Andrew liquid handling robot, as designed by the formulator app and corresponds to FIG. 19E. Values represent microliters of fluid handled by the robot and moved to the given well location.

FIG. 19V depicts several rounds of screening CRISPR RNP bearing nanoparticles. Single-layered and multi-layered nanoparticles exhibit clusters of sizes that display ideal physicochemical properties for transfection of human primary T cells (human Pan-T Cells, which include CD4+ and CD8+ subtypes). This demonstrates Iterative cell-specific ligand design for T cells (CD4+ and CD8+ Pan-T cells) whereby individual ligands are interrogated and optimized at various densities and with various core templates. This allows for ligands to be modularly studied upon a variety of core chemistries and polymer/polypeptide compositions, as well as various payloads. Compared to the heteromultivalent studies (where a global optimal was found for a static set of targeting ligand densities, e.g. anchor cationic interactions with anionic payload), these results show that further core optimization may also achieve optimization of cellular uptake and affinity of ligands for various cell subpopulations. Many of the optimized cores are based on prior optimization work (see HSC-directed nanoparticles) whereby multilayering strategies may be used (e.g. ligands are patterned upon a cationic and/or anionic polymer stabilizing layer). Shown are comparisons of single-layered (ligands directly added to payload) vs. multi-layered (ligands added to core particles) and corresponding T cell uptake efficiencies. In this example, the peptide sequence corresponding to Endo_XAlexa594_4GS_3KRK_2_N_1 is utilized at 0.01% w/v on the particle surface in addition to varying core and ligand compositions shown across the plate. The corresponding sequence is: KKKRKKKKRKGGGGSC(AF594)GGGGSSFKFLFDIIKKIAES. Transfection efficiency was evaluated via flow cytometry (Attune NxT flow cytometer) 1 d post-transfection. In this example, this peptide demonstrates variable transfection efficiency of a variety of complexes without acting as a direct ligand itself, suggesting that the alternative chemistries used to design the nanoparticles (core, multilayering and ligand variability), rather than a “non-complexed fluorescently-tagged ligand” that is not formed with a nanoparticle, lead to the increases in fluorescence uptake (AF594+ cells) in these studies of various nanoparticle compositions. In alternative embodiments, a targeting ligand may include similar fluorophore modifications on one or more cysteine residues (or through alternative coupling techniques) in order to track individual ligand binding to cellular receptor profiles prior to inclusion in nanoparticles or conjugation to small molecule drugs/biologics/etc.

FIG. 19W depicts a continuation of the previous figure exhibiting CRISPR RNP delivery. Single-layered nanoparticles (ligand or cationic polypeptide directly added to RNP payload) are shown on the right, whereas multi-layered nanoparticles (core formed from cationic and/or anionic polymers prior to coating in an oppositely-charged ligand anchor) are shown on the right. This figure demonstrates iterative cell-specific ligand design whereby individual ligands are interrogated and optimized at various densities and with various core templates. This allows for ligands to be modularly studied upon a variety of core chemistries and polymer/polypeptide compositions, as well as various payloads. Compared to the heteromultivalent studies (where a global optimal was found for a static set of targeting ligand densities, e.g. anchor cationic interactions with anionic payload or vice versa), these results show that further core optimization may also achieve optimization of cellular uptake and affinity of single ligands for various cell subpopulations. Ligand-coated complexes outperform cell-penetrating peptide coated complexes. These nanoparticle variants also demonstrate up to 94% efficient CD4+ T cell and 68% efficient CD8+ T cell transfection of CRISPR RNPs, as measured by AF594+ cells, into live subpopulations (see well H7), and many variants with ˜10× selectivity for CD4 subpopulations vs. CD8 subpopulations (see well locations A4-H5 for multi-layered and A6-H8 single-layered particles). Despite a single ligand being used (either CD4 or CD8 ligand or cell-penetrating peptide), optimization of core and nanoparticle surface presentation of the ligands resulted in enhanced uptake versus heteromultivalent screens with suboptimal cores. Multilayered nanoparticles demonstrably showed enhanced transfection efficiency and uptake in live T cell subpopulations versus single-step assembly variants.

FIG. 19X depicts a continuation of the previous figure exhibiting CRISPR RNP delivery. This demonstrates iterative cell-specific ligand design whereby individual ligands are interrogated and optimized at various densities and with various core templates. This allows for ligands to be modularly studied upon a variety of core chemistries and polymer/polypeptide compositions, as well as various payloads. Compared to the heteromultivalent studies (where a global optimal was found for a static set of targeting ligand densities, e.g. anchor cationic interactions with anionic payload or vice versa), these results show that further core optimization may also achieve optimization of cellular uptake and affinity of single ligands for various cell subpopulations. Ligand-coated complexes outperform cell-penetrating peptide coated complexes. These nanoparticle variants also demonstrate up to 94% efficient CD4+ T cell and 68% efficient CD8+ T cell transfection of CRISPR RNPs into live subpopulations (see well H7), and many variants with ˜10× selectivity for CD4 subpopulations vs. CD8 subpopulations (see well locations A4-H5).

FIG. 19Y depicts a continuation of the previous figure exhibiting CRISPR RNP delivery via a number of nanoparticle formulations. Shown here are particle sizes of each respective single-layered nanoparticle formulation. PLK10-PEG22 and PLR10 particles with variable endosomal escape peptide/functional domain peptide (EE) concentrations are shown to condense NLS-Cas9-NLS, but not NLS-Cas9-EGFP, into sub-50-nm particles at 3 orders of addition of EE vs. cationic polypeptide groups (wells A9-H10 and D12-E12). These particle sizes are demonstrably smaller than RNP-only sizes, and suggest the role of short (<20 AA) cationic polypeptides in being able to uniquely dissociate RNP aggregates prior to subsequent multilayering or inclusion with a variety of nanoparticle formulations or alternative delivery systems (e.g. covalently modified RNPs, liposomes, and the like). We have previously demonstrated nanoparticles condensed in this way to be multilayered with either another nucleotide and PLE/PDE, or a nucleotide on its own, prior to a final layer of cationic anchor-ligand. We have also demonstrated anionic anchor-ligand groups to be able to condense around cationic layers. This screening study demonstrates iterative cell-specific ligand design whereby individual ligands are interrogated and optimized at various densities and with various core templates. Additionally, this allows for ligands to be modularly studied upon a variety of core chemistries and polymer/polypeptide compositions, as well as various payloads. Compared to the heteromultivalent studies (where a global optimal was found for a static set of targeting ligand densities, e.g. anchor cationic interactions with anionic payload or vice versa), these results show that further core optimization may also achieve optimization of cellular uptake and affinity of single ligands for various cell subpopulations. Ligand-coated complexes outperform cell-penetrating peptide coated complexes. These nanoparticle variants also demonstrate up to 94% efficient CD4+ T cell and 68% efficient CD8+ T cell transfection of CRISPR RNPs into live subpopulations (see well H7), and many variants with ˜10× selectivity for CD4 subpopulations vs. CD8 subpopulations (see well locations A4-H5).

FIG. 19Z depicts Sanger sequencing and ICE (inference of CRISPR edits) analysis of representative single-layered nanoparticle groups in human primary Pan T cells. These samples correspond to the formulations for multilayered nanoparticles in FIGS. 19V-19Y.

FIG. 19ZA depicts size considerations hypothesizing why poly(L-arginine) (n=10) and PLK10-PEG22 consistently formed CRISPR RNP nanoparticles in the 20-59 nm ranges. It is believed that PLR10 and PLK10-PEG22, which have polymer chain lengths less than the hydrodynamic diameter of Cas9 RNP, will preferentially “charge switch” the anionic components of the highly zwitterionic Cas9 RNP. Methods of using “charge switching” techniques for achieving affinity of peptide sequences to zwitterionic surfaces are also detailed in FIGS. 18T and 18U. If PLR10 or a similarly sized cationic polypeptide is able to intercalate into the anionic pockets of the zwitterionic protein, it is believed that the otherwise aggregative properties of Cas9 (presumably due to opposite charges interacting and forming electrostatic aggregates) can be reversed. These small, homogenously-charged cationic RNP-PLR10 complexes may be subsequently decorated in a variety of surface coatings, including anionic interlayers (e.g. PLE/PDE) with or without subsequent cationic anchor-linker-ligand or anchor-peptide sequences, as well as anionic anchor-linker-ligand or anchor-peptide sequences. Additionally, PLR10 serves to efficiently condense exposed sgRNA residues of the Cas9 RNP, which are anionic in nature.

FIG. 20A depicts DNA ligation based techniques for assembling TALEN sequences with site-specificity for the targeted genomic sequence. Li, Ting & Huang, Sheng & Zhao, Xuefeng & A Wright, David & Carpenter, Susan & Spalding, Martin & Weeks, Donald & Yang, Bing. (2011). Modularly assembled designer TAL effector nucleases for targeted gene knockout and gene replacement in eukaryotes. Nucleic acids research. 39. 6315-25. 10.1093/nar/gkr188.

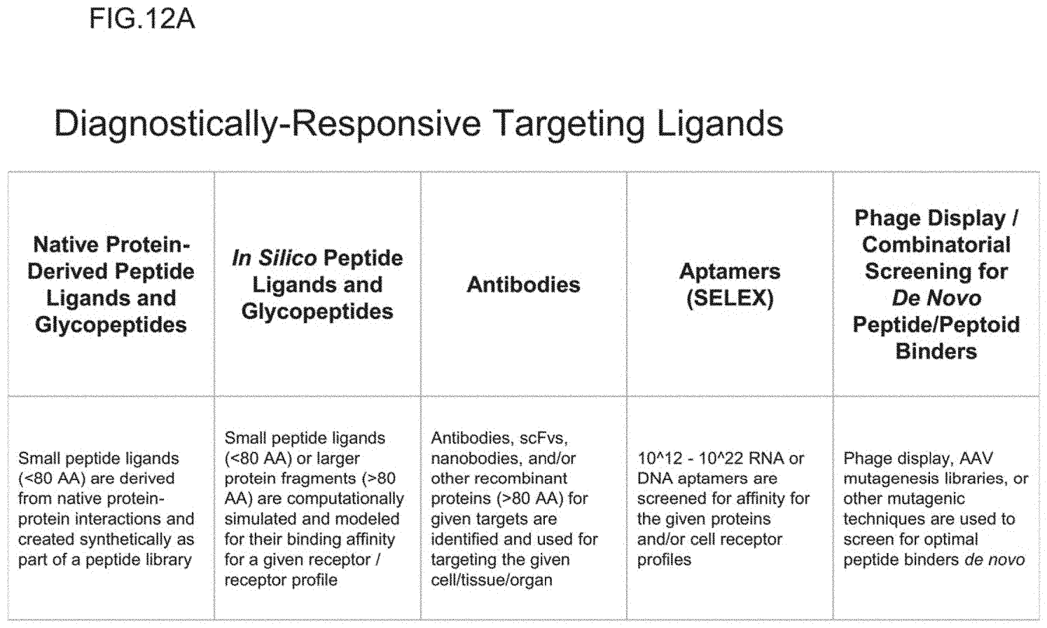

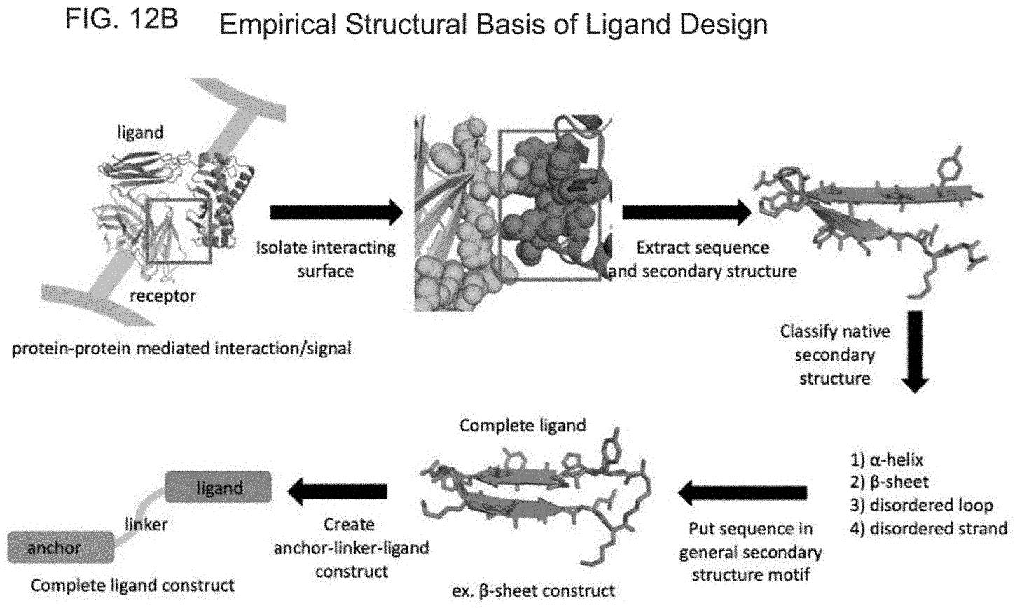

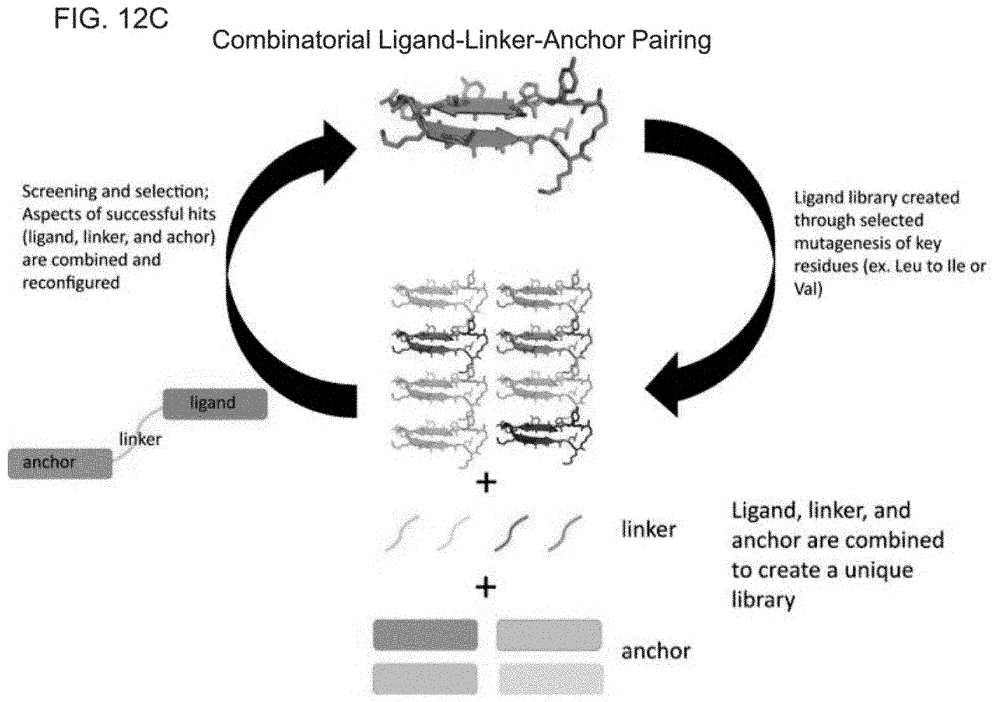

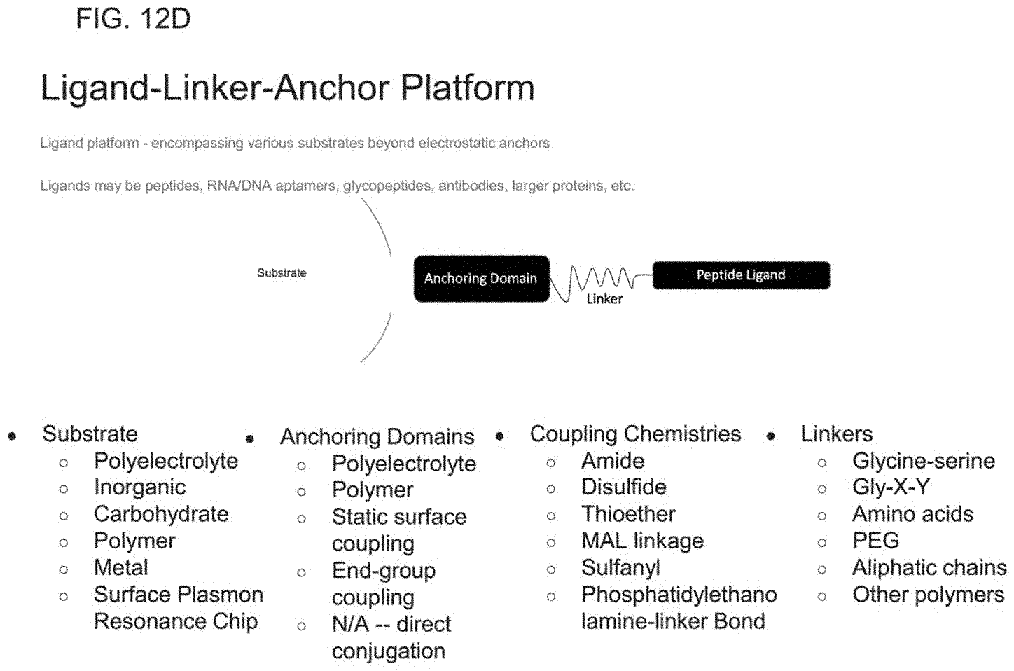

FIG. 20B depicts a protein fragment ligation based technique (native chemical ligation) for assembling TALEN or other larger recombinant-sequence-equivalent assemblies of proteins, in this instance for genome editing proteins with site-specificity for arbitrary genomic sequences. Use of synthetic peptide synthesis robots may be used to create 31-33AA fragments in ˜1 h, as well as at −100 mg scale (FIG. 22A). These 31-33A sequences of amino acids may be native chemically ligated together or otherwise paired through covalent bonding approaches. Additionally, the exposed sulfhydryl groups may serve as substrates for subsequent cysteine-bonding of anchor-linker-ligand, linker-ligand, or other ligand, charge or subcellular trafficking functionalization groups as shown in FIGS. 12A-12D. See Li, Ting & Huang, Sheng & Zhao, Xuefeng & A Wright, David & Carpenter, Susan & Spalding, Martin & Weeks, Donald & Yang, Bing. (2011). Modularly assembled designer TAL effector nucleases for targeted gene knockout and gene replacement in eukaryotes. Nucleic acids research. 39. 6315-25. 10.1093/nar/gkr188 and https://en.wikipedia.org/wiki/File:NCL_mechanism.pdf.

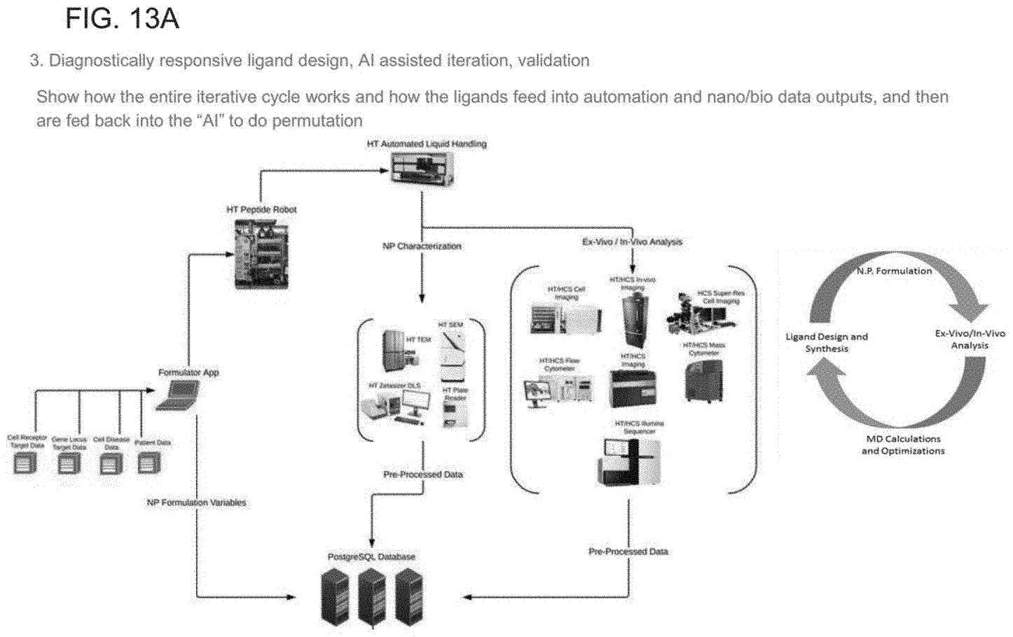

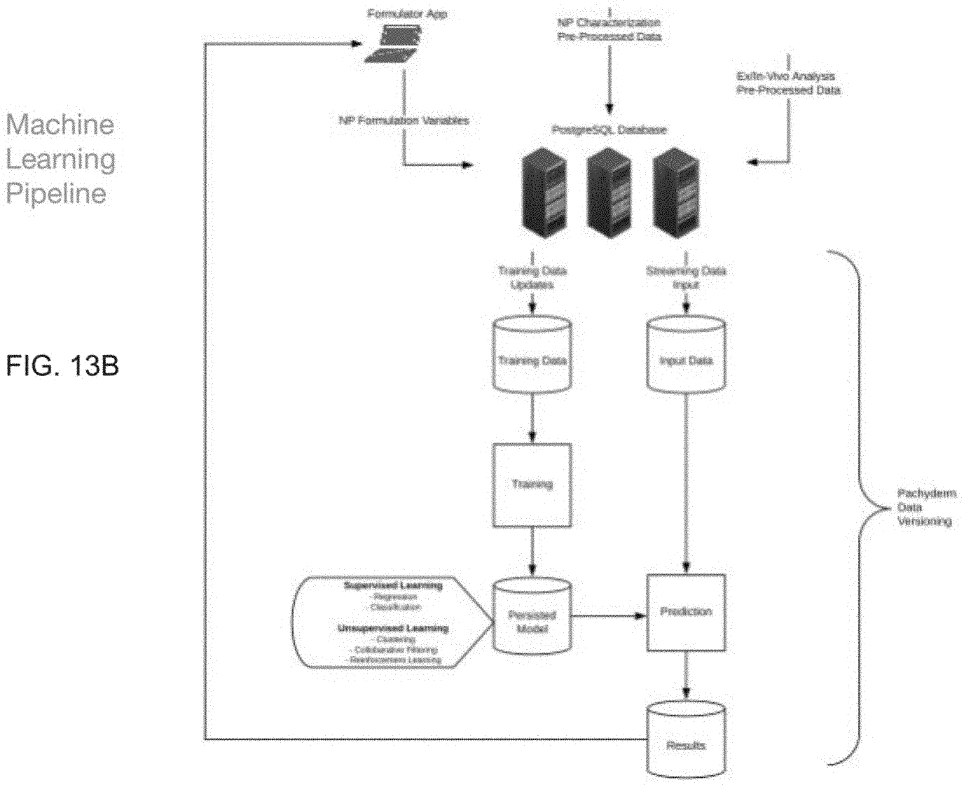

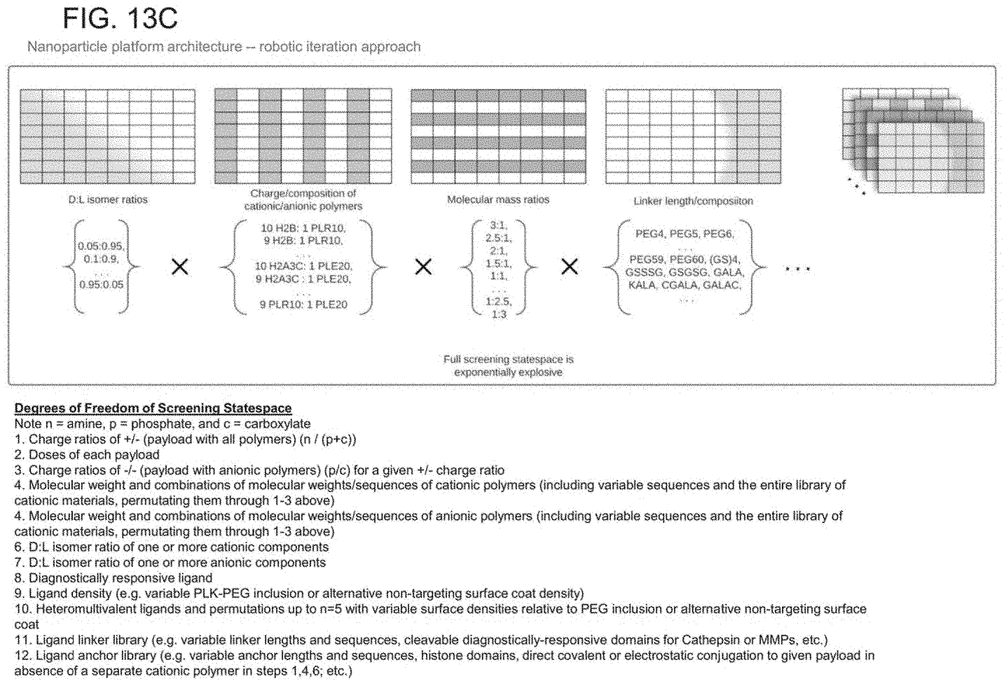

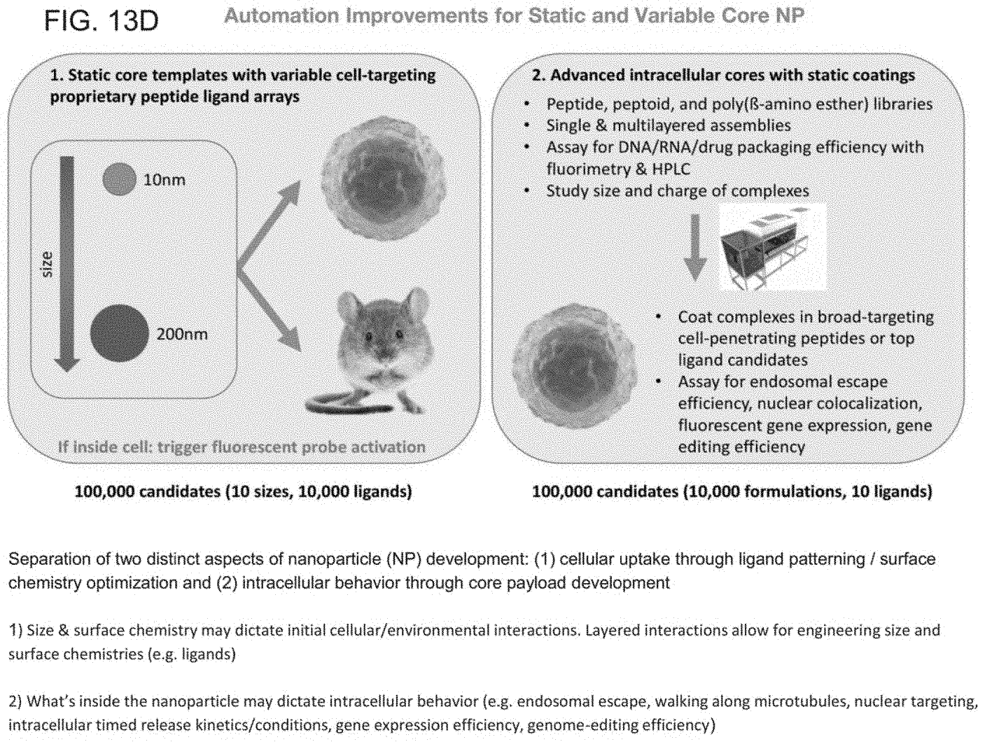

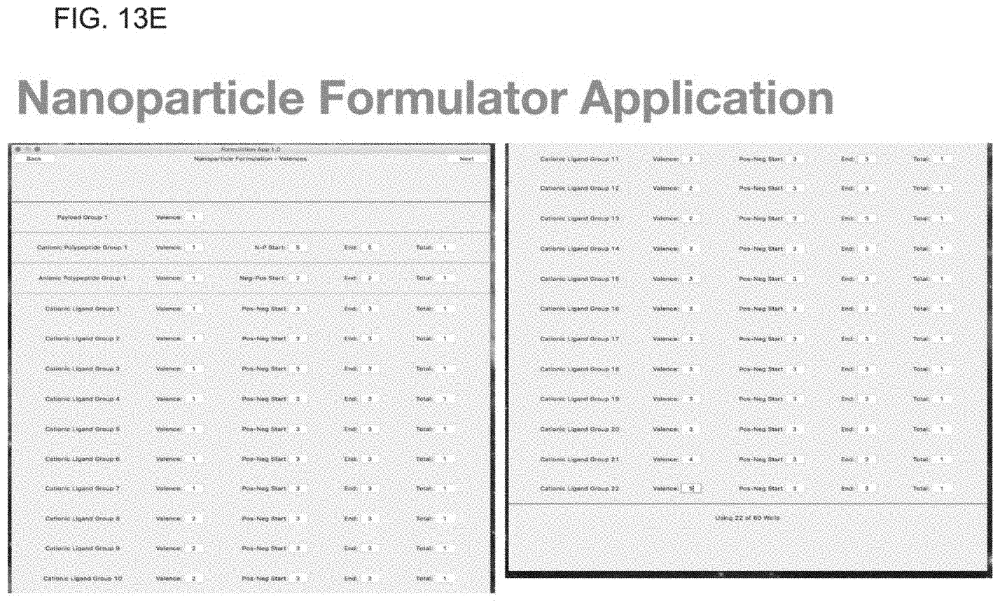

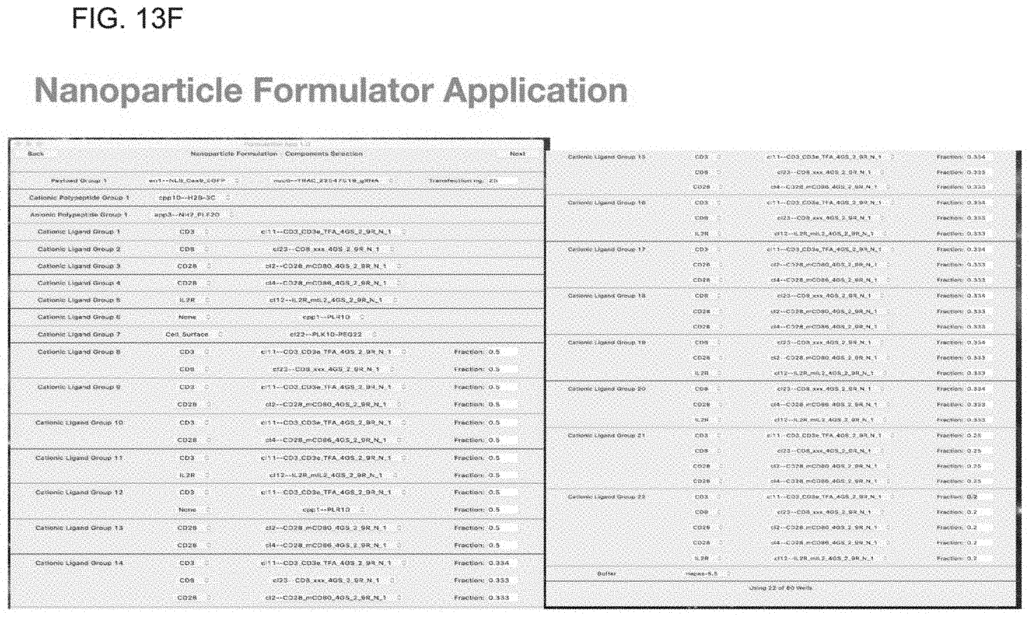

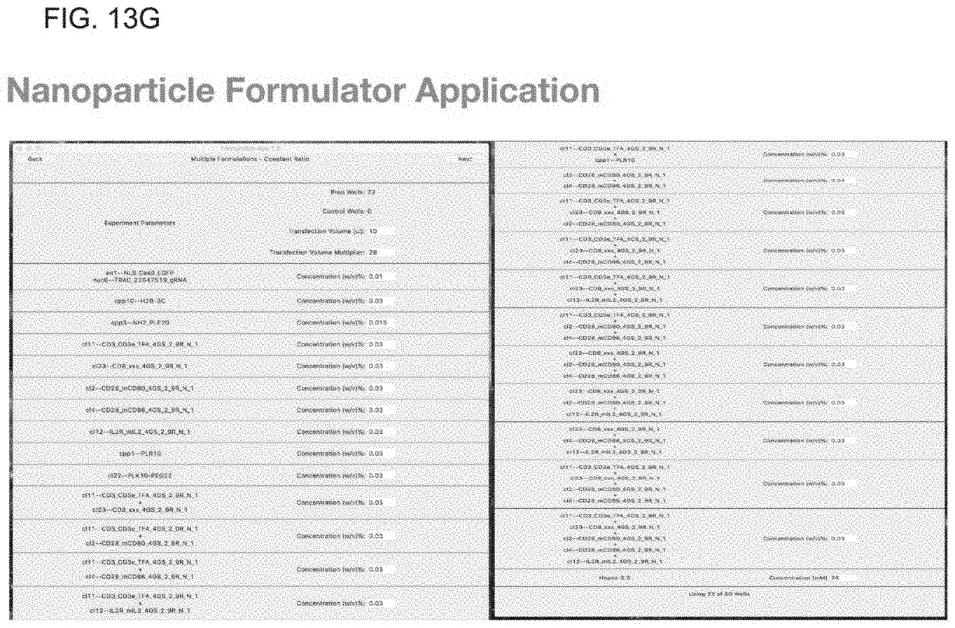

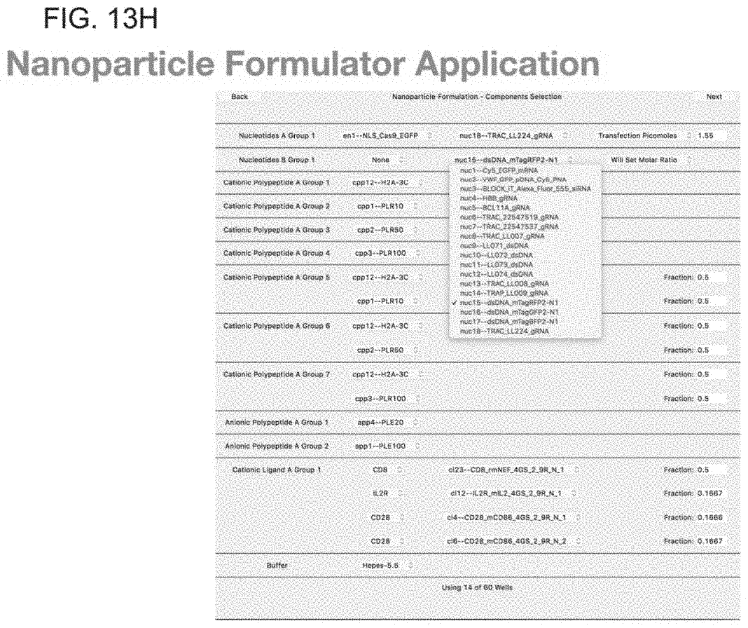

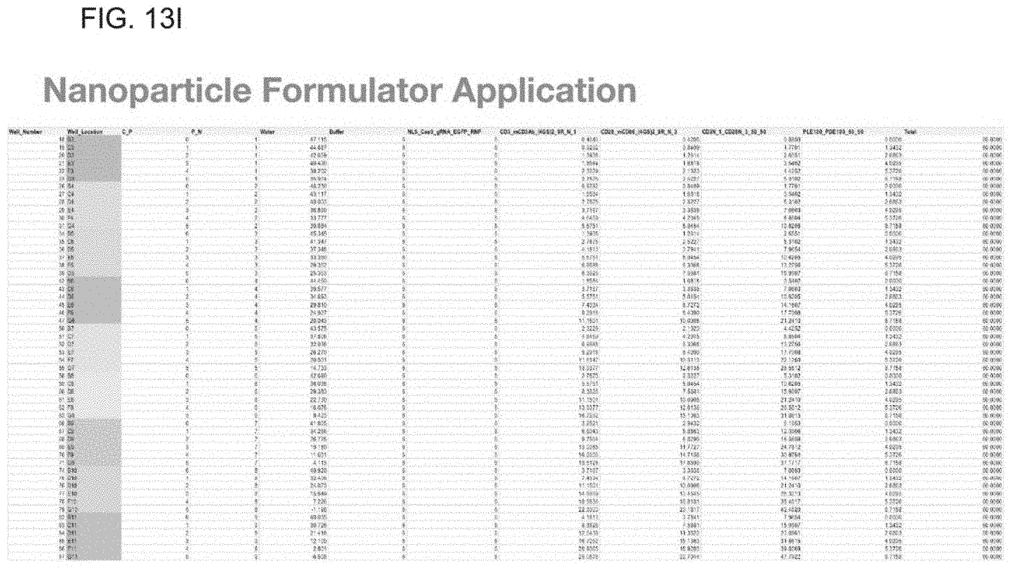

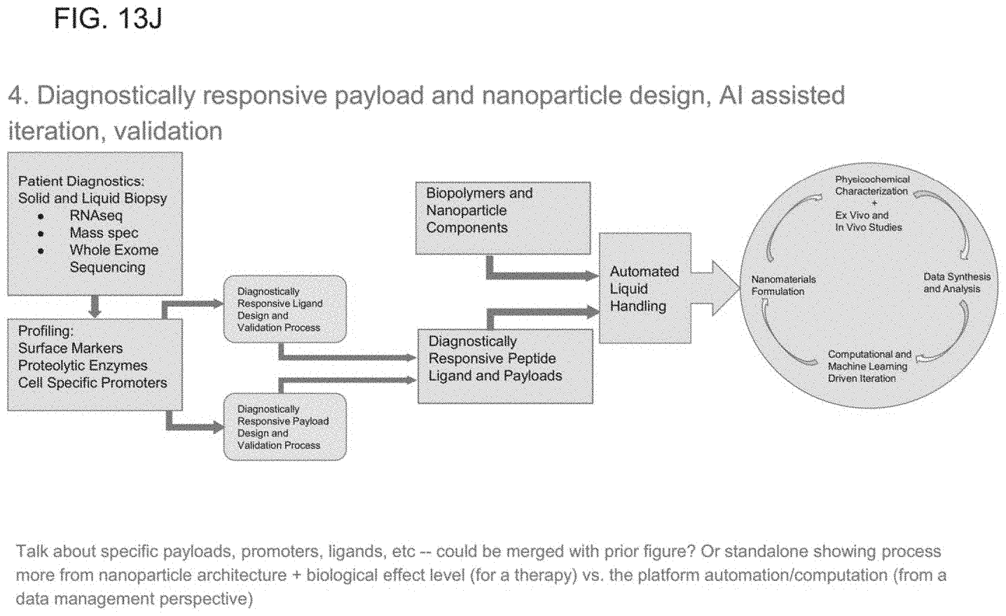

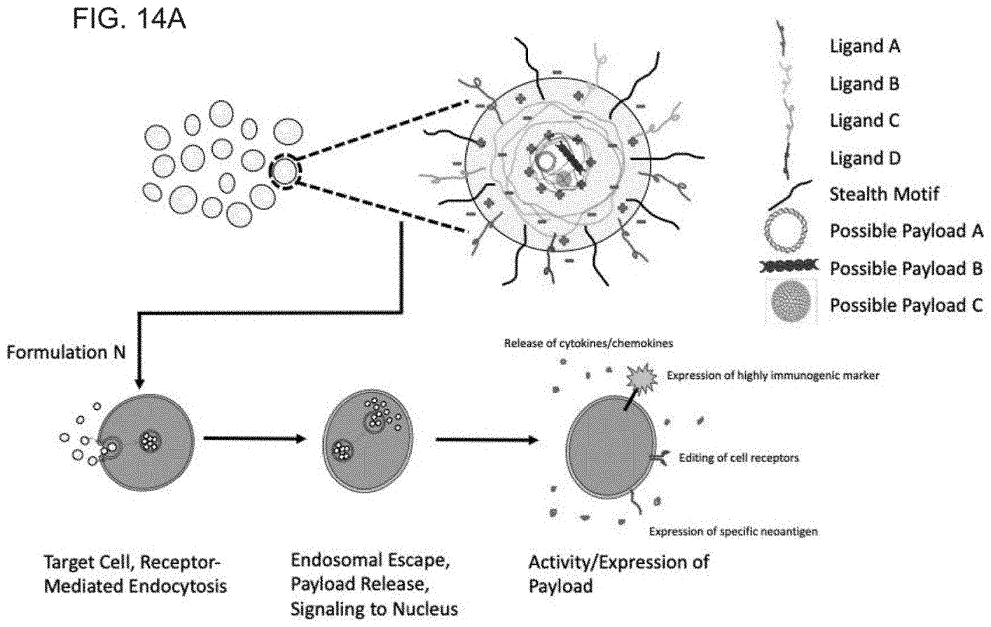

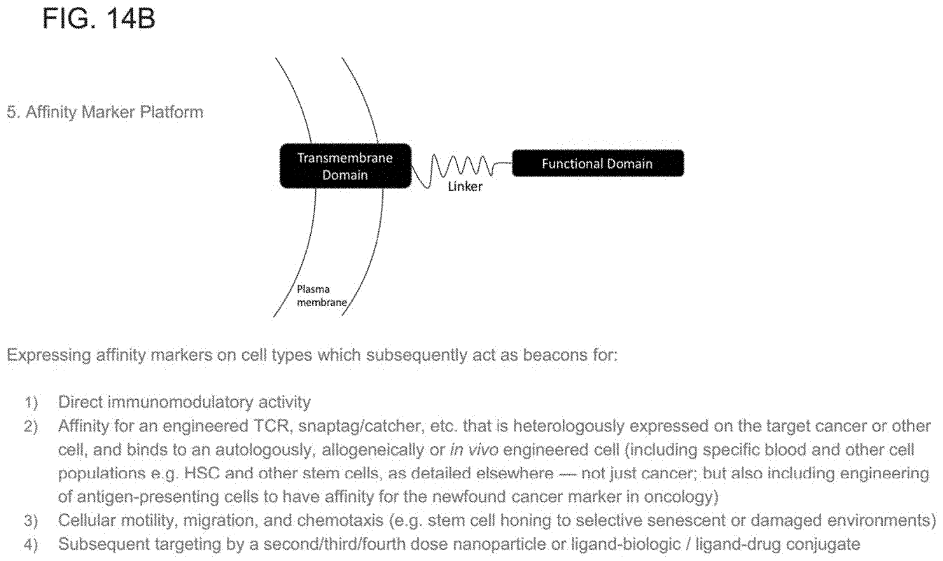

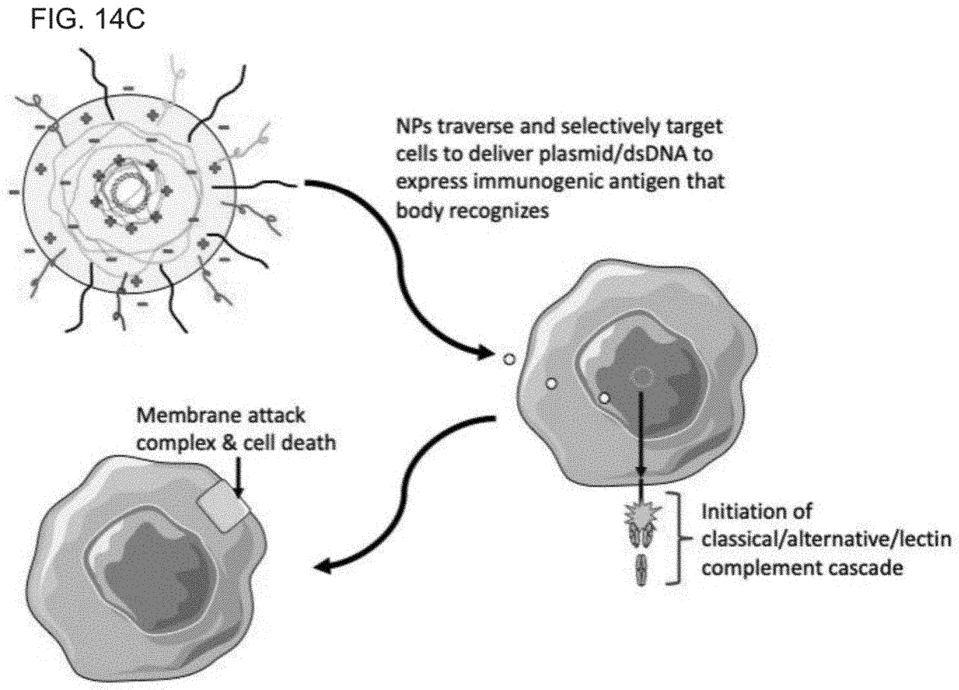

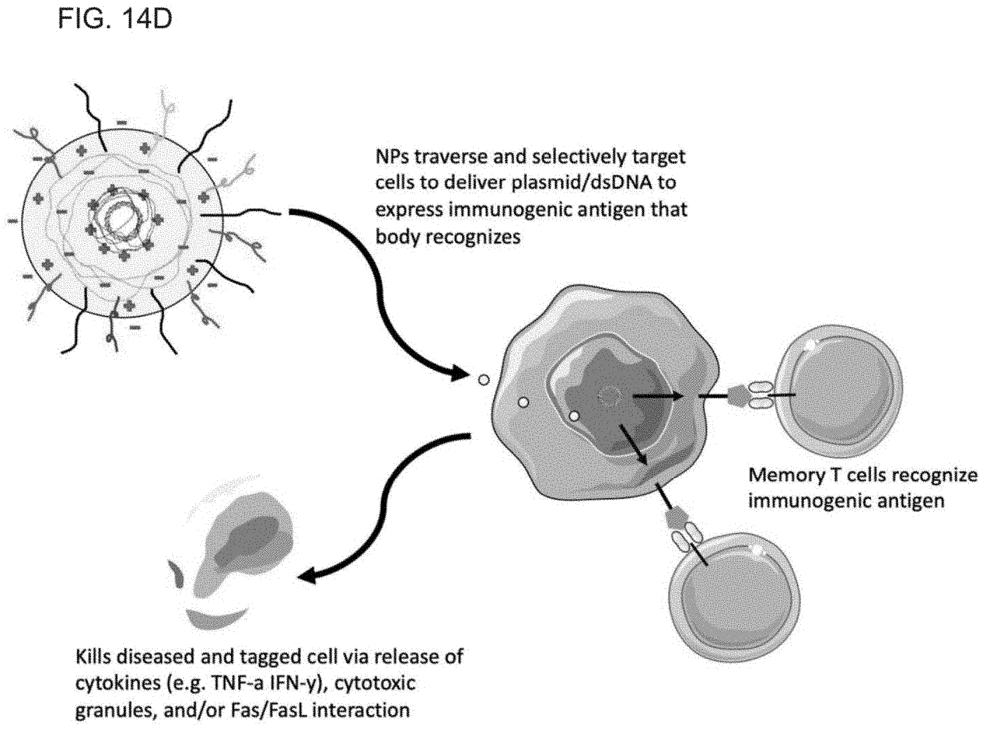

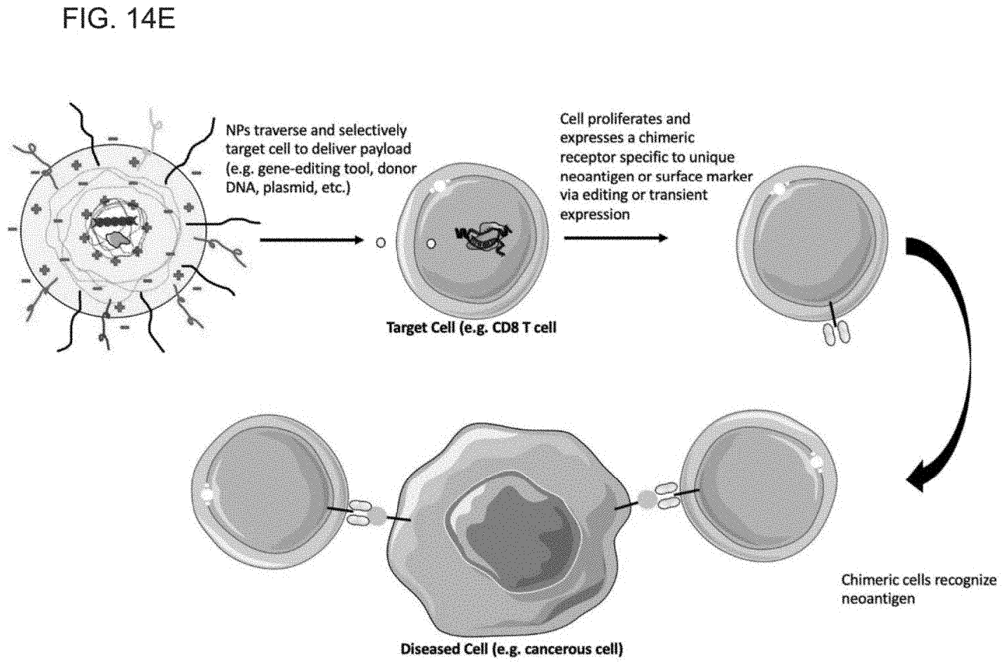

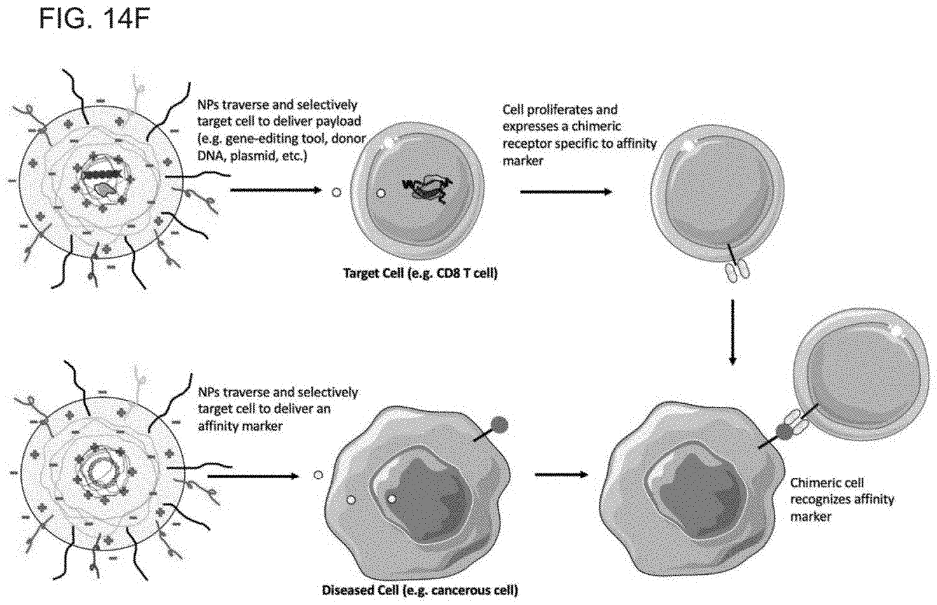

FIG. 21A depicts a flow-based peptide robotic based technique for synthesis of diagnostic-responsive targeting ligands. A single interface (shown on computer screen) can control peptide robot synthesis of diagnostically-responsive and nanoparticle-forming ligands, while a formulator app allows for customized synthesis of nanoparticle variants via Andrew robot nanoparticle synthesis as shown in FIGS. 13E-13H. The single app is also connected to an Opentrons robot programmed to perform transfections and media changes of cells (FIGS. 23B-23C).

FIG. 21B depicts ultra-rapid synthesis of an H2A-3C cationic polypeptide. Peptide synthesis of SCRGKQGCKARAKAKTRSSRCA (22AA) is completed in 55.03 minutes in an automated fashion following input of the peptide sequence into the flow-based peptide robot.

FIG. 21C depicts ultra-rapid synthesis of an H2B-3C cationic polypeptide. Peptide synthesis of CEVSSKGATICKKGFKKAVVKCA (23AA) is completed in 45.17 minutes in an automated fashion following input of the peptide sequence into the flow-based peptide robot.

FIG. 22A depicts an iPad app for performing cellular media changes and washes, as well as transfections of nanoparticles synthesized via separate robotic synthesis in FIGS. 13C-13H.

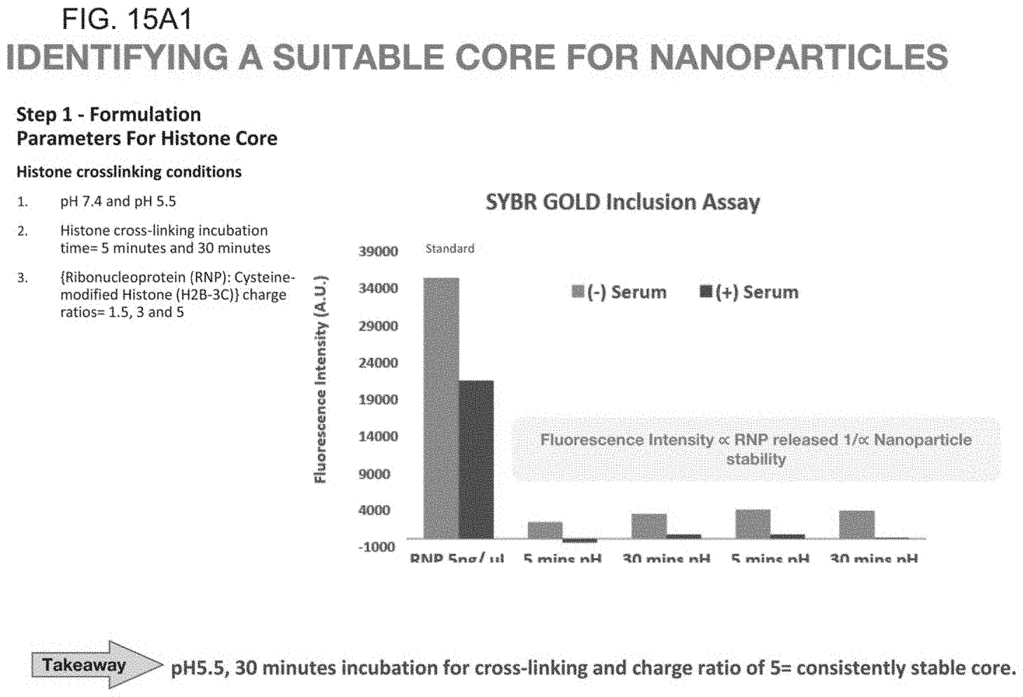

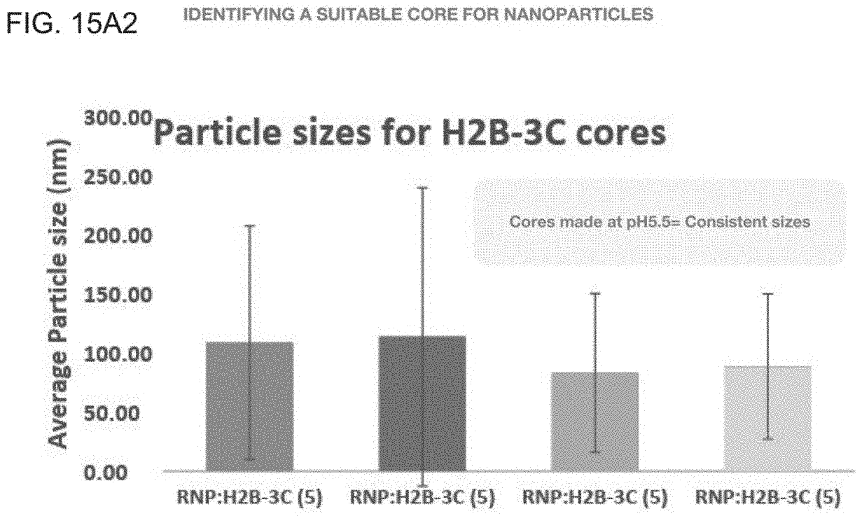

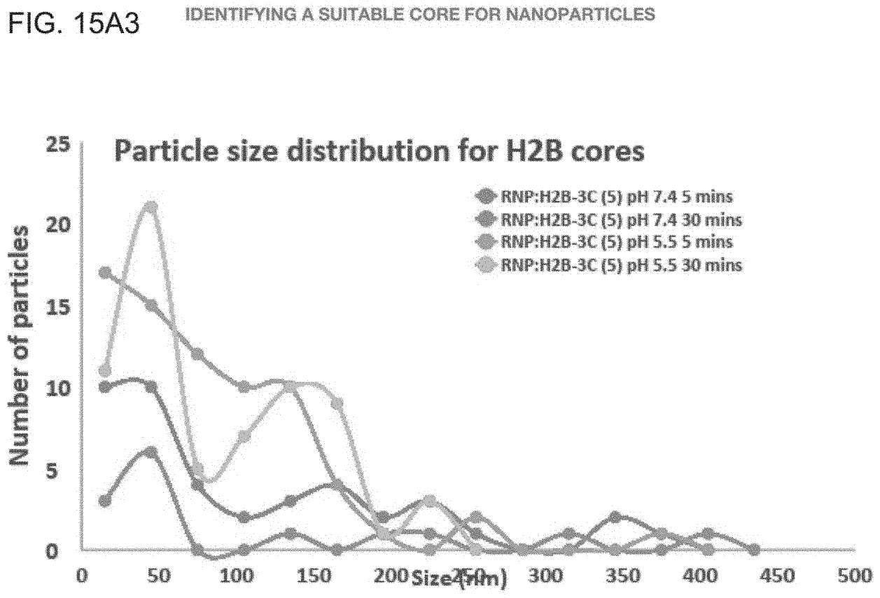

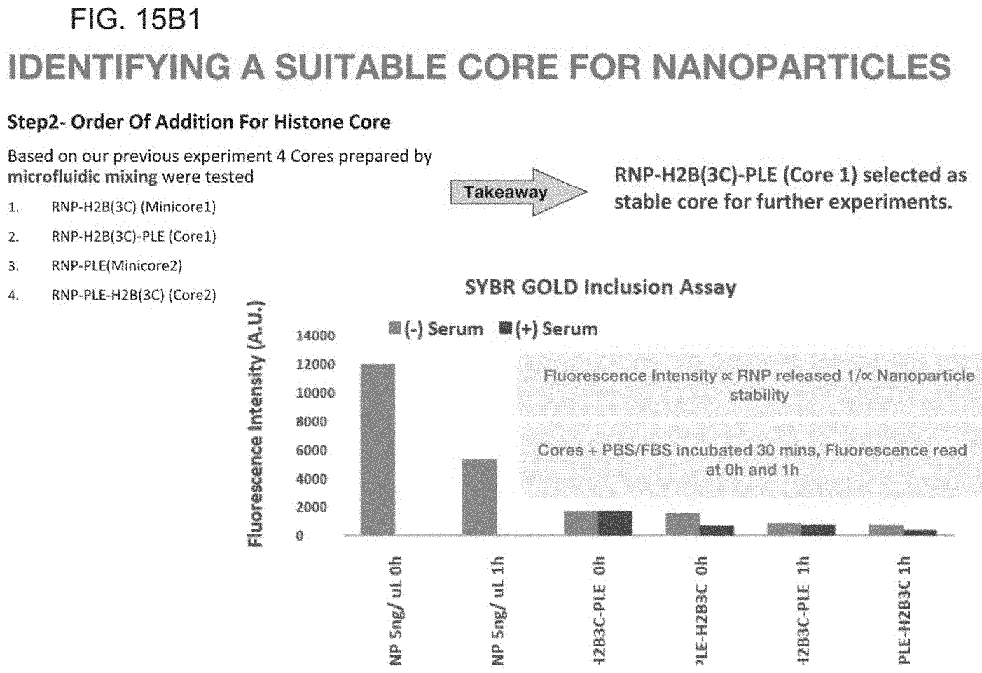

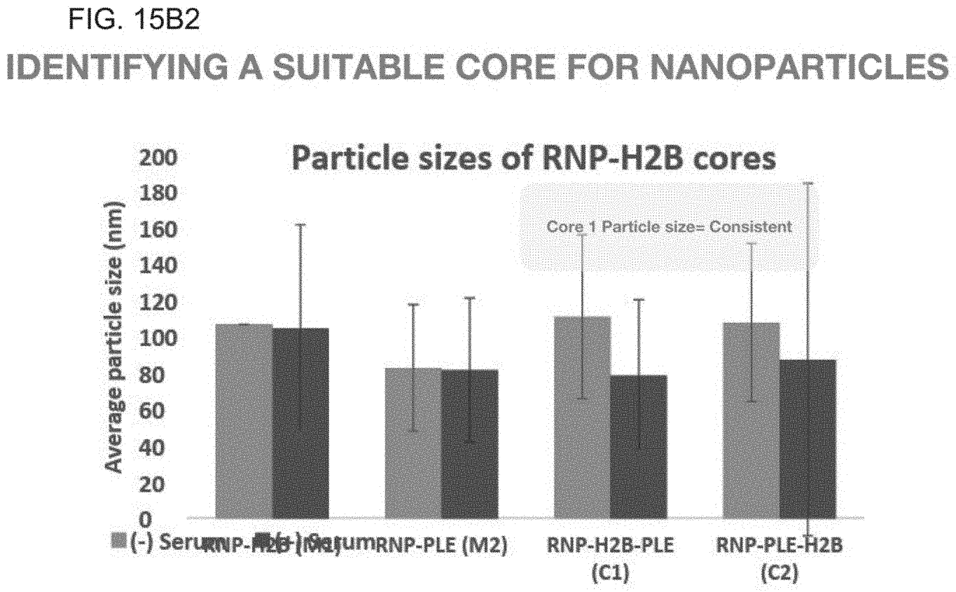

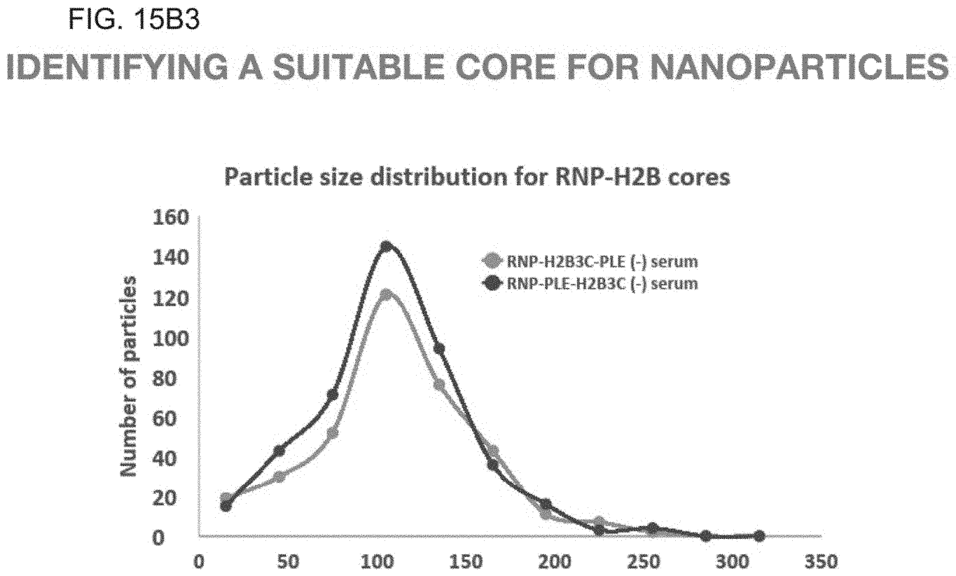

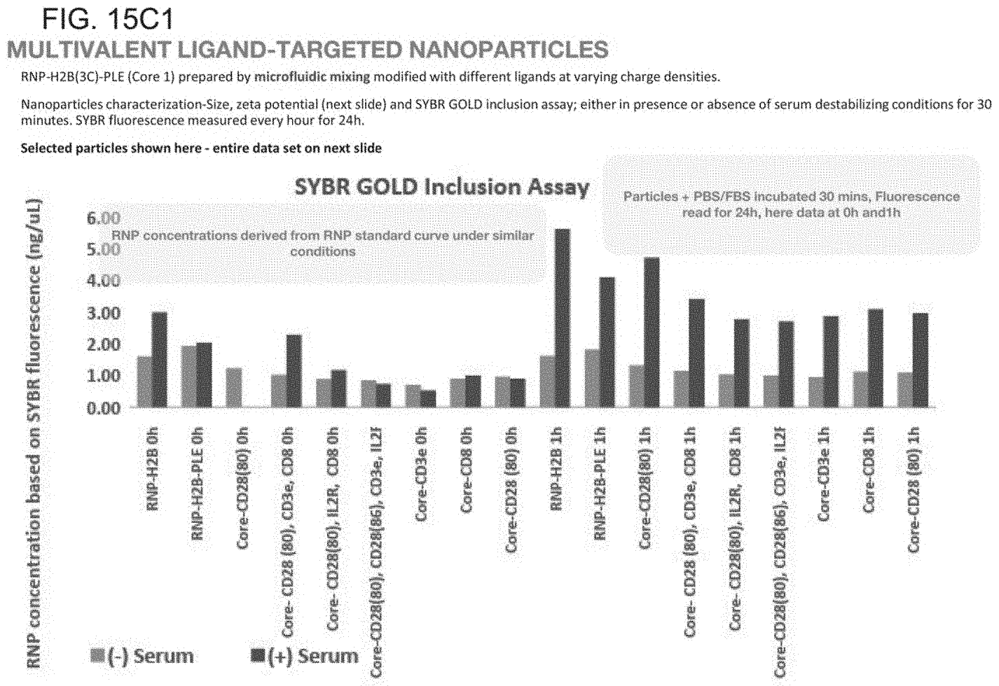

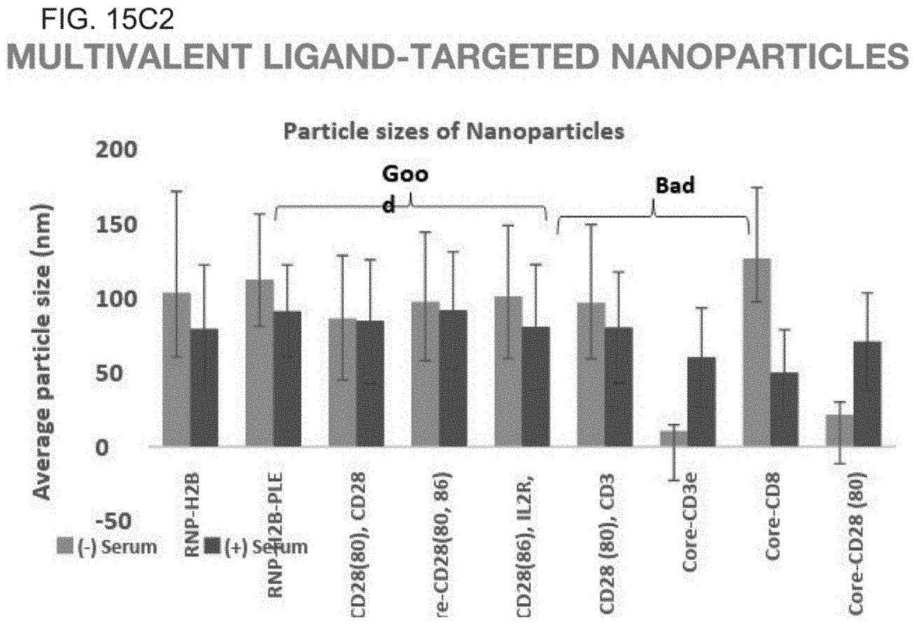

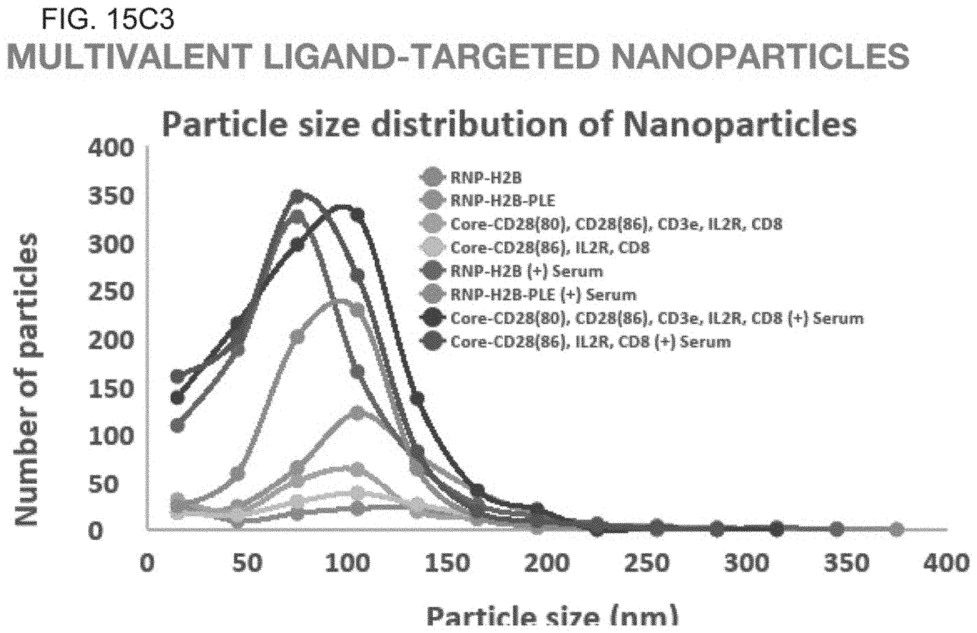

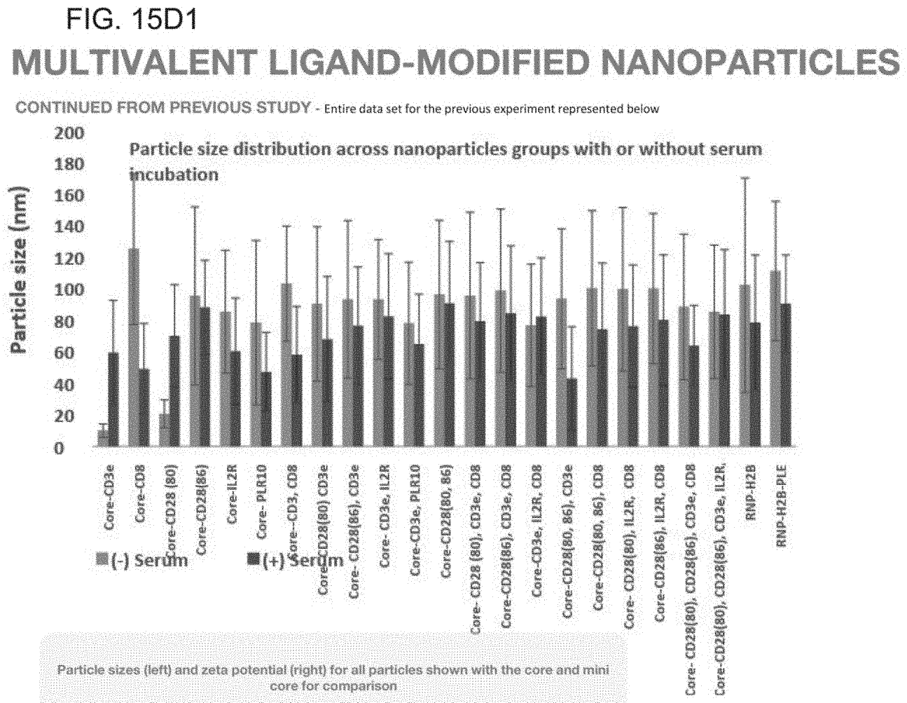

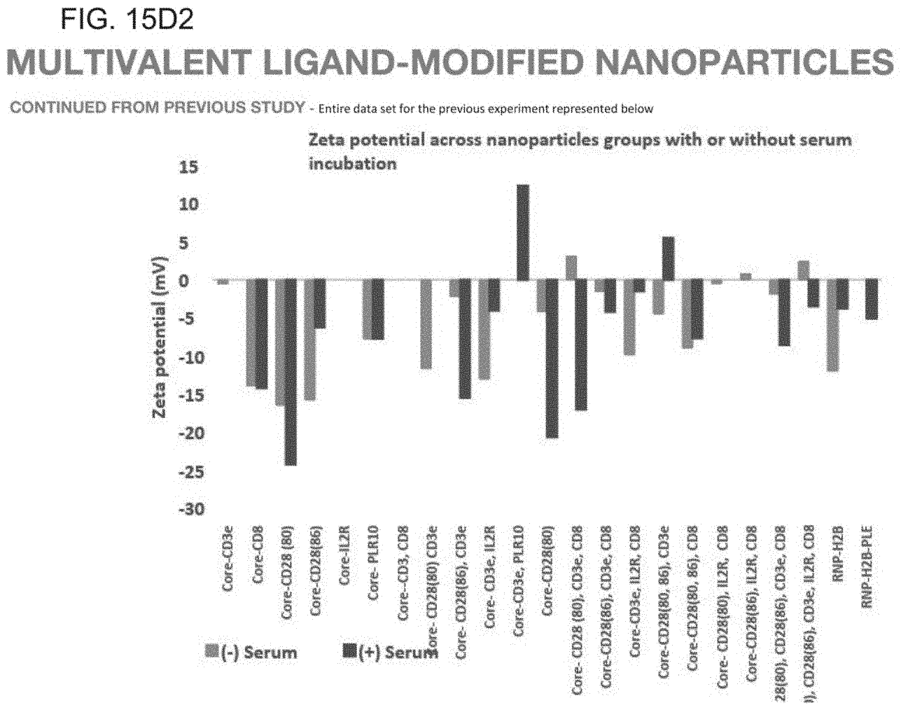

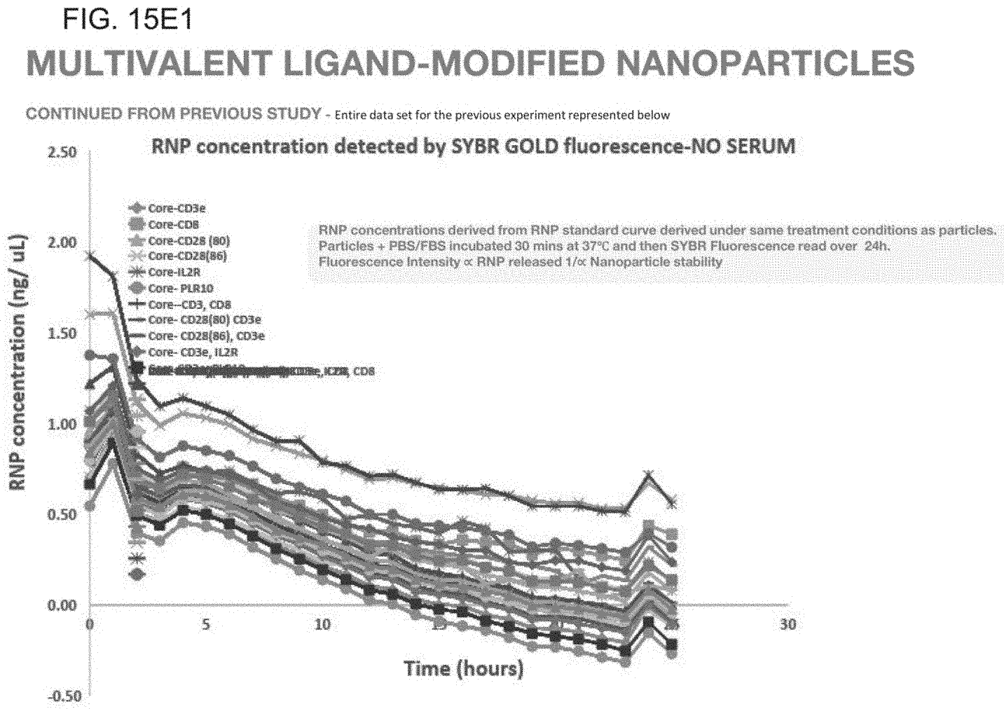

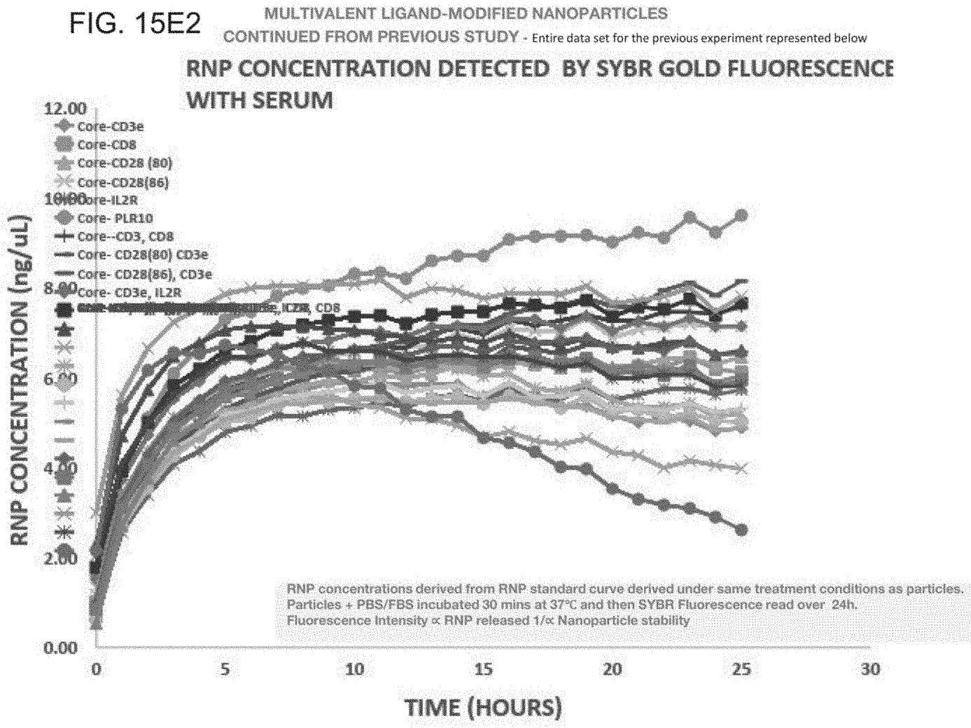

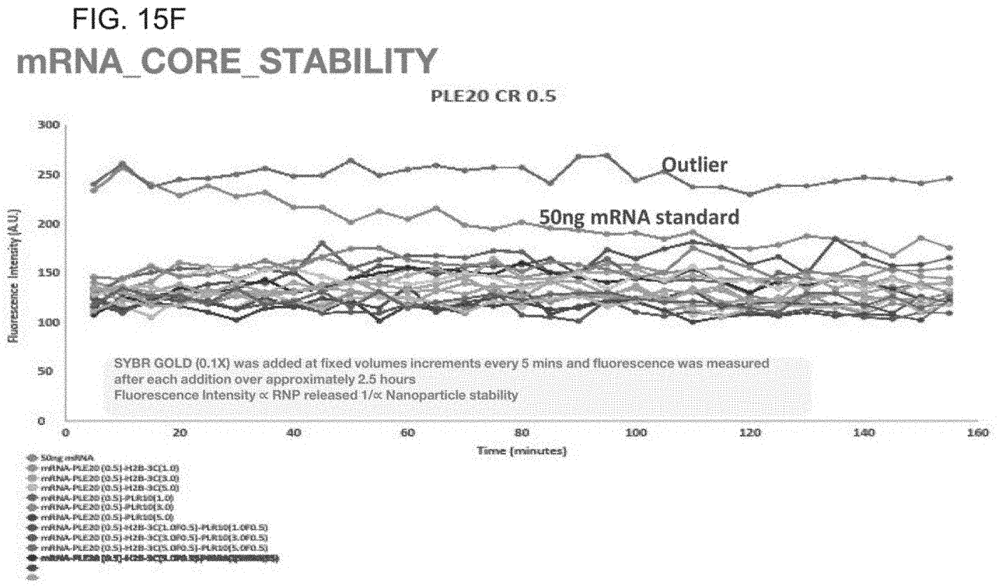

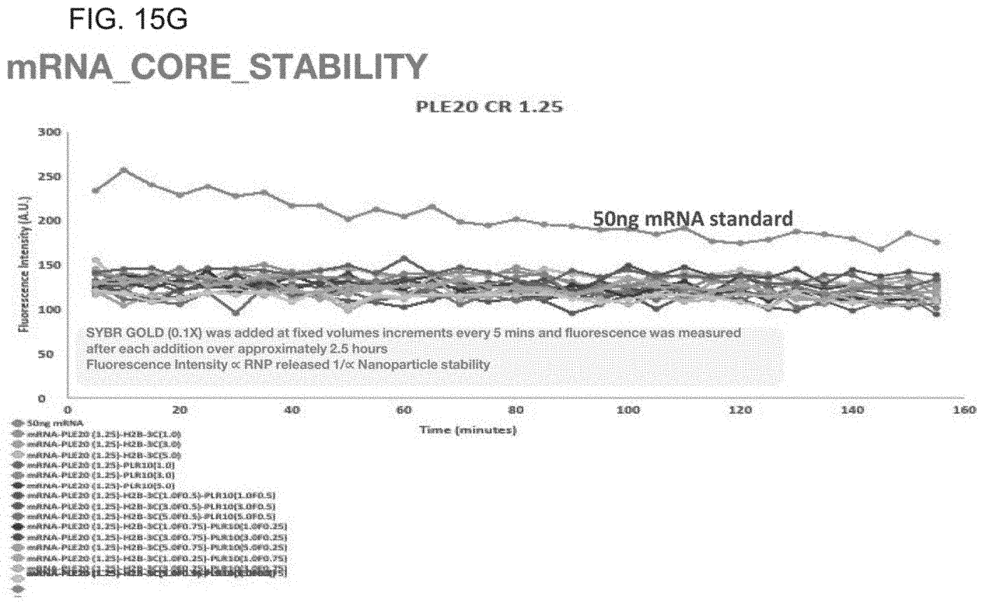

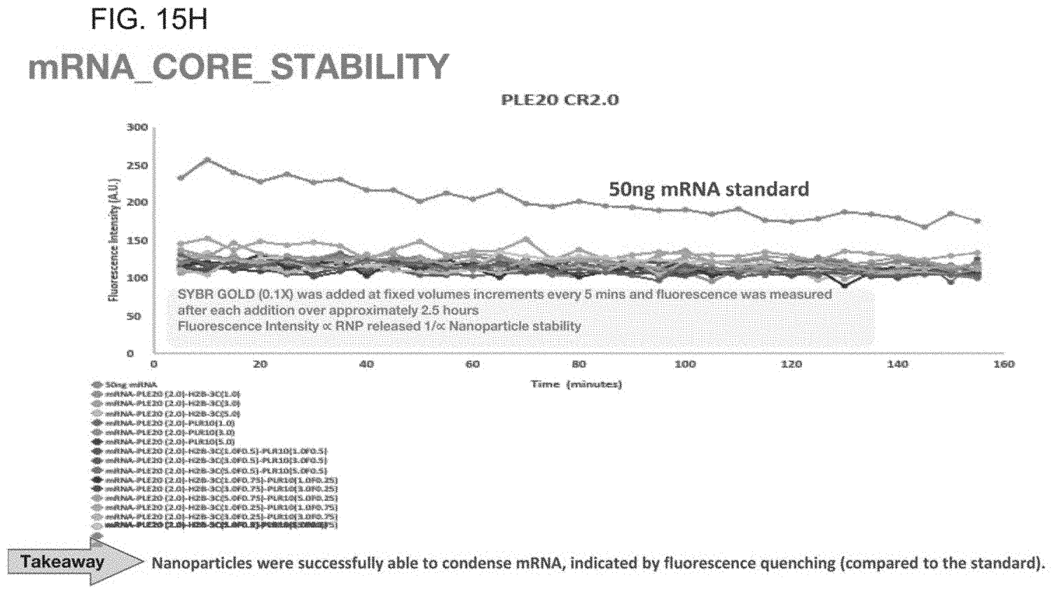

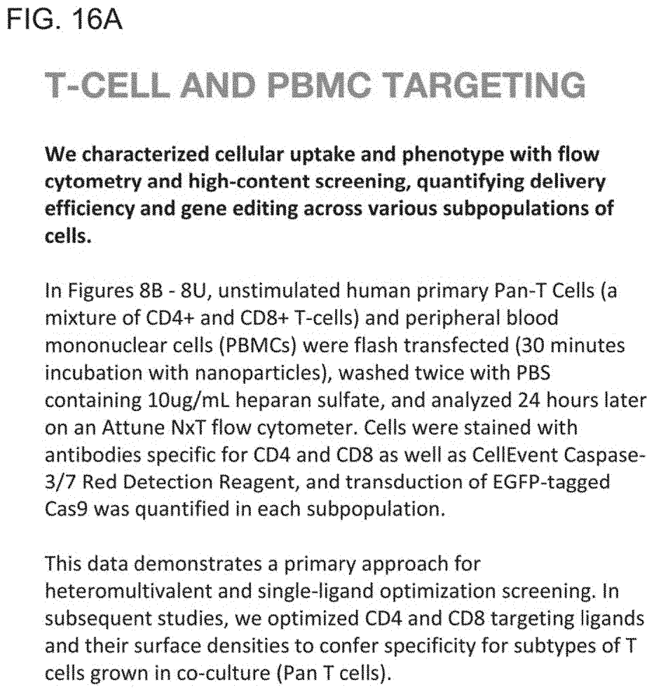

FIG. 22C depicts the robotic fluid handling associated with an iPad app for performing cellular media changes and washes, as well as cellular transfections via an Opentrons robot. These nanoparticles are either synthesized via separate robotic synthesis (via Andrew Robot and formulator app), as in FIGS. 13C-13J, or through a combination of microfluidic synthesis techniques and/or bulk robotic assembly techniques as detailed in FIGS. 15B1-15G3. In this figure, nanoparticle previously synthesized via the Formulator App (clear 96-well deep well plate) are transferred to 20,000 human primary Pan T cells per well (96-well clear bottom black plate) prior to subsequent imaging, flow cytometry, genomics, and nanoparticle characterization. Polypeptides forming nanoparticles in the clear 96-well plate were synthesized via custom high-throughput peptide synthesis robot.

### DETAILED DESCRIPTION

Before the present methods and compositions are described, it is to be understood that this invention is not limited to the particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the nanoparticle” includes reference to one or more nanoparticles and equivalents thereof, known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### Methods and Compositions

As noted above, provided are methods and compositions for the heterologous expression of a payload (e.g., DNA, RNA, protein) of interest in a target cell (e.g., cancer cell). In some cases payload delivery results in expression of a secreted protein, e.g., an immune signal such as a cytokine (e.g., by a cancer cell in vivo). In some cases payload delivery results in expression of a plasma membrane-tethered affinity marker (e.g., by cancer cells in vivo—thus resulting in an induced immune response). In some cases payload delivery results in expression of a cytotoxic protein such as an apoptosis inducer (e.g., by a cancer cell in vivo). Payloads are delivered with a delivery vehicle and in some cases the delivery vehicle is a nanoparticle. In some cases a subject nanoparticle for delivering payloads such as those discussed above includes a targeting ligand for targeted delivery to a specific cell type/tissue type (e.g., a cancerous tissue/cell).

In some embodiments, payload delivery is “personalized” in the sense that the delivery vehicle and/or payload is designed based on patient-specific information—such embodiments are referred to herein as “personalized” or “diagnostically-responsive” methods. As such, in some cases a subject method involves diagnostically-responsive payload delivery (i.e., personalized payload delivery)—in such cases the delivery vehicle and/or the payload can be considered “personalized.” In some embodiments, the “personalized” or “diagnostically-responsive” designation is due to the fact that one or more targeting ligands were identified/selected/designed/screened—for based on an individual's molecular data (e.g., sequencing data, array data, expression data, proteomics data, and the like). In some embodiments, the “personalized” or “diagnostically-responsive” designation is due to the fact that the payload was selected based on an individual's molecular data (e.g., sequencing data, array data, expression data, proteomics data, and the like).

Below is a general description of suitable “delivery vehicles” such as nanoparticles and their components, including an initial general description of payloads. This is followed by a description of ways in which such delivery vehicles and/or payloads can be ‘personalized’ in a diagnostically responsive way. Various payloads of interest (e.g., secreted proteins or nucleic acids encoding them, cytotoxic proteins or nucleic acids encoding them, and affinity markers or nucleic acids encoding them) are also described.

In some embodiments, one or more of the steps of the disclosed methods may be performed in a automated way—for example by a processor executing instructions, e.g., a non-transitory recording medium comprising instructions which, when executed by a processor of the system, cause the processor to perform any one or more of a variety of tasks, which can include but are not limited to: evaluating expression data, identifying one or more cell surface targets for targeting a cell, tissue, or organ of interest, generating a list of candidate targeting ligands (e.g., by evaluating crystal structures of the one or more cell surface targets to derive protein-ligand or protein-protein interaction information for the one or more cell surface targets), designing candidate targeting ligands, producing candidate targeting ligands (e.g., by actuating a robotic devise such as a liquid handling robot), producing a library of candidate delivery vehicles such as a library of nanoparticle formulations (e.g., by actuating a robotic devise such as a liquid handling robot), contacting surface targets (e.g., targets on the surface of cells) with candidate delivery vehicles such as candidate nanoparticle formulations, evaluating effectiveness of candidate targeting ligands and/or candidate delivery vehicles (e.g., via calculating measures of success based on a list of evaluation parameters), selecting the top-performing targeting ligands and/or delivery vehicle formulations, performing any of the above as part of a recursive screen (e.g., for targeting ligand and/or delivery vehicle optimization), and the like.

Delivery Vehicles

A delivery vehicle is a vehicle for delivering a payload (e.g., nucleic acid and/or protein payload) to a cell. Delivery vehicles can include, but are not limited to, non-viral vehicles, viral vehicles, nanoparticles (e.g., a nanoparticle that includes a targeting ligand and/or a core comprising an anionic polymer composition, a cationic polymer composition, and a cationic polypeptide composition), liposomes, micelles, water-oil-water emulsion particles, oil-water emulsion micellar particles, multilamellar water-oil-water emulsion particles, a targeting ligand (e.g., peptide targeting ligand) conjugated to a charged polymer polypeptide domain (where the targeting ligand provides for targeted binding to a cell surface protein, and the charged polymer polypeptide domain is condensed with a nucleic acid payload and/or is interacting electrostatically with a protein payload), a targeting ligand (e.g., peptide targeting ligand) conjugated to payload (where the targeting ligand provides for targeted binding to a cell surface protein). In some cases payloads are introduced into the cell as a deoxyribonucleoprotein complex or a ribo-deoxyribonucleoprotein complex.

In some cases, a delivery vehicle is a water-oil-water emulsion particle. In some cases, a delivery vehicle is an oil-water emulsion micellar particle. In some cases, a delivery vehicle is a multilamellar water-oil-water emulsion particle. In some cases, a delivery vehicle is a multilayered particle. In some cases, a delivery vehicle is a DNA origami nanobot. For any of the above a payload (nucleic acid and/or protein) can be inside of the particle, either covalently, bound as nucleic acid complementary pairs, or within a water phase of a particle. In some cases a delivery vehicle includes a targeting ligand, e.g., in some cases a targeting ligand (described in more detail elsewhere herein) coated upon a water-oil-water emulsion particle, upon an oil-water emulsion micellar particle, upon a multilamellar water-oil-water emulsion particle, upon a multilayered particle, or upon a DNA origami nanobot. In some cases a delivery vehicle has a solid core particle (e.g., metal particle core, quantum dot core, and the like)—in which case the payload can be conjugated to (covalently bound to) the core.

Payloads

Delivery vehicles (e.g., nanoparticles) of the disclosure include a payload (they are used to deliver a payload). A payload can be any compound one wishes to deliver to a cell. For example, in some cases a payload is a nucleic acid and/or protein. In some cases, a subject nanoparticle (e.g., a nanoparticle that includes a targeting ligand and/or a core comprising an anionic polymer composition, a cationic polymer composition, and a cationic polypeptide composition) is used to deliver a nucleic acid payload (e.g., a DNA and/or RNA). In some cases a subject nanoparticle (e.g., a nanoparticle that includes a targeting ligand and/or a core comprising an anionic polymer composition, a cationic polymer composition, and a cationic polypeptide composition) is used to deliver a protein payload. In some cases a subject nanoparticle (e.g., a nanoparticle that includes a targeting ligand and/or a core comprising an anionic polymer composition, a cationic polymer composition, and a cationic polypeptide composition) is used to deliver a payload of protein and nucleic acid, e.g., a ribonucleic acid protein complex (an RNP). A payload can be any desired compound. For example, in some cases a payload is a small molecule drug (e.g., which can be delivered via liposomes, nanoparticles as described herein such as PLGA particles, via direct conjugation to a targeting ligand, etc). For example in some cases a targeting ligand is used to direct the delivery of a small molecule drug via any convenient delivery vehicle (e.g., any of the delivery vehicles described herein can be used to deliver a small molecule drug payload).

A nucleic acid payload can be any nucleic acid of interest, e.g., the nucleic acid payload can be linear or circular, and can be a plasmid, a viral genome, an RNA (e.g., a coding RNA such as an mRNA or a non-coding RNA such as a guide RNA, a short interfering RNA (siRNA), a short hairpin RNA (shRNA), a microRNA (miRNA), and the like), a DNA, etc. In some cases, the nucleic payload is an RNAi agent (e.g., an shRNA, an siRNA, a miRNA, etc.) or a DNA template encoding an RNAi agent. In some cases, the nucleic acid payload is an siRNA molecule (e.g., one that targets an mRNA, one that targets a miRNA). In some cases, the nucleic acid payload is an LNA molecule (e.g., one that targets a miRNA). In some cases, the nucleic acid payload is a miRNA. In some cases the nucleic acid payload includes an mRNA that encodes a protein of interest (e.g., one or more reprograming and/or transdifferentiation factors such as Oct4, Sox2, Klf4, c-Myc, Nanog, and Lin28, e.g., alone or in any desired combination such as (i) Oct4, Sox2, Klf4, and c-Myc; (ii) Oct4, Sox2, Nanog, and Lin28; and the like; a gene editing endonuclease; a therapeutic protein; and the like). In some cases the nucleic acid payload includes a non-coding RNA (e.g., an RNAi agent, a CRISPR/Cas guide RNA, etc.) and/or a DNA molecule encoding the non-coding RNA. In some embodiments a nucleic acid payload includes a nucleic acid (DNA and/or mRNA) that encodes IL2Ra and IL12R₇ (e.g., to modulate the behavior or survival of a target cell), and in some cases the payload is released intracellularly from a subject nanoparticle over the course of from 7-90 days (e.g., from 7-80, 7-60, 7-50, 7-40, 7-35, or 7-30 days). In some cases the nucleic acid payload includes a self-replicating RNA.

In some embodiments a nucleic acid payload includes a nucleic acid (DNA and/or mRNA) that encodes BCL-XL (e.g., to prevent apoptosis of a target cell due to engagement of Fas or TNFα receptors). In some embodiments a nucleic acid payload includes a nucleic acid (DNA and/or mRNA) that encodes Foxp3 (e.g., to promote an immune effector phenotype in targeted T-cells). In some embodiments a nucleic acid payload includes a nucleic acid (DNA and/or mRNA) that encodes SCF. In some embodiments a nucleic acid payload includes a nucleic acid (DNA and/or mRNA) that encodes HoxB4. In some embodiments a nucleic acid payload includes a nucleic acid (DNA and/or mRNA) that encodes SIRT6. In some embodiments a nucleic acid payload includes a nucleic acid molecule (e.g., an siRNA, an LNA, etc.) that targets (reduces expression of) a microRNA such as miR-155 (see, e.g., MiR Base accession: MI0000681 and MI0000177). In some embodiments a nucleic acid payload includes an siRNA that targets ku70 and/or an siRNA that targets ku80.

The term “nucleic acid payload” encompasses modified nucleic acids. Likewise, the terms “RNAi agent” and “siRNA” encompass modified nucleic acids. For example, the nucleic acid molecule can be a mimetic, can include a modified sugar backbone, one or more modified internucleoside linkages (e.g., one or more phosphorothioate and/or heteroatom internucleoside linkages), one or more modified bases, and the like. In some embodiments, a subject payload includes triplex-forming peptide nucleic acids (PNAs) (see, e.g., McNeer et al., Gene Ther. 2013 June; 20(6):658-69). Thus, in some cases a subject core includes PNAs. In some cases a subject core includes PNAs and DNAs.

A subject nucleic acid payload (e.g., an siRNA) can have a morpholino backbone structure. In some case, a subject nucleic acid payload (e.g., an siRNA) can have one or more locked nucleic acids (LNAs). Suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. Suitable base modifications include synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

In some cases, a nucleic acid payload can include a conjugate moiety (e.g., one that enhances the activity, stability, cellular distribution or cellular uptake of the nucleic acid payload). These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Any convenient polynucleotide can be used as a subject nucleic acid payload. Examples include but are not limited to: species of RNA and DNA including mRNA, m1A modified mRNA (monomethylation at position 1 of Adenosine), siRNA, miRNA, aptamers, shRNA, AAV-derived nucleic acids and scaffolds, morpholino RNA, peptoid and peptide nucleic acids, cDNA, DNA origami, DNA and RNA with synthetic nucleotides, DNA and RNA with predefined secondary structures, multimers and oligomers of the aforementioned, and payloads whose sequence may encode other products such as any protein or polypeptide whose expression is desired.

In some cases a payload of a subject delivery vehicle (e.g., nanoparticle) includes a protein. Examples of protein payloads include, but are not limited to: programmable gene editing proteins (e.g., transcription activator-like (TAL) effectors (TALEs), TALE nucleases (TALENs), zinc-finger proteins (ZFPs), zinc-finger nucleases (ZFNs), DNA-guided polypeptides such as Natronobacterium gregoryi Argonaute (NgAgo), CRISPR/Cas RNA-guided polypeptide (Class 2 CRISPR/Cas effector protein) (e.g., Cas9, CasX, CasY, Cpf1, Cas13, MAD7, and the like); transposons (e.g., a Class I or Class II transposon—e.g., piggybac, sleeping beauty, Tc1/mariner, To12, PIF/harbinger, hAT, mutator, merlin, transib, helitron, maverick, frog prince, minos, Himarl and the like); meganucleases (e.g., I-SceI, I-CeuI, I-CreI, I-DmoI, I-ChuI, I-DirI, I-FlmuI, I-FlmuII, I-Anil, I-SceIV, I-CsmI, I-PanI, I-PanII, I-PanMI, I-SceII, I-PpoI, I-SceIII, I-LtrI, I-GpiI, I-GZeI, I-OnuI, I-HjeMI, I-Msol, I-Teel, I-TevII, I-TevIII, PI-MleI, PI-MtuI, PI-PspI, PI-Tli I, PI-Tli II, PI-SceV, and the like); megaTALs (see, e.g., Boissel et al., Nucleic Acids Res. 2014 February; 42(4): 2591-2601); SCF; BCL-XL; Foxp3; HoxB4; and SiRT6. For any of the above proteins, a payload of a subject delivery vehicle (e.g., nanoparticle) can include a nucleic acid (DNA and/or mRNA) encoding the protein, and/or can include the actual protein.

Gene Editing Tools (as Payloads)

In some cases, a nucleic acid payload includes or encodes a gene editing tool (i.e., a component of a gene editing system, e.g., a site specific gene editing system such as a programmable gene editing system). For example, a nucleic acid payload can include one or more of: (i) a CRISPR/Cas guide RNA, (ii) a DNA encoding a CRISPR/Cas guide RNA, (iii) a DNA and/or RNA encoding a programmable gene editing protein such as a zinc finger protein (ZFP) (e.g., a zinc finger nuclease—ZFN), a transcription activator-like effector (TALE) protein (e.g., fused to a nuclease—TALEN), a DNA-guided polypeptide such as Natronobacterium gregoryi Argonaute (NgAgo), and/or a CRISPR/Cas RNA-guided polypeptide (Class 2 CRISPR/Cas effector protein) (e.g., Cas9, CasX, CasY, Cpf1, Cas13, MAD7, and the like); (iv) a DNA donor template; (v) a nucleic acid molecule (DNA, RNA) encoding a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb 1 integrase, R4 integrase, lambda integrase, HK022 integrase, HP1 integrase, and the like); (vi) a DNA encoding a resolvase and/or invertase (e.g., Gin, Hin, γδ3, Tn3, Sin, Beta, and the like); and (vii) a transposon and/or a DNA derived from a transposon (e.g., bacterial transposons such as Tn3, Tn5, Tn7, Tn9, Tn10, Tn903, Tn1681, and the like; eukaryotic transposons such as Tc1/mariner super family transposons, PiggyBac superfamily transposons, hAT superfamily transposons, PiggyBac, Sleeping Beauty, Frog Prince, Minos, Himarl, and the like). In some cases a subject delivery vehicle (e.g., nanoparticle) is used to deliver a protein payload, e.g., a gene editing protein such as a ZFP (e.g., ZFN), a TALE (e.g., TALEN), a DNA-guided polypeptide such as Natronobacterium gregoryi Argonaute (NgAgo), a CRISPR/Cas RNA-guided polypeptide (Class 2 CRISPR/Cas effector protein) (e.g., Cas9, CasX, CasY, Cpf1, Cas13, MAD7, and the like), a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb 1 integrase, R4 integrase, lambda integrase, HK022 integrase, HP1 integrase, and the like), a resolvase/invertase (e.g., Gin, Hin, γδ3, Tn3, Sin, Beta, and the like); and/or a transposase (e.g., a transposase related to transposons such as bacterial transposons such as Tn3, Tn5, Tn7, Tn9, Tn10, Tn903, Tn1681, and the like; or eukaryotic transposons such as Tc1/mariner super family transposons, PiggyBac superfamily transposons, hAT superfamily transposons, PiggyBac, Sleeping Beauty, Frog Prince, Minos, Himarl, and the like). In some cases, the delivery vehicle (e.g., nanoparticle) is used to deliver a nucleic acid payload and a protein payload, and in some such cases the payload includes a ribonucleoprotein complex (RNP).

Depending on the nature of the system and the desired outcome, a gene editing system (e.g. a site specific gene editing system such as a a programmable gene editing system) can include a single component (e.g., a ZFP, a ZFN, a TALE, a TALEN, a site-specific recombinase, a resolvase/integrase, a transpose, a transposon, and the like) or can include multiple components. In some cases a gene editing system includes at least two components. For example, in some cases a gene editing system (e.g. a programmable gene editing system) includes (i) a donor template nucleic acid; and (ii) a gene editing protein (e.g., a programmable gene editing protein such as a ZFP, a ZFN, a TALE, a TALEN, a DNA-guided polypeptide such as Natronobacterium gregoryi Argonaute (NgAgo), a CRISPR/Cas RNA-guided polypeptide (Class 2 CRISPR/Cas effector protein) (e.g., Cas9, CasX, CasY, Cpf1, Cas13, MAD7, and the like), or a nucleic acid molecule encoding the gene editing protein (e.g., DNA or RNA such as a plasmid or mRNA). As another example, in some cases a gene editing system (e.g. a programmable gene editing system) includes (i) a CRISPR/Cas guide RNA, or a DNA encoding the CRISPR/Cas guide RNA; and (ii) a CRISPR/Cas RNA-guided polypeptide (Class 2 CRISPR/Cas effector protein) (e.g., Cas9, CasX, CasY, Cpf1, Cas13, MAD7, and the like), or a nucleic acid molecule encoding the RNA-guided polypeptide (e.g., DNA or RNA such as a plasmid or mRNA). As another example, in some cases a gene editing system (e.g. a programmable gene editing system) includes (i) an NgAgo-like guide DNA; and (ii) a DNA-guided polypeptide (e.g., NgAgo), or a nucleic acid molecule encoding the DNA-guided polypeptide (e.g., DNA or RNA such as a plasmid or mRNA). In some cases a gene editing system (e.g. a programmable gene editing system) includes at least three components: (i) a donor DNA template; (ii) a CRISPR/Cas guide RNA, or a DNA encoding the CRISPR/Cas guide RNA; and (iii) a CRISPR/Cas RNA-guided polypeptide (Class 2 CRISPR/Cas effector protein) (e.g., Cas9, CasX, CasY, Cpf1, Cas13, MAD7, and the like), or a nucleic acid molecule encoding the RNA-guided polypeptide (e.g., DNA or RNA such as a plasmid or mRNA). In some cases a gene editing system (e.g. a programmable gene editing system) includes at least three components: (i) a donor DNA template; (ii) an NgAgo-like guide DNA, or a DNA encoding the NgAgo-like guide DNA; and (iii) a DNA-guided polypeptide (e.g., NgAgo), or a nucleic acid molecule encoding the DNA-guided polypeptide (e.g., DNA or RNA such as a plasmid or mRNA).

In some embodiments, a subject delivery vehicle (e.g., nanoparticle) is used to deliver a gene editing tool. In other words in some cases the payload includes one or more gene editing tools. The term “gene editing tool” is used herein to refer to one or more components of a gene editing system. Thus, in some cases the payload includes a gene editing system and in some cases the payload includes one or more components of a gene editing system (i.e., one or more gene editing tools). For example, a target cell might already include one of the components of a gene editing system and the user need only add the remaining components. In such a case the payload of a subject delivery vehicle (e.g., nanoparticle) does not necessarily include all of the components of a given gene editing system. As such, in some cases a payload includes one or more gene editing tools.

As an illustrative example, a target cell might already include a gene editing protein (e.g., a ZFP, a TALE, a DNA-guided polypeptide (e.g., NgAgo), a CRISPR/Cas RNA-guided polypeptide (Class 2 CRISPR/Cas effector protein) (e.g., Cas9, CasX, CasY, Cpf1, Cas13, MAD7, and the like, a site-specific recombinase such as Cre recombinase, Dre recombinase, Flp recombinase, KD recombinase, B2 recombinase, B3 recombinase, R recombinase, Hin recombinase, Tre recombinase, PhiC31 integrase, Bxb 1 integrase, R4 integrase, lambda integrase, HK022 integrase, HP1 integrase, and the like, a resolvase/invertase such as Gin, Hin, γδ3, Tn3, Sin, Beta, and the like, a transposase, etc.) and/or a DNA or RNA encoding the protein, and therefore the payload can include one or more of: (i) a donor template; and (ii) a CRISPR/Cas guide RNA, or a DNA encoding the CRISPR/Cas guide RNA; or an NgAgo-like guide DNA. Likewise, the target cell may already include a CRISPR/Cas guide RNA and/or a DNA encoding the guide RNA or an NgAgo-like guide DNA, and the payload can include one or more of: (i) a donor template; and (ii) a CRISPR/Cas RNA-guided polypeptide (Class 2 CRISPR/Cas effector protein) (e.g., Cas9, CasX, CasY, Cpf1, Cas13, MAD7, and the like), or a nucleic acid molecule encoding the RNA-guided polypeptide (e.g., DNA or RNA such as a plasmid or mRNA); or a DNA-guided polypeptide (e.g., NgAgo), or a nucleic acid molecule encoding the DNA-guided polypeptide.

As would be understood by one of ordinary skill in the art, a gene editing system need not be a system that ‘edits’ a nucleic acid. For example, it is well recognized that a gene editing system can be used to modify target nucleic acids (e.g., DNA and/or RNA) in a variety of ways without creating a double strand break (DSB) in the target DNA. For example, in some cases a double stranded target DNA is nicked (one strand is cleaved), and in some cases (e.g., in some cases where the gene editing protein is devoid of nuclease activity, e.g., a CRISPR/Cas RNA-guided polypeptide may harbor mutations in the catalytic nuclease domains), the target nucleic acid is not cleaved at all. For example, in some cases a CRISPR/Cas protein (e.g., Cas9, CasX, CasY, Cpf1) with or without nuclease activity, is fused to a heterologous protein domain. The heterologous protein domain can provide an activity to the fusion protein such as (i) a DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity), (ii) a transcription modulation activity (e.g., fusion to a transcriptional repressor or activator), or (iii) an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). As such, a gene editing system can be used in applications that modify a target nucleic acid in way that do not cleave the target nucleic acid, and can also be used in applications that modulate transcription from a target DNA.

For additional information related to programmable gene editing tools (e.g., CRISPR/Cas RNa-guided proteins such as Cas9, CasX, CasY, Cpf1, Cas13, MAD7, and the like, Zinc finger proteins such as Zinc finger nucleases, TALE proteins such as TALENs, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. al., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 Jul. 34(7):768-73; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

In some cases an inserted nucleotide sequence (e.g., of a donor DNA) encodes a receptor whereby the target that is targeted (bound) by the receptor is specific to an individual's disease (e.g., cancer/tumor). In some cases an inserted nucleotide sequence (e.g., of a donor DNA) encodes a heteromultivalent receptor, whereby the combination of targets that are targeted by the heteromultivalent receptor are specific to an individual's disease (e.g., cancer/tumor). As one illustrative example, an individual's cancer (e.g., tumor, e.g., via biopsy) can be sequenced (nucleic acid sequence, proteomics, metabolomics etc.) to identify antigens of diseased cells that can be targets (such as antigens that are overexpressed by or are unique to a tumor relative to control cells of the individual), and a nucleotide sequence encoding a receptor (e.g., heteromultivalent receptor) that binds to one or more of those targets (e.g., 2 or more, 3 or more, 5 or more, 10 or more, 15 or more, or about 20 of those targets) can be inserted into an immune cell (e.g., an NK cell, a B-Cell, a T-Cell, e.g., using a CAR or TCR) so that the immune cell specifically targets the individual's disease cells (e.g., tumor cells). As such, an inserted nucleotide sequence (e.g., of a donor DNA) can be designed to be diagnostically responsive—in the sense that the encoded receptor(s) (e.g., heteromultivalent receptor(s)) can be designed after receiving unique insights related to a patient's proteomics, genomics or metabolomics (e.g., through sequencing etc.)—thus generating an avid and specific immune system response. In this way, immune cells (such as NK cells, B cell, T cells, and the like) can be genome edited to express receptors such as CAR and/or TCR proteins (e.g., heteromultivalent versions) that are designed to be effective against an individual's own disease (e.g., cancer). In some cases, regulatory T cells can be given similar avidity for tissues affected by autoimmunity following diagnostically-responsive medicine. In some cases, antigen presenting cells (such as Macrophages, Dendritic cells, B cells, and the like) can be edited to more effectively present or recognize antigens based on a diagnostically-responsive process.

In some cases the nucleotide sequence, of a donor DNA that is inserted into a cell's genome includes a protein-coding nucleotide sequence that does not have introns. In some cases the nucleotide sequence that does not have introns encodes all or a portion of a TCR protein.

In some embodiments more than one delivery vehicle is introduced into a target cell. For example, in some cases a subject method includes introducing a first and a second of said delivery vehicles into the cell, where a nucleotide sequence of a donor DNA of the first delivery vehicle, that is inserted into the cell's genome, encodes a T cell receptor (TCR) Alpha or Delta subunit, and the nucleotide sequence of the donor DNA of the second delivery vehicle, that is inserted into the cell's genome, encodes a TCR Beta or Gamma subunit. In some cases a subject method includes introducing a first and a second of said delivery vehicles into the cell, where the nucleotide sequence of the donor DNA of the first delivery vehicle, that is inserted into the cell's genome, encodes a T cell receptor (TCR) Alpha or Delta subunit constant region, and the nucleotide sequence of the donor DNA of the second delivery vehicle, that is inserted into the cell's genome, encodes a TCR Beta or Gamma subunit constant region.

In some cases a subject method includes introducing a first and a second of said delivery vehicles into the cell, wherein the nucleotide sequence of a donor DNA of the first delivery vehicle is inserted within a nucleotide sequence that functions as a T cell receptor (TCR) Alpha or Delta subunit promoter, and the nucleotide sequence of a donor DNA of the second delivery vehicle is inserted within a nucleotide sequence that functions as a TCR Beta or Gamma subunit promoter. For more information related to TCR proteins and CDRs, see, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21. In some cases, a 147 bp TCRbeta promoter can drive high cell-specific gene expression in T cells, and may include the sequence:

Agtcacccaagtgtggtctaatataaatcctgtgttcctgaggtcatgcagattgagagaggaagtgatgtcactgtgggaacttccgtgtaagga cggggcgtccctcctcctctgctcctgctcacagtgatcctgatctggtaa (SEQ ID NO: xx)

In some cases a subject method includes introducing a first and a second of said delivery vehicles into the cell, where the nucleotide sequence of a donor DNA of the first delivery vehicle, that is inserted into the cell's genome, encodes a T cell receptor (TCR) Alpha or Gamma subunit, and the nucleotide sequence of a donor DNA of the second delivery vehicle, that is inserted into the cell's genome, encodes a TCR Beta or Delta subunit. In some cases a subject method includes introducing a first and a second of said delivery vehicles into the cell, where the nucleotide sequence of the donor DNA of the first delivery vehicle, that is inserted into the cell's genome, encodes a T cell receptor (TCR) Alpha or Delta subunit constant region, and the nucleotide sequence of the donor DNA of the second delivery vehicle, that is inserted into the cell's genome, encodes a TCR Beta or Gamma subunit constant region. In some cases a subject method includes introducing a first and a second of said delivery vehicles into the cell, wherein the nucleotide sequence of the donor DNA of the first delivery vehicle is inserted within a nucleotide sequence that functions as a T cell receptor (TCR) Alpha or Gamma subunit promoter, and the nucleotide sequence of the donor DNA of the second delivery vehicle is inserted within a nucleotide sequence that functions as a TCR Beta or Delta subunit promoter. For more information related to TCR proteins and CDRs, see, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21.

Payloads for Co-Delivery

In some embodiments, more than one payload is delivered as part of the same package (e.g., nanoparticle), e.g., in some cases different payloads are part of different cores. One advantage of delivering multiple payloads as part of the same package (e.g., nanoparticle) is that the efficiency of each payload is not diluted. As an illustrative example, if payload A and payload B are delivered in two separate packages (package A and package B, respectively), then the efficiencies are multiplicative, e.g., if package A and package B each have a 1% transfection efficiency, the chance of delivering payload A and payload B to the same cell is 0.01% (1%×1%). However, if payload A and payload B are both delivered as part of the same package (e.g., part of the same nanoparticle—package A), then the chance of delivering payload A and payload B to the same cell is 1%, a 100-fold improvement over 0.01%.

Likewise, in a scenario where package A and package B each have a 0.1% transfection efficiency, the chance of delivering payload A and payload B to the same cell is 0.0001% (0.1%×0.1%). However, if payload A and payload B are both delivered as part of the same package (e.g., part of the same nanoparticle—package A) in this scenario, then the chance of delivering payload A and payload B to the same cell is 0.1%, a 1000-fold improvement over 0.0001%.

As such, in some embodiments, one or more gene editing tools (e.g., as described above) is delivered in combination with (e.g., as part of the same nanoparticle) a protein (and/or a DNA or mRNA encoding same) and/or a non-coding RNA that increases genomic editing efficiency. In some cases, one or more gene editing tools (e.g., as described above) is delivered in combination with (e.g., as part of the same nanoparticle) a protein (and/or a DNA or mRNA encoding same) and/or a non-coding RNA that controls cell division and/or differentiation. In some cases, one or more gene editing tools (e.g., as described above) is delivered in combination with (e.g., as part of the same nanoparticle) a protein (and/or a DNA or mRNA encoding same) and/or a non-coding RNA that biases the cell DNA repair machinery toward non-homologous end joining (NHEJ) or homology directed repair (HDR).

As non-limiting examples of the above, in some embodiments one or more gene editing tools can be delivered in combination with one or more of: SCF (and/or a DNA or mRNA encoding SCF), HoxB4 (and/or a DNA or mRNA encoding HoxB4), BCL-XL (and/or a DNA or mRNA encoding BCL-XL), SIRT6 (and/or a DNA or mRNA encoding SIRT6), a nucleic acid molecule (e.g., an siRNA and/or an LNA) that suppresses miR-155, a nucleic acid molecule (e.g., an siRNA, an shRNA, a microRNA) that reduces ku70 expression, and a nucleic acid molecule (e.g., an siRNA, an shRNA, a microRNA) that reduces ku80 expression.

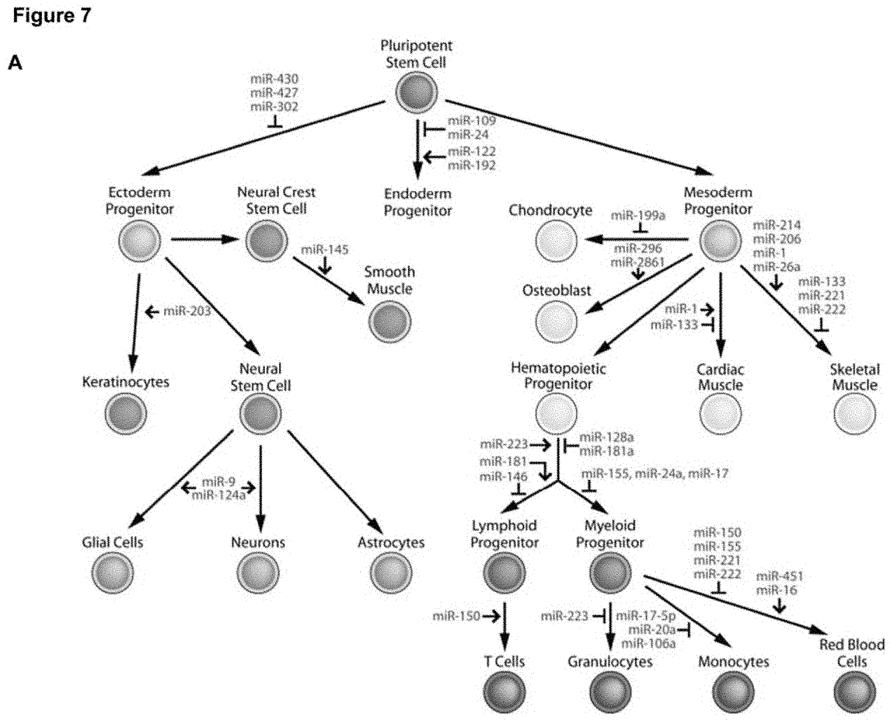

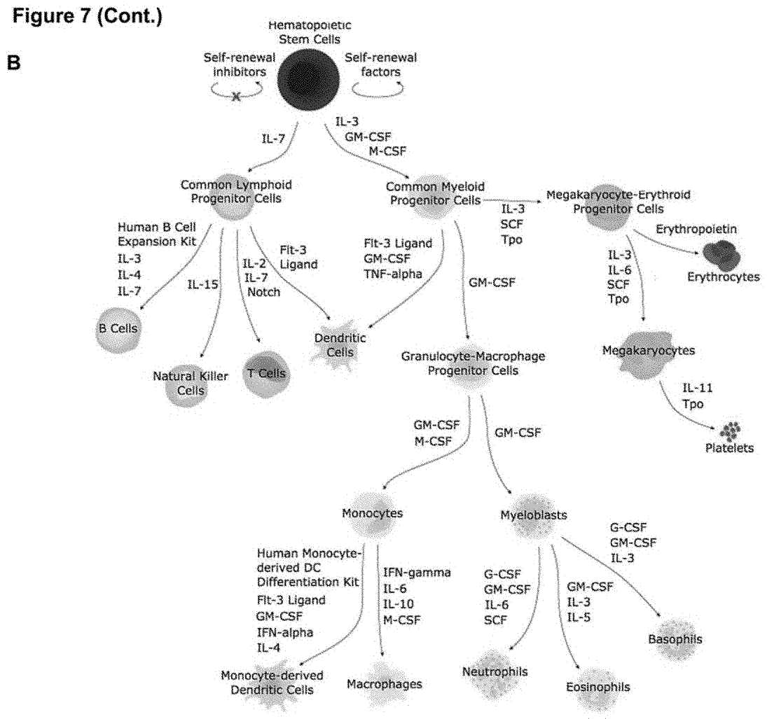

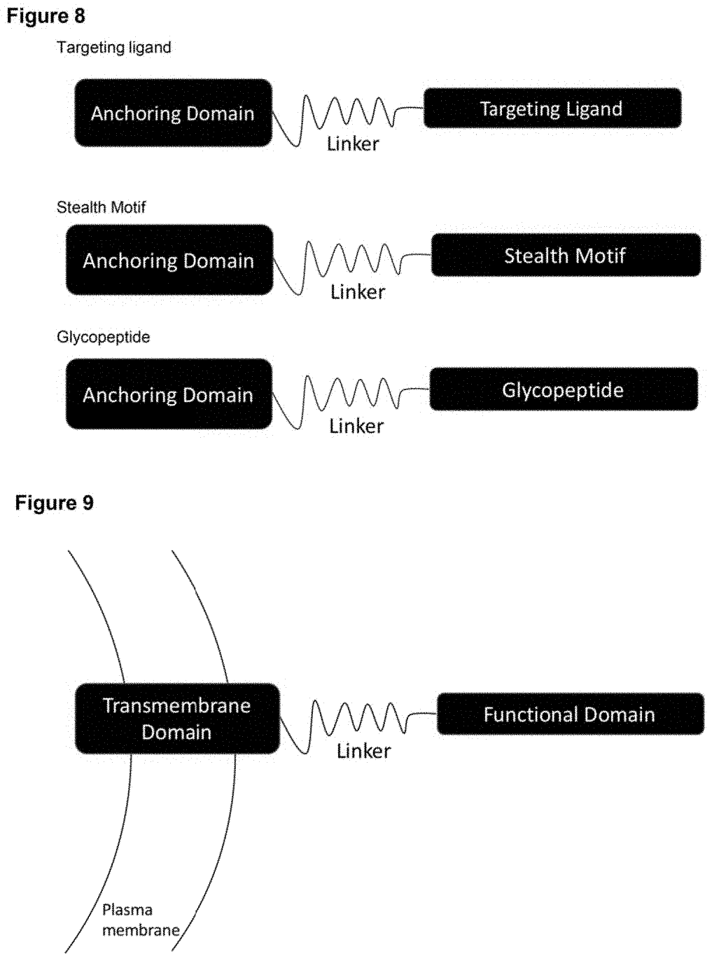

For examples of microRNAs that can be delivered in combination with a gene editing tool, see FIG. 7A. For example, the following microRNAs can be used for the following purposes: for blocking differentiation of a pluripotent stem cell toward ectoderm lineage: miR-430/427/302 (see, e.g., MiR Base accession: MI0000738, MI0000772, MI0000773, MI0000774, MI0006417, MI0006418, MI0000402, MI0003716, MI0003717, and MI0003718); for blocking differentiation of a pluripotent stem cell toward endoderm lineage: miR-109 and/or miR-24 (see, e.g., MiR Base accession: MI0000080, MI0000081, MI0000231, and MI0000572); for driving differentiation of a pluripotent stem cell toward endoderm lineage: miR-122 (see, e.g., MiR Base accession: MI0000442 and MI0000256) and/or miR-192 (see, e.g., MiR Base accession: MI0000234 and MI0000551); for driving differentiation of an ectoderm progenitor cell toward a keratinocyte fate: miR-203 (see, e.g., MiR Base accession: MI0000283, MI0017343, and MI0000246); for driving differentiation of a neural crest stem cell toward a smooth muscle fate: miR-145 (see, e.g., MiR Base accession: MI0000461, MI0000169, and MI0021890); for driving differentiation of a neural stem cell toward a glial cell fate and/or toward a neuron fate: miR-9 (see, e.g., MiR Base accession: MI0000466, MI0000467, MI0000468, MI0000157, MI0000720, and MI0000721) and/or miR-124a (see, e.g., MiR Base accession: MI0000443, MI0000444, MI0000445, MI0000150, MI0000716, and MI0000717); for blocking differentiation of a mesoderm progenitor cell toward a chondrocyte fate: miR-199a (see, e.g., MiR Base accession: MI0000242, MI0000281, MI0000241, and MI0000713); for driving differentiation of a mesoderm progenitor cell toward an osteoblast fate: miR-296 (see, e.g., MiR Base accession: MI0000747 and MI0000394) and/or miR-2861 (see, e.g., MiR Base accession: MI0013006 and MI0013007); for driving differentiation of a mesoderm progenitor cell toward a cardiac muscle fate: miR-1 (see, e.g., MiR Base accession: MI0000437, MI0000651, MI0000139, MI0000652, MI0006283); for blocking differentiation of a mesoderm progenitor cell toward a cardiac muscle fate: miR-133 (see, e.g., MiR Base accession: MI0000450, MI0000451, MI0000822, MI0000159, MI0000820, MI0000821, and MI0021863); for driving differentiation of a mesoderm progenitor cell toward a skeletal muscle fate: miR-214 (see, e.g., MiR Base accession: MI0000290 and MI0000698), miR-206 (see, e.g., MiR Base accession: MI0000490 and MI0000249), miR-1 and/or miR-26a (see, e.g., MiR Base accession: MI0000083, MI0000750, MI0000573, and MI0000706); for blocking differentiation of a mesoderm progenitor cell toward a skeletal muscle fate: miR-133 (see, e.g., MiR Base accession: MI0000450, MI0000451, MI0000822, MI0000159, MI0000820, MI0000821, and MI0021863), miR-221 (see, e.g., MiR Base accession: MI0000298 and MI0000709), and/or miR-222 (see, e.g., MiR Base accession: MI0000299 and MI0000710); for driving differentiation of a hematopoietic progenitor cell toward differentiation: miR-223 (see, e.g., MiR Base accession: MI0000300 and MI0000703); for blocking differentiation of a hematopoietic progenitor cell toward differentiation: miR-128a (see, e.g., MiR Base accession: MI0000447 and MI0000155) and/or miR-181a (see, e.g., MiR Base accession: MI0000269, MI0000289, MI0000223, and MI0000697); for driving differentiation of a hematopoietic progenitor cell toward a lymphoid progenitor cell: miR-181 (see, e.g., MiR Base accession: MI0000269, MI0000270, MI0000271, MI0000289, MI0000683, MI0003139, MI0000223, MI0000723, MI0000697, MI0000724, MI0000823, and MI0005450); for blocking differentiation of a hematopoietic progenitor cell toward a lymphoid progenitor cell: miR-146 (see, e.g., MiR Base accession: MI0000477, MI0003129, MI0003782, MI0000170, and MI0004665); for blocking differentiation of a hematopoietic progenitor cell toward a myeloid progenitor cell: miR-155, miR-24a, and/or miR-17 (see, e.g., MiR Base accession: MI0000071 and MI0000687); for driving differentiation of a lymphoid progenitor cell toward a T cell fate: miR-150 (see, e.g., MiR Base accession: MI0000479 and MI0000172); for blocking differentiation of a myeloid progenitor cell toward a granulocyte fate: miR-223 (see, e.g., MiR Base accession: MI0000300 and MI0000703); for blocking differentiation of a myeloid progenitor cell toward a monocyte fate: miR-17-5p (see, e.g., MiR Base accession: MIMAT0000070 and MIMAT0000649), miR-20a (see, e.g., MiR Base accession: MI0000076 and MI0000568), and/or miR-106a (see, e.g., MiR Base accession: MI0000113 and MI0000406); for blocking differentiation of a myeloid progenitor cell toward a red blood cell fate: miR-150 (see, e.g., MiR Base accession: MI0000479 and MI0000172), miR-155, miR-221 (see, e.g., MiR Base accession: MI0000298 and MI0000709), and/or miR-222 (see, e.g., MiR Base accession: MI0000299 and MI0000710); and for driving differentiation of a myeloid progenitor cell toward a red blood cell fate: miR-451 (see, e.g., MiR Base accession: MI0001729, MI0017360, MI0001730, and MI0021960) and/or miR-16 (see, e.g., MiR Base accession: MI0000070, MI0000115, MI0000565, and MI0000566).

For examples of signaling proteins (e.g., extracellular signaling proteins) that can be delivered (e.g., as protein or as DNA or RNA encoding the protein) in combination with a gene editing tool, see FIG. 7B. The same proteins can be used as part of the outer shell of a subject nanoparticle in a similar manner as a targeting ligand, e.g., for the purpose of biasing differentiation in target cells that receive the nanoparticle. For example, the following signaling proteins (e.g., extracellular signaling proteins) can be used for the following purposes: for driving differentiation of a hematopoietic stem cell toward a common lymphoid progenitor cell lineage: IL-7 (see, e.g., NCBI Gene ID 3574); for driving differentiation of a hematopoietic stem cell toward a common myeloid progenitor cell lineage: IL-3 (see, e.g., NCBI Gene ID 3562), GM-CSF (see, e.g., NCBI Gene ID 1437), and/or M-CSF (see, e.g., NCBI Gene ID 1435); for driving differentiation of a common lymphoid progenitor cell toward a B-cell fate: IL-3, IL-4 (see, e.g., NCBI Gene ID: 3565), and/or IL-7; for driving differentiation of a common lymphoid progenitor cell toward a Natural Killer Cell fate: IL-15 (see, e.g., NCBI Gene ID 3600); for driving differentiation of a common lymphoid progenitor cell toward a T-cell fate: IL-2 (see, e.g., NCBI Gene ID 3558), IL-7, and/or Notch (see, e.g., NCBI Gene IDs 4851, 4853, 4854, 4855); for driving differentiation of a common lymphoid progenitor cell toward a dendritic cell fate: Flt-3 ligand (see, e.g., NCBI Gene ID 2323); for driving differentiation of a common myeloid progenitor cell toward a dendritic cell fate: Flt-3 ligand, GM-CSF, and/or TNF-alpha (see, e.g., NCBI Gene ID 7124); for driving differentiation of a common myeloid progenitor cell toward a granulocyte-macrophage progenitor cell lineage: GM-CSF; for driving differentiation of a common myeloid progenitor cell toward a megakaryocyte-erythroid progenitor cell lineage: IL-3, SCF (see, e.g., NCBI Gene ID 4254), and/or Tpo (see, e.g., NCBI Gene ID 7173); for driving differentiation of a megakaryocyte-erythroid progenitor cell toward a megakaryocyte fate: IL-3, IL-6 (see, e.g., NCBI Gene ID 3569), SCF, and/or Tpo; for driving differentiation of a megakaryocyte-erythroid progenitor cell toward a erythrocyte fate: erythropoietin (see, e.g., NCBI Gene ID 2056); for driving differentiation of a megakaryocyte toward a platelet fate: IL-11 (see, e.g., NCBI Gene ID 3589) and/or Tpo; for driving differentiation of a granulocyte-macrophage progenitor cell toward a monocyte lineage: GM-CSF and/or M-CSF; for driving differentiation of a granulocyte-macrophage progenitor cell toward a myeloblast lineage: GM-CSF; for driving differentiation of a monocyte toward a monocyte-derived dendritic cell fate: Flt-3 ligand, GM-CSF, IFN-alpha (see, e.g., NCBI Gene ID 3439), and/or IL-4; for driving differentiation of a monocyte toward a macrophage fate: IFN-gamma, IL-6, IL-10 (see, e.g., NCBI Gene ID 3586), and/or M-CSF; for driving differentiation of a myeloblast toward a neutrophil fate: G-CSF (see, e.g., NCBI Gene ID 1440), GM-CSF, IL-6, and/or SCF; for driving differentiation of a myeloblast toward a eosinophil fate: GM-CSF, IL-3, and/or IL-5 (see, e.g., NCBI Gene ID 3567); and for driving differentiation of a myeloblast toward a basophil fate: G-CSF, GM-CSF, and/or IL-3.

Examples of proteins that can be delivered (e.g., as protein and/or a nucleic acid such as DNA or RNA encoding the protein) in combination with a gene editing tool include but are not limited to: SOX17, HEX, OSKM (Oct4/Sox2/K1f4/c-myc), and/or bFGF (e.g., to drive differentiation toward hepatic stem cell lineage); HNF4a (e.g., to drive differentiation toward hepatocyte fate); Poly (I:C), BMP-4, bFGF, and/or 8-Br-cAMP (e.g., to drive differentiation toward endothelial stem cell/progenitor lineage); VEGF (e.g., to drive differentiation toward arterial endothelium fate); Sox-2, Brn4, Mytl1, Neurod2, Ascl1 (e.g., to drive differentiation toward neural stem cell/progenitor lineage); and BDNF, FCS, Forskolin, and/or SHH (e.g., to drive differentiation neuron, astrocyte, and/or oligodendrocyte fate).

Examples of signaling proteins (e.g., extracellular signaling proteins) that can be delivered (e.g., as protein and/or a nucleic acid such as DNA or RNA encoding the protein) in combination with a gene editing tool include but are not limited to: cytokines (e.g., IL-2 and/or IL-15, e.g., for activating CD8+ T-cells); ligands and or signaling proteins that modulate one or more of the Notch, Wnt, and/or Smad signaling pathways; SCF; stem cell differentiating factors (e.g. Sox2, Oct3/4, Nanog, Klf4, c-Myc, and the like); and temporary surface marker “tags” and/or fluorescent reporters for subsequent isolation/purification/concentration. For example, a fibroblast may be converted into a neural stem cell via delivery of Sox2, while it will turn into a cardiomyocyte in the presence of Oct3/4 and small molecule “epigenetic resetting factors.” In a patient with Huntington's disease or a CXCR4 mutation, these fibroblasts may respectively encode diseased phenotypic traits associated with neurons and cardiac cells. By delivering gene editing corrections and these factors in a single package, the risk of deleterious effects due to one or more, but not all of the factors/payloads being introduced can be significantly reduced.

Because the timing and/or location of payload release can be controlled (described in more detail elsewhere in this disclosure), the packaging of multiple payloads in the same package (e.g., same nanoparticle) does not preclude one from achieving different release times and/or locations for different payloads. For example the release of the above proteins (and/or a DNAs or mRNAs encoding same) and/or non-coding RNAs can be controlled separately from the release of the one or more gene editing tools that are part of the same package. For example, proteins and/or nucleic acids (e.g., DNAs, mRNAs, non-coding RNAs, miRNAs) that control cell proliferation and/or differentiation, or that control bias toward NHEJ or HDR, can be released earlier than the one or more gene editing tools or can be released later than the one or more gene editing tools. This can be achieved, e.g., by using more than one sheddable layer and/or by using more than one core (e.g., where one core has a different release profile than the other, e.g., uses a different D- to L-isomer ratio, uses a different ESP:ENP:EPP profile, and the like).

Applications include in vivo approaches wherein a cell death cue may be conditional upon a gene edit not being successful, and cell differentiation/proliferation/activation is tied to a tissue/organ-specific promoter and/or exogenous factor. A diseased cell receiving a gene edit may activate and proliferate, but due to the presence of another promoter-driven expression cassette (e.g. one tied to the absence of tumor suppressor such as p21 or p53), those cells will subsequently be eliminated. The cells expressing desired characteristics, on the other hand, may be triggered to further differentiate into the desired downstream lineages.

In some cases, a subject nucleic acid payload includes a morpholino backbone structure. In some case, a subject nucleic acid payload can have one or more locked nucleic acids (LNAs). Suitable sugar substituent groups include methoxy (—O—CH₃), aminopropoxy (—O CH₂ CH₂ CH₂NH₂), allyl (—CH₂—CH═CH₂), —O-allyl (—O— CH₂—CH═CH₂) and fluoro (F). 2′-sugar substituent groups may be in the arabino (up) position or ribo (down) position. Suitable base modifications include synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

In some cases, a nucleic acid payload can include a conjugate moiety (e.g., one that enhances the activity, stability, cellular distribution or cellular uptake of the nucleic acid payload). These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Any convenient polynucleotide can be used as a subject nucleic acid payload. Examples include but are not limited to: species of RNA and DNA including mRNA, m1A modified mRNA (monomethylation at position 1 of Adenosine), morpholino RNA, peptoid and peptide nucleic acids, cDNA, DNA origami, DNA and RNA with synthetic nucleotides, DNA and RNA with predefined secondary structures, and multimers and oligomers of the aforementioned.

Because the timing and/or location of payload release can be controlled (described in more detail elsewhere in this disclosure), the packaging of multiple payloads in the same package (e.g., same nanoparticle) does not preclude one from achieving different release times/rates and/or locations for different payloads. For example, the release of the above proteins (and/or a DNAs or mRNAs encoding same) and/or non-coding RNAs can be controlled separately from the release of the one or more gene editing tools that are part of the same package. For example, proteins and/or nucleic acids (e.g., DNAs, mRNAs, non-coding RNAs, miRNAs) that control cell proliferation and/or differentiation can be released earlier than the one or more gene editing tools or can be released later than the one or more gene editing tools. This can be achieved, e.g., by using more than one sheddable layer and/or by using more than one core (e.g., where one core has a different release profile than the other, e.g., uses a different D- to L-isomer ratio, uses a different ESP:ENP:EPP profile, and the like). In this way, a donor and nuclease may be released in a stepwise manner that allows for optimal editing and insertion efficiencies.

### Nanoparticles

Nanoparticles of the disclosure include a payload, which can be made of nucleic acid and/or protein. For example, in some cases a subject nanoparticle is used to deliver a nucleic acid payload (e.g., a DNA and/or RNA). The payloads function to influence cellular phenotype, or result in the expression of proteins to be secreted or presented on the cell surface. In some cases the core of the nanoparticle includes the payload(s). In some such cases a nanoparticle core can also include an anionic polymer composition, a cationic polymer composition, and a cationic polypeptide composition. In some cases the nanoparticle has a metallic core and the payload associates with (in some cases is conjugated to, e.g., the outside of) the core. In some embodiments, the payload is part of the nanoparticle core. Thus the core of a subject nanoparticle can include nucleic acid, DNA, RNA, and/or protein. Thus, in some cases a subject nanoparticle includes nucleic acid (DNA and/or RNA) and protein. In some cases a subject nanoparticle core includes a ribonucleoprotein (RNA and protein) complex. In some cases a subject nanoparticle core includes a deoxyribonucleoprotein (DNA and protein, e.g., donor DNA and ZFN, TALEN, or CRISPR/Cas effector protein) complex. In some cases a subject nanoparticle core includes a ribo-deoxyribonucleoprotein (RNA and DNA and protein, e.g., a guide RNA, a donor DNA and a CRISPR/Cas effector protein) complex. In some cases a subject nanoparticle core includes PNAs. In some cases a subject core includes PNAs and DNAs.

Nanoparticles as described herein are modular and can be tailored for various scenarios: for example, each component (e.g., payload, core, coat, targeting ligand, etc.) can be selected based on the desired outcome, e.g., as part of a set of degrees of freedom across the entire nanoparticle platform.

Nanoparticle Core

The core of a subject nanoparticle can include an anionic polymer composition (e.g., poly(glutamic acid)), a cationic polymer composition (e.g., poly(arginine), a cationic polypeptide composition (e.g., a histone tail peptide), and a payload (e.g., nucleic acid and/or protein payload). In some cases the core is generated by condensation of a cationic amino acid polymer and payload in the presence of an anionic amino acid polymer (and in some cases in the presence of a cationic polypeptide of a cationic polypeptide composition). In some embodiments, condensation of the components that make up the core can mediate increased transfection efficiency compared to conjugates of cationic polymers with a payload. Inclusion of an anionic polymer in a nanoparticle core may prolong the duration of intracellular residence of the nanoparticle and release of payload.

Other nanoparticle cores may include proteins as substrates, whereas a molecule such as Cas9 has its surface modified by subsequent electrostatic or covalent layers encoding cell-specific targeting, subcellular trafficking characteristics, or tethering together multiple payloads (e.g. Cas9 protein and RNP forms with DNA covalently attached).

For the cationic and anionic polymer compositions of the core, ratios of D-isomer polymers to L-isomer polymers can be controlled in order to control the timed release of payload, where increased ratio of D-isomer polymers to L-isomer polymers leads to increased stability (reduced payload release rate), which for example can enable longer lasting gene expression from a payload delivered by a subject nanoparticle. In some cases modifying the ratio of D-to-L isomer polypeptides within the nanoparticle core can cause gene expression profiles (e.g., expression of a protein encoded by a payload molecule) to be on the order of from 1-90 days (e.g. from 1-80, 1-70, 1-60, 1-50, 1-40, 1-30, 1-25, 1-20, 1-15, 1-10, 3-90, 3-80, 3-70, 3-60, 3-50, 3-40, 3-30, 3-25, 3-20, 3-15, 3-10, 5-90, 5-80, 5-70, 5-60, 5-50, 5-40, 5-30, 5-25, 5-20, 5-15, or 5-10 days). The control of payload release (e.g., when delivering a gene editing tool), can be particularly effective for performing genomic edits e.g., in some cases where homology-directed repair is desired.

In some embodiments, a nanoparticle includes a core and a sheddable layer encapsulating the core, where the core includes: (a) an anionic polymer composition; (b) a cationic polymer composition; (c) a cationic polypeptide composition; and (d) a nucleic acid and/or protein payload, where one of (a) and (b) includes a D-isomer polymer of an amino acid, and the other of (a) and (b) includes an L-isomer polymer of an amino acid, and where the ratio of the D-isomer polymer to the L-isomer polymer is in a range of from 10:1 to 1.5:1 (e.g., from 8:1 to 1.5:1, 6:1 to 1.5:1, 5:1 to 1.5:1, 4:1 to 1.5:1, 3:1 to 1.5:1, 2:1 to 1.5:1, 10:1 to 2:1; 8:1 to 2:1, 6:1 to 2:1, 5:1 to 2:1, 10:1 to 3:1; 8:1 to 3:1, 6:1 to 3:1, 5:1 to 3:1, 10:1 to 4:1; 4:1 to 2:1, 6:1 to 4:1, or 10:1 to 5:1), or from 1:1.5 to 1:10 (e.g., from 1:1.5 to 1:8, 1:1.5 to 1:6, 1:1.5 to 1:5, 1:1.5 to 1:4, 1:1.5 to 1:3, 1:1.5 to 1:2, 1:2 to 1:10, 1:2 to 1:8, 1:2 to 1:6, 1:2 to 1:5, 1:2 to 1:4, 1:2 to 1:3, 1:3 to 1:10, 1:3 to 1:8, 1:3 to 1:6, 1:3 to 1:5, 1:4 to 1:10, 1:4 to 1:8, 1:4 to 1:6, or 1:5 to 1:10). In some such cases, the ratio of the D-isomer polymer to the L-isomer polymer is not 1:1. In some such cases, the anionic polymer composition includes an anionic polymer selected from poly(D-glutamic acid) (PDEA) and poly(D-aspartic acid) (PDDA), where (optionally) the cationic polymer composition can include a cationic polymer selected from poly(L-arginine), poly(L-lysine), poly(L-histidine), poly(L-ornithine), and poly(L-citrulline). In some cases the cationic polymer composition comprises a cationic polymer selected from poly(D-arginine), poly(D-lysine), poly(D-histidine), poly(D-ornithine), and poly(D-citrulline), where (optionally) the anionic polymer composition can include an anionic polymer selected from poly(L-glutamic acid) (PLEA) and poly(L-aspartic acid) (PLDA).

In some embodiments, a nanoparticle includes a core and a sheddable layer encapsulating the core, where the core includes: (i) an anionic polymer composition; (ii) a cationic polymer composition; (iii) a cationic polypeptide composition; and (iv) a nucleic acid and/or protein payload, wherein (a) said anionic polymer composition includes polymers of D-isomers of an anionic amino acid and polymers of L-isomers of an anionic amino acid; and/or (b) said cationic polymer composition includes polymers of D-isomers of a cationic amino acid and polymers of L-isomers of a cationic amino acid. In some such cases, the anionic polymer composition comprises a first anionic polymer selected from poly(D-glutamic acid) (PDEA) and poly(D-aspartic acid) (PDDA); and comprises a second anionic polymer selected from poly(L-glutamic acid) (PLEA) and poly(L-aspartic acid) (PLDA). In some cases, the cationic polymer composition comprises a first cationic polymer selected from poly(D-arginine), poly(D-lysine), poly(D-histidine), poly(D-ornithine), and poly(D-citrulline); and comprises a second cationic polymer selected from poly(L-arginine), poly(L-lysine), poly(L-histidine), poly(L-ornithine), and poly(L-citrulline). In some cases, the polymers of D-isomers of an anionic amino acid are present at a ratio, relative to said polymers of L-isomers of an anionic amino acid, in a range of from 10:1 to 1:10. In some cases, the polymers of D-isomers of a cationic amino acid are present at a ratio, relative to said polymers of L-isomers of a cationic amino acid, in a range of from 10:1 to 1:10.

Nanoparticle Components (Delayed and/or Extended Payload Release)

In some embodiments, timing of payload release can be controlled by selecting particular types of proteins, e.g., as part of the core (e.g., part of a cationic polypeptide composition, part of a cationic polymer composition, and/or part of an anionic polymer composition). For example, it may be desirable to delay payload release for a particular range of time, or until the payload is present at a particular cellular location (e.g., cytosol, nucleus, lysosome, endosome) or under a particular condition (e.g., low pH, high pH, etc.). As such, in some cases a protein is used (e.g., as part of the core) that is susceptible to a specific protein activity (e.g., enzymatic activity), e.g., is a substrate for a specific protein activity (e.g., enzymatic activity), and this is in contrast to being susceptible to general ubiquitous cellular machinery, e.g., general degradation machinery. A protein that is susceptible to a specific protein activity is referred to herein as an ‘enzymatically susceptible protein’ (ESP). Illustrative examples of ESPs include but are not limited to: (i) proteins that are substrates for matrix metalloproteinase (MMP) activity (an example of an extracellular activity), e.g., a protein that includes a motif recognized by an MMP; (ii) proteins that are substrates for cathepsin activity (an example of an intracellular endosomal activity), e.g., a protein that includes a motif recognized by a cathepsin; and (iii) proteins such as histone tails peptides (HTPs) that are substrates for methyltransferase and/or acetyltransferase activity (an example of an intracellular nuclear activity), e.g., a protein that includes a motif that can be enzymatically methylated/de-methylated and/or a motif that can be enzymatically acetylated/de-acetylated. For example, in some cases a nucleic acid payload is condensed with a protein (such as a histone tails peptide) that is a substrate for acetyltransferase activity, and acetylation of the protein causes the protein to release the payload—as such, one can exercise control over payload release by choosing to use a protein that is more or less susceptible to acetylation.

In some cases, a core of a subject nanoparticle includes an enzymatically neutral polypeptide (ENP), which is a polypeptide homopolymer (i.e., a protein having a repeat sequence) where the polypeptide does not have a particular activity and is neutral. For example, unlike NLS sequences and HTPs, both of which have a particular activity, ENPs do not.

In some cases, a core of a subject nanoparticle includes an enzymatically protected polypeptide (EPP), which is a protein that is resistant to enzymatic activity. Examples of PPs include but are not limited to: (i) polypeptides that include D-isomer amino acids (e.g., D-isomer polymers), which can resist proteolytic degradation; and (ii) self-sheltering domains such as a polyglutamine repeat domains (e.g., QQQQQQQQQQ) (SEQ ID NO: 170).

By controlling the relative amounts of susceptible proteins (ESPs), neutral proteins (ENPs), and protected proteins (EPPs), that are part of a subject nanoparticle (e.g., part of the nanoparticle core), one can control the release of payload. For example, use of more ESPs can in general lead to quicker release of payload than use of more EPPs. In addition, use of more ESPs can in general lead to release of payload that depends upon a particular set of conditions/circumstances, e.g., conditions/circumstances that lead to activity of proteins (e.g., enzymes) to which the ESP is susceptible.

In some cases, ratios of carrier molecules relative to one another are modulating while designing delivery vehicle (e.g., nanoparticle) formulations. Term “carrier molecules” refers to components of the delivery vehicle that are not the payload or targeting ligand—for example: anionic polymer, cationic polymer, cationic polypeptide (e.g., HTP), a lipid, and the like.

Anionic Polymer Composition (e.g., of a Nanoparticle)

An anionic polymer composition can include one or more anionic amino acid polymers. For example, in some cases a subject anionic polymer composition includes a polymer selected from: poly(glutamic acid)(PEA), poly(aspartic acid)(PDA), and a combination thereof. In some cases a given anionic amino acid polymer can include a mix of aspartic and glutamic acid residues. Each polymer can be present in the composition as a polymer of L-isomers or D-isomers, where D-isomers are more stable in a target cell because they take longer to degrade. Thus, inclusion of D-isomer poly(amino acids) in the nanoparticle core delays degradation of the core and subsequent payload release. A suitable ratio of D to L isomer polypeptides can be determined by performing a robotic screen utilizing a formulator app, such as shown in FIG. 19B. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases an anionic polymer composition of a subject nanoparticle includes polymers of D-isomers and polymers of L-isomers of an anionic amino acid polymer (e.g., poly(glutamic acid)(PEA) and poly(aspartic acid)(PDA)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases an anionic polymer composition includes a first anionic polymer (e.g., amino acid polymer) that is a polymer of D-isomers (e.g., selected from poly(D-glutamic acid) (PDEA) and poly(D-aspartic acid) (PDDA)); and includes a second anionic polymer (e.g., amino acid polymer) that is a polymer of L-isomers (e.g., selected from poly(L-glutamic acid) (PLEA) and poly(L-aspartic acid) (PLDA)). In some cases the ratio of the first anionic polymer (D-isomers) to the second anionic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

In some embodiments, an anionic polymer composition of a core of a subject nanoparticle includes (e.g., in addition to or in place of any of the foregoing examples of anionic polymers) a glycosaminoglycan, a glycoprotein, a polysaccharide, poly(mannuronic acid), poly(guluronic acid), heparin, heparin sulfate, chondroitin, chondroitin sulfate, keratan, keratan sulfate, aggrecan, poly(glucosamine), or an anionic polymer that comprises any combination thereof.

In some embodiments, an anionic polymer within the core can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases an anionic polymer includes poly(glutamic acid) with a molecular weight of approximately 15 kDa.

In some cases, an anionic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes a cysteine residue. In some cases the anionic amino acid polymer includes cysteine residue on the N- and/or C-terminus. In some cases the anionic amino acid polymer includes an internal cysteine residue.

In some cases, an anionic amino acid polymer includes (and/or is conjugated to) a nuclear localization signal (NLS) (described in more detail below). Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

In some cases, an anionic polymer is added prior to a cationic polymer when generating a subject nanoparticle core. In some cases, the matrix output of a robotic synthesis of various D:L isomer ratios of constituent polypeptides in a given nanoparticle screen can be used as an input variable for subsequent machine learning and recursive optimization approaches of additional degrees of freedom of the nanoparticle platform as shown in FIGS. 13C-13H, with finite biological and physicochemical data outputs.

Cationic Polymer Composition (e.g., of a Nanoparticle)

A cationic polymer composition can include one or more cationic amino acid polymers. For example, in some cases a subject cationic polymer composition includes a polymer selected from: poly(arginine)(PR), poly(lysine)(PK), poly(histidine)(PH), poly(ornithine), poly(citrulline), and a combination thereof. In some cases a given cationic amino acid polymer can include a mix of arginine, lysine, histidine, ornithine, and citrulline residues (in any convenient combination). Each polymer can be present in the composition as a polymer of L-isomers or D-isomers, where D-isomers are more stable in a target cell because they take longer to degrade. Thus, inclusion of D-isomer poly(amino acids) in the nanoparticle core delays degradation of the core and subsequent payload release. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases a cationic polymer composition of a subject nanoparticle includes polymers of D-isomers and polymers of L-isomers of an cationic amino acid polymer (e.g., poly(arginine)(PR), poly(lysine)(PK), poly(histidine)(PH), poly(ornithine), poly(citrulline)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases a cationic polymer composition includes a first cationic polymer (e.g., amino acid polymer) that is a polymer of D-isomers (e.g., selected from poly(D-arginine), poly(D-lysine), poly(D-histidine), poly(D-ornithine), and poly(D-citrulline)); and includes a second cationic polymer (e.g., amino acid polymer) that is a polymer of L-isomers (e.g., selected from poly(L-arginine), poly(L-lysine), poly(L-histidine), poly(L-ornithine), and poly(L-citrulline)). In some cases the ratio of the first cationic polymer (D-isomers) to the second cationic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, a cationic polymer composition of a core of a subject nanoparticle includes (e.g., in addition to or in place of any of the foregoing examples of cationic polymers) poly(ethylenimine), poly(amidoamine) (PAMAM), poly(aspartamide), polypeptoids (e.g., for forming “spiderweb”-like branches for core condensation), a charge-functionalized polyester, a cationic polysaccharide, an acetylated amino sugar, chitosan, or a cationic polymer that comprises any combination thereof (e.g., in linear or branched forms).

In some embodiments, a cationic polymer within the core can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases a cationic polymer includes poly(L-arginine), e.g., with a molecular weight of approximately 29 kDa. As another example, in some cases a cationic polymer includes linear poly(ethylenimine) with a molecular weight of approximately 25 kDa (PEI). As another example, in some cases a cationic polymer includes branched poly(ethylenimine) with a molecular weight of approximately 10 kDa. As another example, in some cases a cationic polymer includes branched poly(ethylenimine) with a molecular weight of approximately 70 kDa. In some cases a cationic polymer includes PAMAM.

In some cases, a cationic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes a cysteine residue. In some cases the cationic amino acid polymer includes cysteine residue on the N- and/or C-terminus. In some cases the cationic amino acid polymer includes an internal cysteine residue.

In some cases, a cationic amino acid polymer includes (and/or is conjugated to) a nuclear localization signal (NLS) (described in more detail below). Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes (and/or is conjugated to) one or more (e.g., two or more, three or more, or four or more) NLSs. In some cases the cationic amino acid polymer includes an NLS on the N- and/or C-terminus. In some cases the cationic amino acid polymer includes an internal NLS.

Cationic Polypeptide Composition (e.g., of a Nanoparticle)

In some embodiments the cationic polypeptide composition of a nanoparticle can mediate stability, subcellular compartmentalization, and/or payload release. As one example, fragments of the N-terminus of histone proteins, referred to generally as histone tail peptides, within a subject nanoparticle core are in some case not only capable of being deprotonated by various histone modifications, such as in the case of histone acetyltransferase-mediated acetylation, but may also mediate effective nuclear-specific unpackaging of components (e.g., a payload) of a nanoparticle core. In some cases a cationic polypeptide composition includes a histone and/or histone tail peptide (e.g., a cationic polypeptide can be a histone and/or histone tail peptide). In some cases a cationic polypeptide composition includes an NLS-containing peptide (e.g., a cationic polypeptide can be an NLS-containing peptide). In some cases, a cationic polypeptide composition includes one or more NLS-containing peptides separated by cysteine residues to facilitate crosslinking. In some cases a cationic polypeptide composition includes a peptide that includes a mitochondrial localization signal (e.g., a cationic polypeptide can be a peptide that includes a mitochondrial localization signal).

Histone Tail Peptide (HTPs)

In some embodiments a cationic polypeptide composition (e.g., of a subject nanoparticle) includes a histone peptide or a fragment of a histone peptide, such as an N-terminal histone tail (e.g., a histone tail of an H1, H2 (e.g., H2A, H2AX, H2B), H3, or H4 histone protein). A tail fragment of a histone protein is referred to herein as a histone tail peptide (HTP). Because such a protein (a histone and/or HTP) can condense with a nucleic acid payload as part of the core of a subject nanoparticle, a core that includes one or more histones or HTPs (e.g., as part of the cationic polypeptide composition) is sometimes referred to herein as a nucleosome-mimetic core. Histones and/or HTPs can be included as monomers, and in some cases form dimers, trimers, tetramers and/or octamers when condensing a nucleic acid payload into a nanoparticle core. In some cases HTPs are not only capable of being deprotonated by various histone modifications, such as in the case of histone acetyltransferase-mediated acetylation, but may also mediate effective nuclear-specific unpackaging of components of the core (e.g., release of a payload). Trafficking of a core that includes a histone and/or HTP may be reliant on alternative endocytotic pathways utilizing retrograde transport through the Golgi and endoplasmic reticulum. Furthermore, some histones include an innate nuclear localization sequence and inclusion of an NLS in the core can direct the core (including the payload) to the nucleus of a target cell.

In some embodiments a subject cationic polypeptide composition includes a protein having an amino acid sequence of an H2A, H2AX, H2B, H3, or H4 protein. In some cases a subject cationic polypeptide composition includes a protein having an amino acid sequence that corresponds to the N-terminal region of a histone protein. For example, the fragment (an HTP) can include the first 5, 10, 15, 20, 25, 30, 35, 40, 45, or 50 N-terminal amino acids of a histone protein. In some cases, a subject HTP includes from 5-50 amino acids (e.g., from 5-45, 5-40, 5-35, 5-30, 5-25, 5-20, 8-50, 8-45, 8-40, 8-35, 8-30, 10-50, 10-45, 10-40, 10-35, or 10-30 amino acids) from the N-terminal region of a histone protein. In some cases a subject a cationic polypeptide includes from 5-150 amino acids (e.g., from 5-100, 5-50, 5-35, 5-30, 5-25, 5-20, 8-150, 8-100, 8-50, 8-40, 8-35, 8-30, 10-150, 10-100, 10-50, 10-40, 10-35, or 10-30 amino acids).

In some cases a cationic polypeptide (e.g., a histone or HTP, e.g., H1, H2, H2A, H2AX, H2B, H3, or H4) of a cationic polypeptide composition includes a post-translational modification (e.g., in some cases on one or more histidine, lysine, arginine, or other complementary residues). For example, in some cases the cationic polypeptide is methylated (and/or susceptible to methylation/demethylation), acetylated (and/or susceptible to acetylation/deacetylation), crotonylated (and/or susceptible to crotonylation/decrotonylation), ubiquitinylated (and/or susceptible to ubiquitinylation/deubiquitinylation), phosphorylated (and/or susceptible to phosphorylation/dephosphorylation), SUMOylated (and/or susceptible to SUMOylation/deSUMOylation), farnesylated (and/or susceptible to farnesylation/defarnesylation), sulfated (and/or susceptible to sulfation/desulfation) or otherwise post-translationally modified. In some cases a cationic polypeptide (e.g., a histone or HTP, e.g., H1, H2, H2A, H2AX, H2B, H3, or H4) of a cationic polypeptide composition is p300/CBP substrate (e.g., see example HTPs below, e.g., SEQ ID NOs: 129-130). In some cases a cationic polypeptide (e.g., a histone or HTP, e.g., H1, H2, H2A, H2AX, H2B, H3, or H4) of a cationic polypeptide composition includes one or more thiol residues (e.g., can include a cysteine and/or methionine residue) that is sulfated or susceptible to sulfation (e.g., as a thiosulfate sulfurtransferase substrate). In some cases a cationic polypeptide (e.g., a histone or HTP, e.g., H1, H2, H2A, H2AX, H2B, H3, or H4) of a cationic polypeptide is amidated on the C-terminus. Histones H2A, H2B, H3, and H4 (and/or HTPs) may be monomethylated, dimethylated, or trimethylated at any of their lysines to promote or suppress transcriptional activity and alter nuclear-specific release kinetics.

A cationic polypeptide can be synthesized with a desired modification or can be modified in an in vitro reaction. Alternatively, a cationic polypeptide (e.g., a histone or HTP) can be expressed in a cell population and the desired modified protein can be isolated/purified. In some cases the cationic polypeptide composition of a subject nanoparticle includes a methylated HTP, e.g., includes the HTP sequence of H3K4(Me3)—includes the amino acid sequence set forth as SEQ ID NO: 75 or 88). In some cases a cationic polypeptide (e.g., a histone or HTP, e.g., H1, H2, H2A, H2AX, H2B, H3, or H4) of a cationic polypeptide composition includes a C-terminal amide.

Examples of Histones and HTPs

Examples include but are not limited to the following sequences:

H2A
(SEQ ID NO: 62)
SGRGKQGGKARAKAKTRSSR [1-20]
(SEQ ID NO: 63)
SGRGKQGGKARAKAKTRSSRAGLQFPVGRVHRLLRKGGG [1-39]
(SEQ ID NO: 64)
MSGRGKQGGKARAKAKTRSSRAGLQFPVGRVHRLLRKGNYAERVGAGAPVYLAAVLEYL
TAEILELAGNAARDNKKTRIIPRHLQLAIRNDEELNKLLGKVTIAQGGVLPNIQAVLLPKKTE
SHHKAKGK [1-130]
H2AX
(SEQ ID NO: 65)
CKATQASQEY [134-143]
(SEQ ID NO: 66)
KKTSATVGPKAPSGGKKATQASQEY [KK 120-129]
(SEQ ID NO: 67)
MSGRGKTGGKARAKAKSRSSRAGLQFPVGRVHRLLRKGHYAERVGAGAPVYLAAVLEYL
TAEILELAGNAARDNKKTRIIPRHLQLAIRNDEELNKLLGGVTIAQGGVLPNIQAVLLPKKTS
ATVGPKAPSGGKKATQASQEY [1-143]
H2B
(SEQ ID NO: 68)
PEPA - K(cr) - SAPAPK [1-11 H2BK5(cr)]
[cr: crotonylated (crotonylation)]
(SEQ ID NO: 69)
PEPAKSAPAPK [1-11]
(SEQ ID NO: 70)
AQKKDGKKRKRSRKE [21-35]
(SEQ ID NO: 71)
MPEPAKSAPAPKKGSKKAVTKAQKKDGKKRKRSRKESYSIYVYKVLKQVHPDTGISSKAM
GIMNSFVNDIFERIAGEASRLAHYNKRSTITSREIQTAVRLLLPGELAKHAVSEGTKAVTKYT
SSK [1-126]
H3
(SEQ ID NO: 72)
ARTKQTAR [1-8]
(SEQ ID NO: 73)
ART - K(Me1) - QTARKS [1-8 H3K4(Me1)]
(SEQ ID NO: 74)
ART - K(Me2) - QTARKS [1-8 H3K4(Me2)]
(SEQ ID NO: 75)
ART - K(Me3) - QTARKS [1-8 H3K4(Me3)]
(SEQ ID NO: 76)
ARTKQTARK - pS - TGGKA [1-15 H3pS10]
(SEQ ID NO: 77)
ARTKQTARKSTGGKAPRKWC - NH2 [1-18 WC, amide]
(SEQ ID NO: 78)
ARTKQTARKSTGG - K(Ac) - APRKQ [1-19 H3K14(Ac)]
(SEQ ID NO: 79)
ARTKQTARKSTGGKAPRKQL [1-20]
(SEQ ID NO: 80)
ARTKQTAR - K(Ac) - STGGKAPRKQL [1-20 H3K9(Ac)]
(SEQ ID NO: 81)
ARTKQTARKSTGGKAPRKQLA [1-21]
(SEQ ID NO: 82)
ARTKQTAR - K(Ac) - STGGKAPRKQLA [1-21 H3K9(Ac)]
(SEQ ID NO: 83)
ARTKQTAR - K(Me2) - STGGKAPRKQLA [1-21 H3K9(Me1)]
(SEQ ID NO: 84)
ARTKQTAR - K(Me2) - STGGKAPRKQLA [1-21 H3K9(Me2)]
(SEQ ID NO: 85)
ARTKQTAR - K(Me2) - STGGKAPRKQLA [1-21 H3K9(Me3)]
(SEQ ID NO: 86)
ART - K(Me1) - QTARKSTGGKAPRKQLA [1-21 H3K4(Me1)]
(SEQ ID NO: 87)
ART - K(Me2) - QTARKSTGGKAPRKQLA [1-21 H3K4(Me2)]
(SEQ ID NO: 88)
ART - K(Me3) - QTARKSTGGKAPRKQLA [1-21 H3K4(Me3)]
(SEQ ID NO: 89)
ARTKQTAR - K(Ac) - ps - TGGKAPRKQLA [1-21 H3K9(Ac), pS10]
(SEQ ID NO: 90)
ART - K(Me3) - QTAR - K(Ac) - pS - TGGKAPRKQLA
[1-21 H3K4(Me3), K9(Ac), pS10]
(SEQ ID NO: 91)
ARTKQTARKSTGGKAPRKQLAC [1-21 Cys]
(SEQ ID NO: 92)
ARTKQTAR - K(Ac) - STGGKAPRKQLATKA [1-24 H3K9(Ac)]
(SEQ ID NO: 93)
ARTKQTAR - K(Me3) - STGGKAPRKQLATKA [1-24 H3K9(Me3)]
(SEQ ID NO: 94)
ARTKQTARKSTGGKAPRKQLATKAA [1-25]
(SEQ ID NO: 95)
ART - K(Me3) - QTARKSTGGKAPRKQLATKAA [1-25 H3K4(Me3)]
(SEQ ID NO: 96)
TKQTAR - K(Me1) - STGGKAPR [3-17 H3K9(Me1)]
(SEQ ID NO: 97)
TKQTAR - K(Me2) - STGGKAPR [3-17 H3K9(Me2)]
(SEQ ID NO: 98)
TKQTAR - K(Me3) - STGGKAPR [3-17 H3K9(Me3)]
(SEQ ID NO: 99)
KSTGG - K(Ac) - APRKQ [9-19 H3K14(Ac)]
(SEQ ID NO: 100)
QTARKSTGGKAPRKQLASK [5-23]
(SEQ ID NO: 101)
APRKQLATKAARKSAPATGGVKKPH [15-39]
(SEQ ID NO: 102)
ATKAARKSAPATGGVKKPHRYRPG [21-44]
(SEQ ID NO: 103)
KAARKSAPA [23-31]
(SEQ ID NO: 104)
KAARKSAPATGG [23-34]
(SEQ ID NO: 105)
KAARKSAPATGGC [23-34 Cys]
(SEQ ID NO: 106)
KAAR - K(Ac) - SAPATGG [H3K27(Ac)]
(SEQ ID NO: 107)
KAAR - K(Me1) - SAPATGG [H3K37(Me1)]
(SEQ ID NO: 108)
KAAR - K(Me2) - SAPATGG [H3K37(Me2)]
(SEQ ID NO: 109)
KAAR - K(Me3) - SAPATGG [H3K37(Me3)]
(SEQ ID NO: 110)
AT - K(Ac) - AARKSAPSTGGVKKPHRYRPG [21-44 H3K23(Ac)]
(SEQ ID NO: 111)
ATKAARK - pS - APATGGVKKPHRYRPG [21-44 pS28]
(SEQ ID NO: 112)
ARTKQTARKSTGGKAPRKQLATKAARKSAPATGGV [1-35]
(SEQ ID NO: 113)
STGGV - K(Me1) - KPHRY [31-41 H3K36(Me1)]
(SEQ ID NO: 114)
STGGV - K(Me2) - KPHRY [31-41 H3K36(Me2)]
(SEQ ID NO: 115)
STGGV - K(Me3) - KPHRY [31-41 H3K36(Me3)]
(SEQ ID NO: 116)
GTVALREIRRYQ - K(Ac) - STELLIR [44-63 H3K56(Ac)]
(SEQ ID NO: 117)
ARTKQTARKSTGGKAPRKQLATKAARKSAPATGGVKKPHRYRPGTVALRE [1-50]
(SEQ ID NO: 118)
TELLIRKLPFQRLVREIAQDF - K(Me1) - TDLRFQSAAI [H3K79(Me1)]
(SEQ ID NO: 119)
EIAQDFKTDLR [73-83]
(SEQ ID NO: 120)
EIAQDF - K(Ac) - TDLR [73-83 H3K79(Ac)]
(SEQ ID NO: 121)
EIAQDF - K(Me3) - TDLR [73-83 H3K79(Me3)]
(SEQ ID NO: 122)
RLVREIAQDFKTDLRFQSSAV [69-89]
(SEQ ID NO: 123)
RLVREIAQDFK - (Me1) - TDLRFQSSAV [69-89 H3K79 (Me1), amide]
(SEQ ID NO: 124)
RLVREIAQDFK - (Me2) - TDLRFQSSAV [69-89 H3K79 (Me2), amide]
(SEQ ID NO: 125)
RLVREIAQDFK - (Me3) - TDLRFQSSAV [69-89 H3K79 (Me3), amide]
(SEQ ID NO: 126)
KRVTIMPKDIQLARRIRGERA [116-136]
(SEQ ID NO: 127)
MARTKQTARKSTGGKAPRKQLATKVARKSAPATGGVKKPHRYRPGTVALREIRRYQKSTE
LLIRKLPFQRLMREIAQDFKTDLRFQSSAVMALQEACESYLVGLFEDTNLCVIHAKRVTIMP
KDIQLARRIRGERA [1-136]
H4
(SEQ ID NO: 128)
SGRGKGG [1-7]
(SEQ ID NO: 129)
RGKGGKGLGKGA [4-12]
(SEQ ID NO: 130)
SGRGKGGKGLGKGGAKRHRKV [1-21]
(SEQ ID NO: 131)
KGLGKGGAKRHRKVLRDNWC - NH2 [8-25 WC, amide]
(SEQ ID NO: 132)
SGRG - K(Ac) - GG - K(Ac) - GLG - K(Ac) - GGA - K(Ac) -
RHRKVLRDNGSGSK [1-25 H4K5, 8, 12, 16(Ac)]
(SEQ ID NO: 133)
SGRGKGGKGLGKGGAKRHRK - NH2 [1-20 H4 PRMT7 (protein arginine
methyltransferase 7) Substrate, M1]
(SEQ ID NO: 134)
SGRG - K(Ac) - GGKGLGKGGAKRHRK [1-20 H4K5 (Ac)]
(SEQ ID NO: 135)
SGRGKGG - K(Ac) - GLGKGGAKRHRK [1-20 H4K8 (Ac)]
(SEQ ID NO: 136)
SGRGKGGKGLG - K(Ac) - GGAKRHRK [1-20 H4K12 (Ac)]
(SEQ ID NO: 137)
SGRGKGGKGLGKGGA - K(Ac) - RHRK [1-20 H4K16 (Ac)]
(SEQ ID NO: 138)
KGLGKGGAKRHRKVLRDNWC - NH2 [1-25 WC, amide]
(SEQ ID NO: 139)
MSGRGKGGKGLGKGGAKRHRKVLRDNIQGITKPAIRRLARRGGVKRISGLIYEETRGVLKV
FLENVIRDAVTYTEHAKRKTVTAMDVVYALKRQGRTLYGFGG [1-103]

As such, a cationic polypeptide of a subject cationic polypeptide composition can include an amino acid sequence having the amino acid sequence set forth in any of SEQ ID NOs: 62-139. In some cases a cationic polypeptide of subject a cationic polypeptide composition includes an amino acid sequence having 80% or more sequence identity (e.g., 85% or more, 90% or more, 95% or more, 98% or more, 99% or more, or 100% sequence identity) with the amino acid sequence set forth in any of SEQ ID NOs: 62-139. In some cases a cationic polypeptide of subject a cationic polypeptide composition includes an amino acid sequence having 90% or more sequence identity (e.g., 95% or more, 98% or more, 99% or more, or 100% sequence identity) with the amino acid sequence set forth in any of SEQ ID NOs: 62-139. The cationic polypeptide can include any convenient modification, and a number of such contemplated modifications are discussed above, e.g., methylated, acetylated, crotonylated, ubiquitinylated, phosphorylated, SUMOylated, farnesylated, sulfated, and the like.

In some cases a cationic polypeptide of a cationic polypeptide composition includes an amino acid sequence having 80% or more sequence identity (e.g., 85% or more, 90% or more, 95% or more, 98% or more, 99% or more, or 100% sequence identity) with the amino acid sequence set forth in SEQ ID NO: 94. In some cases a cationic polypeptide of a cationic polypeptide composition includes an amino acid sequence having 95% or more sequence identity (e.g., 98% or more, 99% or more, or 100% sequence identity) with the amino acid sequence set forth in SEQ ID NO: 94. In some cases a cationic polypeptide of a cationic polypeptide composition includes the amino acid sequence set forth in SEQ ID NO: 94. In some cases a cationic polypeptide of a cationic polypeptide composition includes the sequence represented by H3K4(Me3) (SEQ ID NO: 95), which comprises the first 25 amino acids of the human histone 3 protein, and tri-methylated on the lysine 4 (e.g., in some cases amidated on the C-terminus).

In some embodiments a cationic polypeptide (e.g., a histone or HTP, e.g., H1, H2, H2A, H2AX, H2B, H3, or H4) of a cationic polypeptide composition includes a cysteine residue, which can facilitate conjugation to: a cationic (or in some cases anionic) amino acid polymer, a linker, an NLS, and/or other cationic polypeptides (e.g., in some cases to form a branched histone structure). For example, a cysteine residue can be used for crosslinking (conjugation) via sulfhydryl chemistry (e.g., a disulfide bond) and/or amine-reactive chemistry. In some cases the cysteine residue is internal. In some cases the cysteine residue is positioned at the N-terminus and/or C-terminus. In some cases, a cationic polypeptide (e.g., a histone or HTP, e.g., H1, H2, H2A, H2AX, H2B, H3, or H4) of a cationic polypeptide composition includes a mutation (e.g., insertion or substitution) that adds a cysteine residue. Examples of HTPs that include a cysteine include but are not limited to:

(SEQ ID NO: 140)
CKATQASQEY - from H2AX
(SEQ ID NO: 141)
ARTKQTARKSTGGKAPRKQLAC - from H3
(SEQ ID NO: 142)
ARTKQTARKSTGGKAPRKWC
(SEQ ID NO: 143)
KAARKSAPATGGC - from H3
(SEQ ID NO: 144)
KGLGKGGAKRHRKVLRDNWC - from H4
(SEQ ID NO: 145)
MARTKQTARKSTGGKAPRKQLATKVARKSAPATGGVKKPHRYRPGTVALR
EIRRYQKSTELLIRKLPFQRLMREIAQDFKTDLRFQSSAVMALQEACESY
LVGLFEDTNLCVIHAKRVTIMPKDIQLARRIRGERA - from H3

In some embodiments a cationic polypeptide (e.g., a histone or HTP, e.g., H1, H2, H2A, H2AX, H2B, H3, or H4) of a cationic polypeptide composition is conjugated to a cationic (and/or anionic) amino acid polymer of the core of a subject nanoparticle. As an example, a histone or HTP can be conjugated to a cationic amino acid polymer (e.g., one that includes poly(lysine)), via a cysteine residue, e.g., where the pyridyl disulfide group(s) of lysine(s) of the polymer are substituted with a disulfide bond to the cysteine of a histone or HTP.

Modified/Branching Structure

In some embodiments a cationic polypeptide of a subject a cationic polypeptide composition has a linear structure. In some embodiments a cationic polypeptide of a subject a cationic polypeptide composition has a branched structure.

For example, in some cases, a cationic polypeptide (e.g., HTPs, e.g., HTPs with a cysteine residue) is conjugated (e.g., at its C-terminus) to the end of a cationic polymer (e.g., poly(L-arginine), poly(D-lysine), poly(L-lysine), poly(D-lysine)), thus forming an extended linear polypeptide. In some cases, one or more (two or more, three or more, etc.) cationic polypeptides (e.g., HTPs, e.g., HTPs with a cysteine residue) are conjugated (e.g., at their C-termini) to the end(s) of a cationic polymer (e.g., poly(L-arginine), poly(D-lysine), poly(L-lysine), poly(D-lysine)), thus forming an extended linear polypeptide. In some cases the cationic polymer has a molecular weight in a range of from 4,500-150,000 Da).

As another example, in some cases, one or more (two or more, three or more, etc.) cationic polypeptides (e.g., HTPs, e.g., HTPs with a cysteine residue) are conjugated (e.g., at their C-termini) to the side-chains of a cationic polymer (e.g., poly(L-arginine), poly(D-lysine), poly(L-lysine), poly(D-lysine)), thus forming a branched structure (branched polypeptide).

Formation of a branched structure by components of the nanoparticle core (e.g., components of a subject cationic polypeptide composition) can in some cases increase the amount of core condensation (e.g., of a nucleic acid payload) that can be achieved. Thus, in some cases it is desirable to used components that form a branched structure. Various types of branches structures are of interest, and examples of branches structures that can be generated (e.g., using subject cationic polypeptides such as HTPs, e.g., HTPs with a cysteine residue; peptoids, polyamides, and the like) include but are not limited to: brush polymers, webs (e.g., spider webs), graft polymers, star-shaped polymers, comb polymers, polymer networks, dendrimers, and the like.

In some cases, a branched structure includes from 2-30 cationic polypeptides (e.g., HTPs) (e.g., from 2-25, 2-20, 2-15, 2-10, 2-5, 4-30, 4-25, 4-20, 4-15, or 4-10 cationic polypeptides), where each can be the same or different than the other cationic polypeptides of the branched structure. In some cases the cationic polymer has a molecular weight in a range of from 4,500-150,000 Da). In some cases, 5% or more (e.g., 10% or more, 20% or more, 25% or more, 30% or more, 40% or more, or 50% or more) of the side-chains of a cationic polymer (e.g., poly(L-arginine), poly(D-lysine), poly(L-lysine), poly(D-lysine)) are conjugated to a subject cationic polypeptide (e.g., HTP, e.g., HTP with a cysteine residue). In some cases, up to 50% (e.g., up to 40%, up to 30%, up to 25%, up to 20%, up to 15%, up to 10%, or up to 5%) of the side-chains of a cationic polymer (e.g., poly(L-arginine), poly(D-lysine), poly(L-lysine), poly(D-lysine)) are conjugated to a subject cationic polypeptide (e.g., HTP, e.g., HTP with a cysteine residue). Thus, an HTP can be branched off of the backbone of a polymer such as a cationic amino acid polymer.

In some cases formation of branched structures can be facilitated using components such as peptoids (polypeptoids), polyamides, dendrimers, and the like. For example, in some cases peptoids (e.g., polypeptoids) are used as a component of a nanoparticle core, e.g., in order to generate a web (e.g., spider web) structure, which can in some cases facilitate condensation of the nanoparticle core.

One or more of the natural or modified polypeptide sequences herein may be modified with terminal or intermittent arginine, lysine, or histidine sequences. In one embodiment, each polypeptide is included in equal amine molarities within a nanoparticle core. In this embodiment, each polypeptide's C-terminus can be modified with 5R (5 arginines). In some embodiments, each polypeptide's C-terminus can be modified with 9R (9 arginines). In some embodiments, each polypeptide's N-terminus can be modified with 5R (5 arginines). In some embodiments, each polypeptide's N-terminus can be modified with 9R (9 arginines). In some cases, an H2A, H2B, H3 and/or H4 histone fragment (e.g., HTP) are each bridged in series with a FKFL Cathepsin B proteolytic cleavage domain or RGFFP Cathepsin D proteolytic cleavage domain. In some cases, an H2A, H2B, H3 and/or H4 histone fragment (e.g., HTP) can be bridged in series by a 5R (5 arginines), 9R (9 arginines), 5K (5 lysines), 9K (9 lysines), 5H (5 histidines), or 9H (9 histidines) cationic spacer domain. In some cases, one or more H2A, H2B, H3 and/or H4 histone fragments (e.g., HTPs) are disulfide-bonded at their N-terminus to protamine.

To illustrate how to generate a branched histone structure, example methods of preparation are provided. One example of such a method includes the following: covalent modification of equimolar ratios of Histone H2AX [134-143], Histone H3 [1-21 Cys], Histone H3 [23-34 Cys], Histone H4 [8-25 WC] and SV40 T-Ag-derived NLS can be performed in a reaction with 10% pyridyl disulfide modified poly(L-Lysine) [MW=5400, 18000, or 45000 Da; n=30, 100, or 250]. In a typical reaction, a 29 μL aqueous solution of 700 μM Cys-modified histone/NLS (20 nmol) can be added to 57 μL of 0.2 M phosphate buffer (pH 8.0). Second, 14 μL of 100 μM pyridyl disulfide protected poly(lysine) solution can then be added to the histone solution bringing the final volume to 100 μL with a 1:2 ratio of pyridyl disulfide groups to Cysteine residues. This reaction can be carried out at room temperature for 3 h. The reaction can be repeated four times and degree of conjugation can be determined via absorbance of pyridine-2-thione at 343 nm.

As another example, covalent modification of a 0:1, 1:4, 1:3, 1:2, 1:1, 1:2, 1:3, 1:4, or 1:0 molar ratio of Histone H3 [1-21 Cys] peptide and Histone H3 [23-34 Cys] peptide can be performed in a reaction with 10% pyridyl disulfide modified poly(L-Lysine) or poly(L-Arginine) [MW=5400, 18000, or 45000 Da; n=30, 100, or 250]. In a typical reaction, a 29 μL aqueous solution of 700 μM Cys-modified histone (20 nmol) can be added to 57 μL of 0.2 M phosphate buffer (pH 8.0). Second, 14 μL of 100 μM pyridyl disulfide protected poly(lysine) solution can then be added to the histone solution bringing the final volume to 100 with a 1:2 ratio of pyridyl disulfide groups to Cysteine residues. This reaction can be carried out at room temperature for 3 h. The reaction can be repeated four times and degree of conjugation can be determined via absorbance of pyridine-2-thione at 343 nm.

In some cases, an anionic polymer is conjugated to a targeting ligand.

Nuclear Localization Sequence (NLS)

In some embodiments a cationic polypeptide (e.g., a histone or HTP, e.g., H1, H2, H2A, H2AX, H2B, H3, or H4) of a cationic polypeptide composition includes (and/or is conjugated to) one or more (e.g., two or more, three or more, or four or more) nuclear localization sequences (NLSs). Thus in some cases the cationic polypeptide composition of a subject nanoparticle includes a peptide that includes an NLS. In some cases a histone protein (or an HTP) of a subject nanoparticle includes one or more (e.g., two or more, three or more) natural nuclear localization signals (NLSs). In some cases a histone protein (or an HTP) of a subject nanoparticle includes one or more (e.g., two or more, three or more) NLSs that are heterologous to the histone protein (i.e., NLSs that do not naturally occur as part of the histone/HTP, e.g., an NLS can be added by humans). In some cases the HTP includes an NLS on the N- and/or C-terminus.

In some embodiments a cationic amino acid polymer (e.g., poly(arginine)(PR), poly(lysine)(PK), poly(histidine)(PH), poly(ornithine), poly(citrulline), poly(D-arginine)(PDR), poly(D-lysine)(PDK), poly(D-histidine)(PDH), poly(D-ornithine), poly(D-citrulline), poly(L-arginine)(PLR), poly(L-lysine)(PLK), poly(L-histidine)(PLH), poly(L-ornithine), or poly(L-citrulline)) of a cationic polymer composition includes (and/or is conjugated to) one or more (e.g., two or more, three or more, or four or more) NLSs. In some cases the cationic amino acid polymer includes an NLS on the N- and/or C-terminus. In some cases the cationic amino acid polymer includes an internal NLS.

In some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), or poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

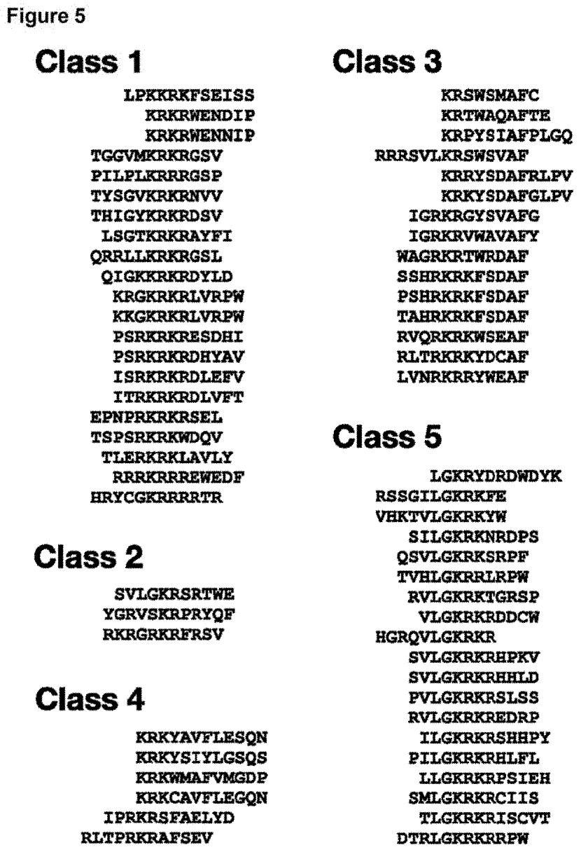

Any convenient NLS can be used (e.g., conjugated to a histone, an HTP, a cationic amino acid polymer, an anionic amino acid polymer, and the like). Examples include, but are not limited to Class 1 and Class 2 ‘monopartite NLSs’, as well as NLSs of Classes 3-5 (see, e.g., FIG. 5, which is adapted from Kosugi et al., J Biol Chem. 2009 Jan. 2; 284(1):478-85). In some cases, an NLS has the formula: (K/R) (K/R) X₁₀₋₁₂(K/R)₃₋₅. In some cases, an NLS has the formula: K(K/R)×(K/R).

In some embodiments a cationic polypeptide of a cationic polypeptide composition includes one more (e.g., two or more, three or more, or four or more) NLSs. In some cases the cationic polypeptide is not a histone protein or histone fragment (e.g., is not an HTP). Thus, in some cases the cationic polypeptide of a cationic polypeptide composition is an NLS-containing peptide.

In some cases, the NLS-containing peptide includes a cysteine residue, which can facilitate conjugation to: a cationic (or in some cases anionic) amino acid polymer, a linker, histone protein for HTP, and/or other cationic polypeptides (e.g., in some cases as part of a branched histone structure). For example, a cysteine residue can be used for crosslinking (conjugation) via sulfhydryl chemistry (e.g., a disulfide bond) and/or amine-reactive chemistry. In some cases the cysteine residue is internal. In some cases the cysteine residue is positioned at the N-terminus and/or C-terminus. In some cases, an NLS-containing peptide of a cationic polypeptide composition includes a mutation (e.g., insertion or substitution) (e.g., relative to a wild type amino acid sequence) that adds a cysteine residue.

Examples of NLSs that can be used as an NLS-containing peptide (or conjugated to any convenient cationic polypeptide such as an HTP or cationic polymer or cationic amino acid polymer or anionic amino acid polymer) include but are not limited to (some of which include a cysteine residue):

(SEQ ID NO: 151)
PKKKRKV (T-ag NLS)
(SEQ ID NO: 152)
PKKKRKVEDPYC - SV40 T-Ag-derived NLS
(SEQ ID NO: 153)
PKKKRKVGPKKKRKVGPKKKRKVGPKKKRKVGC (NLS SV40)
(SEQ ID NO: 154)
CYGRKKRRQRRR - N-terminal cysteine of
cysteine-TAT
(SEQ ID NO: 155)
CSIPPEVKFNKPFVYLI
(SEQ ID NO: 156)
DRQIKIWFQNRRMKWKK
(SEQ ID NO: 157)
PKKKRKVEDPYC - C-term cysteine of an SV40
T-Ag-derived NLS
(SEQ ID NO: 158)
PAAKRVKLD [cMyc NLS]

For non-limiting examples of NLSs that can be used, see, e.g., Kosugi et al., J Biol Chem. 2009 Jan. 2; 284(1):478-85, e.g., see FIG. 5 of this disclosure.

Mitochondrial Localization Signal

In some embodiments a cationic polypeptide (e.g., a histone or HTP, e.g., H1, H2, H2A, H2AX, H2B, H3, or H4), an anionic polymer, and/or a cationic polymer of a subject nanoparticle includes (and/or is conjugated to) one or more (e.g., two or more, three or more, or four or more) mitochondrial localization sequences. Any convenient mitochondrial localization sequence can be used. Examples of mitochondrial localization sequences include but are not limited to: PEDEIWLPEPESVDVPAKPISTSSMMMP (SEQ ID NO: 149), a mitochondrial localization sequence of SDHB, mono/di/triphenylphosphonium or other phosphoniums, VAMP 1A, VAMP 1B, the 67 N-terminal amino acids of DGAT2, and the 20 N-terminal amino acids of Bax.

Sheddable Layer (Sheddable Coat)—e.g., of a Nanoparticle

In some embodiments, a subject nanoparticle includes a sheddable layer (also referred to herein as a “transient stabilizing layer”) that surrounds (encapsulates) the core. In some cases a subject sheddable layer can protect the payload before and during initial cellular uptake. For example, without a sheddable layer, much of the payload can be lost during cellular internalization. Once in the cellular environment, a sheddable layer ‘sheds’ (e.g., the layer can be pH- and/or or glutathione-sensitive), exposing the components of the core.

In some cases a subject sheddable layer includes silica. In some cases, when a subject nanoparticle includes a sheddable layer (e.g., of silica), greater intracellular delivery efficiency can be observed despite decreased probability of cellular uptake. Without wishing to be bound by any particular theory, coating a nanoparticle core with a sheddable layer (e.g., silica coating) can seal the core, stabilizing it until shedding of the layer, which leads to release of the payload (e.g., upon processing in the intended subcellular compartment). Following cellular entry through receptor-mediated endocytosis, the nanoparticle sheds its outermost layer, the sheddable layer degrades in the acidifying environment of the endosome or reductive environment of the cytosol, and exposes the core, which in some cases exposes localization signals such as nuclear localization signals (NLSs) and/or mitochondrial localization signals. Moreover, nanoparticle cores encapsulated by a sheddable layer can be stable in serum and can be suitable for administration in vivo.

Any desired sheddable layer can be used, and one of ordinary skill in the art can take into account where in the target cell (e.g., under what conditions, such as low pH) they desire the payload to be released (e.g., endosome, cytosol, nucleus, lysosome, and the like). Different sheddable layers may be more desirable depending on when, where, and/or under what conditions it would be desirable for the sheddable coat to shed (and therefore release the payload). For example, a sheddable layer can be acid labile. In some cases the sheddable layer is an anionic sheddable layer (an anionic coat). In some cases the sheddable layer comprises silica, a peptoid, a polycysteine, and/or a ceramic (e.g., a bioceramic). In some cases the sheddable includes one or more of: calcium, manganese, magnesium, iron (e.g., the sheddable layer can be magnetic, e.g., Fe₃MnO₂), and lithium. Each of these can include phosphate or sulfate. As such, in some cases the sheddable includes one or more of: calcium phosphate, calcium sulfate, manganese phosphate, manganese sulfate, magnesium phosphate, magnesium sulfate, iron phosphate, iron sulfate, lithium phosphate, and lithium sulfate; each of which can have a particular effect on how and/or under which conditions the sheddable layer will ‘shed.’ Thus, in some cases the sheddable layer includes one or more of: silica, a peptoid, a polycysteine, a ceramic (e.g., a bioceramic), calcium, calcium phosphate, calcium sulfate, calcium oxide, hydroxyapatite, manganese, manganese phosphate, manganese sulfate, manganese oxide, magnesium, magnesium phosphate, magnesium sulfate, magnesium oxide, iron, iron phosphate, iron sulfate, iron oxide, lithium, lithium phosphate, and lithium sulfate (in any combination thereof) (e.g., the sheddable layer can be a coating of silica, peptoid, polycysteine, a ceramic (e.g., a bioceramic), calcium phosphate, calcium sulfate, manganese phosphate, manganese sulfate, magnesium phosphate, magnesium sulfate, iron phosphate, iron sulfate, lithium phosphate, lithium sulfate, or a combination thereof). In some cases the sheddable layer includes silica (e.g., the sheddable layer can be a silica coat). In some cases the sheddable layer includes an alginate gel. For example a sheddable layer can in some cases be composed of biocompatible ceramic, organic or biopolymer functionalized ceramic, anionic polypeptides, or cationic polypeptides.

A sheddable layer may include peptide domains that promote endosomal escape or organelle localization such as nuclear localization signals. Additionally, Cathepsin-cleavable and MMP-cleavable domains may be included to promote accumulation and subsequent activity within specific cellular and tissue environments.

In some cases different release times for different payloads are desirable. For example, in some cases it is desirable to release a payload early (e.g., within 0.5-7 days of contacting a target cell) and in some cases it is desirable to release a payload late (e.g., within 6 days-30 days of contacting a target cell). For example, in some cases it may be desirable to release a payload (e.g., a gene editing tool such as a CRISPR/Cas guide RNA, a DNA molecule encoding said CRISPR/Cas guide RNA, a CRISPR/Cas RNA-guided polypeptide, and/or a nucleic acid molecule encoding said CRISPR/Cas RNA-guided polypeptide) within 0.5-7 days of contacting a target cell (e.g., within 0.5-5 days, 0.5-3 days, 1-7 days, 1-5 days, or 1-3 days of contacting a target cell). In some cases it may be desirable to release a payload (e.g., a Donor DNA molecule) within 6-40 days of contacting a target cell (e.g., within 6-30, 6-20, 6-15, 7-40, 7-30, 7-20, 7-15, 9-40, 9-30, 9-20, or 9-15 days of contacting a target cell). In some cases release times can be controlled by delivering nanoparticles having different payloads at different times. In some cases release times can be controlled by delivering nanoparticles at the same time (as part of different formulations or as part of the same formulation), where the components of the nanoparticle are designed to achieve the desired release times. For example, one may use a sheddable layer that degrades faster or slower, core components that are more or less resistant to degradation, core components that are more or less susceptible to de-condensation, etc. —and any or all of the components can be selected in any convenient combination to achieve the desired timing.

In some cases it is desirable to delay the release of a payload (e.g., a Donor DNA molecule) relative to another payload (e.g., one or more gene editing tools). As an example, in some cases a first nanoparticle includes a donor DNA molecule as a payload is designed such that the payload is released within 6-40 days of contacting a target cell (e.g., within 6-30, 6-20, 6-15, 7-40, 7-30, 7-20, 7-15, 9-40, 9-30, 9-20, or 9-15 days of contacting a target cell), while a second nanoparticle that includes one or more gene editing tools (e.g., a ZFP or nucleic acid encoding the ZFP, a TALE or a nucleic acid encoding the TALE, a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like) as a payload is designed such that the payload is released within 0.5-7 days of contacting a target cell (e.g., within 0.5-5 days, 0.5-3 days, 1-7 days, 1-5 days, or 1-3 days of contacting a target cell). The second nanoparticle can be part of the same or part of a different formulation as the first nanoparticle.

In some cases, a nanoparticle includes more than one payload, where it is desirable for the payloads to be released at different times. This can be achieved in a number of different ways. For example, a nanoparticle can have more than one core, where one core is made with components that can release the payload early (e.g., within 0.5-7 days of contacting a target cell, e.g., within 0.5-5 days, 0.5-3 days, 1-7 days, 1-5 days, or 1-3 days of contacting a target cell) (e.g., an siRNA, an mRNA, and/or a genome editing tool such as a ZFP or nucleic acid encoding the ZFP, a TALE or a nucleic acid encoding the TALE, a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like) and the other is made with components that can release the payload (e.g., a Donor DNA molecule) later (e.g., within 6-40 days of contacting a target cell, e.g., within 6-30, 6-20, 6-15, 7-40, 7-30, 7-20, 7-15, 9-40, 9-30, 9-20, or 9-15 days of contacting a target cell).

As another example, a nanoparticle can include more than one sheddable layer, where the outer sheddable layer is shed (releasing a payload) prior to an inner sheddable layer being shed (releasing another payload). In some cases, the inner payload is a Donor DNA molecule and the outer payload is one or more gene editing tools (e.g., a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like). The inner and outer payloads can be any desired payload and either or both can include, for example, one or more siRNAs and/or one or more mRNAs. As such, in some cases a nanoparticle can have more than one sheddable layer and can be designed to release one payload early (e.g., within 0.5-7 days of contacting a target cell, e.g., within 0.5-5 days, 0.5-3 days, 1-7 days, 1-5 days, or 1-3 days of contacting a target cell) (e.g., an siRNA, an mRNA, a genome editing tool such as a ZFP or nucleic acid encoding the ZFP, a TALE or a nucleic acid encoding the TALE, a ZFN or nucleic acid encoding the ZFN, a TALEN or a nucleic acid encoding the TALEN, a CRISPR/Cas guide RNA or DNA molecule encoding the CRISPR/Cas guide RNA, a CRISPR/Cas RNA-guided polypeptide or a nucleic acid molecule encoding the CRISPR/Cas RNA-guided polypeptide, and the like), and another payload (e.g., an siRNA, an mRNA, a Donor DNA molecule) later (e.g., within 6-40 days of contacting a target cell, e.g., within 6-30, 6-20, 6-15, 7-40, 7-30, 7-20, 7-15, 9-40, 9-30, 9-20, or 9-15 days of contacting a target cell).

In some embodiments (e.g., in embodiments described above), time of altered gene expression can be used as a proxy for the time of payload release. As an illustrative example, if one desires to determine if a payload has been released by day 12, one can assay for the desired result of nanoparticle delivery on day 12. For example, if the desired result was to reduce the expression of a target gene of the target cell, e.g., by delivering an siRNA, then the expression of the target gene can be assayed/monitored to determine if the siRNA has been released. As another example, if the desired result was to express a protein of interest, e.g., by delivering a DNA or mRNA encoding the protein of interest, then the expression of the protein of interest can be assayed/monitored to determine if the payload has been released. As yet another example, if the desired result was to alter the genome of the target cell, e.g., via cleaving genomic DNA and/or inserting a sequence of a donor DNA molecule, the expression from the targeted locus and/or the presence of genomic alterations can be assayed/monitored to determine if the payload has been released.

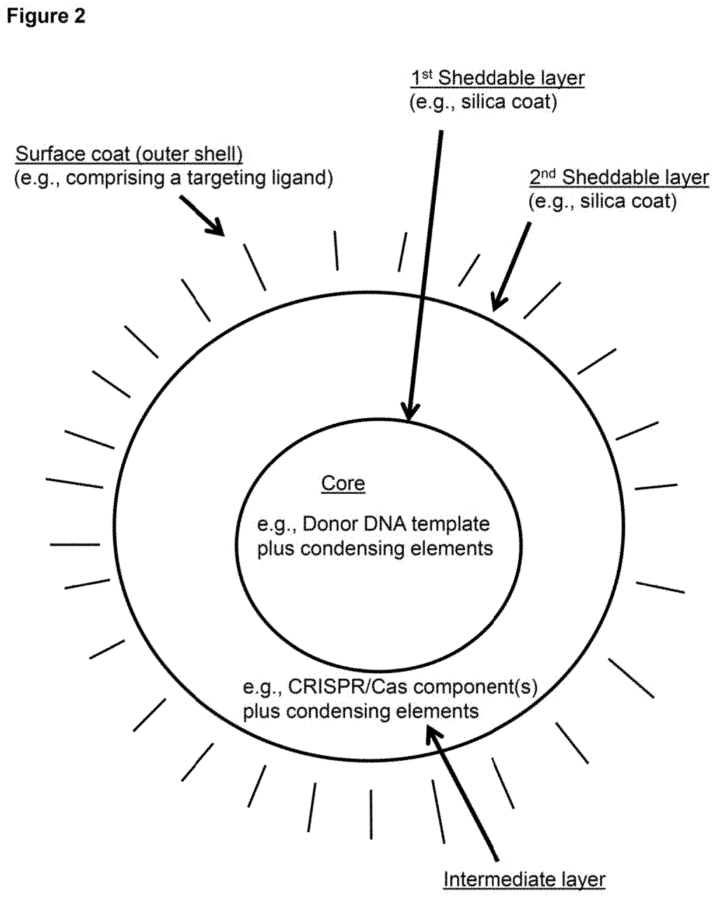

As such, in some cases a sheddable layer provides for a staged release of nanoparticle components. For example, in some cases, a nanoparticle has more than one (e.g., two, three, or four) sheddable layers. For example, for a nanoparticle with two sheddable layers, such a nanoparticle can have, from inner-most to outer-most: a core, e.g., with a first payload; a first sheddable layer, an intermediate layer e.g., with a second payload; and a second sheddable layer surrounding the intermediate layer (see, e.g., FIG. 2). Such a configuration (multiple sheddable layers) facilitates staged release of various desired payloads. As a further illustrative example, a nanoparticle with two sheddable layers (as described above) can include one or more desired gene editing tools in the core (e.g., one or more of: a Donor DNA molecule, a CRISPR/Cas guide RNA, a DNA encoding a CRISPR/Cas guide RNA, and the like), and another desired gene editing tool in the intermediate layer (e.g., one or more of: a programmable gene editing protein such as a CRISPR/Cas protein, a ZFP, a ZFN, a TALE, a TALEN, etc.; a DNA or RNA encoding a programmable gene editing protein; a CRISPR/Cas guide RNA; a DNA encoding a CRISPR/Cas guide RNA; and the like)—in any desired combination.

Surface Coat (Outer Shell) of a Nanoparticle

In some cases, the sheddable layer (the coat), is itself coated by an additional layer, referred to herein as an “outer shell,” “outer coat,” or “surface coat.” A surface coat can serve multiple different functions. For example, a surface coat can increase delivery efficiency and/or can target a subject nanoparticle to a particular cell type. The surface coat can include a peptide, a polymer, or a ligand-polymer conjugate. The surface coat can include a targeting ligand. The surface coat may be a layer upon a substrate (e.g. nanoparticle with electrostatic surface) or may contain its own conjugation or electrostatic condensation domains that independently present a ligand on the surface of a nanoparticle (see click chemistry and electrostatic approaches detailed elsewhere). For example, an aqueous solution of one or more targeting ligands (with or without linker domains) can be added to a coated nanoparticle suspension (suspension of nanoparticles coated with a sheddable layer). For example, in some cases the final concentration of protonated anchoring residues (of an anchoring domain) is between 25 and 300 μM. In some cases, the process of adding the surface coat yields a monodispersed suspension of particles with a mean particle size between 50 and 150 nm and a zeta potential between 0 and −10 mV.

In some cases the surface coat includes a targeting ligand (described in more detail elsewhere herein). In some cases the surface coat includes a stealth motif. A stealth motif is a motif that renders an entity (e.g., a pathogen, a nanoparticle, etc.) invisible a host immune system. Examples of stealth motifs include but are not limited to: polysialic acid, sialic acid and/or neuraminic acid functionalized peptides, hyaluronan, other anionic polypeptide/peptoid/polymer sequences, other glycoprotein modifications, brushed glycoproteins and anionic branches, native human-derived peptide sequences or sequences not found in databases of immunogenicity, and polyethylene glycol [see, e.g., Deepagan et al, J Nanosci Nanotechnol. 2013 Nov.; 13(11):7312-8; Sperisen et al., PLoS Comput Biol. 2005 November; 1(6):e6; and Yu et al., J Control Release. 2016 Oct. 28; 240:24-37]

In some cases, the surface coat interacts electrostatically with the outermost sheddable layer. For example, in some cases, a nanoparticle has two sheddable layers (e.g., from inner-most to outer-most: a core, e.g., with a first payload; a first sheddable layer, an intermediate layer e.g., with a second payload; and a second sheddable layer surrounding the intermediate layer), and the outer shell (surface coat) can interact with (e.g., electrostatically) the second sheddable layer. In some cases, a nanoparticle has only one sheddable layer (e.g., an anionic silica layer), and the outer shell can in some cases electrostatically interact with the sheddable layer.

Thus, in cases where the sheddable layer (e.g., outermost sheddable layer) is anionic (e.g., in some cases where the sheddable layer is a silica coat), the surface coat can interact electrostatically with the sheddable layer if the surface coat includes a cationic component. For example, in some cases the surface coat includes a delivery molecule in which a targeting ligand is conjugated to a cationic anchoring domain. The cationic anchoring domain interacts electrostatically with the sheddable layer and anchors the delivery molecule to the nanoparticle. Likewise, in cases where the sheddable layer (e.g., outermost sheddable layer) is cationic, the surface coat can interact electrostatically with the sheddable layer if the surface coat includes an anionic component.

In some embodiments, the surface coat includes a cell penetrating peptide (CPP). In some cases, a polymer of a cationic amino acid can function as a CPP (also referred to as a ‘protein transduction domain’-PTD), which is a term used to refer to a polypeptide, polynucleotide, carbohydrate, or organic or inorganic compound that facilitates traversing a lipid bilayer, micelle, cell membrane, organelle membrane, or vesicle membrane. A PTD attached to another molecule (e.g., embedded in and/or interacting with a sheddable layer of a subject nanoparticle), which can range from a small polar molecule to a large macromolecule and/or a nanoparticle, facilitates the molecule traversing a membrane, for example going from extracellular space to intracellular space, or cytosol to within an organelle (e.g., the nucleus).

Examples of CPPs include but are not limited to a minimal undecapeptide protein transduction domain (corresponding to residues 47-57 of HIV-1 TAT comprising YGRKKRRQRRR (SEQ ID NO: 160); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 161); Transportan GWTLNSAGYLLGKINLKALAALAKKIL (SEQ ID NO: 162); KALAWEAKLAKALAKALAKHLAKALAKALKCEA (SEQ ID NO: 163); and RQIKIWFQNRRMKWKK (SEQ ID NO: 164). Example CPPs include but are not limited to: YGRKKRRQRRR (SEQ ID NO: 160), RKKRRQRRR (SEQ ID NO: 165), an arginine homopolymer of from 3 arginine residues to 50 arginine residues, RKKRRQRR (SEQ ID NO: 166), YARAAARQARA (SEQ ID NO: 167), THRLPRRRRRR (SEQ ID NO: 168), and GGRRARRRRRR (SEQ ID NO: 169). In some embodiments, the CPP is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane

In some cases a CPP can be added to the nanoparticle by contacting a coated core (a core that is surrounded by a sheddable layer) with a composition (e.g., solution) that includes the CPP. The CPP can then interact with the sheddable layer (e.g., electrostatically).

In some cases, the surface coat includes a polymer of a cationic amino acid (e.g., a poly(arginine) such as poly(L-arginine) and/or poly(D-arginine), a poly(lysine) such as poly(L-lysine) and/or poly(D-lysine), a poly(histidine) such as poly(L-histidine) and/or poly(D-histidine), a poly(ornithine) such as poly(L-ornithine) and/or poly(D-ornithine), poly(citrulline) such as poly(L-citrulline) and/or poly(D-citrulline), and the like). As such, in some cases the surface coat includes poly(arginine), e.g., poly(L-arginine).

In some embodiments, the surface coat includes a heptapeptide such as selank (TKPRPGP—SEQ ID NO: 147) (e.g., N-acetyl selank) and/or semax (MEHFPGP—SEQ ID NO: 148) (e.g., N-acetyl semax). As such, in some cases the surface coat includes selank (e.g., N-acetyl selank). In some cases the surface coat includes semax (e.g., N-acetyl semax).

In some embodiments the surface coat includes a delivery molecule. A delivery molecule includes a targeting ligand and in some cases the targeting ligand is conjugated to an anchoring domain (e.g. a cationic anchoring domain or anionic anchoring domain). In some cases a targeting ligand is conjugated to an anchoring domain (e.g. a cationic anchoring domain or anionic anchoring domain) via an intervening linker.

### Multivalent Surface Coat

In some cases the surface coat includes any one or more of (in any desired combination): (i) one or more of the above described polymers, (ii) one or more targeting ligands, one or more CPPs, and one or more heptapeptides. For example, in some cases a surface coat can include one or more (e.g., two or more, three or more) targeting ligands, but can also include one or more of the above described cationic polymers. In some cases a surface coat can include one or more (e.g., two or more, three or more) targeting ligands, but can also include one or more CPPs. Further, a surface coat may include any combination of glycopeptides to promote stealth functionality, that is, to prevent serum protein adsorption and complement activity. This may be accomplished through Azide-alkyne click chemistry, coupling a peptide containing propargyl modified residues to azide containing derivatives of sialic acid, neuraminic acid, and the like.

In some cases, a surface coat includes a combination of targeting ligands that provides for targeted binding to CD34 and heparin sulfate proteoglycans. For example, poly(L-arginine) can be used as part of a surface coat to provide for targeted binding to heparin sulfate proteoglycans. As such, in some cases, after surface coating a nanoparticle with a cationic polymer (e.g., poly(L-arginine)), the coated nanoparticle is incubated with hyaluronic acid, thereby forming a zwitterionic and multivalent surface.

In some embodiments, the surface coat is multivalent. A multivalent surface coat is one that includes two or more targeting ligands (e.g., two or more delivery molecules that include different ligands). An example of a multimeric (in this case trimeric) surface coat (outer shell) is one that includes the targeting ligands stem cell factor (SCF) (which targets c-Kit receptor, also known as CD117), CD70 (which targets CD27), and SH2 domain-containing protein 1A (SH2D1A) (which targets CD150). For example, in some cases, to target hematopoietic stem cells (HSCs) [KLS (c-Kit⁺Lin⁻ Sca-1⁺) and CD27⁺/IL-7Ra⁻/CD150⁺/CD34⁺], a subject nanoparticle includes a surface coat that includes a combination of the targeting ligands SCF, CD70, and SH2 domain-containing protein 1A (SH2D1A), which target c-Kit, CD27, and CD150, respectively (see, e.g., Table 1). In some cases, such a surface coat can selectively target HSPCs and long-term HSCs (c-Kit+/Lin-/Sca-1+/CD27+/IL-7Ra-/CD150+/CD34-) over other lymphoid and myeloid progenitors. Other HSC lineages may be targeted in human, mouse, or other animal model cell population subsets using transcriptomics and proteomics data through a diagnostically-responsive ligand panel, e.g. ligands corresponding to overexpressed receptors in htt followed by ps followed by //ww follwed by w.ncbi.nlm followed by .nih.go followed by v/pmc/articles/PMC5305050/, and ht followed by tps followed by ://ww followed by w.nature.c followed by om/articles/s41421-018-0038-x. In some example embodiments, all three targeting ligands (SCF, CD70, and SH2D1A) are anchored to the nanoparticle via fusion to a cationic anchoring domain (e.g., a poly-histidine such as 6H, a poly-arginine such as 9R, and the like). For example, (1) the targeting polypeptide SCF (which targets c-Kit receptor) can include XMEGICRNRVTNNVKDVTKLVANLPKDYMITLKYVPGMDVLPSHCWISEMVVQLSDSLTDLLDKF SNISEGLSNYSIIDKLVNIVDDLVECVKENSSKDLKKSFKSPEPRLFTPEEFFRIFNRSIDAFKDFVVAS ETSDCVVSSTLSPEKDSRVSVTKPFMLPPVAX (SEQ ID NO: 194), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence; (2) the targeting polypeptide CD70 (which targets CD27) can include XPEEGSGCSVRRRPYGCVLRAALVPLVAGLVICLVVCIQRFAQAQQQLPLESLGWDVAELQLNHTG PQQDPRLYWQGGPALGRSFLHGPELDKGQLRIHRDGIYMVHIQVTLAICSSTTASRHHPTTLAVGIC SPASRSISLLRLSFHQGCTIASQRLTPLARGDTLCTNLTGTLLPSRNTDETFFGVQWVRPX (SEQ ID NO: 195), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence; and (3) the targeting polypeptide SH2D1A (which targets CD150) can include XSSGLVPRGSHMDAVAVYHGKISRETGEKLLLATGLDGSYLLRDSESVPGVYCLCVLYHGYIYTYR VSQTETGSWSAETAPGVHKRYFRKIKNLISAFQKPDQGIVIPLQYPVEKKSSARSTQGTTGIREDPDVC LKAP (SEQ ID NO: 196), where the X is a cationic anchoring domain (e.g., a poly-histidine such as 6H, a poly-arginine such as 9R, and the like), e.g., which can in some cases be present at the N- and/or C-terminal end, or can be embedded within the polypeptide sequence (e.g., such as MGSSXSSGLVPRGSHMDAVAVYHGKISRETGEKLLLATGLDGSYLLRDSESVPGVYCLCVLYHGYIY TYRVSQTETGSWSAETAPGVHKRYFRKIKNLISAFQKPDQGIVIPLQYPVEKKSSARSTQGTTGIRED PDVCLKAP (SEQ ID NO: 197)).

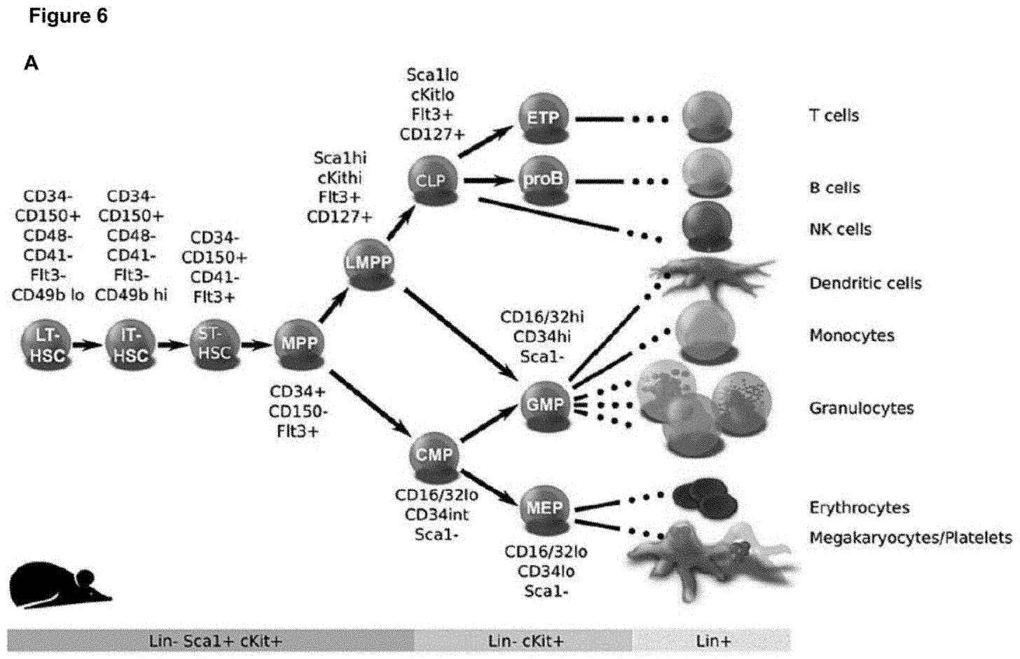

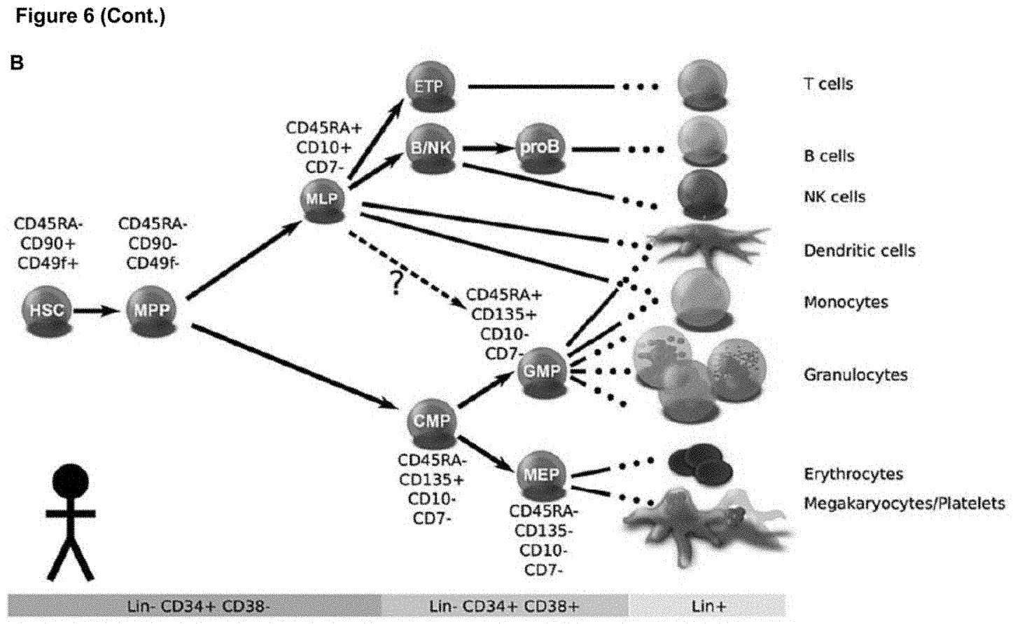

As noted above, nanoparticles of the disclosure can include multiple targeting ligands (as part of a surface coat) in order to target a desired cell type, or in order to target a desired combination of cell types. Examples of cells of interest within the mouse and human hematopoietic cell lineages are depicted in FIG. 6 (panels A-B), along with markers that have been identified for those cells. For example, various combinations of cell surface markers of interest include, but are not limited to: [Mouse] (i) CD150; (ii) Sca1, cKit, CD150; (iii) CD150 and CD49b; (iv) Sca1, cKit, CD150, and CD49b; (v) CD150 and Flt3; (vi) Sca1, cKit, CD150, and Flt3; (vii) Flt3 and CD34; (viii) Flt3, CD34, Sca1, and cKit; (ix) Flt3 and CD127; (x) Sca1, cKit, Flt3, and CD127; (xi) CD34; (xii) cKit and CD34; (xiii) CD16/32 and CD34; (xiv) cKit, CD16/32, and CD34; and (xv) cKit; and [Human] (i) CD90 and CD49f; (ii) CD34, CD90, and CD49f; (iii) CD34; (iv) CD45RA and CD10; (v) CD34, CD45RA, and CD10; (vi) CD45RA and CD135; (vii) CD34, CD38, CD45RA, and CD135; (viii) CD135; (ix) CD34, CD38, and CD135; and (x) CD34 and CD38. Thus, in some cases a surface coat includes one or more targeting ligands that provide targeted binding to a surface protein or combination of surface proteins selected from: [Mouse] (i) CD150; (ii) Sca1, cKit, CD150; (iii) CD150 and CD49b; (iv) Sca1, cKit, CD150, and CD49b; (v) CD150 and Flt3; (vi) Sca1, cKit, CD150, and Flt3; (vii) Flt3 and CD34; (viii) Flt3, CD34, Sca1, and cKit; (ix) Flt3 and CD127; (x) Sca1, cKit, Flt3, and CD127; (xi) CD34; (xii) cKit and CD34; (xiii) CD16/32 and CD34; (xiv) cKit, CD16/32, and CD34; and (xv) cKit; and [Human] (i) CD90 and CD49f; (ii) CD34, CD90, and CD49f; (iii) CD34; (iv) CD45RA and CD10; (v) CD34, CD45RA, and CD10; (vi) CD45RA and CD135; (vii) CD34, CD38, CD45RA, and CD135; (viii) CD135; (ix) CD34, CD38, and CD135; and (x) CD34 and CD38. Because a subject nanoparticle can include more than one targeting ligand, and because some cells include overlapping markers, multiple different cell types can be targeted using combinations of surface coats, e.g., in some cases a surface coat may target one specific cell type while in other cases a surface coat may target more than one specific cell type (e.g., 2 or more, 3 or more, 4 or more cell types). A variety of other targeting ligands may be used as determined diagnostically-responsively through cell specificity, tissue specificity, and organ specificity indices vs. other cells (e.g. proteomics/transcriptomics data of whole blood, immune subpopulations), tissues (e.g. proteomics/transcriptomics data of specific subsets of cells in an organ), and organs (e.g. proteomics/transcriptomics data of the whole organ set of a biodistribution). In autologous or allogeneic cell contexts, where cells are optionally pre-enriched for desired cell type or cell types through industry-standard techniques (e.g. FACS, specialized growth mediums and other selection techniques), a cell-specificity index may be utilized for targeting relevant cell subpopulations without concern for off-target tissue/organ targeting in a system biodistribution context. For example, any combination of cells within the hematopoietic lineage can be targeted. As an illustrative example, targeting CD34 (using a targeting ligand that provides for targeted binding to CD34) can lead to nanoparticle delivery of a payload to several different cells within the hematopoietic lineage (see, e.g., FIGS. 6A-B). In some embodiments, a diseased cell subpopulation (e.g. not only with cancer cells, but also with genetic diseases or other degenerative conditions) may have an altered cell surface proteome, thereby requiring a tailored ligand-targeting approach as described in the ligand design and synthesis detailed descriptions and diagnostically-responsive approaches herein. For example, a hematopoietic stem cell's associated progenitors and direct lineages) carrying sickle cell disease (e.g. E7V) or B-thalassemia mutations may have altered cell surface proteomics/transcriptomics, whereby ligands developed for a healthy cell population may not be optimized for administering a therapeutic modality to a patient, autologous/allogeneic cell/tissue/organ type, or model organism. The methods and uses herein detail numerous strategies for circumventing these errors in therapeutic development (in terms of attaining cell type affinity and specificity) and creating ultra-tailorable therapeutics with modular components/architectures and tunable cell specificity based on genomic, transcriptomic and/or proteomic analysis of target cell populations (“diagnostically-responsive medicine”).

### Delivery Molecules

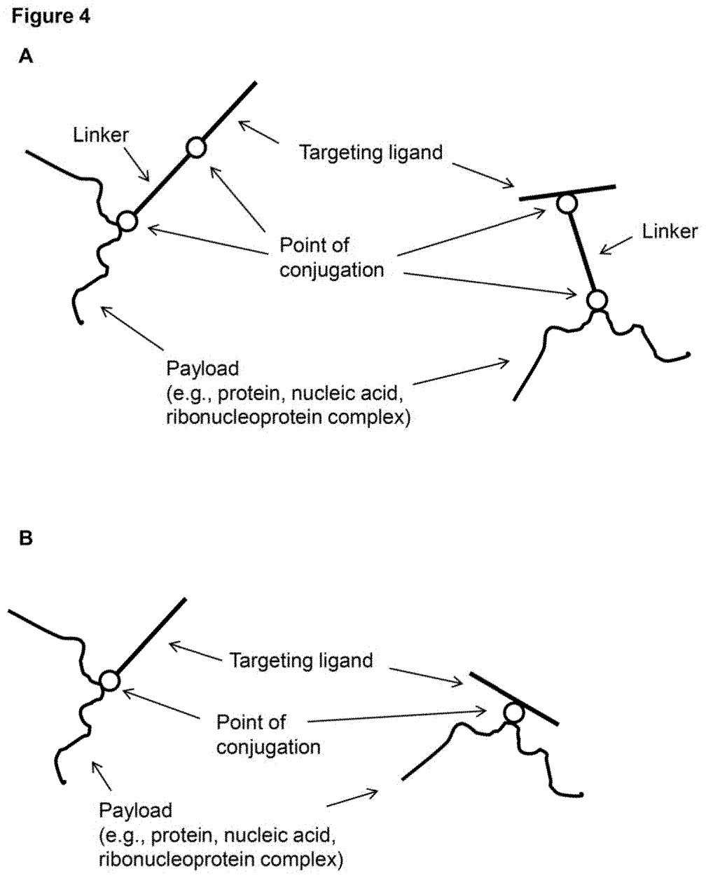

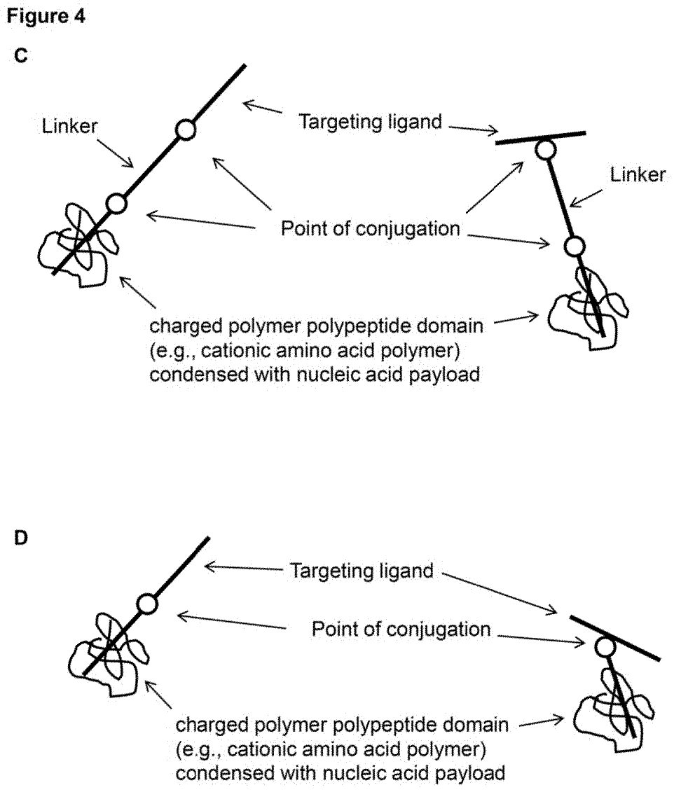

Provided are delivery molecules (a form of delivery vehicle) that include a targeting ligand (a peptide) conjugated to (i) a protein or nucleic acid payload, or (ii) a charged polymer polypeptide domain. The targeting ligand provides for (i) targeted binding to a cell surface protein, and in some cases (ii) engagement of a long endosomal recycling pathway. In some cases when the targeting ligand is conjugated to a charged polymer polypeptide domain, the charged polymer polypeptide domain interacts with (e.g., is condensed with) a nucleic acid payload and/or a protein payload. In some cases the targeting ligand is conjugated via an intervening linker. Refer to FIG. 4 for examples of different possible conjugation strategies (i.e., different possible arrangements of the components of a subject delivery molecule). In some cases, the targeting ligand provides for targeted binding to a cell surface protein, but does not necessarily provide for engagement of a long endosomal recycling pathway. Thus, also provided are delivery molecules that include a targeting ligand (e.g., peptide targeting ligand) conjugated to a protein or nucleic acid payload, or conjugated to a charged polymer polypeptide domain, where the targeting ligand provides for targeted binding to a cell surface protein (but does not necessarily provide for engagement of a long endosomal recycling pathway).

In some cases, the delivery molecules disclosed herein are designed such that a nucleic acid or protein payload reaches its extracellular target (e.g., by providing targeted biding to a cell surface protein) and is preferentially not destroyed within lysosomes or sequestered into ‘short’ endosomal recycling endosomes. Instead, delivery molecules of the disclosure can provide for engagement of the ‘long’ (indirect/slow) endosomal recycling pathway, which can allow for endosomal escape and/or or endosomal fusion with an organelle.

For example, in some cases, β-arrestin is engaged to mediate cleavage of seven-transmembrane GPCRs (McGovern et al., Handb Exp Pharmacol. 2014; 219:341-59; Goodman et al., Nature. 1996 Oct. 3; 383(6599):447-50; Zhang et al., J Biol Chem. 1997 Oct. 24; 272(43):27005-14) and/or single-transmembrane receptor tyrosine kinases (RTKs) from the actin cytoskeleton (e.g., during endocytosis), triggering the desired endosomal sorting pathway. Thus, in some embodiments the targeting ligand of a delivery molecule of the disclosure provides for engagement of β-arrestin upon binding to the cell surface protein (e.g., to provide for signaling bias and to promote internalization via endocytosis following orthosteric binding).

### Charged Polymer Polypeptide Domain

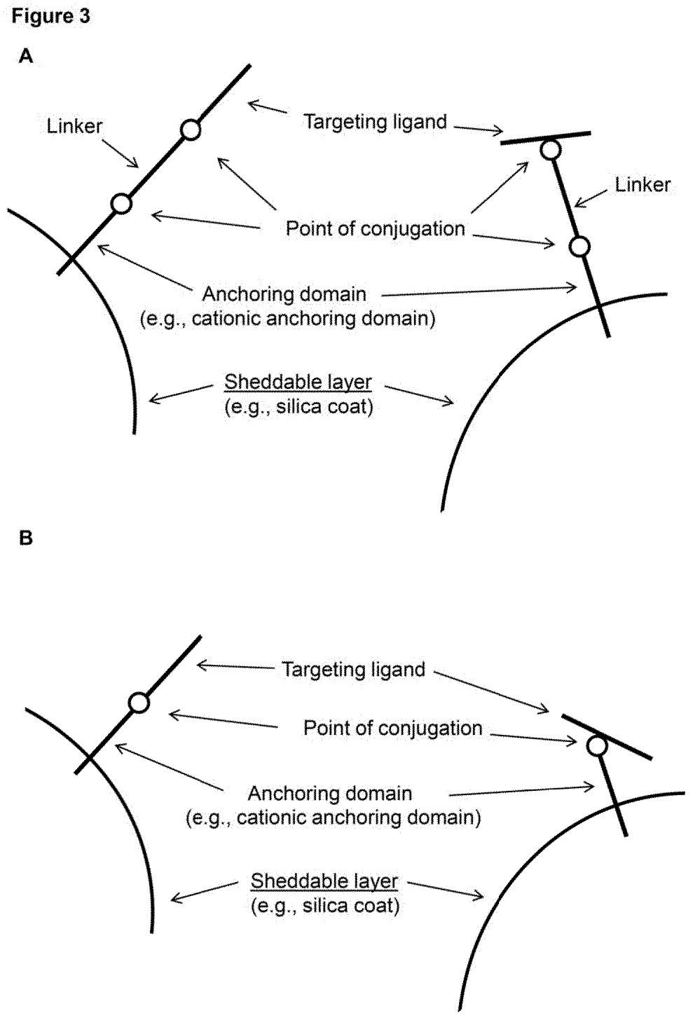

In some cases a targeting ligand (e.g., of a subject delivery molecule) is conjugated to a charged polymer polypeptide domain (an anchoring domain such as a cationic anchoring domain or an anionic anchoring domain) (see e.g., FIG. 3 and FIG. 4). Charged polymer polypeptide domains can include repeating residues (e.g., cationic residues such as arginine, lysine, histidine). In some cases, a charged polymer polypeptide domain (an anchoring domain) has a length in a range of from 3 to 30 amino acids (e.g., from 3-28, 3-25, 3-24, 3-20, 4-30, 4-28, 4-25, 4-24, or 4-20 amino acids; or e.g., from 4-15, 4-12, 5-30, 5-28, 5-25, 5-20, 5-15, 5-12 amino acids). In some cases, a charged polymer polypeptide domain (an anchoring domain) has a length in a range of from 4 to 24 amino acids. In some cases, a charged polymer polypeptide domain (an anchoring domain) has a length in a range of from 5 to 10 amino acids. Suitable examples of a charged polymer polypeptide domain include, but are not limited to: RRRRRRRRR (9R)(SEQ ID NO: 15) and HHHHHH (6H)(SEQ ID NO: 16).

A charged polymer polypeptide domain (a cationic anchoring domain, an anionic anchoring domain) can be any convenient charged domain (e.g., cationic charged domain). For example, such a domain can be a histone tail peptide (HTP) (described elsewhere herein in more detail). In some cases a charged polymer polypeptide domain includes a histone and/or histone tail peptide (e.g., a cationic polypeptide can be a histone and/or histone tail peptide). In some cases a charged polymer polypeptide domain includes an NLS-containing peptide (e.g., a cationic polypeptide can be an NLS-containing peptide). In some cases a charged polymer polypeptide domain includes a peptide that includes a mitochondrial localization signal (e.g., a cationic polypeptide can be a peptide that includes a mitochondrial localization signal).

In some cases, a charged polymer polypeptide domain of a subject delivery molecule is used as a way for the delivery molecular to interact with (e.g., interact electrostatically, e.g., for condensation) the payload (e.g., nucleic acid payload and/or protein payload).

In some cases, a charged polymer polypeptide domain of a subject delivery molecule is used as an anchor to coat the surface of a nanoparticle with the delivery molecule, e.g., so that the targeting ligand is used to target the nanoparticle to a desired cell/cell surface protein (see e.g., FIG. 3). Thus, in some cases, the charged polymer polypeptide domain interacts electrostatically with a charged stabilization layer of a nanoparticle. For example, in some cases a nanoparticle includes a core (e.g., including a nucleic acid, protein, and/or ribonucleoprotein complex payload) that is surrounded by a stabilization layer (e.g., a silica, peptoid, polycysteine, or calcium phosphate coating). In some cases, the stabilization layer has a negative charge and a positively charged polymer polypeptide domain can therefore interact with the stabilization layer (e.g., in some cases a sheddable layer), effectively anchoring the delivery molecule to the nanoparticle and coating the nanoparticle surface with a subject targeting ligand (see, e.g., FIG. 3). In some cases, the stabilization layer has a positive charge and a negatively charged polymer polypeptide domain can therefore interact with the stabilization layer, effectively anchoring the delivery molecule to the nanoparticle and coating the nanoparticle surface with a subject targeting ligand. Conjugation can be accomplished by any convenient technique and many different conjugation chemistries will be known to one of ordinary skill in the art. In some cases the conjugation is via sulfhydryl chemistry (e.g., a disulfide bond). In some cases the conjugation is accomplished using amine-reactive chemistry. In some cases, the targeting ligand and the charged polymer polypeptide domain are conjugated by virtue of being part of the same polypeptide.

In some cases a charged polymer polypeptide domain (cationic) can include a polymer selected from: poly(arginine)(PR), poly(lysine)(PK), poly(histidine)(PH), poly(ornithine), poly(citrulline), and a combination thereof. In some cases a given cationic amino acid polymer can include a mix of arginine, lysine, histidine, ornithine, and citrulline residues (in any convenient combination). Polymers can be present as a polymer of L-isomers or D-isomers, where D-isomers are more stable in a target cell because they take longer to degrade. Thus, inclusion of D-isomer poly(amino acids) delays degradation (and subsequent payload release). The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the release kinetics and enzymatic susceptibility to degradation and payload release.

In some cases a cationic polymer includes D-isomers and L-isomers of an cationic amino acid polymer (e.g., poly(arginine)(PR), poly(lysine)(PK), poly(histidine)(PH), poly(ornithine), poly(citrulline)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases a cationic polymer includes a first cationic polymer (e.g., amino acid polymer) that is a polymer of D-isomers (e.g., selected from poly(D-arginine), poly(D-lysine), poly(D-histidine), poly(D-ornithine), and poly(D-citrulline)); and includes a second cationic polymer (e.g., amino acid polymer) that is a polymer of L-isomers (e.g., selected from poly(L-arginine), poly(L-lysine), poly(L-histidine), poly(L-ornithine), and poly(L-citrulline)). In some cases the ratio of the first cationic polymer (D-isomers) to the second cationic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, a cationic polymer includes (e.g., in addition to or in place of any of the foregoing examples of cationic polymers) poly(ethylenimine), poly(amidoamine) (PAMAM), poly(aspartamide), polypeptoids (e.g., for forming “spiderweb”-like branches for core condensation), a charge-functionalized polyester, a cationic polysaccharide, an acetylated amino sugar, chitosan, or a cationic polymer that includes any combination thereof (e.g., in linear or branched forms).

In some embodiments, an cationic polymer can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases a cationic polymer includes poly(L-arginine), e.g., with a molecular weight of approximately 29 kDa. As another example, in some cases a cationic polymer includes linear poly(ethylenimine) with a molecular weight of approximately 25 kDa (PEI). As another example, in some cases a cationic polymer includes branched poly(ethylenimine) with a molecular weight of approximately 10 kDa. As another example, in some cases a cationic polymer includes branched poly(ethylenimine) with a molecular weight of approximately 70 kDa. In some cases a cationic polymer includes PAMAM.

In some cases, a cationic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) of a cationic polymer composition includes a cysteine residue. In some cases the cationic amino acid polymer includes cysteine residue on the N- and/or C-terminus. In some cases the cationic amino acid polymer includes an internal cysteine residue.

In some cases, a cationic amino acid polymer includes (and/or is conjugated to) a nuclear localization signal (NLS) (described in more detail below). Thus, in some embodiments a cationic amino acid polymer (e.g., poly(arginine)(PR), poly(lysine)(PK), poly(histidine)(PH), poly(ornithine), and poly(citrulline), poly(D-arginine)(PDR), poly(D-lysine)(PDK), poly(D-histidine)(PDH), poly(D-ornithine), and poly(D-citrulline), poly(L-arginine)(PLR), poly(L-lysine)(PLK), poly(L-histidine)(PLH), poly(L-ornithine), and poly(L-citrulline)) includes one or more (e.g., two or more, three or more, or four or more) NLSs. In some cases the cationic amino acid polymer includes an NLS on the N- and/or C-terminus. In some cases the cationic amino acid polymer includes an internal NLS.

In some cases, the charged polymer polypeptide domain is condensed with a nucleic acid payload and/or a protein payload (see e.g., FIG. 4). In some cases, the charged polymer polypeptide domain interacts electrostatically with a protein payload. In some cases, the charged polymer polypeptide domain is co-condensed with silica, salts, and/or anionic polymers to provide added endosomal buffering capacity, stability, and, e.g., optional timed release. In some cases, a charged polymer polypeptide domain of a subject delivery molecule is a stretch of repeating cationic residues (such as arginine, lysine, and/or histidine), e.g., in some 4-25 amino acids in length or 4-15 amino acids in length. Such a domain can allow the delivery molecule to interact electrostatically with an anionic sheddable matrix (e.g., a co-condensed anionic polymer). Thus, in some cases, a subject charged polymer polypeptide domain of a subject delivery molecule is a stretch of repeating cationic residues that interacts (e.g., electrostatically) with an anionic sheddable matrix and with a nucleic acid and/or protein payload. Thus, in some cases a subject delivery molecule interacts with a payload (e.g., nucleic acid and/or protein) and is present as part of a composition with an anionic polymer (e.g., co-condenses with the payload and with an anionic polymer).

The anionic polymer of an anionic sheddable matrix (i.e., the anionic polymer that interacts with the charged polymer polypeptide domain of a subject delivery molecule) can be any convenient anionic polymer/polymer composition. Examples include, but are not limited to: poly(glutamic acid) (e.g., poly(D-glutamic acid) (PDE), poly(L-glutamic acid) (PLE), both PDE and PLE in various desired ratios, etc.) In some cases, PDE is used as an anionic sheddable matrix. In some cases, PLE is used as an anionic sheddable matrix (anionic polymer). In some cases, PDE is used as an anionic sheddable matrix (anionic polymer). In some cases, PLE and PDE are both used as an anionic sheddable matrix (anionic polymer), e.g., in a 1:1 ratio (50% PDE, 50% PLE).

Anionic Polymer

An anionic polymer can include one or more anionic amino acid polymers. For example, in some cases a subject anionic polymer composition includes a polymer selected from: poly(glutamic acid)(PEA), poly(aspartic acid)(PDA), and a combination thereof. In some cases a given anionic amino acid polymer can include a mix of aspartic and glutamic acid residues. Each polymer can be present in the composition as a polymer of L-isomers or D-isomers, where D-isomers are more stable in a target cell because they take longer to degrade. Thus, inclusion of D-isomer poly(amino acids) can delay degradation and subsequent payload release. The payload release rate can therefore be controlled and is proportional to the ratio of polymers of D-isomers to polymers of L-isomers, where a higher ratio of D-isomer to L-isomer increases duration of payload release (i.e., decreases release rate). In other words, the relative amounts of D- and L-isomers can modulate the nanoparticle core's timed release kinetics and enzymatic susceptibility to degradation and payload release.

In some cases an anionic polymer composition includes polymers of D-isomers and polymers of L-isomers of an anionic amino acid polymer (e.g., poly(glutamic acid)(PEA) and poly(aspartic acid)(PDA)). In some cases the D- to L-isomer ratio is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1).

Thus, in some cases an anionic polymer composition includes a first anionic polymer (e.g., amino acid polymer) that is a polymer of D-isomers (e.g., selected from poly(D-glutamic acid) (PDEA) and poly(D-aspartic acid) (PDDA)); and includes a second anionic polymer (e.g., amino acid polymer) that is a polymer of L-isomers (e.g., selected from poly(L-glutamic acid) (PLEA) and poly(L-aspartic acid) (PLDA)). In some cases the ratio of the first anionic polymer (D-isomers) to the second anionic polymer (L-isomers) is in a range of from 10:1-1:10 (e.g., from 8:1-1:10, 6:1-1:10, 4:1-1:10, 3:1-1:10, 2:1-1:10, 1:1-1:10, 10:1-1:8, 8:1-1:8, 6:1-1:8, 4:1-1:8, 3:1-1:8, 2:1-1:8, 1:1-1:8, 10:1-1:6, 8:1-1:6, 6:1-1:6, 4:1-1:6, 3:1-1:6, 2:1-1:6, 1:1-1:6, 10:1-1:4, 8:1-1:4, 6:1-1:4, 4:1-1:4, 3:1-1:4, 2:1-1:4, 1:1-1:4, 10:1-1:3, 8:1-1:3, 6:1-1:3, 4:1-1:3, 3:1-1:3, 2:1-1:3, 1:1-1:3, 10:1-1:2, 8:1-1:2, 6:1-1:2, 4:1-1:2, 3:1-1:2, 2:1-1:2, 1:1-1:2, 10:1-1:1, 8:1-1:1, 6:1-1:1, 4:1-1:1, 3:1-1:1, or 2:1-1:1)

In some embodiments, an anionic polymer composition includes (e.g., in addition to or in place of any of the foregoing examples of anionic polymers) a glycosaminoglycan, a glycoprotein, a polysaccharide, poly(mannuronic acid), poly(guluronic acid), heparin, heparin sulfate, chondroitin, chondroitin sulfate, keratan, keratan sulfate, aggrecan, poly(glucosamine), or an anionic polymer that comprises any combination thereof.

In some embodiments, an anionic polymer can have a molecular weight in a range of from 1-200 kDa (e.g., from 1-150, 1-100, 1-50, 5-200, 5-150, 5-100, 5-50, 10-200, 10-150, 10-100, 10-50, 15-200, 15-150, 15-100, or 15-50 kDa). As an example, in some cases an anionic polymer includes poly(glutamic acid) with a molecular weight of approximately 15 kDa.

In some cases, an anionic amino acid polymer includes a cysteine residue, which can facilitate conjugation, e.g., to a linker, an NLS, and/or a cationic polypeptide (e.g., a histone or HTP). For example, a cysteine residue can be used for crosslinking (conjugation) via sulfhydryl chemistry (e.g., a disulfide bond) and/or amine-reactive chemistry. Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes a cysteine residue. In some cases the anionic amino acid polymer includes cysteine residue on the N- and/or C-terminus. In some cases the anionic amino acid polymer includes an internal cysteine residue.

In some cases, an anionic amino acid polymer includes (and/or is conjugated to) a nuclear localization signal (NLS) (described in more detail below). Thus, in some embodiments an anionic amino acid polymer (e.g., poly(glutamic acid) (PEA), poly(aspartic acid) (PDA), poly(D-glutamic acid) (PDEA), poly(D-aspartic acid) (PDDA), poly(L-glutamic acid) (PLEA), poly(L-aspartic acid) (PLDA)) of an anionic polymer composition includes (and/or is conjugated to) one or more (e.g., two or more, three or more, or four or more) NLSs. In some cases the anionic amino acid polymer includes an NLS on the N- and/or C-terminus. In some cases the anionic amino acid polymer includes an internal NLS.

In some cases, an anionic polymer is conjugated to a targeting ligand.

Linker In some embodiments a targeting ligand is conjugated to an anchoring domain (e.g., a cationic anchoring domain, an anionic anchoring domain) or to a payload via an intervening linker. The linker can be a protein linker or non-protein linker. A linker can in some cases aid in stability, prevent complement activation, and/or provide flexibility to the ligand relative to the anchoring domain.

Conjugation of a targeting ligand to a linker or a linker to an anchoring domain can be accomplished in a number of different ways. In some cases the conjugation is via sulfhydryl chemistry (e.g., a disulfide bond, e.g., between two cysteine residues). In some cases the conjugation is accomplished using amine-reactive chemistry. In some cases, a targeting ligand includes a cysteine residue and is conjugated to the linker via the cysteine residue; and/or an anchoring domain includes a cysteine residue and is conjugated to the linker via the cysteine residue. In some cases, the linker is a peptide linker and includes a cysteine residue. In some cases, the targeting ligand and a peptide linker are conjugated by virtue of being part of the same polypeptide; and/or the anchoring domain and a peptide linker are conjugated by virtue of being part of the same polypeptide.

In some cases, a subject linker is a polypeptide and can be referred to as a polypeptide linker. It is to be understood that while polypeptide linkers are contemplated, non-polypeptide linkers (chemical linkers) are used in some cases. For example, in some embodiments the linker is a polyethylene glycol (PEG) linker. Suitable protein linkers include polypeptides of between 4 amino acids and 60 amino acids in length (e.g., 4-50, 4-40, 4-30, 4-25, 4-20, 4-15, 4-10, 6-60, 6-50, 6-40, 6-30, 6-25, 6-20, 6-15, 6-10, 8-60, 8-50, 8-40, 8-30, 8-25, 8-20, or 8-15 amino acids in length).

In some embodiments, a subject linker is rigid (e.g., a linker that include one or more proline residues). One non-limiting example of a rigid linker is GAPGAPGAP (SEQ ID NO: 17). In some cases, a polypeptide linker includes a C residue at the N- or C-terminal end. Thus, in some case a rigid linker is selected from: GAPGAPGAPC (SEQ ID NO: 18) and CGAPGAPGAP (SEQ ID NO: 19).

Peptide linkers with a degree of flexibility can be used. Thus, in some cases, a subject linker is flexible. The linking peptides may have virtually any amino acid sequence, bearing in mind that flexible linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)ₙ, glycine-serine polymers (including, for example, (GS)ₙ, GSGGSₙ (SEQ ID NO: 20), GGSGGSₙ (SEQ ID NO: 21), and GGGSₙ (SEQ ID NO: 22), where n is an integer of at least one), glycine-alanine polymers, alanine-serine polymers. Example linkers can comprise amino acid sequences including, but not limited to, GGSG (SEQ ID NO: 23), GGSGG (SEQ ID NO: 24), GSGSG (SEQ ID NO: 25), GSGGG (SEQ ID NO: 26), GGGSG (SEQ ID NO: 27), GSSSG (SEQ ID NO: 28), and the like. The ordinarily skilled artisan will recognize that design of a peptide conjugated to any elements described above can include linkers that are all or partially flexible, such that the linker can include a flexible linker as well as one or more portions that confer less flexible structure. Additional examples of flexible linkers include, but are not limited to: GGGGGSGGGGG (SEQ ID NO: 29) and GGGGGSGGGGS (SEQ ID NO: 30). As noted above, in some cases, a polypeptide linker includes a C residue at the N- or C-terminal end. Thus, in some cases a flexible linker includes an amino acid sequence selected from: GGGGGSGGGGGC (SEQ ID NO: 31), CGGGGGSGGGGG (SEQ ID NO: 32), GGGGGSGGGGSC (SEQ ID NO: 33), and CGGGGGSGGGGS (SEQ ID NO: 34).

In some cases, a subject polypeptide linker is endosomolytic. Endosomolytic polypeptide linkers include but are not limited to: KALA (SEQ ID NO: 35) and GALA (SEQ ID NO: 36). As noted above, in some cases, a polypeptide linker includes a C residue at the N- or C-terminal end. Thus, in some cases a subject linker includes an amino acid sequence selected from: CKALA (SEQ ID NO: 37), KALAC (SEQ ID NO: 38), CGALA (SEQ ID NO: 39), and GALAC (SEQ ID NO: 40).

### Illustrative Examples of Sulfhydryl Coupling Reactions

(e.g., for conjugation via sulfhydryl chemistry, e.g., using a cysteine residue) (e.g., for conjugating a targeting ligand or glycopeptide to a linker, conjugating a targeting ligand or glycopeptide to an anchoring domain (e.g., cationic anchoring domain), conjugating a linker to an anchoring domain (e.g., cationic anchoring domain), and the like)

Disulfide Bond

Cysteine residues can form disulfide bonds under mild oxidizing conditions or at higher than neutral pH in aqueous conditions.

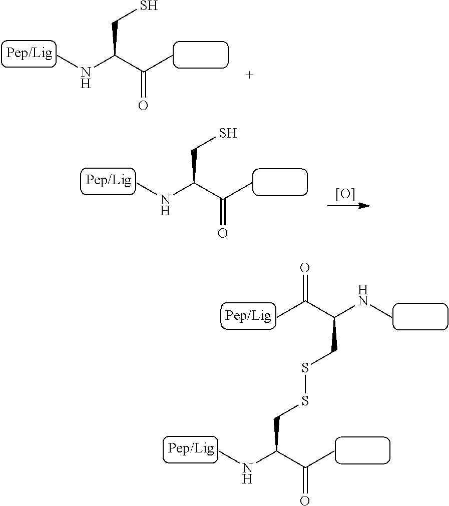

Thioether/Thioester Bond

Sulfhydryl groups of cysteine react with maleimide and acyl halide groups, forming stable thioether and thioester bonds respectively.

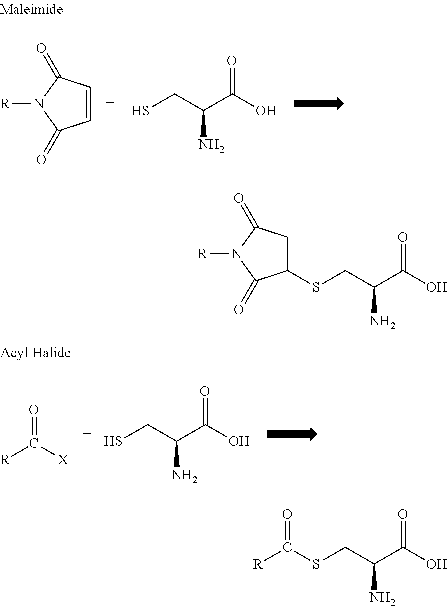

Azide—Alkyne Cycloaddition

This conjugation is facilitated by chemical modification of the cysteine residue to contain an alkyne bond, or by the use of an L-propargyl amino acid derivative (e.g., L-propargyl cysteine—pictured below) in synthetic peptide preparation (e.g., solid phase synthesis). Coupling is then achieved by means of Cu promoted click chemistry.

### Examples of Targeting Ligands

Examples of targeting ligands include, but are not limited to, those that include the following amino acid sequences:

SCF (targets/binds to c-Kit receptor)
(SEQ ID NO: 184)
EGICRNRVTNNVKDVTKLVANLPKDYMITLKYVPGMDVLPSHCWISEMVVQLSDSLTDLLDKFSNI
SEGLSNYSIIDKLVNIVDDLVECVKENSSKDLKKSFKSPEPRLFTPEEFFRIFNRSIDAFKDFVVASETS
DCVVSSTLSPEKDSRVSVTKPFMLPPVA;
CD70 (targets/binds to CD27)
(SEQ ID NO: 185)
PEEGSGCSVRRRPYGCVLRAALVPLVAGLVICLVVCIQRFAQAQQQLPLESLGWDVAELQLNHTGP
QQDPRLYWQGGPALGRSFLHGPELDKGQLRIHRDGIYMVHIQVTLAICSSTTASRHHPTTLAVGICS
PASRSISLLRLSFHQGCTIASQRLTPLARGDTLCTNLTGTLLPSRNTDETFFGVQWVRP;
and
SH2 domain-containing protein 1A (SH2D1A) (targets/binds to CD150)
(SEQ ID NO: 186)
SSGLVPRGSHMDAVAVYHGKISRETGEKLLLATGLDGSYLLRDSESVPGVYCLCVLYHGYIYTYRV
SQTETGSWSAETAPGVHKRYFRKIKNLISAFQKPDQGIVIPLQYPVEKKSSARSTQGTTGIREDPDVC
LKAP
Thus, non-limiting examples of targeting ligands (which can be used
alone or in combination with other targeting ligands) include:
9R-SCF
(SEQ ID NO: 189)
RRRRRRRRRMEGICRNRVTNNVKDVTKLVANLPKDYMITLKYVPGMDVLPSHCWISEMVVQLSD
SLTDLLDKFSNISEGLSNYSIIDKLVNIVDDLVECVKENSSKDLKKSFKSPEPRLFTPEEFFRIFNRSIDA
FKDFVVASETSDCVVSSTLSPEKDSRVSVTKPFMLPPVA
9R-CD70
(SEQ ID NO: 190)
RRRRRRRRRPEEGSGCSVRRRPYGCVLRAALVPLVAGLVICLVVCIQRFAQAQQQLPLESLGWDV
AELQLNHTGPQQDPRLYWQGGPALGRSFLHGPELDKGQLRIHRDGIYMVHIQVTLAICSSTTASRHH
PTTLAVGICSPASRSISLLRLSFHQGCTIASQRLTPLARGDTLCTNLTGTLLPSRNTDETFFGVQWVRP
CD70-9R
(SEQ ID NO: 191)
PEEGSGCSVRRRPYGCVLRAALVPLVAGLVICLVVCIQRFAQAQQQLPLESLGWDVAELQLNHTGP
QQDPRLYWQGGPALGRSFLHGPELDKGQLRIHRDGIYMVHIQVTLAICSSTTASRHHPTTLAVGICS
PASRSISLLRLSFHQGCTIASQRLTPLARGDTLCTNLTGTLLPSRNTDETFFGVQWVRPRRRRRRRR
R
6H-SH2D1A
(SEQ ID NO: 192)
MGSSHHHHHHSSGLVPRGSHMDAVAVYHGKISRETGEKLLLATGLDGSYLLRDSESVPGVYCLCVL
YHGYIYTYRVSQTETGSWSAETAPGVHKRYFRKIKNLISAFQKPDQGIVIPLQYPVEKKSSARSTQGT
TGIREDPDVCLKAP
6H-SH2D1A
(SEQ ID NO: 193)
RRRRRRRRRSSGLVPRGSHMDAVAVYHGKISRETGEKLLLATGLDGSYLLRDSESVPGVYCLCVLY
HGYIYTYRVSQTETGSWSAETAPGVHKRYFRKIKNLISAFQKPDQGIVIPLQYPVEKKSSARSTQGTT
GIREDPDVCLKAP
Illustrative examples of delivery molecules and components
(0a) Cysteine conjugation anchor 1 (CCA1)
[anchoring domain (e.g., cationic anchoring domain) - linker
(GAPGAPGAP) - cysteine]
(SEQ ID NO: 41)
RRRRRRRRR GAPGAPGAP C
(0b) Cysteine conjugation anchor 2 (CCA2)
[cysteine - linker (GAPGAPGAP) - anchoring domain (e.g., cationic
anchoring domain)]
(SEQ ID NO: 42)
C GAPGAPGAP RRRRRRRRR
(1a) α5β1 ligand
[anchoring domain (e.g., cationic anchoring domain) - linker
(GAPGAPGAP) - Targeting ligand]
(SEQ ID NO: 45)
RRRRRRRRR GAPGAPGAP RRETAWA
(1b) α5β1 ligand
[Targeting ligand - linker (GAPGAPGAP) - anchoring domain
(e.g., cationic anchoring domain)]
(SEQ ID NO: 46)
RRETAWA GAPGAPGAP RRRRRRRRR
(1c) α5β1 ligand - Cys left
(SEQ ID NO: 19)
CGAPGAPGAP
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(1d) α5β1 ligand - Cys right
(SEQ ID NO: 18)
GAPGAPGAPC
Note: This can be conjugated to CCA2 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(2a) RGD α5β1 ligand
[anchoring domain (e.g., cationic anchoring domain) - linker
(GAPGAPGAP) - Targeting ligand]
(SEQ ID NO: 47)
RRRRRRRRR GAPGAPGAP RGD
(2b) RGD α5b1 ligand
[Targeting ligand - linker (GAPGAPGAP) - anchoring domain
(e.g., cationic anchoring domain)]
(SEQ ID NO: 48)
RGD GAPGAPGAP RRRRRRRRR
(2c) RGD ligand - Cys left
(SEQ ID NO: 49)
CRGD
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(2d) RGD ligand - Cys right
(SEQ ID NO: 50)
RGDC
Note: This can be conjugated to CCA2 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(3a) Transferrin ligand
[anchoring domain (e.g., cationic anchoring domain) - linker
(GAPGAPGAP) - Targeting ligand]
(SEQ ID NO: 51)
RRRRRRRRR GAPGAPGAP THRPPMWSPVWP
(3b) Transferrin ligand
[Targeting ligand - linker (GAPGAPGAP) - anchoring domain
(e.g., cationic anchoring domain)]
(SEQ ID NO: 52)
THRPPMWSPVWP GAPGAPGAP RRRRRRRRR
(3c) Transferrin ligand - Cys left
(SEQ ID NO: 53)
CTHRPPMWSPVWP
(SEQ ID NO: 54)
CPTHRPPMWSPVWP
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(3d) Transferrin ligand - Cys right
(SEQ ID NO: 55)
THRPPMWSPVWPC
Note: This can be conjugated to CCA2 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(4a) E-selectin ligand [1-21]
[anchoring domain (e.g., cationic anchoring domain) - linker
(GAPGAPGAP) - Targeting ligand]
(SEQ ID NO: 56)
RRRRRRRRR GAPGAPGAP MIASQFLSALTLVLLIKESGA
(4b) E-selectin ligand [1-21]
[Targeting ligand - linker (GAPGAPGAP) - anchoring domain
(e.g., cationic anchoring domain)]
(SEQ ID NO: 57)
MIASQFLSALTLVLLIKESGA GAPGAPGAP RRRRRRRRR
(4c) E-selectin ligand [1-21]- Cys left
(SEQ ID NO: 58)
CMIASQFLSALTLVLLIKESGA
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(4d) E-selectin ligand [1-21]- Cys right
(SEQ ID NO: 59)
MIASQFLSALTLVLLIKESGAC
Note: This can be conjugated to CCA2 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(5a) FGF fragment [26-47]
[anchoring domain (e.g., cationic anchoring domain) - linker
(GAPGAPGAP) - Targeting ligand]
(SEQ ID NO: 60)
RRRRRRRRR GAPGAPGAP KNGGFFLRIHPDGRVDGVREKS
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(5b) FGF fragment [26-47]
[Targeting ligand - linker (GAPGAPGAP) - anchoring domain
(e.g., cationic anchoring domain)]
(SEQ ID NO: 61)
KNGGFFLRIHPDGRVDGVREKS GAPGAPGAP RRRRRRRRR
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(5c) FGF fragment [25-47]- Cys on left is native
(SEQ ID NO: 43)
CKNGGFFLRIHPDGRVDGVREKS
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(5d) FGF fragment [26-47]- Cys right
(SEQ ID NO: 44)
KNGGFFLRIHPDGRVDGVREKSC
Note: This can be conjugated to CCA2 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(SEQ ID NO: 2)
HGEGTFTSDLCKQMEEEAVRLFIEWLKNGGPSSGAPPPS
Note: This can be conjugated to CCA1 (see above) either via sulfhydryl
chemistry (e.g., a disulfide bond), amine-reactive chemistry or other
covalent conjugation chemistries including but not limited to
streptavadin-biotin, SpyTag/Catcher, gold-sulfur bonds, and the like.
(7a) Amino Acid Permease domain signature
[STAGC]-G-[PAG]-x(2,3)-[LIVMFYWA](2)-x-[LIVMFYW]-x-[LIVMFWSTAGC](2)-
[STAGC]-x(3)-[LIVMFYWT]-x-[LIVMST]-x(3)-[LIVMCTA]-[GA]-E-x(5)-[PSAL]\
(8a) C-Type Lectin domain signature
C-[LIVMFYATG]-x(5,12)-[WL]-{T}-[DNSR]-{C}-{LI}-C-x(5,6)-[FYWLIVSTA]-
[LIVMSTA]-C
(9a) Cadherin domain signature
[LIV]-x-[LIV]-x-D-x-N-D-[NH]-x-P
(10a) Caveolin domain signature
F-E-D-[LV]-I-A-[DE]-[PA]
(11a) Connexin domain signature
C-[DNH]-[TL]-x-[QT]-P-G-C-x(2)-[VAILl-C-[FY]-D
(12a) EGF-like domain signature
MRLLRRWAFAALLLSLLPTPGLGTQGPAGALRWGGLPQLGGPGAPEVTEPSRLVRESSGGE
VRKQQLDTRVRQEPPGGPPVHLAQVSFVIPAFNSNFTLDLELNHHLLSSQYVERHFSREGTTQHSTG
AGDHCYYQGKLRGNPHSFAALSTCQGLHGVFSDGNLTYIVEPQEVAGPWGAPQGPLPHLIYRTPLL
PDPLGCREPGCLFAVPAQSAPPNRPRLRRKRQVRRGHPTVHSETKYVELIVINDHQLFEQMRQSVVL
TSNFAKSVVNLADVIYKEQLNTRIVLVAMETWADGDKIQVQDDLLETLARLMVYRREGLPEPSDAT
HLFSGRTFQSTSSGAAYVGGICSLSHGGGVNEYGNMGAMAVTLAQTLGQNLGMMWNKHRSSAGD
CKCPDIWLGCIMEDTGFYLPRKFSRCSIDEYNQFLQEGGGSCLFNKPLKLLDPPECGNGFVEAGEEC
DCGSVQECSRAGGNCCKKCTLTHDAMCSDGLCCRRCKYEPRGVSCREAVNECDIAETCTGDSSQC
PPNLHKLDGYYCDHEQGRCYGGRCKTRDRQCQVLWGHAAADRFCYEKLNVEGTERGSCGRKGSG
WVQCSKQDVLCGFLLCVNISGAPRLGDLVGDISSVTFYHQGKELDCRGGHVQLADGSDLSYVEDG
TACGPNMLCLDHRCLPASAFNFSTCPGSGERRICSEIHGVCSNEGKCICQPDWTGKDCSIHNPLPTSPP
TGETERYKGPSGTNIIIGSIAGAVLVAAIVLGGTGWGFKNIRRGRSGGA
(13a) Endothelin family signature
C-x-C-x(4)-D-x(2)-C-x(2)-[FY]-C
(14a) G-protein coupled receptors family 1 signature
[GSTALIVMFYWC]-[GSTANCPDE]-{EDPKRH}-x-{PQ}-[LIVMNQGA]-{RK}-{RK}-
[LIVMFT]-[GSTANC]-[LIVMFYWSTAC]-[DENH]-R-[FYWCSH]-{PE}-x-[LIVM]
(15a) G-protein coupled receptors family 2 signature
family 2 signature 1: C-x(3)-[FYWLIV]-D-x(3,4)-C-[FW]-x(2)-[STAGV]-
x(8,9)-C-[PF]; family 2 signature 2: [QL]-G-[LMFCAV]-[LIVMFTA]-[LIV]-
x-[LIVFSTM]-[LIFHV]-[VFYHLG]-C-[LFYAVI]-x-[NKRQDS]-x(2)-[VAI]
(16a) G-protein coupled receptors family 3 signature
family 3 signature 1: [LV]-x-N-[LIVM](2)-x-L-F-x-I-[PA]-Q-[LIVM]-
[STA]-x-[STA](3)-[STAN];
family 3 signature 2: C-C-[FYW]-x-C-x(2)-C-x(4)-[FYW]-x(2,5)-[DNE]-
x(2)-[STAHENRI]-C-x(2)-C;
family 3 signature 3: [FLY]-N-[ED]-[STA]-K-x-[IV]-[STAG]-[FM]-[ST]-[MVL]
(17a) GPS domain profile
MAPPAARLALLSAAALTLAARPAPSPGLGPECFTANGADYRGTQNWTALQGGKPCLFWNE
TFQHPYNTLKYPNGEGGLGEHNYCRNPDGDVSPWCYVAEHEDGVYWKYCEIPACQMPGNLGCYK
DHGNPPPLTGTSKTSNKLTIQTCISFCRSQRFKFAGMESGYACFCGNNPDYWKYGEAASTECNSVCF
GDHTQPCGGDGRIILFDTLVGACGGNYSAMSSVVYSPDFPDTYATGRVCYWTIRVPGASHIHFSFPL
FDIRDSADMVELLDGYTHRVLARFHGRSRPPLSFNVSLDFVILYFFSDRINQAQGFAVLYQAVKEEL
PQERPAVNQTVAEVITEQANLSVSAARSSKVLYVITTSPSHPPQTVPGSNSWAPPMGAGSHRVEGW
TVYGLATLLILTVTAIVAKILLHVTFKSHRVPASGDLRDCHQPGTSGEIWSIFYKPSTSISIFKKKLKG
QSQQDDRNPLVSD
(18a) Glycophorin A signature
I-I-x-[GAC]-V-M-A-G-[LIVM](2)
(19a) HIG1 domain profile
MSTDTGVSLPSYEEDQGSKLIRKAKEAPFVPVGIAGFAAIVAYGLYKLKSRGNTKMSIHL/IH
MRVAAQGFVVGAMTVGMGYSMYREFWAKPKP
(20a) ITAM motif profile
MEHSTFLSGLVLATLLSQVSPFKIPIEELEDRVFVNCNTSITWVEGTVGTLLSDITRLDLGKRI
LDPRGIYRCNGTDIYKDKESTVQVHYRMCQSCVELDPATVAGIIVTDVIATLLLALGVFCFAGHETG
RLSGAADTQALLRNDQVYQPLRDRDDAQYSHLGGNWARNK
(21a) Immunoglobulins and major histocompatibility complex
proteins signature
[FY]-{L}-C-{PGAD}-[VA]-{LC}-H
(22a) Integrins alpha chain signature
[FYWS]-[RK]-x-G-F-F-x-R
(23a) Integrins beta chain cysteine rich domain signature
C-x-[GNQ]-x(1,3)-G-x-C-x-C-x(2)-C-x-C
(24a) Membrane attack complex/perforin domain signature
Y-x(6)-[FY]-G-T-H-[FY]
(25a) Receptor tyrosine kinase type II signature
[DN]-[LIV]-Y-x(3)-Y-Y-R
(26a) Receptor tyrosine kinase type III signature
G-x-H-x-N-[LIVM]-V-N-L-L-G-A-C-T
(27a) Receptor tyrosine kinase type V signature
C-x(2)-[DE]-G-[DEQKRG]-W-x(2,3)-[PAQ]-[LIVMT]-[GT]-x-C-x-C-x(2)-
G-[HFY]-[EQ]
(28a) SRCR domain signature
[GNRVM]-x(5)-[GLKA]-x(2)-[EQ]-x(6)-[WPS]-[GLKH]-x(2)-C-x(3)-
[FYW]-x(8)-[CM]-x(3)-G
(29a) Syndecans signature
[FY]-R-[IM]-[KR]-K(2)-D-E-G-S-Y
(30a) WD40 repeat signature
[LIVMSTAC]-[LIVMFYWSTAGC]-[LIMSTAG]-[LIVMSTAGC]-x(2)-[DN]-x-{P}-
[LIVMWSTAC]-{DP}-[LIVMFSTAG]-W-[DEN]-[LIVMPSTAGCN]

### (6a) Exendin (S11C) [1-39]

### Targeting Ligand

The targeting ligands in the present disclosure can be designed diagnostically-responsively following identification of the receptor profile of targeted cells. These targeting ligands may be peptides, peptoids, antibodies, aptamers, or other receptor-specific targeting molecules. In many embodiments, these targeting ligands are derived from native proteins or protein fragments where X-ray crystal structure data of a given protein (or protein homologue), or docking simulations of a given ligand to a measured or predicted protein structure, are used. In other embodiments, the targeting ligands are derived from antibodies, ScFvs, and the like. In other embodiments, the targeting ligands are derived from a SELEX or phage-display RNA/DNA aptamer or peptide libraries, respectively. In other embodiments, the targeting ligands are derived from other methods of combinatorial library prep of a random or natively-derived sequence/structure of polymer sequences [including peptides, peptoids, nucleotides, poly(B-amino esters), modified PEG sequences, LNAs, MNAs, PNAs and the like]. The “targeting ligands” are intended to represent a holistic set of targeting molecules designed for conferring cellular specificity for a combination of cellular receptor profiles, and can be combinatorially evaluated with a variety of nanoparticle or conjugation chemistries to create a cell/tissue/organ-specific delivery system for a given payload or set of payloads (e.g. CRISPR, TALEN, mRNA, small molecules).

Multiple targeting ligands patterned in specific densities along with optional stealth and/or linear/brushed glycoprotein motifs (as described elsewhere) may also be used to increase biodistributions and cell specificity, by limiting serum adsorption (protein corona formation, see, e.g., h followed by ttps://followed by ww followed by w.natu followed by re. co followed by m/articles/s41467-017-00600-w) to the ligand surface which otherwise limits cell-specific uptake. Regulation of particle clearance by macrophages may also be achieved through “eat me” and “don't eat me” cues on the particle surface, whereby CD47 and SIRPα normally interact and limit macrophage clearance of healthy cells. Fragments or mimetics (e.g. antibodies) of SIRPα may be presented upon the particle surface in order to limit macrophage clearance. Similar fragments or mimetics may be used as “receptor antagonistic” ligands that limit receptor-mediated endocytosis on targeted cells, while secondary sets of ligands (homo or heterovalent) may engage another cell's endocytotic machinery and cell specificity. Nanoparticles used in this way may also serve as intermediaries to cell-cell signaling, forming cell junctions (e.g. endothelial cell-immune junctions and the like) with biased uptake and gene-, gene edit-, and/or drug-mediated modification in the endocytosis-biased ligand-receptor pairing (e.g. the target cell population for genetic/other cellular reprogramming, such as with an immune cell engineered with an affinity marker). In other words, coupled with techniques for limiting non-specific serum adsorption, these embodiments can facilitate cell-specific targeting ligands (or combination of ligands) to confer 1) cell-specificity, 2) limited non-specific clearance of nanomaterials, and 3) active inhibition of macrophage/other cell uptake and protein corona formation in vivo, with an optional capacity for 4) cell-cell junction formation and biased reprogramming of a single target cell population. Broadly, the methods and uses for anchoring these targeting ligands to a universal set of gene editing, gene therapy and small molecule modalities represent clear innovation beyond the state of the art, in addition to significant innovations in “smart” composite nanomaterials and their architectures thereof, as well as the manufacturing, simulation, design and screening components thereof.

In some cases, a targeting ligand is conjugated (e.g., in some cases with a cleavable linker) directly to a payload—to deliver the payload. In some cases a targeting ligand is fused to a charged domain (detailed elsewhere herein), e.g., where the charged domain interacts with a payload. In some cases, a targeting ligand is associated with (e.g., through electrostatic interactions, via direct conjugation, via lipids, and the like) a delivery vehicle such as a solid particle core nanoparticle or a nanoparticle having a core that comprises polymers (e.g., a nanoparticle having cationic/anionic polymers, a cationic polypeptide, and the like)—for example, for the targeted delivery of a payload. In some cases a targeting ligand can serve it's own purpose without delivering a payload—as an example, an IL2 fragment (or IL-2-PEG) can be used.

A variety of targeting ligands (e.g., as part of a subject delivery molecule, e.g., as part of a nanoparticle) can be used (e.g., at any desired surface density when used as part of a nanoparticle) and numerous different targeting ligands are envisioned. In some embodiments the targeting ligand is a fragment (e.g., a binding domain) of a wild type protein. For example, in some cases a peptide targeting ligand of a subject delivery molecule can have a length of from 4-50 amino acids (e.g., from 4-40, 4-35, 4-30, 4-25, 4-20, 4-15, 5-50, 5-40, 5-35, 5-30, 5-25, 5-20, 5-15, 7-50, 7-40, 7-35, 7-30, 7-25, 7-20, 7-15, 8-50, 8-40, 8-35, 8-30, 8-25, 8-20, or 8-15 amino acids). The targeting ligand can be a fragment of a wild type protein, but in some cases has a mutation (e.g., insertion, deletion, substitution) relative to the wild type amino acid sequence (i.e., a mutation relative to a corresponding wild type protein sequence). For example, a targeting ligand can include a mutation that increases or decreases binding affinity with a target cell surface protein. Once 5-200 amino acids (e.g., from 5-150, 5-100, 5-80, 15-200, 15-150, 15-100, 15-80, 30-200, 30-150, 30-100, 30-80, 50-200, 50-150, 50-100, or 50-80 amino acids) within a binding pocket of a given receptor are identified, libraries of peptide targeting ligands of from 4-50 amino acids (e.g., from 4-40, 4-35, 4-30, 4-25, 4-20, 4-15, 5-50, 5-40, 5-35, 5-30, 5-25, 5-20, 5-15, 7-50, 7-40, 7-35, 7-30, 7-25, 7-20, 7-15, 8-50, 8-40, 8-35, 8-30, 8-25, 8-20, or 8-15 amino acids) can be generated (e.g. 1, 2, 3, 4, 5, 10, 15, 30, 50 or 100 targeting ligands per receptor) with variable anchor and linker motifs and nanoparticle-binding chemistries. These libraries of peptide targeting ligands may be screened according to a variety of nanoparticle formulations as disclosed herein (e.g. variable D:L isomer ratios, molecular weights, charges and compositions of cationic/anionic polymers; lipid embodiments and alternative nanoparticle chemistries may also be used), either decorating a pre-formed particle or directly forming the particle through directed self-assembling interactions (e.g. electrostatic, DNA origami templates, etc.). The best performing particles, as determined by their physicochemical and biological properties (e.g. size, charge, payload stability, cellular internalization, cellular specificity, cellular gene expression/editing), can be selected and in some cases further iterated around for increased cell/tissue/organ-specific behavior.

In some cases the targeting ligand is an antigen-binding region of an antibody (F(ab)). In some cases the targeting ligand is an ScFv. “Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. For a review of scFv see Pluckthun, in The Pharmacology of Monoclonal Antibodies, vol. 113, Rosenburg and Moore eds., Springer-Verlag, New York, pp. 269-315 (1994).

In some cases a targeting ligand includes a viral glycoprotein, which in some cases binds to ubiquitous surface markers such as heparin sulfate proteoglycans, and may induce micropinocytosis (and/or macropinocytosis) in some cell populations through membrane ruffling associated processes. Poly(L-arginine) is another example targeting ligand that can also be used for binding to surface markers such as heparin sulfate proteoglycans.

In some cases a targeting ligand is coated upon a particle surface (e.g., nanoparticle surface) either electrostatically or utilizing covalent modifications to the particle surface or one or more polymers on the particle surface. In some cases, a targeting ligand can include a mutation that adds a cysteine residue, which can facilitate conjugation to a linker and/or an anchoring domain (e.g., cationic anchoring domain). For example, cysteine can be used for crosslinking (conjugation) via sulfhydryl chemistry (e.g., a disulfide bond) and/or amine-reactive chemistry.

In some cases, a targeting ligand includes an internal cysteine residue. In some cases, a targeting ligand includes a cysteine residue at the N- and/or C-terminus. In some cases, in order to include a cysteine residue, a targeting ligand is mutated (e.g., insertion or substitution), e.g., relative to a corresponding wild type sequence. As such, any of the targeting ligands described herein can be modified by inserting and/or substituting in a cysteine residue (e.g., internal, N-terminal, C-terminal insertion of or substitution with a cysteine residue).

By “corresponding” wild type sequence is meant a wild type sequence from which the subject sequence was or could have been derived (e.g., a wild type protein sequence having high sequence identity to the sequence of interest). In some cases, a “corresponding” wild type sequence is one that has 85% or more sequence identity (e.g., 90% or more, 92% or more, 95% or more, 97% or more, 98% or more, 99% or more, 99.5% or more, or 100% sequence identity) over the amino acid stretch of interest. For example, for a targeting ligand that has one or more mutations (e.g., substitution, insertion) but is otherwise highly similar to a wild type sequence, the amino acid sequence to which it is most similar may be considered to be a corresponding wild type amino acid sequence.

A corresponding wild type protein/sequence does not have to be 100% identical (e.g., can be 85% or more identical, 90% or more identical, 95% or more identical, 98% or more identical, 99% or more identical, etc.) (outside of the position(s) that is modified), but the targeting ligand and corresponding wild type protein (e.g., fragment of a wild protein) can bind to the intended cell surface protein, and retain enough sequence identity (outside of the region that is modified) that they can be considered homologous. The amino acid sequence of a “corresponding” wild type protein sequence can be identified/evaluated using any convenient method (e.g., using any convenient sequence comparison/alignment software such as BLAST, MUSCLE, T-COFFEE, etc.).

Examples of targeting ligands that can be used as part of a surface coat (e.g., as part of a delivery molecule of a surface coat) include, but are not limited to, those listed in Table 1. Examples of targeting ligands that can be used as part of a subject delivery molecule include, but are not limited to, those listed in Table 3 (many of the sequences listed in Table 3 include the targeting ligand (e.g., SNRWLDVK for row 2) conjugated to a cationic polypeptide domain, e.g., 9R, 6R, etc., via a linker (e.g., GGGGSGGGGS). Examples of amino acid sequences that can be included in a targeting ligand include, but are not limited to: NPKLTRMLTFKFY (SEQ ID NO: xx) (IL2), TSVGKYPNTGYYGD (SEQ ID NO: xx) (CD3), SNRWLDVK (Siglec), EKFILKVRPAFKAV (SEQ ID NO: xx) (SCF); EKFILKVRPAFKAV (SEQ ID NO: xx) (SCF), EKFILKVRPAFKAV (SEQ ID NO: xx) (SCF), SNYSIIDKLVNIVDDLVECVKENS (SEQ ID NO: xx) (cKit), and Ac-SNYSAibADKAibANAibADDAibAEAibAKENS (SEQ ID NO: xx) (cKit). Thus in some cases a targeting ligand includes an amino acid sequence that has 85% or more (e.g., 90% or more, 95% or more, 98% or more, 99% or more, or 100%) sequence identity with NPKLTRMLTFKFY (SEQ ID NO: xx) (IL2), TSVGKYPNTGYYGD (SEQ ID NO: xx) (CD3), SNRWLDVK (Siglec), EKFILKVRPAFKAV (SEQ ID NO: xx) (SCF); EKFILKVRPAFKAV (SEQ ID NO: xx) (SCF), EKFILKVRPAFKAV (SEQ ID NO: xx) (SCF), or SNYSIIDKLVNIVDDLVECVKENS (SEQ ID NO: xx) (cKit).

TABLE 1
depicts non-limiting classes of targeting ligand and conserved receptor domains.
The proteins represent either the targeting ligand, or the receptor in question.
For receptor families, this data is useful for generating predictions of
complementary ligands where crystal structure or other structural modeling data,
such as through homologous sequence modeling, is available. These ligands may be
modeled through numerous approaches, including de novo modeling based on protein
family homologues of overexpressed receptors on a target cell/tissue/organ. Synthesis
of existing protein domains and other forms of targeted library generation (e.g.
antibodies, SELEX, and the like) may also be used. These ligands may be used as small
molecule drug conjugates, nanoparticle surface modifications, and for a variety of
purposes in drug and gene delivery requiring targeting of specific cells or specific
combinations of cells/tissues/organs. The ligands may be synthesized either
recombinantly or through flow-based high-throughput peptide synthesis.

| Conserved | | | SEQ |
|---|---|---|---|
| Receptor | Targeting | | ID |
| Domain | Ligand | Sequence | NO: |
| Family B | Exendin | HGEGTFTSDLSKQMEEEAVRLFIEWLKNG | 1 |
| GPCR | | GPSSGAPPPS | |
| | Exendin (S11C) | HGEGTFTSDLCKQMEEEAVRLFIEWLKNG | 2 |
| | | GPSSGAPPPS | |
| FGF receptor | FGF fragment | KRLYCKNGGFFLRIHPDGRVDGVREKSDP | 3 |
| | | HIKLQLQAEERGVVSIKGVCANRYLAMKE | |
| | | DGRLLASKCVTDECFFFERLESNNYNTY | |
| | FGF fragment | KNGGFFLRIHPDGRVDGVREKS | 4 |
| | FGF fragment | HFKDPK | 5 |
| | FGF fragment | LESNNYNT | 6 |
| | E-selectin | MIASQFLSALTLVLLIKESGA | 7 |
| | L-selectin | MVFPWRCEGTYWGSRNILKLWVWTLLCC | 8 |
| | | DFLIHHGTHC | |
| | | MIFPWKCQSTQRDLWNIFKLWGWTMLCC | 9 |
| | | DFLAHHGTDC | |
| | | MIFPWKCQSTQRDLWNIFKLWGWTMLCC | 10 |
| P-selectin | PSGL-1 | MAVGASGLEGDKMAGAMPLQLLLLLILL | 271 |
| | (SELPLG) | GPGNSLQLWDTWADEAEKALGPLLARDR | |
| | | RQATEYEYLDYDFLPETEPPEMLRNSTDT | |
| | | TPLTGPGTPESTTVEPAARRSTGLDAGGA | |
| | | VTELTTELANMGNLSTDSAAMEIQTTQPA | |
| | | ATEAQTTQPVPTEAQTTPLAATEAQTTRL | |
| | | TATEAQTTPLAATEAQTTPPAATEAQTTQ | |
| | | PTGLEAQTTAPAAMEAQTTAPAAMEAQT | |
| | | TPPAAMEAQTTQTTAMEAQTTAPEATEA | |
| | | QTTQPTATEAQTTPLAAMEALSTEPSATE | |
| | | ALSMEPTTKRGLFIPFSVSSVTHKGIPMAA | |
| | | SNLSVNYPVGAPDHISVKQCLLAILILALV | |
| | | ATIFFVCTVVLAVRLSRKGHMYPVRNYSP | |
| | | TEMVCISSLLPDGGEGPSATANGGLSKAK | |
| | | SPGLTPEPREDREGDDLTLHSFLP | |
| E-selectin | ESL-1 | MAACGRVRRMERLSAALHLLLLFAAGAE | 272 |
| | (GLG1) | KLPGQGVHSQGQGPGANFVSFVGQAGGG | |
| | | GPAGQQLPQLPQSSQLQQQQQQQQQQQQ | |
| | | PQPPQPPFPAGGPPARRGGAGAGGGWKL | |
| | | AEEESCREDVTRVCPKHTWSNNLAVLECL | |
| | | QDVREPENEISSDCNHLLWNYKLNLTTDP | |
| | | KFESVAREVCKSTITEIKECADEPVGKGY | |
| | | MVSCLVDHRGNITEYQCHQYITKMTAIIFS | |
| | | DYRLICGFMDDCKNDINILKCGSIRLGEKD | |
| | | AHSQGEVVSCLEKGLVKEAEEREPKIQVS | |
| | | ELCKKAILRVAELSSDDFHLDRHLYFACR | |
| | | DDRERFCENTQAGEGRVYKCLFNHKFEES | |
| | | MSEKCREALTTRQKLIAQDYKVSYSLAKS | |
| | | CKSDLKKYRCNVENLPRSREARLSYLLMC | |
| | | LESAVHRGRQVSSECQGEMLDYRRMLME | |
| | | DFSLSPEIILSCRGEIEHHCSGLHRKGRTLH | |
| | | CLMKVVRGEKGNLGMNCQQALQTLIQET | |
| | | DPGADYRIDRALNEACESVIQTACKHIRSG | |
| | | DPMILSCLMEHLYTEKMVEDCEHRLLELQ | |
| | | YFISRDWKLDPVLYRKCQGDASRLCHTH | |
| | | GWNETSEFMPQGAVFSCLYRHAYRTEEQ | |
| | | GRRLSRECRAEVQRILHQRAMDVKLDPAL | |
| | | QDKCLIDLGKWCSEKTETGQELECLQDHL | |
| | | DDLVVECRDIVGNLTELESEDIQIEALLMR | |
| | | ACEPIIQNFCHDVADNQIDSGDLMECLIQN | |
| | | KHQKDMNEKCAIGVTHFQLVQMKDFRFS | |
| | | YKFKMACKEDVLKLCPNIKKKVDVVICLS | |
| | | TTVRNDTLQEAKEHRVSLKCRRQLRVEEL | |
| | | EMTEDIRLEPDLYEACKSDIKNFCSAVQY | |
| | | GNAQIIECLKENKKQLSTRCHQKVFKLQE | |
| | | TEMMDPELDYTLMIRVCKQMIKRFCPEAD | |
| | | SKTMLQCLKQNKNSELMDPKCKQMITKR | |
| | | QITQNTDYRLNPMLRKACKADIPKFCHGI | |
| | | LTKAKDDSELEGQVISCLKLRYADQRLSS | |
| | | DCEDQIRIIIQESALDYRLDPQLQLHCSDEI | |
| | | SSLCAEEAAAQEQTGQVEECLKVNLLKIK | |
| | | TELCKKEVLNMLKESKADIFVDPVLHTAC | |
| | | ALDIKHHCAAITPGRGRQMSCLMEALEDK | |
| | | RVRLQPECKKRLNDRIEMWSYAAKVAPA | |
| | | DGFSDLAMQVMTSPSKNYILSVISGSICILF | |
| | | LIGLMCGRITKRVTRELKDRLQYRSETMA | |
| | | YKGLVWSQDVTGSPA | |
| | PSGL-1 | See above | 271 |
| | (SELPLG) | | |
| | CD44 | MDKFWWHAAWGLCLVPLSLAQIDLNITC | 273 |
| | | RFAGVFHVEKNGRYSISRTEAADLCKAFN | |
| | | STLPTMAQMEKALSIGFETCRYGFIEGHV | |
| | | VIPRIHPNSICAANNTGVYILTSNTSQYDTY | |
| | | CFNASAPPEEDCTSVTDLPNAFDGPITITIV | |
| | | NRDGTRYVQKGEYRTNPEDIYPSNPTDDD | |
| | | VSSGSSSERSSTSGGYIFYTFSTVHPIPDED | |
| | | SPWITDSTDRIPATTLMSTSATATETATKR | |
| | | QETWDWFSWLFLPSESKNHLHTTTQMAG | |
| | | TSSNTISAGWEPNEENEDERDRHLSFSGSG | |
| | | IDDDEDFISSTISTTPRAFDHTKQNQDWTQ | |
| | | WNPSHSNPEVLLQTTTRMTDVDRNGTTA | |
| | | YEGNWNPEAHPPLIHHEHHEEEETPHSTST | |
| | | IQATPSSTTEETATQKEQWFGNRWHEGYR | |
| | | QTPKEDSHSTTGTAAASAHTSHPMQGRTT | |
| | | PSPEDSSWTDFFNPISHPMGRGHQAGRRM | |
| | | DMDSSHSITLQPTANPNTGLVEDLDRTGP | |
| | | LSMTTQQSNSQSFSTSHEGLEEDKDHPTTS | |
| | | TLTSSNRNDVTGGRRDPNHSEGSTTLLEG | |
| | | YTSHYPHTKESRTFIPVTSAKTGSFGVTAV | |
| | | TVGDSNSNVNRSLSGDQDTFHPSGGSHTT | |
| | | HGSESDGHSHGSQEGGANTTSGPIRTPQIP | |
| | | EWLIILASLLALALILAVCIAVNSRRRCGQ | |
| | | KKKLVINSGNGAVEDRKPSGLNGEASKSQ | |
| | | EMVHLVNKESSETPDQFMTADETRNLQN | |
| | | VDMKIGV | |
| | DR3 | MEQRPRGCAAVAAALLLVLLGARAQGGT | 274 |
| | (TNFRSF25) | RSPRCDCAGDFHKKIGLFCCRGCPAGHYL | |
| | | KAPCTEPCGNSTCLVCPQDTFLAWENHHN | |
| | | SECARCQACDEQASQVALENCSAVADTR | |
| | | CGCKPGWFVECQVSQCVSSSPFYCQPCLD | |
| | | CGALHRHTRLLCSRRDTDCGTCLPGFYEH | |
| | | GDGCVSCPTPPPSLAGAPWGAVQSAVPLS | |
| | | VAGGRVGVFWVQVLLAGLVVPLLLGATL | |
| | | TYTYRHCWPHKPLVTADEAGMEALTPPP | |
| | | ATHLSPLDSAHTLLAPPDSSEKICTVQLVG | |
| | | NSWTPGYPETQEALCPQVTWSWDQLPSR | |
| | | ALGPAAAPTLSPESPAGSPAMMLQPGPQL | |
| | | YDVMDAVPARRWKEFVRTLGLREAEIEA | |
| | | VEVEIGRFRDQQYEMLKRWRQQQPAGLG | |
| | | AVYAALERMGLDGCVEDLRSRLQRGP | |
| | LAMP1 | MAAPGSARRPLLLLLLLLLLGLMHCASAA | 275 |
| | | MFMVKNGNGTACIMANFSAAFSVNYDTK | |
| | | SGPKNMTFDLPSDATVVLNRSSCGKENTS | |
| | | DPSLVIAFGRGHTLTLNFTRNATRYSVQL | |
| | | MSFVYNLSDTHLFPNASSKEIKTVESITDIR | |
| | | ADIDKKYRCVSGTQVHMNNVTVTLHDAT | |
| | | IQAYLSNSSFSRGETRCEQDRPSPTTAPPAP | |
| | | PSPSPSPVPKSPSVDKYNVSGTNGTCLLAS | |
| | | MGLQLNLTYERKDNTTVTRLLNINPNKTS | |
| | | ASGSCGAHLVTLELHSEGTTVLLFQFGMN | |
| | | ASSSRFFLQGIQLNTILPDARDPAFKAANG | |
| | | SLRALQATVGNSYKCNAEEHVRVTKAFS | |
| | | VNIFKVWVQAFKVEGGQFGSVEECLLDE | |
| | | NSMLIPIAVGGALAGLVLIVLIAYLVGRKR | |
| | | SHAGYQTI | |
| | LAMP2 | MVCFRLFPVPGSGLVLVCLVLGAVRSYAL | 276 |
| | | ELNLTDSENATCLYAKWQMNFTVRYETT | |
| | | NKTYKTVTISDHGTVTYNGSICGDDQNGP | |
| | | KIAVQFGPGFSWIANFTKAASTYSIDSVSF | |
| | | SYNTGDNTTFPDAEDKGILTVDELLAIRIP | |
| | | LNDLFRCNSLSTLEKNDVVQHYWDVLVQ | |
| | | AFVQNGTVSTNEFLCDKDKTSTVAPTIHT | |
| | | TVPSPTTTPTPKEKPEAGTYSVNNGNDTCL | |
| | | LATMGLQLNITQDKVASVININPNTTHSTG | |
| | | SCRSHTALLRLNSSTIKYLDFVFAVKNENR | |
| | | FYLKEVNISMYLVNGSVFSIANNNLSYWD | |
| | | APLGSSYMCNKEQTVSVSGAFQINTFDLR | |
| | | VQPFNVTQGKYSTAQDCSADDDNFLVPIA | |
| | | VGAALAGVLILVLLAYFIGLKHHHAGYEQF | |
| | Mac2-BP | MTPPRLFWVWLLVAGTQGVNDGDMRLA | 277 |
| | (galectin 3 | DGGATNQGRVEIFYRGQWGTVCDNLWD | |
| | binding protein) | LTDASVVCRALGFENATQALGRAAFGQG | |
| | (LGALS3BP) | SGPIMLDEVQCTGTEASLADCKSLGWLKS | |
| | | NCRHERDAGVVCTNETRSTHTLDLSRELS | |
| | | EALGQIFDSQRGCDLSISVNVQGEDALGFC | |
| | | GHTVILTANLEAQALWKEPGSNVTMSVD | |
| | | AECVPMVRDLLRYFYSRRIDITLSSVKCFH | |
| | | KLASAYGARQLQGYCASLFAILLPQDPSF | |
| | | QMPLDLYAYAVATGDALLEKLCLQFLAW | |
| | | NFEALTQAEAWPSVPTDLLQLLLPRSDLA | |
| | | VPSELALLKAVDTWSWGERASHEEVEGL | |
| | | VEKIRFPMMLPEELFELQFNLSLYWSHEA | |
| | | LFQKKTLQALEFHTVPFQLLARYKGLNLT | |
| | | EDTYKPRIYTSPTWSAFVTDSSWSARKSQ | |
| | | LVYQSRRGPLVKYSSDYFQAPSDYRYYPY | |
| | | QSFQTPQHPSFLFQDKRVSWSLVYLPTIQS | |
| | | CWNYGFSCSSDELPVLGLTKSGGSDRTIA | |
| | | YENKALMLCEGLFVADVTDFEGWKAAIP | |
| | | SALDTNSSKSTSSFPCPAGHFNGFRTVIRPF | |
| | | YLTNSSGVD | |
| Transferrin | Transferrin | THRPPMWSPVWP | 11 |
| receptor | ligand | | |
| α5β1 integrin | α5β1 ligand | RRETAWA | 12 |
| | | RGD | |
| | | RGDGW | 181 |
| integrin | Integrin binding | (Ac)-GCGYGRGDSPG-(NH2) | 188 |
| | peptide | GCGYGRGDSPG | 182 |
| α5β3 integrin | α5β3 ligand | DGARYCRGDCFDG | 187 |
| | rabies virus | YTIWMPENPRPGTPCDIFTNSRGKRASNG | 183 |
| | glycoprotein | GGG | |
| | (RVG) | | |
| c-Kit receptor | stem cell factor | EGICRNRVTNNVKDVTKLVANLPKDYMIT | 184 |
| (CD117) | (SCF) | LKYVPGMDVLPSHCWISEMVVQLSDSLT | |
| | | DLLDKFSNISEGLSNYSIIDKLVNIVDDLVE | |
| | | CVKENSSKDLKKSFKSPEPRLFTPEEFFRIF | |
| | | NRSIDAFKDFVVASETSDCVVSSTLSPEKD | |
| | | SRVSVTKPFMLPPVA | |
| CD27 | CD70 | PEEGSGCSVRRRPYGCVLRAALVPLVAGL | 185 |
| | | VICLVVCIQRFAQAQQQLPLESLGWDVAE | |
| | | LQLNHTGPQQDPRLYWQGGPALGRSFLH | |
| | | GPELDKGQLRIHRDGIYMVHIQVTLAICSS | |
| | | TTASRHHPTTLAVGICSPASRSISLLRLSFH | |
| | | QGCTIASQRLTPLARGDTLCTNLTGTLLPS | |
| | | RNTDETFFGVQWVRP | |
| CD150 | SH2 domain- | SSGLVPRGSHMDAVAVYHGKISRETGEKL | 186 |
| | containing | LLATGLDGSYLLRDSESVPGVYCLCVLYH | |
| | protein 1A | GYIYTYRVSQTETGSWSAETAPGVHKRYF | |
| | (SH2D1A) | RKIKNLISAFQKPDQGIVIPLQYPVEKKSSA | |
| | | RSTQGTTGIREDPDVCLKAP | |
| IL2R | IL2 | NPKLTRMLTFKFY | |
| CD3 | Cde3-epsilon | NFYLYRA-NH2 | |
| CD8 | peptide-HLA- | RYPLTFGWCF-NH2 | |
| | A*2402 | | |
| CD8 | | FTDNAKTI | |
| CD28 | CD80 | VVLKYEKDAFKR | |
| CD28 | CD86 | ENLVLNE | |
| Angiopoietin- | ANGPTL5- | MMSPSQASLLFLNVCIFICGEAVQG | |
| Like Protein | derived signal | | |
| Receptors | peptide sequence | | |
| Amino acid | | [STAGC]-G-[PAG]-x(2,3)-[LIVMFYWA](2)- | |
| permease | | x-[LIVMFYW]-x-[LIVMFWSTAGC](2)- | |
| domain | | [STAGC]-x(3)-[LIVMFYWT]-x-[LIVMST]- | |
| signature | | x(3)-[LIVMCTA]-[GA]-E-x(5)-[PSAL] | |
| C-type lectin | | C-[LIVMFYATG]-x(5,12)-[WL]-{T}-[DNSR]- | |
| domain | | {C}-{LI}-C-x(5,6)-[FYWLIVSTA]- | |
| signature | | [LIVMSTA]-C | |
| Cadherin | | [LIV]-x-[LIV]-x-D-x-N-D-[NH]-x-P | |
| domain | | | |
| signature | | | |
| Caveolin | | F-E-D-[LV]-I-A-[DE]-[PA] | |
| domain | | | |
| signature | | | |
| Connexin | | C-[DNH]-[TL]-x-[QT]-P-G-C-x(2)-[VAIL]-C- | |
| domain | | [FY]-D | |
| signature | | | |
| EGF-like | | MRLLRRWAFAALLLSLLPTPGLGTQGPAG | |
| domain | | ALRWGGLPQLGGPGAPEVTEPSRLVRESS | |
| signature | | GGEVRKQQLDTRVRQEPPGGPPVHLAQV | |
| | | SFVIPAFNSNFTLDLELNHHLLSSQYVERH | |
| | | FSREGTTQHSTGAGDHCYYQGKLRGNPH | |
| | | SFAALSTCQGLHGVFSDGNLTYIVEPQEV | |
| | | AGPWGAPQGPLPHLIYRTPLLPDPLGCREP | |
| | | GCLFAVPAQSAPPNRPRLRRKRQVRRGHP | |
| | | TVHSETKYVELIVINDHQLFEQMRQSVVL | |
| | | TSNFAKSVVNLADVIYKEQLNTRIVLVAM | |
| | | ETWADGDKIQVQDDLLETLARLMVYRRE | |
| | | GLPEPSDATHLFSGRTFQSTSSGAAYVGGI | |
| | | CSLSHGGGVNEYGNIVIGAMAVTLAQTLG | |
| | | QNLGMMWNKHRSSAGDCKCPDIWLGCI | |
| | | MEDTGFYLPRKFSRCSIDEYNQFLQEGGG | |
| | | SCLFNKPLKLLDPPECGNGFVEAGEECDC | |
| | | GSVQECSRAGGNCCKKCTLTHDAMCSDG | |
| | | LCCRRCKYEPRGVSCREAVNECDIAETCT | |
| | | GDSSQCPPNLHKLDGYYCDHEQGRCYGG | |
| | | RCKTRDRQCQVLWGHAAADRFCYEKLN | |
| | | VEGTERGSCGRKGSGWVQCSKQDVLCGF | |
| | | LLCVNISGAPRLGDLVGDISSVTFYHQGKE | |
| | | LDCRGGHVQLADGSDLSYVEDGTACGPN | |
| | | MLCLDHRCLPASAFNFSTCPGSGERRICSH | |
| | | HGVCSNEGKCICQPDWTGKDCSIHNPLPT | |
| | | SPPTGETERYKGPSGTNIIIGSIAGAVLVAA | |
| | | IVLGGTGWGFKNIRRGRSGGA | |
| Endothelin | | C-x-C-x(4)-D-x(2)-C-x(2)-[FY]-C | |
| family | | | |
| signature | | | |
| G-protein | | [GSTALIVMFYWC]-[GSTANCPDE]- | |
| coupled | | {EDPKRH}-x-{PQ}-[LIVMNQGA]-{RK}- | |
| receptors | | {RK}-[LIVMFT]-[GSTANC]- | |
| family 1 | | [LIVMFYWSTAC]-[DENH]-R-[FYWCSH]- | |
| signature | | {PE}-x-[LIVM] | |
| G-protein | | family 2 signature 1: C-x(3)-[FYWLIV]-D- | |
| coupled | | x(3,4)-C-[FW]-x(2)-[STAGV]-x(8,9)-C-[PF]; | |
| receptors | | family 2 signature 2: [QL]-G-[LMFCAV]- | |
| family 2 | | [LIVMFTA]-[LIV]-x-[LIVFSTM]-[LIFHV]- | |
| signature | | [VFYHLG]-C-[LFYAVI]-x-[NKRQDS]-x(2)- | |
| | | [VAI] | |
| G-protein | | family 3 signature 1: [LV]-x-N-[LIVM](2)-x-L- | |
| coupled | | F-x-I-[PA]-Q-[LIVM]-[STA]-x-[STA](3)- | |
| receptors | | [STAN]; family 3 signature 2: C-C-[FYW]-x- | |
| family 3 | | C-x(2)-C-x(4)-[FYW]-x(2,5)-[DNE]-x(2)- | |
| signature | | [STAHENRI]-C-x(2)-C; | |
| | | family 3 signature 3: [FLY]-N-[ED]-[STA]-K- | |
| | | x-[IV]-[STAG]-[FM]-[ST]-[MVL] | |
| GPS domain | | MAPPAARLALLSAAALTLAARPAPSPGLG | |
| profile | | PECFTANGADYRGTQNWTALQGGKPCLF | |
| | | WNETFQHPYNTLKYPNGEGGLGEHNYCR | |
| | | NPDGDVSPWCYVAEHEDGVYWKYCEIPA | |
| | | CQMPGNLGCYKDHGNPPPLTGTSKTSNKL | |
| | | TIQTCISFCRSQRFKFAGMESGYACFCGNN | |
| | | PDYWKYGEAASTECNSVCFGDHTQPCGG | |
| | | DGRIILFDTLVGACGGNYSAMSSVVYSPD | |
| | | FPDTYATGRVCYWTIRVPGASHIHFSFPLF | |
| | | DIRDSADMVELLDGYTHRVLARFHGRSRP | |
| | | PLSFNVSLDFVILYFFSDRINQAQGFAVLY | |
| | | QAVKEELPQERPAVNQTVAEVITEQANLS | |
| | | VSAARSSKVLYVITTSPSHPPQTVPGSNSW | |
| | | APPMGAGSHRVEGWTVYGLATLLILTVT | |
| | | AIVAKILLHVTFKSHRVPASGDLRDCHQP | |
| | | GTSGEIWSIFYKPSTSISIFKKKLKGQSQQD | |
| | | DRNPLVSD | |
| Glycophorin A | | I-I-x-[GAC]-V-M-A-G-[LIVM](2) | |
| signature | | | |
| HIG1 domain | | MSTDTGVSLPSYEEDQGSKLIRKAKEAPF | |
| profile | | VPVGIAGFAAIVAYGLYKLKSRGNTKMSI | |
| | | HL\IHMRVAAQGFVVGAMTVGMGYSMY | |
| | | REFWAKPKP | |
| ITAM motif | | MEHSTFLSGLVLATLLSQVSPFKIPIEELED | |
| profile | | RVFVNCNTSITWVEGTVGTLLSDITRLDLG | |
| | | KRILDPRGIYRCNGTDIYKDKESTVQVHY | |
| | | RMCQSCVELDPATVAGIIVTDVIATLLLAL | |
| | | GVFCFAGHETGRLSGAADTQALLRNDQV | |
| | | YQPLRDRDDAQYSHLGGNWARNK | |
| Immunoglobulins | | [FY]-{L }-C-{PGAD}-[VA]-{LC}-H | |
| and major | | | |
| histocompati- | | | |
| bility complex | | | |
| proteins | | | |
| signature | | | |
| Integrins alpha | | [FYWS]-[RK]-x-G-F-F-x-R | |
| chain signature | | | |
| Integrins beta | | C-x-[GNQ]-x(1,3)-G-x-C-x-C-x(2)-C-x-C | |
| chain cysteine | | | |
| rich domain | | | |
| signature | | | |
| Membrane | | Y-x(6)-[FY]-G-T-H-[FY] | |
| attack | | | |
| complex/perfor | | | |
| in domain | | | |
| signature | | | |
| Receptor | | [DN]-[LIV]-Y-x(3)-Y-Y-R | |
| tyrosine kinase | | | |
| type II | | | |
| signature | | | |
| Receptor | | G-x-H-x-N-[LIVM]-V-N-L-L-G-A-C-T | |
| tyrosine kinase | | | |
| type III | | | |
| signature | | | |
| Receptor | | C-x(2)-[DE]-G-[DEQKRG]-W-x(2,3)-[PAQ]- | |
| tyrosine kinase | | [LIVMT]-[GT]-x-C-x-C-x(2)-G-[HFY]-[EQ] | |
| type V | | | |
| signature | | | |
| SRCR domain | | [GNRVM]-x(5)-[GLKA]-x(2)-[EQ]-x(6)- | |
| signature | | [WPS]-[GLKH]-x(2)-C-x(3)-[FYW]-x(8)- | |
| | | [CM]-x(3)-G | |
| Syndecans | | [FY]-R-[IM]-[KR]-K(2)-D-E-G-S-Y | |
| signature | | | |
| WD40 repeats | | [LIVMSTAC]-[LIVMFYWSTAGC]- | |
| signature | | [LIMSTAG]-[LIVMSTAGC]-x(2)-[DN]-x- | |
| | | {P }-[LIVMWSTAC]-{DP}-[LIVMFSTAG]- | |
| | | W-[DEN]-[LIVMFSTAGCN] | |

One non-limiting example of a multifunctional peptide sequence (variable anchor, linker and ligand domains with cell-specific matrix metalloprotease degradation behavior) is as follows:

Endo_X_Alexa594_4GS_3KRK_2_N_1 (cl24):
KKKRKKKKRKGGGGSCGGGGSSFKFLFDIIKKIAES-[optional
ligand]

FIG. 18A depicts this peptide.
This peptide serves many purposes:
KKKRKKKKRK—Anchor domain. Electrostatic-phase domain for genetic/protein payload condensation with importin-binding sequence for nuclear targeting. The N-terminus can also be utilized as a covalent modification to a small molecule drug, protein, or binding surface (as detailed elsewhere). Alternative sequences may be net-cationic, net-anionic, histone tail peptides, alternative NLS or subcellular trafficking/release sequences, and additional embodiments for reversible-charged and reversibly-binding electrostatic domains. This domain may also be replaced with a variety of covalent coupling techniques to alternative entities as described elsewhere.
GGGGSCGGGGSS—Flexible linker/spacer domain between electrostatic-phase domain and subsequent functional domain. This particular sequence includes a cysteine residue for linking to maleimide moieties. It may also be used to form cross-chain crosslinks between individual anchor-linker-ligand pairings. In this case, in contrast to H2A-3C and other cysteine-substituted histone tail peptides/cationic motifs utilized in our “core condensation” studies with cationic and anionic polypeptides, AlexaFluor594 occupies 100% of Cys residues on the linker domains. In alternative embodiments, the release of cross-chain crosslinks from a nanoparticle is believed to namely be mediated through glutathione activity and the stability of these complexes is shown elsewhere where mRNA condensation data (SYBR inclusion/exclusion curves) are used to show extended serum stability of nanoparticle complexes utilizing interspersed cysteine substitutions (e.g. cysteine-substituted histone tail peptides, cysteine-substituted anchor domains, cysteine-substituted linker domains, cysteine-stabilized ligand domains, and the like).
FKFL—Cathepsin B substrate for endosomal cleavage (bioresponsive domain may be customized for each cell/tissue/organ/cancer matrix metalloprotease [MMP] and/or other proteolytic enzymes (as detailed elsewhere).
FDIIKKIAES—Bioresponsive functional domain (ref: Discovery and Characterization of a Peptide That Enhances Endosomal Escape of Delivered Proteins in Vitro and in Vivo Margie Li, Yong Tao, Yilai Shu, Jonathan R. LaRochelle, Angela Steinauer, David Thompson, Alanna Schepartz, Zheng-Yi Chen, and David R. LiuJournal of the American Chemical Society 2015 137 (44), 14084-14093 DOI: 10.1021/jacs.5b05694). In this case a helical domain serves an endosomal escape function, however this particular peptide may have additional utility as well (FIG. 18A depicts a multifunctional peptide sequence which includes aurein 1.2, an antimicrobial and anticancer peptide from an Australian frog, which represents an endosomolytic/helical/spacer domain with optional cleavage domain (e.g. FKFL or protease cleavage site) with a subsequent display of an optional ligand for cellular receptor affinity (see: https://www.rcsb.org/structure/1VM5).

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12. In some cases, a targeting ligand includes the amino acid sequence RGD and/or the amino acid sequence set forth in any one of SEQ ID NOs: 1-12. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12.

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12 and 181-187. In some cases, a targeting ligand includes the amino acid sequence RGD and/or the amino acid sequence set forth in any one of SEQ ID NOs: 1-12 and 181-187. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12 and 181-187.

A targeting ligand (e.g., of a delivery molecule) can include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12, 181-187, and 271-277. In some cases, a targeting ligand includes the amino acid sequence RGD and/or the amino acid sequence set forth in any one of SEQ ID NOs: 1-12, 181-187, and 271-277. In some embodiments, a targeting ligand can include a cysteine (internal, C-terminal, or N-terminal), and can also include the amino acid sequence RGD and/or an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 1-12, 181-187, and 271-277.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187, and 271-277. In some cases, a targeting ligand includes the amino acid sequence set forth in any one of SEQ ID NOs: 181-187, and 271-277. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187, and 271-277.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187. In some cases, a targeting ligand includes the amino acid sequence set forth in any one of SEQ ID NOs: 181-187. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 181-187.

In some cases, a targeting ligand (e.g., of a delivery molecule) can include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 271-277. In some cases, a targeting ligand includes the amino acid sequence set forth in any one of SEQ ID NOs: 271-277. In some embodiments, a targeting ligand can include a cysteine (internal, C-terminal, or N-terminal), and can also include an amino acid sequence having 85% or more sequence identity (e.g., 90% or more, 95% or more, 97% or more, 98% or more, 99% or more, 99.5% or more, or 100% sequence identity) with the amino acid sequence set forth in any one of SEQ ID NOs: 271-277.

The terms “targets” and “targeted binding” are used herein to refer to specific binding. The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, a ligand specifically binds to a particular receptor relative to other available receptors). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity correlates with a lower Kₐ.

In some cases, the targeting ligand provides for targeted binding to a cell surface protein selected from a family B G-protein coupled receptor (GPCR), a receptor tyrosine kinase (RTK), a cell surface glycoprotein, and a cell-cell adhesion molecule. Consideration of a ligand's spatial arrangement upon receptor docking can be used to accomplish a desired functional selectivity and endosomal sorting biases, e.g., so that the structure function relationship between the ligand and the target is not disrupted due to the conjugation of the targeting ligand to the payload or anchoring domain (e.g., cationic anchoring domain). For example, conjugation to a nucleic acid, protein, ribonucleoprotein, or anchoring domain (e.g., cationic anchoring domain) could potentially interfere with the binding cleft(s).

Thus, in some cases, where a crystal structure of a desired target (cell surface protein) bound to its ligand is available (or where such a structure is available for a related protein), one can use 3D structure modeling and sequence threading to visualize sites of interaction between the ligand and the target. This can facilitate, e.g., selection of internal sites for placement of substitutions and/or insertions (e.g., of a cysteine residue).

As an example, in some cases, the targeting ligand provides for binding to a family B G protein coupled receptor (GPCR) (also known as the ‘secretin-family’). In some cases, the targeting ligand provides for binding to both an allosteric-affinity domain and an orthosteric domain of the family B GPCR to provide for the targeted binding and the engagement of long endosomal recycling pathways, respectively (e.g., see FIGS. 10A-G).

G-protein-coupled receptors (GPCRs) share a common molecular architecture (with seven putative transmembrane segments) and a common signaling mechanism, in that they interact with G proteins (heterotrimeric GTPases) to regulate the synthesis of intracellular second messengers such as cyclic AMP, inositol phosphates, diacylglycerol and calcium ions. Family B (the secretin-receptor family or ‘family 2’) of the GPCRs is a small but structurally and functionally diverse group of proteins that includes receptors for polypeptide hormones and molecules thought to mediate intercellular interactions at the plasma membrane (see e.g., Harmar et al., Genome Biol. 2001; 2(12): REVIEWS3013). There have been important advances in structural biology as relates to members of the secretin-receptor family, including the publication of several crystal structures of their N-termini, with or without bound ligands, which work has expanded the understanding of ligand binding and provides a useful platform for structure-based ligand design (see e.g., Poyner et al., Br J Pharmacol. 2012 May; 166(1):1-3).

For example, one may desire to use a subject delivery molecule to target the pancreatic cell surface protein GLP1R (e.g., to target B-islets) using the Exendin-4 ligand, or a derivative thereof (e.g., a cysteine substituted Exendin-4 targeting ligand such as that presented as SEQ ID NO: 2). Because GLP1R is abundant within the brain and pancreas, a targeting ligand that provides for targeting binding to GLP1R can be used to target the brain and pancreas. Thus, targeting GLP1R facilitates methods (e.g., treatment methods) focused on treating diseases (e.g., via delivery of one or more gene editing tools) such as Huntington's disease (CAG repeat expansion mutations), Parkinson's disease (LRRK2 mutations), ALS (SOD1 mutations), and other CNS diseases. Targeting GLP1R also facilitates methods (e.g., treatment methods) focused on delivering a payload to pancreatic β-islets for the treatment of diseases such as diabetes mellitus type I, diabetes mellitus type II, and pancreatic cancer (e.g., via delivery of one or more gene editing tools).

When targeting GLP1R using a modified version of exendin-4, an amino acid for cysteine substitution and/or insertion (e.g., for conjugation to a nucleic acid payload) can be identified by aligning the Exendin-4 amino acid sequence, which is HGEGTFTSDLSKQMEEEAVRLFIEWLKNGGPSSGAPPPS

(SEQ ID NO. 1), to crystal structures of glucagon-GCGR (4ERS) and GLP1-GLP1R-ECD complex (PDB: 3IOL, using PDB 3 dimensional renderings, which may be rotated in 3D space in order to anticipate the direction that a cross-linked complex must face in order not to disrupt the two binding clefts. When a desirable cross-linking site (e.g., site for substitution/insertion of a cysteine residue) of a targeting ligand (that targets a family B GPCR) is sufficiently orthogonal to the two binding clefts of the corresponding receptor, high-affinity binding may occur as well as concomitant long endosomal recycling pathway sequestration (e.g., for improved payload release). The cysteine substitution at amino acid positions 10, 11, and/or 12 of SEQ ID NO: 1 confers bimodal binding and specific initiation of a Gs-biased signaling cascade, engagement of beta arrestin, and receptor dissociation from the actin cytoskeleton. In some cases, this targeting ligand triggers internalization of the nanoparticle via receptor-mediated endocytosis, a mechanism that is not engaged via mere binding to the GPCR's N-terminal domain without concomitant orthosteric site engagement (as is the case with mere binding of the affinity strand, Exendin-4 [31-39]).

In some cases, a subject targeting ligand includes an amino acid sequence having 85% or more (e.g., 90% or more, 95% or more, 98% or more, 99% or more, or 100%) identity to the exendin-4 amino acid sequence (SEQ ID NO: 1). In some such cases, the targeting ligand includes a cysteine substitution or insertion at one or more of positions corresponding to L10, S11, and K12 of the amino acid sequence set forth in SEQ ID NO: 1. In some cases, the targeting ligand includes a cysteine substitution or insertion at a position corresponding to S11 of the amino acid sequence set forth in SEQ ID NO: 1. In some cases, a subject targeting ligand includes an amino acid sequence having the exendin-4 amino acid sequence (SEQ ID NO: 1). In some cases, the targeting ligand is conjugated (with or without a linker) to an anchoring domain (e.g., a cationic anchoring domain).

As another example, in some cases a targeting ligand according to the present disclosure provides for binding to a receptor tyrosine kinase (RTK) such as fibroblast growth factor (FGF) receptor (FGFR). Thus in some cases the targeting ligand is a fragment of an FGF (i.e., comprises an amino acid sequence of an FGF). In some cases, the targeting ligand binds to a segment of the RTK that is occupied during orthosteric binding (e.g., see the examples section below). In some cases, the targeting ligand binds to a heparin-affinity domain of the RTK. In some cases, the targeting ligand provides for targeted binding to an FGF receptor and comprises an amino acid sequence having 85% or more sequence identity (e.g., 90% or more, 95% or more, 97% or more, 98% or more, 99% or more, 99.5% or more, or 100% sequence identity) with the amino acid sequence KNGGFFLRIHPDGRVDGVREKS (SEQ ID NO: 4). In some cases, the targeting ligand provides for targeted binding to an FGF receptor and comprises the amino acid sequence set forth as SEQ ID NO: 4.

In some cases, small domains (e.g., 5-40 amino acids in length) that occupy the orthosteric site of the RTK may be used to engage endocytotic pathways relating to nuclear sorting of the RTK (e.g., FGFR) without engagement of cell-proliferative and proto-oncogenic signaling cascades, which can be endemic to the natural growth factor ligands. For example, the truncated bFGF (tbFGF) peptide (a.a.30-115), contains a bFGF receptor binding site and a part of a heparin-binding site, and this peptide can effectively bind to FGFRs on a cell surface, without stimulating cell proliferation. The sequences of tbFGF are KRLYCKNGGFFLRIHPDGRVDGVREKSDPHIKLQLQAEERGVVSIKGVCANRYLAMKEDGRLLASK CVTDECFFFERLESNNYNTY (SEQ ID NO: 13) (see, e.g., Cai et al., Int J Pharm. 2011 Apr. 15; 408(1-2): 173-82).

In some cases, the targeting ligand provides for targeted binding to an FGF receptor and comprises the amino acid sequence HFKDPK (SEQ ID NO: 5) (see, e.g., the examples section below). In some cases, the targeting ligand provides for targeted binding to an FGF receptor, and comprises the amino acid sequence LESNNYNT (SEQ ID NO: 6) (see, e.g., the examples section below).

In some cases, a targeting ligand according to the present disclosure provides for targeted binding to a cell surface glycoprotein. In some cases, the targeting ligand provides for targeted binding to a cell-cell adhesion molecule. For example, in some cases, the targeting ligand provides for targeted binding to CD34, which is a cell surface glycoprotein that functions as a cell-cell adhesion factor, and which is protein found on hematopoietic stem cells (e.g., of the bone marrow). In some cases, the targeting ligand is a fragment of a selectin such as E-selectin, L-selectin, or P-selectin (e.g., a signal peptide found in the first 40 amino acids of a selectin). In some cases a subject targeting ligand includes sushi domains of a selectin (e.g., E-selectin, L-selectin, P-selectin).

In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIASQFLSALTLVLLIKESGA (SEQ ID NO: 7). In some cases, the targeting ligand comprises the amino acid sequence set forth as SEQ ID NO: 7. In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MVFPWRCEGTYWGSRNILKLWVWTLLCCDFLIHHGTHC (SEQ ID NO: 8). In some cases, the targeting ligand comprises the amino acid sequence set forth as SEQ ID NO: 8. In some cases, targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIFPWKCQSTQRDLWNIFKLWGWTMLCCDFLAHHGTDC (SEQ ID NO: 9). In some cases, targeting ligand comprises the amino acid sequence set forth as SEQ ID NO: 9. In some cases, targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence MIFPWKCQSTQRDLWNIFKLWGWTMLCC (SEQ ID NO: 10). In some cases, targeting ligand comprises the amino acid sequence set forth as SEQ ID NO: 10.

Fragments of selectins that can be used as a subject targeting ligand (e.g., a signal peptide found in the first 40 amino acids of a selectin) can in some cases attain strong binding to specifically-modified sialomucins, e.g., various Sialyl Lewis' modifications/O-sialylation of extracellular CD34 can lead to differential affinity for P-selectin, L-selectin and E-selectin to bone marrow, lymph, spleen and tonsillar compartments. Conversely, in some cases a targeting ligand can be an extracellular portion of CD34. In some such cases, modifications of sialylation of the ligand can be utilized to differentially target the targeting ligand to various selectins.

In some cases, a targeting ligand according to the present disclosure provides for targeted binding to E-selectin. E-selectin can mediate the adhesion of tumor cells to endothelial cells and ligands for E-selectin can play a role in cancer metastasis. As an example, P-selectin glycoprotein-1 (PSGL-1) (e.g., derived from human neutrophils) can function as a high-efficiency ligand for E-selectin (e.g., expressed by the endothelium), and a subject targeting ligand can therefore in some cases include the PSGL-1 amino acid sequence (or a fragment thereof the binds to E-selectin). As another example, E-selectin ligand-1 (ESL-1) can bind E-selectin and a subject targeting ligand can therefore in some cases include the ESL-1 amino acid sequence (or a fragment thereof the binds to E-selectin). In some cases, a targeting ligand with the PSGL-1 and/or ESL-1 amino acid sequence (or a fragment thereof the binds to E-selectin) bears one or more sialyl Lewis modifications in order to bind E-selectin. As another example, in some cases CD44, death receptor-3 (DR3), LAMP1, LAMP2, and Mac2-BP can bind E-selectin and a subject targeting ligand can therefore in some cases include the amino acid sequence (or a fragment thereof the binds to E-selectin) of any one of: CD44, death receptor-3 (DR3), LAMP1, LAMP2, and Mac2-BP.

In some cases, a targeting ligand according to the present disclosure provides for targeted binding to P-selectin. In some cases PSGL-1 can provide for such targeted binding. In some cases a subject targeting ligand can therefore in some cases include the PSGL-1 amino acid sequence (or a fragment thereof the binds to P-selectin). In some cases, a targeting ligand with the PSGL-1 amino acid sequence (or a fragment thereof the binds to P-selectin) bears one or more sialyl Lewis modifications in order to bind P-selectin.

In some cases, a targeting ligand according to the present disclosure provides for targeted binding to a target selected from: CD3, CD8, CD4, CD28, CD90, CD45f, CD34, CD80, CD86, CD19, CD20, CD22, CD47, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL2R, IL7R, IL10R, IL12R, IL15R, IL18R, TNFα, IFNγ, TGF-β, and α5β1

In some cases, a targeting ligand according to the present disclosure provides for targeted binding to a transferrin receptor. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence THRPPMWSPVWP (SEQ ID NO: 11). In some cases, targeting ligand comprises the amino acid sequence set forth as SEQ ID NO: 11.

In some cases, a targeting ligand according to the present disclosure provides for targeted binding to an integrin (e.g., α5β1 integrin). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence RRETAWA (SEQ ID NO: 12). In some cases, targeting ligand comprises the amino acid sequence set forth as SEQ ID NO: 12. In some cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence RGDGW (SEQ ID NO: 181). In some cases, targeting ligand comprises the amino acid sequence set forth as SEQ ID NO: 181. In some cases, the targeting ligand comprises the amino acid sequence RGD.

In some cases, a targeting ligand according to the present disclosure provides for targeted binding to an integrin. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence GCGYGRGDSPG (SEQ ID NO: 182). In some cases, the targeting ligand comprises the amino acid sequence set forth as SEQ ID NO: 182. In some cases such a targeting ligand is acetylated on the N-terminus and/or amidated (NH2) on the C-terminus.

In some cases, a targeting ligand according to the present disclosure provides for targeted binding to an integrin (e.g., a5133 integrin). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence DGARYCRGDCFDG (SEQ ID NO: 187). In some cases, the targeting ligand comprises the amino acid sequence set forth as SEQ ID NO: 187.

In some embodiments, a targeting ligand used to target the brain includes an amino acid sequence from rabies virus glycoprotein (RVG) (e.g., YTIWMPENPRPGTPCDIFTNSRGKRASNGGGG (SEQ ID NO: 183)). In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 183. As for any of targeting ligand (as described elsewhere herein), RVG can be conjugated and/or fused to an anchoring domain (e.g., 9R peptide sequence). For example, a subject delivery molecule used as part of a surface coat of a subject nanoparticle can include the sequence YTIWMPENPRPGTPCDIFTNSRGKRASNGGGGRRRRRRRRR (SEQ ID NO: 180).

In some cases, a targeting ligand according to the present disclosure provides for targeted binding to c-Kit receptor. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 184. In some cases, the targeting ligand comprises the amino acid sequence set forth as SEQ ID NO: 184.

In some cases, a targeting ligand according to the present disclosure provides for targeted binding to CD27. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 185. In some cases, the targeting ligand comprises the amino acid sequence set forth as SEQ ID NO: 185.

In some cases, a targeting ligand according to the present disclosure provides for targeted binding to CD150. In some such cases, the targeting ligand comprises an amino acid sequence having 85% or more sequence identity (e.g., 90% or more, 95% or more, 97% or more, 98% or more, 99% or more, 99.5% or more, or 100% sequence identity) with the amino acid sequence set forth as SEQ ID NO: 186. In some cases, the targeting ligand comprises the amino acid sequence set forth as SEQ ID NO: 186.

In some embodiments, a targeting ligand provides for targeted binding to KLS CD27+/IL-7Ra-/CD150+/CD34-hematopoietic stem and progenitor cells (HSPCs). For example, a gene editing tool(s) (described elsewhere herein) can be introduced in order to disrupt expression of a BCL11a transcription factor and consequently generate fetal hemoglobin. As another example, the beta-globin (HBB) gene may be targeted directly to correct the altered E7V substitution with a corresponding homology-directed repair donor DNA molecule. As one illustrative example, a CRISPR/Cas RNA-guided polypeptide (e.g., Cas9, CasX, CasY, Cpf1) can be delivered with an appropriate guide RNA such that it will bind to loci in the HBB gene and create double-stranded or single-stranded breaks in the genome, initiating genomic repair. In some cases, a Donor DNA molecule (single stranded or double stranded) is introduced (as part of a payload) and is release for 14-30 days while a guide RNA/CRISPR/Cas protein complex (a ribonucleoprotein complex) can be released over the course of from 1-7 days.

In some embodiments, a targeting ligand provides for targeted binding to CD4+ or CD8+ T-cells, hematopoietic stem and progenitor cells (HSPCs), or peripheral blood mononuclear cells (PBMCs), in order to modify the T-cell receptor. For example, a gene editing tool(s) (described elsewhere herein) can be introduced in order to modify the T-cell receptor. The T-cell receptor may be targeted directly and substituted with a corresponding homology-directed repair donor DNA molecule for a novel T-cell receptor. As one example, a CRISPR/Cas RNA-guided polypeptide (e.g., Cas9, CasX, CasY, Cpf1) can be delivered with an appropriate guide RNA such that it will bind to loci in the TCR gene and create double-stranded or single-stranded breaks in the genome, initiating genomic repair. In some cases, a Donor DNA molecule (single stranded or double stranded) is introduced (as part of a payload). It would be evident to skilled artisans that other CRISPR guide RNA and donor sequences, targeting beta-globin, CCR5, the T-cell receptor, or any other gene of interest, and/or other expression vectors may be employed in accordance with the present disclosure.

In some embodiments, a targeting ligand is a nucleic acid aptamer. In some embodiments, a targeting ligand is a peptoid.

Also provided are delivery molecules with two different peptide sequences that together constitute a targeting ligand. For example, in some cases a targeting ligand is bivalent (e.g., heterobivalent). In some cases, cell-penetrating peptides and/or heparin sulfate proteoglycan binding ligands are used as heterobivalent endocytotic triggers along with any of the targeting ligands of this disclosure. A heterobivalent targeting ligand can include an affinity sequence from one of targeting ligand and an orthosteric binding sequence (e.g., one known to engage a desired endocytic trafficking pathway) from a different targeting ligand.

In some cases, targeting ligands are identified by screening (also described in more detail elsewhere herein). The term “top-performing” targeting ligands can be used to mean the targeting ligands that perform best in the assays when comparted to other ligands of the screen. The criteria used to determine which ligands are “top-performing” can be any convenient criteria. Examples of such parameters can include physical and/or biological measures of performance. Examples can include transfection efficiency, cell specificity, etc. In some cases, the “top-performing” ligands are the top 50 (e.g., top 40, top 30, top 20, top 15, top 10, or top 5) performing ligands. In some cases, the “top-performing” ligands are the top 30 (e.g., top 20, top 15, top 10, or top 5) performing ligands. In some cases, the “top-performing” ligands are the top 15, e.g., top 10 or top 5) performing ligands. In some cases, the “top-performing” ligands are the top performing 20% of ligands (e.g., top 10% or top 5%) (e.g., if 1000 ligands were screened, the top-performing 20% would be the top 200 performing 200). In some cases, the “top-performing” ligands are the top performing 10% of ligands (e.g., top 5% or top 2% or top 1%) (e.g., if 1000 ligands were screened, the top-performing 10% would be the top performing 100 ligands). In some cases, the “top-performing” ligands are the top performing 5% of ligands (e.g., top 2% or top 1%) (e.g., if 1000 ligands were screened, the top-performing 5% would be the top performing 50 ligands). In some cases, the “top-performing” ligands are the top performing 2% of ligands (e.g., top 1%) (e.g., if 1000 ligands were screened, the top-performing 2% would be the top performing 20 ligands).

Anchoring Domain

In some embodiments, a delivery molecule includes a targeting ligand conjugated to an anchoring domain (e.g., cationic anchoring domain, an anionic anchoring domain). In some cases a subject delivery vehicle includes a payload that is condensed with and/or interacts electrostatically or covalently with the anchoring domain (e.g., a delivery molecule can be the delivery vehicle used to deliver the payload). In some cases the surface coat of a nanoparticle includes such a delivery molecule with an anchoring domain, and in some such cases the payload is in the core (interacts with the core) of such a nanoparticle. In some cases, the payload is a small molecule or biologic covalently attached to anchoring domain. See the above section describing charged polymer polypeptide domains for additional details related to anchoring domains.

In some cases, an outer layer (surface layer) can include motifs that lend stealth functionality, limiting protein corona formation, and complement activity. These motifs may be composed of carbohydrate functionalized peptides, polysialic acid, hyaluronic acid, poly(ethylene glycol) or any other hydrated biopolymers.

Alternative Packaging (e.g., Lipid Formulations)

In some embodiments, a subject core (e.g., including any combination of components and/or configurations described above) is part of a lipid-based delivery system, e.g., a cationic lipid delivery system (see, e.g., Chesnoy and Huang, Annu Rev Biophys Biomol Struct. 2000, 29:27-47; Hirko et al., Curr Med Chem. 2003 Jul. 10(14):1185-93; and Liu et al., Curr Med Chem. 2003 Jul. 10(14):1307-15). In some cases a subject core (e.g., including any combination of components and/or configurations described above) is not surrounded by a sheddable layer. As noted above a core can include an anionic polymer composition (e.g., poly(glutamic acid)), a cationic polymer composition (e.g., poly(arginine), a cationic polypeptide composition (e.g., a histone tail peptide), and a payload (e.g., nucleic acid and/or protein payload).

In some cases in which the core is part of a lipid-based delivery system, the core was designed with timed and/or positional (e.g., environment-specific) release in mind. For example, in some cases the core includes ESPs, ENPs, and/or EPPs, and in some such cases these components are present at ratios such that payload release is delayed until a desired condition (e.g., cellular location, cellular condition such as pH, presence of a particular enzyme, and the like) is encountered by the core (e.g., described above). In some such embodiments the core includes polymers of D-isomers of an anionic amino acid and polymers of L-isomers of an anionic amino acid, and in some cases the polymers of D- and L-isomers are present, relative to one another, within a particular range of ratios (e.g., described above). In some cases the core includes polymers of D-isomers of a cationic amino acid and polymers of L-isomers of a cationic amino acid, and in some cases the polymers of D- and L-isomers are present, relative to one another, within a particular range of ratios (e.g., described above). In some cases the core includes polymers of D-isomers of an anionic amino acid and polymers of L-isomers of a cationic amino acid, and in some cases the polymers of D- and L-isomers are present, relative to one another, within a particular range of ratios (e.g., described above). In some cases the core includes polymers of L-isomers of an anionic amino acid and polymers of D-isomers of a cationic amino acid, and in some cases the polymers of D- and L-isomers are present, relative to one another, within a particular range of ratios (e.g., described elsewhere herein). In some cases the core includes a protein that includes an NLS (e.g., described elsewhere herein). In some cases the core includes an HTP (e.g., described elsewhere herein).

Cationic lipids are nonviral vectors that can be used for gene delivery and have the ability to condense plasmid DNA. After synthesis of N-[1-(2,3-dioleyloxy)propyl]-N,N,N-trimethylammonium chloride for lipofection, improving molecular structures of cationic lipids has been an active area, including head group, linker, and hydrophobic domain modifications. Modifications have included the use of multivalent polyamines, which can improve DNA binding and delivery via enhanced surface charge density, and the use of sterol-based hydrophobic groups such as 3B-[N—(N′,N′-dimethylaminoethane)-carbamoyll cholesterol, which can limit toxicity. Helper lipids such as dioleoyl phosphatidylethanolamine (DOPE) can be used to improve transgene expression via enhanced liposomal hydrophobicity and hexagonal inverted-phase transition to facilitate endosomal escape. In some cases a lipid formulation includes one or more of: DLin-DMA, DLin-K-DMA, DLin-KC2-DMA, DLin-MC3-DMA, 98N12-5, C12-200, a cholesterol a PEG-lipid, a lipidopolyamine, dexamethasone-spermine (DS), and disubstituted spermine (D2S) (e.g., resulting from the conjugation of dexamethasone to polyamine spermine). DLin-DMA, DLin-K-DMA, DLin-KC2-DMA, 98N12-5, C12-200 and DLin-MC3-DMA can be synthesized by methods outlined in the art (see, e.g, Heyes et. al, J. Control Release, 2005, 107, 276-287; Semple et. al, Nature Biotechnology, 2010, 28, 172-176; Akinc et. al, Nature Biotechnology, 2008, 26, 561-569; Love et. al, PNAS, 2010, 107, 1864-1869; international patent application publication WO2010054401; all of which are hereby incorporated by reference in their entirety.

Examples of various lipid-based delivery systems include, but are not limited to those described in the following publications: international patent publication No. WO2016081029; U.S. patent application publication Nos. US20160263047 and US20160237455; and U.S. Pat. Nos. 9,533,047; 9,504,747; 9,504,651; 9,486,538; 9,393,200; 9,326,940; 9,315,828; and 9,308,267; all of which are hereby incorporated by reference in their entirety.

As such, in some cases a subject core is surrounded by a lipid (e.g., a cationic lipid such as a LIPOFECTAMINE transfection reagent). In some cases a subject core is present in a lipid formulation (e.g., a lipid nanoparticle formulation). A lipid formulation can include a liposome and/or a lipoplex. A lipid formulation can include a Spontaneous Vesicle Formation by Ethanol Dilution (SNALP) liposome (e.g., one that includes cationic lipids together with neutral helper lipids which can be coated with polyethylene glycol (PEG) and/or protamine).

A lipid formulation can be a lipidoid-based formulation. The synthesis of lipidoids has been extensively described and formulations containing these compounds can be included in a subject lipid formulation (see, e.g., Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; and Siegwart et al., Proc Natl Acad Sci USA. 2011 108:12996-3001; all of which are incorporated herein by reference in their entirety). In some cases a subject lipid formulation can include one or more of (in any desired combination): 1,2-Dioleoyl-sn-glycero-3-phosphatidylcholine (DOPC); 1,2-Dioleoyl-sn-glycero-3-phosphatidylethanolamine (DOPE); N-[1-(2,3-Dioleyloxy)prophyl]N,N,N-trimethylammonium chloride (DOTMA); 1,2-Dioleoyloxy-3-trimethylammonium-propane (DOTAP); Dioctadecylamidoglycylspermine (DOGS); N-(3-Aminopropyl)-N,N-dimethyl-2,3-bis(dodecyloxy)-1 (GAP-DLRIE); propanaminium bromide; cetyltrimethylammonium bromide (CTAB); 6-Lauroxyhexyl ornithinate (LHON); 1-(2,3-Dioleoyloxypropyl)-2,4,6-trimethylpyridinium (20c); 2,3-Dioleyloxy-N-[2(sperminecarboxamido-ethyl]-N,N-dimethyl-1 (DOSPA); propanaminium trifluoroacetate; 1,2-Dioleyl-3-trimethylammonium-propane (DOPA); N-(2-Hydroxyethyl)-N,N-dimethyl-2,3-bis(tetradecyloxy)-1 (MDRIE); propanaminium bromide; dimyristooxypropyl dimethyl hydroxyethyl ammonium bromide (DMRI); 3.beta.-[N—(N′,N′-Dimethylaminoethane)-carbamoyl]cholesterol DC-Chol; bis-guanidium-tren-cholesterol (BGTC); 1,3-Diodeoxy-2-(6-carboxy-spermyl)-propylamide (DOSPER); Dimethyloctadecylammonium bromide (DDAB); Dioctadecylamidoglicylspermidin (DSL); rac-[(2,3-Dioctadecyloxypropyl)(2-hydroxyethyl)]-dimethylammonium (CLIP-1); chloride rac-[2(2,3-Dihexadecyloxypropyl (CLIP-6); oxymethyloxy)ethyl]trimethylammonium bromide; ethyldimyristoylphosphatidylcholine (EDMPC); 1,2-Distearyloxy-N,N-dimethyl-3-aminopropane (DSDMA); 1,2-Dimyristoyl-trimethylammonium propane (DMTAP); O,O′-Dimyristyl-N-lysyl aspartate (DMKE); 1,2-Distearoyl-sn-glycero-3-ethylphosphocholine (DSEPC); N-Palmitoyl D-erythro-sphingosyl carbamoyl-spermine (CCS); N-t-Butyl-N0-tetradecyl-3-tetradecylaminopropionamidine; diC14-amidine; octadecenolyoxy[ethyl-2-heptadecenyl-3 hydroxyethyl] imidazolinium (DOTIM); chloride N1-Cholesteryloxycarbonyl-3,7-diazanonane-1,9-diamine (CDAN); 243-[bis(3-aminopropyl)amino]propylaminol-N-[2-[di(tetradecl]amino]-2-oxoethyl]acetamide (RPR209120); ditetradecylcarbamoylme-ethyl-acetamide; 1,2-dilinoleyloxy-3-dimethylaminopropane (DLinDMA); 2,2-dilinoleyl-4-dimethylaminoethyl-[1,3]-dioxolane; DLin-KC2-DMA; dilinoleyl-methyl-4-dimethylaminobutyrate; DLin-MC3-DMA; DLin-K-DMA; 98N12-5; C12-200; a cholesterol; a PEG-lipid; a lipiopolyamine; dexamethasone-spermine (DS); and disubstituted spermine (D25).

### Personalized/Diagnostically-Responsive Methods and Compositions

As noted above, in some cases methods and compositions of the disclosure can be diagnostically responsive (i.e., designed based on information such as RNA and/or protein expression data from the individual being treated). As such, design of the delivery vehicle (e.g., selection of an appropriate nanoparticle targeting ligand) and/or payload (e.g., choice of a particular promoter for expressing a heterologous RNA and/or protein) can be tailored to the specific characteristics of a patient's disease. This may be accomplished in a diagnostically responsive manner, e.g., after biopsy and analysis of the retrieved tissue/cells.

In some cases, the information used from an individual when designing a diagnostically responsive formulation is information from high throughput methodologies such as high throughput/next generation RNA or DNA sequencing methods (e.g., nanopore sequencing, 454 pyrophosphate sequencing, single molecule Heliscope sequencing, nano-array sequencing, SOLiD sequencing, Illumina/Solexa sequencing, Ion Torrent sequencing, Single-molecule real-time (SMRT) sequencing, and the like—see, e.g., Reuter et al., Mol Cell. 2015 May 21; 58(4):586-97). In some cases, the information used from an individual when designing a diagnostically responsive formulation is information from high throughput proteomic technologies (e.g., Mass spectrometry (MS)-based high-throughput proteomics, antibody arrays, peptide arrays, ligand/receptor-based arrays, and the like—see, e.g., Zhang et al., Annu Rev Anal Chem (Palo Alto Calif.). 2014; 7:427-54; Paczesny et al., Proteomics Clin Appl. 2018 Oct. 11:e1800145). In some cases, the information used is the identity of (e.g., a list of) proteins and/or nucleic acids that are highly expressed, enriched, and/or specifically expressed in diseased tissue such as cancer cells. In some such cases, the information used includes or is even limited to cell surface proteins that are highly expressed, enriched, and/or specifically expressed in diseased tissue such as cancer cells.

While the information used from an individual can be from high throughput methodologies, such information is not necessary in all cases. For example, in some cases, a disease such as a particular type of cancer can classified into subgroupings based on previously determined diagnostic assays. In some cases, such assays can be used to identify a desired protein and/or nucleic acid (e.g., a surface protein) that is highly expressed, enriched, and/or specifically expressed in diseased tissue such as cancer cells.

The information used from an individual can in some cases include identification of one or more of: (1) highly expressed, enriched, and/or specifically expressed surface protein(s) (e.g., receptors); (2) a promoter(s) that is highly expressed, enriched, and/or specifically expressed; and (3) highly expressed, enriched, and/or specifically expressed proteolytic enzyme(s) (e.g. MMPs, cathepsins).

A subject delivery vehicle such as a nanoparticle and/or payload can then be designed based on the individual's information (e.g., diagnosis/classification, based on an identified enriched surface protein in a target cell/tissue/organ). As examples:
(1) when the information from the individual includes the identification of surface protein(s), a targeting ligand can be designed for use with a subject delivery vehicle, where the targeting ligand includes a peptide, antibody, antibody fragment, aptamer, or other targeting molecule that targets/binds to the identified enriched/specific surface protein—and in that way a payload can be targeted to diseased tissue of the individual;
(2) when the information from the individual includes the identification of a promoter that is active in diseased tissue (e.g., a promoter that highly expressed, enriched, and/or specifically utilized in disease tissue such as cancer tissue), a payload can be designed for use with a subject delivery vehicle, where the payload includes a desired gene operably linked to (i.e., under the control of) the identified promoter (or miRNAs, other conditional genetic expression/suppression approaches, and/or other forms of genetic AND/OR gates such as conditional siRNAs, synthetic biological circuits, and the like)—and in that way a payload can be delivered where a desired gene is expressed or edited only by the targeted disease tissues. In some cases, the desired gene that is placed under the control of the identified promoter is an affinity marker (described in more detail below), e.g., one in which a membrane anchored region (e.g., a transmembrane domain) is fused to an extracellular portion that elicits an immune response and optional intracellular signaling domain to modulate immune responsiveness, e.g. secretion of interleukins to create a “hot” tumor microenvironment; and
(3) when the information from the individual includes the identification of highly expressed, enriched, and/or specifically expressed proteolytic enzyme(s) or other cell-specific substrate(s) (e.g. histone-tail peptides with modifications leading to payload release in specific cells/tissues), nanoparticle architecture can be designed to include polypeptide or payloads sequences that are targets for the identified proteolytic enzymes or other substrates—and in that way a delivery vehicle (e.g., nanoparticle) can be delivered in which the payload is not fully released unless the delivery vehicle is in the presence of the desired environment (e.g., diseased tissue that produces the identified proteolytic enzyme), or whereby a released payload retains cell-specific expression/editing patterns.

### Illustrative Examples of the Above

A novel approach for modeling and predicting ideal target sequences in a desired cell, tissue, organ or cancer target is outlined whereby a database containing RNAseq and/or proteomics data is compared against expression patterns in all available datasets for healthy tissues. This allows for generating various means of establishing the selectivity of a given receptor/surface protein targeting approach. In this example, data was gathered from the GTEx portal and Human Protein Atlas.

| | |
|---|---|
| Inclusion | For sets X_{1, 2, 3, (. . .), p} |
| Criteria | Where “X” defines the consolidated dataset of top-expressed surface markers on each |
| X_{1, 2, 3, (. . .), p} | target cell population, for sets 1 − p, where p represents each therapeutically relevant cell |
| A_{1, 2, 3, (. . .), q} | subtype |
| γ_{1, 2, 3, (. . .), v} | Identify 5-50 most expressed surface markers on each target cell |
| | Useful for ex vivo or in vivo target identification |
| | For sets A_{1, 2, 3, (. . .), q} |
| | Where “A” defines the consolidated dataset of top-expressed surface markers on each |
| | physiologically-relevant organ target, for sets 1 − v, where v represents each therapeutically |
| | relevant organ |
| | Identify 5-50 most expressed surface markers on each target organ |
| | Useful for in vivo target identification |
| | Sets A and X may either be pooled for additional organ specificity of the targeting |
| | approach, or excluded such that only hyper-expressed proteins in BOTH sets A and X are |
| | further compared to the most expressed proteins in non-target cells, tissues and organs, γ |
| | represents the organ surface marker inclusion criteria, or the organ-by-organ inclusion |
| | index, γ is a form of inclusion criteria for finding surface markers on multiple organs that |
| | may overlap with the desired cell target population, as well as finding surface markers that |
| | are shared between organs prior to performing exclusion criteria by comparing target cells |
| | to off-target cells/tissues/organs. γ is designed to add organ-specific surface markers to the |
| | database by preventing negative sorting events for surface markers prior to identifying |
| | ideal biodistribution ligands for the shared organs. In other words, a set of ligands that |
| | achieves ideal biodistribution to the greatest number of specific cell type bearing organs |
| | (e.g. lymph nodes, bone marrow, blood, spleen, tonsils, appendix, etc. in the case of |
| | immunological targeting) may be used on its own (e.g. independently of cell-specific |
| | targeting ligands for the ultimate cell subpopulation being targeted because of the organ |
| | biodistribution created) or in combination with cell-specific targeting ligands for a desired |
| | cell subpopulation in order to confer optimal systemic biodistribution and balance between |
| | cell specificity and organ biodistribution. The inclusion of γ, which measures the |
| | differential expression between the greatest-expressing target organ containing a given |
| | cell subpopulation of interest (e.g. Naive CD8+ T cells) is the primary difference between |
| | the Tissue Selectivity Index (Σβ_{w}) and Organ Selectivity Index (Σβ_{z}), where the summed |
| | series represents the consolidation of datasets X and A with exclusion criteria (elimination |
| | of target genes from dataset) based on the expression of target genes from target Organ(s) |
| | (summed series of top genes in sets A) independently of expression by Target Cells |
| | (summed series of top genes in sets X). Whether or not “acceptable off-target organs” are |
| | included within the modeling of inclusion criteria determines whether a Tissue Selectivity |
| | Index or Organ Selectivity Index is used, and in the latter case γ. Cell Selectivity Index, |
| | Tissue Selectivity Index and Organ Selectivity Index are further defined below. |
| Exclusion | For sets α_{1, 2, 3, (. . .), u} |
| Criteria | Where “α” defines all top-expressed surface proteins on target cell type(s), X, measured in |
| Y_{1, 2, 3, (. . .), r} | transcripts per million (RPKM, FPKM or TPM), divided by transcripts per million in each |
| B_{1, 2, 3, (. . .), s} | non-target cell type, Y. Each of the below selectivity indices is intended to be compiled as |
| α_{1, 2, 3, (. . .), u} | a summation series of gene expression data for all top-expressed surface marker genes per |
| β_{1, 2, 3, (. . .), w} | cell type, tissue, and/or organ. |
| β_{1, 2, 3, (. . .), z} | Cell Selectivity Index (α) = Fold Gene/Protein Expression in Target Cell Types (X) vs. |
| | Next Highest-Expressing Cell in Sorting Algorithm (e.g. compare Naive CD8+ T cell to |
| | each subsequent T cell subpopulation, immune cell subpopulation, and target organ cell |
| | subpopulations to determine uniquely and/or differentially targetable surface markers, |
| | then rank selectivity indices for each target cell type and organ vs. non-target cell types |
| | and organs) |
| | For sets β_{1, 2, 3, (. . .), w} |
| | Where “β” defines all top-expressed surface proteins on target cell type(s), X, measured in |
| | transcripts per million (RPKM, FPKM or TPM), divided by transcripts per million in non- |
| | target cell cell type(s), Y, AND organ(s), B. |
| | Tissue Selectivity Index (β) = Fold Gene/Protein Expression in Target Cell Types (X) vs. |
| | Next Highest-Expressing in Off-Target Cells AND Organs in Sorting Algorithm (ΣY and |
| | ΣB), where the summed series represents only genes identified in cell-specific |
| | overexpression (ΣX) (e.g. compare Naive CD8+ T cell to each subsequent T cell |
| | subpopulation, immune cell subpopulation, and target organ cell subpopulations to |
| | determine uniquely and/or differentially targetable surface markers, then rank selectivity |
| | indices for each target cell type vs. non-target cell types and organs). |
| | For sets β_{1, 2, 3, (. . .), z} |
| | Where “β” defines all top-expressed surface proteins on target cell type(s), X, AND |
| | organ(s), A, measured in transcripts per million (RPKM, FPKM or TPM) and divided by |
| | transcripts per million in non-target cell cell type(s), Y, AND organ(s), B. This may |
| | further be compared to the Tissue Selectivity Index to balance cell-specific targeting |
| | approaches with optimal organ biodistribution approaches. |
| | Organ Selectivity Index (β) = Fold Gene/Protein Expression in Target Cell Types |
| | (summed series of top genes in sets X) AND Organ(s) (summed series of top genes in A |
| | containing Target Cells from Sets X) vs. Next Highest-Expressing Organ in Sorting |
| | Algorithm (summed series of top genes in off-target organ B) AND Next Highest- |
| | Expressing Cell Type in Sorting Algorithm (summed series of top genes in off-target cells |
| | Y). For example, Naive CD8+ T cells may be found in lymph nodes, spleen, tonsils, blood |
| | and bone marrow, therefore targeting ligands that confer specificity to as many of the |
| | “acceptable” target organs, even independently of T cell targeting (in the immunological |
| | use case examples), may be used individually or heterovalently with varied targeting |
| | ligands that attain high specificity for the target cell. In this example, homologous |
| | liver/pancreas/etc. expression (low Tissue/Organ Specificity Index) of overexpressed |
| | genes on a desired cell subpopulation serves as elimination criteria, as does expression of |
| | a given gene on another immunological cell type (e.g. CD4+ T cells, B cells, NK cells). |
| | This approach takes into consideration that T cells are not found in abundance within the |
| | liver, for example, but targeting affinity for the other target organs (e.g. lymph nodes, |
| | spleen, tonsils, blood and bone marrow) may be therapeutically relevant for those cell |
| | types (e.g. targeting a matrix protein not found in the target cell, but found in abundance in |
| | the target organs such as lymph nodes, spleen, tonsils, blood, bone marrow). Additionally, |
| | even a targeting approach that creates chemotaxis for an off-target cell type (e.g. B cell |
| | when targeting a T cell) may be coupled to additional approaches for achieving hyper- |
| | avidity of the target cell type (e.g. T cell), minimizing endocytosis when a B cell is |
| | targeted (e.g. through a receptor antagonist that prevents endocytotic uptake), and/or |
| | engineering nanomaterials and cell-specific promoters to have a high degree of T cell |
| | specificity (e.g. expressing delivered/edited genes under T cell specific promoters, |
| | degrading nanoparticles under specific subcellular microenvironments that are cell- |
| | specific, etc.). |

Table 2 details an approach for generating selectivity indices for a given cell, tissue, or organ. This is further illustrated in FIGS. 10C-10G.

TABLE 3
details an approach for generating selectivity indices for a given cell,
tissue, or organ. This is further illustrated in FIGS. 10C-10FG.

| | | Equations |
|---|---|---|
| | | Summed Sets in Matrix Plots - Sort by Highest |
| | | Expressing to Lowest for Each Comparison in |
| | | the Series). |
| | | Each value X, Y, A and B represents the relative |
| | | gene/protein expression of one cell/tissue/organ |
| Algorithm | Key | vs. another, for a series of |
| Function | Variables | genes/proteins sorted by amount of expression. |
| Cell Specificity Index (αᵤ) | X_{1,2,3,(...),p} Y_{1,2,3,(...),r} αᵤ | ${α}_{u} = \frac{{\sum }_{l}^{p} X}{{\sum }_{l}^{r} Y}$ |
| Tissue Specificity Index (β_{w}) | X_{1,2,3,(...),p} Y_{1,2,3,(...),r} B_{1,2,3,(...),s} β_{w} | ${β}_{w} = \frac{{\sum }_{l}^{p} X - {\sum }_{l}^{r} Y}{{\sum }_{l}^{s} B}$ |
| Organ Specificity Index (βz) | X_{1,2,3,(...),p} Y_{1,2,3,(...),r} A_{1,2,3,(...),q} B_{1,2,3,(...),s} β_{z} | ${β}_{z} = \frac{{\sum }_{l}^{p} X + {\sum }_{l}^{q} A}{{\sum }_{l}^{s} B + {\sum }_{l}^{r} Y}$ |

TABLE 4
details exemplary cancer-specific promoters as derived from corresponding
overexpressed genes in tissue mRNA expression studies.
Illustrative Unique Promoters

| Promoter | Tumor | promoter sequence |
|---|---|---|
| AFP | Hepatocellular | AGTTTGAGGAGAATATTTGTTATATTTGCAAAATAAAATAAG |
| | carcinoma | TTTGCAAGTTTTTTTTTTCTGCCCCAAAGAGCTCTGTGTCCTT |
| | | GAACATAAAA |
| | | TACAAATAACCGCTATGCTGTTAATTATTGGCAAATGTCCCA |
| | | TTTTCAACCTAAGGAAATACCATAAAGTAACAGATATACCA |
| | | ACAAAAGGTTACTAGTTAACAGGCATTGCCTGAAAAGAGTA |
| | | TAAAAGAATTTCAGCATGATTTTCCATATTGTGCTTCCACCA |
| | | CTGCCAATAACAC |
| CCKAR | Pancreatic cancer | ACCCAGGTACCTATGTTCAAAAGTGCCTCAATCCTAGTTAAC |
| | | AAGGGCAGAGACCACGAGAAACAACACTGTGTTTAGTAGCA |
| | | ACTTAACAACCAGCCAGCAGTTCTGTCCACACACACACCACC |
| | | GGGCATGGTTCCAAAGCTAAAAAGGCACTAATTGCTTTTCTA |
| | | TAAGGAGGTAGAACACAGTCCCTCCGTGTTCTTTAGGCCTGA |
| | | TGGTCTGCATTATCGGATCTGTTACCGTGTTAATTGTTCCTGT |
| | | CTCACACAGCCGGTTTGGGCTTTCTTCTGCATATGTCTGGGA |
| | | TGGTGACGGGTTCCTATATAGAGGAGTACTGGGGAAGCCTCT |
| | | GTGTGTGTGTGTGTGTCCGTGCATATGTACACATGTGTGTAA |
| | | AAAGCAGCCACACGCTGAGAATGGTTAACGGGTAGCCAGGC |
| | | TGTCTGTACTCAGGGCCCTAAGACTGGCCCAGGAAAGGGCC |
| | | GGGGGAGGTGGGGCGGGGTGAAGGTGGAGCGGGCTTGGCTT |
| | | GTGCTCACTGCCTTTTCCACAACAGGAGTACAAATGCTGGAG |
| | | TGAGTGAGGTGAACTCAAGTCGCCTTTAGGAATGGCTGAAA |
| | | AAGCCCACACCTGGAAATCACTCCCTCCCTGCTCCTCCACGG |
| | | CAGGTTGCATCTGCGAGACGCTTCGGTCATTAGAGGAATGA |
| | | GCCGGGAGTGAGCAA |
| | | TTCACCAGCTCTCCAGCACTTGGTGGAAAGCAGCAGGCAAcc |
| CEA | Epithelial cancers | GCCCTGGAGAGCATGGGGAGACCCGGGACCCTGCTGGGTTT |
| | | CTCTGTCACAAAGGAAAATAATCCCCCTGGTGTGACAGACCC |
| | | AAGGACAGAACACAGCAGAGGTCAGCACTGGGGAAGACAG |
| | | GTTGTCCTCCCAGGGGATGGGGGTCCATCCACCTTGCCGAAA |
| | | AGATTTGTCTGAGGAACTGAAAATAGAAGGGAAAAAAGAGG |
| | | AGGGACAAAAGAGGCAGAAATGAGAGGGGAGGGGACAGAG |
| | | GACACCTGAATAAAGACCACACCCATGACCCACGTGATGCT |
| | | GAGAAGTACTCCTGCCCTAGGAAGAGACTCAGGGCAGAGGG |
| | | AGGAAGGACAGCAGACCAGACAGTCACAGCAGCCTTGACAA |
| | | AACGTTCCTGGAACTCAAGCTCTTCTCCACAGAGGAGGACA |
| | | GAGCAGACAGCAGAGACC |
| c-erbB2 | Breast & pancreas | CTGCTCGGGAGGCTGAGGCAGGAGAATCACTTGAACCAGGG |
| | cancer | AGGCAGAGGTTGTGGTGAGCAGAGATCGCGCCATTGCTCTC |
| | | CAGCCTGGGCAACAAGAGCAAAAGTTCGTTTAAAAAAAAAA |
| | | AAAAGTCCTTTCGATGTGACTGTCTCCTCCCAAATTTGTAGA |
| | | CCCTCTTAAGATCATGCTTTTCAGATACTTCAAAGATTCCAG |
| | | AAGATATGCCCCGGGGGTCCTGGAAGCCACAAGGTAAACAC |
| | | AACACATCCCCCTCCTTGACTATCAATTTTACTAGAGGATGT |
| | | GGTGGGAAAACCATTATTTGATATTAAAACAAATAGGCTTG |
| | | GGATGGAGTAGGATGCAAGCTCCCCAGGAAAGTTTAAGATA |
| | | AAACCTGAGACTTAAAAGGGTGTTAAGAGTGGCAGCCTAGG |
| | | GAATTTATCCCGGACTCCGGGGGAGGGGGCAGAGTCACCAG |
| | | CCTCTGCATTTAGGGATTCTCCGAGGAAAAGTGTGAGAACG |
| | | GCTGCAGGCAACCCAGGCGTCCCGGCGCTAGGAGGGACGCA |
| | | CCCAGGCCTGCGCGAAGAGAGGGAGAAAGTGAAGCTGGGA |
| | | GTTGCCACTCCCAGACTTGTTGGAATGCAGTTGGAGGGGGCG |
| | | AGCTGGGAGCGCGCTTGCTCCCAATCACAGGAGAAGGAGGA |
| | | GGTGGAGGAGGAGGGCTGCTTGAGGAAGTATAAGAATGAAG |
| | | TTGTGAAGCTGAGATTCCCCTCCATTGGGACCGGAGAAACCA |
| | | GGGGAGCCCCCCGGGCAGCCGCGCGCCCCTTCCCACGGGGC |
| | | CCTTTACTGCGCCGCGCGCCCGGCCCCCACCCCTCGCAGCAC |
| | | CCCGCGCCCCGCGCCCTCCCAGCCGGGTCCAGCCGGAGCCGT |
| | | GGGGCCGGAGCCGCAGTGAGCACC |
| MUC1 | Carcinoma cells | aCTAGtGITCATCGGAGCCCAGGTTTACTCCCTTAAGTGGAAA |
| | | TTTCTTCCCCCACTCCCTCCTTGGCTTTCTCCAAGGAGGGAAC |
| | | CCAGGCTACTGGAAAGTCCGGCTGGGGCGGGGACTGTGGGT |
| | | TTCAGGGTAGAACTGCGTGTGGAACGGGACAGGGAGCGGTT |
| | | AGAAGGGTGGGGCTATTCCGGGAAGTGGTGGGGGGAGGGA |
| | | GCCCAAAACTAGCACCTAGTCCACTCATTATCCAGCCCTCTT |
| | | ATTTCTCGGCCCCGCTCTGCTTCAGTGGACCCGGGGAGGGCG |
| | | GGGAAGTGGAGTGGGAGACCTAGGGGTGGGCTTCCCGACCT |
| | | TGCTGTACAGGACCTCGACCTAGCTGGCTTTGTTCCCCATCC |
| | | CCACGTTAGTTGTTGCCCTGAGGCTAAAACTAGAGCCCAGGG |
| | | GCCCCAAGTTCCAGACTGCCCCTCCCCCCTCCCCCGGAGCCA |
| | | GGGAGTGGTTGGTGAAAGGGGGAGGCCAGCTGGAGAACAA |
| | | ACGGGTAGTCAGGGGGTTGAGCGATTAGAGCCCTTGTACCCT |
| | | ACCCAGGAATGGTTGGGGAGGAGGAGGAAGAGGTAGGAGG |
| | | TAGGGGAGGGGGCGGGGTTTTGTCACCTGTCACCTGCTCCGG |
| | | CTGTGCCTAGGGCGGGCGGGCGGGGAGTGGGGGGACCGGTA |
| | | TAAAGCGGTAGGCGCCTGTGCCCGCTCCACCTCTCAAGCAGC |
| | | CAGCGCCTGCCTGAATCTGTTCTGCCCCCTCCCCACCCATTTC |
| | | ACCACCACC |
| PSA | Prostate and | ctagtACATTGTTTGCTGCACGTTGGATTTTGAAATGCTAGGGA |
| | prostate cancers | ACTTTGGGAGACTCATATTTCTGGGCTAGAGGATCTGTGGAC |
| | | CACAAG |
| | | ATCTTTTTATGATGACAGTAGCAATGTATCTGTGGAGCTGGA |
| | | TTCTGGGTTGGGAGTGCAAGGAAAAGAATGTACTAAATGCC |
| | | AAGACATCTATTTCAGGAGCATGAGGAATAAAAGTTCTAGTT |
| | | TCTGGTCTCAGAGTGGTGCAGGGATCAGGGAGTCTCACAATC |
| | | TCCTGAGTGCTGGTGTCTTAGGGCACACTGGGTCTTGGAGTG |
| | | CAAAGGATCTAGGCACGTGAGGCTTTGTATGAAGAATCGGG |
| | | GATCGTACCCACCCCCTGTTTCTGTTTCATCCTGGGCGTGTCT |
| | | CCTCTGCCTTTGTCCCCTAGATGAAGTCTCCATGAGCTACAG |
| | | GGCCTGGTGCATCCAGGGTGATCTAGTAATTGCAGAACAGC |
| | | AAGTGCTAGCTCTCCCTCCCCTTCCACAGCTCTGGGTGTGGG |
| | | AG |
| | | GGGGTTGTCCAGCCTCCAGCAGCATGGGGAGGGCCTTGGTC |
| | | AGCCTCTGGGTGCCAGCAGGGCAGGGGCGGAGTCCTGGGGA |
| | | ATGAAGGTTTTATAGGGCTCCTGGGGGAGGCTCCCCAGCCCC |
| | | AAGCTTACCACCTGCACCCGGAGAGCTGTGTCAC |
| TRP1 | Melanocytes & | CAGGCTTACATTTGAATGTTGGCTACATATGTATGAGTTTTC |
| | melanoma | AACTTCCAGGAGAAAACGTCTCTTTAAAAGAGAACAACCAA |
| | | AAGCTAACAGAAAATACAAGTGTGACATTGGCCTTAGTTCG |
| | | ACCAAGAAAGCAATTCATCTTGTTTCTTCCTTTGTGGTATAC |
| | | AGATAAAGAAAAATAAAATCACTACAACGAAAGCAAAATCT |
| | | CTTCAGCGTCTCTAATACATCTTCCAAATCAGTGTGTCTGAC |
| | | CTTTTCTTAAGACTTTAACCATCACAAGGAAACCAGTGGGGA |
| | | GGGAGTCATGTGCTGCCTAGTAGTTAAAGGGCAGGAGAATT |
| | | CACTGGTGTGAGAAGGGATTAGTGAGAGCTGGAAGAGAGGA |
| | | CCAGCCCCTCCCAGTGTGAGGAATCTGGCTTGGGATTTACTG |
| | | TCTGGCAGAAAATCTCTTCGGGCAATTAACAGCTGGCATCAG |
| | | GGGAAAAGCAGACATCCAACAACACTAGCTCTGAAGGAGAT |
| | | CAGCAGAGAAACCTTCCAGGGATTCATGGTACTGGTGAGCA |
| | | GCTCTGTGGTGGGTACCCTGTGACCAAAGCTCTAGGAACATG |
| | | AAGGAGATTTGCTTGCTATAAACCTGTTTCCTATTCTCCTTTC |
| | | ATTTCCATGGTTAACTATTACTATGGTAGTCACCAACTAGTG |
| | | GATGCTTTTGGTAAATGACATCTATGGAAAGTCTTTTTGGAT |
| | | CAGGGTGATCTTTTTATGTATGTGTATGTGCATGGATATGGG |
| | | TGCACGAGAGCAGGTGCCCAGATTCTCAAGGAGGGCTTCAG |
| | | TTACAAGGAGTTGGGAGTGATCTGATGTGGTTGCAAGGCACT |
| | | GAAGTCAGTCTCTCTGTAAGAGCACTCTATGCTCCTTACCAC |
| | | TGTGCCTTCTCCCCAGCCCAAGAATAGTATTCTTATGGGTAG |
| | | AAATTTTAAATAAGAAAACTCAAAGACCAGGAGAGTGAGTT |
| | | CTGTCATCTAGCTATTATGCCTGCAGATATTTAAAGGTGAAT |
| | | AATTGTTTTGACTATTGTTTAGAAATGTTGTTTCACATGAAA |
| | | GATTCCATTTCCGGAGTGGGTTGAAAAAGTATGCAAAAGAA |
| | | CTTTTGCAACTCTGTTTTTGCCTTTCTGTTTTTCAGCTGTATTT |
| | | TCATCTGAGCACCCCTGTCTTCTCCATGCAAAGAGCAGCATA |
| | | GGAGACCTGTGTTCTGAACTCTTGCTTCGAGAAC |
| Tpr | Melanocytes & | GACCTTTATTCATAAGAGATGATGTATTCTTGATACTACTTCT |
| | melanoma | CATTTGCAAATTCCAATTATTATTAATTTCATATCAATTAGAA |
| | | TAATATATCTTC |
| | | CTTCAATTTAGTTACCTCACTATGGGCTATGTACAAACTCCA |
| | | AGAAAAAGTTAGTCATGTGCTTTGCAGAAGATAAAAGCTTA |
| | | GTGTAAAACAGGCTGAGAGTATTTGATGTAAGAAGGGGAGT |
| | | GGTTATATAGGTCTTAGCCAAAACATGTGATAGTCACTCCAG |
| | | GGGTTGCTGGAAAAGAAGTCTGTGACACTCATTAACCTATTG |
| | | GTGCAGAATTTGAATGATCTAAAGGAGACC |

TABLE 5
depicts exemplary T cell and HSC cell-specific promoters derived
from overexpressed genes in the given cell population. Genes with
high cell/tissue/organ-specificity indices can have their
associated promoters utilized as additional tools for achieving
cell/tissue/organ-specific expression.

| Gene | Promoter | Sequence |
|---|---|---|
| CD34 | 1 | AGTTTTACCTGATTAACGAAATGCTCACACTTCTAAACTGAGGTCCTTAC |
| | | AGTAGATTCCTTTTGCAAGATTGTTACTGGCTTACAACTTAAAAATAAA |
| | | GGAAAATCACAAGGAAAGAAAAGTGGGGAAAAAATCGGAGGAAACTT |
| | | GCCCCTGCCCTGGCCACCGGCAAGGCTGCCACAAAGGGGTTAAAAGTTA |
| | | AGTGGAAGTGGAGCTTGAAGAAGTGGGATGGGGCCTCTCCAGAAAGCT |
| | | GAACGAGGCATCTGGAGCCCGAACAAACCTCCACCTTTTTTGGCCTCGA |
| | | CGGCGGCAACCCAGCCTCCCTCCTAACGCCCTCCGCCTTTGGGACCAAC |
| | | CAGGGGAGCTCAAGTTAGTAGCAGCCAAGGAGAGGCGCTGCCTTGCCA |
| | | AGACTAAAAAGGGAGGGGAGAAGAGAGGAAAAAAGCAAGAATCCCCC |
| | | ACCCCTCTCCCGGGCGGAGGGGGCGGGAAGAGCGCGTCCTGGCCAAGC |
| | | CGAGTAGTGTCTTCCACTCGGTGCGTCTCTCTAGGAGCCGCGCGGGAAG |
| | | GATGCTGGTCCGCAGGGGCGCGCGCGCAGGGCCCAGGATGCCGCGGGG |
| | | CTGGACCGCGCTTTGCTTG |
| CD34 | 2 | GGGAAATATGGAAGGTCACAGGAAAAGTTAACACAAGTTAGCAAAAAG |
| | | TTAACATAACACAAAAAGGTCTTGCAGGAAAAAAAAAAGAAAAGAAAA |
| | | GAAAGAAAAAGTCTCCAAGAATGGTTTGGACAGCCAAAATGAATACTT |
| | | ATAGTCACGTATACCTGCTCACTCCTGACGCTTCACTCACACACAGCAC |
| | | AGGATCTGGTGAGGCTATCACTAAATGTGCCACATTGTGGTTAAGTTTT |
| | | ACCTGATTAACGAAATGCTCACACTTCTAAACTGAGGTCCTTACAGTAG |
| | | ATTCCTTTTGCAAGATTGTTACTGGCTTACAACTTAAAAATAAAGGAAA |
| | | ATCACAAGGAAAGAAAAGTGGGGAAAAAATCGGAGGAAACTTGCCCCT |
| | | GCCCTGGCCACCGGCAAGGCTGCCACAAAGGGGTTAAAAGTTAAGTGG |
| | | AAGTGGAGCTTGAAGAAGTGGGATGGGGCCTCTCCAGAAAGCTGAACG |
| | | AGGCATCTGGAGCCCGAACAAACCTCCACCTTTTTTGGCCTCGACGGCG |
| | | GCAACCCAGCCTCCCTCCTAACGCCCTCCGCCTTTGGGACCAACCAGGG |
| | | GAGCTCAAGTTAGTAGCA |
| CD34 | 3 | AGACAACTGGGTTTAGAGAGGTGGAGACTGTTGATTGGTTCAGTGTGGC |
| | | ATTCAGACTACTTAGTTCAAATGCTGTTCAGAAAAACGGATTTTTCCAG |
| | | AGTTAGAACGTCTATCCAAGGACTTACTGGGAGACCTGCAGAATTGCTC |
| | | CTTTTCCTGAGGAATGAAGCAGCAGTGGCCTGAGAACTCATTTCTCTGT |
| | | AGCCTTGTTTCCTGGGGGTTTTTTGAGGCTCCAGTTTGGGCTCGTGTCTC |
| | | TGTGACCTGGAGTTTGGCTAACCACACTCTCCTGGCCTTATCCAAGCCCA |
| | | GTTGTTTTCCCTCAGCTGCTTCAAATTCCAGCTGGGTCCTGAGGCCAATC |
| | | TTGACCTTGCTTTGTGTAGGAGCAAAGGAGCCTGGGTTTTCCTGCCTTGG |
| | | GTCACAGCAGTGGGAAAATACCCAGGCTCCATTCCAACTGGGAGGACCC |
| | | TGTGGCCTTGTTGCAAGCAGCGGCCCTGCCCGCAAACAGGAAGCTTTCT |
| | | CCTCCACAGAGACCCAGTTCTGATGATGGTCACACACCCCAGCAGTTTT |
| | | CCCCTAACAGGAAAGTTGTCAGGGCTGTTCAGGCATTTCCTTCTCTGCCA |
| | | TCTGCCA |
| CD3 | | ACAGGTAGGCAGTATTGGACCCAGGATTCAAATCTCTGGCTGGGGTCTC |
| | | TAAAGCCCAACCTCCCACTGACAAGAAGCTGCTAGATCTGGTGTCCCTG |
| | | GCTGCCTAGTGAAGGGTCCTGAGAAAGATCAGCCTCCATGAGAAATCTA |
| | | GCTGCTACGGCTTGCGCTATGGGGCCGACGGCTTCTCTCAAGGGGCTTC |
| | | GAGATGTGGCAGTGTTTAGGTTGTGTGTAAATGTGGTTGCATTGTCAAT |
| | | AGGGACGCTAAAGTTCAGGCCACCTTTTCCATATTCTCTGCCAGCTCCCT |
| | | GCTCAGAGATAGAGCAATTTACACCGCTTCCTTCCTACCCTACCCCTAGC |
| | | CCACCCCCACTCTGAAAATTTCCCACCATCAACGGCAGAAAGCAGAGAA |
| | | GCAGACATCTTCTAGTTCCTCCCCCACTCTCCTCTTTCCGGTACCTGTGA |
| | | GTCAGCTAGGGGAGGGCAGCTCTCACCCAGGCTGATAGTTCGGTGACCT |
| | | GGCTTTATCTACTGGATGAGTTCCGCTGGGAGATGGAACATAGCACGTT |
| | | TCTCTCTGGCCTGGTACTGGCTACCCTTCTCTCGCAAGGTAAGGCTACTC |
| | | CAGGTGGG |
| CD4 | | TGGCCAGAGACGCCTAGAGGAACAGAGCCTGGTTAACAGTCACTCCTGG |
| | | TGTCTCAGATATTCTCTGCTCAGCCCACGCCCTCTCTTCCACACTGGGCC |
| | | ACCTATAAAGCCTCCACAGATACCCCTGGGGCACCCACTGGACACATGC |
| | | CCTCAGGGCCCCAGAGCAAGGAGCTGTTTGTGGGCTTACCACTGCTGTT |
| | | CCCATATGCCCCCAACTGCCTCCCACTTCTTTCCCCACAGCCTGGTCAGA |
| | | CATGGCGCTACCACTAATGGAATCTTTCTTGCCATCTTTTTCTTGCCGCTT |
| | | AACAGTGGCAGTGACAGTTTGACTCCTGATTTAAGCCTGATTCTGCTTAA |
| | | CTTTTTCCCTTGACTTTGGCATTTTCACTTTGACATGTTCCCTGAGAGCCT |
| | | GGGGGGTGGGGAACCCAGCTCCAGCTGGTGACGTTTGGGGCCGGCCCA |
| | | GGCCTAGGGTGTGGAGGAGCCTTGCCATCGGGCTTCCTGTCTCTCTTCAT |
| | | TTAAGCACGACTCTGCAGAAGGAACAAAGCACCCTCCCCACTGGGCTCC |
| | | TGGTTGCAGAGCTCCAAGTCCTCACACAGATACGCCTGTTTGAGAAGCA |
| | | GCGGG |
| CD8a | 1 | TCCTGGGGGAAGGGAGAGGGTCCTTCCTCGGTGAAAACTGGGGCTGCTC |
| | | TAGCGAGTTCCTCAGAAGCGGGCAGGTCGCTAGTTCCTCTTCCTTTTCAG |
| | | CCCTCAGTGCCCATTTTGCCAATAAAAAGTCCCAAGGTGACAGTACAAG |
| | | AGACGCCTTTAGTGAAGGCAAAGGAAGGGACACTCCCCTCCTTTGCTGC |
| | | CTACTCTCGCCCTCACTTCTTGAAATCTTTGGTCTCCCTTCACCCACTCTG |
| | | TCACTCTCACAAGACAACCATTTCCAAGGACTATTTCCAAGCCCTTTTCC |
| | | TCATCCCCAAACCCGCAGTTTTCAGCTGCCCCCAGTTGCCTGGCCAGGCT |
| | | GCCTCGACGGCCCTATTCACGGGCCCCAGCCTCCTCGCCGGGCTGGAAG |
| | | GCGACAACCGCGAAAAGGAGGGTGACTCTCCTCGGCGGGGGCTTCGGG |
| | | TGACATCACATCCTCCAAATGCGAAATCAGGCTCCGGGCCGGCCGAAGG |
| | | GCGCAACTTTCCCCCCTCGGCGCCCCACCGGCTCCCGCGCGCCTCCCCTC |
| | | GCGCCCGAGCTTCGAGCCAAGCAGCGTCCTGGGGAGCGCGTCATGGCCT |
| | | TACCAGT |
| CD8a | 2 | GTCAAAAAGGAAAGATGAGCCTGTAGTCCCAGCTACTCAGGGGGCTGG |
| | | GGTGGGAGGATCACTGGAGCTCAGGAGTCCCAAGGCCAGCCTGAGCAA |
| | | AACAGCGAGACTCCAGTCTTTTTTATTTTATTTTATTTTTTAAAGAAACA |
| | | AAAAGGAAGGGGACACACATGTGTTAGGGACAGAAAAGAGAAAACCG |
| | | CCTCTACCCAAGCATTCACCCACATCACCCACACCTCCCTGCAGAGCAC |
| | | CCAGAGCTGGGGGTGAAAGAAATGAGGTCCAAATGAGACAGCACAGGA |
| | | GCTGCCTCCAGGGCTTAAACAGACCAGCATTCCAGGCCGAGGGACCGCA |
| | | AGTGCAAGGGCGTGAAAGCACAGAGCGCAGGGGTTGAATGACTTCAAG |
| | | CCTGTGAAGCTGCAGCTGCAGGTGTATGGGAAAGGCAGGGCAGGGGGC |
| | | TGTGCGGAGGCTGGGAGGAGCCAGCACCCAAGGGCTGGTCAACCAAGC |
| | | TGGGGGTTGAATTTCCATCCAGCAATGCAGGCCATGGGAGGCTGCAGCA |
| | | GTGACGCTGTCAGATCCCCTTTGTGAGAATAATAATTTTTATAACAACGT |
| | | GGCTGGAGGACTGATCAG |
| CD8b | 1 | CAGTCCTTCGAAATTCTTAAGATCTAGGTCTTGCTGCACCCCCACAACCT |
| | | ACAAACAGCGTCGGGGCCTTCTCTGCACCTCCAGTTCCCAGCTCACCTCC |
| | | CTCAGTGTCACAGCCGGTTACCTTTCCTTCCTCCCTGGCAAGGGAGGGC |
| | | AAGACTTGGGGCTTGCTGACTCCAGGCCCAGCCCAGCCCGGGGCACCCA |
| | | GGAGCCCCTCAATTGCTACTCAAACAAGACAAGAAGCGGCCCGAGTTA |
| | | GTGGCCAGCTCCACCATGCACTACACATCCTGACCTCTCTGAGCCTCTAC |
| | | TGTCACTCGGGGTCACAACCCTTTCCTGAGCACCTCCCGGGGCAGGGGG |
| | | CGATGACACACATGCAGCTGCCTGGGGGAGGCCGGCGGTGTCCCCTCCT |
| | | TTCTGGAAAGCGGAGGGTCCTGGTGGGCTCTGGAAACGCAGCCCAGACC |
| | | TTTGCAATGCTAGGAGGATGAGGGCGGAGACCTCGCGGTCCCCAACACC |
| | | AGACTCCCGCCGCCACCGCGCCCGGTCCCGCCCTCCCCACTGCCCCCCC |
| | | AGCTCCCCGACCCAGGCGCCCCGCCCGGCCAGCTCCTCACCCACCCCAG |
| | | CCGCGACTGT |
| CD8b | 2 | TTTCCTTCCTCCCTGGCAAGGGAGGGCAAGACTTGGGGCTTGCTGACTC |
| | | CAGGCCCAGCCCAGCCCGGGGCACCCAGGAGCCCCTCAATTGCTACTCA |
| | | AACAAGACAAGAAGCGGCCCGAGTTAGTGGCCAGCTCCACCATGCACT |
| | | ACACATCCTGACCTCTCTGAGCCTCTACTGTCACTCGGGGTCACAACCCT |
| | | TTCCTGAGCACCTCCCGGGGCAGGGGGCGATGACACACATGCAGCTGCC |
| | | TGGGGGAGGCCGGCGGTGTCCCCTCCTTTCTGGAAAGCGGAGGGTCCTG |
| | | GTGGGCTCTGGAAACGCAGCCCAGACCTTTGCAATGCTAGGAGGATGAG |
| | | GGCGGAGACCTCGCGGTCCCCAACACCAGACTCCCGCCGCCACCGCGCC |
| | | CGGTCCCGCCCTCCCCACTGCCCCCCCAGCTCCCCGACCCAGGCGCCCC |
| | | GCCCGGCCAGCTCCTCACCCACCCCAGCCGCGACTGTCTCCGCCGAGCC |
| | | CCCGGGGCCAGGTGTCCCGGGCGCGCCACGATGCGGCCGCGGCTGTGGC |
| | | TCCTCTTGGCCGCGCAGCTGACAGGTAAGGCGGCGGCGCGCGGGCTACC |
| | | CAAGGGTCTGCG |

These 5 genes share high expression in CD4 and CD8 T cells:

| | |
|---|---|
| IRF4 | GCAACCTCCACCTCCAGTTCTCTTTGGACCATTCCTCCGTCTTCCGTTAC |
| | ACGCTCTGCAAAGCGAAGTCCCCTTCGCACCAGATTCCCGCTACTACAC |
| | GCCCCCCATTTCCCGCCCTGGCCACATCGCTGCAGTTTAGTGATTGACTG |
| | GCCTCCTGAGGTCCTGGCGCAAAGGCGAGATTCGCATTTCGCACCTCGC |
| | CCTTCGCGGGAAACGGCCCCAGTGACAGTCCCCGAAGCGGCGCGCGCCC |
| | GGCTGGAGGTGCGCTCTCCGGGCGCGGCGCGCGGAGGGTCGCCAAGGG |
| | CGCGGGAACCCCACCCCGGCCGCGGCAGCCCCCAGCCTTCACGCCGGCC |
| | CTGAGGCTCGCCCGCCCGGCCGGCCCCGGCTCTCGGCTTGCAAAGTCCC |
| | TCTCCCCAGTCCAACCCCCGGCCCCCACAGGCCTCGGCGCCCCGCCCCG |
| | CCCCAGGCCCCGCCCCAGAGAGTTCTATAAAGTTCCTCTTTCCCACCTCG |
| | CACTCTCAGTTTCACCGCTCGATCTTGGGACCCACCGCTGCCCTCAGCTC |
| | CGAGTCCAGGGCGAGGTAAGGGCTGGAGTCGGGCAGGAGGAGGGGTGT |
| | GAGGCTGATA |
| IFNG | TCTGATGAAGGACTTCCTCACCAAATTGTTCTTTTAACCGCATTCTTTCC |
| | TTGCTTTCTGGTCATTTGCAAGAAAAATTTTAAAAGGCTGCCCCTTTGTA |
| | AAGGTTTGAGAGGCCCTAGAATTTCGTTTTTCACTTGTTCCCAACCACAA |
| | GCAAATGATCAATGTGCTTTGTGAATGAAGAGTCAACATTTTACCAGGG |
| | CGAAGTGGGGAGGTACAAAAAAATTTCCAGTCCTTGAATGGTGTGAAGT |
| | AAAAGTGCCTTCAAAGAATCCCACCAGAATGGCACAGGTGGGCATAAT |
| | GGGTCTGTCTCATCGTCAAAGGACCCAAGGAGTCTAAAGGAAACTCTAA |
| | CTACAACACCCAAATGCCACAAAACCTTAGTTATTAATACAAACTATCA |
| | TCCCTGCCTATCTGTCACCATCTCATCTTAAAAAACTTGTGAAAATACGT |
| | AATCCTCAGGAGACTTCAATTAGGTATAAATACCAGCAGCCAGAGGAG |
| | GTGCAGCACATTGTTCTGATCATCTGAAGATCAGCTATTAGAAGAGAAA |
| | GATCAGTTAAGTCCTTTGGACCTGATCAGCTTGATACAAGAACTACTGA |
| | TTTCAACTTC |
| CSF2 | ATGTGAACTGTCAGTGGGGCAGGTCTGTGAGAGCTCCCCTCACACTCAA |
| | GTCTCTCACAGTGGCCAGAGAAGAGGAAGGCTGGAGTCAGAATGAGGC |
| | ACCAGGGCGGGCATAGCCTGCCCAAAGGCCCCTGGGATTACAGGCAGG |
| | ATGGGGAGCCCTATCTAAGTGTCTCCCACGCCCCACCCCAGCCATTCCA |
| | GGCCAGGAAGTCCAAACTGTGCCCCTCAGAGGGAGGGGGCAGCCTCAG |
| | GCCCATTCAGACTGCCCAGGGAGGGCTGGAGAGCCCTCAGGAAGGCGG |
| | GTGGGTGGGCTGTCGGTTCTTGGAAAGGTTCATTAATGAAAACCCCCAA |
| | GCCTGACCACCTAGGGAAAAGGCTCACCGTTCCCATGTGTGGCTGATAA |
| | GGGCCAGGAGATTCCACAGTTCAGGTAGTTCCCCCGCCTCCCTGGCATT |
| | TTGTGGTCACCATTAATCATTTCCTCTGTGTATTTAAGAGCTCTTTTGCCA |
| | GTGAGCCCAGTACACAGAGAGAAAGGCTAAAGTTCTCTGGAGGATGTG |
| | GCTGCAGAGCCTGCTGCTCTTGGGCACTGTGGCCTGCAGCATCTCTGCA |
| | CCCGCCCGCTCGCCC |
| IL2RA | TTTCAGGAGCCCAGGGCACTGTGGTGAAATGATGATGGCTAGTACAGGT |
| | TATAAGCCTTGGGGAATTATTTATGAATTCTCAGGATCCTTCAGTTCGCC |
| | GCATCCTTCTCCATTATTTGAATATTGGAGGCTGCCTGACCAGAATCTTG |
| | TCAGGACTTTGCTCCTTCATCCCAGGTGGTCCCGGCTGACTCCTGAGGAC |
| | GTTACAGCCCTGAGGGGAGGACTCAGCTTATGAAGTGCTGGGTGAGACC |
| | ACTGCCAAGAAGTGCTTGCTCACCCTACCTTCAACGGCAGGGGAATCTC |
| | CCTCTCCTTTTATGGGCGTAGCTGAAGAAAGGATTCATAAATGAAGTTC |
| | AATCCTTCTCATCAACCCCAGCCCACACCTCCAGCAATTGAACTTGAAA |
| | AAAAAAACCTGGTTTGAAAAATTACCGCAAACTATATTGTCATCAAAAA |
| | AAAAAAAAAAAAAAAACACTTCCTATATTTGAGATGAGAGAAGAGAGT |
| | GCTAGGCAGTTTCCTGGCTGAACACGCCAGCCCAATACTTAAAGAGAGC |
| | AACTCCTGACTCCGATAGAGACTGGATGGACCCACAAGGGTGACAGCCC |
| | AGGCGGACCG |
| ICOS | AATCTACAATGAATGCCACATAAATATCATTTCTCAGATTCCTATGATGC |
| | TCTTCTTTCAGATCTTTTCACTTCAATTTCTATAATAATTTTGTTTGTTTCT |
| | TGTCCTATTTCAAAGGCTTTCTTATCTCTGGAGCACCTAGCATAAGATAG |
| | AAATGTGTCAAAATATATGTTTTATTCATCATGTGAGTATTTTTAGGTCC |
| | TGTTAACCCCCATAACTATTGATTCAGAGAAGTAGGGTGGTTCTGAAAA |
| | ATACAGGCATAATCTCTTTAACTTGTTTTATAGGAACCAGAATAAGGGT |
| | AATGTTTTCCTCTGTCTTCAAAATCATCAATAATCCATGCATTGTTTAAC |
| | TCATGTCATAAGCAATAATGCCTTTCATATAGCCATTGGCATCAAAGAA |
| | GAAACACCCCCTTGATTTGATGGTAAGCGTGACACTACATAAACTCCCA |
| | GAAAACCCACTTCCTTTCCAGCAAATAGAAAACAACCGAGAGCCTGAAT |
| | TCACTGTCAGCTTTGAACACTGAACGCGAGGACTGTTAACTGTTTCTGGC |
| | AAACATGAAGTCAGGCCTCTGGTATTTCTTTCTCTTCTGCTTGCGCATTA |
| | AAG |

These 5 genes have higher expression in CD8 than CD4 T cells:

| | |
|---|---|
| XCL1 | AGCTCAGTGTGGCAGCAGCCTCTCTTCCCCTCCTGAGAGAGTCAAAGGG |
| | TGGCATCAGGGACTCATGATCCATGGTTGTGGAAGCCTCATGTCACACT |
| | GGATGTCACATGAGGTGGGATGGAACACAGTGACCACCCCACCTCATTT |
| | CCTTTACAGCTTCCGTGGTGGGCCATGGCAGTGAACAGCCTTCAGGCAT |
| | GTCTACGGTGGAAGATCTGAATTCAGGCTGGTGGCAGGAGACAACACA |
| | ACCACGTTTTCTTTTATGCATGCATTTGGTTTAATTGACACATTAACCAC |
| | AGACAAAGGGGTAAAGGCCACAAGGCGTTAGGTTAGTATGAACAGGGA |
| | AAGGGACTTTTTTTTTTTTTTTTTTTAAGAAAAATAAAAGCATCAGTATT |
| | GCAAAGACTTTCCATGATCCTACACCCACCTCGAAAGCCCCCTCTCACC |
| | ACAGGAAGTGCACTGACCACTGGAGGCATAAAAGAGGTCCTCAAAGAG |
| | CCCGATCCTCACTCTCCTTGCACAGCTCAGCAGGACCTCAGCCATGAGA |
| | CTTCTCATCCTGGCCCTCCTTGGCATCTGCTCTCTCACTGCATACATTGTG |
| | GAAGGTAAGTG |
| SLAM | GATGAAAAGACAGGCTACAGACCAAGAGAAAATATTTGTAAACCACAT |
| F7 | ATCTGACAAATGACTCTTATACTTGGAACATATAAGGAATTGTCAAAGC |
| | TCAACAGTAAAAAAAATAAAGAATCTGATTATAAAATGGACAAAAGAC |
| | ATAAATAGACATTTCACCAAGGAGGATATGGATATATAGATGGCAAATA |
| | AGCACATGAAAAGATGTTCAACATTATTAGGCTTTAGGGAAATGCAAAT |
| | TAAAGCCACAATGAGGTATCACTACAGCACCTATTAAAACAGCTAAAAT |
| | ATAAAATGGGAATATACCAAATGCTGATGAAGATGGGGAGCAAATAGA |
| | TCTCTCATAGATTGCTGGTGGCAAGGTAAAATGCTCTATTCACTCTGAAA |
| | ATAATTTAGCAATTACTCAATCTCACATGTCTGCGGCGTGACCCCTCCTG |
| | CTTCTTTAAATATCAGCTGGGGAAGAGGTCTGAGTAATACCTAAGAGGG |
| | AAGTGGCTTCATTTCAGTGGCTGACTTCCAGAGAGCAATATGGCTGGTTCC |
| | CCAACATGCCTCACCCTCATCTATATCCTTTGGCAGCTCACAGGTGAGTC |
| | CGGCCGGATT |
| IL4R | TTATTGAAGAATGTGCAACCACTCTCACTTGGAAGCCGGGCTGTTAGGA |
| | AGGGGAGGAGGATTCCAGTCGCCCAGCCCTCCCCCACCAAACGCAACTG |
| | CCCCGGCGCAAAAGAGGCCGCGGAGGCCAGGCAGGAGCAGGTCCTGGA |
| | GGCCTGGTCGGCGTGGGCGTTTTATTCCGAGACCAAGGGGATCCACTGC |
| | AGAGTTCTCCGCTGGGCGTGACCTCGGGCTACGGCGTGGGAGGAAGCGC |
| | GCGGCAAGACACCCAGCGAGGTGCTGGGGTCGCCCCCAGGAGAGGACG |
| | GCGGCTCGGACTGTCCGGCGGCGGCGGCGGGGACAGCGACAGGGGCGC |
| | GAGGTGGCCGGGACCCGGGCCGGGCGCGCCGGGCGGGGCGGCGCATGC |
| | AAATCTGCCGGGCGCCGGGGCGGGGAGCAGGAAGCCGGGGCGGGCTGG |
| | GTCTCCGCGCCCAGGAAAGCCCCGCGCGGCGCGGGCCAGGGAAGGGCC |
| | ACCCAGGGGTCCCCCACTTCCCGCTTGGGCGCCCGGACGGCGAATGGAG |
| | CAGGGGCGCGCAGGTAGGATCCGGGGCCCGCGCGCGGATCGGGTTGCG |
| | AAGGTATCGCCCGGGCACG |
| TNFS | GTCTCCCAGAAAGTCGTGGAAACGGATGCGGCCGACGGTGGTATTGGCC |
| F4 | TCAAAGTTGGGAGCCACGTCCCCGAGAAGCAGACCTCCGGGCATGGCG |
| | ACGGTGATGAGGGGGCGCCGCTGGGACAGCAAGCAACCGGTTGGTTCT |
| | GGCGAAGAAGCAGCCTGTCCCAGCGCGCGGAGGAATAAACGAAGGCGC |
| | GAGGGGCGGGGACTGGGGCGGCGGGGGCGGGGCCGCGGGAGGCCGGC |
| | CGCTGGGGGCCGGGCCGCGGGGGCTGGGCTGGGCGCGGGGCGGGGCGG |
| | GGCTGGGCGCGGGGCGGGGCTGGGCCGGGCCGGGCTGGGGCCGGGACG |
| | CGGCGCGAGCTGGACTACCGCGGTCGCTGTTGGTGGCGCCGCCGGGCCT |
| | GCGACTAGGTGGCATCCTTCAGACACTATAGGCCGTCTCTGCACACGTG |
| | ATGCGGGGCTCGGTCACGTTGCCCCCTGGAAGCTTGAGGATGCGCCAGG |
| | TTTGACTCTGCAGGGCGTACGCGCTTTAGGGATGGAAGGAAGAGGAAC |
| | GCGGCAGGAAGGCGAGCCCCAAGGTGGAGAATCGCCTGGGCGCGCAGG |
| | CTGCGGCGGCTTCGCACAGCC |
| CD72 | GCCCACCCCCCTACAGACCCCACAAATGCCCCTGGGTCCCTGGACCTCT |
| | GAGGACCCCACCCTGGGCCTCCCCAGGAGAGGCCCAGGTCGCGGTTAA |
| | GGGCAGGTAGCTGGGGATGCGGAGGAGGGAGGGAGGGAGGCTTCGGG |
| | GGCGGACACCGGATGCGGGGAACCACCGGCAGCGGGATGTGGGGTTCT |
| | GAGGGCTGCGGTGCTTCTGAAGATGGCTCAGCGTCGCGCCAGGTGGACG |
| | TGAGAGCTTTACCCTGGAGGAGGCGGGGGTTGGAGTCCCGCCTACCCAC |
| | TGGGACAAGCCAAGGGGTCAAACGCCCCCAACCCAGCCCGCAGATCTC |
| | CTCGAAGCACCCGGTTCTCCTGGCCCGCCCAGACCCACGGCGCTCGCCG |
| | CCTTCGCCCGCTTAGGACTGAGTCCGCAGCGCCGCCGCCTGGCGAGGGG |
| | CGGAGTTGCCACCACTTCTGCGCAGGCGGGATGCAGCCTGGCCCGCGGC |
| | ATCCCGGGAGTTGTAGTCTCGACGCTTCGGGGCCACCCCAGGGTCTGGT |
| | CCCTGACGACGCGCAGTGAGGGCCCCGCCGCTACCCCAGCAGTCGCCTC |
| | CCAAGTTCGCGGAACGC |

Lung Cancer Markers

| | Protein (ligand, secreted protein and/or receptor | Tissue |
|---|---|---|
| Gene | and/or structural homologue) | Specificity |
| STATH | Statherin | 1719 |
| | MKFLVFAFILALMVSMIGA, | |
| | DSepSepEEKFLRRIGRFG, or | |
| | MKFLVFAFILALMVSMIGADSepSepEEKFLRRIG | |
| | RFG (Sep = phosphoserine)) | |
| | FIG. 18B depicts the first 62 amino acids of | |
| | statherin, whereby either the signal peptide sequence | |
| | MKFLVFAFILALMVSMIGA or a longer sequence | |
| | containing DSepSepEEKFLRRIGRFG (Sep = | |
| | phosphoserine) may be used to confer enhanced lung | |
| | “secretomimetic” behavior of nanoparticles. In | |
| | addition to targeting ligands being utilized that | |
| | correspond to surface markers on a target cell type, | |
| | secreted proteins may also be used to enhance | |
| | nanoparticle properties in a given specific | |
| | microenvironment. | |
| SFTPB | Surfactant protein B | 912 |
| | CWLCRALIKRIQAMIPKGGRMLPQLVCRLVLRCS | |
| | FIG. 18C depicts Surfactant Protein B (see | |
| | Nicholas Rego and David Koes 3Dmol.js: molecular | |
| | visualization with WebGL Bioinformatics (2015) 31 | |
| | (8): 1322-1324 doi:10.1093/bioinformatics/btu829). | |
| | Its sequence corresponds to | |
| | CWLCRALIKRIQAMIPKGGRMLPQLVCRLVLRC | |
| | S and this protein is found upregulated in lung cancer | |
| | as a marker with an organ specificity index of 912. | |
| | NMR structures of this protein can be found at | |
| | http://www.bmrb.wisc.edu/dictionary/starviewer/?ent | |
| | ry=20028. | |
| CALCA | Calcitonin related polypeptide alpha | 78 |
| | FIG. 18D depicts a crystal structure of Calcitonin | |
| | related polypeptide alpha (PDB ID 2JXZ.A). | |
| BPIFB2 | BPI fold containing family B member 2 | 23 |
| | MAWASRLGLLLALLLPVVGA (BPI fold | |
| | containing family B member 2 signal peptide) | |
| | FIG. 18E depicts a structural homologue of BPI | |
| | fold containing family B member 2: BPI fold | |
| | containing family B member 1. Due to the sequence | |
| | similarity, and despite the absence of a crystal | |
| | structure for BPI fold containing family B member 2, | |
| | it is possible to predict ideal sequences for extracting | |
| | ligand-receptor or secreted protein - environment | |
| | interactions. (PDB ID 4KGH) | |
| | BPI fold containing family B member 2 contains a | |
| | predicted signal peptide sequence (1-20) along with | |
| | the rest of its mature chain (21-458). Proteins with | |
| | signal peptide domains are highly predictable (Zhang | |
| | Z., Henzel W. J. Protein Sci. 13: 2819-2824(2004)) | |
| | and these short sequences can be used to mimic a | |
| | given “secretome” environment as a nanoparticle | |
| | “stealth domain. | |
| NAPSA | Napsin A aspartic peptidase | 14 |
| | (1)MSPPPLLQPLLLLLPLLNVEPSGA(25)TLIRIPL | |
| | HRVQPGRRILNLLRGWREPAELPKLGAPSPGDK | |
| | PIFVPLSNYRDGYTTDLIPKPLAPSRPMGPSLPF | |
| | NMELGG (Napsin A 1-104) | |
| | and/or | |
| | CWLCRALIKRIQAMIPKGGRMLPQLVCRLVLRC | |
| | S (Surfactant protein B) | |
| | FIG. 18F depicts lung adenocarcinoma and renal | |
| | cell carcinoma relative expression of Napsin A | |
| | aspartic peptidase (Mol Cell Proteomics. 2014 | |
| | February; 13(2): 397-406. | |
| | doi:10.1074/mcp.M113.035600. | |
| | Epub 2013 Dec. 5). Napsin A aspartic peptidase | |
| | interacts proteolytically with Napsin-A, which | |
| | presents Napsin-A as an ideal nanoparticle constituent | |
| | for Napsin A aspartic peptidase processing in lung | |
| | and kidney cancers overexpressing this protease. | |
| | Either the signal peptide (1-24), entire chain (1-104), | |
| | or specific sequences that are cleaved as determined | |
| | by mass spectroscopy of Napsin-A in the presence of | |
| | Napsin A aspartic peptidase may be utilized. | |
| | Similarly, Napsin A aspartic peptidase overexpression | |
| | may be used along with surfactant protein B surface | |
| | coatings on nanoparticles due to Napsin A aspartic | |
| | peptidase's proteolytic effect on Surfactant protein B. | |
| | “This gene encodes a member of the peptidase A1 | |
| | family of aspartic proteases. The encoded | |
| | preproprotein is proteolytically processed to generate | |
| | an activation peptide and the mature protease. The | |
| | activation peptides of aspartic proteinases function as | |
| | inhibitors of the protease active site. These peptide | |
| | segments, or pro-parts, are deemed important for | |
| | correct folding, targeting, and control of the | |
| | activation of aspartic proteinase zymogens. The | |
| | encoded protease may play a role in the proteolytic | |
| | processing of pulmonary surfactant protein B in the | |
| | lung and may function in protein catabolism in the | |
| | renal proximal tubules. This gene has been described | |
| | as a marker for lung adenocarcinoma and renal cell | |
| | carcinoma.” [provided by RefSeq, February 2016] | |
| | (https://www.ncbi.nlm.nih.gov/gene/9476) | |
| PENK | Proenkephalin | 9 |
| SCGB1A1 | Secretoglobin family 1A member 1 | 9 |
| BPIFA1 | BPI fold containing family A member 1 | 8 |
| NR0B1 | Nuclear receptor subfamily 0 group B member 1 | 8 |
| | FIG. 18G depicts crystal structures of a potential | |
| | binding partner (top, COPS2: PDB IDs 4D10, 4D18, | |
| | 4WSN) to nuclear receptor subfamily 0 group B | |
| | member 1 (bottom, PDB ID 4RWV) for programming | |
| | subcellular-specific behavior of a nuclear receptor | |
| | (Nuclear receptor subfamily 0 group B member 1) | |
| | that is overexpressed on the target cell/tissue/organ. | |
| | This protein exhibits shuttling between the cytosol | |
| | and the nucleus, therefore inclusion of ligands | |
| | interacting with this protein may facilitate nuclear | |
| | transport and nuclear-specific release. This protein is | |
| | known to have protein-protein interactions with other | |
| | nuclear receptors and transcription factors, including | |
| | NR5A1, NR5A2, NR0B2 and COPS2 (Suzuki T., | |
| | Kasahara M., Yoshioka H., Morohashi K., Umesono | |
| | K. Mol. Cell. Biol. 23: 238-249(2003)). Therefore, | |
| | binding domains or transcription factor sequences | |
| | may be incorporated along with an electrostatic core | |
| | of nanoparticles to generate cell/tissue/organ/cancer- | |
| | specific subcellular trafficking (e.g. in place or in | |
| | conjunction with H2A or H2B histone fragments, or | |
| | as a sequence within an otherwise electrostatic | |
| | sequence). | |
| PON3 | Paraoxonase 3 | 8 |
| | FIG. 18H depicts how paroxonase 3 (left, PDB ID | |
| | 1v04) overexpression may be used to engineer | |
| | polymer chains (right) modified with cleavable N- | |
| | acyl homoserine lactone in order to encourage | |
| | substrate specificity through degradation in a tissue- | |
| | enriched way. Various other substrates with specific | |
| | cleavage activity may be used. | |
| KRT31 | Keratin, type I cuticular Hal | 6 |
| | FIG. 18I depicts structural homologues of Keratin, | |
| | type I cuticular Ha1. Left: keratin 5 and 14 (PDB ID | |
| | 3tnu). Top right: keratin type I cytoskeletal 14 (PDB | |
| | ID 3TNU.A). Bottom right: keratin type II | |
| | cytoskeletal 5 (PDB ID 3TNU.B). Keratin sequences | |
| | may be utilized to mimic local environmental ECM | |
| | upon the nanoparticle surface, facilitating interaction | |
| | with intermediate filaments upregulated in the target | |
| | tissue, and allowing for enhanced nanoparticle | |
| | binding to complementary keratin-binding cells. | |
| | Additionally, cysteine-rich keratin sequences may be | |
| | utilized as cross-linking sequences for nanoparticle | |
| | cores or surfaces, as well as possessing affinity for | |
| | intermediate filaments in various tissues. | |
| | FIGS. 18J1-3 depicts high homology of coils 1A, | |
| | 1B, and 2 between keratin, type I cuticular Ha1 (top) | |
| | and keratin, type I cytoskeletal 14 (bottom). | |
| SCGB3A1 | Secretoglobin family 3A member 1 | 6 |
| DSG3 | Desmoglein 3 | 5 |
| SERPINB12 | Serpin family B member 12 | 5 |
| KIT | CD117 | 0.62 |
| | FIGS. 18O-18Q depict how to use sequence | |
| | alignment techniques to determine optimal domains | |
| | for creating a targeting ligand specific to CD117/c- | |
| | Kit. | |
| | Also known as mast/stem cell growth factor receptor | |
| | (SCFR), or c-Kit, CD117 serves as a unique marker | |
| | for long-term hematopoietic stem cells (ltHSC) and | |
| | additional cells of the hematopoietic lineage. | |
| CD44 | CD44 | 0.57 |
| | FIG. 18N depicts a crystal structure of the | |
| | hyaluronan binding domain of human CD44 (PDB ID | |
| | 1UUH) and a corresponding structure of hyaluronan/ | |
| | hyaluronic acid, which can readily be included upon | |
| | nanoparticle surfaces or as an anionic core | |
| | nanoparticle component, and may serve as a CD44- | |
| | specific targeting ligand. | |
| ALCAM | CD166 | 0.5 |
| | FIG. 18O depicts the region of CD166(28-120) | |
| | which mediates CD6 binding via its N-terminal Ig- | |
| | like V Type 1 domain. A signaling peptide sequence | |
| | (1-25) may also be utilized individually or as (1-120). | |
| | FIG. 18T depicts how CD166 mediates CD6 | |
| | binding via its N-terminal Ig-like V Type 1 domain | |
| | (square highlighted on left). The membrane-proximal | |
| | CD6 SRCR domain (labeled Sc) mediates binding to | |
| | the N-terminal Ig-like V Type 1 domain of CD166 | |
| | (middle, PMID: 26146185). A small domain | |
| | signature is identified on the C-terminus of human | |
| | CD6, whereby amino acids D291-N353 (62AA) | |
| | dictate binding to CD166 (top right, PMID: | |
| | 26146185). Correspondingly, a small domain | |
| | signature is identified on the N-terminus of human | |
| | CD166, whereby amino acids F53-E118 (65AA) | |
| | dictate binding to CD6. | |
| | FIG. 18Q depicts two techniques for forming de | |
| | novo CD6-specific ligands, whereby a triple-domain | |
| | electrostatic affinity sequence matches dimensions of | |
| | the binding pocket of CD6. Dimensional reduction | |
| | techniques of a 2-dimensional electrostatic pocket | |
| | allow for creation of short peptide sequences with | |
| | corresponding electrostatic affinity for the t-shaped | |
| | domain. | |
| | Conversely, CD166 fragments may be used to target | |
| | CD6, which is a T cell marker and signals for T cell | |
| | activation upon binding to CD166 (typically | |
| | expressed on endothelial cells). The use of this ligand | |
| | and its concomitant receptor is not only restricted to | |
| | lung cancer, but may also be utilized for targeting | |
| | various endothelial cell and immune cell populations | |
| | as part of a nanoparticle coating bearing one or more | |
| | targeting ligands. Truncated fragments exhibiting | |
| | only partial electrostatic complementarity may be | |
| | utilized in these embodiments as well. For example, | |
| | while CD166(53-118) dictates primary binding to | |
| | CD6 and has notable t-shaped electrostatic structure | |
| | (where the horizontal axis of the t represent anionic | |
| | pockets, and the vertical axis represents cationic | |
| | pockets | |
| | De novo CD6-targeting sequences with variable | |
| | specificity vs. selectivity may include: | |
| | ERE | |
| | RRRRR | |
| | RRRRRR | |
| | RRRRRRR | |
| | EEREE | |
| | EEKRKEE | |
| | EGGRRGGE | |
| | EEGGRRGGEE | |
| | EERRCRREE | |
| | EERCREE | |
| | ERCRE | |
| | CERE | |
| | CEERREE | |
| | EREC | |
| | EERREEC | |
| | CD6-targeting sequences may also include the | |
| | following compositions, which allow for anchoring to | |
| | various linker-anchor domains and cysteine-binding | |
| | substrates: | |
| | RKCRCKR | |
| | CRRRRRR | |
| | CCRRRRRR | |
| | CCCRRRRRR | |
| | RRRRRRC | |
| | RRRRRRCC | |
| | RRRRRRCCC | |
| | De novo CD166-targeting sequences with variable | |
| | specificity vs. selectivity may include: | |
| | RER | |
| | EEEEE | |
| | EEEEEE | |
| | EEEEEEE | |
| | RRERR | |
| | RKEEEKR | |
| | RGGEEGGR | |
| | RRGGEEGGRR | |
| | RREECEERR | |
| | RRECERR | |
| | RECER | |
| | CRER | |
| | CRREERR | |
| | RERC | |
| | RREERRC | |
| | In this example, sequential locations of D291-E293 | |
| | can be modeled to understand approximate required | |
| | AA length of a complementary binding substrate | |
| | (left). Due to the large t-shaped electrostatic binding | |
| | pocket (middle), complementary electrostatic peptide | |
| | sequences may be assembled. | |
| | These peptides include one or more “staple” domains | |
| | (e.g. a cationic domain, anionic domain, and cationic | |
| | domain) (“oppositely charged t-complementary | |
| | domain”/“staple domain”) and each domain is | |
| | between 2-7AA. Some domains may be 1-3AA. In | |
| | other embodiments, 7-15, 7-30, 15-30, 20-25, 20-30, | |
| | and similarly sized electrostatic domains may be | |
| | utilized to enable homogenous charge pocket | |
| | complementary (e.g. complete neutralization or | |
| | switching of electrostatic potential) for charged | |
| | pockets on zwitterionic surfaces <10 nm. | |
| | Truncated fragments exhibiting complete electrostatic | |
| | complementarity in a two-dimensional approximate | |
| | structure may be utilized in embodiments where a | |
| | target receptor or protein must bind to a given peptide | |
| | sequence. Previously, we detailed this approach in | |
| | condensing Cas9 RNPs with PLR10 (~3.5 Å) (FIG. | |
| | 19Y), which matches anionic binding pocket sizes on | |
| | the overall protein (12 nm). In this example, | |
| | CD166(53-118) dictates primary binding to CD6 and | |
| | both proteins have notable t-shaped electrostatic | |
| | structures (where the horizontal axis of the t represent | |
| | either anionic or cationic pockets, and the vertical | |
| | axis represents either cationic or anionic pockets). | |
| | This electrostatic structure may be exploited with | |
| | triple-charge-domain polypeptide or polymer | |
| | sequences matching the binding pocket's length | |
| | (approximately 3-6AA). | |
| | “Cell adhesion molecule that mediates both | |
| | heterotypic cell-cell contacts via its interaction with | |
| | CD6, as well as homotypic cell-cell contacts | |
| | (PubMed: 7760007, PubMed: 15496415, | |
| | PubMed: 15048703, PubMed: 16352806, | |
| | PubMed: 23169771, PubMed: 24945728). Promotes T- | |
| | cell activation and proliferation via its interactions | |
| | with CD6 (PubMed: 15048703, PubMed: 16352806, | |
| | PubMed: 24945728). Contributes to the formation and | |
| | maturation of the immunological synapse via its | |
| | interactions with CD6 (PubMed: 15294938, | |
| | PubMed: 16352806). Mediates homotypic interactions | |
| | with cells that express ALCAM (PubMed: 15496415, | |
| | PubMed: 16352806). Required for normal | |
| | hematopoietic stem cell engraftment in the bone | |
| | marrow (PubMed: 24740813). Mediates attachment of | |
| | dendritic cells onto endothelial cells via homotypic | |
| | interaction (PubMed: 23169771). Inhibits endothelial | |
| | cell migration and promotes endothelial tube | |
| | formation via homotypic interactions | |
| | (PubMed: 15496415, PubMed: 23169771). Required | |
| | for normal organization of the lymph vessel network. | |
| | Required for normal hematopoietic stem cell | |
| | engraftment in the bone marrow. Plays a role in | |
| | hematopoiesis; required for normal numbers of | |
| | hematopoietic stem cells in bone marrow. Promotes | |
| | in vitro osteoblast proliferation and differentiation | |
| | (By similarity). Promotes neurite extension, axon | |
| | growth and axon guidance; axons grow preferentially | |
| | on surfaces that contain ALCAM. Mediates | |
| | outgrowth and pathfinding for retinal ganglion cell | |
| | axons” | |
| | http://www.rcsb.org/pdb/protein/Q13740 | |
| PROM1 | CD133 | 0 |
| | FIG. 18R depicts ScFV critical sequences for | |
| | CD133 (prominin-1) binding (Xia, Jing, et al. | |
| | “Isolation, identification and expression of specific | |
| | human CD133 antibodies.” Scientific reports 3 | |
| | (2013): 3320). | |
| | LQNAPRS is known to bind to mouse CD133 | |
| | (PMID: 22228571) | |

Table 6 illustrates several unique ligand derivation approaches for overexpressed markers and secreted proteins in a lung cancer dataset (GTEx Portal).

Breast Cancer Markers

| | | Tissue |
|---|---|---|
| Gene | Protein (ligand and/or receptor) | Specificity |
| PIP | Prolactin induced protein | 618 |
| | Prolactin-induced protein interacts with Zinc-alpha-2- | |
| | glycoprotein (ZAG) (PDB ID 3es6) via two domains, α1 and α3 | |
| | (FIG. 18S). | |
| | FIG. 18S depicts hydrogen bonding residues involved in PIP | |
| | binding to α1, α2 and α3 domains of Zinc-alpha-2-glycoprotein | |
| | (ZAG) (PDB ID 3es6). | |
| | Prolactin-induced protein interacts with Zinc-alpha-2- | |
| | glycoprotein (ZAG) (PDB ID 3es6) via E229-G238 in the α3 | |
| | domain, and D23, D45 and Q28 (which are less than 5AA apart if | |
| | a charge-based triangulation approach for de novo ligand | |
| | domains is utilized (as in FIG. 18M). The interactions between | |
| | D23, Q28 and D45 on the α1 domain of ZAG with T79, S47 and | |
| | R72 on PIP can be reproduced by creating cyclical peptide | |
| | sequences displaying the appropriate amino acids (D, D, Q) at the | |
| | with sufficient spacing to allow for reproduction of native | |
| | hydrogen bonding. Larger sequences (e.g. D23-D45 for α1 | |
| | domain) may also be utilized. Correspondingly, E229-G238 | |
| | from the α3 domain (a mere 10 amino acids) can be used to | |
| | confer binding to G52, T59, T60 and K68 on PIP. | |
| | Additional cysteine or selenocysteine substitutions at glycine | |
| | residues with SH/SeH protection groups may be used to allow for | |
| | initial “ring-forming” C- and N-terminal cysteine cross-linking | |
| | before deprotection and subsequent attachment to an anchor or | |
| | anchor-linker pairing as described elsewhere. Other linker | |
| | domain sequences, PEG, and the like may be utilized in place of | |
| | GGS/GGGS sequences to create the appropriate spacing | |
| | structures. | |
| | ZAG(1-298): | |
| | MVRMVPVLLSLLLLLGPAVPQENQDGRYSLTYIYTGLSKH | |
| | VEDVPAFQALGSLNDLQFFRYNSKDRKSQPMGLWRQVEG | |
| | MEDWKQDSQLQKAREDIFMETLKDIVEYYNDSNGSHVLQ | |
| | GFGCEIENNRSSGAFWKYYYDGKDYIEFNKEIPAWVPFDP | |
| | AAQITKQKWEAEPVYVQRAKAYLEEECPATLRKYLKYSK | |
| | NILDRQDPPSVVVTSHQAPGEKKKLKCLAYDFYPGKIDVH | |
| | WTRAGEVQEPELRGDVLHNGNGTYQSWVVVAVPPQDTA | |
| | PYSCHVQHSSLAQPLVVPWEAS | |
| | ZAG Signal Peptide (1-20): | |
| | MVRMVPVLLSLLLLLGPAVP | |
| | ZAG-derived (α1) PIP-targeting sequences (D23-D45): | |
| | DVPAFQALGSLNDLQFFRYNSKD (anchor domain or anchor- | |
| | linker domain should be conjugated to amino acids 30-40 in order | |
| | to facilitate appropriate presentation of the critical D23, D45, and | |
| | Q28 domains to T79, R72, and S47 domains, respectively, on | |
| | PIP. | |
| | ZAG-derived (α3) PIP-targeting sequence (D229-G238): | |
| | ELRGDVLHNG (anchor domain or anchor-linker domain should | |
| | be at N-terminal of this sequence) | |
| | De novo ZAG-derived (α1) cyclical PIP-targeting sequences: | |
| | CGSDGGGSDGGGSQGC | |
| | CSDGGSDGGSQGC | |
| | Prolactin induced protein chelating amino acid sequence: | |
| | DVPAFQALGSLNDLQFFRYNSKD, with one or more cysteine | |
| | substitutions, may be bound to a ELRGDVLHNG-PEG-SH or | |
| | ELRGDVLHNG-spacer-SH in order to create a single peptide | |
| | with inactivation potential due to chelation of PIP. This may be | |
| | utilized to regulate cell invasion and integrin signaling in | |
| | estrogen receptor negative breast cancers. | |
| | (https://doi.org/10.1186/bcr3232) | |
| | ZAG may be forcibly expressed in its full form in an immune cell | |
| | population in order to confer greater affinity for PIP, and | |
| | subsequent chemotaxis of immune cell populations towards PIP- | |
| | expressing cells. | |
| | Alternatively, siRNA for PIP may be delivered to lung cells to | |
| | reduce the effect of PIP on the proliferation of certain breast | |
| | cancers. | |
| | ZAG shows a high degree of sequence homology to MHC-I, | |
| | where similar modeling approaches may he applied. | |
| | “Figure depicts overall structure of the ZAG-PIP complex. The | |
| | α1 domain of ZAG is shown in cyan, the α2 domain in green, and | |
| | the α3 domain in red; PIP is indicated in blue. The secondary- | |
| | structure elements are given in the corresponding color, α1 | |
| | domain: β1 (Arg7-Leu17), β2 (Phe27-Leu33), β3 (Leu36- | |
| | Asn42), H2 (Lys64-Tyr87). α2 domain: β1 (Val96-Glu106), β2 | |
| | (Arg109-Tyr119), β3 (Lys122-Asn129), β4 (Ala133-Pro136), | |
| | H1′ (Ala141-Trp148), and H2′ (Val155-Leu181). α3 domain: β1 | |
| | (Ser188-His194), β2 (Lys201-Phe210), β3 (Ile215-Arg221), β4 | |
| | (Glu229-His236), β5 (Thr241-Val250), β6 (Tyr258-Gln263), | |
| | and β7 (Leu271-Pro274). PIP: v1 (Ile9-Val18), β2 (Val24- | |
| | Thr32), β3 (Met38-Ser46), β4 (Tyr56-Leu62), β5 (Pro67- | |
| | Phe74), β6 (Val82-Val88), and β7 (Arg108-Val117).” | |
| | Hassan, M. I., Bilgrami, S., Kumar, V., Singh, N., Yadav, S., | |
| | Kaur, P., & Singh, T. P. (2008). Crystal Structure of the Novel | |
| | Complex Formed between Zinc α2-Glycoprotein (ZAG) and | |
| | Prolactin-Inducible Protein (PIP) from Human Seminal Plasma. | |
| | Journal of Molecular Biology, 384(3), 663-672. | |
| | doi:10.1016/j.jmb.2008.09.072 | |
| CST5 | Cystatin D | 363 |
| DCD | Dermcidin | 96 |
| LACRT | Lacritin | 6 |
| TFAP2B | Transcription factor AP-2 beta | 0 |

Table 7 illustrates a unique ligand derivation approache for the most overexpressed markers and secreted proteins in a breast cancer dataset (GTEx Portal).

Glioma Markers

| | | Tissue |
|---|---|---|
| Gene | Protein (ligand and/or receptor) | Specificity |
| TMEM235 | Transmembrane protein 235 | 100 |
| MMD2 | Monocyte to macrophage | 83 |
| | differentiation associated 2 | |
| GPR37L1 | G protein-coupled receptor 37 like 1 | 65 |
| GPM6A | Glycoprotein M6A | 36 |
| TMEM59L | Transmembrane protein 59 like | 16 |
| CADM2 | Cell adhesion molecule 2 | 14 |
| DSCAM | DS Cell adhesion molecule | 11 |

Table 8 illustrates several overexpressed markers in a glioma cancer dataset (GTEx Portal).

The identified proteins above may represent ligand and/or receptor and/or structural homologues of concomitant ligand/receptor/secretome profiles of target cell populations. In other words, a target cell/tissue/organ will contain a certain set of overexpressed genes. In the above examples, several cancer-enriched markers are shown for a variety of cancer markers based on transcriptomics and/or proteomics data from the Human Protein Atlas, as compared to healthy tissues/organs through selection algorithms detailed throughout this application. In the above examples, crystal structures represent a ligand OR a receptor OR a secreted protein for a given receptor profile or secreted microenvironment of a cell/tissue/organ. Ligands may represent locally secreted (e.g. lung-cancer-enriched) proteins and protein fragments thereof, in order to take part in an autocrine and/or paracrine signaling environment that is cell, tissue, organ, and/or cancer enriched, or to mimic physicochemical properties that are ideal for that environment (e.g. Surfactant protein B being a mucoadsorptive molecule, as shown in FIG. 18C).

In an illustrative example of keratin 31 (FIGS. 18I and 18J, which is overexpressed in a representative lung cancer dataset, full structural modeling data is not available (e.g. crystal structure or NMR data). However, abundant data is available on other forms of keratin. Using sequence alignment techniques and assessment of various conserved domains, it is possible to predict Keratin 31's alpha helical structure and therefore either utilize keratin 31 fragments as ligands for local tumor microenvironments (with the assumption that the secreted protein will interact with ECM components and receptors in the local environment), or alternatively create targeting ligands for keratin 31. Various hydrophobic domains, hydrophilic domains, alpha helical domains, beta sheet domains, and random coil domains may be compared, selectively mutated, and synthesized. In many cases, proteins may have large regions where ligand binding is not necessary to model (e.g. structural protein components that are not part of the protein-protein interaction between a protein and its receptor or ligand). For example, only 5%, 10% or 20% of a larger protein may be relevant for creating a targeting ligand or identifying a binding site in a receptor. In many examples, fewer than 7 amino acids are necessary to create a targeting ligand. In other examples, 7-30 amino acids are frequently used. 30-80 or 80-200 amino acids may be used in other examples.

Domains of 30-80 amino acids may also be ligated together (e.g. through native chemical ligation) in order to assemble larger proteins that typically can only be synthesized recombinantly. This offers the advantage of controlling protein folding in stages and sequentially assembling proteins with appropriate tertiary and quaternary structures. Such techniques of peptide synthesis may also be utilized for assembling protein components of gene editing materials such as TALENs, whereby 31-33 amino acid RVD (repeat variable diresidue) sequences may be synthesized and subsequently “daisy chained” together through native chemical ligation (FIG. 20B) rather than DNA-based assembly techniques (e.g. Golden Gate TALENs or open assembly techniques utilizing DNA ligation, such as depicted in FIG. 20A). Similar techniques for protein assembly can be imagined for CRISPR proteins, meganucleases, megaTALs, recombinases, and other genome-editing proteins detailed further within this disclosure. In other embodiments, these “polypeptide block assemblies” may create secreted/immunomodulatory proteins or any other protein classes that are typically limited to recombinant means of synthesis.

### Various Domains May be Compared Between Two Similar Proteins in Order to Establish Conserved Patterns.

### Exemplary Sequence Alignment

In the following examples (FIGS. 18O-18Q), mouse SCF (kit ligand) is aligned to human SCF (kit ligand) in order to determine predicted key sequences for a ligand. Despite significant differences in the structures of the two proteins, the signaling domains are highly aligned. This approach may be used to derive targeting ligands when there is an absence of structural data, when a higher degree of clinical translatability between different animal models (e.g. mouse to human) is desired, and/or to create broad classes of peptide targeting ligands for a given receptor class with high sequence homology.

In this illustrative example, sequences from one protein align highly with the signaling domain of another protein. Even in the absence of structural data on the entire protein, the relevant portion for designing a peptide targeting ligand can be predicted and modeled with high precision and accuracy across various protein classes. The need for large tertiary structures to align is eliminated when binding motifs between peptide ligands and their cognate receptors represent small portions of the overall protein. In some cases, techniques such as those described in: AlQuraishi M, Cell Syst. 2019 Apr. 24; 8(4):292-301. Epub 2019 Apr. 17; can be used (e.g., in some cases when the designed candidate protein 20 or more amino acids in length). Such techniques can be used to compare the structure of larger sequences when structural data is limited or not available prior to extracting and optimizing smaller binding sequences

In the following protein sequence alignment script (EMBOSS Needle), human and mouse SCF isoform 1 are found to have 89.7% sequence similarity (FIG. 18M). However, their structures are nearly identically aligned. Therefore, a high degree of permissivity is anticipated in deriving finite sequences from each variant to facilitate targeting the given receptor (mouse or human c-Kit). This approach is broadly applicable to sets of receptors with cognate ligands, or for secreted proteins (including signal peptides) with cognate receptors or desired activity in a target cell/tissue/organ.

| | | Enriched/ | | | |
|---|---|---|---|---|---|
| | Role in | upregulated | | Endogenous | |
| Proteolytic | pathological | in diseased | | Enzyme | |
| enzymes | conditions | state | MMP substrates | Inhibitors | References |
| MMP2 | Rheumatoid | Over-active | PLG~LYL, | | {1} |
| | Arthritis | | GPLG~IAGQ, | | |
| | | | GPLG~VRGK, | | |
| | | | HPVG~LLAR | | |
| | | | (MMP2); {1} | | |
| MMP1 and | Inflammation | Over-active | Type I Collagen | | {1} {3} |
| MMP7 | | | (MMP1); Fas | | |
| | | | ligand, Fibronectin | | |
| | | | (MMP7) {3} | | |
| MMP1, 2, 3, 7, | Colorectal | Over- | PLG~LYL. | | {1}, {4} |
| 9, 13 and MT1- | cancers | expressed | GPLG~IAGQ | | |
| MMP | | | (MMP2); {1} | | |
| (MMP14) | | | PLG~LYAL, ala- | | |
| | | | AALG~NVA-P | | |
| | | | (MMP9) {1} | | |
| MT1-MMP | Angiogenesis | Increased | Type I Collagen, | | {1} {3} |
| (MMP14), | | levels | Cell surface tissue | | |
| MMP2 and | | | transglutaminase, | | |
| MMP9 | | | CD44 (MT1-MMP) | | |
| | | | {3} | | |
| MMP1, 2, 3 | Cardiovascular | Over- | Type I Collagen | TIMP-2 {2} | {1}, {2}, {3}, |
| and 9 | diseases | expressed | (MMP1); | | {6}, {18} |
| | | | Fibronectin, E- | | |
| | | | Cadherin, Basement | | |
| | | | membrane (MMP3) | | |
| | | | {3}; | | |
| | | | AGFSGPLGMWSA | | |
| | | | GSFG (MMP2) | | |
| | | | {18} | | |
| MMP2, 3 and 9 | Cerebrovascular | Over- | GGPLG~LWAGG | | {1}, {3}, |
| | Diseases | expressed | (MMP2 and MMP9) | | {4}, {7} |
| | | | {1}; GPLGVRC | | |
| | | | (MMP2) {2}; | | |
| | | | Basement | | |
| | | | membrane (MMP3) | | |
| | | | {3}; CGLDD | | |
| | | | (MMP2,9) {4}; | | |
| | | | LMWP (ALMWP, | | |
| | | | E10-PLGLAG- | | |
| | | | VSRRRRRRGGRR | | |
| | | | RR) (MMP2) {7} | | |
| MMP1, 2 and | Pulmonary | Over- | Type I Collagen | cyclic peptide | {1}, {2}, {3}, |
| 9; MMP3, 11 | Diseases, | expressed | (MMP1); | inhibitor | {4}, {6}, |
| and 14 {4}; | small-cell lung | | Chondroitin | (CTT), | {17} |
| MMP13 | cancer {4} | | sulphate | CTTHWGFTL | |
| | {17}; non- | | proteoglycan | C of both | |
| | small cell lung | | (MMP2); ICAM-1, | MMP2 and | |
| | cancer {4} | | IL-2Ra (MMP9) | MMP9 {2} | |
| | | | {3} | | |
| MMP2 and | Ocular | Over- | Fibronectin | | {1}, {3} |
| MMP9 | Diseases | expressed | (MMP2); | | |
| | | | Plasminogen | | |
| | | | (MMP9) {3} | | |
| MMP1, 3, 7, 9, | GI diseases, | Over- | Plasminogen | | {1}, {3}, |
| 10, 12 and | cancers | expressed | (MMP1, 3, 7, 9, 12) | | {4}, {7} |
| MT1-MMP | | | {3}; GPLGIAGQ | | |
| (MMP14) | | | (MMP2) {4}; GLY- | | |
| | | | PRO-LEU-GLY- | | |
| | | | ILE-ALA-GLY- | | |
| | | | GLN (MMP2, 3, 7 | | |
| | | | and 9) {7} | | |
| MMP8 | Oral Diseases | Over- | Type I Collagen {3} | | {1}, {3} |
| (Collagenases) | | expressed | | | |
| MMP11 | Breast Cancer | Over- | IGFBP-1 (MMP11) | | {4}, {3} |
| | | expressed | {3} | | |
| Urokinase | Angiogenesis, | Over- | KLDLKLDLKLDL | | {1}, {4}, |
| Plasminogen | Tissue | expressed | (uPA) {4}; Ser-Gly- | | {19} |
| Activator | remodeling, | | Arg-Ser-Ala {19} | | |
| (uPA) | Rheumatoid | | | | |
| | Arthritis | | | | |
| A disintegrin | Breast cancer, | Over- | | TIMP1-4 | {5}, {6} |
| and | Bladder | expressed; | | | |
| metalloproteinase- | cancer, Lung | Decreased | | | |
| 12 | Adenocarcinoma, | levels in brain | | | |
| (ADAM12) | Brain | tumors | | | |
| | tumors, | | | | |
| | Asthma | | | | |
| Cysteine | Esophageal | Over- | PHE-LYS-PHE- | Thyropins, | {8}, {9}, |
| Cathepsin B | cancer, Liver | expressed | LEU (FKFL-CathB) | Precursor | {10}, {11}, |
| and D | cancer, brain | | {9}; GGGF (Cath | peptide, Serpin | {12}, {14} |
| | | | B) {10}; | family, | |
| | | | CRRGGKKGGKK | Cystatin | |
| | | | RK (CathB) {11} | family, a2- | |
| | | | | Macroglobulin, | |
| | | | | Cytotoxic T | |
| | | | | lymphocyte | |
| | | | | antigen- 2b; | |
| | | | | Cystatin A, B | |
| | | | | and C (CathB) | |
| | | | | {12} {14} | |
| Cysteine | Gastric | Over- | PMGLP (Cath S) | Thyropins, | {8}, {10}, |
| Cathepsin D, | carcinoma; | expressed | {10} | Precursor | {15} |
| E, S and X | Arthritis, | | | peptide, Serpin | |
| | Asthma, | | | family, | |
| | Diabetes and | | | Cystatin | |
| | Obesity | | | family, a2- | |
| | (CathS) {15} | | | Macroglobulin, | |
| | | | | Cytotoxic T | |
| | | | | lymphocyte | |
| | | | | antigen- 2b; Z- | |
| | | | | Phe- | |
| | | | | Leu_COCHO | |
| | | | | (CathS) {15} | |
| Cysteine | Colorectal | Over- | PHE-LYS-PHE- | Thyropins, | {8}, {9}, |
| Cathepsin B, | Carcinoma, | expressed | LEU (FKFL- | Precursor | {10}, {11}, |
| D, L, E, H and | Pancreatic | | CathB) {9}; | peptide, Serpin | 12}, {13}, |
| K | cancer, brain | | GGGF (Cath B) | family, | {14}, {16} |
| | cancer, | | {10}; | Cystatin | {17} |
| | prostate | | CRRGGKKGGKK | family, a2- | |
| | cancer, | | RK (CathB) {11} | Macroglobulin, | |
| | ovarian | | | Cytotoxic T | |
| | cancer, lung | | | lymphocyte | |
| | diseases {16} | | | antigen- 2b; | |
| | | | | Cystatin A, B | |
| | | | | and C (CathB) | |
| | | | | {12} {14} | |

*MMP—Matrix Metalloproteinases;
TIMPs—tissue inhibitors of metalloproteases;
Cath—Cathepsin
Table 9 details examples of cancer-specific and disease-specific overexpressed proteases and associated cleavable peptide sequences for inclusion within nanoparticle polypeptides.

### REFERENCES (PROTEOLYTIC ENZYMES)

1. Matrix metalloproteases: Underutilized targets for drug delivery Deepali G. Vartak and Richard A. Gemeinhart
2. Matrix-metalloproteinases as targets for controlled delivery in cancer: an analysis of upregulation and expression Kyle J. Isaacson, M Martin Jensen, Nithya B. Subrahmanyam, and Hamidreza Ghandehari
3.Matrix Metalloproteinases and Tissue Inhibitors of Metalloproteinases Structure, Function, and Biochemistry Robert Visse, Hideaki Nagase
4. Peptides in Cancer Nanomedicine: Drug Carriers, Targeting Ligands and Protease Substrates Xiao-Xiang Zhang, Henry S. Eden, and Xiaoyuan Chen
5. A Disintegrin and Metalloproteinase-12 (ADAM12): Function, Roles in Disease Progression, and Clinical Implications. Erin K Nyren-Erickson, Justin M Jones, D. K Srivastava, and Sanku Mallik
6. Matrix Metalloproteinase Inhibitors as Investigational and Therapeutic Tools in Unrestrained Tissue Remodeling and Pathological Disorders. Jie Liu and Raouf A. Khalil
7. Enzyme-Responsive Nanomaterials for Controlled Drug Delivery. Quanyin Hua, Prateek S. Katti and Zhen Gu
8. Cathepsins in digestive cancers. Siyuan Chen, Hui Dong, Shiming Yang and Hong Guo
9. Cathepsin B-sensitive polymers for compartment-specific degradation and nucleic acid release. David S. H. Chu, Russell N Johnson and Suzie H. Pun
10. 177Lu-labeled HPMA Copolymers Utilizing Cathepsin B and S Cleavable Linkers: Synthesis, Characterization and Preliminary In Vivo Investigation in a Pancreatic Cancer Model. Sunny M. Ogbomo, Wen Shi, Nilesh K Wagh, Zhengyuan Zhou, Susan K Brusnahan, and Jered C. Garrison
11. Peptide-mediated core/satellite/shell multifunctional nanovehicles for precise imaging of cathepsin B activity and dual-enzyme controlled drug release. Fenfen Zheng, Penghui Zhang, Yu Xi, Kaikai Huang, Qianhao Min and Jun-Jie Zhu
12. Cathepsin B as a Cancer Target Christopher S. Gondi and Jasti S. Rao
13. Cathepsin L targeting in cancer treatment. Dhivya R. Sudhan and Dietmar W Siemann
14. Cathepsin B: Multiple roles in cancer Neha Aggarwal and Bonnie F. Sloane
15. Cathepsin S: therapeutic, diagnostic, and prognostic potential. Richard D. A. Wilkinson, Rich Williams, Christopher J. Scott and Roberta E. Burden
16. Specialized roles for cysteine cathepsins in health and disease. Jochen Reiser, Brian Adair, and Thomas Reinheckel
17. Expression of Proteolytic Enzymes by Small Cell Lung Cancer Circulating Tumor Cell Lines. Barbara Rath, Lukas Klameth, Adelina Plangger, Maximilian Hochmair, Ernst Ulsperger, Ihor Huk, Robert Zeillinger and Gerhard Hamilton.
18. Enzyme-responsive multistage vector for drug delivery to tumor tissue. Yu Mia, Joy Wolframa, Chaofeng Mu, Xuewu Liu, Elvin Blanco, Haifa Shena, and Mauro Ferrari.
19. Enzyme-Responsive Liposomes for the Delivery of Anticancer Drugs. Farnaz Fouladi, Kristine J. Steffen, and Sanku Mallik
   For the targeting ligands of the nanoparticle, we need to compile amino acid sequences of ligands and their respective cell surface receptors. These will be the ligands with electrostatic anchors for targeted delivery. The associated database can be found at http://mips.helmholtz-muenchen.de/HSC/. Associated paper can be found at https://www.ncbi.nlm.nih.gov/pubmed/23936191.

| | | Ligand or Co- | |
|---|---|---|---|
| HSC type | Marker | localizing protein | Comment |
| HSC (CD150+ CD48− | | sinusoidal | HSCs within the mobilized spleen are |
| CD41− Lin−) | | endothelium | associated with sinusoidal endothelial cells. |
| | | (MECA-32+) | [Method: Immunofluorescence analysis, |
| | | | MECA-32/CD150, CD48, CD41, Lin−; |
| | | | cyclophosphamide/G-CSF-mobilized spleen] |
| LT-HSC | Vcam1 | Itga4 | VCAM1 and ESAM are related adhesion |
| | | | molecules upregulated in LT-HSC. VCAM1 |
| | | | interaction with integrin alpha4beta1 mediates |
| | | | cell-cell interactions in multiple cell types, and |
| | | | both VCAM1 and integrin alpha4beta1 have |
| | | | been implicated in HSC homing to the bone |
| | | | marrow. [Method: cited information, PMID: |
| | | | 7568190] |
| LT-HSC | DCC | Robo4 | Robo4 can interact directly with DCC, a |
| | | | homolog of Neogenin which is also |
| | | | upregulated in LT-HSC over MPP. In other |
| | | | systems, Neogenin and DCC are implicated in |
| | | | cell adhesion, polarity, and migration, and are |
| | | | receptors for the Netrin family of |
| | | | chemoattractants. [Method: cited information] |
| LT-HSC | ApoE | App | Amyloid beta precursor protein (App) is a |
| | | | heparin-binding cell adhesion molecule that |
| | | | interacts with two of the extracellular matrix |
| | | | molecules upregulated in LT-HSC, ApoE and |
| | | | biglycan. [Method: cited information] |
| LT-HSC | biglycan | App | Amyloid beta precursor protein (App) is a |
| | | | heparin-binding cell adhesion molecule that |
| | | | interacts with two of the extracellular matrix |
| | | | molecules upregulated in LT-HSC, ApoE and |
| | | | biglycan. [Method: cited information] |
| HSC | Nedd4 | Grb10 | Grb10 interacts with Nedd4, a ubiquitin protein |
| | | | ligase robustly expressed in HSC. [Method: |
| | | | cited information] |
| quiescent HSC | MPL | Thpo | MPL is the receptor for the ligand |
| | | | thrombopoietin. MPL signaling upregulated |
| | | | b1-integrin and cyclin-dependent kinase |
| | | | inhibitors in HSCs. Furthermore, inhibition and |
| | | | stimulation of THPO/MPL pathway by |
| | | | treatments with anti-MPL neutralizing |
| | | | antibody, AMM2, and with THPO showed |
| | | | reciprocal regulation of quiescence of LT-HSC |
| | | | [Method: cited information, 7605981] |
| HSC (Tie2+ SP, KSL) | | endosteum | Tie2+ HSCs specifically localized to the |
| | | | endosteal surface of adult BM. [Method: 5-FU |
| | | | treatment, BrdU labeling, |
| | | | immunohistochemical staining (Tie2/TOTO3), |
| | | | (Tie2/BrdU)] |
| HSC | LNK | Jak2 | Lnk directly binds to phosphorylated tyrosine |
| | | | residues in JAK2 following TPO stimulation |
| | | | [Method: TPO treatment for 10 min, flag- |
| | | | tagged coimmunoprecipitation] |
| HSC | Cxcr4 | Cxcl12 | The primary physiologic receptor for the |
| | | | chemokine CXCL12 is CXCR4. CXCL12- |
| | | | CXCR4 signaling is essential for |
| | | | hematopoiesis. [Method: cited information |
| | | | [PMID: 9634238] |
| HSC (CD135− KSL) | | N-cadherin+ | GFP+ HSCs directly attached to N-cadherin+ |
| | | | pre-osteoblastic cells. However, not all GFP+ |
| | | | HSCs were close to N-cadherin+ cells, |
| | | | indicating the existence of additional N- |
| | | | cadherin− niche components. [Method: |
| | | | immunohistochemistry, co-staining with N- |
| | | | cadherin and GFP] |
| HSC | Itgb1 | Opn | HSCs adhere to Opn via beta 1 integrins. |
| | | | [Method: Calcein-AM-labeled BM CD34+ |
| | | | cells were assayed for their ability to attach to |
| | | | GST fusion-tagged full-length human Opn, |
| | | | specific {beta}1 integrin-blocking antibody |
| | | | P5D2] |
| HSC (Lin− | Itga9 | Itgb1 | Human Lin− CD34+ CD38− CD90(bright) cells |
| CD34+ CD38− | | | express alpha9 integrin, which interacts with |
| CD90(bnght)) | | | beta1 integrin to fonn a functional heterodimer. |
| | | | [Method: FACS] |
| LSKCD34− HSC | Vcam1 | VLA-4 | VCAM-1 is a major receptor of LSKCD34− |
| | | | hematopoietic cells on endothelial cells. Its |
| | | | major ligand is the integrin very late antigen 4 |
| | | | (VLA-4) [Method: cited information, PMID: |
| | | | 7568190] |
| HSC | Hspa8 | Ccnd1 | Hsc70 directly interacts with cyclin D1 and |
| | | | accelerates its binding to CDK4/6 during the |
| | | | G0/G1-S transition. [Method: cited |
| | | | information] |
| HSC | Bmi1 | Akt | Bmi1 interacts with Akt, which is part of the |
| | | | PI3K-Akt signaling pathway. [Method: HeLa |
| | | | cells, co-immunoprecipitation] |

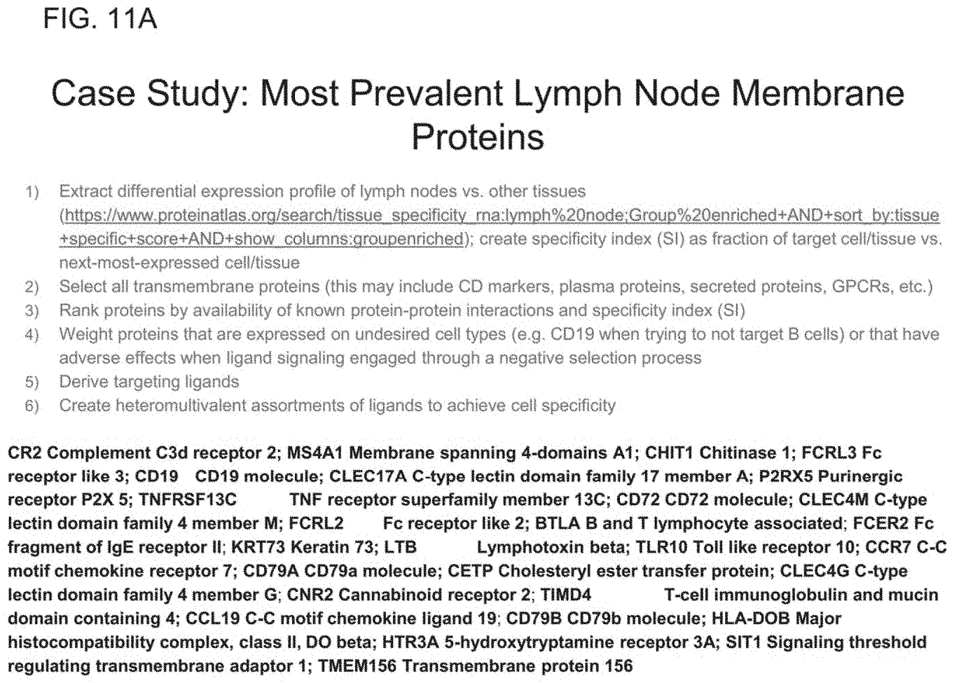

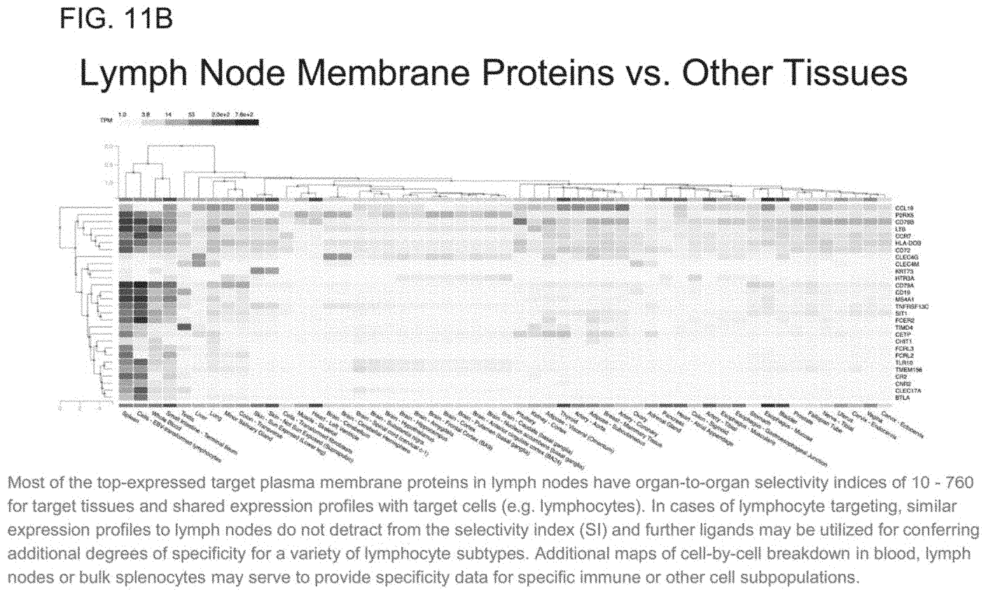

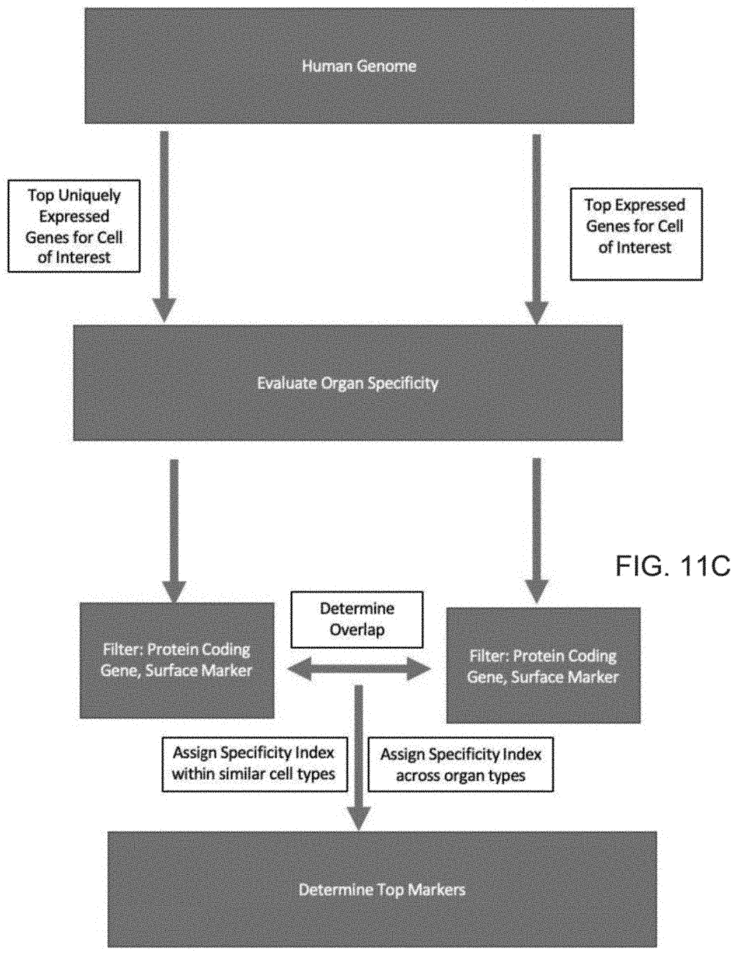

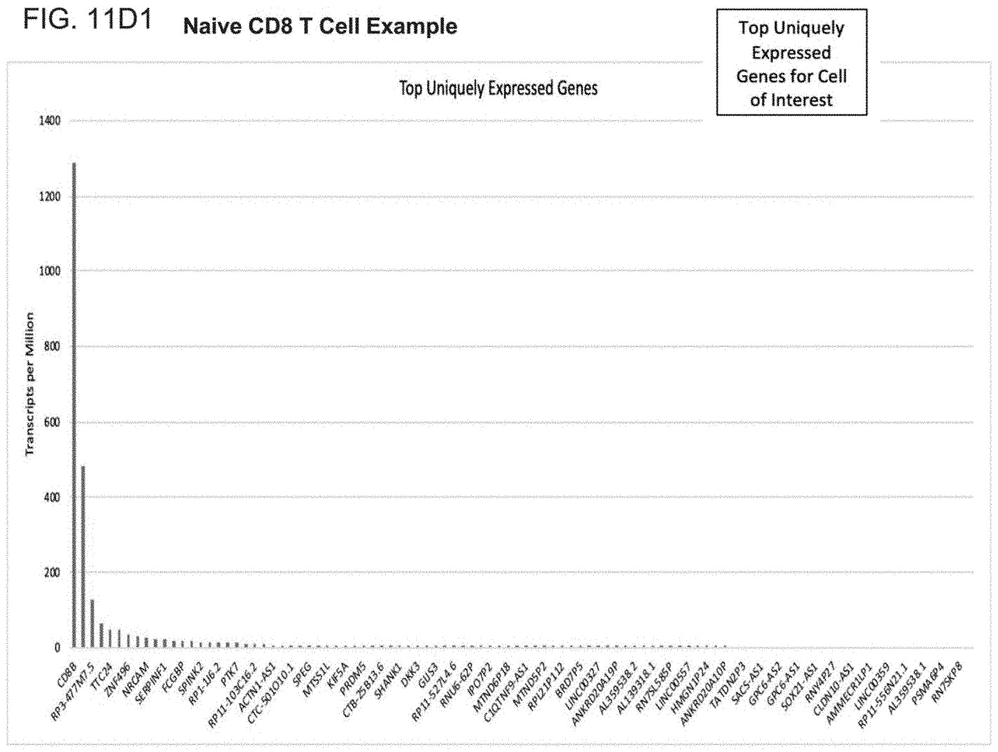

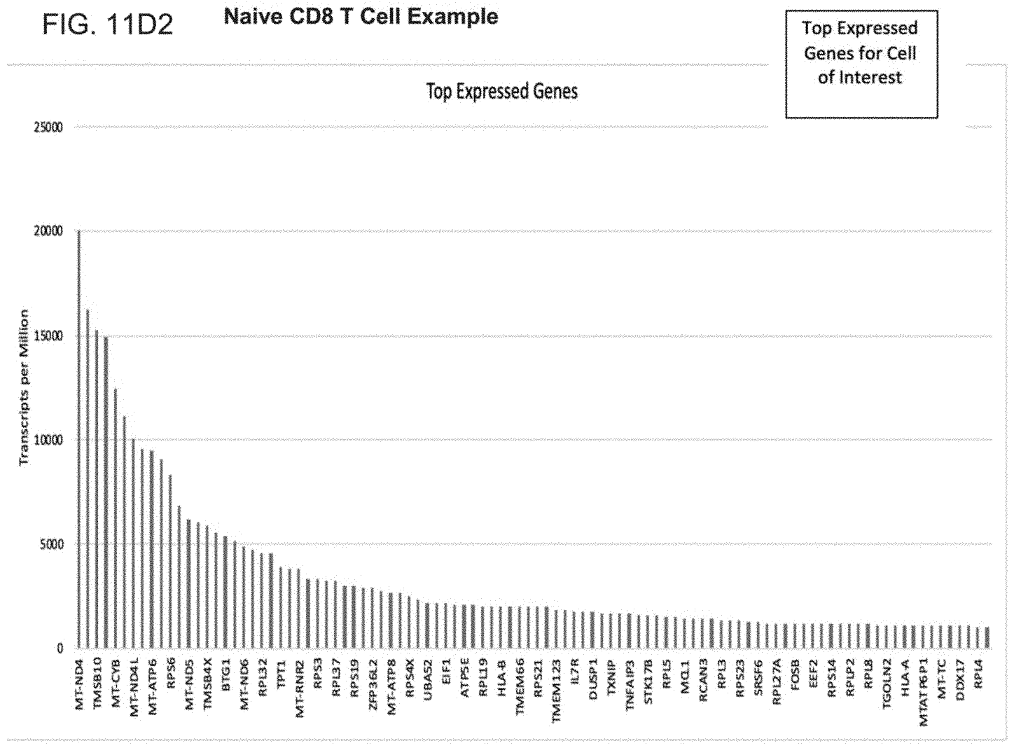

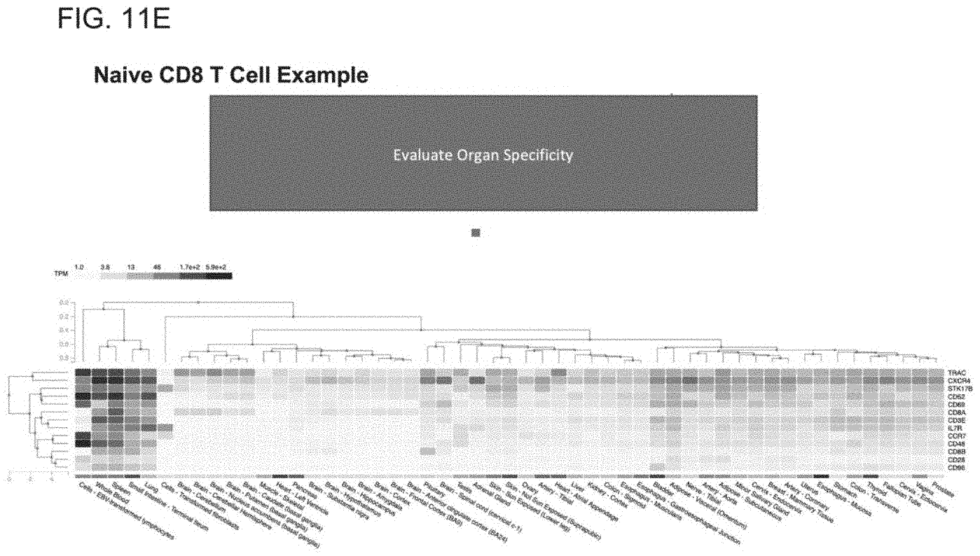

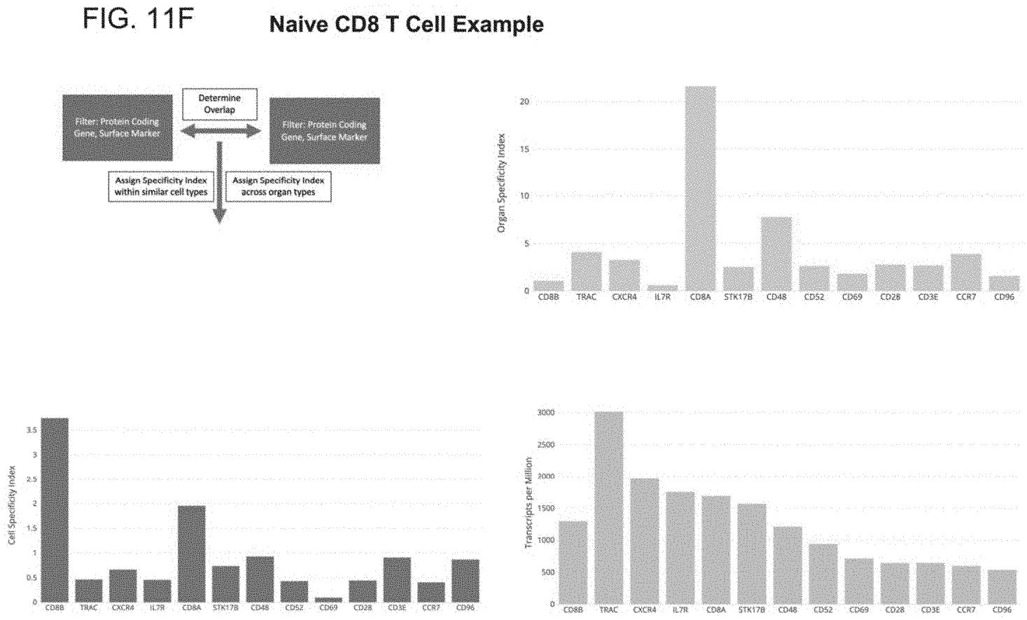

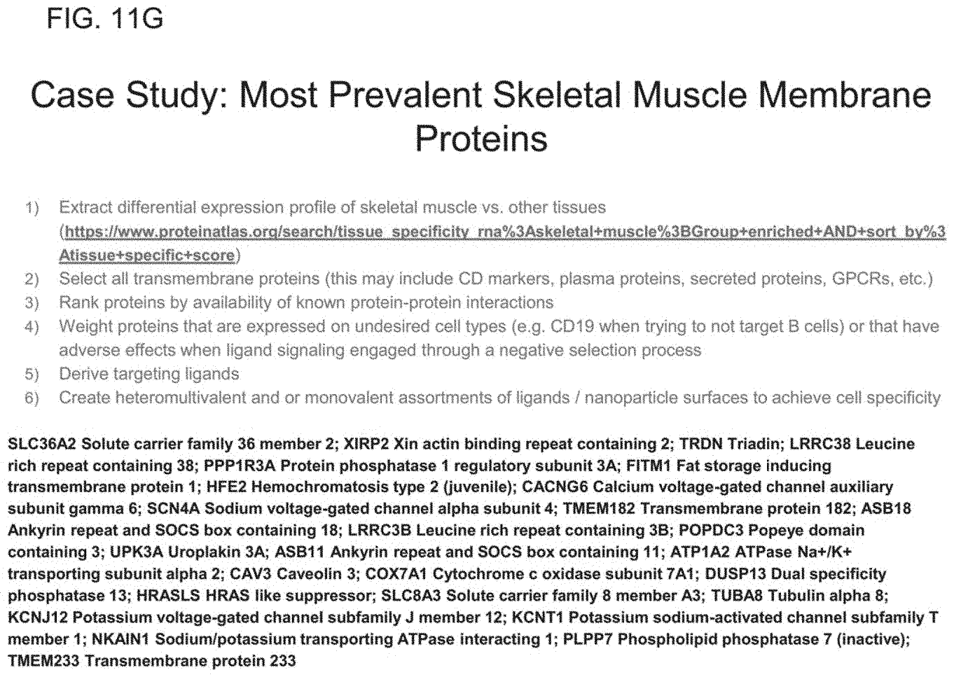

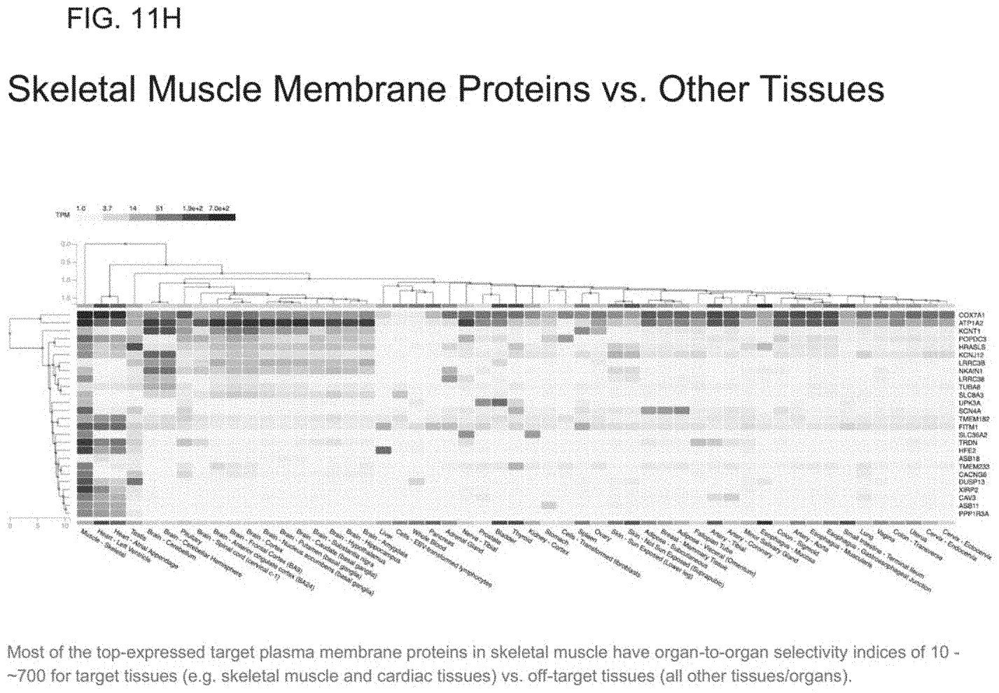

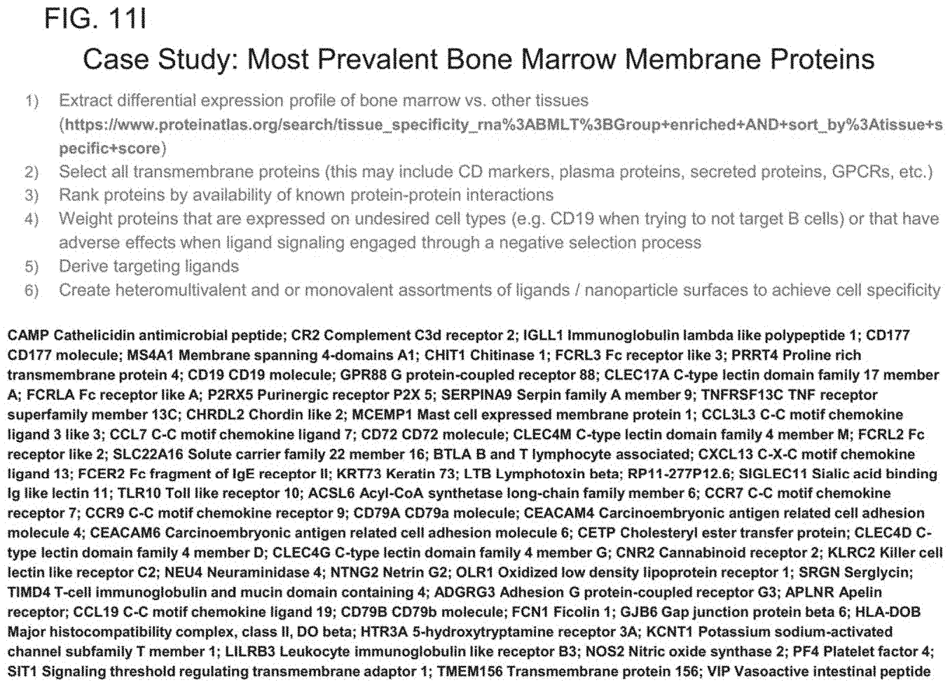

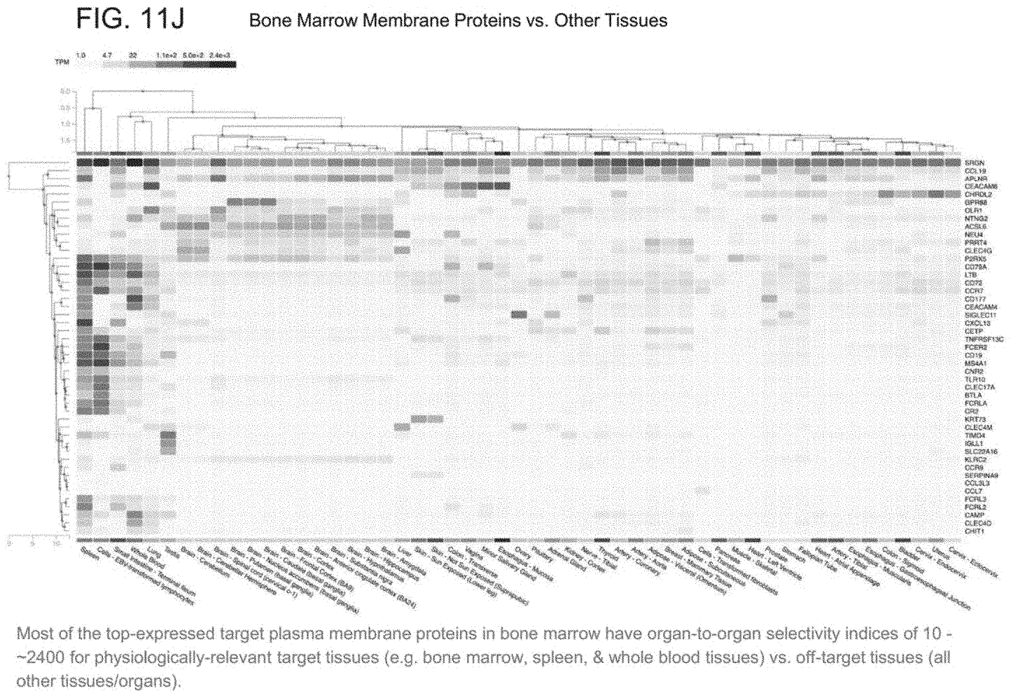

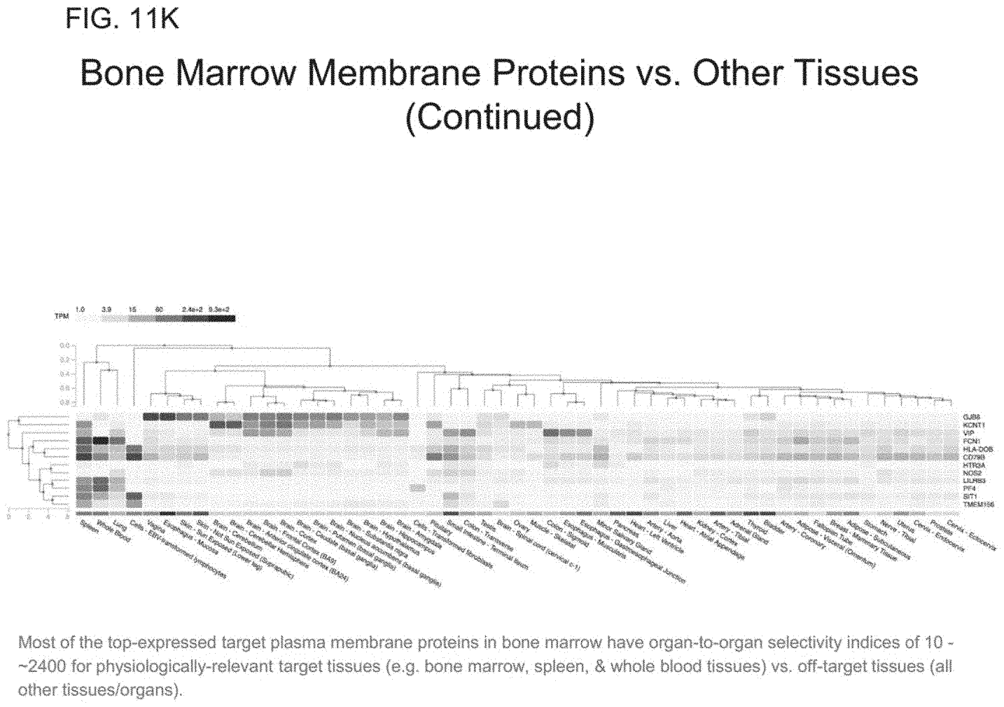

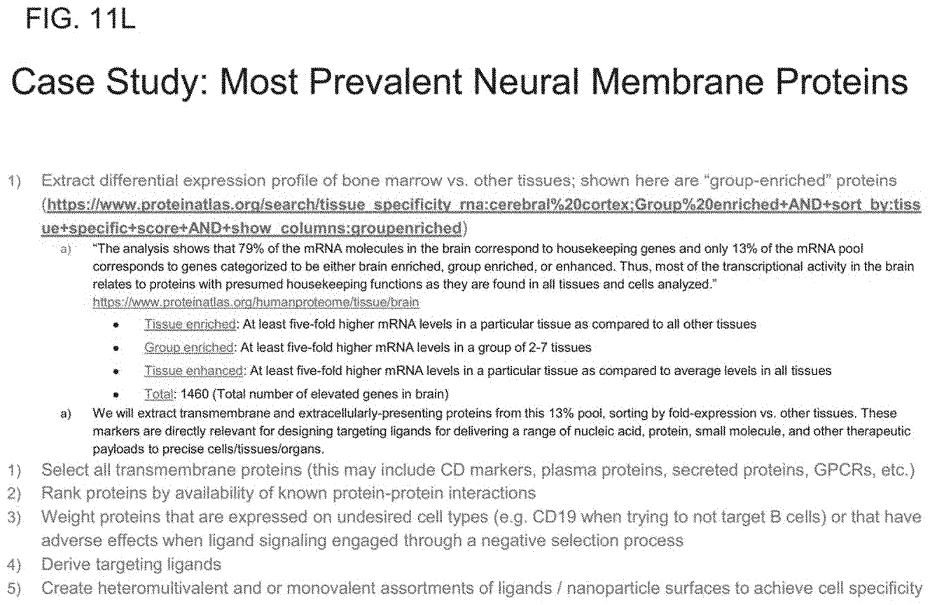

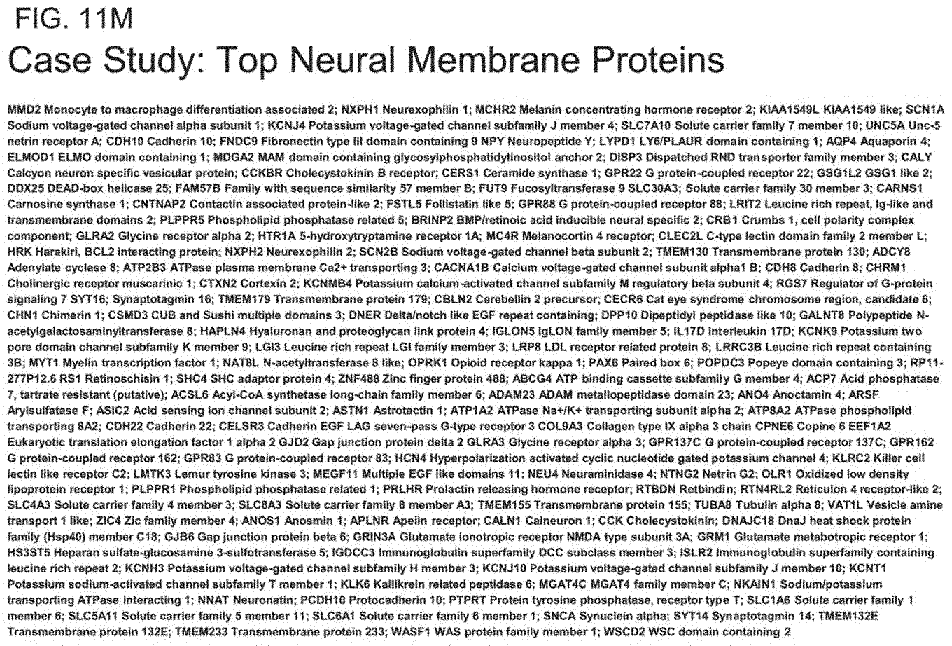

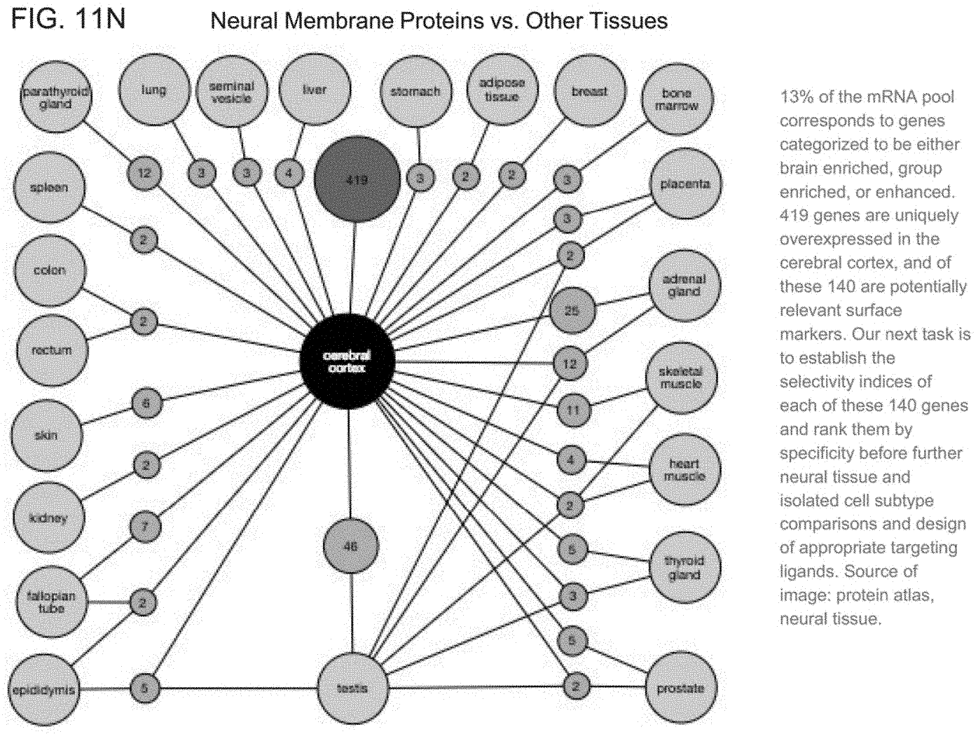

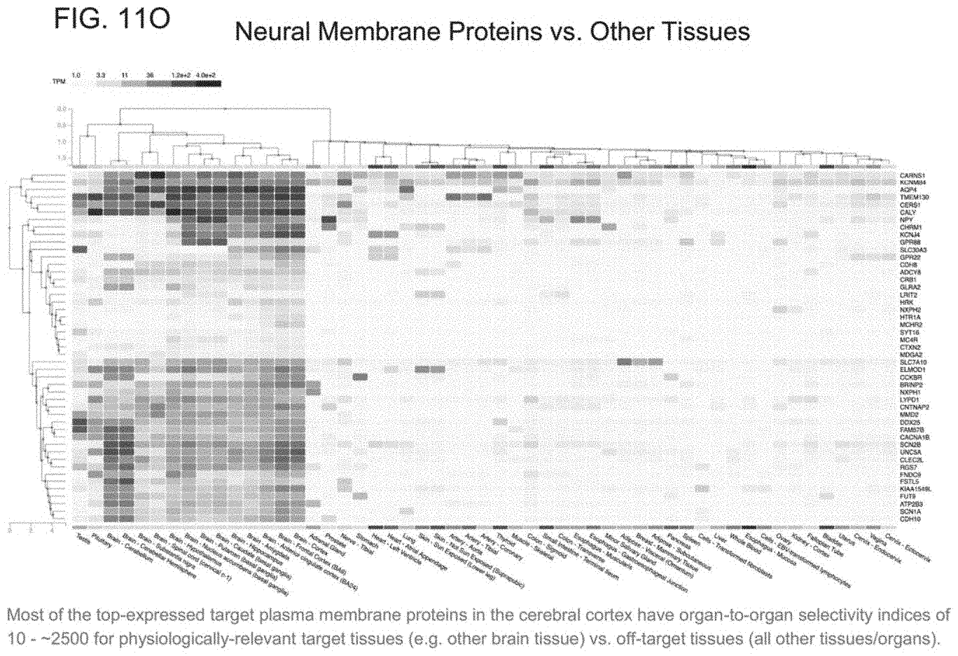

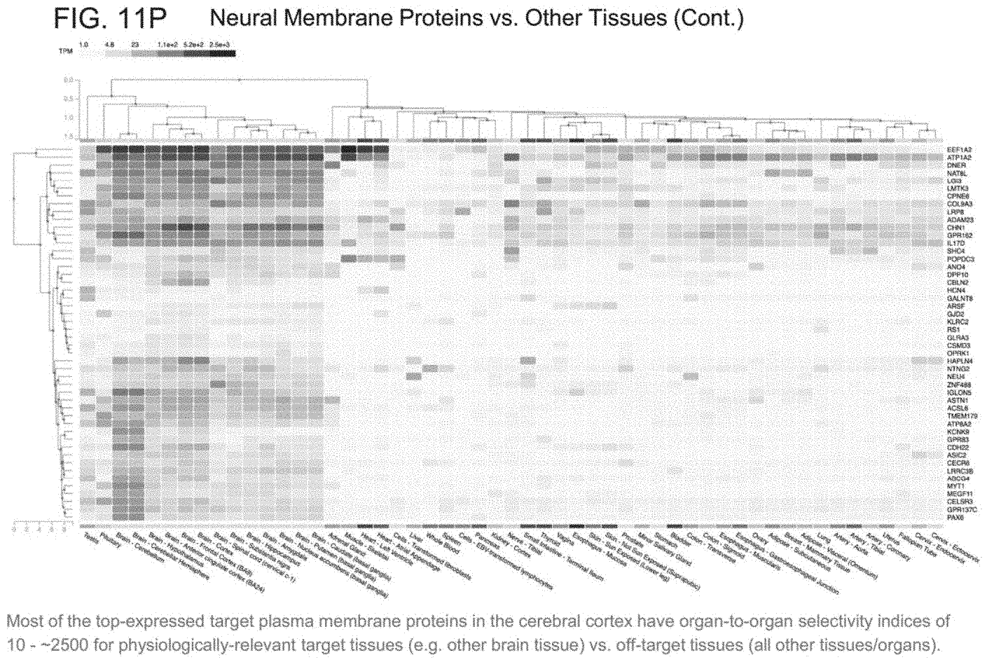

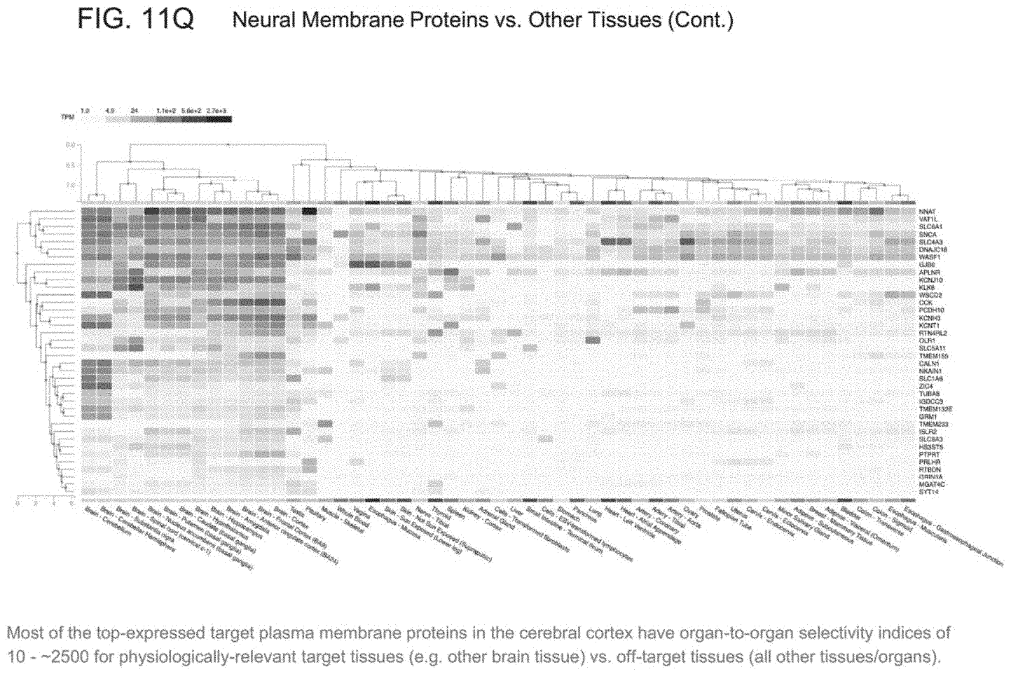

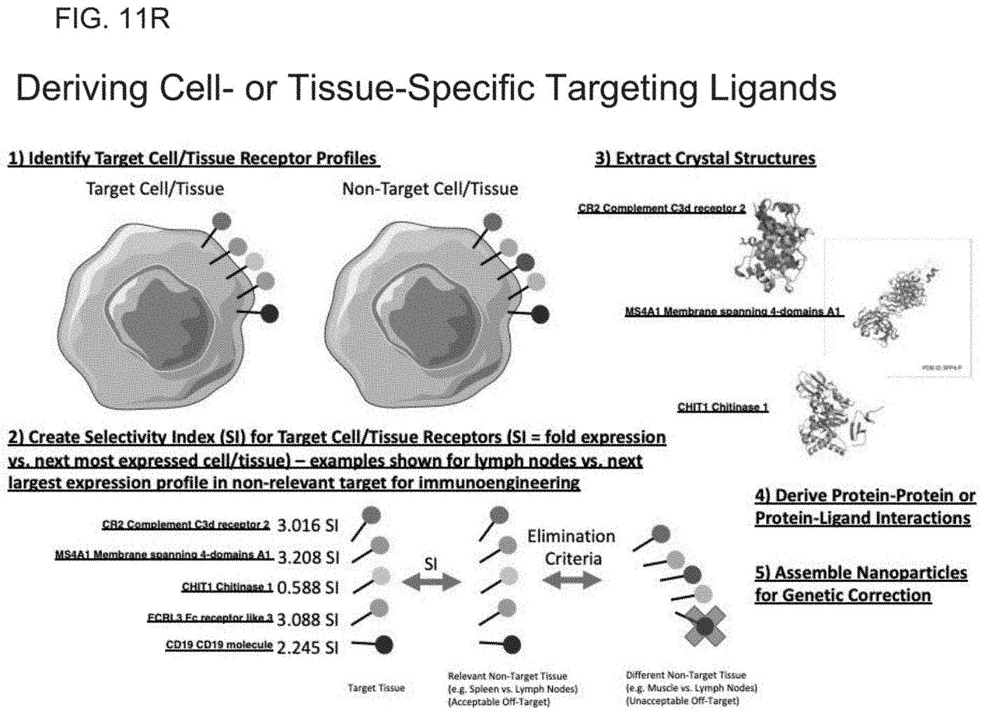

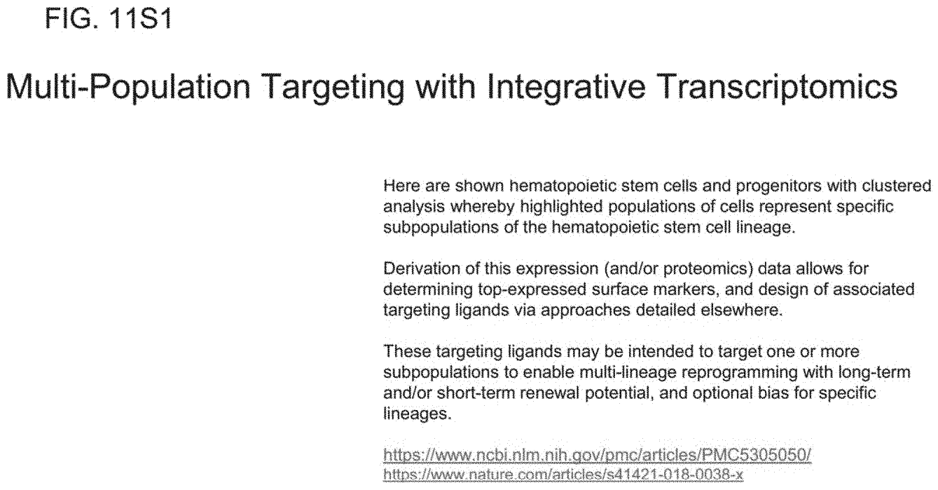

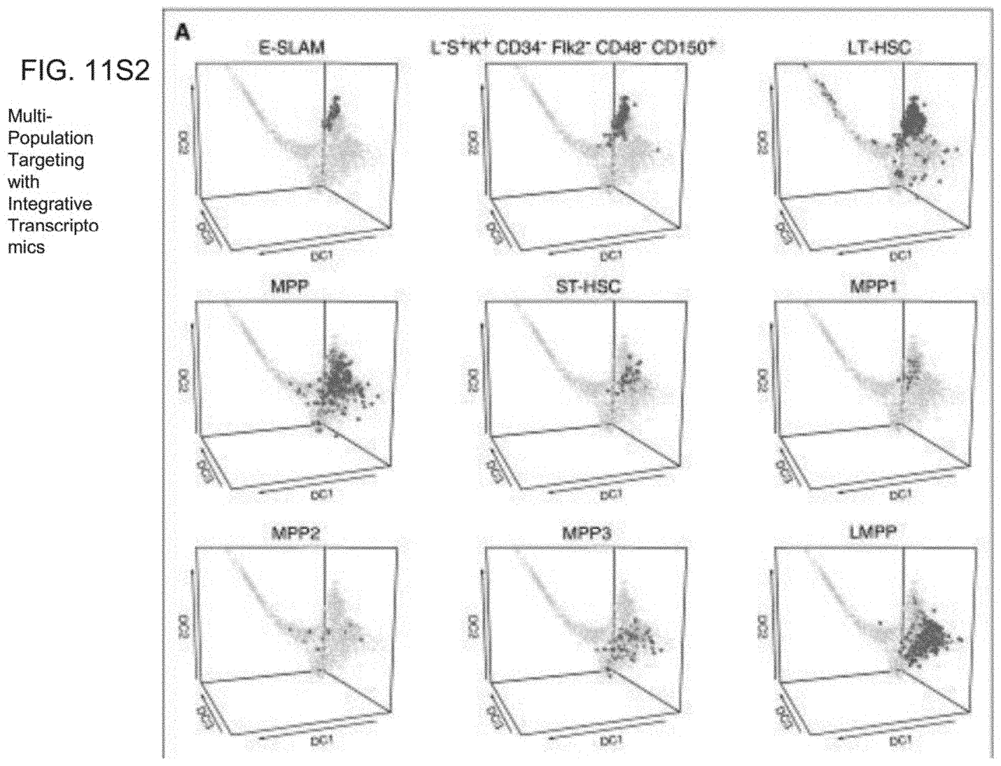

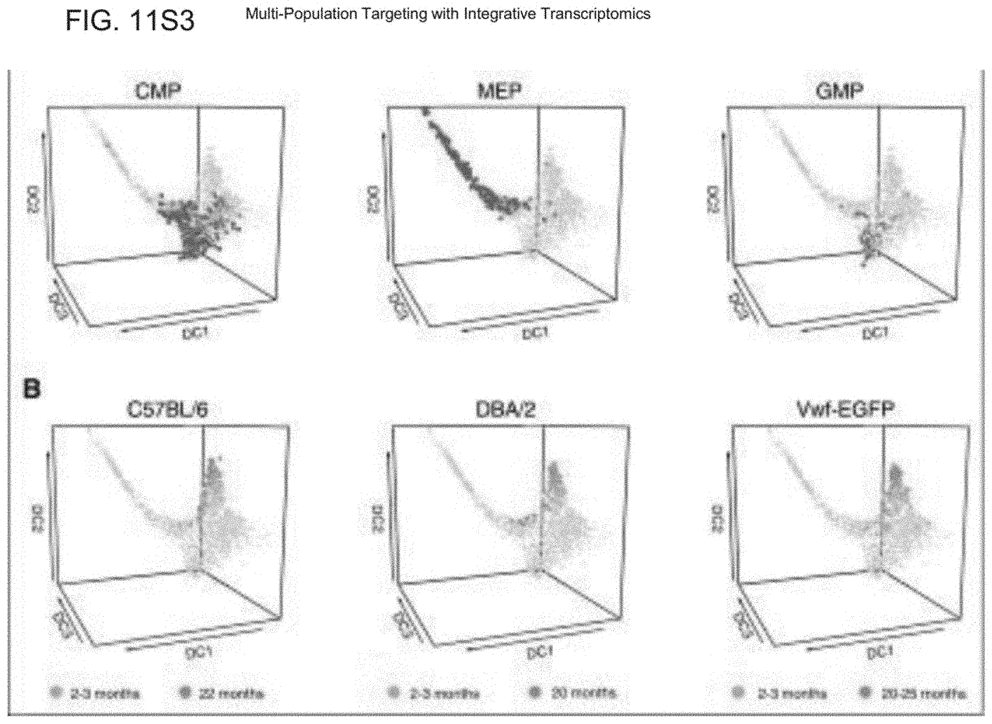

Table 10 depicts cell targeting ligands for hematopoietic stem cells (FIGS. 11S1-3).

Any combination of the above personalized techniques can be used. For example, diagnostic information can be used to select a targeting ligand (and/or desired cell type to target), a promoter, and cargo. On the other hand, a more generalized cargo can be delivered in a personalized (diagnostically responsive) way by delivering the cargo using a delivery vehicle (e.g., a nanoparticle) that has a targeting ligand this is personalized. Likewise, a specific personalized cargo (e.g., a gene-editing cargo that edits a T cell receptor) can be delivered using a delivery vehicle that does not include a personalized targeting ligand—e.g., a delivery vehicle such as a nanoparticle can be delivered by local inject such as intratumoral injection. A combination of promoters and protease-specific sequences may also be utilized to increase cell, tissue, organ and/or cancer-specific release and activity of a given payload.

In some cases, a subject method is not molecularly tailored to a particular individual based on diagnostic information (e.g., genotype/phenotypic evaluation). For example, localization can in some cases be achieved via direct local injection (e.g., into a tumor). In some cases, delivery is not personalized (is not diagnostically responsive). For example, in some cases a subject delivery vehicle (e.g., a nanoparticle) is delivered without using a targeting ligand, promoter or protease domain that was designed based on the patient's profile. For example, in some cases a delivery vehicle is delivered via passive delivery (e.g., systemic delivery or local delivery such as injection) so that it accumulates in a target tissue such as a tumor.

### II. Secreted Payloads and Secretomimetic Ligand Coatings

The tumor (or organ/tissue) microenvironment's pathophysiology and immunological milieu also present a set of hurdles for successful immunotherapy and/or nanoparticle targeting. The tumor microenvironment (TME) is a complex and dynamic circuit of malignant and non-malignant cell interactions. Due to the TME's hypoxic and inflammatory setting, antigen presenting cells in the TME can fail to activate the immune system. Malignant cells are also known to recruit T regulatory cells and myeloid derived suppressor cells as well as promote production of IL-10, vascular endothelial growth factor, indoleamine 2,3-dioxygenase, TGF-0, and other immunosuppressive chemokines. Delivery vehicles such as nanoparticles of this disclosure can be used to suppress the production of these and other factors through delivery of siRNA or miRNA that target the immunosuppressive signals such as chemokines. On the other hand, delivery vehicles (such as nanoparticles) of this disclosure can be used to deliver, as a payload, a nucleic acid that encodes a secreted protein, e.g., pro-inflammatory signs such as a cytokine.

In some embodiments, delivery of the payload results in expression and secretion of a protein of interest (a protein such as a cytokine that modulates the local tumor microenvironment after secretion). In other embodiments, “secretomimetic” ligands may confer favorable characteristics to nanoparticles designed to function in a specific secretome environment (e.g. FIGS. 18C, 18I). Thus, in some cases the payload or ligand is a secreted protein of interest (e.g., an immune signal such as a cytokine) (or a nucleic acid encoding same). In some cases a delivery vehicle that delivers a secreted payload (or a nucleic acid encoding same) is targeted to express in a particular cell and/or tissue, e.g., a cancer cell/tissue. In some cases, for example in some cases where the secreted protein is a cytokine, the secreted protein influences the microenvironment of the targeted cell(s) (e.g., a tumor microenvironment). Examples of proteins that can be used include, but are not limited to those presented in Table 2 (including any variants thereof that retain their function to stimulate the immune system). Other proteins and protein fragments may not necessarily be immunostimulatory, but may mimic an ideal microenvironment for targeting a specific tissue (e.g. FIG. 20B depicting a lung-derived protein with mucoadsorptive properties). In some cases, the payload includes a secreted cytokine (or a nucleic acid encoding it). In some cases the secreted cytokine is selected from: IL-2, IL-7, IL-12, IL-15, IL-21, and IFN-gamma. In some cases the secreted cytokine is selected from: IL-2, IL-7, IL-15, IL-21, and IFN-gamma. In some cases the secreted cytokine is not IL-12. Driving modulation of organ, tissue, cell or cancer expression of a target cytokine, chemokine, or corresponding receptor can have manifold effects on inflammatory, autoimmune, or immunosuppressive microenvironments. Other cytokines and chemokines, and their immune cell subpopulation effects (as would be relevant for upregulating or downregulating a particular immune population's activity in a specific environment following various cytokine-expressing delivery approaches), can be found here:

Cytokine function table
Interleukin

| | | | | | Cytokine |
|---|---|---|---|---|---|
| | Cytokine | Cytokine | Cytokine | | Disease |
| Cytokine | Receptor | Source | Targets | Cytokine Main Function | Association |
| IL-1α; | IL1RI and | Macrophages, | Macrophages, | Inflammatory; promotes | ↑ = |
| IL-1b | IL1R-AcP | many others | thymocytes, | activation, costimulation, | inflammatory |
| | | | CNS, others | and secretion of cytokines | bone resorption; |
| | | | | and other acute-phase | gout; promotes |
| | | | | proteins; pyrogenic | Th17 response |
| IL-1ra | Soluble | | | IL-1ra and the soluble | |
| (antagonist) | decoyreceptor: | | | decoy receptor complex | |
| | IL1RII and | | | inhibit IL-1-mediated | |
| | IL1R-AcP | | | inflammatory responses | |
| IL-2 | IL2Rα, | T cells | T, B, NK cells, | Proliferation; enhancement | ↓ = |
| | IL2Rb, and | | and macrophages | of cytotoxicity, IFNγ | lymphoproliferative |
| | IL2Rγ | | | secretion, and antibody | disease and |
| | | | | production | susceptibility to |
| | | | | | autoimmune |
| | | | | | disease; reduced |
| | | | | | Treg |
| | | | | | development. ↑ = |
| | | | | | reduced Th17 |
| | | | | | development. |
| IL-3 | IL3Rα and | T cells, mast | Hematopoietic | Differentiation and survival | |
| | IL3Rb | cells, | progenitors, | of lymphoid and myeloid | |
| | | eosinophils | macrophages, | compartment | |
| | | | mast cells | | |
| IL-4 | IL4Rα and | T cells, mast | T cells, B cells, | Proliferation; differentiation | ↓ = susceptibility |
| | IL2Rγ or | cells | macrophages, | of Th2; promotes IgG and | to extracellular |
| | IL4Rα and | | monocytes | IgE production; inhibits | pathogens and |
| | IL13Ra1, | | | cell-mediated immunity and | decreased |
| | IL13Ra2 | | | Th17 development | response to |
| | | | | | allergens. ↑ = |
| | | | | | allergic asthma. |
| IL-5 | IL5Rα and | Th2 cells | Eosinophils, B | Proliferation and activation; | ↓ = eosinophil |
| | IL3Rb | | cells | hallmark of Th2 effector | and B-1 cell |
| | | | | cells | deficiency. ↑ = |
| | | | | | allergic asthma. |
| IL-6 | IL6Rα and | Macrophages, | Wide variety of | Inflammatory and | ↓ = deficient |
| | gp130 | T cells, | cells: B cells, T | costimulatory action; | innate immunity |
| | | fibroblasts, | cells, | induces proliferation and | and acute- phase |
| | | and others | thymocytes, | differentiation; synergizes | responses, |
| | | | myeloid cells, | with TGFb to drive Th17 | lymphopenia |
| | | | osteoclasts | | |
| IL-7 | IL7Rα and | Thymic | B cells, T cells, | Homeostasis, differentiation, | ↓ = severe |
| | IL2Rγ | stromal cells, | thymocytes | and survival | combined |
| | | bone marrow, | | | immune |
| | | and spleen | | | deficiency |
| | | | | | (SCID) |
| IL-9 | IL9R and | T cells (Th2) | T cells, mast | Proliferation; promotes Th2 | |
| | IL2Rγ | | cells, neutrophils, | cytokine secretion | |
| | | | epithelial cells | | |
| IL-10 | IL10R1 and | Differentiated | Macrophages, T | Immune suppression; | ↓ = immune |
| | IL10R2 | T helper cells, | cells, dendritic | decreases antigen | pathology due to |
| | | Tregs, B cells, | cells, B cells | presentation and MHC class | uncontrolled |
| | | dendritic | | II expression of dendritic | inflammation. ↑ = |
| | | cells, others | | cells; downregulates | inhibits sterile |
| | | | | pathogenic Th1, Th2, and | immunity to |
| | | | | Th17 responses | some pathogens. |
| IL-11 | IL11Rα and | Stromal cells | Hematopoietic | Proliferation | ↑ = exacerbates |
| | gp130 | | stem cells, B | | airway diseases |
| | | | cells, | | |
| | | | megakaryocytes | | |
| IL-12 | IL12Rb1 and | Macrophage, | T cells, NK cells | Differentiation and | ↓ = impaired |
| (p35 + | IL12Rb2 | dendritic | | proliferation; promotes Th1 | Th1 responses |
| p40) | | cells, B cells, | | and cytotoxicity | and increased |
| | | neutrophils | | | susceptibility to |
| | | | | | intracellular |
| | | | | | pathogens |
| IL-13 | IL13Ra1, | T cells | B cells, | Goblet cell activation in | ↓ = impaired |
| | IL13Ra2 and | | macrophages, others | lung and gut; proliferation | Th2 responses to |
| | IL4Rα | | | and promotion of IgE | extracellular |
| | | | | production; regulation of | pathogens and |
| | | | | cell-mediated immunity | allergens. ↑ = |
| | | | | | exacerbates |
| | | | | | airway diseases. |
| IL-14 | Not defined | T cells | B cells | Promotion of B cell growth | |
| IL-15 | IL15Rα, | Broad | T cells, NK cells, | Proliferation and survival; | ↓ = deficiency in |
| | IL2Rb, and | expression in | epithelial cells, | cytokine production | NK cells and |
| | IL2Rγ | hematopoietic | others | | defective |
| | | cells | | | generation of |
| | | | | | memory T cells |
| IL-16 | Not defined | T cells, | CD4+ T cells | Recruitment of CD4+ T | |
| | | eosinophils, | | cells | |
| | | mast cells | | | |
| IL-17A | IL17RA | Th17 cells | Mucosal tissues, | Proinflammatory; | ↓ = susceptibility |
| | orIL17RC | and others | epithelial and | protective immunity in | to extracellular |
| | | | endothelial cells | lung; tight junction | pathogens ↑ = |
| | | | | integrity; promotes | exacerbates |
| | | | | mobilization of neutrophils | organ- specific |
| | | | | and cytokine production by | autoimmune |
| | | | | epithelial cells; promotes | inflammation |
| | | | | angiogenesis | |
| IL-17B | | Intestine and | | | |
| | | pancreas | | | |
| IL-17C | | thymus and | | | |
| | | spleen | | | |
| IL-17D | | T cells, | | | |
| | | smooth | | | |
| | | muscle cells, | | | |
| | | epithelial cells | | | |
| IL-17F | IL17RA or | Th17 cells | Mucosal tissues, | Similar function as IL-17A | Not well |
| | IL17RC | | epithelial and | but with 2 logs lower | defined. ↑ = |
| | | | endothelial cells | receptor affinity | increases |
| | | | | | neutrophil |
| | | | | | recruitment at |
| | | | | | high |
| | | | | | concentration. |
| IL-18 | IL18R and | Macrophages, | Th1 cells, NK | Proinflammatory; induction | ↓ = impairs Th1 |
| | IL18-R-AcP | others | cells, B cells | of IFNγ | responses |
| IL-19 | IL20R1 and | Monocytes, | Keratinocytes, | Proinflammatory | ↑ = psoriasis |
| | IL20R2 | others | other tissues | | |
| IL-20 | IL20R1 or | Monocytes, | Keratinocytes, | Proinflammatory | ↑ = psoriasis |
| | IL22R1 and | others | other tissues | | |
| | IL20R2 | | | | |
| IL-21 | IL21R and | Differentiated | T cells, B cells, | Proliferation of T cells; | |
| | IL2Rγ | T helper cells | NK cells, | promotes differentiation | |
| | | (Th2 and | dendritic cells | of B cells and NK | |
| | | Th17 subsets) | | cytotoxicity | |
| IL-22 | IL22R1 and | Th1 and Th17 | Fibroblasts, | Inflammatory, antimicrobial | ↑ = psoriasis |
| | IL10R2; | cells, NK | epithelial cells | | |
| | IL22BP | cells | | | |
| IL-23 | IL23R | Macrophages | T cells | Inflammatory; promotes | ↓ = susceptibility |
| (p19 + | andIL12Rb1 | and dendritic | | proliferation of Th17 cells | to extracellular |
| p40) | | cells | | | pathogens. ↑ = |
| | | | | | exacerbates |
| | | | | | organ- specific |
| | | | | | autoimmune |
| | | | | | inflammation. |
| IL-24 | IL20R1, | Monocytes, | Keratinocytes | | ↑ = antitumor |
| | IL22R1, | CD4+ T cells | | | effects |
| | IL20R2 | | | | |
| IL-25 | IL17RB | Th2 cells, | Non-B, non-T, | Promotes Th2 | ↓ = impairs Th2 |
| (IL-17E) | | mast cells | cKit+, FcεR− | differentiation and | responses to |
| | | | cells | proliferation | extracellular |
| | | | | | pathogens such |
| | | | | | as worms |
| IL-26 | IL22R1 and | Activated T | | | |
| | IL10R2 | cells | | | |
| IL-27 | WSX-1 and | Activated | T cells, others | Induction of early Th1 | ↓ = immune |
| (p28 + | gp130 | dendritic cells | | differentiation by | pathology due to |
| EBI3) | | | | stimulating expression of | uncontrolled |
| | | | | the Tbet transcription | inflammatory |
| | | | | factor; Inhibition of effector | response |
| | | | | Th17 cel responses by | |
| | | | | inducing STAT-1- | |
| | | | | dependent blockade of IL- | |
| | | | | 17 production | |
| IL-28A/B/ | IL28R1 and | Activated | | May promote antiviral | |
| IL29 | IL10R2 | subsets of | | responses | |
| (IFNλ | | dendritic | | | |
| family) | | cells? | | | |
| IL-30 | | | | | |
| (p28subunit | | | | | |
| of IL-27) | | | | | |
| IL-31 | IL31Rα and | Activated T | Myeloid | Proinflammatory | ↑ = atopic |
| | OSM-Rβ | cells | progenitors, lung | | dermatitis; |
| | | | epithelial cells, | | allergic asthma |
| | | | keratinocytes | | |
| IL-32 | | | | Induces proinflammatory | |
| | | | | cytokine production | |
| IL-33 | ST2 and | Macrophages, | Mast cells, Th2 | Costimulation, promotes | ↑ = atopic |
| | IL1R-AcP | dendritic cells | cells | Th2 cytokine production | dermatitis, |
| | | | | | allergic asthma |
| IL-35 | | Tregs | Effector T cells | Immune suppression | |
| (p35 + | | | | | |
| EBI3) | | | | | |

Tumor Necrosis Factor (TNF)

| | | | | Cytokine | |
|---|---|---|---|---|---|
| | Cytokine | Cytokine | Cytokine | Main | Cytokine Disease |
| Cytokine | Receptor | Source | Targets | Function | Association |
| TNF | Murine: | Macrophages, | Neutrophils, | Inflammatory; | ↓ = disregulated fever; |
| alpha | TNFR, p55; | monocytes, T | macrophages, | promotes | increased susceptibility |
| | TNFR, p75 | cells, others | monocytes, | activation | to bacterial infection; |
| | Human: | | endothelial cells | and | enhanced resistance to |
| | TNFR, p60; | | | production of | LPS-induced septic |
| | TNFR, p80 | | | acute-phase | shock ↑ = exacerbation |
| | | | | proteins | of arthritis and colitis |
| LT alpha | Murine: | T cells, B cells | Many cell types | Promotes | ↓ = defective response |
| | TNFR, p55; | | | activation | to bacterial pathogens; |
| | TNFR, p75 | | | and | absence of peripheral |
| | Human: | | | cytotoxicity; | lymph nodes and Peyer's |
| | TNFR, p60; | | | development | patches |
| | TNFR, p80 | | | of lymph | |
| | | | | nodes and | |
| | | | | Peyer's | |
| | | | | patches | |
| LT beta | LTbR | T cells, B cells | Myeloid cells, | Peripheral | ↓ = increased |
| | | | other cell types | lymph node | susceptibility to |
| | | | | development; | bacterial infection; |
| | | | | proinflammatory | absence of lymph nodes |
| | | | | | and Peyer's patches |
| | | | | | ↑ = ectopic lymph node |
| | | | | | formation |
| LIGHT | LTbR, DcR3, | Activated T | B cells, NK cells, | Costimulatory; | ↓ = defective CD8 T cell |
| | HVEM | cells, | DCs, other tissue | promotes | costimulation |
| | | monocytes, DCs | | CTL activity | |
| TWEAK | Fn14 | Monocytes, | Tissue | Proinflammatory; | |
| | | macrophages, | progenitors, | promotes | |
| | | endothelial | epithelial, | cell growth | |
| | | | endothelial | for tissue | |
| | | | | repair and | |
| | | | | remodeling | |
| APRIL | TACI, BAFF- | Macrophages, | B cell subsets | Promotes T | ↓ = impaired class |
| | R, BCMA | DCs | | cell- | switching to IgA |
| | | | | independent | |
| | | | | responses; B | |
| | | | | cell | |
| | | | | homeostasis | |
| | | | | and | |
| | | | | differentiation | |
| BAFF | TACI, BAFF- | Macrophages, | B cells | B cell | ↓ = B cell lymphopenia: |
| (BlvS) | R, BCMA | DCs, astrocytes | | maturation | defective humoral |
| | | | | and survival | immunity ↑ = SLE-like |
| | | | | | syndrome |
| TL1A | DcR3, DR3 | Macrophages, | Activated T cells | Promotes | GITRL |
| | | endothelial cells | | proliferation | |
| | | | | and cytokine | |
| | | | | production | |
| GITRL | GITR | DCs, | T regulatory | Costimulatory | |
| | | macrophages, B | cells, activated T | | |
| | | cells, others | cells | | |
| OX40L | OX40 | Activated T | T cells, B cells, | Costimulatory; | ↓ = impaired humoral |
| | | cells, B cells, | DCs | activation | responses |
| | | DCs, monocytes | | and migration | |
| | | | | of monocytes | |
| CD40L | CD40 | T cells, | B cells, APCs | Costimulatory; | ↓ = defective antibody |
| (CD154) | | monocytes, | | promotes T | responses and germinal |
| | | macrophages, | | cell- | center formation; hyper- |
| | | others | | dependent | IgM syndrome ↑ = SLE- |
| | | | | responses; B | like syndrome |
| | | | | cell | |
| | | | | differentiation | |
| | | | | and class | |
| | | | | switching | |
| FASL | FAS, DcR3 | Activated T | APCs, many | Regulatory; | ↓ = lymphoproliferative |
| | | cells, B cells, | other cell types | pro apoptotic | disease and systemic |
| | | and NK cells | | | autoimmunity |
| CD27L | CD27 | Activated T cels, | T cells, activated | Costimulatory | |
| (CD70) | | B cells, DCs, | B cells | | |
| | | monocytes | | | |
| CD30L | CD30 | Neutrophils, B | T cells, B cells | Costimulatory; | Viral CD30 blocks Th1 |
| (CD153) | | cells, | | promotes | response |
| | | macrophages, | | proliferation | |
| | | activated T cells | | and cytokine | |
| | | | | production | |
| 4-1BBL | 4-1BB | Activated T | Activated T cells, | Costimulatory; | |
| | | cells, B cells, | B cells, DCs | promotes | |
| | | DCs, monocytes, | | activation | |
| | | macrophages | | and migration | |
| | | | | of monocytes | |
| TRAIL | TRAIL-R1 | Activated NK | Many cell types | Costimulatory; | ↓ = defective NK- |
| | (DR4), | cells, T cells | | promotes | mediated antitumor |
| | R2(DR5), R3 | | | NK cell | response ↑ = enhanced |
| | (DcR1), and | | | functions; | responsiveness to |
| | R4(DcR2) | | | proapoptotic | autoantigens |
| RANK | RANK | T cells and | Osteoclasts, | Costimulatory; | ↓ = osteopetrosis ↑ = |
| Ligand(TRANCE) | receptor or | osteoblasts | many cell types | promotes | osteoporosis |
| | osteoprotegrin | | | osteoclastogenesis | |
| | | | | and | |
| | | | | cytokine | |
| | | | | production | |

TABLE 11 depicts interleukins and their respective cell interactions and phenotypic effects.

Other Cytokines

| | Cytokine | Cytokine | Cytokine | Cytokine Main | Cytokine Disease |
|---|---|---|---|---|---|
| Cytokine | Receptor | Source | Targets | Function | Association |
| FLT3 | Receptor | Diverse | DCs, other | Differentiation and | ↓ = impaired |
| Ligand | tyrosine | tissue | myeloid cells | proliferation; synergizes | hematopoietic stem |
| | kinases | | | with stem cell factor | cell repopulation |
| | | | | | and B cell |
| | | | | | precursors |
| G-CSF | GCSFRdimer | Macrophages, | Committed | Differentiation and | ↓ = neutropenia |
| | | fibroblasts, | progenitors | activation of | |
| | | other tissues | | granulocytes | |
| GM-CSF | GM-CSFRα, | T cells, | Macrophages, | Inflammatory; induction | ↓ = affects alveolar |
| | βc | macrophages, | granulocytes, | of activation; differentiation, | function |
| | | fibroblasts, | dendritic | growth, and | |
| | | others | cells, and | survival | |
| | | | progenitors | | |
| IFNα, IFNβ, | IFNαR1, | Macrophages, | NK cells, | Promotes resistance to | ↓ = impaired |
| IFNω | IFNαR2 | fibroblasts, | many others | viral pathogens; | antiviral responses |
| | | plasmacytoid | | promotes increased | |
| | | DCs, | | expression of MHC class | |
| | | others | | I | |
| IFNγ | IFNγR1, | Th1 cells, | Macrophages, | Promotes activation of | ↓ = susceptibility |
| | IFNγR2 | NK cells, | NK cells, T | APCs and cell-mediated | to intracellular |
| | | CD8 T cells | cells, others | immunity; increased | pathogens |
| | | | | MHC class II expression | |
| LIF | LIFR, gp130 | Macrophages, | Embryonic | Cell survival | ↓ = deficient |
| | | T cells, | stem cells, | | hematopoietic |
| | | fibroblasts, | hematopoietic | | progenitor cells; |
| | | uterus, | cells, others | | defective |
| | | others | | | blastocyst |
| | | | | | implantation |
| M-CSF | Receptor | Monocytes, | Committed | Differentiation; | ↓ = monocyte |
| | tyrosine | fibroblasts, | myeloid | proliferation and | deficiency; |
| | kinases | others | progenitors | survival | osteopetrosis |
| MIF | CD74trimer, | Macrophages, | Macrophages | Cell migration, DTH | ↓ = susceptibility |
| | CD44 | T cells | | response | to Gram-negative |
| | | | | | bacteria |
| OSM | LIFR or | Macrophages, | Myeloid cells, | Differentiation; | |
| | OSM- Rβ, | fibroblasts, | embryonic | induction of immune | |
| | gp130 | others | stem cells, T | response (early) | |
| | | | cells, others | | |
| Stem Cell | Receptor | Bone | Stem cells, | Activation and growth | ↓ = impaired |
| Factor | tyrosine | marrow | mast cells | | hematopoietic stem |
| (SCF) | kinases | | | | cell proliferation |
| | | | | | and melanocyte |
| | | | | | production |
| TGFβ1, | TGFβR type | T cells, | All leukocyte | Regulatory; inhibits | ↓ = increased |
| TGFβ2, | I, type II, and | DCs, | populations | growth and activation; | susceptibility to |
| TGFβ3 | type III | macrophages, | | Treg maintenance; | autoimmune |
| | | others | | synergizes with IL-6 to | disorders ↑ = |
| | | | | promote Th17 | fibrotic diseases |
| TSLPLigand | TSLPR, | Skin, lung, | DCs and other | Promotes Th2 development | ↑ = atopic diseases |
| | IL7Rα | and gut | myeloid cells | (human); B cell | |
| | | | | development (mouse) | |

TABLE 12 depicts additional cytokines and their respective cell interactions and phenotypic effects.
References:
1. SnapShot: Cytokines I Cristina M. Tato and Daniel J. Cell 132, p. 324
2. SnapShot: Cytokines II Cristina M. Tato and Daniel J. Cell 132, p. 500
3. SnapShot: Cytokines III Cristina M. Tato and Daniel J. Cell 132, p. 900
4. SnapShot: Cytokines IV Cristina M. Tato and Daniel J. Cell 132, p. 1062

| Systematic name | | |
|---|---|---|
| (common name) | Receptor | |
| CC chemokine/ | CCL1(I-309) | CCR8, R11 |
| receptor family | CCL2 (MCP-1, MCAF) | CCR2 |
| | CCL3 (MIP-1α/LD78α) | CCR1, R5 |
| | CCL3L1 (LD78β) | CCR5 |
| | CCL4 (MIP-1β) | CCR5 |
| | CCL4L1 | CCR5 |
| | CCL4L2 | CCR5 |
| | CCL5 (RANTES) | CCR1, R3, R4, R5 |
| | CCL6 (C-10) | CCR1, R2, R3 |
| | CCL7 (MCP-3) | CCR1, R2, R3 |
| | CCL8 (MCP-2) | CCR1, R2, R5, |
| | | R11 |
| | CCL9 (MRP-2/MIP-1γ) | CCR1 |
| | CCL10 (MRP-2/MIP-1γ) | CCR1 |
| | CCL11 (Eotaxin) | CCR3 |
| | CCL12 (MCP-5) | CCR2 |
| | CCL13 (MCP-4) | CCR1, R2, R3, |
| | | R11 |
| | CCL14 (HCC-1) | CCR1 |
| | CCL15 (HCC-2, Lkn-1) | CCR1, R3 |
| | CCL16 (HCC-4, LEC) | CCR1 |
| | CCL17 (TARC) | CCR4 |
| | CCL18 (DC-CK1, PARC) | Unknown |
| | CCL19 (MIP-3β, ELC) | CCR7, R11 |
| | CCL20 (MIP-3α, LARC) | CCR6 |
| | CCL21 (6Ckine, SLC) | CCR7, R11 |
| | CCL22 (MDC, STCP-1) | CCR4 |
| | CCL23 (MPIF-1) | CCR1 |
| | CCL24 (MPIF-2, Eotaxin-2) | CCR3 |
| | CCL25 (TECK) | CCR9, R11 |
| | CCL26 (Eotaxin-3) | CCR3 |
| | CCL27 (CTACK, ILC) | CCR2, R3, R10 |
| | CCL28 (MEC) | CCR3, R10 |
| C chemokine/ | XCL1 (Lymphotactin) | XCR1 |
| receptor family | XCL2 (SCM1-b) | XCR1 |
| CXC chemokine/ | CXCL1 (GROα, MGSA-α) | CXCR2 > R1 |
| receptor family | CXCL2 (GROβ, MGSAβ) | CXCR2 |
| | CXCL3 (GROγ, MGSAγ) | CXCR2 |
| | CXCL4 (PF4) | CXCR3 |
| | CXCL4L1 (PF4V1) | CRCR3 |
| | CXCL5 (ENA-78) | CXCR1, R2 |
| | CXCL6 (GCP-2) | CXCR1, R2 |
| | CXCL7 (NAP-2) | CXCR2 |
| | CXCL8 (IL-8) | CXCR1, R2 |
| | CXCL9 (Mig) | CXCR3 |
| | CXCL10 (IP-10) | CXCR3 |
| | CXCL11 (I-TAC) | CXCR3 |
| | CXCL12 (SDF-1α/β) | CXCR4, R7 |
| | CXCL13 (BLC, BCA-1) | CXCR3, R5 |
| | CXCL14 (BRAK, bolekine) | Unknown |
| | CXCL15 | Unknown |
| | CXCL16 (SR-PSOX) | CXCR6 |
| | CXCL17 (VCC1, DMC) | Unknown |
| CX3C chemokine/ | CX3CL1 (Fractalkine) | |
| receptor family | | |

Table 13 depicts additional chemokines and their respective cell receptors (https://www.sciencedirect.com/science/article/pii/S0167488914001967).

TABLE 14
depicts examples of secreted proteins of interest that could be delivered
to cells such as cancer cells (e.g., using a ligand-targeted
nanoparticle) to influence the cell or cancer's microenvironment.

| Protein | Expression Type | Action | Sequence | SEQ ID NO |
|---|---|---|---|---|
| IL-2 | Secreted cytokine | Tumor | MYRMQLLSCIALSLA | |
| | | microenvironment | LVTNSAPTSSSTKKTQ | |
| | | modulation | LQLEHLLLDLQMILNG | |
| | | | INNYKNPKLTRMLTF | |
| | | | KFYMPKKATELKHLQ | |
| | | | CLEEELKPLEEVLNLA | |
| | | | QSKNFHLRPRDLISNI | |
| | | | NVIVLELKGSETTFMC | |
| | | | EYADETATIVEFLNR | |
| | | | WITFCQSIISTLT | |
| IL-7 | Secreted cytokine | Tumor | MFHVSFRYIFGLPPLIL | |
| | | microenvironment | VLLPVASSDCDIEGKD | |
| | | modulation | GKQYESVLMVSIDQL | |
| | | | LDSMKEIGSNCLNNEF | |
| | | | NFFKRHICDANKEGM | |
| | | | FLFRAARKLRQFLKM | |
| | | | NSTGDFDLHLLKVSE | |
| | | | GTTILLNCTGQVKGR | |
| | | | KPAALGEAQPTKSLEE | |
| | | | NKSLKEQKKLNDLCF | |
| | | | LKRLLQEIKTCWNKIL | |
| | | | MGTKEH | |
| IL-12 | Secreted cytokine | Tumor | MCPARSLLLVATLVL | |
| | | microenvironment | LDHLSLARNLPVATPD | |
| | | modulation | PGMFPCLHHSQNLLR | |
| | | | AVSNMLQKARQTLEF | |
| | | | YPCTSEEIDHEDITKD | |
| | | | KTSTVEACLPLELTKN | |
| | | | ESCLNSRETSFITNGSC | |
| | | | LASRKTSFMMALCLS | |
| | | | SIYEDLKMYQVEFKT | |
| | | | MNAKLLMDPKRQIFL | |
| | | | DQNMLAVIDELMQAL | |
| | | | NFNSETVPQKSSLEEP | |
| | | | DFYKTKIKLCILLHAF | |
| | | | RIRAVTIDRVMSYLNAS | |
| IL-15 | Secreted cytokine | Tumor | MRISKPHLRSISIQCYL | |
| | | microenvironment | CLLLNSHFLTEAGIHV | |
| | | modulation | FILGCFSAGLPKTEAN | |
| | | | WVNVISDLKKIEDLIQ | |
| | | | SMHIDATLYTESDVHP | |
| | | | SCKVTAMKCFLLELQ | |
| | | | VISLESGDASIHDTVE | |
| | | | NLIILANNSLSSNGNV | |
| | | | TESGCKECEELEEKNI | |
| | | | KEFLQSFVHIVQMFIN | |
| | | | TS | |
| IL-21 | Secreted cytokine | Tumor | MERIVICLMVIFLGTL | |
| | | microenvironment | VHKSSSQGQDRHMIR | |
| | | modulation | MRQLIDIVDQLKNYV | |
| | | | NDLVPEFLPAPEDVET | |
| | | | NCEWSAFSCFQKAQL | |
| | | | KSANTGNNERIINVSI | |
| | | | KKLKRKPPSTNAGRR | |
| | | | QKHRLTCPSCDSYEK | |
| | | | KPPKEFLERFKSLLQK | |
| | | | MIHQHLSSRTHGSEDS | |
| IFN-gamma | Secreted cytokine | Tumor | MKYTSYILAFQLCIVL | |
| | | microenvironment | GSLGCYCQDPYVKEA | |
| | | modulation | ENLKKYFNAGHSDVA | |
| | | | DNGTLFLGILKNWKE | |
| | | | ESDRKIMQSQIVSFYF | |
| | | | KLFKNFKDDQSIQKSV | |
| | | | ETIKEDMNVKFFNSN | |
| | | | KKKRDDFEKLTNYSV | |
| | | | TDLNVQRKAIHELIQV | |
| | | | MAELSPAAKTGKRKR | |
| | | | SQMLFRGRRASQ | |

Payloads that lead to cancer cell cytotoxicity (including any variants thereof that retain their cytotoxic function)

TABLE 15
depicts examples of proteins of interest that could be delivered to cancer cells
(e.g., using a subject nanoparticle with an appropriate targeting ligand).

| | | | | SEQ ID | |
|---|---|---|---|---|---|
| Protein | Expression Type | Action | Sequence | NO | Notes |
| GM-CSF | Secreted cytokine | Tumor | MWLQNLLLLGAVVCS | | Talimogene |
| | | microenvironment | ISAPTRLPSPVTRPWQ | | laherparepvec |
| | | modulation | HVDAIKEALSLLNNSN | | (T-VEC, |
| | | | DTAAVMNETVDVVC | | Imlygic ™), a |
| | | | KMFDPQEPTCVQTRL | | genetically |
| | | | NLYKQGLRGSLTRLK | | modified herpes |
| | | | SPLTLLAKHYEQHCPL | | simplex virus |
| | | | TEETSCETQSITFKSFK | | expressing GM- |
| | | | DSLNKFLFTIPFDCWG | | CSF recently |
| | | | PVKK | | licensed for the |
| | | | | | treatment of |
| | | | | | melanoma |
| apoptin | Protein | Apoptosis inducer | MQTPRSRRRATTTQSE | | |
| | | | LLTAYEHPTSSSPPAE | | |
| | | | TTSIEIQIGIGSTIITLSL | | |
| | | | PGYASVRVLTTRSAPA | | |
| | | | DDGGVTGSRRLVDLS | | |
| | | | HRRPRRTSSPEIYVGF | | |
| | | | AAKEKQQKENLITLRE | | |
| | | | NGPPIKKLRL | | |
| lactaptin | Protein | Apoptosis inducer | MKSFLLVVNALALTL | | Lactaptin is a |
| | | | PFLAVEVQNQKQPAC | | fragment of |
| | | | HENDERPFYQKTAPY | | human milk |
| | | | VPMYYVPNSYPYYGT | | kappa-casein |
| | | | NLYQRRPAIAINNPYV | | (residues 57-134) |
| | | | PRTYYANPAVVRPHA | | |
| | | | QIPQRQYLPNSHPPTV | | |
| | | | VRRPNLHPSFIAIPPKK | | |
| | | | IQDKIIIPTINTIATVEP | | |
| | | | TPAPATEPTVDSVVTP | | |
| | | | EAFSESIITSTPETTTV | | |
| | | | AVTPPTA | | |

### IV. Affinity Markers

In some embodiments, a delivery vehicle (e.g., a nanoparticle such as a targeted nanoparticle) is used to influence protein expression and/or cell surface composition of a target cell such as a cancerous tissue thereby bolstering the adaptive immune response and overcoming physiological hurdles faced in the treatment of solid tumors. Thus, in some embodiments delivery of a payload results in expression and presentation of a protein of interest (e.g., an affinity marker) on the surface of the cell.

In some cases the affinity marker is a protein presented on the cell surface that is highly immunogenic and is a “non-self” domain. This approach can bypass the central tolerance in the thymus. Delivery using non-viral delivery vehicles such as nanoparticles mitigates barriers faced by viral delivery because nanoparticles do not express immunogenic epitopes on their surface and are stealth from the immune system until interaction with the targeted cancer cells.

As such, in some cases a payload is an affinity marker (or a nucleic acid encoding same). The term “affinity marker” is used herein to refer to a polypeptide presented on the cell surface (e.g., via forced heterologous expression in a target cell such as a cancer cell) that may elicit an endogenous adaptive immune response (against the affinity marker) and/or may act as a target for T-Cell therapy. In some cases an affinity marker is a naturally existing membrane protein, and in some cases an affinity marker is a chimeric polypeptide in which a membrane anchored region (e.g., a transmembrane domain) is fused to an extracellular portion that elicits an endogenous immune response or is targeted with T-cells that are engineered to recognize the affinity marker.

Thus, in some cases cancerous tissue can be “programmed” to present a distinct surface marker as a domain that is subsequently targeted by immune cells, triggering an adaptive immune response across many tumor subclonal populations. This approach presents an improvement to TCR or CAR engineering, and other single-marker targeted immuno-oncology approaches, in that the affinity marker (in some cases delivered via nanoparticle) induces a tumor-wide expression of adaptive immune learning cues. For particularly complex cancers with a diversity of clonal subpopulations, this leads to a more robust learning response and improved treatment. Additionally, the in vivo utility of this approach limits the need for complex and cumbersome autologous and allogeneic cell transplantation procedures.

In some cases cancerous tissue is programmed to present a distinct antigen as a functional domain that is subsequently targeted by an engineered (e.g., cytotoxic) T cell. The T Cell can possess a TCR or CAR that is specific to the antigen, and may be engineered ex vivo or in vivo.

An affinity marker payload can be delivered using any delivery vehicle. In some cases the delivery vehicle is a subject nanoparticle (e.g., a nanoparticle that includes a targeting ligand and/or a core comprising an anionic polymer composition, a cationic polymer composition, and a cationic polypeptide composition). In some cases the affinity marker is delivered using a delivery vehicle with a targeting ligand and in some cases using a delivery vehicle without a targeting ligand (e.g., the delivery vehicle can be delivered using local administration such as intratumoral injection).

An affinity marker payload can be delivered using personalized delivery (descried in more detail elsewhere herein)—meaning, e.g., that it can be delivered using a delivery vehicle designed using information from the individual/patient. For example, in some cases an affinity marker payload is delivered using a delivery vehicle with a targeting ligand and/or a promoter that was selected based on an individual's/patient's diagnostic evaluation. In some cases a subject affinity marker is a diagnostically responsive surface protein—meaning that the surface protein was determined to be enriched on the surface of cancer cells of an individual/patient or even specifically expressed by such cells.

In some cases, the affinity marker can stimulate innate immune activity (i.e., the affinity marker can be recognized by endogenous immune cells as signal of non-self, and this can trigger an endogenous immune system response against cells expressing that beacon). In some cases T-cells engineered to target the affinity marker can be co-administered (either in series or in parallel) with the delivery vehicle. The affinity marker may be any protein or protein fragment with a known protein-protein interaction, including endogenous human proteins, viral proteins, and synthetic de novo proteins. In some cases, an affinity marker engages a direct signaling cascade (for example, but not limited to—with a CAR-T/TCR).

TABLE 16
depicts exemplary non-limiting examples of affinity markers.

| | | | | SEQ ID | |
|---|---|---|---|---|---|
| Protein | Expression Type | Action | Sequence | NO | Notes |
| Adenovirus | Integral membrane | Transfect into | MTGSTIAPTTDYRNTT | | |
| death | glycoprotein that | tumor cells to elicit | ATGLTSALNLPQVHA | | |
| protein | localizes to the | immune response | FVNDWASLD | | |
| | inner and outer | (e.g., against both | | | |
| | nuclear membrane | the ADP and Tumor | | | |
| | and Golgi | antigens) | | | |
| | apparatus | | | | |
| modified | Surface | Transfect into | H2N- | | T represents an |
| Apa protein | glycoprotein of | tumor cells to elicit | DPEPAPPVPTTAASPPS | | O-glycosylated |
| | tuberculosis | immune response | TAAAPPAPATPVAPPP | | threonine |
| | | (e.g., against both | PAAANT-CONH2 | | functionalized |
| | | the ADP and Tumor | | | with 2 or 3 |
| | | antigens) | | | glycosidic |
| | | | | | residues, and Ac |
| | | | | | represents an |
| | | | | | acetate function |
| Claudin 6 | Integral membrane | a component of | MASAGMQILGVVLTL | | T represents an |
| (CLND6) | that is virtually | tight junction | LGWVNGLVSCALPM | | O-glycosylated |
| | absent from any | strands, which is a | WKVTAFIGNSIVVAQ | | threonine |
| | normal tissue but | member of the | VVWEGLWMSCVVQS | | functionalized |
| | aberrantly and | claudin family. The | TGQMQCKVYDSLLAL | | with 2 or 3 |
| | frequently | protein is an protein | PQDLQAARALCVIALL | | glycosidic |
| | expressed in | and is one of the | VALFGLLVYLAGAKC | | residues, and Ac |
| | ovarian, lung, | entry cofactors for | TTCVEEKDSKARLVL | | represents an |
| | gastric breast, | hepatitis C virus | TSGIVFVISGVLTLIPV | | acetate function |
| | prostate, and | | CWTAHAIIRDFYNPLV | | |
| | pediatric cancers | | AEAQKRELGASLYLG | | |
| | | | WAASGLLLLGGGLLC | | |
| | | | CTCPSGGSQGPSHYM | | |
| | | | ARYSTSAPAISRGPSE | | |
| | | | YPTKNYV | | |

In some cases, an affinity marker is a synthetic chimeric protein that includes a membrane anchor fused (e.g., via a linker—various linkers are described elsewhere herein and can be used in an affinity marker) to a functional domain that is displayed extracellularly by the cell that expresses it. Tables 17 and 18 provide examples of membrane anchors and extracellular polypeptides that can be used as part of an affinity marker. These “anchors” may represent conserved transmembrane domains of extracellularly-presenting affinity marker sequences, or sequence alignments for machine learning approaches for determining optimal ligand-receptor docking for a given cell/tissue/organ with one of these classes of proteins or homologues enriched. Rather than de novo modeling of ligand-receptor interactions, this approach allows for rapid design and synthesis of a targeting ligand or library of targeting ligands (e.g. selectively mutated amino acid residues and/or peptoid and/or synthetic amino acid and/or alternative polymer/glycoprotein modifications upon a native peptide or glycoprotein sequence). De novo modeling and synthesis approaches may also be used, either as part of selected mutagenesis libraries or alternative means of combinatorial/library prep. (e.g. SELEX, phage display, and similar techniques). These techniques are further enhanced by a modular nanoparticle, nanomaterials and gene editing/gene delivery platform approach for efficiently delivering these synthetic markers (e.g. affinity markers, transmembrane anchor domains detailed elsewhere) to specified cells/tissues/organs/cancers.

TABLE 17
depicts examples of membrane anchor classes for affinity markers
(including any variants thereof that retain their membrane
anchoring/embedding function).

| Protein Domain | Sequence | SEQ ID NO |
|---|---|---|
| Amino Acid Permease Signature | MSNTSSYEKNNPDNLKHNGITIDSEFLTQEPIT | |
| | IPSNGSAVSIDETGSGSKWQDFKDSFKRVKPI | |
| | EVDPNLSEAEKVAIITAQTPLKHHLKNRHLQ | |
| | MIAIGGAIGTGLLVGSGTALRTGGPASLLIGW | |
| | GSTGTMIYAMVMALGELAVIFPISGGFTT | |
| | YATRFIDESFGYANNFNYMLQWLVVLPL | |
| | EIVSASITVNFWGTDPKYRDGFVALFWL | |
| | AIVIINMFGVKGYGEAEFVFSFIKVITVVG | |
| | FIILGIILNCGGGPTGGYIGGKYWHDPGAF | |
| | AGDTPGAKFKGVCSVFVTAAFSFAGSEL | |
| | VGLAASESVEPRKSVPKAAKQVFWRITLF | |
| | YILSLLMIGLLVPYNDKSLIGASSVDAAA | |
| | SPFVIAIKTHGIKGLPSVVNVVILIAVLSV | |
| | GNSAIYACSRTMVALAEQRFLPEIFSYVD | |
| | RKGRPLVGIAVTSAFGLIAFVAASKKEGE | |
| | VFNWLLALSGLSSLFTWGGICICHIRFRK | |
| | ALAAQGRGLDELSFKSPTGVWGSYWGLF | |
| | MVIIMFIAQFYVAVFPVGDSPSAEGFFEA | |
| | YLSFPLVMVMYIGHKIYKRNWKLFIPAE | |
| | KMDIDTGRREVDLDLLKQEIAEEKAIMA | |
| | TKPRWYRIWNFWC | |
| EGF-Like Domain Signature | MRLLRRWAFAALLLSLLPTPGLGTQGPAGAL | |
| | RWGGLPQLGGPGAPEVTEPSRLVRESSGGEV | |
| | RKQQLDTRVRQEPPGGPPVHLAQVSFVIPAF | |
| | NSNFTLDLELNHHLLSSQYVERHFSREGTTQ | |
| | HSTGAGDHCYYQGKLRGNPHSFAALSTCQG | |
| | LHGVFSDGNLTYIVEPQEVAGPWGAPQGPLP | |
| | HLIYRTPLLPDPLGCREPGCLFAVPAQSAPPN | |
| | RPRLRRKRQVRRGHPTVHSETKYVELIVIND | |
| | HQLFEQMRQSVVLTSNFAKSVVNLADVIY | |
| | KEQLNTRIVLVAMETWADGDKIQVQDD | |
| | LLETLARLMVYRREGLPEPSDATHLFSGR | |
| | TFQSTSSGAAYVGGICSLSHGGGVNEYG | |
| | NMGAMAVTLAQTLGQNLGMMWNKHRS | |
| | SAGDCKCPDIWLGCIMEDTGFYLPRKFSR | |
| | CSIDEYNQFLQEGGGSCLFNKPLKLLDPP | |
| | ECGNGFVEAGEECDCGSVQECSRAGGNC | |
| | CKKCTLTHDAMCSDGLCCRRCKYEPRG | |
| | VSCREAVNECDIAETCTGDSSQCPPNLHK | |
| | LDGYYCDHEQGRCYGGRCKTRDRQCQV | |
| | LWGHAAADRFCYEKLNVEGTERGSCGR | |
| | KGSGWVQCSKQDVLCGFLLCVNISGAPR | |
| | LGDLVGDISSVTFYHQGKELDCRGGHVQ | |
| | LADGSDLSYVEDGTACGPNMLCLDHRCL | |
| | PASAFNFSTCPGSGERRICSHHGVCSNEG | |
| | KCICQPDWTGKDCSIHNPLPTSPPTGETER | |
| | YKGPSGTNIIIGSIAGAVLVAAIVLGGTG | |
| | WGFKNIRRGRSGGA | |
| GPS Domain Profile | MAPPAARLALLSAAALTLAARPAPSPGLGPE | |
| | CFTANGADYRGTQNWTALQGGKPCLFWNET | |
| | FQHPYNTLKYPNGEGGLGEHNYCRNPDGDV | |
| | SPWCYVAEHEDGVYWKYCEIPACQMPGNLG | |
| | CYKDHGNPPPLTGTSKTSNKLTIQTCISFCRS | |
| | QRFKFAGMESGYACFCGNNPDYWKYGEAAS | |
| | TECNSVCFGDHTQPCGGDGRIILFDTLVGAC | |
| | GGNYSAMSSVVYSPDFPDTYATGRVCYWTI | |
| | RVPGASHIHFSFPLFDIRDSADMVELLDG | |
| | YTHRVLARFHGRSRPPLSFNVSLDFVILY | |
| | FFSDRINQAQGFAVLYQAVKEELPQERPA | |
| | VNQTVAEVITEQANLSVSAARSSKVLYVI | |
| | TTSPSHPPQTVPGSNSWAPPMGAGSHRV | |
| | EGWTVYGLATLLILTVTAIVAKILLHVTF | |
| | KSHRVPASGDLRDCHQPGTSGEIWSIFYK | |
| | PSTSISIFKKKLKGQSQQDDRNPLVSD | |
| HIG1 Domain Profile | MSTDTGVSLPSYEEDQGSKLIRKAKEAPFVP | |
| | VGIAGFAAIVAYGLYKLKSRGNTKMSIHL\IH | |
| | MRVAAQGFVVGAMTVGMGYSMYREFWAK | |
| | PKP | |
| ITAM Motif Profile | MEHSTFLSGLVLATLLSQVSPFKIPIEELEDRV | |
| | FVNCNTSITWVEGTVGTLLSDITRLDLGKRIL | |
| | DPRGIYRCNGTDIYKDKESTVQVHYRMCQSC | |
| | VELDPATVAGIIVTDVIATLLLALGVFCFAGH | |
| | ETGRLSGAADTQALLRNDQVYQPLRDRDDA | |
| | QYSHLGGNWARNK | |

Examples of extracellular domains that can be used as part of an affinity marker domains are detailed through the sets of ligands and receptors outlined within this disclosure. In other words, a non-limiting example includes any ligand or receptor pairing outlined herein (or otherwise determined through proteomics and/or transcriptomics of a given cell population—or otherwise identifiable cell-specific markers) can be utilized to create an affinity marker. Many such pairings are detailed herein.

In some examples, an already-overexpressed protein may be further hyper-expressed within a target cell/tissue/organ/cancer type. For example, a transmembrane domain that is uniquely and/or differentially expressed within a target tumor (e.g. a transmembrane domain with high cell/tissue/organ specificity indices) may be used as a sequence that further includes an extracellular affinity domain (as detailed elsewhere) or a signaling domain (as with introduction of a GPCR, DREADD, or chimeric receptor). These extracellular domains may serve as affinity domains for chimerically-modified immune cells (or other cells, such as stem cells), and may be coupled to enhanced or suppressed immune/stem cell/other circulatory cell homing (e.g. chemotaxis) or signaling (e g enhanced killing response of a CD8+ T cell subpopulation, NK cell subpopulation; enhanced affinity of an antigen-presenting cell subpopulation).

These affinity domains may include any variants thereof that maintain their immune-stimulating function, as well as a multitude of immunogenic markers such as viral protein fragments and patient-defined preexisting immunity/allergy/immune-response-generating peptide/glycopeptide/lipopeptide/glycolipid sequences. A cancer neoantigen may also serve as an extracellular domain. Engagement of dendritic cells and other antigen-presenting cells (APCs, including gamma delta (γδ) T cells, as part of this platform is further detailed within this disclosure as a method and use for personalized immunotherapies. These personalized immunotherapies are designed to be in vivo, ex vivo, or through a combination of ex vivo and in vivo approaches, whereby a subject nanoparticle or delivery vehicle is administered with affinity for a patient's cancer or a specific subtype of cells that require secondary beaconing by an alternative cell subpopulation (e.g. senescent cells being targeted to generate affinity for an extracellularly presenting domain of an engineered stem cell engraftment. Other methods for regenerative therapies can be envisaged. An optional, secondary subject nanoparticle or delivery vehicle may be utilized to introduce a “standardized docking domain” into a specific immune subpopulation or combination of immune subpopulations, or alternatively to a specific “interactive cell population” whereby the interactive cell population is intended to have a signaling and/or chemotactic effect with its local environment and the secondarily targeted set of cells.

Advances in rapid DNA synthesis technology further facilitate these innovations, whereby cancer-diagnostic determined (e.g. diagnostically-responsive transcriptomic and cell surface proteomic) transmembrane sequences may be introduced into a patient (following DNA synthesis or mRNA amplification/synthesis of the appropriate sequence) as part of a nanoparticle-administered immunotherapy, whereby the transmembrane domain (“cell/tissue/organ/cancer personalized transmembrane domain”) serves as a further anchor for an affinity domain (a ligand or receptor or fragment thereof as outlined elsewhere in this disclosure) and is encoded by the delivered DNA. Numerous library-generation DNA approaches may be utilized to combinatorially screen top-performing nanoparticle candidates delivering a variety of transgenes to a cell, tissue, organ or cancer type, and evaluate directed mutagenic libraries. For example, a large TCR mutagenic library may be utilized and transfected into T cells to establish optimal cancer-killing effects of a given recognition and signaling domain. Gene editing approaches and gene insertion approaches may be utilized as well, whereby donor DNA templates are customized for each patient and can be combinatorially or singly evaluated for their 1) gene insertion efficiency and/or 2) phenotypic effect. Rapid DNA synthesis may be coupled to existing peptide, polymer and/or ligand/anchor/linker libraries and is further supported by rapid peptide synthesis and predictive ligand-receptor modeling with optional high-throughput fluid-handling robotic workflows in the case of nanoparticle synthesis or library preparation with a variety of drug/RNA/DNA/protein-ligand conjugation techniques. Top-performing nanomedicine candidates can readily be applied to microfluidic and millifluidic scale-up techniques as well as parallel arrays of microfluidic devices for milligram-to-kilogram scale synthesis. Newly synthesized (e.g. high-throughput synthesized) peptide sequences may be coupled to anchor-linker or anchor libraries (detailed elsewhere) through numerous means further facilitated by flow-based synthesis and fluid-handling techniques. These peptide or ligand-polymer sequences may be combinatorially assembled with a variety of genetic, protein or small molecule payloads, as well as directly chemically conjugated to numerous surfaces and reactive domains, to enable multimodal and “super-personalized” diagnostically-responsive therapies. The ligands used herein and their associated anchors and linkers may also be introduced to recombinant protein sequences (e.g. recombinant Cas9-ligand, recombinant TALEN-ligand, recombinant recombinase-ligand) or modified nucleic acids/PNAs/MNAs/LNAs (e.g. modRNA-ligand, PNA-ligand, DNA-PNA-ligand, RNA-DNA-ligand, and the like) either homovalently or heterovalently through the methods and uses described herein (the “diagnostically-responsive” workflows. Combinatorial genes with DNA/RNA/PNA/LNA barcodes may also be used to create large pooled libraries of nanoparticles that can be subsequently sequenced in target cells, allowing for each formulation to have its own tag for subsequent identification in cell, organ-on-chip or animal models.

As noted above, in some cases, introduction of a payload encoding/carrying an affinity marker into a target cell results in the expression of the affinity marker on the surface of a targeted cell such as a cancer cell. In some such cases, this is coupled with a T-cell therapy in which T cells are engineered to recognize the affinity marker. The T cells can be introduced into the individual as part of a T cell therapy (after being engineer in vitro/ex vivo to express the desired receptor), or the T cells can be engineered endogenously (edited in vivo) in the individual. To accomplish the engineering, the T cell receptor (TCR) locus (e.g., alpha, beta, delta, and/or gamma subunit) of T cells can be edited so that the T cells express an engineered receptor that can specifically bind to the desired affinity marker. T cells can also be engineered to express a chimeric antigen receptor (CAR). Either way, the engineered T cells specifically recognize and target those cells that were targeted to express the affinity marker.

As one example, a NY-ESO antigen sequence may be inserted into cancer cells, and a corresponding NY-ESO-targeted TCR may be used with gamma delta (γδ) T cells in order to create an enhanced antigen-presenting effect following T cell distribution within the target cancer. Other antigen-presenting cells or αβ T cells may also be utilized.

In some cases, an affinity marker can be used to aid cell engraftment (e.g., stem cell engraftment when administering stem cells to a patient). Thus, in some cases, an affinity includes a functional domain that grants a cell affinity to a tissue, organ, or tissue environment of interest (e.g., when the affinity marker is expressed on the cell's surface). This is of particular interested for use in regenerative medicine applications where this may promote proper engraftment of cells in the desired environment and in the desired phenotype. For example, expanded stem cells can lose their phenotypic surface presentation and can be unable to migrate and/or engraft properly. They can also become trapped in the liver, lung, and/or spleen. Because of this, sometimes as little as 1% can reach the target tissue/disease area. In addition, direct injection of cells at the target organ can include a risk of hemorrhage and other complications associated with the administration method. Cell survival is also a shortcoming. To the contrary, affinity markers can promote adhesion to proper tissue compartment so that proper engraftment is achieved, as well as promote migration from the site of administration to the target organ thereby mitigating problems associated with expansion of both autologous and allogeneic stem cells. Thus, in some cases, affinity markers are expressed on stem cells that can be used in adoptive cell transfer. The stem cells can be any stem cell (e.g., endoderm, ectoderm, mesoderm stem cells; hematopoietic stem cells; mesenchymal stem cells; neural stem cells; endocrine precursors; and the like). When using stem cells for such applications, the stem cells can in some cases differentiate into any desired cell/tissue type (e.g., cartilage, bone, cardiomyocytes, neurons, adipocytes, osteoblasts, hepatocytes, myoblasts, neuron-like cells, and the like). The target organs/tissues can include, e.g., kidney, AKI administered for tubular endothelial cell repair, inflamed bowel, lung, bone, bone marrow, ischemic tissue, myocardial infarct damaged tissue, wounds, and the like. Such applications can be used for, e.g., diabetes, beta cell pathologies, myocardial infarction, brain trauma, and multiple sclerosis. Examples can include, e.g., migratory receptors of the CXC, CC, XC, CX3C families (e.g., CCR1, CCR2, CCR7, CXCR4/SDF-1, CX3CR1, CXCR6, c-met, CD44), which respond to proteins such as CXCL9, CXCL16, CCL20, CCL25, HGF, MCP-3, CXCL12, and HIF. In some case, e.g., when using hematopoietic stem cells, example proteins can include CCR1, CCR4, CCR7, CXCR5, and CCR10. In some cases stem cells can be used for their immunomodulatory abilities due to their ability to secrete a wide variety of growth factors and cytokines, with a subset that may have a profound effect on modulating immune response.

### Delivery

In some cases a subject method includes using a delivery vehicle to deliver a payload to a target cell, e.g., via administration to an individual, via transfection, via a nanoparticle, via a delivery molecule, etc. In some cases two or more different payloads are introduced into the cell as part of the same delivery vehicle (e.g., nanoparticle, delivery molecule, etc.). The payload can be delivered to any desired target cell, e.g., any desired eukaryotic cell such as a cancer cell.

In some cases the target cell is in vitro (e.g., the cell is in culture), e.g., the cell can be a cell of an established tissue culture cell line. In some cases the target cell is ex vivo (e.g., the cell is a primary cell (or a recent descendant) isolated from an individual, e.g. a patient). In some cases, the target cell is in vivo and is therefore inside of (part of) an organism.

A delivery vehicle may be introduced to a subject (i.e., administered to an individual) via any of the following routes: systemic, local, parenteral, subcutaneous (s.c.), intravenous (i.v.), intracranial (i.c.), intraspinal, intraocular, intradermal (i.d.), intramuscular (i.m.), intralymphatic (id.), or into spinal fluid. The components may be introduced by injection (e.g., systemic injection, direct local injection, local injection into or near a tumor and/or a site of tumor resection, etc.), catheter, or the like. Examples of methods for local delivery (e.g., delivery to a tumor and/or cancer site) include, e.g., by bolus injection, e.g. by a syringe, e.g. into a joint, tumor, or organ, or near a joint, tumor, or organ; e.g., by continuous infusion, e.g. by cannulation, e.g. with convection (see e.g. US Application No. 20070254842, incorporated here by reference).

The number of administrations of treatment to a subject may vary. Introducing a delivery vehicle into an individual may be a one-time event; but in certain situations, such treatment may elicit improvement for a limited period of time and require an on-going series of repeated treatments. In other situations, multiple administrations of a delivery vehicle may be required before an effect is observed. As will be readily understood by one of ordinary skill in the art, the exact protocols depend upon the disease or condition, the stage of the disease and parameters of the individual being treated.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of a payload is an amount that is sufficient, when administered to the individual, to palliate, ameliorate, stabilize, reverse, prevent, slow or delay the progression of a disease state/ailment.

In some cases, the target cell is a mammalian cell (e.g., a rodent cell, a mouse cell, a rat cell, an ungulate cell, a cow cell, a sheep cell, a pig cell, a horse cell, a camel cell, a rabbit cell, a canine (dog) cell, a feline (cat) cell, a primate cell, a non-human primate cell, a human cell). Any cell type can be targeted, and in some cases specific targeting of particular cells depends on the presence of targeting ligands (e.g., as part of a surface coat of a nanoparticle, as part of a delivery molecule, etc), where the targeting ligands provide for targeting binding to a particular cell type. For example, cells that can be targeted include but are not limited to bone marrow cells, hematopoietic stem cells (HSCs), long-term HSCs, short-term HSCs, hematopoietic stem and progenitor cells (HSPCs), peripheral blood mononuclear cells (PBMCs), myeloid progenitor cells, lymphoid progenitor cells, T-cells, B-cells (e.g., via targeting CD19, CD20, CD22), NKT cells, NK cells, dendritic cells, monocytes, granulocytes, erythrocytes, megakaryocytes, mast cells, basophils, eosinophils, neutrophils, macrophages (e.g., via targeting CD47 via SIRPα-mimetic peptides), erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, muscle cells, skeletal muscle cells, cardiac muscle cells, hepatic cells, fat cells, intestinal cells, cells of the colon, and cells of the stomach.

Examples of various applications (e.g., for targeting neurons, cells of the pancreas, hematopoietic stem cells and multipotent progenitors, etc.) are discussed above, e.g., in the context of targeting ligands. For example, hematopoietic stem cells and multipotent progenitors can be targeted for gene editing (e.g., insertion) in vivo. Even editing 1% of bone marrow cells in vivo (approximately 15 billion cells) would target more cells than an ex vivo therapy (approximately 10 billion cells) and in many cases (such as with sickle cell disease) the pathology will innately positively select for a cell chimerism (e.g. the targeted and edited cell populations expanding preferentially due to survival-enhancing pleiotropic effects of HBB edits). In vivo applications are amenable to repeat dosing with a non-viral platform consisting of native human protein fragments and other targeting ligand/constituent polymer designs that are unlikely to be immunogenic, and can particularly benefit from techniques for selective expansion either through direct programming e.g. a stem cell differentiation factor, or pleiotropic effects as outlined above). As another example, pancreatic cells (e.g., (3 islet cells) can be targeted, e.g., to treat pancreatic cancer, to treat diabetes, etc. In an exemplary embodiment, pancreatic B islets in Type I diabetes, if engineered to be less prone to autoimmunity, would also innately experience positive selection vs. non-targeted cells following treatment similarly to HSCs edited to be free of the sickle cell trait. As another example, somatic cells in the brain such as neurons can be targeted (e.g., to treat indications such as Huntington's disease, Parkinson's (e.g., LRRK2 mutations), and ALS (e.g., SOD1 mutations) and may experience enhanced survival or stem cell renewal following treatment). Additionally, targeted cells may have multiple genetic, protein, or small molecule instructions delivered to them, whereby edited or modified cells will experience asymmetrical cell division (e.g. enhanced cell division) in response to growth-stimulatory or cell differentiation cues (e.g. IL2 mRNA or mRNA/DNA/molecules encoding a cytokine/chemokine activity in immune cells; SCF, NGF, or other growth factor/Yamanaka factor mRNA or mRNA/DNA/molecules encoding a cell differentiation cue in stem cell poopulations, etc.). In some cases neural targeting can be achieved through direct intracranial injections. In other cases treatment of a cancer may be presented following resection of a tumor, to cause local environmental programming. Other local injection approaches may be utilized with or without ligand targeting in order to provide local effects and optional multimodal programming (e.g. gene edit+mRNA, gene edit+small molecules, mRNA+DNA, and the like).

As another example, endothelial cells and cells of the hematopoietic system (e.g., megakaryocytes and/or any progenitor cell upstream of a megakaryocyte such as a megakaryocyte-erythroid progenitor cell (MEP), a common myeloid progenitor cell (CMP), a multipotent progenitor cell (MPP), a hematopoietic stem cells (HSC), a short term HSC (ST-HSC), an IT-HSC, a long term HSC (LT-HSC)—see, e.g., FIGS. 6A-B) can be targeted with a subject nanoparticle (or subject viral or non-viral delivery vehicle) to treat Von Willebrand's disease. For example, a cell (e.g., an endothelial cell, a megakaryocyte and/or any progenitor cell upstream of a megakaryocyte such as an MEP, a CMP, an MPP, an HSC such as an ST-HSC, an IT-HSC, and/or an LT-HSC) harboring a mutation in the gene encoding von Willebrand factor (VWF) can be targeted (in vitro, ex vivo, in vivo) in order to edit (and correct) the mutated gene, e.g., by introducing a replacement sequence (e.g., via delivery of a donor DNA). In some of the above cases (e.g., in cases related to treating Von Willebrand's disease, in cases related to targeting a cell harboring a mutation in the gene encoding VWF), a subject targeting ligand provides for targeted binding to E-selectin.

Methods and compositions of this disclosure can be used to treat any number of diseases, including any disease that is linked to a known causative mutation, e.g., a mutation in the genome. For example, methods and compositions of this disclosure can be used to treat sickle cell disease, B thalassemia, HIV, myelodysplastic syndromes, JAK2-mediated polycythemia vera, JAK2-mediated primary myelofibrosis, JAK2-mediated leukemia, and various hematological disorders. As additional non-limiting examples, the methods and compositions of this disclosure can also be used for B-cell antibody generation, immunotherapies (e.g., delivery of a checkpoint blocking reagent), and stem cell differentiation applications.

In some embodiments, a targeting ligand provides for targeted binding to KLS CD27+/IL-7Ra-/CD150+/CD34-hematopoietic stem and progenitor cells (HSPCs). For example, the beta-globin (HBB) gene may be targeted directly to correct the altered E7V substitution with an appropriate donor DNA molecule. As one illustrative example, a CRISPR/Cas RNA-guided polypeptide (e.g., Cas9, CasX, CasY, Cpf1) can be delivered with an appropriate guide RNA(s) such that it will bind to loci in the HBB gene and cut the genome, initiating insertion of an introduced donor DNA. In some cases, a Donor DNA molecule (single stranded or double stranded) is introduced (as part of a payload) and is release for 14-30 days while a guide RNA/CRISPR/Cas protein complex (a ribonucleoprotein complex) can be released over the course of from 1-7 days.

In some embodiments, a targeting ligand provides for targeted binding to CD4+ or CD8+ T-cells, hematopoietic stem and progenitor cells (HSPCs), or peripheral blood mononuclear cells (PBMCs), in order to modify the T-cell receptor. For example, a gene editing tool(s) (described elsewhere herein) can be introduced in order to modify the T-cell receptor. The T-cell receptor may be targeted directly and substituted with a corresponding homology-directed repair donor DNA molecule for a novel T-cell receptor. As one example, a CRISPR/Cas RNA-guided polypeptide (e.g., Cas9, CasX, CasY, Cpf1) can be delivered with an appropriate guide RNA(s) such that it will bind to loci in the HBB gene and cut the genome, initiating insertion of an introduced donor DNA. It would be evident to skilled artisans that other CRISPR guide RNA and donor sequences, targeting beta-globin, CCR5, the T-cell receptor, or any other gene of interest, and/or other expression vectors may be employed in accordance with the present disclosure.

In some cases, a subject method is used to target a locus that encodes a T cell receptor (TCR), which in some cases has nearly 100 domains and as many as 1,000,000 base pairs with the constant region separated from the V(D)J regions by 100,000 base pairs or more.

In some cases insertion of the donor DNA occurs within a nucleotide sequence that encodes a T cell receptor (TCR) protein. In some such cases the donor DNA encodes amino acids of a CDR1, CDR2, or CDR3 region of the TCR protein. See, e.g., Dash et al., Nature. 2017 Jul. 6; 547(7661):89-93. Epub 2017 Jun. 21; and Glanville et al., Nature. 2017 Jul. 6; 547(7661):94-98. Epub 2017 Jun. 21.

In some cases a subject method is used to insert genes while placing them under the control of (in operable linkage with) specific enhancers as a fail-safe to genome engineering. If the insertion fails, the enhancer is disrupted leading to the subsequent gene and any possible indels being unlikely to express. If the gene insertion succeeds, a new gene can be inserted with a stop codon at its end, which is particularly useful for multi-part genes such as the TCR locus. In some cases, the subject methods can be used to insert a chimeric antigen receptor (CAR) or other construct into a T-cell, or to cause a B-cell to create a specific antibody or alternative to an antibody (such as a nanobody, shark antibody, etc.).

In some cases the donor DNA includes a nucleotide sequence that encodes a chimeric antigen receptor (CAR). In some such cases, insertion of the donor DNA results in operable linkage of the nucleotide sequence encoding the CAR to an endogenous T-cell promoter (i.e., expression of the CAR will be under the control of an endogenous promoter). In some cases the donor DNA includes a nucleotide sequence that is operably linked to a promoter and encodes a chimeric antigen receptor (CAR)—and thus the inserted CAR will be under the control of the promoter that was present on the donor DNA.

In some cases the donor DNA includes a nucleotide sequence encoding a cell-specific targeting ligand that is membrane bound and presented extracellularly. In some cases, insertion of said donor DNA results in operable linkage of the nucleotide sequence encoding the cell-specific targeting ligand to an endogenous promoter. In some cases the donor DNA includes a promoter operably linked to the sequence that encodes a cell-specific targeting ligand that is membrane bound and presented extracellularly—and therefore, after insertion of the donor DNA, expression of the membrane bound targeting ligand will be under the control of the promoter that was present on the donor DNA.

In some embodiments, insertion of a donor DNA occurs within a nucleotide sequence that encodes a T cell receptor (TCR) Alpha or Delta subunit. In some cases, insertion of a donor DNA occurs within a nucleotide sequence that encodes a TCR Beta or Gamma subunit. In some cases a subject method and/or composition includes two donor DNAs. In some such cases insertion of one donor DNA occurs within a nucleotide sequence that encodes a T cell receptor (TCR) Alpha or Delta subunit and insertion of the other donor DNA occurs within a nucleotide sequence that encodes a T cell receptor (TCR) Beta or Gamma subunit.

In some embodiments, insertion of a donor DNA occurs within a nucleotide sequence that encodes a T cell receptor (TCR) Alpha or Delta subunit constant region. In some cases insertion of a donor DNA occurs within a nucleotide sequence that encodes a T cell receptor (TCR) Beta or Gamma subunit constant region. In some cases a subject method and/or composition includes two donor DNAs. In some such cases insertion of one donor DNA occurs within a nucleotide sequence that encodes a T cell receptor (TCR) Alpha or Delta subunit constant region and insertion of the other donor DNA occurs within a nucleotide sequence that encodes a T cell receptor (TCR) Beta or Gamma subunit constant region.

In some embodiments, insertion of a donor DNA occurs within a nucleotide sequence that functions as a T cell receptor (TCR) Alpha or Delta subunit promoter. In some cases insertion of a donor DNA occurs within a nucleotide sequence that functions as a T cell receptor (TCR) Beta or Gamma subunit promoter. In some cases a subject method and/or composition includes two donor DNAs. In some such cases insertion of one donor DNA occurs within a nucleotide sequence that functions as a T cell receptor (TCR) Alpha or Delta subunit promoter and insertion of the other donor DNA occurs within a nucleotide sequence that functions as a T cell receptor (TCR) Beta or Gamma subunit promoter.

In some embodiments, insertion of a sequence of the donor DNA occurs within a nucleotide sequence that encodes a T cell receptor (TCR) Alpha or Gamma subunit. In some cases, insertion of a sequence of the donor DNA occurs within a nucleotide sequence that encodes a TCR Beta or Delta subunit. In some cases a subject method and/or composition includes two donor DNAs. In some such cases insertion of one sequence of the donor DNA occurs within a nucleotide sequence that encodes a T cell receptor (TCR) Alpha or Gamma subunit and insertion of the sequence of the other donor DNA occurs within a nucleotide sequence that encodes a T cell receptor (TCR) Beta or Delta subunit.

In some embodiments, insertion of a sequence of the donor DNA occurs within a nucleotide sequence that encodes a T cell receptor (TCR) Alpha or Gamma subunit constant region. In some cases insertion of a sequence of the donor DNA occurs within a nucleotide sequence that encodes a T cell receptor (TCR) Beta or Delta subunit constant region. In some cases a subject method and/or composition includes two donor DNAs. In some such cases insertion of one sequence of the donor DNA occurs within a nucleotide sequence that encodes a T cell receptor (TCR) Alpha or Gamma subunit constant region and insertion of the sequence of the other donor DNA occurs within a nucleotide sequence that encodes a T cell receptor (TCR) Beta or Delta subunit constant region.

In some embodiments, insertion of a sequence of the donor DNA occurs within a nucleotide sequence that functions as a T cell receptor (TCR) Alpha or Gamma subunit promoter. In some cases insertion of a sequence of the donor DNA occurs within a nucleotide sequence that functions as a T cell receptor (TCR) Beta or Delta subunit promoter. In some cases a subject method and/or composition includes two donor DNAs. In some such cases insertion of one sequence of the donor DNA occurs within a nucleotide sequence that functions as a T cell receptor (TCR) Alpha or Gamma subunit promoter and insertion of the sequence of the other donor DNA occurs within a nucleotide sequence that functions as a T cell receptor (TCR) Beta or Delta subunit promoter.

In some embodiment, insertion of a donor DNA results in operable linkage of the inserted donor DNA with a T cell receptor (TCR) Alpha, Beta, Gamma or Delta endogenous promoter. In some cases, the donor DNA comprises a protein-coding nucleotide sequence that is operably linked to a TCR Alpha, Beta, Gamma or Delta promoter such that after insertion, the protein-coding sequence will remain operably linked to (under the control of) the promoter present in the donor DNA. In some cases insertion of said donor DNA results in operable linkage of the inserted donor DNA (e.g., a protein-coding nucleotide sequence such as a CAR, TCR-alpha, TCR-beta, TCR-gamma, or TCR-Delta sequence) with a CD3 or CD28 promoter. In some cases the donor DNA includes a protein-coding nucleotide sequence that is operably linked to a promoter (e.g., a T-cell specific promoter). In some cases insertion of the donor DNA results in operable linkage of the inserted donor DNA with an endogenous promoter (e.g., a stem cell specific or somatic cell specific endogenous promoter). In some cases the donor DNA includes a nucleotide sequence that encodes a reporter protein (e.g., fluorescent protein such as GFP, RFP, YFP, CFP, a near-IR and/or far red reporter protein, etc., e.g., for evaluating gene editing efficiency). In some cases the donor DNA includes a protein-coding nucleotide sequence (e.g., one that encodes all or a portion of a TCR protein) that does not have introns.

In some cases a subject method (and/or subject compositions) can be used for insertion of sequence for applications such as insertion of fluorescent reporters (e.g., a fluorescent protein such green fluorescent protein (GFP)/red fluorescent protein (RFP)/near-IR/far-red, and the like), e.g., into the C- and/or N-termini of any encoded protein of interest such as transmembrane proteins.

In some embodiments, insertion of the nucleotide sequence of the donor DNA into the cell's genome results in operable linkage of the inserted sequence with an endogenous promoter (e.g., (i) a T-cell specific promoter; (ii) a CD3 promoter; (iii) a CD28 promoter; (iv) a stem cell specific promoter; (v) a a somatic cell specific promoter; (vi) a T cell receptor (TCR) Alpha, Beta, Gamma or Delta promoter; (v) a B-cell specific promoter; (vi) a CD19 promoter; (vii) a CD20 promoter; (viii) a CD22 promoter; (ix) a B29 promoter; and (x) a T-cell or B-cell V(D)J-specific promoter). In some cases the nucleotide sequence, of the insert donor composition, that is inserted includes a protein-coding sequence that is operably linked to a promoter (e.g., (i) a T-cell specific promoter; (ii) a CD3 promoter; (iii) a CD28 promoter; (iv) a stem cell specific promoter; (v) a somatic cell specific promoter; (vi) a T cell receptor (TCR) Alpha, Beta, Gamma or Delta promoter; (v) a B-cell specific promoter; (vi) a CD19 promoter; (vii) a CD20 promoter; (viii) a CD22 promoter; (ix) a B29 promoter; and (x) a T-cell or B-cell V(D)J-specific promoter).

In some embodiments the nucleotide sequence that is inserted into the cell's genome encodes a protein. Any convenient protein can be encoded—examples include but are not limited to: a T cell receptor (TCR) protein; a CDR1, CDR2, or CDR3 region of a T cell receptor (TCR) protein; a chimeric antigen receptor (CAR); a cell-specific targeting ligand that is membrane bound and presented extracellularly; a reporter protein (e.g., a fluorescent protein such as GFP, RFP, CFP, YFP, and fluorescent proteins that fluoresce in far red, in near infrared, etc.). In some embodiments the nucleotide sequence that is inserted into the cell's genome encodes a multivalent (e.g., heteromultivalent) surface receptor (e.g., in some cases where a T-cell is the target cell). Any convenient multivalent receptor could be used and non-limiting examples include: bispecific or trispecific CARS and/or TCRs, or other affinity tags on immune cells. Such an insertion would cause the targeted cell to express the receptors. In some cases multivalence is achieved by inserting separate receptors whereby the inserted receptors function as an OR gate (one or the other triggers activation), or as an AND gate (receptor signaling is co-stimulatory and homovalent binding won't activate/stimulate cell, e.g., a targeted T-cell). A protein encoded by the inserted DNA (e.g., a CAR, a TCR, a multivalent surface receptor) can be selected such that it binds to (e.g., functions to target the cell, e.g., T-cell to) one or more targets selected from: CD3, CD8, CD4, CD28, CD90, CD45f, CD34, CD80, CD86, CD19, CD20, CD22, CD47, CD3-epsilon, CD3-gamma, CD3-delta; TCR Alpha, TCR Beta, TCR gamma, and/or TCR delta constant regions; 4-1BB, OX40, OX40L, CD62L, ARP5, CCR5, CCR7, CCR10, CXCR3, CXCR4, CD94/NKG2, NKG2A, NKG2B, NKG2C, NKG2E, NKG2H, NKG2D, NKG2F, NKp44, NKp46, NKp30, DNAM, XCR1, XCL1, XCL2, ILT, LIR, Ly49, IL2R, IL7R, IL 10R, IL12R, IL15R, IL18R, TNFα, IFNγ, TGF-β, and α5β1.

### Co-Delivery (not Necessarily a Nanoparticle of the Disclosure)

As noted elsewhere herein, one advantage of delivering multiple payloads as part of the same package (delivery vehicle) is that the efficiency of each payload is not diluted. In some embodiments a two different payloads are payloads of the same delivery vehicle. In some embodiments, a donor DNA and/or one or more gene editing tools (e.g., as described elsewhere herein) is delivered in combination with (e.g., as part of the same package/delivery vehicle, where the delivery vehicle does not need to be a nanoparticle of the disclosure) a protein (and/or a DNA or mRNA encoding same) and/or a non-coding RNA that increases genomic editing efficiency. In some embodiments, one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls cell division and/or differentiation. For example, in some cases one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls cell division. In some cases one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that controls differentiation. In some cases, one or more gene editing tools is delivered in combination with (e.g., as part of the same package/delivery vehicle, where the delivery vehicle does not need to be a nanoparticle of the disclosure) a protein (and/or a DNA or mRNA encoding same) and/or a non-coding RNA that biases the cell DNA repair machinery.

As noted above, in some cases the delivery vehicle does not need to be a nanoparticle of the disclosure. For example, in some cases the delivery vehicle is viral and in some cases the delivery vehicle is non-viral. Examples of non-viral delivery systems include materials that can be used to co-condense multiple nucleic acid payloads, or combinations of protein and nucleic acid payloads. Examples include, but are not limited to: (1) lipid based particles such as zwitterionic or cationic lipids, and exosome or exosome-derived vesicles; (2) inorganic/hybrid composite particles such as those that include ionic complexes co-condensed with nucleic acids and/or protein payloads, and complexes that can be condensed from cationic ionic states of Ca, Mg, Si, Fe and physiological anions such as O²⁻, OH, PO₄³⁻, SO₄²⁻; (3) carbohydrate delivery vehicles such as cyclodextrin and/or alginate; (4) polymeric and/or co-polymeric complexes such as poly(amino-acid) based electrostatic complexes, poly(Amido-Amine), and cationic poly(B-Amino Ester); and (5) virus like particles (e.g., protein and nucleic acid based). Examples of viral delivery systems include but are not limited to: AAV, adenoviral, retroviral, and lentiviral.

### Kits

Also within the scope of the disclosure are kits. For example, in some cases a subject kit can include one or more of (in any combination) any of the components discussed above, e.g.,: (i) a donor DNA; (ii) one or more gene editing tools; (iii) a targeting ligand, (iv) a linker, (v) a targeting ligand conjugated to a linker, (vi) a targeting ligand conjugated to an anchoring domain (e.g., with or without a linker), (vii) an agent for use as a sheddable layer (e.g., silica), (viii) a payload, e.g., a an siRNA or a transcription template for an siRNA or shRNA; a gene editing tool, a donor DNA, and the like, (ix) a polymer that can be used as a cationic polymer, (x) a polymer that can be used as an anionic polymer, (xi) a polypeptide that can be used as a cationic polypeptide, e.g., one or more HTPs, and (xii) a subject viral or non-viral delivery vehicle. In some cases, a subject kit can include instructions for use. Kits typically include a label indicating the intended use of the contents of the kit. The term label includes any writing, or recorded material, e.g., computer-readable media, supplied on or with the kit, or which otherwise accompanies the kit.

### Algorithmic Screening

Nanoparticle formulations have 13+parameters optimized for a specific payload and biological condition through iterative screening. These parameters include, but are not limited to (FIG. 13C):
Payload molar dose
Ratio of electric charge difference between payload compound and fully packaged particle
Ratio of electric charge difference between payload compound and anionic polymers (for a given full ratio)
Selection of library and/or variable cationic polymers
Molar ratio of cationic polymers (for a given selected cationic combination)
Selection of library and/or variable anionic polymers
Molar ratio of anionic polymers (for a given selected anionic combination)
D:L isomer ratio of one or more cationic components and/or cationic domains
D:L isomer ratio of one or more anionic components and/or anionic domains
Selection of diagnostically responsive ligand
Ligand surface density
Heteromultivalent combinations of up to four additional ligands (for a given surface density and primary ligand)
Selection of library ligand linker
Selection of library ligand anchor
Assembly order of compound addition
DNA/RNA/PNA/MNA/etc. and other identifiable sequences and/or multiplexed fluorophore barcoding (this includes gRNAs and donor DNAs with variable DNA/RNA/PNA/MNA/etc. barcodes on their ends)
Alternative means of studying a discrete range of nanomaterial properties as relates to self-assembly or colloidal suspension with a finite set of materials
Hydrophobic/water-oil-water/micellar techniques for NP synthesis with variable ligand coats (either directly conjugating to NP surface or through a peptide hydrophobic and/or hydrophilic domain that embeds in the hydrophobic and/or hydrophilic domain of a bilayer/monolayer of a liposome/micelle
   In some cases, size, charge, “condensation index”, and “release index” (ratio of transfected NP+ cells vs. functionally expressing/edited cells) are included as selection criteria for NP performance. For example, in some assays output is represented as the “condensation index”, which can be calculated as [(Well of Interest Fluorescence−Free DNA Fluorescence)/Free DNA Fluorescence] *100 and can be reported as average condensation index±standard deviation in a heatmap which correlates to the nanoparticle ID. More condensed nanoparticles will have higher shielding, less fluorescence, and thus a more negative condensation index

The number of all possible formulations even when limiting each parameter to only a few options becomes intractable for exhaustive screening. Several techniques can be employed to constrain the search heuristic, which integrates aspects of genetic algorithms, stochastic gradient descent, and simulated annealing. Screening consists of two phases: an initial ‘broad’ screen with generic formulations, followed by a set of ‘deep’ iterative screens.

The first phase of screening samples a diverse set of possible particle architectures to sparsely cover the entire search space with initial values. The initial formulations are a combination of preformulated benchmark particles and generated formulations with uniform step changes in a given parameter. Characterization of these initial formulations in terms of physicochemical properties (such as diameter and charge) and biological activity (such as uptake percentage, uptake rate, gene expression, and toxicity) provides a data signature of the particles, the components of which are individually weighted and summed with a performance scoring function.

For optimization purposes, a particle can be described as being a feature vector in formulation parameter space that an unknown function maps to a vector in scoring space. The objective of an iterative optimization strategy would then be to increment a formulation's parameters to increase and ultimately maximize a particle's score. Subsequent rounds of optimization utilize this paradigm. A machine learning-based approach can be used to both approximate the unknown objective function and generate changes to candidate formulations. In this phase of screening, candidate formulations can be robotically synthesized, characterized, and a subset of top performers can be selected. In the simplest embodiment, this subset can be a threshold percentage of the highest aggregate scores. In other cases, selection and deselection criteria can be used to filter the list of candidate formulations. Example criteria are selecting no particles with diameter above 600 um, or selecting particles with a lower aggregate score if their expression efficiency is in the top 10% of the round. Each formulation in this subset can then be iterated into several variations incrementing different parameters to generate the next full round of candidate formulations.

The algorithm uses the error difference between predicted performance and measured performance, in addition to the accumulation of data points from all previous rounds of screening, to refine the estimation of the objective function leading to improved predictions and optimizations over time. As rounds progress, the size of the parameter change from a parent formulation to its offspring formulations is progressively limited to allow for stable convergence and finer optimization. This method facilitates reasonably optimal formulations in an exponential search space while being sufficiently efficient to achieve rapid turnaround.

tSNE (t-Distributed Stochastic Neighbor Embedding), PCA (Principal component analysis) and other forms of modeling nanoparticle multiparametric data via unsupervised learning (e.g., input=formulation, output=bio and nano characterization) can be used, whereby top performing and/or “most interesting” formulation clusters (i.e., formulation clusters of interest) are automatically selected and iterated around (e.g., for one or more additional rounds of screening). In some such cases, a nanoparticle or gene barcode can be used as as one of the variables in the method (e.g., tSNE), where one can optionally investigate data such as mRNA-Seq data, and then aggregate how each specific cell sub population type behaves with the nanoparticle in terms of any desired parameter(s) (e.g., survival, uptake, expression, and the like).

Theranostics

Theranostic (e.g. MRI, PET or CT contrast agent) nanoparticles may be utilized to determine biodistributions of given targeting ligand approaches. The nanoparticles may also be fluorescently labeled with near IR, far red or other dyes in order to be used for in vivo fluorescent imaging, or determination of uptake following biopsy of blood/cells/tissue(s)/organ(s). Gadolinium and other MRI/PET/CT contrast agents may also be tethered to ligands to establish baseline human biodistributions of ligand-targeting approaches. A library of “diagnostically-responsive” nanoparticles may be administered to the patient following a diagnosis, and a secondary biopsy or in vivo imaging technique (as detailed above) may be used to determine which variants achieved the desired uptake/expression in a given cell population or distribution to a given tissue/organ population. Subsequently, therapeutic modalities may be administered utilizing theranostically-identified ligand variants.

Other Uses

Generating Drug-Peptide Conjugates
Covalent small molecule or biologic drug tethering to side chains of carrier polymers
Inclusion of various drugs or biologics as direct covalent conjugates to targeting ligands
Enhanced cell-type-specific screening for any alternative targeting approach (e.g. SELEX, phage display, antibody conjugation to nanoparticles), especially where heterovalent (2+targeting ligands) embodiments lead to greater specificity or where predictive data minimizes off-target effects while maximizing specificity, even if a homovalent approach (1 targeting ligand) is used
Use of targeting ligands for diagnostic purposes, such as upon the surfaces of chips (e.g. SPR, microfluidic rolling assays, or an electrically-modulated avid grid), in order to create cell-selection and cell-targeting approaches by chip-based assays

### Techniques for Assessing Physicochemical and Biological Performance of Top Nanoparticle Formulations

In all experiments, the following instrumentation was used:
Genomics: Sanger sequencing was outsourced to GENEWIZ following PCR amplification of target genetic loci, and uploaded to Synthego's ICE analysis tool in parallel to internal computational data evaluation
Flow Cytometer: Attune NxT with Flow Cytometer

### Microscopy: BioTek Cytation V

### Particle Sizes and Zeta Potentials: Wyatt Mobius

### Transmission Electron Microscopy: LVEM5 (Delong America)

### Particle Synthesis: Andrew (Andrew Alliance)

### Transfections and Cell Media Handling: OpenTrons OT-2

### Fluorimetry and SYBR Assays: BioTek H1 Reader

### First Illustrative Example of Nanoparticle Synthesis

Procedures were performed within a sterile, dust free environment (BSL-II hood). Gastight syringes were sterilized with 70% ethanol before rinsing 3 times with filtered nuclease free water, and were stored at 4° C. before use. Surfaces were treated with RNAse inhibitor prior to use.

### Nanoparticle Core

A first solution (an anionic solution) was prepared by combining the appropriate amount of payload (in this case plasmid DNA (EGFP-N1 plasmid) with an aqueous mixture (an ‘anionic polymer composition’) of poly(D-glutamic Acid) and poly(L-glutamic acid). This solution was diluted to the proper volume with 10 mM Tris-HCl at pH 8.5. A second solution (a cationic solution), which was a combination of a ‘cationic polymer composition’ and a ‘cationic polypeptide composition’, was prepared by diluting a concentrated solution containing the appropriate amount of condensing agents to the proper volume with 60 mM HEPES at pH 5.5. In this case, the ‘cationic polymer composition’ was poly(L-arginine) and the ‘cationic polypeptide composition’ was 16 μg of H3K4(me3) (tail of histone H3, tri methylated on K4).

Precipitation of nanoparticle cores in batches less than 200 μl can be carried out by dropwise addition of the condensing solution to the payload solution in glass vials or low protein binding centrifuge tubes followed by incubation for 30 minutes at 4° C. For batches greater than 200 μl, the two solutions can be combined in a microfluidic format (e.g., using a standard mixing chip (e.g. Dolomite Micromixer) or a hydrodynamic flow focusing chip). Optimal input flowrates can be determined such that the resulting suspension of nanoparticle cores is monodispersed, exhibiting a mean particle size below 100 nm. In many embodiments, a robotic fluid handling approach is utilized to perform sequential addition of peptides to payloads as detailed elsewhere.

In one case, the two equal volume solutions from above (one of cationic condensing agents and one of anionic condensing agents) were prepared for mixing. For the solution of cationic condensing agents, polymer/peptide solutions were added to one protein low bind tube (eppendorf) and were then diluted with 60 mM HEPES (pH 5.5) to a total volume of 100 μl (as noted above). This solution was kept at room temperature while preparing the anionic solution. For the solution of anionic condensing agents, the anionic solutions were chilled on ice with minimal light exposure. 10 μg of nucleic acid in aqueous solution (roughly 1 μg/μl) and 7 μg of aqueous poly (D-Glutamic Acid) [0.1%] were diluted with 10 mM Tris-HCl (pH 8.5) to a total volume of 100 μl (as noted above).

Each of the two solutions was filtered using a 0.2 micron syringe filter and transferred to its own Hamilton 1 ml Gastight Syringe (Glass, (insert product number). Each syringe was placed on a Harvard Pump 11 Elite Dual Syringe Pump. The syringes were connected to appropriate inlets of a Dolomite Micro Mixer chip using tubing, and the syringe pump was run at 120 μl/min for a 100 μl total volume. The resulting solution included the core composition (which now included nucleic acid payload, anionic components, and cationic components).

### Core Stabilization (Adding a Sheddable Layer)

To coat the core with a sheddable layer, the resulting suspension of nanoparticle cores was then combined with a dilute solution of sodium silicate in 10 mM Tris HCl (pH8.5, 10-500 mM) or calcium chloride in 10 mM PBS (pH 8.5, 10-500 mM), and allowed to incubate for 1-2 hours at room temperature. In this case, the core composition was added to a diluted sodium silicate solution to coat the core with an acid labile coating of polymeric silica (an example of a sheddable layer). To do so, 10 μl of stock Sodium Silicate (Sigma) was first dissolved in 1.99 ml of Tris buffer (10 mM Tris pH=8.5, 1:200 dilution) and was mixed thoroughly. The Silicate solution was filtered using a sterile 0.1 micron syringe filter, and was transferred to a sterile Hamilton Gastight syringe, which was mounted on a syringe pump. The core composition from above was also transferred to a sterile Hamilton Gastight syringe, which was also mounted on the syringe pump. The syringes were connected to the appropriate inlets of a Dolomite Micro Mixer chip using PTFE tubing, and the syringe pump was run at 120 μl/min. In other embodiments, poly(glutamic acid) (0.1% and 0.15% w/v) in either pH 5.5 HEPES or pH 7.4 Tris was utilized following the initial core formation in place of silica.

Stabilized (coated) cores can be purified using standard centrifugal filtration devices (100 kDa Amicon Ultra, Millipore) or dialysis in 30 mM HEPES (pH 7.4) using a high molecular weight cutoff membrane. In many cases, no purification is necessary following electrostatic assembly. In the case of silica-coated particles, the stabilized (coated) cores were purified using a centrifugal filtration device. The collected coated nanoparticles (nanoparticle solution) were washed with dilute PBS (1:800) or HEPES and filtered again (the solution can be resuspended in 500 μl sterile dispersion buffer or nuclease free water for storage). Effective silica coating was demonstrated. The stabilized cores had a size of 110.6 nm and zeta potential of −42.1 mV (95%).

### Surface Coat (Outer Shell)

Addition of a surface coat (also referred to as an outer shell), sometimes referred to as “surface functionalization,” was accomplished by electrostatically grafting ligand species (in this case Rabies Virus Glycoprotein fused to a 9-Arg peptide sequence as a cationic anchoring domain—‘RVG9R’) to the negatively charged surface of the stabilized (in this case silica coated) nanoparticles. Beginning with silica coated nanoparticles that were filtered and resuspended in dispersion buffer or water, the final volume of each nanoparticle dispersion was determined, as was the desired amount of polymer or peptide to add such that the final concentration of protonated amine group was at least 75 uM. The desired surface constituents were added and the solution was sonicated for 20-30 seconds prior to incubate for 1 hour. Centrifugal filtration was performed at 300 kDa (the final product can be purified using standard centrifugal filtration devices, e.g., 300-500 kDa from Amicon Ultra Millipore, or dialysis, e.g., in 30 mM HEPES (pH 7.4) using a high molecular weight cutoff membrane), and the final resuspension was in either cell culture media or dispersion buffer. In some cases, optimal outer shell addition yields a monodispersed suspension of particles with a mean particle size between 50 and 150 nm and a zeta potential between 0 and −10 mV. In this case, the nanoparticles with an outer shell had a size of 115.8 nm and a Zeta potential of −3.1 mV (100%).

### Second Illustrative Example of Nanoparticle Synthesis

Nanoparticles were synthesized at room temperature, 37C or a differential of 37C and room temperature between cationic and anionic components. Solutions were prepared in aqueous buffers utilizing natural electrostatic interactions during mixing of cationic and anionic components. At the start, anionic components were dissolved in Tris buffer (30 mM-60 mM; pH=7.4-9) or HEPES buffer (30 mM, pH=5.5) while cationic components were dissolved in HEPES buffer (30 mM-60 mM, pH=5-6.5).

Specifically, payloads (e.g., genetic material (RNA or DNA), genetic material-protein-nuclear localization signal polypeptide complex (ribonucleoprotein), or polypeptide) were reconstituted in a basic, neutral or acidic buffer. For analytical purposes, the in some experiments the payload was manufactured to be covalently tagged with or genetically encode a fluorophore. With pDNA payloads, a Cy5-tagged peptide nucleic acid (PNA) specific to AGAGAG tandem repeats was used to fluorescently tag fluorescent reporter vectors and fluorescent reporter-therapeutic gene vectors. A timed-release component that may also serve as a negatively charged condensing species (e.g. poly(glutamic acid)) was also reconstituted in a basic, neutral or acidic buffer. Targeting ligands with a wild-type derived or wild-type mutated targeting peptide conjugated to a linker-anchor sequence were reconstituted in acidic buffer. In the case where additional condensing species or nuclear localization signal peptides were included in the nanoparticle, these were also reconstituted in buffer as 0.03% w/v working solutions for cationic species, and 0.015% w/v for anionic species. Experiments were also conducted with 0.1% w/v working solutions for cationic species and 0.1% w/v for anionic species. All polypeptides, except those complexing with genetic material, were sonicated for ten minutes to improve solubilization.

### Illustrative Example of Iterative Nanoparticle Synthesis:

Rationale: In the previous experiments (FIGS. 19F-19L), high nanoparticle uptake was observed in Unstimulated T-Cells by flow cytometry that did not translate to good ICE or knockout (KO) scores with downstream Sanger sequencing (all 0% and 1%). This is likely related to RNPs being taken up by cells but unable to release the RNP payload inside the cell, resulting in poor ICE scores. The amount of endosomal escape peptide added to the NPs was then titrated to identify the right concentration to facilitate intracellular release of payload, and optimize H2A-3C vs. H2B-3C vs. PLR10 concentrations for initial RNP stabilization into a uniformly cationic surface for subsequent multilayered assembly of nanoparticles.

### General Methods:

### Stimulated T-Cells and HEK293

RNP=Cas9+LL224 (TRAC) guide
2 NP Prep Plates: single-layer and multi-layer
Overnight (˜12 h) transfection
Transfection in serum free media
blow Day 1 (uptake)—all

### T-Cell Flow Day 4 & Day 7 (TCR KD)

### T-Cell Genomics Day 4 & Day 7

HEK293 Genomics Day 3/4 (TRAC editing)—grew out to Day 7 for genomics
Order of addition:
Order 1—RNP>[H2A>PLE/PDE layer]>EED>LIGAND
Order 2—RNP>[H2A>PLE/PDE layer]>LIGAND>EED
Order 3—RNP>[H2A>PLE/PDE layer]>LIGAND/EED
Dose of EE peptide: (0, 0.15, 0.3) molar ratio

### Multilayer “Andrew” Particles:

3 orders of addition
3 EE Concentration (0, 0.15 0.3 mole fraction), all using AF594 tagged EE peptide+Stock EE AF594 is at 0.1%

### 1 RNP: Cas9-GFP+sgLL224

All at charge ratio 10 (Corresponding to Column 6 and 8 from 3B.2.1.1 prep plate, CD8-PLR9, 1 transfection time (overnight), with 10 particles=5 cpp (cationic polypeptide)×2 app (anionic polypeptide). See FIGS. 19E and 19G-19F for precise robotic instructions of each nanoparticle formulation.

### Single Layer “Handmix” Particles:

2 nucleases
3 orders of addition
3 EE Doses (0, 0.15, 0.3 mole fraction)
5 ligands—CD8-Peg-9R, CD8-9R, PLR10, PLK10-PEG22, CD4-9R
1 transfection time (overnight)
One Buffer (HEPES pH 5.5)—this buffer produced slightly better ICE scores in the 3B.1.1.1 HEK-GFP cells See FIGS. 19T and 19U for detailed nanoparticle formulations.
Enhancing the Cutting Efficiency of Cas9 Protein through Systematic Nanoparticle Formulation: Data Driven Example

For many of the embodiments shown herein, the effect that different buffers and pH levels have on Cas9 aggregation was evaluated prior to formation of subsequent nanoparticles (FIG. 19A). The purpose of this study was to develop an ideal nanoparticle formulation that effectively delivers functional Cas9 protein to T cells using our iterative platform. This process included several rounds of analysis and treatment of the payload, determination of the nanoparticle layers and their mixing order, and establishment of varying charge and molar ratios of each layer. Nanoparticles were characterized through size, zeta potential, and stability, and cutting efficacy was determined through inference of CRISPR Edits (ICE) analysis.
The initial rounds of experiments were intended to assess the protein of interest, Cas9. The first few experiments considered the treatment of Cas9 by filtration and centrifugation. Cas9 was either filtered through 0.1 micron, 0.2 micron, 100 kDa, and 300 kDa filters or centrifuged, or not filtered at all. The size dispersity of the protein was then measured to determine which treatment of lead to the highest population of monomer, dimer, and trimer Cas9 (least aggregated).
The effect of agitation, sonication, shearing, and vortexing on Cas9 aggregation was also analyzed in addition to the buffer conditions evaluated in FIG. 19A. We evaluated various factors on the aggregation and efficacy of Cas9 ribonucleoprotein (RNP) prior to NP formation. Different permutations of RNP formulations were tested, and a final method was locked for the following nanoparticle synthesis studies.
Using computer-assisted formulation design, we evaluated the physicochemical properties of single-layered DNA (payload+outer layer) and multi-layered (payload+layer 1+layer 2++layer n) nanoparticles as a baseline for Cas9 nanoparticle synthesis (FIG. 19B). Condensation of the payload of the nanoparticles was evaluated using a SYBR Gold assay. Delta in fluorescence is calculated as—{(Fluorescence value for sample at time x-fluorescence value of naked plasmid or dsDNA controls at time x)/fluorescence value of naked plasmid or dsDNA controls at time x)}*100 and can be seen for each formulation (FIG. 19C). Sizes and zeta potentials of associated particles are shown in FIGS. 19D and 19E, respectively.
Using this experiment, another round of computer assisted formulation was conducted to generate single layered RNP nanoparticles (FIGS. 19F1-2). The physicochemical properties (FIGS. 19G-19H) and downstream cutting efficacy (FIG. 19I) of these nanoparticles were evaluated. Cutting efficacy via ICE was low for the single-layered NPs at this stage, aside from the positive control.
A similar experiment (FIG. 19J) was conducted using computer assisted formulation to generate and characterize multi-layered Cas9 nanoparticles. In this experiment, the order of addition of each layer was also investigated. These orders included:
   A. CPP>RNP>DNA+PLE mix>PLR10
   B. DNA+PLE mix>CPP>RNP>PLR10

### C. DNA>CPP>PLE>PLR10

D. RNP+DNA>CPP>PLE>PLR10 (control group)

### E. RNP>CPP>PLE>PLR10

F. DNA+PLE mix>CPP+RNP mix>PLR10
G. CPP+RNP mix>DNA+PLE mix>PLR10
Nanoparticle behavior in serum was also evaluated to determine groups with optimal nanoparticle designs (FIG. 19K). Cutting efficacy via ICE was low for the multi layered NPs at this stage (FIG. 19L).
Using data from the previous experiments, computer assisted formulation was used in another round to enhance nanoparticle efficacy. These nanoparticles were then used to transfect both stimulated and unstimulated T cells in serum or serum free media (FIG. 19M). Physicochemical properties (predicted charge ratios), payloads, ligands and transfected cell types of each component are displayed in FIG. 19N.
   The nanoparticles shown in FIG. 19N were able to be delivered and perform cuts effectively to T-cells. Physicochemical properties of nanoparticles are shown in FIGS. 19O and 19P. Summary of all ICE scores (C11, D11, E11, and F11 are nucleofection positive controls) are shown in FIGS. 19Q-19R.
   Once nanoparticle cores have been iterated and consolidated for a certain payload, a similar iteration process follows for the nanoparticle ligand surface based on the specific cell of interest. In the enclosed examples, a variety of surface ligands were iterated through to target either T cells generally, or subpopulation of T cells such as CD4+ or CD8+ specifically.
   Multiparametric datasets that can be used as selection criteria for machine learning and human-assisted design of experiments can be seen in FIG. 19S.
   In the plate of formulations depicted in FIG. 19V, a constant nanoparticle core was used and T-cell specific ligands were iterated over with various orders of addition. The heatmaps depict the percent uptake of each unique formulation in a live cell population (CD4+vs CD8+pan-T cells) as determined by flow cytometry, and the associated particle sizes and zeta potentials (FIGS. 19W-19Y). Breakdown of the data shows that the T cell specific ligand composition was more effective in being taken up by the cells compared to a general cell penetrating peptide. Additionally, the surface ligands had a preference for CD4+ cells vs CD8+ were able to achieve ˜10-fold selectivity for CD4+ T-cells vs. CD8+ T cells.
Sanger sequencing and ICE (inference of CRISPR edits) analysis of top nanoparticle groups in human primary Pan T cells can be seen in FIGS. 19R and 19Z.
Optimization of CRISPR Cas9 RNP sizes can be seen with a zwitterionic charge homogenizing techniques as shown in FIG. 19ZA.

### Exemplary Heteromultivalent Robotic Screen

In the following flow cytometry data, an Attune NxT flow cytometer was used to determine cellular uptake of EGFP-Cas9 RNPs formed with a variety of heteromultivalent ligand coats transfected in human primary T cells with flow cytometry performed at 24 h. These studies were performed prior to subsequent core and ligand density optimization studies where cellular transfection efficiencies of Cas9 RNP-bearing nanoparticles exceeds 90% in CD4+ T cells. In these initial experiments, in human primary T cells as well as AF594 AND GFP+ cells following formulator app generated robotic code (FIGS. 13E-13J). Subsequent optimization (FIGS. 19A-19F) led to substantial increases in cellular transfection efficiency and gene editing efficiency. Recursive automation, rapid peptide synthesis and integrated robotic platforms allows for screening a tremendous state-space of possible formulations to identify an optimal “hit.”

| | | % | % | % | % | Median | % | % | % | % | |
|---|---|---|---|---|---|---|---|---|---|---|---|
| Cell | Cells_Count | Live | CD4+_LIVE | CD8+_LIVE | GFP_LIVE | SIG FP | GFP_CD8 | GFP_CD4 | GFP(CD8-CD4) | Alexa594_GFP+ | Ligand_1 |
| C10 | 4978 | 67.4 | 69.5 | 26.5 | 8.39 | 5146 | 8.94 | 7.7 | 1.24 | 1.1 | cl23_CD8_xxx_4GS_2_9R_N_1 |
| C11 | 5523 | 66.5 | 70.5 | 25.4 | 5.3 | 7697 | 4.86 | 5.02 | −0.16 | 11.1 | cl1_CD45_mSiglec_4GS_2_9R_C_1 |
| C12 | 7646 | 71.5 | 70 | 25.7 | 0.93 | 4172 | 1.1 | 0.76 | 0.34 | 24.5 | cl1_CD45_mSiglec_4GS_2_9R_C_1 |
| C13 | 4558 | 68.7 | 70.2 | 25.1 | 5.77 | 5972 | 6.31 | 5.45 | 0.86 | 6.29 | cl1_CD45_mSiglec_4GS_2_9R_C_1 |
| C14 | 5963 | 56.4 | 71.7 | 23.2 | 16.1 | 3952 | 15.5 | 16 | −0.5 | 27.4 | cl1_CD45_mSiglec_4GS_2_9R_C_1 |
| C15 | 4683 | 73.6 | 70.7 | 25.2 | 1.63 | 4696 | 1.43 | 1.66 | −0.23 | 31.5 | cl1_CD45_mSiglec_4GS_2_9R_C_1 |
| C16 | 4714 | 67.5 | 70.3 | 25.8 | 6.87 | 5891 | 7.06 | 6.21 | 0.85 | 6.64 | cl7_CD137_m41BBlg_4GS_2_9R_N_1 |
| C17 | 5965 | 71.4 | 71 | 25 | 2.74 | 2022 | 4.01 | 2.38 | 1.63 | 18.8 | cl7_CD137_m41BBlg_4GS_2_9R_N_1 |
| C18 | 5299 | 60 | 70 | 25.6 | 6.25 | 6954 | 7.64 | 5.35 | 2.29 | 54.2 | cl7_CD137_m41BBlg_4GS_2_9R_N_1 |
| C19 | 3791 | 66.9 | 68.1 | 28 | 9.77 | 5872 | 12.9 | 9.52 | 3.38 | 52.9 | cl12_IL2R_mIL2_4GS_2_9R_N_1 |
| C3 | 7055 | 61.8 | 72.2 | 24.2 | 16.6 | 5042 | 17.4 | 16 | 1.4 | 38.8 | cl23--CD8_rmNEF_4GS_2_9R_N_1 |
| C4 | 7446 | 62.5 | 71.4 | 24.4 | 5.75 | 5734 | 5.05 | 5.9 | −0.85 | 16.5 | cl23_CD8_xxx_4GS_2_9R_N_1 |
| C5 | 6650 | 63.3 | 71.2 | 23.6 | 16.4 | 6588 | 14.5 | 16.2 | −1.7 | 4.13 | cl23_CD8_xxx_4GS_2_9R_N_1 |
| C6 | 8631 | 69.6 | 70.5 | 25.2 | 7.31 | 4060 | 8.11 | 6.69 | 1.42 | 17.4 | cl23_CD8_xxx_4GS_2_9R_N_1 |
| C7 | 6384 | 61.6 | 68.4 | 26.1 | 21.4 | 6094 | 19 | 21.3 | −2.3 | 15.2 | cl23_CD8_xxx_4GS_2_9R_N_1 |
| C8 | 6689 | 61.1 | 69.9 | 23.8 | 34.8 | 6954 | 33.2 | 33.8 | −0.6 | 5.81 | cl23_CD8_xxx_4GS_2_9R_N_1 |
| C9 | 4868 | 67.1 | 70.4 | 24.8 | 19.1 | 6861 | 20.1 | 17.4 | 2.7 | 11.6 | cl23_CD8_xxx_4GS_2_9R_N_1 |
| D10 | 7329 | 65.5 | 69.3 | 26 | 14.4 | 4680 | 14.3 | 14.2 | 0.1 | 1.79 | cl23_CD8_xxx_4GS_2_9R_N_1 |
| D11 | 5798 | 70.6 | 69.2 | 26.3 | 4.93 | 3992 | 6.58 | 4.3 | 2.28 | 9.79 | cl1_CD45_mSiglec_4GS_2_9R_C_1 |
| D12 | 7491 | 73 | 70.3 | 25.7 | 1.25 | 2914 | 1.85 | 1 | 0.85 | 28.8 | cl1_CD45_mSiglec_4GS_2_9R_C_1 |
| D13 | 4754 | 68.3 | 70.7 | 25.2 | 3.2 | 2460 | 4.31 | 2.99 | 1.32 | 4 | cl1_CD45_mSiglec_4GS_2_9R_C_1 |
| D14 | 6308 | 64.9 | 69 | 26.7 | 3.87 | 4841 | 4.21 | 3.58 | 0.63 | 7.74 | cl1_CD45_mSiglec_4GS_2_9R_C_1 |
| D15 | 3198 | 67.9 | 70.2 | 25.3 | 5.48 | 1103 | 8.4 | 4.91 | 3.49 | 53.4 | cl1_CD45_mSiglec_4GS_2_9R_C_1 |
| D16 | 4495 | 61.8 | 69.1 | 26.6 | 7.79 | 8434 | 6.92 | 7.61 | −0.69 | 41 | cl7_CD137_m41BBlg_4GS_2_9R_N_1 |
| D17 | 5713 | 62.4 | 69.4 | 26.3 | 4.35 | 608 | 6.11 | 4.25 | 1.86 | 4.79 | cl7_CD137_m41BBlg_4GS_2_9R_N_1 |
| D18 | 5949 | 68 | 68.4 | 27.2 | 5.84 | 5111 | 5.95 | 5.87 | 0.08 | 6.17 | cl7_CD137_m41BBlg_4GS_2_9R_N_1 |
| D19 | 5113 | 62.2 | 70.9 | 25 | 5.18 | 5323 | 5.6 | 5.05 | 0.55 | 8.18 | cl12_IL2R_mIL2_4GS_2_9R_N_1 |
| D3 | 6675 | 64.1 | 71.3 | 25.9 | 2.74 | 6931 | 2.58 | 2.84 | −0.26 | 63.5 | cl1--CD45_mSiglec_4GS_2_9R_C_1 |
| D4 | 8023 | 62.7 | 69.7 | 26.1 | 6.31 | 5891 | 6.8 | 5.99 | 0.81 | 6.56 | cl23_CD8_xxx_4GS_2_9R_N_1 |
| D5 | 7249 | 62.2 | 70.2 | 25.4 | 19.8 | 7366 | 20.2 | 19.2 | 1 | 6.56 | cl23_CD8_xxx_4GS_2_9R_N_1 |
| D6 | 7452 | 60.1 | 70.7 | 24.6 | 11.3 | 913 | 14.8 | 10.6 | 4.2 | 10 | cl23_CD8_xxx_4GS_2_9R_N_1 |
| D7 | 4336 | 64.8 | 71.5 | 24 | 8.98 | 8784 | 11.1 | 7.64 | 3.46 | 31 | cl23_CD8_xxx_4GS_2_9R_N_1 |
| D8 | 6478 | 60.6 | 68.9 | 26 | 15.4 | 7267 | 16.9 | 14.2 | 2.7 | 9.95 | cl23_CD8_xxx_4GS_2_9R_N_1 |
| D9 | 5052 | 63.7 | 68.2 | 25.6 | 31.1 | 604 | 30.9 | 31 | −0.1 | 8.02 | cl23_CD8_xxx_4GS_2_9R_N_1 |
| E10 | 5190 | 63.3 | 67.7 | 27.9 | 8.98 | 7543 | 8.72 | 8.29 | 0.43 | 21.2 | cl23_CD8_xxx_4GS_2_9R_N_1 |
| E11 | 5306 | 59 | 70.9 | 23.4 | 15.5 | 6884 | 16.4 | 14.7 | 1.7 | 47 | cl1_CD45_mSiglec_4GS_2_9R_C_1 |
| E12 | 4487 | 61.5 | 70 | 26.1 | 4.8 | 8292 | 6.04 | 3.97 | 2.07 | 57 | cl1_CD45_mSiglec_4GS_2_9R_C_1 |
| E13 | 3299 | 59.8 | 69.2 | 24 | 21.5 | 6012 | 20.8 | 19.8 | 1 | 43.3 | cl1_CD45_mSiglec_4GS_2_9R_C_1 |
| E14 | 4109 | 62.3 | 67.7 | 27.9 | 6.09 | 1052 | 7.36 | 5.78 | 1.58 | 19.9 | cl1_CD45_mSiglec_4GS_2_9R_C_1 |
| E15 | 4596 | 59.7 | 68.1 | 26.2 | 16.8 | 5911 | 16.8 | 15.8 | 1 | 58.6 | cl1_CD45_mSiglec_4GS_2_9R_C_1 |
| E16 | 4855 | 63.2 | 69.1 | 26.7 | 5.59 | 8994 | 5.61 | 4.91 | 0.7 | 11.9 | cl7_CD137_m41BBlg_4GS_2_9R_N_1 |
| E17 | 5568 | 57.9 | 69.5 | 25.2 | 18.1 | 5216 | 15.3 | 18.2 | −2.9 | 23.5 | cl7_CD137_m41BBlg_4GS_2_9R_N_1 |
| E18 | 4573 | 66.5 | 70.2 | 25.2 | 3.54 | 7671 | 4.58 | 3.39 | 1.19 | 43.3 | cl7_CD137_m41BBlg_4GS_2_9R_N_1 |
| E19 | 4101 | 60.9 | 67.5 | 27.8 | 12.5 | 9272 | 10.8 | 12.5 | −1.7 | 38.4 | cl12_IL2R_mIL2_4GS_2_9R_N_1 |
| E3 | 5756 | 68.2 | 70.5 | 25 | 3.89 | 7517 | 5.19 | 3 | 2.19 | 74.1 | cl7--CD137_m41BBlg_4GS_2_9R_N_1 |
| E4 | 6114 | 61.2 | 69.2 | 26.2 | 8.32 | 8724 | 7.21 | 7.98 | −0.77 | 26.6 | cl23_CD8_xxx_4GS_2_9R_N_1 |
| E5 | 5732 | 60.3 | 68.3 | 25.5 | 21.3 | 9367 | 17.9 | 20.7 | −2.8 | 13.3 | cl23_CD8_xxx_4GS_2_9R_N_1 |
| E6 | 5067 | 64 | 69.6 | 25.2 | 20.7 | 10298 | 19.9 | 19.4 | 0.5 | 10.5 | cl23_CD8_xxx_4GS_2_9R_N_1 |
| E7 | 7200 | 64.1 | 71.5 | 23 | 15.5 | 9624 | 13.5 | 14.5 | −1 | 30.8 | cl23_CD8_xxx_4GS_2_9R_N_1 |
| E8 | 4626 | 58.9 | 69.8 | 25.1 | 18.4 | 9086 | 15.5 | 18.3 | −2.8 | 14.4 | cl23_CD8_xxx_4GS_2_9R_N_1 |
| E9 | 4415 | 61.2 | 68.3 | 25.2 | 26.5 | 4540 | 22.9 | 26.8 | −3.9 | 5.61 | cl23_CD8_xxx_4GS_2_9R_N_1 |
| F10 | 5474 | 61.1 | 68.5 | 26.5 | 22 | 4680 | 22.2 | 20.9 | 1.3 | 19.6 | cl23_CD8_xxx_4GS_2_9R_N_1 |
| F11 | 5603 | 64 | 69.3 | 26 | 7 | 6521 | 8.73 | 5.95 | 2.78 | 7.05 | cl1_CD45_mSiglec_4GS_2_9R_C_1 |
| F12 | 4027 | 65 | 69 | 26.7 | 2.71 | 9821 | 3.38 | 2.16 | 1.22 | 39.1 | cl1_CD45_mSiglec_4GS_2_9R_C_1 |
| F13 | 4576 | 65.9 | 68.7 | 26.5 | 8.14 | 5008 | 8.44 | 7.8 | 0.64 | 2.95 | cl1_CD45_mSiglec_4GS_2_9R_C_1 |
| F14 | 3630 | 60.7 | 70.3 | 25.1 | 14.7 | 9055 | 16 | 12.9 | 3.1 | 33.3 | cl1_CD45_mSiglec_4GS_2_9R_C_1 |
| F15 | 5468 | 70.6 | 69.9 | 25.5 | 1.86 | 2865 | 2.74 | 1.46 | 1.28 | 24.6 | cl1_CD45_mSiglec_4GS_2_9R_C_1 |
| F16 | 4081 | 65 | 68.5 | 26.8 | 9.42 | 6198 | 10.1 | 8.47 | 1.63 | 11.6 | cl7_CD137_m41BBlg_4GS_2_9R_N_1 |
| F17 | 5307 | 62.2 | 69.1 | 25.3 | 10 | 1118 | 9.51 | 10.1 | −0.59 | 2.22 | cl7_CD137_m41BBlg_4GS_2_9R_N_1 |
| F18 | 4236 | 60.3 | 66.9 | 26.8 | 10.6 | 5972 | 8.84 | 10.8 | −1.96 | 53.1 | cl7_CD137_m41BBlg_4GS_2_9R_N_1 |
| F19 | 4922 | 65.4 | 71.7 | 24.5 | 0.39 | 1922 | 0.26 | 0.36 | −0.1 | 0 | cl12_IL2R_mIL2_4GS_2_9R_N_1 |
| F3 | 7896 | 62.1 | 71.8 | 24.3 | 7.89 | 4143 | 6.95 | 8.07 | −1.12 | 0 | cl12--IL2R_mIL2_4GS_2_9R_N_1 |
| F4 | 6372 | 60.8 | 68.8 | 26.8 | 14.4 | 485 | 15.4 | 14.2 | 1.2 | 7.01 | cl23_CD8_xxx_4GS_2_9R_N_1 |
| F5 | 7758 | 63.2 | 71.5 | 24.1 | 11.7 | 6012 | 11.8 | 11.7 | 0.1 | 3.44 | cl23_CD8_xxx_4GS_2_9R_N_1 |
| F6 | 7270 | 60.4 | 68.6 | 25.7 | 26.8 | 8463 | 24.4 | 25.4 | −1 | 2.53 | cl23_CD8_xxx_4GS_2_9R_N_1 |
| F7 | 6099 | 61.9 | 68.6 | 26.2 | 11.1 | 10617 | 9.79 | 10.4 | −0.61 | 34.8 | cl23_CD8_xxx_4GS_2_9R_N_1 |
| F8 | 5686 | 60.4 | 68.6 | 25.3 | 26.9 | 9559 | 22 | 26.4 | −4.4 | 15.5 | cl23_CD8_xxx_4GS_2_9R_N_1 |
| F9 | 7674 | 62.7 | 69.9 | 25.5 | 12.3 | 5146 | 11.2 | 12.3 | −1.1 | 7.14 | cl23_CD8_xxx_4GS_2_9R_N_1 |
| G10 | 5785 | 66 | 70.8 | 25 | 4.04 | 5163 | 4.46 | 3.83 | 0.63 | 16.1 | cl23_CD8_xxx_4GS_2_9R_N_1 |
| G11 | 6783 | 61 | 71.4 | 24.1 | 4.17 | 459 | 5.24 | 4.24 | 1 | 11.5 | cl1_CD45_mSiglec_4GS_2_9R_C_1 |
| G12 | 6081 | 62.4 | 69.3 | 26.6 | 4.7 | 5359 | 4 | 5.04 | −1.04 | 57.6 | cl1_CD45_mSiglec_4GS_2_9R_C_1 |
| G13 | 4545 | 69.1 | 69.2 | 26.2 | 2.91 | 5450 | 3.44 | 2.75 | 0.69 | 6.9 | cl1_CD45_mSiglec_4GS_2_9R_C_1 |
| G14 | 5374 | 64.3 | 71.8 | 24.5 | 5.8 | 4243 | 7.94 | 5.04 | 2.9 | 23.2 | cl1_CD45_mSiglec_4GS_2_9R_C_1 |
| G15 | 5993 | 61.8 | 69.1 | 26.7 | 7.27 | 2885 | 8.3 | 6.9 | 1.4 | 41.9 | cl1_CD45_mSiglec_4GS_2_9R_C_1 |
| G16 | 4955 | 61.8 | 69.2 | 26.3 | 9.81 | 10617 | 9.78 | 9.29 | 0.49 | 44 | cl7_CD137_m41BBlg_4GS_2_9R_N_1 |
| G17 | 6054 | 58.4 | 68.7 | 26.8 | 6.61 | 1501 | 8.07 | 6.16 | 1.91 | 15.7 | cl7_CD137_m41BBlg_4GS_2_9R_N_1 |
| G18 | 5370 | 62.9 | 70.6 | 24.5 | 7.79 | 5487 | 7.95 | 7.66 | 0.29 | 71 | cl7_CD137_m41BBlg_4GS_2_9R_N_1 |
| G19 | 10120 | 62.9 | 66.6 | 28.9 | 5.32 | 3594 | 6.53 | 5.27 | 1.26 | 30.6 | cl12_IL2R_mIL2_4GS_2_9R_N_1 |
| G21 | 8544 | 67.6 | 70 | 26.7 | 1.61 | 627 | 3.01 | 0.99 | 2.02 | 1.11 | cl11--CD3_CD3e_TFA_4GS_2_9R_N_1 |
| G3 | 9912 | 59.5 | 70.6 | 25.7 | 7.28 | 5676 | 9.7 | 6.54 | 3.16 | 0.24 | cl23_CD8_xxx_4GS_2_9R_N_1 |
| G4 | 9508 | 68.7 | 69.7 | 26.5 | 2.16 | 4776 | 1.87 | 2.25 | −0.38 | 12.6 | cl23_CD8_xxx_4GS_2_9R_N_1 |
| G5 | 7916 | 66.6 | 70.2 | 25.4 | 8.25 | 5972 | 10.1 | 7.27 | 2.83 | 7.62 | cl23_CD8_xxx_4GS_2_9R_N_1 |
| G6 | 9294 | 62.4 | 69.7 | 26.2 | 8.07 | 4586 | 9.99 | 7.52 | 2.47 | 2.44 | cl23_CD8_xxx_4GS_2_9R_N_1 |
| G7 | 8845 | 67.4 | 71.5 | 25 | 6.94 | 5094 | 7.94 | 6.35 | 1.59 | 11 | cl23_CD8_xxx_4GS_2_9R_N_1 |
| G8 | 9080 | 63.3 | 69.3 | 25.9 | 14.4 | 7962 | 14.2 | 13.4 | 0.8 | 8.01 | cl23_CD8_xxx_4GS_2_9R_N_1 |
| G9 | 7716 | 56.1 | 68.7 | 25.4 | 30.3 | 505 | 30.4 | 30 | 0.4 | 3.95 | cl23_CD8_xxx_4GS_2_9R_N_1 |
| H10 | 6823 | 61.1 | 70 | 25.9 | 12.3 | 4891 | 11 | 12.5 | −1.5 | 17.5 | cl1_CD45_mSiglec_4GS_2_9R_C_1 |
| H11 | 6173 | 61.8 | 70.4 | 25.7 | 6.29 | 1778 | 7.69 | 6.01 | 1.68 | 12.9 | cl1_CD45_mSiglec_4GS_2_9R_C_1 |
| H12 | 4642 | 65.7 | 72.6 | 24.5 | 1.5 | 4974 | 1.8 | 1.5 | 0.3 | 40.9 | cl1_CD45_mSiglec_4GS_2_9R_C_1 |
| H13 | 6038 | 60.6 | 70 | 25.3 | 5.58 | 4825 | 6.17 | 5.25 | 0.92 | 32.5 | cl1_CD45_mSiglec_4GS_2_9R_C_1 |
| H14 | 5268 | 59.5 | 69.8 | 25.9 | 4.98 | 6283 | 4.91 | 5.23 | −0.32 | 37.3 | cl1_CD45_mSiglec_4GS_2_9R_C_1 |
| H15 | 5492 | 60 | 69.8 | 26.3 | 7.45 | 2268 | 8.19 | 7.18 | 1.01 | 48.9 | cl7_CD137_m41BBlg_4GS_2_9R_N_1 |
| H16 | 5079 | 61.6 | 68.3 | 27.8 | 4.61 | 9272 | 4.98 | 4.33 | 0.65 | 14.3 | cl7_CD137_m41BBlg_4GS_2_9R_N_1 |
| H17 | 6977 | 63.8 | 70.8 | 25 | 8.09 | 382 | 8.94 | 8.31 | 0.63 | 4.71 | cl12_IL2R_mIL2_4GS_2_9R_N_1 |
| H18 | 4882 | 62 | 70.1 | 25.8 | 4.74 | 8236 | 5.52 | 4.45 | 1.07 | 8.57 | cl12_IL2R_mIL2_4GS_2_9R_N_1 |
| H19 | 10326 | 70.2 | 69.3 | 26.8 | 0.45 | 355 | 0.7 | 0.36 | 0.34 | 3.23 | z_en1--_xx_nuc13 |
| H21 | 10123 | 69.1 | 69.7 | 26.7 | 1.11 | 862 | 1.95 | 0.9 | 1.05 | 1.33 | cl6--CD28_mCD86_4GS_2_9R_N_2 |
| H3 | 7680 | 68.4 | 74 | 22.7 | 3.88 | 4494 | 5.57 | 3.33 | 2.24 | 4.57 | cl23_CD8_xxx_4GS_2_9R_N_1 |
| H4 | 8044 | 58.9 | 71.7 | 24.8 | 6.95 | 4033 | 8.3 | 6.46 | 1.84 | 1.25 | cl23_CD8_xxx_4GS_2_9R_N_1 |
| H5 | 8044 | 61.1 | 70.2 | 25.8 | 6.91 | 6588 | 7.13 | 6.53 | 0.6 | 2.45 | cl23_CD8_xxx_4GS_2_9R_N_1 |
| H6 | 8264 | 61.5 | 71.2 | 25 | 6.39 | 4874 | 7.11 | 6.37 | 0.74 | 1.92 | cl23_CD8_xxx_4GS_2_9R_N_1 |
| H7 | 7248 | 67.8 | 70.5 | 25.3 | 5.21 | 4186 | 6.67 | 4.61 | 2.06 | 9.02 | cl23_CD8_xxx_4GS_2_9R_N_1 |
| H8 | 6601 | 60.8 | 71.1 | 25.2 | 7.39 | 6768 | 8.92 | 6.8 | 2.12 | 6.23 | cl23_CD8_xxx_4GS_2_9R_N_1 |
| H9 | 6995 | 63.2 | 71 | 24.9 | 11.8 | 477 | 13.7 | 12.1 | 1.6 | 2.83 | cl23_CD8_xxx_4GS_2_9R_N_1 |
| I10 | 6515 | 59.5 | 69.2 | 25.8 | 11.8 | 7776 | 11.8 | 10.7 | 1.1 | 25.8 | cl1_CD45_mSiglec_4GS_2_9R_C_1 |
| I11 | 6494 | 58.3 | 70.5 | 25.2 | 3.13 | 610 | 4.93 | 2.83 | 2.1 | 10.6 | cl1_CD45_mSiglec_4GS_2_9R_C_1 |
| I12 | 6672 | 61.7 | 71.1 | 25.2 | 4.28 | 8181 | 4.5 | 3.93 | 0.57 | 57.1 | cl1_CD45_mSiglec_4GS_2_9R_C_1 |
| I13 | 5029 | 62.3 | 70.3 | 24.8 | 8.44 | 4760 | 8.95 | 7.91 | 1.04 | 8.63 | cl1_CD45_mSiglec_4GS_2_9R_C_1 |
| I14 | 6916 | 62.2 | 70.3 | 25.7 | 4.93 | 6768 | 5.96 | 4.76 | 1.2 | 7.28 | cl7_CD137_m41BBlg_4GS_2_9R_N_1 |
| I15 | 5779 | 56.5 | 71.3 | 24.5 | 12.7 | 404 | 17.3 | 11.9 | 5.4 | 2 | cl7_CD137_m41BBlg_4GS_2_9R_N_1 |
| I16 | 7264 | 61.5 | 71.5 | 24.7 | 8.88 | 8016 | 9.35 | 8.28 | 1.07 | 46.4 | cl7_CD137_m41BBlg_4GS_2_9R_N_1 |
| I17 | 6058 | 54.3 | 68 | 26.7 | 10.8 | 475 | 12.1 | 10.6 | 1.5 | 1.47 | cl12_IL2R_mIL2_4GS_2_9R_N_1 |
| I18 | 3925 | 58.4 | 70.5 | 24.9 | 5.32 | 7882 | 6.88 | 5.18 | 1.7 | 9.32 | cl12_IL2R_mIL2_4GS_2_9R_N_1 |
| I19 | 10112 | 70.1 | 67.3 | 29 | 1.8 | 954 | 2.13 | 1.97 | 0.16 | 3.28 | zzMini_Core |
| I21 | 10095 | 72.6 | 70.2 | 26.6 | 0.31 | 1909 | 0.74 | 0.2 | 0.54 | 0 | cl2--CD28_mCD80_4GS_2_9R_N_1 |
| I3 | 6794 | 62.2 | 70.4 | 25.9 | 6.5 | 5233 | 6.88 | 6.39 | 0.49 | 8.27 | cl23_CD8_xxx_4GS_2_9R_N_1 |
| I4 | 8488 | 60 | 70.1 | 25.8 | 11.1 | 5562 | 11.5 | 10.8 | 0.7 | 2.55 | cl23_CD8_xxx_4GS_2_9R_N_1 |
| I5 | 6778 | 61.7 | 69.4 | 26.7 | 10.2 | 3440 | 10.3 | 10.3 | 0 | 13.5 | cl23_CD8_xxx_4GS_2_9R_N_1 |
| I6 | 5647 | 55.3 | 70.7 | 25 | 10.1 | 7594 | 11.3 | 9.55 | 1.75 | 5.5 | cl23_CD8_xxx_4GS_2_9R_N_1 |
| I7 | 8482 | 57.1 | 67.9 | 26.4 | 21.5 | 3359 | 22.9 | 20.9 | 2 | 18.2 | cl23_CD8_xxx_4GS_2_9R_N_1 |
| I8 | 5206 | 58.2 | 69.5 | 26.3 | 13 | 8491 | 13.7 | 12.1 | 1.6 | 18.8 | cl23_CD8_xxx_4GS_2_9R_N_1 |
| I9 | 5665 | 58.2 | 70.1 | 25.2 | 22.9 | 664 | 22.9 | 22.4 | 0.5 | 3.19 | cl23_CD8_xxx_4GS_2_9R_N_1 |
| J10 | 6531 | 64.7 | 70.8 | 25.1 | 3.3 | 6136 | 3.26 | 3.18 | 0.08 | 14.3 | cl1_CD45_mSiglec_4GS_2_9R_C_1 |
| J11 | 6758 | 58.7 | 69.5 | 26.3 | 6.4 | 8754 | 6.62 | 5.72 | 0.9 | 54.1 | cl1_CD45_mSiglec_4GS_2_9R_C_1 |
| J12 | 4102 | 69 | 72.3 | 23.9 | 7.05 | 3035 | 10 | 6.74 | 3.26 | 0 | cl1_CD45_mSiglec_4GS_2_9R_C_1 |
| J13 | 5993 | 62.1 | 70.7 | 24.9 | 3.99 | 9788 | 4.68 | 3.53 | 1.15 | 9.72 | cl1_CD45_mSiglec_4GS_2_9R_C_1 |
| J14 | 5316 | 56.3 | 68.8 | 26.6 | 5.36 | 701 | 9.4 | 4.71 | 4.69 | 23.7 | cl7_CD137_m41BBlg_4GS_2_9R_N_1 |
| J15 | 6142 | 61.7 | 70.8 | 24.7 | 6.31 | 4228 | 9.14 | 5.45 | 3.69 | 8.73 | cl7_CD137_m41BBlg_4GS_2_9R_N_1 |
| J16 | 5329 | 65.9 | 69.7 | 26.2 | 2.81 | 2268 | 3.88 | 2.79 | 1.09 | 6.38 | cl7_CD137_m41BBlg_4GS_2_9R_N_1 |
| J17 | 5798 | 71.9 | 68.7 | 26.7 | 2.63 | 4301 | 3.53 | 2.35 | 1.18 | 32.1 | cl12_IL2R_mIL2_4GS_2_9R_N_1 |
| J18 | 6476 | 60.8 | 68.5 | 27.2 | 3.04 | 1579 | 4.51 | 2.61 | 1.9 | 24.6 | cl12_IL2R_mIL2_4GS_2_9R_N_1 |
| J21 | 9768 | 64.5 | 70.3 | 26.5 | 2.64 | 1778 | 3.5 | 2.2 | 1.3 | 0 | zzzCore |
| J3 | 8504 | 67.8 | 69.5 | 27.2 | 2.13 | 5469 | 2.51 | 1.99 | 0.52 | 52.1 | cl4--CD28_mCD86_4GS_2_9R_N_1 |
| J4 | 8550 | 61 | 70.9 | 25.6 | 5.83 | 4586 | 7 | 5.53 | 1.47 | 1.02 | cl23_CD8_xxx_4GS_2_9R_N_1 |
| J5 | 8849 | 64.3 | 69.9 | 26.2 | 6.16 | 8636 | 7.24 | 5.46 | 1.78 | 16.4 | cl23_CD8_xxx_4GS_2_9R_N_1 |
| J6 | 7675 | 59 | 71.6 | 24.3 | 9.3 | 6543 | 10.9 | 8.68 | 2.22 | 2.44 | cl23_CD8_xxx_4GS_2_9R_N_1 |
| J7 | 8902 | 64.8 | 70.8 | 25.4 | 6.68 | 5793 | 6.49 | 6.5 | −0.01 | 14.6 | cl23_CD8_xxx_4GS_2_9R_N_1 |
| J8 | 6315 | 61.6 | 70.8 | 25.3 | 11.4 | 6219 | 11.9 | 11 | 0.9 | 3.25 | cl23_CD8_xxx_4GS_2_9R_N_1 |
| J9 | 7233 | 58.5 | 68.9 | 25.6 | 14.6 | 7121 | 15.4 | 13.8 | 1.6 | 9.53 | cl23_CD8_xxx_4GS_2_9R_N_1 |
| K10 | 3979 | 59 | 70.1 | 25 | 11 | 9989 | 10.3 | 10.2 | 0.1 | 32.4 | cl23_CD8_xxx_4GS_2_9R_N_1 |
| K11 | 7146 | 61.9 | 72.1 | 24.2 | 4.37 | 6433 | 4.63 | 4.44 | 0.19 | 11.2 | cl1_CD45_mSiglec_4GS_2_9R_C_1 |
| K12 | 4589 | 57.7 | 69.6 | 26.2 | 6.36 | 8181 | 5.61 | 6.07 | −0.46 | 5.49 | cl1_CD45_mSiglec_4GS_2_9R_C_1 |
| K13 | 5021 | 62.2 | 71.3 | 24.5 | 5.25 | 7594 | 4.88 | 4.94 | −0.06 | 9.49 | cl1_CD45_mSiglec_4GS_2_9R_C_1 |
| K14 | 2756 | 64.4 | 69.7 | 26.6 | 1.96 | 6219 | 3.25 | 1.57 | 1.68 | 29.4 | cl1_CD45_mSiglec_4GS_2_9R_C_1 |
| K15 | 7381 | 60.2 | 69.8 | 26.2 | 6.49 | 5146 | 6.68 | 6.49 | 0.19 | 10.8 | cl7_CD137_m41BBlg_4GS_2_9R_N_1 |
| K16 | 3245 | 59.4 | 69.3 | 26.3 | 6.01 | 5506 | 5.6 | 6.16 | −0.56 | 35.1 | cl7_CD137_m41BBlg_4GS_2_9R_N_1 |
| K17 | 7166 | 63.1 | 69 | 26.6 | 6.42 | 2554 | 7.04 | 6.42 | 0.62 | 14.6 | cl7_CD137_m41BBlg_4GS_2_9R_N_1 |
| K18 | 5137 | 61.3 | 70.7 | 25.2 | 6.46 | 6115 | 6.49 | 6.12 | 0.37 | 4.06 | cl12_IL2R_mIL2_4GS_2_9R_N_1 |
| K21 | 6333 | 77.1 | 74.1 | 22.7 | 3.04 | 1408 | 4.03 | 2.87 | 1.16 | 0 | Lipo2 |
| K3 | 10077 | 49.9 | 70 | 25.2 | 1.91 | 2723 | 2.42 | 1.92 | 0.5 | 0 | cl14--ESELlg_mESEL_4GS2_9R_N_1 |
| K4 | 6052 | 57.3 | 72.2 | 24.5 | 6.12 | 3440 | 8.01 | 5.64 | 2.37 | 0 | cl23_CD8_xxx_4GS_2_9R_N_1 |
| K5 | 7742 | 61 | 69.9 | 26 | 4.88 | 3846 | 4.44 | 5.08 | −0.64 | 6.36 | cl23_CD8_xxx_4GS_2_9R_N_1 |
| K6 | 6243 | 57.7 | 71.3 | 25.3 | 14.1 | 4602 | 13.5 | 14.5 | −1 | 8.55 | cl23_CD8_xxx_4GS_2_9R_N_1 |
| K7 | 9506 | 61.8 | 70 | 26.1 | 9.32 | 5715 | 8.79 | 9.64 | −0.85 | 34.4 | cl23_CD8_xxx_4GS_2_9R_N_1 |
| K8 | 4234 | 56.9 | 70 | 25.9 | 10.3 | 8292 | 10.4 | 9.46 | 0.94 | 5.83 | cl23_CD8_xxx_4GS_2_9R_N_1 |
| K9 | 5564 | 57.9 | 69.1 | 26.6 | 16.5 | 5715 | 15.8 | 16.4 | −0.6 | 11.8 | cl23_CD8_xxx_4GS_2_9R_N_1 |
| L10 | 6274 | 57.4 | 71 | 25 | 17.3 | 347 | 20.4 | 17.4 | 3 | 11 | cl23_CD8_xxx_4GS_2_9R_N_1 |
| L11 | 5618 | 67.1 | 69.5 | 26.7 | 1.94 | 7391 | 2.98 | 1.42 | 1.56 | 32.4 | cl1_CD45_mSiglec_4GS_2_9R_C_1 |
| L12 | 5858 | 61.2 | 71.9 | 24.2 | 5.65 | 6861 | 6.55 | 5.33 | 1.22 | 9.69 | cl1_CD45_mSiglec_4GS_2_9R_C_1 |
| L13 | 6013 | 59.6 | 69 | 26.4 | 7.34 | 8873 | 5.31 | 7.69 | −2.38 | 40.2 | cl1_CD45_mSiglec_4GS_2_9R_C_1 |
| L14 | 4951 | 60.6 | 70.4 | 24.7 | 7.47 | 10298 | 7.35 | 6.72 | 0.63 | 57.8 | cl1_CD45_mSiglec_4GS_2_9R_C_1 |
| L15 | 5251 | 64.4 | 69.7 | 26.7 | 8.94 | 8126 | 7.51 | 8.94 | −1.43 | 18.4 | cl7_CD137_m41BBlg_4GS_2_9R_N_1 |
| L16 | 4836 | 61.3 | 70.6 | 25 | 6.68 | 6838 | 7.06 | 6.47 | 0.59 | 1.04 | cl7_CD137_m41BBlg_4GS_2_9R_N_1 |
| L17 | 6467 | 64.6 | 71.9 | 23.9 | 3.13 | 10368 | 3.63 | 2.86 | 0.77 | 36.5 | cl7_CD137_m41BBlg_4GS_2_9R_N_1 |
| L18 | 6010 | 62.7 | 72.6 | 23.2 | 5.58 | 8292 | 5.52 | 5.7 | −0.18 | 7.32 | cl12_IL2R_mIL2_4GS_2_9R_N_1 |
| L21 | 10193 | 67.5 | 70.6 | 26.5 | 0.015 | 334 | 0.057 | 0 | 0.057 | 0 | No TF 1 |
| L3 | 9997 | 62.5 | 71.8 | 25.2 | 0.78 | 2984 | 1.26 | 0.65 | 0.61 | 2.13 | cl9--CD3_mCD3Ab_4GS_2_9R_N_1 |
| L4 | 9882 | 53.2 | 71.7 | 24 | 12.1 | 354 | 14.9 | 11.9 | 3 | 3.47 | cl23_CD8_xxx_4GS_2_9R_N_1 |
| L5 | 8849 | 59.3 | 71.1 | 25.1 | 7.58 | 2678 | 8.46 | 7.78 | 0.68 | 4.16 | cl23_CD8_xxx_4GS_2_9R_N_1 |
| L6 | 7137 | 58.4 | 72.6 | 23.6 | 10.6 | 394 | 13.5 | 10.2 | 3.3 | 2.17 | cl23_CD8_xxx_4GS_2_9R_N_1 |
| L7 | 6679 | 62.4 | 70.6 | 25.3 | 3.26 | 3718 | 4.2 | 2.87 | 1.33 | 25.6 | cl23_CD8_xxx_4GS_2_9R_N_1 |
| L8 | 7631 | 55.8 | 70.3 | 25.1 | 14.9 | 402 | 16.7 | 15.2 | 1.5 | 5.08 | cl23_CD8_xxx_4GS_2_9R_N_1 |
| L9 | 6597 | 57.8 | 71 | 24.8 | 15 | 1302 | 17 | 13.5 | 3.5 | 9.89 | cl23_CD8_xxx_4GS_2_9R_N_1 |
| M10 | 8737 | 55.6 | 70.7 | 25.7 | 3.31 | 4200 | 4.02 | 3.16 | 0.86 | 9.55 | cl1_CD45_mSiglec_4GS_2_9R_C_1 |
| M11 | 7089 | 62.6 | 70.9 | 25.9 | 1.49 | 4825 | 2.26 | 1.28 | 0.98 | 37.5 | cl1_CD45_mSiglec_4GS_2_9R_C_1 |
| M12 | 7809 | 59.1 | 71.5 | 24.6 | 5.61 | 5657 | 5.17 | 5.75 | −0.58 | 26.3 | cl1_CD45_mSiglec_4GS_2_9R_C_1 |
| M13 | 7307 | 61.2 | 72.1 | 24.3 | 4.2 | 3794 | 4.55 | 4.06 | 0.49 | 0 | cl1_CD45_mSiglec_4GS_2_9R_C_1 |
| M14 | 8185 | 68.8 | 70.5 | 25.7 | 1.61 | 5025 | 2 | 1.38 | 0.62 | 31.8 | cl1_CD45_mSiglec_4GS_2_9R_C_1 |
| M15 | 6967 | 58.7 | 69.9 | 26.1 | 6.94 | 6198 | 7.06 | 6.89 | 0.17 | 8 | cl7_CD137_m41BBlg_4GS_2_9R_N_1 |
| M16 | 5910 | 57.2 | 70.7 | 25.2 | 3.64 | 5216 | 4.09 | 3.74 | 0.35 | 0.83 | cl7_CD137_m41BBlg_4GS_2_9R_N_1 |
| M17 | 7427 | 59.6 | 68.7 | 26.9 | 2.63 | 1259 | 3.52 | 2.55 | 0.97 | 19.8 | cl7_CD137_m41BBlg_4GS_2_9R_N_1 |
| M18 | 6921 | 62.1 | 68.7 | 26.6 | 7.36 | 7242 | 7.99 | 6.43 | 1.56 | 30.5 | cl12_IL2R_mIL2_4GS_2_9R_N_1 |
| M21 | 10104 | 62.3 | 72.7 | 24.1 | 0.049 | 256 | 0.13 | 0 | 0.13 | 33.3 | No TF 2 |
| M3 | 7991 | 68.3 | 71.8 | 24.6 | 2.59 | 2387 | 3.34 | 2.59 | 0.75 | 0 | cl23_CD8_xxx_4GS_2_9R_N_1 |
| M4 | 9963 | 66.9 | 70.2 | 26.3 | 2.35 | 2733 | 3 | 2.11 | 0.89 | 0.67 | cl23_CD8_xxx_4GS_2_9R_N_1 |
| M5 | 8871 | 49.2 | 72.8 | 23.9 | 3.34 | 3405 | 4.13 | 3.03 | 1.1 | 0.7 | cl23_CD8_xxx_4GS_2_9R_N_1 |
| M6 | 10049 | 60.8 | 69.8 | 26.6 | 5.31 | 2511 | 6.49 | 4.97 | 1.52 | 0.96 | cl23_CD8_xxx_4GS_2_9R_N_1 |
| M7 | 7968 | 57.9 | 70.3 | 25.8 | 6.85 | 3171 | 8.33 | 6.78 | 1.55 | 13.1 | cl23_CD8_xxx_4GS_2_9R_N_1 |
| M8 | 9780 | 60.8 | 69.8 | 25.4 | 15.4 | 7169 | 15.7 | 14.4 | 1.3 | 4.67 | cl23_CD8_xxx_4GS_2_9R_N_1 |
| M9 | 7011 | 55.2 | 72.2 | 23.8 | 10.1 | 2171 | 12.4 | 9.66 | 2.74 | 3.99 | cl23_CD8_xxx_4GS_2_9R_N_1 |
| N10 | 7288 | 64.9 | 71.8 | 24.3 | 3 | 6177 | 4.3 | 2.6 | 1.7 | 8.89 | cl1_CD45_mSiglec_4GS_2_9R_C_1 |
| N11 | 7878 | 60.5 | 71.3 | 25.1 | 5.55 | 6240 | 5.72 | 5.38 | 0.34 | 0.39 | cl1_CD45_mSiglec_4GS_2_9R_C_1 |
| N12 | 7202 | 62.5 | 72.8 | 23.8 | 1.91 | 1790 | 2.25 | 1.73 | 0.52 | 8.54 | cl1_CD45_mSiglec_4GS_2_9R_C_1 |
| N13 | 6339 | 61 | 70.3 | 25.6 | 3.96 | 5323 | 5.2 | 3.67 | 1.53 | 17.4 | cl1_CD45_mSiglec_4GS_2_9R_C_1 |
| N14 | 6387 | 61.5 | 70.4 | 25.9 | 2.76 | 5715 | 3.31 | 2.7 | 0.61 | 31.1 | cl1_CD45_mSiglec_4GS_2_9R_C_1 |
| N15 | 6355 | 60.7 | 71.2 | 24.6 | 4.92 | 10474 | 5.34 | 4.54 | 0.8 | 8.7 | cl7_CD137_m41BBlg_4GS_2_9R_N_1 |
| N16 | 5455 | 50.3 | 69.5 | 26.2 | 7.37 | 6907 | 7.23 | 7.39 | −0.16 | 26.2 | cl7_CD137_m41BBlg_4GS_2_9R_N_1 |
| N17 | 5772 | 65.2 | 69.7 | 26.2 | 2.17 | 7049 | 3.36 | 1.77 | 1.59 | 38 | cl7_CD137_m41BBlg_4GS_2_9R_N_1 |
| N18 | 5977 | 61.1 | 70.5 | 25 | 6.71 | 454 | 9.13 | 6.36 | 2.77 | 2.59 | cl12_IL2R_mIL2_4GS_2_9R_N_1 |
| N3 | 9942 | 58.4 | 76.3 | 20.7 | 4.14 | 1673 | 4.58 | 4.13 | 0.45 | 0.87 | cl23_CD8_xxx_4GS_2_9R_N_1 |
| N4 | 10007 | 60 | 72.7 | 23.6 | 4.57 | 3643 | 6.01 | 4.29 | 1.72 | 1.53 | cl23_CD8_xxx_4GS_2_9R_N_1 |
| N5 | 7519 | 44.8 | 75.5 | 20.5 | 5.5 | 4046 | 6.87 | 5.5 | 1.37 | 6.11 | cl23_CD8_xxx_4GS_2_9R_N_1 |
| N6 | 8587 | 54 | 69.3 | 26.8 | 7.26 | 1701 | 9.1 | 6.91 | 2.19 | 8.02 | cl23_CD8_xxx_4GS_2_9R_N_1 |
| N7 | 7603 | 55.9 | 71.8 | 23.8 | 7.81 | 6156 | 9.16 | 7.25 | 1.91 | 4.02 | cl23_CD8_xxx_4GS_2_9R_N_1 |
| N8 | 8843 | 54.7 | 70.6 | 25.4 | 7.04 | 1784 | 8.06 | 7.11 | 0.95 | 5.5 | cl23_CD8_xxx_4GS_2_9R_N_1 |
| N9 | 7642 | 57.9 | 69.9 | 25.9 | 12.1 | 5251 | 11.3 | 12.4 | −1.1 | 2.5 | cl23_CD8_xxx_4GS_2_9R_N_1 |

| Cell | Ligand_2 | Ligand_3 | Ligand_4 | ratio |
|---|---|---|---|---|
| C10 | cl6_CD28_mCD86_4GS_2_9R_N_2 | cl2_CD28_mCD80_4GS_2_9R_N_1 | cl4_CD28_mCD86_4GS_2_9R_N_1 | 25-25-25-25 |
| C11 | cl7_CD137_m41BBlg_4GS_2_9R_N_1 | cl12_IL2R_mIL2_4GS_2_9R_N_1 | cl2_CD28_mCD80_4GS_2_9R_N_1 | 25-25-25-25 |
| C12 | cl7_CD137_m41BBlg_4GS_2_9R_N_1 | cl6_CD28_mCD86_4GS_2_9R_N_2 | cl9_CD3_mCD3Ab_4GS_2_9R_N_1 | 25-25-25-25 |
| C13 | cl12_IL2R_mIL2_4GS_2_9R_N_1 | cl6_CD28_mCD86_4GS_2_9R_N_2 | cl2_CD28_mCD80_4GS_2_9R_N_1 | 25-25-25-25 |
| C14 | cl11_CD3_CD3e_TFA_4GS_2_9R_N_1 | cl6_CD28_mCD86_4GS_2_9R_N_2 | cl14_ESELlg_mESEL_4GS_2_9R_N_1 | 25-25-25-25 |
| C15 | cl6_CD28_mCD86_4GS_2_9R_N_2 | cl4_CD28_mCD86_4GS_2_9R_N_1 | cl9_CD3_mCD3Ab_4GS_2_9R_N_1 | 25-25-25-25 |
| C16 | cl12_IL2R_mIL2_4GS_2_9R_N_1 | cl6_CD28_mCD86_4GS_2_9R_N_2 | cl4_CD28_mCD86_4GS_2_9R_N_1 | 25-25-25-25 |
| C17 | cl11_CD3_CD3e_TFA_4GS_2_9R_N_1 | cl6_CD28_mCD86_4GS_2_9R_N_2 | cl9_CD3_mCD3Ab_4GS_2_9R_N_1 | 25-25-25-25 |
| C18 | cl6_CD28_mCD86_4GS_2_9R_N_2 | cl14_ESELlg_mESEL_4GS_2_9R_N_1 | cl9_CD3_mCD3Ab_4GS_2_9R_N_1 | 25-25-25-25 |
| C19 | cl11_CD3_CD3e_TFA_4GS_2_9R_N_1 | cl4_CD28_mCD86_4GS_2_9R_N_1 | cl14_ESELlg_mESEL_4GS_2_9R_N_1 | 25-25-25-25 |
| C3 | | | | |
| C4 | cl1_CD45_mSiglec_4GS_2_9R_C_1 | cl7_CD137_m41BBlg_4GS_2_9R_N_1 | cl6_CD28_mCD86_4GS_2_9R_N_2 | 25-25-25-25 |
| C5 | cl1_CD45_mSiglec_4GS_2_9R_C_1 | cl11_CD3_CD3e_TFA_4GS_2_9R_N_1 | cl2_CD28_mCD80_4GS_2_9R_N_1 | 25-25-25-25 |
| C6 | cl1_CD45_mSiglec_4GS_2_9R_C_1 | cl4_CD28_mCD86_4GS_2_9R_N_1 | cl9_CD3_mCD3Ab_4GS_2_9R_N_1 | 25-25-25-25 |
| C7 | cl7_CD137_m41BBlg_4GS_2_9R_N_1 | cl11_CD3_CD3c_TFA_4GS_2_9R_N_1 | cl9_CD3_mCD3Ab_4GS_2_9R_N_1 | 25-25-25-25 |
| C8 | cl12_IL2R_mIL2_4GS_2_9R_N_1 | cl11_CD3_CD3e_TFA_4GS_2_9R_N_1 | cl2_CD28_mCD80_4GS_2_9R_N_1 | 25-25-25-25 |
| C9 | cl12_IL2R_mIL2_4GS_2_9R_N_1 | cl4_CD28_mCD86_4GS_2_9R_N_1 | cl9_CD3_mCD3Ab_4GS_2_9R_N_1 | 25-25-25-25 |
| D10 | cl6_CD28_mCD86_4GS_2_9R_N_2 | cl2_CD28_mCD80_4GS_2_9R_N_1 | cl14_ESELlg_mESEL_4GS_2_9R_N_1 | 25-25-25-25 |
| D11 | cl7_CD137_m41BBlg_4GS_2_9R_N_1 | cl12_IL2R_mIL2_4GS_2_9R_N_1 | cl4_CD28_mCD86_4GS_2_9R_N_1 | 25-25-25-25 |
| D12 | cl7_CD137_m41BBlg_4GS_2_9R_N_1 | cl2_CD28_mCD80_4GS_2_9R_N_1 | cl4_CD28_mCD86_4GS_2_9R_N_1 | 25-25-25-25 |
| D13 | cl12_IL2R_mIL2_4GS_2_9R_N_1 | cl6_CD28_mCD86_4GS_2_9R_N_2 | cl4_CD28_mCD86_4GS_2_9R_N_1 | 25-25-25-25 |
| D14 | cl11_CD3_CD3e_TFA_4GS_2_9R_N_1 | cl6_CD28_mCD86_4GS_2_9R_N_2 | cl9_CD3_mCD3Ab_4GS_2_9R_N_1 | 25-25-25-25 |
| D15 | cl6_CD28_mCD86_4GS_2_9R_N_2 | cl14_ESELlg_mESEL_4GS_2_9R_N_1 | cl9_CD3_mCD3Ab_4GS_2_9R_N_1 | 25-25-25-25 |
| D16 | cl12_IL2R_mIL2_4GS_2_9R_N_1 | cl6_CD28_mCD86_4GS_2_9R_N_2 | cl14_ESELlg_mESEL_4GS_2_9R_N_1 | 25-25-25-25 |
| D17 | cl11_CD3_CD3e_TFA_4GS_2_9R_N_1 | cl2_CD28_mCD80_4GS_2_9R_N_1 | cl4_CD28_mCD86_4GS_2_9R_N_1 | 25-25-25-25 |
| D18 | cl2_CD28_mCD80_4GS_2_9R_N_1 | cl4_CD28_mCD86_4GS_2_9R_N_1 | cl14_ESELlg_mESEL_4GS_2_9R_N_1 | 25-25-25-25 |
| D19 | cl11_CD3_CD3e_TFA_4GS_2_9R_N_1 | cl4_CD28_mCD86_4GS_2_9R_N_1 | cl9_CD3_mCD3Ab_4GS_2_9R_N_1 | 25-25-25-25 |
| D3 | | | | |
| D4 | cl1_CD45_mSiglec_4GS_2_9R_C_1 | cl7_CD137_m41BBlg_4GS_2_9R_N_1 | cl2_CD28_mCD80_4GS_2_9R_N_1 | 25-25-25-25 |
| D5 | cl1_CD45_mSiglec_4GS_2_9R_C_1 | cl11_CD3_CD3e_TFA_4GS_2_9R_N_1 | cl4_CD28_mCD86_4GS_2_9R_N_1 | 25-25-25-25 |
| D6 | cl1_CD45_mSiglec_4GS_2_9R_C_1 | cl14_ESELlg_mESEL_4GS_2_9R_N_1 | cl9_CD3_mCD3Ab_4GS_2_9R_N_1 | 25-25-25-25 |
| D7 | cl7_CD137_m41BBlg_4GS_2_9R_N_1 | cl6_CD28_mCD86_4GS_2_9R_N_2 | cl2_CD28_mCD80_4GS_2_9R_N_1 | 25-25-25-25 |
| D8 | cl12_IL2R_mIL2_4GS_2_9R_N_1 | cl11_CD3_CD3e_TFA_4GS_2_9R_N_1 | cl4_CD28_mCD86_4GS_2_9R_N_1 | 25-25-25-25 |
| D9 | cl12_IL2R_mIL2_4GS_2_9R_N_1 | cl14_ESELlg_mESEL_4GS_2_9R_N_1 | cl9_CD3_mCD3Ab_4GS_2_9R_N_1 | 25-25-25-25 |
| E10 | cl6_CD28_mCD86_4GS_2_9R_N_2 | cl2_CD28_mCD80_4GS_2_9R_N_1 | cl9_CD3_mCD3Ab_4GS_2_9R_N_1 | 25-25-25-25 |
| E11 | cl7_CD137_m41BBlg_4GS_2_9R_N_1 | cl12_IL2R_mIL2_4GS_2_9R_N_1 | cl14_ESELlg_mESEL_4GS_2_9R_N_1 | 25-25-25-25 |
| E12 | cl7_CD137_m41BBlg_4GS_2_9R_N_1 | cl2_CD28_mCD80_4GS_2_9R_N_1 | cl14_ESELlg_mESEL_4GS_2_9R_N_1 | 25-25-25-25 |
| E13 | cl12_IL2R_mIL2_4GS_2_9R_N_1 | cl6_CD28_mCD86_4GS_2_9R_N_2 | cl14_ESELlg_mESEL_4GS_2_9R_N_1 | 25-25-25-25 |
| E14 | cl11_CD3_CD3e_TFA_4GS_2_9R_N_1 | cl2_CD28_mCD80_4GS_2_9R_N_1 | cl4_CD28_mCD86_4GS_2_9R_N_1 | 25-25-25-25 |
| E15 | cl2_CD28_mCD80_4GS_2_9R_N_1 | cl4_CD28_mCD86_4GS_2_9R_N_1 | cl14_ESELlg_mESEL_4GS_2_9R_N_1 | 25-25-25-25 |
| E16 | cl12_IL2R_mIL2_4GS_2_9R_N_1 | cl6_CD28_mCD86_4GS_2_9R_N_2 | cl9_CD3_mCD3Ab_4GS_2_9R_N_1 | 25-25-25-25 |
| E17 | cl11_CD3_CD3e_TFA_4GS_2_9R_N_1 | cl2_CD28_mCD80_4GS_2_9R_N_1 | cl14_ESELlg_mESEL_4GS_2_9R_N_1 | 25-25-25-25 |
| E18 | cl2_CD28_mCD80_4GS_2_9R_N_1 | cl4_CD28_mCD86_4GS_2_9R_N_1 | cl9_CD3_mCD3Ab_4GS_2_9R_N_1 | 25-25-25-25 |
| E19 | cl11_CD3_CD3e_TFA_4GS_2_9R_N_1 | cl14_ESELlg_mESEL_4GS_2_9R_N_1 | cl9_CD3_mCD3Ab_4GS_2_9R_N_1 | 25-25-25-25 |
| E3 | | | | |
| E4 | cl1_CD45_mSiglec_4GS_2_9R_C_1 | cl7_CD137_m41BBlg_4GS_2_9R_N_1 | cl4_CD28_mCD86_4GS_2_9R_N_1 | 25-25-25-25 |
| E5 | cl1_CD45_mSiglec_4GS_2_9R_C_1 | cl11_CD3_CD3e_TFA_4GS_2_9R_N_1 | cl14_ESELlg_mESEL_4GS_2_9R_N_1 | 25-25-25-25 |
| E6 | cl7_CD137_m41BBlg_4GS_2_9R_N_1 | cl12_IL2R_mIL2_4GS_2_9R_N_1 | cl11_CD3_CD3e_TFA_4GS_2_9R_N_1 | 25-25-25-25 |
| E7 | cl7_CD137_m41BBlg_4GS_2_9R_N_1 | cl6_CD28_mCD86_4GS_2_9R_N_2 | cl4_CD28_mCD86_4GS_2_9R_N_1 | 25-25-25-25 |
| E8 | cl12_IL2R_mIL2_4GS_2_9R_N_1 | cl11_CD3_CD3e_TFA_4GS_2_9R_N_1 | cl14_ESELlg_mESEL_4GS_2_9R_N_1 | 25-25-25-25 |
| E9 | cl11_CD3_CD3e_TFA_4GS_2_9R_N_1 | cl6_CD28_mCD86_4GS_2_9R_N_2 | cl2_CD28_mCD80_4GS_2_9R_N_1 | 25-25-25-25 |
| F10 | cl6_CD28_mCD86_4GS_2_9R_N_2 | cl4_CD28_mCD86_4GS_2_9R_N_1 | cl14_ESELlg_mESEL_4GS_2_9R_N_1 | 25-25-25-25 |
| F11 | cl7_CD137_m41BBlg_4GS_2_9R_N_1 | cl12_IL2R_mIL2_4GS_2_9R_N_1 | cl9_CD3_mCD3Ab_4GS_2_9R_N_1 | 25-25-25-25 |
| F12 | cl7_CD137_m41BBlg_4GS_2_9R_N_1 | cl2_CD28_mCD80_4GS_2_9R_N_1 | cl9_CD3_mCD3Ab_4GS_2_9R_N_1 | 25-25-25-25 |
| F13 | cl12_IL2R_mIL2_4GS_2_9R_N_1 | cl6_CD28_mCD86_4GS_2_9R_N_2 | cl9_CD3_mCD3Ab_4GS_2_9R_N_1 | 25-25-25-25 |
| F14 | cl11_CD3_CD3e_TFA_4GS_2_9R_N_1 | cl2_CD28_mCD80_4GS_2_9R_N_1 | cl14_ESELlg_mESEL_4GS_2_9R_N_1 | 25-25-25-25 |
| F15 | cl2_CD28_mCD80_4GS_2_9R_N_1 | cl4_CD28_mCD86_4GS_2_9R_N_1 | cl9_CD3_mCD3Ab_4GS_2_9R_N_1 | 25-25-25-25 |
| F16 | cl12_IL2R_mIL2_4GS_2_9R_N_1 | cl2_CD28_mCD80_4GS_2_9R_N_1 | cl4_CD28_mCD86_4GS_2_9R_N_1 | 25-25-25-25 |
| F17 | cl11_CD3_CD3e_TFA_4GS_2_9R_N_1 | cl2_CD28_mCD80_4GS_2_9R_N_1 | cl9_CD3_mCD3Ab_4GS_2_9R_N_1 | 25-25-25-25 |
| F18 | cl2_CD28_mCD80_4GS_2_9R_N_1 | cl14_ESELlg_mESEL_4GS_2_9R_N_1 | cl9_CD3_mCD3Ab_4GS_2_9R_N_1 | 25-25-25-25 |
| F19 | cl6_CD28_mCD86_4GS_2_9R_N_2 | cl2_CD28_mCD80_4GS_2_9R_N_1 | cl4_CD28_mCD86_4GS_2_9R_N_1 | 25-25-25-25 |
| F3 | | | | |
| F4 | cl1_CD45_mSiglec_4GS_2_9R_C_1 | cl7_CD137_m41BBlg_4GS_2_9R_N_1 | cl14_ESELlg_mESEL_4GS_2_9R_N_1 | 25-25-25-25 |
| F5 | cl1_CD45_mSiglec_4GS_2_9R_C_1 | cl11_CD3_CD3e_TFA_4GS_2_9R_N_1 | cl9_CD3_mCD3Ab_4GS_2_9R_N_1 | 25-25-25-25 |
| F6 | cl7_CD137_m41BBlg_4GS_2_9R_N_1 | cl12_IL2R_mIL2_4GS_2_9R_N_1 | cl6_CD28_mCD86_4GS_2_9R_N_2 | 25-25-25-25 |
| F7 | cl7_CD137_m41BBlg_4GS_2_9R_N_1 | cl6_CD28_mCD86_4GS_2_9R_N_2 | cl14_ESELlg_mESEL_4GS_2_9R_N_1 | 25-25-25-25 |
| F8 | cl12_IL2R_mIL2_4GS_2_9R_N_1 | cl11_CD3_CD3e_TFA_4GS_2_9R_N_1 | cl9_CD3_mCD3Ab_4GS_2_9R_N_1 | 25-25-25-25 |
| F9 | cl11_CD3_CD3e_TFA_4GS_2_9R_N_1 | cl6_CD28_mCD86_4GS_2_9R_N_2 | cl4_CD28_mCD86_4GS_2_9R_N_1 | 25-25-25-25 |
| G10 | cl6_CD28_mCD86_4GS_2_9R_N_2 | cl4_CD28_mCD86_4GS_2_9R_N_1 | cl9_CD3_mCD3Ab_4GS_2_9R_N_1 | 25-25-25-25 |
| G11 | cl7_CD137_m41BBlg_4GS_2_9R_N_1 | cl11_CD3_CD3e_TFA_4GS_2_9R_N_1 | cl6_CD28_mCD86_4GS_2_9R_N_2 | 25-25-25-25 |
| G12 | cl7_CD137_m41BBlg_4GS_2_9R_N_1 | cl4_CD28_mCD86_4GS_2_9R_N_1 | cl14_ESELlg_mESEL_4GS_2_9R_N_1 | 25-25-25-25 |
| G13 | cl12_IL2R_mIL2_4GS_2_9R_N_1 | cl2_CD28_mCD80_4GS_2_9R_N_1 | cl4_CD28_mCD86_4GS_2_9R_N_1 | 25-25-25-25 |
| G14 | cl11_CD3_CD3e_TFA_4GS_2_9R_N_1 | cl2_CD28_mCD80_4GS_2_9R_N_1 | cl9_CD3_mCD3Ab_4GS_2_9R_N_1 | 25-25-25-25 |
| G15 | cl2_CD28_mCD80_4GS_2_9R_N_1 | cl14_ESELlg_mESEL_4GS_2_9R_N_1 | cl9_CD3_mCD3Ab_4GS_2_9R_N_1 | 25-25-25-25 |
| G16 | cl12_IL2R_mIL2_4GS_2_9R_N_1 | cl2_CD28_mCD80_4GS_2_9R_N_1 | cl14_ESELlg_mESEL_4GS_2_9R_N_1 | 25-25-25-25 |
| G17 | cl11_CD3_CD3e_TFA_4GS_2_9R_N_1 | cl4_CD28_mCD86_4GS_2_9R_N_1 | cl14_ESELlg_mESEL_4GS_2_9R_N_1 | 25-25-25-25 |
| G18 | cl4_CD28_mCD86_4GS_2_9R_N_1 | cl14_ESELlg_mESEL_4GS_2_9R_N_1 | cl9_CD3_mCD3Ab_4GS_2_9R_N_1 | 25-25-25-25 |
| G19 | cl6_CD28_mCD86_4GS_2_9R_N_2 | cl2_CD28_mCD80_4GS_2_9R_N_1 | cl14_ESELlg_mESEL_4GS_2_9R_N_1 | 25-25-25-25 |
| G21 | | | | |
| G3 | cl1_CD45_mSiglec_4GS_2_9R_C_1 | cl7_CD137_m41BBlg_4GS_2_9R_N_1 | cl9_CD3_mCD3Ab_4GS_2_9R_N_1 | 25-25-25-25 |
| G4 | cl1_CD45_mSiglec_4GS_2_9R_C_1 | cl6_CD28_mCD86_4GS_2_9R_N_2 | cl2_CD28_mCD80_4GS_2_9R_N_1 | 25-25-25-25 |
| G5 | cl7_CD137_m41BBlg_4GS_2_9R_N_1 | cl12_IL2R_mIL2_4GS_2_9R_N_1 | cl2_CD28_mCD80_4GS_2_9R_N_1 | 25-25-25-25 |
| G6 | cl7_CD137_m41BBlg_4GS_2_9R_N_1 | cl6_CD28_mCD86_4GS_2_9R_N_2 | cl9_CD3_mCD3Ab_4GS_2_9R_N_1 | 25-25-25-25 |
| G7 | cl12_IL2R_mIL2_4GS_2_9R_N_1 | cl6_CD28_mCD86_4GS_2_9R_N_2 | cl2_CD28_mCD80_4GS_2_9R_N_1 | 25-25-25-25 |
| G8 | cl11_CD3_CD3c_TFA_4GS_2_9R_N_1 | cl6_CD28_mCD86_4GS_2_9R_N_2 | cl14_ESELlg_mESEL_4GS_2_9R_N_1 | 25-25-25-25 |
| G9 | cl6_CD28_mCD86_4GS_2_9R_N_2 | cl14_ESELlg_mESEL_4GS_2_9R_N_1 | cl9_CD3_mCD3Ab_4GS_2_9R_N_1 | 25-25-25-25 |
| H10 | cl7_CD137_m41BBlg_4GS_2_9R_N_1 | cl11_CD3_CD3e_TFA_4GS_2_9R_N_1 | cl2_CD28_mCD80_4GS_2_9R_N_1 | 25-25-25-25 |
| H11 | cl7_CD137_m41BBlg_4GS_2_9R_N_1 | cl4_CD28_mCD86_4GS_2_9R_N_1 | cl9_CD3_mCD3Ab_4GS_2_9R_N_1 | 25-25-25-25 |
| H12 | cl12_IL2R_mIL2_4GS_2_9R_N_1 | cl2_CD28_mCD80_4GS_2_9R_N_1 | cl14_ESELlg_mESEL_4GS_2_9R_N_1 | 25-25-25-25 |
| H13 | cl11_CD3_CD3c_TFA_4GS_2_9R_N_1 | cl4_CD28_mCD86_4GS_2_9R_N_1 | cl14_ESELlg_mESEL_4GS_2_9R_N_1 | 25-25-25-25 |
| H14 | cl4_CD28_mCD86_4GS_2_9R_N_1 | cl14_ESELlg_mESEL_4GS_2_9R_N_1 | cl9_CD3_mCD3Ab_4GS_2_9R_N_1 | 25-25-25-25 |
| H15 | cl12_IL2R_mIL2_4GS_2_9R_N_1 | cl2_CD28_mCD80_4GS_2_9R_N_1 | cl9_CD3_mCD3Ab_4GS_2_9R_N_1 | 25-25-25-25 |
| H16 | cl11_CD3_CD3e_TFA_4GS_2_9R_N_1 | cl4_CD28_mCD86_4GS_2_9R_N_1 | cl9_CD3_mCD3Ab_4GS_2_9R_N_1 | 25-25-25-25 |
| H17 | cl11_CD3_CD3e_TFA_4GS_2_9R_N_1 | cl6_CD28_mCD86_4GS_2_9R_N_2 | cl2_CD28_mCD80_4GS_2_9R_N_1 | 25-25-25-25 |
| H18 | cl6_CD28_mCD86_4GS_2_9R_N_2 | cl2_CD28_mCD80_4GS_2_9R_N_1 | cl9_CD3_mCD3Ab_4GS_2_9R_N_1 | 25-25-25-25 |
| H19 | | | | |
| H21 | | | | |
| H3 | cl1_CD45_mSiglec_4GS_2_9R_C_1 | cl12_IL2R_mIL2_4GS_2_9R_N_1 | cl11_CD3_CD3e_TFA_4GS_2_9R_N_1 | 25-25-25-25 |
| H4 | cl1_CD45_mSiglec_4GS_2_9R_C_1 | cl6_CD28_mCD86_4GS_2_9R_N_2 | cl4_CD28_mCD86_4GS_2_9R_N_1 | 25-25-25-25 |
| H5 | cl7_CD137_m41BBlg_4GS_2_9R_N_1 | cl12_IL2R_mIL2_4GS_2_9R_N_1 | cl4_CD28_mCD86_4GS_2_9R_N_1 | 25-25-25-25 |
| H6 | cl7_CD137_m41BBlg_4GS_2_9R_N_1 | cl2_CD28_mCD80_4GS_2_9R_N_1 | cl4_CD28_mCD86_4GS_2_9R_N_1 | 25-25-25-25 |
| H7 | cl12_IL2R_mIL2_4GS_2_9R_N_1 | cl6_CD28_mCD86_4GS_2_9R_N_2 | cl4_CD28_mCD86_4GS_2_9R_N_1 | 25-25-25-25 |
| H8 | cl11_CD3_CD3e_TFA_4GS_2_9R_N_1 | cl6_CD28_mCD86_4GS_2_9R_N_2 | cl9_CD3_mCD3Ab_4GS_2_9R_N_1 | 25-25-25-25 |
| H9 | cl2_CD28_mCD80_4GS_2_9R_N_1 | cl4_CD28_mCD86_4GS_2_9R_N_1 | cl14_ESELlg_mESEL_4GS_2_9R_N_1 | 25-25-25-25 |
| I10 | cl7_CD137_m41BBlg_4GS_2_9R_N_1 | cl11_CD3_CD3e_TFA_4GS_2_9R_N_1 | cl4_CD28_mCD86_4GS_2_9R_N_1 | 25-25-25-25 |
| I11 | cl7_CD137_m41BBlg_4GS_2_9R_N_1 | cl14_ESELlg_mESEL_4GS_2_9R_N_1 | cl9_CD3_mCD3Ab_4GS_2_9R_N_1 | 25-25-25-25 |
| I12 | cl12_IL2R_mIL2_4GS_2_9R_N_1 | cl2_CD28_mCD80_4GS_2_9R_N_1 | cl9_CD3_mCD3Ab_4GS_2_9R_N_1 | 25-25-25-25 |
| I13 | cl11_CD3_CD3e_TFA_4GS_2_9R_N_1 | cl4_CD28_mCD86_4GS_2_9R_N_1 | cl9_CD3_mCD3Ab_4GS_2_9R_N_1 | 25-25-25-25 |
| I14 | cl12_IL2R_mIL2_4GS_2_9R_N_1 | cl11_CD3_CD3e_TFA_4GS_2_9R_N_1 | cl6_CD28_mCD86_4GS_2_9R_N_2 | 25-25-25-25 |
| I15 | cl12_IL2R_mIL2_4GS_2_9R_N_1 | cl4_CD28_mCD86_4GS_2_9R_N_1 | cl14_ESELlg_mESEL_4GS_2_9R_N_1 | 25-25-25-25 |
| I16 | cl11_CD3_CD3e_TFA_4GS_2_9R_N_1 | cl14_ESELlg_mESEL_4GS_2_9R_N_1 | cl9_CD3_mCD3Ab_4GS_2_9R_N_1 | 25-25-25-25 |
| I17 | cl11_CD3_CD3e_TFA_4GS_2_9R_N_1 | cl6_CD28_mCD86_4GS_2_9R_N_2 | cl4_CD28_mCD86_4GS_2_9R_N_1 | 25-25-25-25 |
| I18 | cl6_CD28_mCD86_4GS_2_9R_N_2 | cl4_CD28_mCD86_4GS_2_9R_N_1 | cl14_ESELlg_mESEL_4GS_2_9R_N_1 | 25-25-25-25 |
| I19 | | | | |
| I21 | | | | |
| I3 | cl1_CD45_mSiglec_4GS_2_9R_C_1 | cl12_IL2R_mIL2_4GS_2_9R_N_1 | cl6_CD28_mCD86_4GS_2_9R_N_2 | 25-25-25-25 |
| I4 | cl1_CD45_mSiglec_4GS_2_9R_C_1 | cl6_CD28_mCD86_4GS_2_9R_N_2 | cl14_ESELlg_mESEL_4GS_2_9R_N_1 | 25-25-25-25 |
| I5 | cl7_CD137_m41BBlg_4GS_2_9R_N_1 | cl12_IL2R_mIL2_4GS_2_9R_N_1 | cl14_ESELlg_mESEL_4GS_2_9R_N_1 | 25-25-25-25 |
| I6 | cl7_CD137_m41BBlg_4GS_2_9R_N_1 | cl2_CD28_mCD80_4GS_2_9R_N_1 | cl14_ESELlg_mESEL_4GS_2_9R_N_1 | 25-25-25-25 |
| I7 | cl12_IL2R_mIL2_4GS_2_9R_N_1 | cl6_CD28_mCD86_4GS_2_9R_N_2 | cl14_ESELlg_mESEL_4GS_2_9R_N_1 | 25-25-25-25 |
| I8 | cl11_CD3_CD3e_TFA_4GS_2_9R_N_1 | cl2_CD28_mCD80_4GS_2_9R_N_1 | cl4_CD28_mCD86_4GS_2_9R_N_1 | 25-25-25-25 |
| I9 | cl2_CD28_mCD80_4GS_2_9R_N_1 | cl4_CD28_mCD86_4GS_2_9R_N_1 | cl9_CD3_mCD3Ab_4GS_2_9R_N_1 | 25-25-25-25 |
| J10 | cl7_CD137_m41BBlg_4GS_2_9R_N_1 | cl11_CD3_CD3e_TFA_4GS_2_9R_N_1 | cl14_ESELlg_mESEL_4GS_2_9R_N_1 | 25-25-25-25 |
| J11 | cl12_IL2R_mIL2_4GS_2_9R_N_1 | cl11_CD3_CD3e_TFA_4GS_2_9R_N_1 | cl6_CD28_mCD86_4GS_2_9R_N_2 | 25-25-25-25 |
| J12 | cl12_IL2R_mIL2_4GS_2_9R_N_1 | cl4_CD28_mCD86_4GS_2_9R_N_1 | cl14_ESELlg_mESEL_4GS_2_9R_N_1 | 25-25-25-25 |
| J13 | cl11_CD3_CD3e_TFA_4GS_2_9R_N_1 | cl14_ESELlg_mESEL_4GS_2_9R_N_1 | cl9_CD3_mCD3Ab_4GS_2_9R_N_1 | 25-25-25-25 |
| J14 | cl12_IL2R_mIL2_4GS_2_9R_N_1 | cl11_CD3_CD3e_TFA_4GS_2_9R_N_1 | cl2_CD28_mCD80_4GS_2_9R_N_1 | 25-25-25-25 |
| J15 | cl12_IL2R_mIL2_4GS_2_9R_N_1 | cl4_CD28_mCD86_4GS_2_9R_N_1 | cl9_CD3_mCD3Ab_4GS_2_9R_N_1 | 25-25-25-25 |
| J16 | cl6_CD28_mCD86_4GS_2_9R_N_2 | cl2_CD28_mCD80_4GS_2_9R_N_1 | cl4_CD28_mCD86_4GS_2_9R_N_1 | 25-25-25-25 |
| J17 | cl11_CD3_CD3e_TFA_4GS_2_9R_N_1 | cl6_CD28_mCD86_4GS_2_9R_N_2 | cl14_ESELlg_mESEL_4GS_2_9R_N_1 | 25-25-25-25 |
| J18 | cl6_CD28_mCD86_4GS_2_9R_N_2 | cl4_CD28_mCD86_4GS_2_9R_N_1 | cl9_CD3_mCD3Ab_4GS_2_9R_N_1 | 25-25-25-25 |
| J21 | | | | |
| J3 | | | | |
| J4 | cl1_CD45_mSiglec_4GS_2_9R_C_1 | cl12_IL2R_mIL2_4GS_2_9R_N_1 | cl2_CD28_mCD80_4GS_2_9R_N_1 | 25-25-25-25 |
| J5 | cl1_CD45_mSiglec_4GS_2_9R_C_1 | cl6_CD28_mCD86_4GS_2_9R_N_2 | cl9_CD3_mCD3Ab_4GS_2_9R_N_1 | 25-25-25-25 |
| J6 | cl7_CD137_m41BBlg_4GS_2_9R_N_1 | cl12_IL2R_mIL2_4GS_2_9R_N_1 | cl9_CD3_mCD3Ab_4GS_2_9R_N_1 | 25-25-25-25 |
| J7 | cl7_CD137_m41BBlg_4GS_2_9R_N_1 | cl2_CD28_mCD80_4GS_2_9R_N_1 | cl9_CD3_mCD3Ab_4GS_2_9R_N_1 | 25-25-25-25 |
| J8 | cl12_IL2R_mIL2_4GS_2_9R_N_1 | cl6_CD28_mCD86_4GS_2_9R_N_2 | cl9_CD3_mCD3Ab_4GS_2_9R_N_1 | 25-25-25-25 |
| J9 | cl11_CD3_CD3e_TFA_4GS_2_9R_N_1 | cl2_CD28_mCD80_4GS_2_9R_N_1 | cl14_ESELlg_mESEL_4GS_2_9R_N_1 | 25-25-25-25 |
| K10 | cl2_CD28_mCD80_4GS_2_9R_N_1 | cl14_ESELlg_mESEL_4GS_2_9R_N_1 | cl9_CD3_mCD3Ab_4GS_2_9R_N_1 | 25-25-25-25 |
| K11 | cl7_CD137_m41BBlg_4GS_2_9R_N_1 | cl11_CD3_CD3e_TFA_4GS_2_9R_N_1 | cl9_CD3_mCD3Ab_4GS_2_9R_N_1 | 25-25-25-25 |
| K12 | cl12_IL2R_mIL2_4GS_2_9R_N_1 | cl11_CD3_CD3e_TFA_4GS_2_9R_N_1 | cl2_CD28_mCD80_4GS_2_9R_N_1 | 25-25-25-25 |
| K13 | cl12_IL2R_mIL2_4GS_2_9R_N_1 | cl4_CD28_mCD86_4GS_2_9R_N_1 | cl9_CD3_mCD3Ab_4GS_2_9R_N_1 | 25-25-25-25 |
| K14 | cl6_CD28_mCD86_4GS_2_9R_N_2 | cl2_CD28_mCD80_4GS_2_9R_N_1 | cl4_CD28_mCD86_4GS_2_9R_N_1 | 25-25-25-25 |
| K15 | cl12_IL2R_mIL2_4GS_2_9R_N_1 | cl11_CD3_CD3e_TFA_4GS_2_9R_N_1 | cl4_CD28_mCD86_4GS_2_9R_N_1 | 25-25-25-25 |
| K16 | cl12_IL2R_mIL2_4GS_2_9R_N_1 | cl14_ESELlg_mESEL_4GS_2_9R_N_1 | cl9_CD3_mCD3Ab_4GS_2_9R_N_1 | 25-25-25-25 |
| K17 | cl6_CD28_mCD86_4GS_2_9R_N_2 | cl2_CD28_mCD80_4GS_2_9R_N_1 | cl14_ESELlg_mESEL_4GS_2_9R_N_1 | 25-25-25-25 |
| K18 | cl11_CD3_CD3e_TFA_4GS_2_9R_N_1 | cl6_CD28_mCD86_4GS_2_9R_N_2 | cl9_CD3_mCD3Ab_4GS_2_9R_N_1 | 25-25-25-25 |
| K21 | | | | |
| K3 | | | | |
| K4 | cl1_CD45_mSiglec_4GS_2_9R_C_1 | cl12_IL2R_mIL2_4GS_2_9R_N_1 | cl4_CD28_mCD86_4GS_2_9R_N_1 | 25-25-25-25 |
| K5 | cl1_CD45_mSiglec_4GS_2_9R_C_1 | cl2_CD28_mCD80_4GS_2_9R_N_1 | cl4_CD28_mCD86_4GS_2_9R_N_1 | 25-25-25-25 |
| K6 | cl7_CD137_m41BBlg_4GS_2_9R_N_1 | cl11_CD3_CD3e_TFA_4GS_2_9R_N_1 | cl6_CD28_mCD86_4GS_2_9R_N_2 | 25-25-25-25 |
| K7 | cl7_CD137_m41BBlg_4GS_2_9R_N_1 | cl4_CD28_mCD86_4GS_2_9R_N_1 | cl14_ESELlg_mESEL_4GS_2_9R_N_1 | 25-25-25-25 |
| K8 | cl12_IL2R_mIL2_4GS_2_9R_N_1 | cl2_CD28_mCD80_4GS_2_9R_N_1 | cl4_CD28_mCD86_4GS_2_9R_N_1 | 25-25-25-25 |
| K9 | cl11_CD3_CD3e_TFA_4GS_2_9R_N_1 | cl2_CD28_mCD80_4GS_2_9R_N_1 | cl9_CD3_mCD3Ab_4GS_2_9R_N_1 | 25-25-25-25 |
| L10 | cl4_CD28_mCD86_4GS_2_9R_N_1 | cl14_ESELlg_mESEL_4GS_2_9R_N_1 | cl9_CD3_mCD3Ab_4GS_2_9R_N_1 | 25-25-25-25 |
| L11 | cl7_CD137_m41BBlg_4GS_2_9R_N_1 | cl6_CD28_mCD86_4GS_2_9R_N_2 | cl2_CD28_mCD80_4GS_2_9R_N_1 | 25-25-25-25 |
| L12 | cl12_IL2R_mIL2_4GS_2_9R_N_1 | cl11_CD3_CD3e_TFA_4GS_2_9R_N_1 | cl4_CD28_mCD86_4GS_2_9R_N_1 | 25-25-25-25 |
| L13 | cl12_IL2R_mIL2_4GS_2_9R_N_1 | cl14_ESELlg_mESEL_4GS_2_9R_N_1 | cl9_CD3_mCD3Ab_4GS_2_9R_N_1 | 25-25-25-25 |
| L14 | cl6_CD28_mCD86_4GS_2_9R_N_2 | cl2_CD28_mCD80_4GS_2_9R_N_1 | cl14_ESELlg_mESEL_4GS_2_9R_N_1 | 25-25-25-25 |
| L15 | cl12_IL2R_mIL2_4GS_2_9R_N_1 | cl11_CD3_CD3e_TFA_4GS_2_9R_N_1 | cl14_ESELlg_mESEL_4GS_2_9R_N_1 | 25-25-25-25 |
| L16 | cl11_CD3_CD3e_TFA_4GS_2_9R_N_1 | cl6_CD28_mCD86_4GS_2_9R_N_2 | cl2_CD28_mCD80_4GS_2_9R_N_1 | 25-25-25-25 |
| L17 | cl6_CD28_mCD86_4GS_2_9R_N_2 | cl2_CD28_mCD80_4GS_2_9R_N_1 | cl9_CD3_mCD3Ab_4GS_2_9R_N_1 | 25-25-25-25 |
| L18 | cl11_CD3_CD3e_TFA_4GS_2_9R_N_1 | cl2_CD28_mCD80_4GS_2_9R_N_1 | cl4_CD28_mCD86_4GS_2_9R_N_1 | 25-25-25-25 |
| L21 | | | | |
| L3 | | | | |
| L4 | cl1_CD45_mSiglec_4GS_2_9R_C_1 | cl12_IL2R_mIL2_4GS_2_9R_N_1 | cl14_ESELlg_mESEL_4GS_2_9R_N_1 | 25-25-25-25 |
| L5 | cl1_CD45_mSiglec_4GS_2_9R_C_1 | cl2_CD28_mCD80_4GS_2_9R_N_1 | cl14_ESELlg_mESEL_4GS_2_9R_N_1 | 25-25-25-25 |
| L6 | cl7_CD137_m41BBlg_4GS_2_9R_N_1 | cl11_CD3_CD3e_TFA_4GS_2_9R_N_1 | cl2_CD28_mCD80_4GS_2_9R_N_1 | 25-25-25-25 |
| L7 | cl7_CD137_m41BBlg_4GS_2_9R_N_1 | cl4_CD28_mCD86_4GS_2_9R_N_1 | cl9_CD3_mCD3Ab_4GS_2_9R_N_1 | 25-25-25-25 |
| L8 | cl12_IL2R_mIL2_4GS_2_9R_N_1 | cl2_CD28_mCD80_4GS_2_9R_N_1 | cl14_ESELlg_mESEL_4GS_2_9R_N_1 | 25-25-25-25 |
| L9 | cl11_CD3_CD3e_TFA_4GS_2_9R_N_1 | cl4_CD28_mCD86_4GS_2_9R_N_1 | cl14_ESELlg_mESEL_4GS_2_9R_N_1 | 25-25-25-25 |
| M10 | cl7_CD137_m41BBlg_4GS_2_9R_N_1 | cl12_IL2R_mIL2_4GS_2_9R_N_1 | cl11_CD3_CD3e_TFA_4GS_2_9R_N_1 | 25-25-25-25 |
| M11 | cl7_CD137_m41BBlg_4GS_2_9R_N_1 | cl6_CD28_mCD86_4GS_2_9R_N_2 | cl4_CD28_mCD86_4GS_2_9R_N_1 | 25-25-25-25 |
| M12 | cl12_IL2R_mIL2_4GS_2_9R_N_1 | cl11_CD3_CD3e_TFA_4GS_2_9R_N_1 | cl14_ESELlg_mESEL_4GS_2_9R_N_1 | 25-25-25-25 |
| M13 | cl11_CD3_CD3e_TFA_4GS_2_9R_N_1 | cl6_CD28_mCD86_4GS_2_9R_N_2 | cl2_CD28_mCD80_4GS_2_9R_N_1 | 25-25-25-25 |
| M14 | cl6_CD28_mCD86_4GS_2_9R_N_2 | cl2_CD28_mCD80_4GS_2_9R_N_1 | cl9_CD3_mCD3Ab_4GS_2_9R_N_1 | 25-25-25-25 |
| M15 | cl12_IL2R_mIL2_4GS_2_9R_N_1 | cl11_CD3_CD3e_TFA_4GS_2_9R_N_1 | cl9_CD3_mCD3Ab_4GS_2_9R_N_1 | 25-25-25-25 |
| M16 | cl11_CD3_CD3e_TFA_4GS_2_9R_N_1 | cl6_CD28_mCD86_4GS_2_9R_N_2 | cl4_CD28_mCD86_4GS_2_9R_N_1 | 25-25-25-25 |
| M17 | cl6_CD28_mCD86_4GS_2_9R_N_2 | cl4_CD28_mCD86_4GS_2_9R_N_1 | cl14_ESELlg_mESEL_4GS_2_9R_N_1 | 25-25-25-25 |
| M18 | cl11_CD3_CD3e_TFA_4GS_2_9R_N_1 | cl2_CD28_mCD80_4GS_2_9R_N_1 | cl14_ESELlg_mESEL_4GS_2_9R_N_1 | 25-25-25-25 |
| M21 | | | | |
| M3 | cl1_CD45_mSiglec_4GS_2_9R_C_1 | cl7_CD137_m41BBlg_4GS_2_9R_N_1 | cl12_IL2R_mIL2_4GS_2_9R_N_1 | 25-25-25-25 |
| M4 | cl1_CD45_mSiglec_4GS_2_9R_C_1 | cl12_IL2R_mIL2_4GS_2_9R_N_1 | cl9_CD3_mCD3Ab_4GS_2_9R_N_1 | 25-25-25-25 |
| M5 | cl1_CD45_mSiglec_4GS_2_9R_C_1 | cl2_CD28_mCD80_4GS_2_9R_N_1 | cl9_CD3_mCD3Ab_4GS_2_9R_N_1 | 25-25-25-25 |
| M6 | cl7_CD137_m41BBlg_4GS_2_9R_N_1 | cl11_CD3_CD3e_TFA_4GS_2_9R_N_1 | cl4_CD28_mCD86_4GS_2_9R_N_1 | 25-25-25-25 |
| M7 | cl7_CD137_m41BBlg_4GS_2_9R_N_1 | cl14_ESELlg_mESEL_4GS_2_9R_N_1 | cl9_CD3_mCD3Ab_4GS_2_9R_N_1 | 25-25-25-25 |
| M8 | cl12_IL2R_mIL2_4GS_2_9R_N_1 | cl2_CD28_mCD80_4GS_2_9R_N_1 | cl9_CD3_mCD3Ab_4GS_2_9R_N_1 | 25-25-25-25 |
| M9 | cl11_CD3_CD3e_TFA_4GS_2_9R_N_1 | cl4_CD28_mCD86_4GS_2_9R_N_1 | cl9_CD3_mCD3Ab_4GS_2_9R_N_1 | 25-25-25-25 |
| N10 | cl7_CD137_m41BBlg_4GS_2_9R_N_1 | cl12_IL2R_mIL2_4GS_2_9R_N_1 | cl6_CD28_mCD86_4GS_2_9R_N_2 | 25-25-25-25 |
| N11 | cl7_CD137_m41BBlg_4GS_2_9R_N_1 | cl6_CD28_mCD86_4GS_2_9R_N_2 | cl14_ESELlg_mESEL_4GS_2_9R_N_1 | 25-25-25-25 |
| N12 | cl12_IL2R_mIL2_4GS_2_9R_N_1 | cl11_CD3_CD3e_TFA_4GS_2_9R_N_1 | cl9_CD3_mCD3Ab_4GS_2_9R_N_1 | 25-25-25-25 |
| N13 | cl11_CD3_CD3e_TFA_4GS_2_9R_N_1 | cl6_CD28_mCD86_4GS_2_9R_N_2 | cl4_CD28_mCD86_4GS_2_9R_N_1 | 25-25-25-25 |
| N14 | cl6_CD28_mCD86_4GS_2_9R_N_2 | cl4_CD28_mCD86_4GS_2_9R_N_1 | cl14_ESELlg_mESEL_4GS_2_9R_N_1 | 25-25-25-25 |
| N15 | cl12_IL2R_mIL2_4GS_2_9R_N_1 | cl6_CD28_mCD86_4GS_2_9R_N_2 | cl2_CD28_mCD80_4GS_2_9R_N_1 | 25-25-25-25 |
| N16 | cl11_CD3_CD3e_TFA_4GS_2_9R_N_1 | cl6_CD28_mCD86_4GS_2_9R_N_2 | cl14_ESELlg_mESEL_4GS_2_9R_N_1 | 25-25-25-25 |
| N17 | cl6_CD28_mCD86_4GS_2_9R_N_2 | cl4_CD28_mCD86_4GS_2_9R_N_1 | cl9_CD3_mCD3Ab_4GS_2_9R_N_1 | 25-25-25-25 |
| N18 | cl11_CD3_CD3e_TFA_4GS_2_9R_N_1 | cl2_CD28_mCD80_4GS_2_9R_N_1 | cl9_CD3_mCD3Ab_4GS_2_9R_N_1 | 25-25-25-25 |
| N3 | cl1_CD45_mSiglec_4GS_2_9R_C_1 | cl7_CD137_m41BBlg_4GS_2_9R_N_1 | cl11_CD3_CD3e_TFA_4GS_2_9R_N_1 | 25-25-25-25 |
| N4 | cl1_CD45_mSiglec_4GS_2_9R_C_1 | cl11_CD3_CD3e_TFA_4GS_2_9R_N_1 | cl6_CD28_mCD86_4GS_2_9R_N_2 | 25-25-25-25 |
| N5 | cl1_CD45_mSiglec_4GS_2_9R_C_1 | cl4_CD28_mCD86_4GS_2_9R_N_1 | cl14_ESELlg_mESEL_4GS_2_9R_N_1 | 25-25-25-25 |
| N6 | cl7_CD137_m41BBlg_4GS_2_9R_N_1 | cl11_CD3_CD3e_TFA_4GS_2_9R_N_1 | cl14_ESELlg_mESEL_4GS_2_9R_N_1 | 25-25-25-25 |
| N7 | cl12_IL2R_mIL2_4GS_2_9R_N_1 | cl11_CD3_CD3e_TFA_4GS_2_9R_N_1 | cl6_CD28_mCD86_4GS_2_9R_N_2 | 25-25-25-25 |
| N8 | cl12_IL2R_mIL2_4GS_2_9R_N_1 | cl4_CD28_mCD86_4GS_2_9R_N_1 | cl14_ESELlg_mESEL_4GS_2_9R_N_1 | 25-25-25-25 |
| N9 | cl11_CD3_CD3e_TFA_4GS_2_9R_N_1 | cl14_ESELlg_mESEL_4GS_2_9R_N_1 | cl9_CD3_mCD3Ab_4GS_2_9R_N_1 | 25-25-25-25 |

Table 18 depicts flow cytometry data for various nanoparticle variants. The first column depicts well location, while subsequent variables represent Cells_Count; % Live; % CD4+_LIVE; % CD8+_LIVE; % GFP_LIVE; Median SI GFP; % GFP_CD8; % GFP_CD4; % GFP(CD8-CD4); % Alexa594_GFP+; Ligand_1; Ligand_2; Ligand_3; Ligand_4; ratio (of ligands).

TABLE 19
depicts a comprehensive set of sgRNAs for Cas9 and Cpf1, TALENs, ssDNA, tetrisDNA and dsDNA
donors, recombinase-based site-specific gene insertion techniques, and the like. These
sequences were assessed for delivery efficiency via a multitude of means. Associated
primers for assessing cutting efficiency are included.
Sequences of nucleotides studied in the experiments showing supporting evidence for the
claims that follow

| | Nucleic | | |
|---|---|---|---|
| | Acid | | |
| Name | Type | Description | Sequence |
| LL001 | sgRNA | TRAC exon1 | TAATTTCTACTCTTGTAGATCATGTGCAAACGCCTTCAACAACA |
| | | Cpf1 guide | |
| LL002 | sgRNA | TRAC exon1 | TAATTTCTACTCTTGTAGATCATGTGCAAACGCCTTCAAC |
| | | Cpf1 guide | |
| LL003 | sgRNA | TRB1 exon1 | TAATTTCTACTCTTGTAGATGGTGTGGGAGATCTCTGCTTCTGA |
| | | Cpf1 guide- | |
| | | C1 and C2 | |
| LL004 | sgRNA | TRB promoter | TAATTTCTACTCTTGTAGATCAGATGGGCTGAAGTCTCCACTGT |
| | | Cpf1 guide | |
| LL005- | sgRNA | TRAC-519 | gcugguacacgccagggucaGTTTTAGAGCTAGAAATAGCAAGTTAAAATAAGGCTA |
| nuc6 | | Cas9 sgRNA | GTCCGTTATCAACTTGAAAAAGTGGCACCGAGTCGGTGCTTTTTTT |
| LL006- | sgRNA | TRAC-537 | uggauuuagagucucucagcGTTTTAGAGCTAGAAATAGCAAGTTAAAATAAGGCTA |
| nuc7 | | Cas9 sgRNA | GTCCGTTATCAACTTGAAAAAGTGGCACCGAGTCGGTGCTTTTTTT |
| LL007 | sgRNA | TRAC exon1 | GAGAATCAAAATCGGTGAATGTTTTAGAGCTAGAAATAGCAAGTTAAAATAA |
| | | Cas9 nickase | GGCTAGTCCGTTATCAACTTGAAAAAGTGGCACCGAGTCGGTGCTTTTTTT |
| | | guide(also | |
| | | use for WT | |
| | | spCas9) | |
| LL008 | sgRNA | TRAC exon1 | AACAAATGTGTCACAAAGTAGTTTTAGAGCTAGAAATAGCAAGTTAAAATAA |
| | | Cas9 nickase | GGCTAGTCCGTTATCAACTTGAAAAAGTGGCACCGAGTCGGTGCTTTTTTT |
| | | guide | |
| LL009 | sgRNA | TRA pro Cas9 | GAGCCACTGTAGTCTGCAGAGTTTTAGAGCTAGAAATAGCAAGTTAAAATAA |
| | | nickase | GGCTAGTCCGTTATCAACTTGAAAAAGTGGCACCGAGTCGGTGCTTTTTTT |
| | | guide(also | |
| | | use for WT | |
| | | spCas9) | |
| LL010 | sgRNA | TRA pro Cas9 | GGAACCGGGGATGCAGTGCCGTTTTAGAGCTAGAAATAGCAAGTTAAAATA |
| | | nickase guide | AGGCTAGTCCGTTATCAACTTGAAAAAGTGGCACCGAGTCGGTGCTTTTTTT |
| LL011 | sgRNA | TRBC exon1 | CAAACACAGCGACCTCGGGTGTTTTAGAGCTAGAAATAGCAAGTTAAAATAA |
| | | Cas9 nickase | GGCTAGTCCGTTATCAACTTGAAAAAGTGGCACCGAGTCGGTGCTTTTTTT |
| | | guide(also | |
| | | use for WT | |
| | | spCas9) | |
| LL012 | sgRNA | TRBC exon1 | AGAGATCTCCCACACCCAAAGTTTTAGAGCTAGAAATAGCAAGTTAAAATAA |
| | | Cas9 nickase | GGCTAGTCCGTTATCAACTTGAAAAAGTGGCACCGAGTCGGTGCTTTTTTT |
| | | guide | |
| LL013 | sgRNA | TRB pro Cas9 | CCCTGAGACAGGGGCTGCTTGTTTTAGAGCTAGAAATAGCAAGTTAAAATA |
| | | nickase guide | AGGCTAGTCCGTTATCAACTTGAAAAAGTGGCACCGAGTCGGTGCTTTTTTT |
| LL014 | sgRNA | TRB pro Cas9 | GGAAGCACACCCAGACGACAGTTTTAGAGCTAGAAATAGCAAGTTAAAATA |
| | | nickase | AGGCTAGTCCGTTATCAACTTGAAAAAGTGGCACCGAGTCGGTGCTTTTTTT |
| | | guide(also | |
| | | use for WT | |
| | | spCas9) | |
| LL015 | plasmid | TRAC exon1 | TGCCGTGTACCAGCTGAGA |
| | | left TALEN | |
| LL016 | plasmid | TRAC exon1 | TCGGTGAATAGGCAGACAG |
| | | right TALEN | |
| LL017 | plasmid | TRA promoter | TGGAGATAGGGACCTCAC |
| | | left TALEN | |
| LL018 | plasmid | TRA promoter | TGAGGCCAGGAACTGGAG |
| | | right TALEN | |
| LL019 | plasmid | TRBC exon1 | TGAACAAGGTGTTCCCAC |
| | | left TALEN | |
| LL020 | plasmid | TRBC exon1 | TCTGCTTCTGATGGCTCA |
| | | right TALEN | |
| LL021 | plasmid | TRB promoter | TGTCTCAGGGCCAGGGAA |
| | | left TALEN | |
| LL022 | plasmid | TRB promoter | TCCCTGCTCTGTGTCCTT |
| | | right TALEN | |
| LL023 | sgRNA | TRA promoter | cttctctatgtttccatgaagatg |
| | | Cpf1 guide | |
| LL024- | sgRNA | HBB Cas9 | gtaacggcagacttctcctcGTTTTAGAGCTAGAAATAGCAAGTTAAAATAAGGCTAGT |
| nuc4 | | guide | CCGTTATCAACTTGAAAAAGTGGCACCGAGTCGGTGCTTTTTTT |
| LL025 | ssDNA | TRAC exon1 | CAACggctccggcgagggcaggggaagtctactaacatgcggggacgtggaggaaaatcccggccca |
| | | cpf1 sfGFP | agcaaaggagaagaacttttcactggagttgtcccaattcttgttgaattagatggtgatgttaatg |
| | | Tetris donor | ggcacaaattttctgtccgtggagagggtgaaggtgatgctacaaacggaaaactcacccttaaatt |
| | | sense | tatttgcactactggaaaactacctgttccgtggccaacacttgtcactactctgacctatggtgtt |
| | | | caatgcttttcccgttatccggatcacatgaaacggcatgactttttcaagagtgccatgcccgaag |
| | | | gttatgtacaggaacgcactatatctttcaaagatgacgggacctacaagacgcgtgctgaagtcaa |
| | | | gtttgaaggtgatacccttgttaatcgtatcgagttaaagggtattgattttaaagaagatggaaac |
| | | | attcttggacacaaactcgagtacaactttaactcacacaatgtatacatcacggcagacaaacaaa |
| | | | agaatggaatcaaagctaacttcaaaattcgccacaacgttgaagatggttccgttcaactagcaga |
| | | | ccattatcaacaaaatactccaattggcgatggccctgtccttttaccagacaaccattacctgtcg |
| | | | acacaatctgtcctttcgaaagatcccaacgaaaagcgtgaccacatggtccttcttgagtttgtaa |
| | | | ctgctgctgggattacacatggcatggatgagctctacaaaTAATAG |
| LL026 | ssDNA | TRAC exon1 | gttgCTATTAtttgtagagctcatccatgccatgtgtaatcccagcagcagttacaaactcaagaag |
| | | cpf1 sfGFP | gaccatgtggtcacgcttttcgttgggatctttcgaaaggacagattgtgtcgacaggtaatggttg |
| | | Tetris donor | tctggtaaaaggacagggccatcgccaattggagtattttgttgataatggtctgctagttgaacgg |
| | | antisense | aaccatcttcaacgttgtggcgaattttgaagttagctttgattccattcttttgtttgtctgccgt |
| | | | gatgtatacattgtgtgagttaaagttgtactcgagtttgtgtccaagaatgtttccatcttcttta |
| | | | aaatcaataccctttaactcgatacgattaacaagggtatcaccttcaaacttgacttcagcacgcg |
| | | | tcttgtaggtcccgtcatctttgaaagatatagtgcgttcctgtacataaccttcgggcatggcact |
| | | | cttgaaaaagtcatgccgtttcatgtgatccggataacgggaaaagcattgaacaccataggtcaga |
| | | | gtagtgacaagtgttggccacggaacaggtagttttccagtagtgcaaataaatttaagggtgagtt |
| | | | ttccgtttgtagcatcaccttcaccctctccacggacagaaaatttgtgcccattaacatcaccatc |
| | | | taattcaacaagaattgggacaactccagtgaaaagttcttctcctttgcttgggccgggattttcc |
| | | | tccacgtccccgcatgttagtagacttcccctgccctcgccggagcc |
| LL027 | ssDNA, | TRAC exon1 | GGAGACCACTCCAGATTCCAAGATGTACAGTTTGCTTTGCTGGGCCTTTTTC |
| | dsDNA | Cas9 HDR | CCATGCCTGCCTTTACTCTGCCAGAGTTATATTGCTGGGGTTTTGAAGAAGA |
| | | sfGFP donor | TCCTATTAAATAAAAGAATAAGCAGTATTATTAAGTAGCCCTGCATTTCAGGT |
| | | (LL07 and | TTCCTTGAGTGGCAGGCCAGGCCTGGCCGTGAACGTTCACTGAAATCATG |
| | | LL08 guides) | GCCTCTTGGCCAAGATTGATAGCTTGTGCCTGTCCCTGAGTCCCAGTCCAT |
| | | | CACGAGCAGCTGGTTTCTAAGATGCTATTTCCCGTATAAAGCATGAGACCG |
| | | | TGACTTGCCAGCCCCACAGAGCCCCGCCCTTGTCCATCACTGGCATCTGG |
| | | | ACTCCAGCCTGGGTTGGGGCAAAGAGGGAAATGAGATCATGTCCTAACCC |
| | | | TGATCCTCTTGTCCCACAGATATCCAGAACCCTGACCCTGCCGTGTACCAG |
| | | | CTGAGAGACTCTAAATCCAGTGACAAGTCTGTCTGCCTAGGCTCCGGCGAG |
| | | | GGCAGGGGAAGTCTACTAACATGCGGGGACGTGGAGGAAAATCCCGGCC |
| | | | CAAGCAAAGGAGAAGAACTTTTCACTGGAGTTGTCCCAATTCTTGTTGAATT |
| | | | AGATGGTGATGTTAATGGGCACAAATTTTCTGTCCGTGGAGAGGGTGAAGG |
| | | | TGATGCTACAAACGGAAAACTCACCCTTAAATTTATTTGCACTACTGGAAAA |
| | | | CTACCTGTTCCGTGGCCAACACTTGTCACTACTCTGACCTATGGTGTTCAAT |
| | | | GCTTTTCCCGTTATCCGGATCACATGAAACGGCATGACTTTTTCAAGAGTGC |
| | | | CATGCCCGAAGGTTATGTACAGGAACGCACTATATCTTTCAAAGATGACGG |
| | | | GACCTACAAGACGCGTGCTGAAGTCAAGTTTGAAGGTGATACCCTTGTTAA |
| | | | TCGTATCGAGTTAAAGGGTATTGATTTTAAAGAAGATGGAAACATTCTTGGA |
| | | | CACAAACTCGAGTACAACTTTAACTCACACAATGTATACATCACGGCAGACA |
| | | | AACAAAAGAATGGAATCAAAGCTAACTTCAAAATTCGCCACAACGTTGAAGA |
| | | | TGGTTCCGTTCAACTAGCAGACCATTATCAACAAAATACTCCAATTGGCGAT |
| | | | GGCCCTGTCCTTTTACCAGACAACCATTACCTGTCGACACAATCTGTCCTTT |
| | | | CGAAAGATCCCAACGAAAAGCGTGACCACATGGTCCTTCTTGAGTTTGTAA |
| | | | CTGCTGCTGGGATTACACATGGCATGGATGAGCTCTACAAATAATAGACCG |
| | | | ATTTTGATTCTCAAACAAATGTGTCACAAAGTAAGGATTCTGATGTGTATATC |
| | | | ACAGACAAAACTGTGCTAGACATGAGGTCTATGGACTTCAAGAGCAACAGT |
| | | | GCTGTGGCCTGGAGCAACAAATCTGACTTTGCATGTGCAAACGCCTTCAAC |
| | | | AACAGCATTATTCCAGAAGACACCTTCTTCCCCAGCCCAGGTAAGGGCAGC |
| | | | TTTGGTGCCTTCGCAGGCTGTTTCCTTGCTTCAGGAATGGCCAGGTTCTGC |
| | | | CCAGAGCTCTGGTCAATGATGTCTAAAACTCCTCTGATTGGTGGTCTCGGC |
| | | | CTTATCCATTGCCACCAAAACCCTCTTTTTACTAAGAAACAGTGAGCCTTGT |
| | | | TCTGGCAGTCCAGAGAATGACACGGGAAAAAAGCAGATGAAGAGAAGGTG |
| | | | GCAGGAGAGGGCACGTGGCCCAGCCTCAGTCTCTCCAACTGAGTTCCTGC |
| | | | CTGCCTGCCTTTGCTCAGACTGTTTGCCCCTTACTG |
| LL028 | dsDNA | TRAC exon1 | GAACGTTCACTGAAATCATGGCCTCTTGGCCAAGATTGATAGCTTGTGCCT |
| | | Cpf1 HDR | GTCCCTGAGTCCCAGTCCATCACGAGCAGCTGGTTTCTAAGATGCTATTTC |
| | | sfGFP donor | CCGTATAAAGCATGAGACCGTGACTTGCCAGCCCCACAGAGCCCCGCCCT |
| | | | TGTCCATCACTGGCATCTGGACTCCAGCCTGGGTTGGGGCAAAGAGGGAA |
| | | | ATGAGATCATGTCCTAACCCTGATCCTCTTGTCCCACAGATATCCAGAACCC |
| | | | TGACCCTGCCGTGTACCAGCTGAGAGACTCTAAATCCAGTGACAAGTCTGT |
| | | | CTGCCTATTCACCGATTTTGATTCTCAAACAAATGTGTCACAAAGTAAGGAT |
| | | | TCTGATGTGTATATCACAGACAAAACTGTGCTAGACATGAGGTCTATGGACT |
| | | | TCAAGAGCAACAGTGCTGTGGCCTGGAGCAACAAATCTGACATAGCATGTG |
| | | | CAAACGCCTTCGGCTCCGGCGAGGGCAGGGGAAGTCTACTAACATGCGG |
| | | | GGACGTGGAGGAAAATCCCGGCCCAAGCAAAGGAGAAGAACTTTTCACTG |
| | | | GAGTTGTCCCAATTCTTGTTGAATTAGATGGTGATGTTAATGGGCACAAATT |
| | | | TTCTGTCCGTGGAGAGGGTGAAGGTGATGCTACAAACGGAAAACTCACCCT |
| | | | TAAATTTATTTGCACTACTGGAAAACTACCTGTTCCGTGGCCAACACTTGTC |
| | | | ACTACTCTGACCTATGGTGTTCAATGCTTTTCCCGTTATCCGGATCACATGA |
| | | | AACGGCATGACTTTTTCAAGAGTGCCATGCCCGAAGGTTATGTACAGGAAC |
| | | | GCACTATATCTTTCAAAGATGACGGGACCTACAAGACGCGTGCTGAAGTCA |
| | | | AGTTTGAAGGTGATACCCTTGTTAATCGTATCGAGTTAAAGGGTATTGATTT |
| | | | TAAAGAAGATGGAAACATTCTTGGACACAAACTCGAGTACAACTTTAACTCA |
| | | | CACAATGTATACATCACGGCAGACAAACAAAAGAATGGAATCAAAGCTAACT |
| | | | TCAAAATTCGCCACAACGTTGAAGATGGTTCCGTTCAACTAGCAGACCATTA |
| | | | TCAACAAAATACTCCAATTGGCGATGGCCCTGTCCTTTTACCAGACAACCAT |
| | | | TACCTGTCGACACAATCTGTCCTTTCGAAAGATCCCAACGAAAAGCGTGAC |
| | | | CACATGGTCCTTCTTGAGTTTGTAACTGCTGCTGGGATTACACATGGCATG |
| | | | GATGAGCTCTACAAATAATAGAACAGCATTATTCCAGAAGACACCTTCTTCC |
| | | | CCAGCCCAGGTAAGGGCAGCTTTGGTGCCTTCGCAGGCTGTTTCCTTGCTT |
| | | | CAGGAATGGCCAGGTTCTGCCCAGAGCTCTGGTCAATGATGTCTAAAACTC |
| | | | CTCTGATTGGTGGTCTCGGCCTTATCCATTGCCACCAAAACCCTCTTTTTAC |
| | | | TAAGAAACAGTGAGCCTTGTTCTGGCAGTCCAGAGAATGACACGGGAAAAA |
| | | | AGCAGATGAAGAGAAGGTGGCAGGAGAGGGCACGTGGCCCAGCCTCAGT |
| | | | CTCTCCAACTGAGTTCCTGCCTGCCTGCCTTTGCTCAGACTGTTTGCCCCT |
| | | | TACTGCTCTTCTAGGCCTCATTCTAAGCCCCTTCTCCAAGTTGCCTCTCCTT |
| | | | ATTTCTCCCTGTCTGCCAAAAAATCTTTCCCAGCTCACTAAGTCAGTCTCAC |
| | | | GCAGTCACTCATTAACCCACCAATCACTGATTGTGC |
| LL029 | ssDNA, | Trac exon1 | ggagaccactccagattccaagatgtacagtttgctttgctgggcctttttcccatgcctgccttta |
| | dsDNA | Cas9 mckase | ctctgccagagttatattgctggggttttgaagaagatcctattaaataaaagaataagcagtatta |
| | | HDR sfGFP | ttaagtagccctgcatttcaggtttccttgagtggcaggccaggcctggccgtgaacgttcactgaa |
| | | donor | atcatggcctcttggccaagattgatagcttgtgcctgtccctgagtcccagtccatcacgagcagc |
| | | | tggtttctaagatgctatttcccgtataaagcatgagaccgtgacttgccagccccacagagccccg |
| | | | cccttgtccatcactggcatctggactccagcctgggttggggcaaagagggaaatgagatcatgtc |
| | | | ctaaccctgatcctcttgtcccacagatatccagaaccctgaccctgccgtgtaccagctgagagac |
| | | | tctaaatccagtgacaagtctgtctgcctaggctccggcgagggcaggggaagtctactaacatgcg |
| | | | gggacgtggaggaaaatcccggcccaagcaaaggagaagaacttttcactggagttgtcccaattct |
| | | | tgttgaattagatggtgatgttaatgggcacaaattttctgtccgtggagagggtgaaggtgatgct |
| | | | acaaacggaaaactcacccttaaatttatttgcactactggaaaactacctgttccgtggccaacac |
| | | | ttgtcactactctgacctatggtgttcaatgcttttcccgttatccggatcacatgaaacggcatga |
| | | | ctttttcaagagtgccatgcccgaaggttatgtacaggaacgcactatatctttcaaagatgacggg |
| | | | acctacaagacgcgtgctgaagtcaagtttgaaggtgatacccttgttaatcgtatcgagttaaagg |
| | | | gtattgattttaaagaagatggaaacattcttggacacaaactcgagtacaactttaactcacacaa |
| | | | tgtatacatcacggcagacaaacaaaagaatggaatcaaagctaacttcaaaattcgccacaacgtt |
| | | | gaagatggttccgttcaactagcagaccattatcaacaaaatactccaattggcgatggccctgtcc |
| | | | ttttaccagacaaccattacctgtcgacacaatctgtcctttcgaaagatcccaacgaaaagcgtga |
| | | | ccacatggtccttcttgagtttgtaactgctgctgggattacacatggcatggatgagctctacaaa |
| | | | TAATAGgtaaggattctgatgtgtatatcacagacaaaactgtgctagacatgaggtctatggactt |
| | | | caagagcaacagtgctgtggcctggagcaacaaatctgactttgcatgtgcaaacgccttcaacaac |
| | | | agcattattccagaagacaccttcttccccagcccaggtaagggcagctttggtgccttcgcaggct |
| | | | gtttccttgcttcaggaatggccaggttctgcccagagctctggtcaatgatgtctaaaactcctct |
| | | | gattggtggtctcggccttatccattgccaccaaaaccctctttttactaagaaacagtgagccttg |
| | | | ttctggcagtccagagaatgacacgggaaaaaagcagatgaagagaaggtggcaggagagggcacgt |
| | | | ggcccagcctcagtctctccaactgagttcctgcctgcctgcctttgctcagactgtttgcccctta |
| | | | ctgctcttctaggcctcattctaagccccttctcca |
| LL030 | ssDNA | TRAC exon1 | attcggctccggcgagggcaggggaagtctactaacatgcggggacgtggaggaaaatcccggccca |
| | | double cpf1 | agcaaaggagaagaacttttcactggagttgtcccaattcttgttgaattagatggtgatgttaatg |
| | | Tetris donor | ggcacaaattttctgtccgtggagagggtgaaggtgatgctacaaacggaaaactcacccttaaatt |
| | | sense-anneal | tatttgcactactggaaaactacctgttccgtggccaacacttgtcactactctgacctatggtgtt |
| | | with LL031 | caatgcttttcccgttatccggatcacatgaaacggcatgactttttcaagagtgccatgcccgaag |
| | | | gttatgtacaggaacgcactatatctttcaaagatgacgggacctacaagacgcgtgctgaagtcaa |
| | | | gtttgaaggtgatacccttgttaatcgtatcgagttaaagggtattgattttaaagaagatggaaac |
| | | | attcttggacacaaactcgagtacaactttaactcacacaatgtatacatcacggcagacaaacaaa |
| | | | agaatggaatcaaagctaacttcaaaattcgccacaacgttgaagatggttccgttcaactagcaga |
| | | | ccattatcaacaaaatactccaattggcgatggccctgtccttttaccagacaaccattacctgtcg |
| | | | acacaatctgtcctttcgaaagatcccaacgaaaagcgtgaccacatggtccttcttgagtttgtaa |
| | | | ctgctgctgggattacacatggcatggatgagctctacaaaTAATAG |
| LL031 | ssDNA | TRAC exon1 | gttgCTATTAtttgtagagctcatccatgccatgtgtaatcccagcagcagttacaaactcaagaag |
| | | double cpf1 | gaccatgtggtcacgcttttcgttgggatctttcgaaaggacagattgtgtcgacaggtaatggttg |
| | | Tetris donor | tctggtaaaaggacagggccatcgccaattggagtattttgttgataatggtctgctagttgaacgg |
| | | antisense- | aaccatcttcaacgttgtggcgaattttgaagttagctttgattccattcttttgtttgtctgccgt |
| | | anneal with | gatgtatacattgtgtgagttaaagttgtactcgagtttgtgtccaagaatgtttccatcttcttta |
| | | LL030 | aaatcaataccctttaactcgatacgattaacaagggtatcaccttcaaacttgacttcagcacgcg |
| | | | tcttgtaggtcccgtcatctttgaaagatatagtgcgttcctgtacataaccttcgggcatggcact |
| | | | cttgaaaaagtcatgccgtttcatgtgatccggataacgggaaaagcattgaacaccataggtcaga |
| | | | gtagtgacaagtgttggccacggaacaggtagttttccagtagtgcaaataaatttaagggtgagtt |
| | | | ttccgtttgtagcatcaccttcaccctctccacggacagaaaatttgtgcccattaacatcaccatc |
| | | | taattcaacaagaattgggacaactccagtgaaaagttcttctcctttgcttgggccgggattttcc |
| | | | tccacgtccccgcatgttagtagacttcccctgccctcgccggagcc |
| LL032 | sgRNA | TRAC exon1 | tttgagaatcaaaatcggtg |
| | | cpf1 guide2, | |
| | | use with | |
| | | LL001, double | |
| | | cut to delete | |
| | | cpf1 PAM for | |
| | | tetris donor- | |
| | | anneal LL030 | |
| | | and 031- | |
| | | overlap with | |
| | | LL007, can | |
| | | also use LL027 | |
| | | for HDR donor | |
| LL033 | | TRBC C1C2 | aaatatatacatcttgatttaaaaaaggaaaattataattagaaaaagtcaatttagttattgtaat |
| | | HDR donor, | tataccactaatgagagtttcctacctcgagtttcaggattacatagccatgcaccaagcaaggctt |
| | | can use with | tgaaaaataaagatacacagataaattatttggatagatgatcagacaagcctcagtaaaaacagcc |
| | | Cas9-nickase- | aagacaatcaggatataatgtgaccataggaagctggggagacagtaggcaatgtgcatccatggga |
| | | Cpf1-TALEN, | cagcatagaaaggaggggcaaagtggagagagagcaacagacactgggatggtgaccccaaaacaat |
| | | suppose | gagggcctagaatgacatagttgtgcttcattacggcccattcccagggctctctctcacacacaca |
| | | deletion | gagcccctaccagaaccagacagctctcagagcaaccctggctccaacccctcttccctttccagag |
| | | between C1C2 | gacctgaacaaggtgttcccaggctccggcgagggcaggggaagtctactaacatgcggggacgtgg |
| | | | aggaaaatcccggcccaagcaaaggagaagaacttttcactggagttgtcccaattcttgttgaatt |
| | | | agatggtgatgttaatgggcacaaattttctgtccgtggagagggtgaaggtgatgctacaaacgga |
| | | | aaactcacccttaaatttatttgcactactggaaaactacctgttccgtggccaacacttgtcacta |
| | | | ctctgacctatggtgttcaatgcttttcccgttatccggatcacatgaaacggcatgactttttcaa |
| | | | gagtgccatgcccgaaggttatgtacaggaacgcactatatctttcaaagatgacgggacctacaag |
| | | | acgcgtgctgaagtcaagtttgaaggtgatacccttgttaatcgtatcgagttaaagggtattgatt |
| | | | ttaaagaagatggaaacattcttggacacaaactcgagtacaactttaactcacacaatgtatacat |
| | | | cacggcagacaaacaaaagaatggaatcaaagctaacttcaaaattcgccacaacgttgaagatggt |
| | | | tccgttcaactagcagaccattatcaacaaaatactccaattggcgatggccctgtccttttaccag |
| | | | acaaccattacctgtcgacacaatctgtcctttcgaaagatcccaacgaaaagcgtgaccacatggt |
| | | | ccttcttgagtttgtaactgctgctgggattacacatggcatggatgagctctacaaaTAATAGgcc |
| | | | acactggtatgcctggccacaggcttctaccccgaccacgtggagctgagctggtgggtgaatggga |
| | | | aggaggtgcacagtggggtcagcacagacccgcagcccctcaaggagcagcccgccctcaatgactc |
| | | | cagatactgcctgagcagccgcctgagggtctcggccaccttctggcagaacccccgcaaccacttc |
| | | | cgctgtcaagtccagttctacgggctctcggagaatgacgagtggacccaggatagggccaaacccg |
| | | | tcacccagatcgtcagcgccgaggcctggggtagagcaggtgagtggggcctggggagatgcctgga |
| | | | ggagattaggtgagaccagctaccagggaaaatggaaagatccaggtagcggacaagactagatcca |
| | | | gaagaaagccagagtggacaaggtgggatgatcaaggttcacagggtcagcaaagcacggtgtgcac |
| | | | ttccc |
| LL034 | ssDNA | TRAC c1c2 | CAGAggctccggcgagggcaggggaagtctactaacatgcggggacgtggaggaaaatcccggccca |
| | | double cpf1 | agcaaaggagaagaacttttcactggagttgtcccaattcttgttgaattagatggtgatgttaatg |
| | | Tetris donor | ggcacaaattttctgtccgtggagagggtgaaggtgatgctacaaacggaaaactcacccttaaatt |
| | | sense | tatttgcactactggaaaactacctgttccgtggccaacacttgtcactactctgacctatggtgtt |
| | | | caatgcttttcccgttatccggatcacatgaaacggcatgactttttcaagagtgccatgcccgaag |
| | | | gttatgtacaggaacgcactatatctttcaaagatgacgggacctacaagacgcgtgctgaagtcaa |
| | | | gtttgaaggtgatacccttgttaatcgtatcgagttaaagggtattgattttaaagaagatggaaac |
| | | | attcttggacacaaactcgagtacaactttaactcacacaatgtatacatcacggcagacaaacaaa |
| | | | agaatggaatcaaagctaacttcaaaattcgccacaacgttgaagatggttccgttcaactagcaga |
| | | | ccattatcaacaaaatactccaattggcgatggccctgtccttttaccagacaaccattacctgtcg |
| | | | acacaatctgtcctttcgaaagatcccaacgaaaagcgtgaccacatggtccttcttgagtttgtaa |
| | | | ctgctgctgggattacacatggcatggatgagctctacaaaTAATAG |
| LL035 | ssDNA | TRAC c1c2 | TCTGCTATTAtttgtagagctcatccatgccatgtgtaatcccagcagcagttacaaactcaagaag |
| | | double cpf1 | gaccatgtggtcacgcttttcgttgggatctttcgaaaggacagattgtgtcgacaggtaatggttg |
| | | Tetris donor | tctggtaaaaggacagggccatcgccaattggagtattttgttgataatggtctgctagttgaacgg |
| | | antisense | aaccatcttcaacgttgtggcgaattttgaagttagctttgattccattcttttgtttgtctgccgt |
| | | | gatgtatacattgtgtgagttaaagttgtactcgagtttgtgtccaagaatgtttccatcttcttta |
| | | | aaatcaataccctttaactcgatacgattaacaagggtatcaccttcaaacttgacttcagcacgcg |
| | | | tcttgtaggtcccgtcatctttgaaagatatagtgcgttcctgtacataaccttcgggcatggcact |
| | | | cttgaaaaagtcatgccgtttcatgtgatccggataacgggaaaagcattgaacaccataggtcaga |
| | | | gtagtgacaagtgttggccacggaacaggtagttttccagtagtgcaaataaatttaagggtgagtt |
| | | | ttccgtttgtagcatcaccttcaccctctccacggacagaaaatttgtgcccattaacatcaccatc |
| | | | taattcaacaagaattgggacaactccagtgaaaagttcttctcctttgcttgggccgggattttcc |
| | | | tccacgtccccgcatgttagtagacttcccctgccctcgccggagcc |
| LL036 | sgRNA | trac pro | ACCATACTAACAGTTTTCTTTCTC |
| | | deletion cpf1 | |
| | | guide | |
| LL037 | sgRNA | trac pro | actgcatctctaattgatcc |
| | | deletion cas9 | |
| | | guide (use | |
| | | with LL007) | |
| LL038 | ssDNA | TRA deletion | TGATCTGCCTGCCTTGGCCTCCCAAAGTGGTGGGATTACAGGTGTGAGCC |
| | | cpf1 donor | ACTGCTCCCAGCTCTTTTTTCCTGTTATACCTCTTTTCTTTCCTTTAGTTTTTT |
| | | | AAAAAATTACATAATCAAACATGTCTATTTTAACATTAACCAATAGAGGGATG |
| | | | TACCAAAAAAAATTAACTCAACTCACTGCAACCCACTGCAACCCCTGACATA |
| | | | ACCAATGTTAGTAGTTTATTGAGTATATCCTCACACTTTTAAAAATGTATGCA |
| | | | TATGTACATAAGTTTATGATAAAAATATCATTCAATACTCATCACTCTGCAAC |
| | | | TTACTTTTGAATATATTAAAGATTATTTCTATATTAGCTGTTGTAAGCACACTT |
| | | | AAATGGTAGGTAAATTTCCTTGTCTTTCTAGCTTCCAAAATATATATGACACA |
| | | | CAAACAAACAATATTTAGTATATGCACACACACACTGCATCTCTAATTGATCC |
| | | | TGGATTTCATTTTGTTGAGTCACCCAAGTGTGGTCTAATATAAATCCTGTGTT |
| | | | CCTGAGGTCATGCAGATTGAGAGAGGAAGTGATGTCACTGTGGGAACTTCC |
| | | | GTGTAAGGACGGGGCGTCCCTCCTCCTCTGCTCCTGCTCACAGTGATCCT |
| | | | GATCTGGTAAGAGCTCCCATCCTGCCCTGACCCTGCCATGAGCAAGGGCG |
| | | | AGGAGCTGTTCACCGGGGTGGTGCCCATCCTGGTCGAGCTGGACGGCGA |
| | | | CGTAAACGGCCACAAGTTCAGCGTGCGTGGCGAGGGCGAGGGCGATGCC |
| | | | ACCAACGGCAAGCTGACCCTGAAGTTCATCTGCACCACCGGCAAGCTGCC |
| | | | CGTGCCCTGGCCCACCCTCGTGACCACCCTGACCTACGGCGTGCAGTGCT |
| | | | TCAGCCGCTACCCCGACCACATGAAGCGTCACGACTTCTTCAAGTCCGCCA |
| | | | TGCCCGAAGGCTACGTCCAGGAGCGCACCATCTCGTTCAAGGACGACGGC |
| | | | ACATACAAGACCCGCGCCGAGGTGAAGTTCGAGGGCGACACCCTGGTGAA |
| | | | CCGCATCGAGCTGAAGGGCATCGACTTCAAGGAGGACGGCAACATCCTGG |
| | | | GGCACAAGCTGGAGTACAACTTTAACAGCCACAACGTCTATATCACAGCCG |
| | | | ACAAGCAGAAGAACGGCATCAAGGCAAACTTCAAGATCCGCCACAACGTTG |
| | | | AGGACGGCAGCGTGCAGCTCGCCGACCACTACCAGCAGAACACCCCCATC |
| | | | GGCGACGGCCCCGTGCTGCTGCCCGACAACCACTACCTGAGCACCCAGTC |
| | | | CGTTCTGAGCAAAGACCCCAACGAGAAGCGCGATCACATGGTCCTGCTGG |
| | | | AGTTCGTGACCGCCGCCGGGATCACTCACGGCATGGACGAGCTGTACAAG |
| | | | TAATAGATTCCAGAAGACACCTTCTTCCCCAGCCCAGGTAAGGGCAGCTTT |
| | | | GGTGCCTTCGCAGGCTGTTTCCTTGCTTCAGGAATGGCCAGGTTCTGCCCA |
| | | | GAGCTCTGGTCAATGATGTCTAAAACTCCTCTGATTGGTGGTCTCGGCCTT |
| | | | ATCCATTGCCACCAAAACCCTCTTTTTACTAAGAAACAGTGAGCCTTGTTCT |
| | | | GGCAGTCCAGAGAATGACACGGGAAAAAAGCAGATGAAGAGAAGGTGGCA |
| | | | GGAGAGGGCACGTGGCCCAGCCTCAGTCTCTCCAACTGAGTTCCTGCCTG |
| | | | CCTGCCTTTGCTCAGACTGTTTGCCCCTTACTGCTCTTCTAGGCCTCATTCT |
| | | | AAGCCCCTTCTCCAAGTTGCCTCTCCTTATTTCTCCCTGTCTGCCAAAAAAT |
| | | | CTTTCCCAGCTCACTAAGTCAGTCTCACGCAGTCACTCATTAACCCACCAAT |
| | | | CACTGATTGTGCCGGCACATGAATGCACCAGGTGTTGAAGTGGAG |
| LL039 | ssDNA | TRA promoter | GTATATGCACACACACACTGCATCTCTAATTGATCCTGGATTTCATTTTGTTG |
| | | Cas9 donor | TATCATGAGAAAGAAAACTGTTAGTATGGTCAAATTGATTAGTTTTGACTTTG |
| | | | CCTTATGTTCCCATTTGTTTTCTCTGTTCTTTACATGTTCGATGTTCACCATA |
| | | | ATCACTTGGATTAAAATGTGTGGATTAGTTTTTGGAGATAGGGACCTCACCA |
| | | | TGTTGCTTAGGCTGGTCTCCAGTTCCTGGCCTCAAGGGATTCTTCTACCTC |
| | | | AGCGTCTTGAGTAGCTGGGATTACAGGCATAAGCCACTGTGCCCAGCTTAA |
| | | | AACCTGTGGATTTATCAGTAGAAAATGTTCATGTAAAGATACTCCTGTAAGA |
| | | | GAAACCATAGCTGCTCCAGTGGAAGGAAGCTTAAACTCATCCCTTCAAGAA |
| | | | AGAAGCTCCTCCCTTTGTATTTCTACTGGGTTTTGCATCCGGACTGATCTTC |
| | | | CTTCCCTCACCCACATGAAGTGTCTAACTTCTGCAGACTACAGTGGCTCAG |
| | | | GAACCGGGGATGCAGTGCCAGGCTCATGGTATCCTGCAGCAGATGAGCAA |
| | | | AGGAGAAGAACTTTTCACTGGAGTTGTCCCAATTCTTGTTGAATTAGATGGT |
| | | | GATGTTAATGGGCACAAATTTTCTGTCCGTGGAGAGGGTGAAGGTGATGCT |
| | | | ACAAACGGAAAACTCACCCTTAAATTTATTTGCACTACTGGAAAACTACCTG |
| | | | TTCCGTGGCCAACACTTGTCACTACTCTGACCTATGGTGTTCAATGCTTTTC |
| | | | CCGTTATCCGGATCACATGAAACGGCATGACTTTTTCAAGAGTGCCATGCC |
| | | | CGAAGGTTATGTACAGGAACGCACTATATCTTTCAAAGATGACGGGACCTA |
| | | | CAAGACGCGTGCTGAAGTCAAGTTTGAAGGTGATACCCTTGTTAATCGTAT |
| | | | CGAGTTAAAGGGTATTGATTTTAAAGAAGATGGAAACATTCTTGGACACAAA |
| | | | CTCGAGTACAACTTTAACTCACACAATGTATACATCACGGCAGACAAACAAA |
| | | | AGAATGGAATCAAAGCTAACTTCAAAATTCGCCACAACGTTGAAGATGGTTC |
| | | | CGTTCAACTAGCAGACCATTATCAACAAAATACTCCAATTGGCGATGGCCCT |
| | | | GTCCTTTTACCAGACAACCATTACCTGTCGACACAATCTGTCCTTTCGAAAG |
| | | | ATCCCAACGAAAAGCGTGACCACATGGTCCTTCTTGAGTTTGTAACTGCTG |
| | | | CTGGGATTACACATGGCATGGATGAGCTCTACAAATAATAGGCAGACTACA |
| | | | GTGGCTCAGGAACCGGGGATGCAGTGCCAGGCTCATGGTATCCTGCAGCAG |
| LL040 | plasmid | Tra promoter | gtatatgcacacacacactgcatctctaattgatcctggatttcattttgttgtatcatgagaaaga |
| | | Cas9 donor | aaactgttagtatggtcaaattgattagttttgactttgccttatgttcccatttgttttctctgtt |
| | | | ctttacatgttcgatgttcaccataatcacttggattaaaatgtgtggattagtttttggagatagg |
| | | | gacctcaccatgttgcttaggctggtctccagttcctggcctcaagggattcttctacctcagcgtc |
| | | | ttgagtagctgggattacaggcataagccactgtgcccagcttaaaacctgtggatttatcagtaga |
| | | | aaatgttcatgtaaagatactcctgtaagagaaaccatagctgctccagtggaaggaagcttaaact |
| | | | catcccttcaagaaagaagctcctccctttgtatttctactgggttttgcatccggactgatcttcc |
| | | | ttccctcacccacatgaagtgtctaActtctgcagactacagtggctcaggaaccggggatgcagtg |
| | | | ccaggctcatggtatcctgcagcagATGagcaaaggagaagaacttttcactggagttgtcccaatt |
| | | | cttgttgaattagatggtgatgttaatgggcacaaattttctgtccgtggagagggtgaaggtgatg |
| | | | ctacaaacggaaaactcacccttaaatttatttgcactactggaaaactacctgttccgtggccaac |
| | | | acttgtcactactctgacctatggtgttcaatgcttttcccgttatccggatcacatgaaacggcat |
| | | | gactttttcaagagtgccatgcccgaaggttatgtacaggaacgcactatatctttcaaagatgacg |
| | | | ggacctacaagacgcgtgctgaagtcaagtttgaaggtgatacccttgttaatcgtatcgagttaaa |
| | | | gggtattgattttaaagaagatggaaacattcttggacacaaactcgagtacaactttaactcacac |
| | | | aatgtatacatcacggcagacaaacaaaagaatggaatcaaagctaacttcaaaattcgccacaacg |
| | | | ttgaagatggttccgttcaactagcagaccattatcaacaaaatactccaattggcgatggccctgt |
| | | | ccttttaccagacaaccattacctgtcgacacaatctgtcctttcgaaagatcccaacgaaaagcgt |
| | | | gaccacatggtccttcttgagtttgtaactgctgctgggattacacatggcatggatgagctctaca |
| | | | aaTAATAGgcagactacagtggctcaggaaccggggatgcagtgccaggctcatggtatcctgcagc |
| | | | ag |
| LL041 | plasmid | Tra pro cpf1 | atccggactgatcttccttccctcacccacatgaagtgtctaccttctgcagactacagtggctcag |
| | | tetris sense | gaaccggggatgcagtgccaggctcatggtatcctgcagcagATGagcaaaggagaagaacttttca |
| | | | ctggagttgtcccaattcttgttgaattagatggtgatgttaatgggcacaaattttctgtccgtgg |
| | | | agagggtgaaggtgatgctacaaacggaaaactcacccttaaatttatttgcactactggaaaacta |
| | | | cctgttccgtggccaacacttgtcactactctgacctatggtgttcaatgcttttcccgttatccgg |
| | | | atcacatgaaacggcatgactttttcaagagtgccatgcccgaaggttatgtacaggaacgcactat |
| | | | atctttcaaagatgacgggacctacaagacgcgtgctgaagtcaagtttgaaggtgatacccttgtt |
| | | | aatcgtatcgagttaaagggtattgattttaaagaagatggaaacattcttggacacaaactcgagt |
| | | | acaactttaactcacacaatgtatacatcacggcagacaaacaaaagaatggaatcaaagctaactt |
| | | | caaaattcgccacaacgttgaagatggttccgttcaactagcagaccattatcaacaaaatactcca |
| | | | attggcgatggccctgtccttttaccagacaaccattacctgtcgacacaatctgtcctttcgaaag |
| | | | atcccaacgaaaagcgtgaccacatggtccttcttgagtttgtaactgctgctgggattacacatgg |
| | | | catggatgagctctacaaaTAATAG |
| LL042 | plasmid | Tra pro cpf1 | GGATCTATTAtttgtagagctcatccatgccatgtgtaatcccagcagcagttacaaactcaagaag |
| | | tetris | gaccatgtggtcacgcttttcgttgggatctttcgaaaggacagattgtgtcgacaggtaatggttg |
| | | antisense | tctggtaaaaggacagggccatcgccaattggagtattttgttgataatggtctgctagttgaacgg |
| | | | aaccatcttcaacgttgtggcgaattttgaagttagctttgattccattcttttgtttgtctgccgt |
| | | | gatgtatacattgtgtgagttaaagttgtactcgagtttgtgtccaagaatgtttccatcttcttta |
| | | | aaatcaataccctttaactcgatacgattaacaagggtatcaccttcaaacttgacttcagcacgcg |
| | | | tcttgtaggtcccgtcatctttgaaagatatagtgcgttcctgtacataaccttcgggcatggcact |
| | | | cttgaaaaagtcatgccgtttcatgtgatccggataacgggaaaagcattgaacaccataggtcaga |
| | | | gtagtgacaagtgttggccacggaacaggtagttttccagtagtgcaaataaatttaagggtgagtt |
| | | | ttccgtttgtagcatcaccttcaccctctccacggacagaaaatttgtgcccattaacatcaccatc |
| | | | taattcaacaagaattgggacaactccagtgaaaagttcttctcctttgctCATctgctgcaggata |
| | | | ccatgagcctggcactgcatccccggttcctgagccactgtagtctgcagaaggtagacacttcatg |
| | | | tgggtgagggaaggaagatcagtcc |
| LL043 | plasmid | CMV TagBFP-N | |
| LL044 | plasmid | CMV TagGFP2-N | |
| LL045 | plasmid | CMV TagRFP-N | |
| LL046 | plasmid | Trbc1&2 | tcttgatttaaaaaaggaaaattataattagaaaaagtcaatttagttattgtaattataccactaa |
| | | exon1 HDR | tgagagtttcctacctcgagtttcaggattacatagccatgcaccaagcaaggctttgaaaaataaa |
| | | sfGFP donor | gatacacagataaattatttggatagatgatcagacaagcctcagtaaaaacagccaagacaatcag |
| | | | gatataatgtgaccataggaagctggggagacagtaggcaatgtgcatccatgggacagcatagaaa |
| | | | ggaggggcaaagtggagagagagcaacagacactgggatggtgaccccaaaacaatgagggcctaga |
| | | | atgacatagttgtgcttcattacggcccattcccagggctctctctcacacacacagagcccctacc |
| | | | agaaccagacagctctcagagcaaccctggctccaacccctcttccctttccagaggacctgaacaa |
| | | | ggtgttcccagctccggcgagggcaggggaagtctactaacatgcggggacgtggaggaaaatcccg |
| | | | gcccaagcaaaggagaagaacttttcactggagttgtcccaattcttgttgaattagatggtgatgt |
| | | | taatgggcacaaattttctgtccgtggagagggtgaaggtgatgctacaaacggaaaactcaccctt |
| | | | aaatttatttgcactactggaaaactacctgttccgtggccaacacttgtcactactctgacctatg |
| | | | gtgttcaatgcttttcccgttatccggatcacatgaaacggcatgactttttcaagagtgccatgcc |
| | | | cgaaggttatgtacaggaacgcactatatctttcaaagatgacgggacctacaagacgcgtgctgaa |
| | | | gtcaagtttgaaggtgatacccttgttaatcgtatcgagttaaagggtattgattttaaagaagatg |
| | | | gaaacattcttggacacaaactcgagtacaactttaactcacacaatgtatacatcacggcagacaa |
| | | | acaaaagaatggaatcaaagctaacttcaaaattcgccacaacgttgaagatggttccgttcaacta |
| | | | gcagaccattatcaacaaaatactccaattggcgatggccctgtccttttaccagacaaccattacc |
| | | | tgtcgacacaatctgtcctttcgaaagatcccaacgaaaagcgtgaccacatggtccttcttgagtt |
| | | | tgtaactgctgctgggattacacatggcatggatgagctctacaaaTAATAGgccacactggtatgc |
| | | | ctggccacaggcttctaccccgaccacgtggagctgagctggtgggtgaatgggaaggaggtgcaca |
| | | | gtggggtcagcacagacccgcagcccctcaaggagcagcccgccctcaatgactccagatactgcct |
| | | | gagcagccgcctgagggtctcggccaccttctggcagaacccccgcaaccacttccgctgtcaagtc |
| | | | cagttctacgggctctcggagaatgacgagtggacccaggatagggccaaacccgtcacccagatcg |
| | | | tcagcgccgaggcctggggtagagcaggtgagtggggcctggggagatgcctggaggagattaggtg |
| | | | agaccagctaccagggaaaatggaaagatccaggtagcggacaagactagatccagaagaaagccag |
| | | | agtggacaaggtgggatgatcaaggttcacagggtcagcaaagcacggtgtgcacttccc |
| LL047 | plasmid | TRDC exon1 | gtttggctccagggtaatcgaggtaatcaccactgtttaacccccacaaagttgtgaataatcatct |
| | | cpf1 2A-sfGFP | cacctaataagttgattatatttgcaggaagtcagcctcataccaaaccatccgtttttgtcatgaa |
| | | donor | aaatggaacaaatgtcgcttgtctggtgaaggaattctaccccaaggatataagaataaatctcgtg |
| | | | tcatccaagaagataacagagtttgatcctgctattgtcatctctcccagtgggaagtacaatgctg |
| | | | tcaagcttggtaaatatgaagattcaaattcagtgggctccggcgagggcaggggaagtctactaac |
| | | | atgcggggacgtggaggaaaatcccggcccaagcaaaggagaagaacttttcactggagttgtccca |
| | | | attcttgttgaattagatggtgatgttaatgggcacaaattttctgtccgtggagagggtgaaggtg |
| | | | atgctacaaacggaaaactcacccttaaatttatttgcactactggaaaactacctgttccgtggcc |
| | | | aacacttgtcactactctgacctatggtgttcaatgcttttcccgttatccggatcacatgaaacgg |
| | | | catgactttttcaagagtgccatgcccgaaggttatgtacaggaacgcactatatctttcaaagatg |
| | | | acgggacctacaagacgcgtgctgaagtcaagtttgaaggtgatacccttgttaatcgtatcgagtt |
| | | | aaagggtattgattttaaagaagatggaaacattcttggacacaaactcgagtacaactttaactca |
| | | | cacaatgtatacatcacggcagacaaacaaaagaatggaatcaaagctaacttcaaaattcgccaca |
| | | | acgttgaagatggttccgttcaactagcagaccattatcaacaaaatactccaattggcgatggccc |
| | | | tgtccttttaccagacaaccattacctgtcgacacaatctgtcctttcgaaagatcccaacgaaaag |
| | | | cgtgaccacatggtccttcttgagtttgtaactgctgctgggattacacatggcatggatgagctct |
| | | | acaaaTAATAGgttcagttcaacacgacaataCaGctgtgcactccactgactttgaagtgaagaca |
| | | | gattctacaggtaggccatttctagcttcaaggagctggagattatggggaacaagaattgggtgaa |
| | | | agggaagttagagatgtaactgtggacaaatcattctcagtatagcatcatgctggaaataagactt |
| | | | aggcccaactatagcctgccattggcaggggagggaaatgcttgtcatccctaagatggaatctaaa |
| | | | ataaagcccatcttatttcttcctcatctctcctctttacctacca |
| LL049 | dsDNA | TRBC cas9 | CCCACGAGACAAATATATACATCTTGATTTAAAAAAGGAAAATTATAATTAGA |
| | (PCR), | dsRed2 donor | AAAAGTCAATTTAGTTATTGTAATTATACCACTAATGAGAGTTTCCTACCTCG |
| | plasmid | | AGTTTCAGGATTACATAGCCATGCACCAAGCAAGGCTTTGAAAAATAAAGAT |
| | | | ACACAGATAAATTATTTGGATAGATGATCAGACAAGCCTCAGTAAAAACAGC |
| | | | CAAGACAATCAGGATATAATGTGACCATAGGAAGCTGGGGAGACAGTAGG |
| | | | CAATGTGCATCCATGGGACAGCATAGAAAGGAGGGGCAAAGTGGAGAGAG |
| | | | AGCAACAGACACTGGGATGGTGACCCCAAAACAATGAGGGCCTAGAATGA |
| | | | CATAGTTGTGCTTCATTACGGCCCATTCCCAGGGCTCTCTCTCACACACAC |
| | | | AGAGCCCCTACCAGAACCAGACAGCTCTCAGAGCAACCCTGGCTCCAACC |
| | | | CCTCTTCCCTTTCCAGAGGACCTGAACAAGGTGTTCCCAGGCTCCGGCGA |
| | | | GGGCAGGGGAAGTCTACTAACATGCGGGGACGTGGAGGAAAATCCCGGC |
| | | | CCAGCCTCCTCCGAGAACGTCATCACCGAGTTCATGCGCTTCAAGGTGCG |
| | | | CATGGAGGGCACCGTGAACGGCCACGAGTTCGAGATCGAGGGCGAGGGC |
| | | | GAGGGCCGCCCCTACGAGGGCCACAACACCGTGAAGCTGAAGGTGACCA |
| | | | AGGGCGGCCCCCTGCCCTTCGCCTGGGACATCCTGTCCCCCCAGTTCCAG |
| | | | TACGGCTCCAAGGTGTACGTGAAGCACCCCGCCGACATCCCCGACTACAA |
| | | | GAAGCTGTCCTTCCCCGAGGGCTTCAAGTGGGAGCGCGTGATGAACTTCG |
| | | | AGGACGGCGGCGTGGCGACCGTGACCCAGGACTCCTCCCTGCAGGACGG |
| | | | CTGCTTCATCTACAAGGTGAAGTTCATCGGCGTGAACTTCCCCTCCGACGG |
| | | | CCCCGTGATGCAGAAGAAGACCATGGGCTGGGAGGCCTCCACCGAGCGC |
| | | | CTGTACCCCCGCGACGGCGTGCTGAAGGGCGAGACCCACAAGGCCCTGA |
| | | | AGCTGAAGGACGGCGGCCACTACCTGGTGGAGTTCAAGTCCATCTACATG |
| | | | GCCAAGAAGCCCGTGCAGCTGCCCGGCTACTACTACGTGGACGCCAAGCT |
| | | | GGACATCACCTCCCACAACGAGGACTACACCATCGTGGAGCAGTACGAGC |
| | | | GCACCGAGGGCCGCCACCACCTGTTCCTGTAGCGAGGTCGCTGTGTTTGA |
| | | | GCCATCAGAAGCAGAGATCTCCCACACCCAAAAGGCCACACTGGTATGCCT |
| | | | GGCCACAGGCTTCTACCCCGACCACGTGGAGCTGAGCTGGTGGGTGAATG |
| | | | GGAAGGAGGTGCACAGTGGGGTCAGCACAGACCCGCAGCCCCTCAAGGA |
| | | | GCAGCCCGCCCTCAATGACTCCAGATACTGCCTGAGCAGCCGCCTGAGGG |
| | | | TCTCGGCCACCTTCTGGCAGAACCCCCGCAACCACTTCCGCTGTCAAGTCC |
| | | | AGTTCTACGGGCTCTCGGAGAATGACGAGTGGACCCAGGATAGGGCCAAA |
| | | | CCCGTCACCCAGATCGTCAGCGCCGAGGCCTGGGGTAGAGCAGGTGAGT |
| | | | GGGGCCTGGGGAGATGCCTGGAGGAGATTAGGTGAGACCAGCTACCAGG |
| | | | GAAAATGGAAAGATCCAGGTAGCGGACAAGACTAGATCCAGAAGAAAGCC |
| | | | AGAGTGGACAAGGTGGGATGATCAAGGTTCACA |
| LL050 | plasmid | TRBC cas9n | agtaaaaacagccaagacaatcaggatataatgtgaccataggaagctggggagacagtaggcaatg |
| | | RFP donor | tgcatccatgggacagcatagaaaggaggggcaaagtggagagagagcaacagacactgggatggtg |
| | | | accccaaaacaatgagggcctagaatgacatagttgtgcttcattacggcccattcccagggctctc |
| | | | tctcacacacacagagcccctaccagaaccagacagctctcagagcaaccctggctccaacccctct |
| | | | tccctttccagaggacctgaacaaggtgttcccaggctccggcgagggcaggggaagtctactaaca |
| | | | tgcggggacgtggaggaaaatcccggcccagcctcctccgagaacgtcatcaccgagttcatgcgct |
| | | | tcaaggtgcgcatggagggcaccgtgaacggccacgagttcgagatcgagggcgagggcgagggccg |
| | | | cccctacgagggccacaacaccgtgaagctgaaggtgaccaagggcggccccctgcccttcgcctgg |
| | | | gacatcctgtccccccagttccagtacggctccaaggtgtacgtgaagcaccccgccgacatccccg |
| | | | actacaagaagctgtccttccccgagggcttcaagtgggagcgcgtgatgaacttcgaggacggcgg |
| | | | cgtggcgaccgtgacccaggactcctccctgcaggacggctgcttcatctacaaggtgaagttcatc |
| | | | ggcgtgaacttcccctccgacggccccgtgatgcagaagaagaccatgggctgggaggcctccaccg |
| | | | agcgcctgtacccccgcgacggcgtgctgaagggcgagacccacaaggccctgaagctgaaggacgg |
| | | | cggccactacctggtggagttcaagtccatctacatggccaagaagcccgtgcagctgcccggctac |
| | | | tactacgtggacgccaagctggacatcacctcccacaacgaggactacaccatcgtggagcagtacg |
| | | | agcgcaccgagggccgccaccacctgttcctgtagaaaaggccacactggtatgcctggccacaggc |
| | | | ttctaccccgaccacgtggagctgagctggtgggtgaatgggaaggaggtgcacagtggggtcagca |
| | | | cagacccgcagcccctcaaggagcagcccgccctcaatgactccagatactgcctgagcagccgcct |
| | | | gagggtctcggccaccttctggcagaacccccgcaaccacttccgctgtcaagtccagttctacggg |
| | | | ctctcggagaatgacgagtggacccaggatagggccaaacccgtcacccagatcgtcagcgccgagg |
| | | | cc |
| LL051 | plasmid | TRBC cpf1 | CCATGCACCAAGCAAGGCTTTGAAAAATAAAGATACACAGATAAATTATTTG |
| | | dsRed2 donor | GATAGATGATCAGACAAGCCTCAGTAAAAACAGCCAAGACAATCAGGATAT |
| | | | AATGTGACCATAGGAAGCTGGGGAGACAGTAGGCAATGTGCATCCATGGG |
| | | | ACAGCATAGAAAGGAGGGGCAAAGTGGAGAGAGAGCAACAGACACTGGGA |
| | | | TGGTGACCCCAAAACAATGAGGGCCTAGAATGACATAGTTGTGCTTCATTA |
| | | | CGGCCCATTCCCAGGGCTCTCTCTCACACACACAGAGCCCCTACCAGAAC |
| | | | CAGACAGCTCTCAGAGCAACCCTGGCTCCAACCCCTCTTCCCTTTCCAGAG |
| | | | GACCTGAACAAGGTGTTCCCACCCGAGGTCGCTGTGTTTGAGCCAGGCTC |
| | | | CGGCGAGGGCAGGGGAAGTCTACTAACATGCGGGGACGTGGAGGAAAAT |
| | | | CCCGGCCCAGCCTCCTCCGAGAACGTCATCACCGAGTTCATGCGCTTCAA |
| | | | GGTGCGCATGGAGGGCACCGTGAACGGCCACGAGTTCGAGATCGAGGGC |
| | | | GAGGGCGAGGGCCGCCCCTACGAGGGCCACAACACCGTGAAGCTGAAGG |
| | | | TGACCAAGGGCGGCCCCCTGCCCTTCGCCTGGGACATCCTGTCCCCCCAG |
| | | | TTCCAGTACGGCTCCAAGGTGTACGTGAAGCACCCCGCCGACATCCCCGA |
| | | | CTACAAGAAGCTGTCCTTCCCCGAGGGCTTCAAGTGGGAGCGCGTGATGA |
| | | | ACTTCGAGGACGGCGGCGTGGCGACCGTGACCCAGGACTCCTCCCTGCA |
| | | | GGACGGCTGCTTCATCTACAAGGTGAAGTTCATCGGCGTGAACTTCCCCTC |
| | | | CGACGGCCCCGTGATGCAGAAGAAGACCATGGGCTGGGAGGCCTCCACC |
| | | | GAGCGCCTGTACCCCCGCGACGGCGTGCTGAAGGGCGAGACCCACAAGG |
| | | | CCCTGAAGCTGAAGGACGGCGGCCACTACCTGGTGGAGTTCAAGTCCATC |
| | | | TACATGGCCAAGAAGCCCGTGCAGCTGCCCGGCTACTACTACGTGGACGC |
| | | | CAAGCTGGACATCACCTCCCACAACGAGGACTACACCATCGTGGAGCAGT |
| | | | ACGAGCGCACCGAGGGCCGCCACCACCTGTTCCTGTAGAGCAGAGATCTC |
| | | | CCACACCCAAAAGGCCACACTGGTATGCCTGGCCACAGGCTTCTACCCCG |
| | | | ACCACGTGGAGCTGAGCTGGTGGGTGAATGGGAAGGAGGTGCACAGTGG |
| | | | GGTCAGCACAGACCCGCAGCCCCTCAAGGAGCAGCCCGCCCTCAATGACT |
| | | | CCAGATACTGCCTGAGCAGCCGCCTGAGGGTCTCGGCCACCTTCTGGCAG |
| | | | AACCCCCGCAACCACTTCCGCTGTCAAGTCCAGTTCTACGGGCTCTCGGA |
| | | | GAATGACGAGTGGACCCAGGATAGGGCCAAACCCGTCACCCAGATCGTCA |
| | | | GCGCCGAGGCCTGGGGTAGAGCAGGTGAGTGGGGCCTGGGGAGATGCCT |
| | | | GGAGGAGATTAGGTGAGACCAGCTACCAGGGAAAATGGA |
| LL052 | plasmid | TRB pro cas9 | CTTAAAATGATGCACAGCTGGCTCCAGGGAAGGGCTCCACTGAGCTAGGT |
| | | dsRed2 donor | GAGGTGTCCTCCTGGAATTCACTGAGATGAGGGAGGGGAGCTGGAGTGTG |
| | | | CTCATCCTGGGTCCAAGACAGGCATCGGGAAGGCATCTGCCCAAAGGGAA |
| | | | GGGGTCTGTGTGTTAGGGAGGAGGGGAGCCATAAGTAGAAAGAGGAAGG |
| | | | GGAGACCCATTCATTCGTTGTGGGAAGGGCAGGCAGCTGCTAAGAAAAAA |
| | | | GCAACTGTCTAAAGAACCCGCCCTGCACACCTGGCCCTGAGAAGCTAGTCT |
| | | | AAACCCACCTCTTGAGGTGCCAGTGCCAAGCTTGGAAAGGAAAGAGGAAG |
| | | | TGTGAGCTGTAGACACTAATAGTGACACCAACAGGAGCAGAGACTTCCCAA |
| | | | GCAGCCCCTGTCTCAGGGCCAGGGAAGCACACCCAGACGACAAGGACAC |
| | | | AGAGCAGGGAGACACAGGGTCCCCCTGCCTGTGCCCCGGGTGACCCTGC |
| | | | CGGCTCCGGCGAGGGCAGGGGAAGTCTACTAACATGCGGGGACGTGGAG |
| | | | GAAAATCCCGGCCCAGCCTCCTCCGAGAACGTCATCACCGAGTTCATGCG |
| | | | CTTCAAGGTGCGCATGGAGGGCACCGTGAACGGCCACGAGTTCGAGATCG |
| | | | AGGGCGAGGGCGAGGGCCGCCCCTACGAGGGCCACAACACCGTGAAGCT |
| | | | GAAGGTGACCAAGGGCGGCCCCCTGCCCTTCGCCTGGGACATCCTGTCCC |
| | | | CCCAGTTCCAGTACGGCTCCAAGGTGTACGTGAAGCACCCCGCCGACATC |
| | | | CCCGACTACAAGAAGCTGTCCTTCCCCGAGGGCTTCAAGTGGGAGCGCGT |
| | | | GATGAACTTCGAGGACGGCGGCGTGGCGACCGTGACCCAGGACTCCTCC |
| | | | CTGCAGGACGGCTGCTTCATCTACAAGGTGAAGTTCATCGGCGTGAACTTC |
| | | | CCCTCCGACGGCCCCGTGATGCAGAAGAAGACCATGGGCTGGGAGGCCT |
| | | | CCACCGAGCGCCTGTACCCCCGCGACGGCGTGCTGAAGGGCGAGACCCA |
| | | | CAAGGCCCTGAAGCTGAAGGACGGCGGCCACTACCTGGTGGAGTTCAAGT |
| | | | CCATCTACATGGCCAAGAAGCCCGTGCAGCTGCCCGGCTACTACTACGTG |
| | | | GACGCCAAGCTGGACATCACCTCCCACAACGAGGACTACACCATCGTGGA |
| | | | GCAGTACGAGCGCACCGAGGGCCGCCACCACCTGTTCCTGTAGACAAGGA |
| | | | CACAGAGCAGGGAGACACAGGGTCCCCCTGCCTGTGCCCCGGGTGACCC |
| | | | TGCCATGGGCTGAAGTCTCCACTGTGGTGTGGTCCATTGTCTCAGGTGAGT |
| | | | CCTGGGCACAGGTGGGACATTTCTGTCCTTAAATTTTTTGCTTTTTTCATGG |
| | | | AACTGCTTCAGAAGATTCTGTCCTAGGCTTAGTCTGAATTTGGCTTCTTATTT |
| | | | TCATAGGCTCCATGGATACTGGAATTACCCAGACACCAAAATACCTGGTCA |
| | | | CAGCAATGGGGAGTAAAAGGACAATGAAACGTGAGCATCTGGGACATGATT |
| | | | CTATGTATTGGTACAGACAGAAAGCTAAGAAATCCCTGGAGTTCATGTTTTA |
| | | | CTACAACTGTAAGGAATTCATTGAAAACAAGACTGTGCCAAATCACTTCACA |
| | | | CCTGAATGCCCT |
| LL053 | sgRNA | TRB pro cpf1 | CAAGCTTGGCACTGGCACCTCAAG |
| | | guide | |
| LL054 | plasmid | TRB pro cpf1 | CTTAAAATGATGCACAGCTGGCTCCAGGGAAGGGCTCCACTGAGCTAGGT |
| | | dsRed2 donor | GAGGTGTCCTCCTGGAATTCACTGAGATGAGGGAGGGGAGCTGGAGTGTG |
| | | | CTCATCCTGGGTCCAAGACAGGCATCGGGAAGGCATCTGCCCAAAGGGAA |
| | | | GGGGTCTGTGTGTTAGGGAGGAGGGGAGCCATAAGTAGAAAGAGGAAGG |
| | | | GGAGACCCATTCATTCGTTGTGGGAAGGGCAGGCAGCTGCTAAGAAAAAA |
| | | | GCAACTGTCTAAAGAACCCGCCCTGCACACCTGGCCCTGAGAAGCTAGTCT |
| | | | AAACCCACCTCTTGAGGTGCCAGTGCCAAGCTTGGAAAGGAAAGAGGAAG |
| | | | TGTGAGCTGTAGACACTAATAGTGACACCAACAGGAGCAGAGACTTCCCAA |
| | | | GCAGCCCCTGTCTCAGGGCCAGGGAAGCACACCCAGACGACAAGGACAC |
| | | | AGAGCAGGGAGACACAGGGTCCCCCTGCCTGTGCCCCGGGTGACCCTGC |
| | | | CGGCTCCGGCGAGGGCAGGGGAAGTCTACTAACATGCGGGGACGTGGAG |
| | | | GAAAATCCCGGCCCAGCCTCCTCCGAGAACGTCATCACCGAGTTCATGCG |
| | | | CTTCAAGGTGCGCATGGAGGGCACCGTGAACGGCCACGAGTTCGAGATCG |
| | | | AGGGCGAGGGCGAGGGCCGCCCCTACGAGGGCCACAACACCGTGAAGCT |
| | | | GAAGGTGACCAAGGGCGGCCCCCTGCCCTTCGCCTGGGACATCCTGTCCC |
| | | | CCCAGTTCCAGTACGGCTCCAAGGTGTACGTGAAGCACCCCGCCGACATC |
| | | | CCCGACTACAAGAAGCTGTCCTTCCCCGAGGGCTTCAAGTGGGAGCGCGT |
| | | | GATGAACTTCGAGGACGGCGGCGTGGCGACCGTGACCCAGGACTCCTCC |
| | | | CTGCAGGACGGCTGCTTCATCTACAAGGTGAAGTTCATCGGCGTGAACTTC |
| | | | CCCTCCGACGGCCCCGTGATGCAGAAGAAGACCATGGGCTGGGAGGCCT |
| | | | CCACCGAGCGCCTGTACCCCCGCGACGGCGTGCTGAAGGGCGAGACCCA |
| | | | CAAGGCCCTGAAGCTGAAGGACGGCGGCCACTACCTGGTGGAGTTCAAGT |
| | | | CCATCTACATGGCCAAGAAGCCCGTGCAGCTGCCCGGCTACTACTACGTG |
| | | | GACGCCAAGCTGGACATCACCTCCCACAACGAGGACTACACCATCGTGGA |
| | | | GCAGTACGAGCGCACCGAGGGCCGCCACCACCTGTTCCTGTAGGGTGCC |
| | | | AGTGCCAAGCTTGGGACGGAAAGAGGAAGTGTGAGCTGTAGACACTAATA |
| | | | GTGACACCAACAGGAGCAGAGACTTCCCAAGCAGCCCCTGTCTCAGGGCC |
| | | | AGGGAAGCACACCCAGACGACAAGGACACAGAGCAGGGAGACACAGGGT |
| | | | CCCCCTGCCTGTGCCCCGGGTGACCCTGCCATGGGCTGAAGTCTCCACTG |
| | | | TGGTGTGGTCCATTGTCTCAGGTGAGTCCTGGGCACAGGTGGGACATTTCT |
| | | | GTCCTTAAATTTTTTGCTTTTTTCATGGAACTGCTTCAGAAGA |
| LL055 | plasmid | TRB pro cpf1 | ttgaggtgccagtgccaagcttggaaaggaaagaggaagtgtgagctgtagacactaatagtgacac |
| | | RFP tefris | caacaggagcagagacttcccaagcagcccctgtctcagggccagggaagcacacccagacgacaag |
| | | donor sense | gacacagagcagggagacacagggtccccctgcctgtgccccgggtgaccctgccggctccggcgag |
| | | | ggcaggggaagtctactaacatgcggggacgtggaggaaaatcccggcccagcctcctccgagaacg |
| | | | tcatcaccgagttcatgcgcttcaaggtgcgcatggagggcaccgtgaacggccacgagttcgagat |
| | | | cgagggcgagggcgagggccgcccctacgagggccacaacaccgtgaagctgaaggtgaccaagggc |
| | | | ggccccctgcccttcgcctgggacatcctgtccccccagttccagtacggctccaaggtgtacgtga |
| | | | agcaccccgccgacatccccgactacaagaagctgtccttccccgagggcttcaagtgggagcgcgt |
| | | | gatgaacttcgaggacggcggcgtggcgaccgtgacccaggactcctccctgcaggacggctgcttc |
| | | | atctacaaggtgaagttcatcggcgtgaacttcccctccgacggccccgtgatgcagaagaagacca |
| | | | tgggctgggaggcctccaccgagcgcctgtacccccgcgacggcgtgctgaagggcgagacccacaa |
| | | | ggccctgaagctgaaggacggcggccactacctggtggagttcaagtccatctacatggccaagaag |
| | | | cccgtgcagctgcccggctactactacgtggacgccaagctggacatcacctcccacaacgaggact |
| | | | acaccatcgtggagcagtacgagcgcaccgagggccgccaccacctgttcctgtag |
| LL056 | plasmid | TRB pro cpf1 | TCAActacaggaacaggtggtggcggccctcggtgcgctcgtactgctccacgatggtgtagtcctc |
| | | RFP tefris | gttgtgggaggtgatgtccagcttggcgtccacgtagtagtagccgggcagctgcacgggcttcttg |
| | | donor | gccatgtagatggacttgaactccaccaggtagtggccgccgtccttcagcttcagggccttgtggg |
| | | antisense | tctcgcccttcagcacgccgtcgcgggggtacaggcgctcggtggaggcctcccagcccatggtctt |
| | | | cttctgcatcacggggccgtcggaggggaagttcacgccgatgaacttcaccttgtagatgaagcag |
| | | | ccgtcctgcagggaggagtcctgggtcacggtcgccacgccgccgtcctcgaagttcatcacgcgct |
| | | | cccacttgaagccctcggggaaggacagcttcttgtagtcggggatgtcggcggggtgcttcacgta |
| | | | caccttggagccgtactggaactggggggacaggatgtcccaggcgaagggcagggggccgcccttg |
| | | | gtcaccttcagcttcacggtgttgtggccctcgtaggggcggccctcgccctcgccctcgatctcga |
| | | | actcgtggccgttcacggtgccctccatgcgcaccttgaagcgcatgaactcggtgatgacgttctc |
| | | | ggaggaggctgggccgggattttcctccacgtccccgcatgttagtagacttcccctgccctcgccg |
| | | | gagccggcagggtcacccggggcacaggcagggggaccctgtgtctccctgctctgtgtccttgtcg |
| | | | tctgggtgtgcttccctggccctgagacaggggctgcttgggaagtctctgctcctgttggtgtcac |
| | | | tattagtgtctacagctcacacttcctctttcctttccaagcttggcactggcacc |
| LL057 | dsDNA | TRAC exon1 | TCCCATTCTGCTAATGCCCAGCCTAAGTTGGGGAGACCACTCCAGATTCCA |
| | | TALEN sfGFP | AGATGTACAGTTTGCTTTGCTGGGCCTTTTTCCCATGCCTGCCTTTACTCTG |
| | | donor | CCAGAGTTATATTGCTGGGGTTTTGAAGAAGATCCTATTAAATAAAAGAATA |
| | | | AGCAGTATTATTAAGTAGCCCTGCATTTCAGGTTTCCTTGAGTGGCAGGCC |
| | | | AGGCCTGGCCGTGAACGTTCACTGAAATCATGGCCTCTTGGCCAAGATTGA |
| | | | TAGCTTGTGCCTGTCCCTGAGTCCCAGTCCATCACGAGCAGCTGGTTTCTA |
| | | | AGATGCTATTTCCCGTATAAAGCATGAGACCGTGACTTGCCAGCCCCACAG |
| | | | AGCCCCGCCCTTGTCCATCACTGGCATCTGGACTCCAGCCTGGGTTGGGG |
| | | | CAAAGAGGGAAATGAGATCATGTCCTAACCCTGATCCTCTTGTCCCACAGA |
| | | | TATCCAGAACCCTGACCCTGCCGTGGGCTCCGGCGAGGGCAGGGGAAGT |
| | | | CTACTAACATGCGGGGACGTGGAGGAAAATCCCGGCCCAAGCAAAGGAGA |
| | | | AGAACTTTTCACTGGAGTTGTCCCAATTCTTGTTGAATTAGATGGTGATGTT |
| | | | AATGGGCACAAATTTTCTGTCCGTGGAGAGGGTGAAGGTGATGCTACAAAC |
| | | | GGAAAACTCACCCTTAAATTTATTTGCACTACTGGAAAACTACCTGTTCCGT |
| | | | GGCCAACACTTGTCACTACTCTGACCTATGGTGTTCAATGCTTTTCCCGTTA |
| | | | TCCGGATCACATGAAACGGCATGACTTTTTCAAGAGTGCCATGCCCGAAGG |
| | | | TTATGTACAGGAACGCACTATATCTTTCAAAGATGACGGGACCTACAAGAC |
| | | | GCGTGCTGAAGTCAAGTTTGAAGGTGATACCCTTGTTAATCGTATCGAGTTA |
| | | | AAGGGTATTGATTTTAAAGAAGATGGAAACATTCTTGGACACAAACTCGAGT |
| | | | ACAACTTTAACTCACACAATGTATACATCACGGCAGACAAACAAAAGAATGG |
| | | | AATCAAAGCTAACTTCAAAATTCGCCACAACGTTGAAGATGGTTCCGTTCAA |
| | | | CTAGCAGACCATTATCAACAAAATACTCCAATTGGCGATGGCCCTGTCCTTT |
| | | | TACCAGACAACCATTACCTGTCGACACAATCTGTCCTTTCGAAAGATCCCAA |
| | | | CGAAAAGCGTGACCACATGGTCCTTCTTGAGTTTGTAACTGCTGCTGGGAT |
| | | | TACACATGGCATGGATGAGCTCTACAAATAATAGCACCGATTTTGATTCTCA |
| | | | AACAAATGTGTCACAAAGTAAGGATTCTGATGTGTATATCACAGACAAAACT |
| | | | GTGCTAGACATGAGGTCTATGGACTTCAAGAGCAACAGTGCTGTGGCCTG |
| | | | GAGCAACAAATCTGACTTTGCATGTGCAAACGCCTTCAACAACAGCATTATT |
| | | | CCAGAAGACACCTTCTTCCCCAGCCCAGGTAAGGGCAGCTTTGGTGCCTTC |
| | | | GCAGGCTGTTTCCTTGCTTCAGGAATGGCCAGGTTCTGCCCAGAGCTCTG |
| | | | GTCAATGATGTCTAAAACTCCTCTGATTGGTGGTCTCGGCCTTATCCATTGC |
| | | | CACCAAAACCCTCTTTTTACTAAGAAACAGTGAGCCTTGTTCTGGCAGTCCA |
| | | | GAGAATGACACGGGAAAAAAGCAGATGAAGAGAAGGTGGCAGGAGAGGG |
| | | | CACGTGGCCCAGCCTCAGTCTCTCCAACTGAGTTCCTGCCTGCCTGCCTTT |
| | | | GCTCAGACTGTTTGCCCCTTAC |
| LL058 | dsDNA | TRA pro | TTAACTCAACTCACTGCAACCCACTGCAACCCCTGACATAACCAATGTTAGT |
| | | TALEN 147p- | AGTTTATTGAGTATATCCTCACACTTTTAAAAATGTATGCATATGTACATAAG |
| | | sgGFP donor | TTTATGATAAAAATATCATTCAATACTCATCACTCTGCAACTTACTTTTGAATA |
| | | | TATTAAAGATTATTTCTATATTAGCTGTTGTAAGCACACTTAAATGGTAGGTA |
| | | | AATTTCCTTGTCTTTCTAGCTTCCAAAATATATATGACACACAAACAAACAAT |
| | | | ATTTAGTATATGCACACACACACTGCATCTCTAATTGATCCTGGATTTCATTT |
| | | | TGTTGTATCATGAGAAAGAAAACTGTTAGTATGGTCAAATTGATTAGTTTTGA |
| | | | CTTTGCCTTATGTTCCCATTTGTTTTCTCTGTTCTTTACATGTTCGATGTTCA |
| | | | CCATAATCACTTGGATTAAAATGTGTGGATTAGTTTTTGGAGAAGTCACCCA |
| | | | AGTGTGGTCTAATATAAATCCTGTGTTCCTGAGGTCATGCAGATTGAGAGA |
| | | | GGAAGTGATGTCACTGTGGGAACTTCCGTGTAAGGACGGGGCGTCCCTCC |
| | | | TCCTCTGCTCCTGCTCACAGTGATCCTGATCTGGTAAGAGCTCCCATCCTG |
| | | | CCCTGACCCTGCCATGAGCAAGGGCGAGGAGCTGTTCACCGGGGTGGTG |
| | | | CCCATCCTGGTCGAGCTGGACGGCGACGTAAACGGCCACAAGTTCAGCGT |
| | | | GCGTGGCGAGGGCGAGGGCGATGCCACCAACGGCAAGCTGACCCTGAAG |
| | | | TTCATCTGCACCACCGGCAAGCTGCCCGTGCCCTGGCCCACCCTCGTGAC |
| | | | CACCCTGACCTACGGCGTGCAGTGCTTCAGCCGCTACCCCGACCACATGA |
| | | | AGCGTCACGACTTCTTCAAGTCCGCCATGCCCGAAGGCTACGTCCAGGAG |
| | | | CGCACCATCTCGTTCAAGGACGACGGCACATACAAGACCCGCGCCGAGGT |
| | | | GAAGTTCGAGGGCGACACCCTGGTGAACCGCATCGAGCTGAAGGGCATCG |
| | | | ACTTCAAGGAGGACGGCAACATCCTGGGGCACAAGCTGGAGTACAACTTTA |
| | | | ACAGCCACAACGTCTATATCACAGCCGACAAGCAGAAGAACGGCATCAAG |
| | | | GCAAACTTCAAGATCCGCCACAACGTTGAGGACGGCAGCGTGCAGCTCGC |
| | | | CGACCACTACCAGCAGAACACCCCCATCGGCGACGGCCCCGTGCTGCTGC |
| | | | CCGACAACCACTACCTGAGCACCCAGTCCGTTCTGAGCAAAGACCCCAAC |
| | | | GAGAAGCGCGATCACATGGTCCTGCTGGAGTTCGTGACCGCCGCCGGGAT |
| | | | CACTCACGGCATGGACGAGCTGTACAAGTAATAGGCCTCAAGGGATTCTTC |
| | | | TACCTCAGCGTCTTGAGTAGCTGGGATTACAGGCATAAGCCACTGTGCCCA |
| | | | GCTTAAAACCTGTGGATTTATCAGTAGAAAATGTTCATGTAAAGATACTCCT |
| | | | GTAAGAGAAACCATAGCTGCTCCAGTGGAAGGAAGCTTAAACTCATCCCTT |
| | | | CAAGAAAGAAGCTCCTCCCTTTGTATTTCTACTGGGTTTTGCATCCGGACTG |
| | | | ATCTTCCTTCCCTCACCCACATGAAGTGTCTACCTTCTGCAGACTACAGTGG |
| | | | CTCAGGAACCGGGGATGCAGTGCCAGGCTCATGGTATCCTGCAGCAGATG |
| | | | TGGGGAGCTTTCCTTCTCTATGTTTCCATGAAGATGGGAGGTGAGTCTCAA |
| | | | TCTAATAGTAAATGCTGCTAGGAATTTT |
| LL059 | plasmid | TRB exon1 | TGTGTCACTACCCCACGAGACAAATATATACATCTTGATTTAAAAAAGGAAA |
| | | TALEN dsRed2 | ATTATAATTAGAAAAAGTCAATTTAGTTATTGTAATTATACCACTAATGAGAGT |
| | | donor | TTCCTACCTCGAGTTTCAGGATTACATAGCCATGCACCAAGCAAGGCTTTGA |
| | | | AAAATAAAGATACACAGATAAATTATTTGGATAGATGATCAGACAAGCCTCA |
| | | | GTAAAAACAGCCAAGACAATCAGGATATAATGTGACCATAGGAAGCTGGGG |
| | | | AGACAGTAGGCAATGTGCATCCATGGGACAGCATAGAAAGGAGGGGCAAA |
| | | | GTGGAGAGAGAGCAACAGACACTGGGATGGTGACCCCAAAACAATGAGGG |
| | | | CCTAGAATGACATAGTTGTGCTTCATTACGGCCCATTCCCAGGGCTCTCTC |
| | | | TCACACACACAGAGCCCCTACCAGAACCAGACAGCTCTCAGAGCAACCCT |
| | | | GGCTCCAACCCCTCTTCCCTTTCCAGAGGACCTGAACGGCTCCGGCGAGG |
| | | | GCAGGGGAAGTCTACTAACATGCGGGGACGTGGAGGAAAATCCCGGCCCA |
| | | | GCCTCCTCCGAGAACGTCATCACCGAGTTCATGCGCTTCAAGGTGCGCAT |
| | | | GGAGGGCACCGTGAACGGCCACGAGTTCGAGATCGAGGGCGAGGGCGAG |
| | | | GGCCGCCCCTACGAGGGCCACAACACCGTGAAGCTGAAGGTGACCAAGG |
| | | | GCGGCCCCCTGCCCTTCGCCTGGGACATCCTGTCCCCCCAGTTCCAGTAC |
| | | | GGCTCCAAGGTGTACGTGAAGCACCCCGCCGACATCCCCGACTACAAGAA |
| | | | GCTGTCCTTCCCCGAGGGCTTCAAGTGGGAGCGCGTGATGAACTTCGAGG |
| | | | ACGGCGGCGTGGCGACCGTGACCCAGGACTCCTCCCTGCAGGACGGCTG |
| | | | CTTCATCTACAAGGTGAAGTTCATCGGCGTGAACTTCCCCTCCGACGGCCC |
| | | | CGTGATGCAGAAGAAGACCATGGGCTGGGAGGCCTCCACCGAGCGCCTG |
| | | | TACCCCCGCGACGGCGTGCTGAAGGGCGAGACCCACAAGGCCCTGAAGC |
| | | | TGAAGGACGGCGGCCACTACCTGGTGGAGTTCAAGTCCATCTACATGGCC |
| | | | AAGAAGCCCGTGCAGCTGCCCGGCTACTACTACGTGGACGCCAAGCTGGA |
| | | | CATCACCTCCCACAACGAGGACTACACCATCGTGGAGCAGTACGAGCGCA |
| | | | CCGAGGGCCGCCACCACCTGTTCCTGTAGCCTCGGGTAAGTAAGCCCTTC |
| | | | CTTTTCCTCTCCCTCTCTCATGGTTCTTGACCTAGAACCAAGGCATGAAGAA |
| | | | CTCACAGACACTGGAGGGTGGAGGGTGGGAGAGACCAGAGCTACCTGTG |
| | | | CACAGGTACCCACCTGTCCTTCCTCCGTGCCAACAGTGTCCTACCAGCAAG |
| | | | GGGTCCTGTCTGCCACCATCCTCTATGAGATCCTGCTAGGGAAGGCCACC |
| | | | CTGTATGCTGTGCTGGTCAGCGCCCTTGTGTTGATGGCCATGGTAAGCAG |
| | | | GAGGGCAGGATGGGGCCAGCAGGCTGGAGGTGACACACTGACACCAAGC |
| | | | ACCCAGAAGTATAGAGTCCCTGCCAGGATTGGAGCTGGGCAGTAGGGAGG |
| | | | GAAGAGATTTCATTCAGGTGCCTCAGAAGATAACTTGCACCTCTGTAGGAT |
| | | | CACAGTGGAAGGGTCATGCTGGGAAGGAGAAGCTGGAGTCACCAGAAAAC |
| | | | CCAATGGATGTTGTGATGAGCCTTAC |
| LL060 | dsDNA | TRB pro | CTTAAAATGATGCACAGCTGGCTCCAGGGAAGGGCTCCACTGAGCTAGGT |
| | | TALEN pro147 | GAGGTGTCCTCCTGGAATTCACTGAGATGAGGGAGGGGAGCTGGAGTGTG |
| | | dsRed2 donor | CTCATCCTGGGTCCAAGACAGGCATCGGGAAGGCATCTGCCCAAAGGGAA |
| | | | GGGGTCTGTGTGTTAGGGAGGAGGGGAGCCATAAGTAGAAAGAGGAAGG |
| | | | GGAGACCCATTCATTCGTTGTGGGAAGGGCAGGCAGCTGCTAAGAAAAAA |
| | | | GCAACTGTCTAAAGAACCCGCCCTGCACACCTGGCCCTGAGAAGCTAGTCT |
| | | | AAACCCACCTCTTGAGGTGCCAGTGCCAAGCTTGGAAAGGAAAGAGGAAG |
| | | | TGTGAGCTGTAGACACTAATAGTGACACCAACAGGAGCAGAGACTTCCCAA |
| | | | GCAGCCCCTGTCTCAGTCACCCAAGTGTGGTCTAATATAAATCCTGTGTTC |
| | | | CTGAGGTCATGCAGATTGAGAGAGGAAGTGATGTCACTGTGGGAACTTCC |
| | | | GTGTAAGGACGGGGCGTCCCTCCTCCTCTGCTCCTGCTCACAGTGATCCT |
| | | | GATCTGGTAAGAGCTCCCATCCTGCCCTGACCCTGCCATGGCCTCCTCCG |
| | | | AGAACGTCATCACCGAGTTCATGCGCTTCAAGGTGCGCATGGAGGGCACC |
| | | | GTGAACGGCCACGAGTTCGAGATCGAGGGCGAGGGCGAGGGCCGCCCCT |
| | | | ACGAGGGCCACAACACCGTGAAGCTGAAGGTGACCAAGGGCGGCCCCCT |
| | | | GCCCTTCGCCTGGGACATCCTGTCCCCCCAGTTCCAGTACGGCTCCAAGG |
| | | | TGTACGTGAAGCACCCCGCCGACATCCCCGACTACAAGAAGCTGTCCTTCC |
| | | | CCGAGGGCTTCAAGTGGGAGCGCGTGATGAACTTCGAGGACGGCGGCGT |
| | | | GGCGACCGTGACCCAGGACTCCTCCCTGCAGGACGGCTGCTTCATCTACA |
| | | | AGGTGAAGTTCATCGGCGTGAACTTCCCCTCCGACGGCCCCGTGATGCAG |
| | | | AAGAAGACCATGGGCTGGGAGGCCTCCACCGAGCGCCTGTACCCCCGCG |
| | | | ACGGCGTGCTGAAGGGCGAGACCCACAAGGCCCTGAAGCTGAAGGACGG |
| | | | CGGCCACTACCTGGTGGAGTTCAAGTCCATCTACATGGCCAAGAAGCCCG |
| | | | TGCAGCTGCCCGGCTACTACTACGTGGACGCCAAGCTGGACATCACCTCC |
| | | | CACAACGAGGACTACACCATCGTGGAGCAGTACGAGCGCACCGAGGGCC |
| | | | GCCACCACCTGTTCCTGTAGGCAGGGAGACACAGGGTCCCCCTGCCTGTG |
| | | | CCCCGGGTGACCCTGCCATGGGCTGAAGTCTCCACTGTGGTGTGGTCCAT |
| | | | TGTCTCAGGTGAGTCCTGGGCACAGGTGGGACATTTCTGTCCTTAAATTTTT |
| | | | TGCTTTTTTCATGGAACTGCTTCAGAAGATTCTGTCCTAGGCTTAGTCTGAA |
| | | | TTTGGCTTCTTATTTTCATAGGCTCCATGGATACTGGAATTACCCAGACACC |
| | | | AAAATACCTGGTCACAGCAATGGGGAGTAAAAGGACAATGAAACGTGAGCA |
| | | | TCTGGGACATGATTCTATGTATTGGTACAGACAGAAAGCTAAGAAATCCCTG |
| | | | GAGTTCATGTTTTACTACAACTGTAAGGAATTCATTGAAAACAAGACTGTGC |
| | | | CAAATCACTT |
| LL061 | | TRAC pro | cactgcatctctaattgatcctggatttcattttgttgtatcatgagaaagaaaactgttagtatgg |
| | | nickase pro- | tcaaattgattagttttgactttgccttatgttcccatttgttttctctgttctttacatgttcgat |
| | | 147 sfGFP | gttcaccataatcacttggattaaaatgtgtggattagtttttggagatagggacctcaccatgttg |
| | | donor | cttaggctggtctccagttcctggcctcaagggattcttctacctcagcgtcttgagtagctgggat |
| | | | tacaggcataagccactgtgcccagcttaaaacctgtggatttatcagtagaaaatgttcatgtaaa |
| | | | gatactcctgtaagagaaaccatagctgctccagtggaaggaagcttaaactcatcccttcaagaaa |
| | | | gaagctcctccctttgtatttctactgggttttgcatccggactgatcttccttccctcacccacat |
| | | | gaagtgtctaccttctagtcacccaagtgtggtctaatataaatcctgtgttcctgaggtcatgcag |
| | | | attgagagaggaagtgatgtcactgtgggaacttccgtgtaaggacggggcgtccctcctcctctgc |
| | | | tcctgctcacagtgatcctgatctggtaaGagctcccatcctgccctgaccctgccatgagcaaggg |
| | | | cgaggagctgttcaccggggtggtgcccatcctggtcgagctggacggcgacgtaaacggccacaag |
| | | | ttcagcgtgcgtggcgagggcgagggcgatgccaccaacggcaagctgaccctgaagttcatctgca |
| | | | ccaccggcaagctgcccgtgccctggcccaccctcgtgaccaccctgacctacggcgtgcagtgctt |
| | | | cagccgctaccccgaccacatgaagcgtcacgacttcttcaagtccgccatgcccgaaggctacgtc |
| | | | caggagcgcaccatctcgttcaaggacgacggcacatacaagacccgcgccgaggtgaagttcgagg |
| | | | gcgacaccctggtgaaccgcatcgagctgaagggcatcgacttcaaggaggacggcaacatcctggg |
| | | | gcacaagctggagtacaactttaacagccacaacgtctatatcacagccgacaagcagaagaacggc |
| | | | atcaaggcaaacttcaagatccgccacaacgttgaggacggcagcgtgcagctcgccgaccactacc |
| | | | agcagaacacccccatcggcgacggccccgtgctgctgcccgacaaccactacctgagcacccagtc |
| | | | cgttctgagcaaagaccccaacgagaagcgcgatcacatggtcctgctggagttcgtgaccgccgcc |
| | | | gggatcactcacggcatggacgagctgtacaagTAATAGgccaggctcatggtatcctgcagcagat |
| | | | gtggggagctttccttctctatgtttccatgaagatgggaggtgagtctcaatctaatagtaaatgc |
| | | | tgctaggaattttcaaaacaatttcctttcagctaaattattgcaaattttgacatttgtaatgaga |
| | | | gtatttcctgaatatgcattttcctaacgtggtgctaattgtcctcctgttactattgctgctgctg |
| | | | ttactgcaaccatttatttcagtctaagaaattctcccatcaatggcagttcttttgtgaccacatg |
| | | | gaagcatcatttaaaaaattattccaatagtttttggaggaaacatcatttttaataatgatggggc |
| | | | ttctgggggtgctgccctagtaacaatcatgtatcttgtcataggcactgcaggacaaagccttgag |
| | | | ca |
| LL062 | oligos | TRAC primer | CTGAGTCCCAGTCCATCACGA |
| | (primer) | | |
| LL063 | oligos | TRAC primer | CGAGACCACCAATCAGAGGAG |
| | (primer) | | |
| LL064 | oligos | TRAC primer | TGGCCAAGATTGATAGCTTGT |
| | (primer) | | |
| LL065 | oligos | TRAC primer | GCCACCTTCTCTTCATCTGC |
| | (primer) | | |
| LL066 | | | gccttatatcgagtaaacggtagtgctggggcttagacgcaggtgttctgatttatagttcaaaacc |
| | | | tctatcaatgagagagcaatctcctggtaatgtgatagatttcccaacttaatgccaacataccata |
| | | | aacctcccattctgctaatgcccagcctaagttggggagaccactccagattccaagatgtacagtt |
| | | | tgctttgctgggcctttttcccatgcctgcctttactctgccagagttatattgctggggttttgaa |
| | | | gaagatcctattaaataaaagaataagcagtattattaagtagccctgcatttcaggtttccttgag |
| | | | tggcaggccaggcctggccgtgaacgttcactgaaatcatggcctcttggccaagattgatagcttg |
| | | | tgcctgtccctgagtcccagtccatcacgagcagctggtttctaagatgctatttcccgtataaagc |
| | | | atgagaccgtgacttgccagccccacagagccccgcccttgtccatcactggcatctggactccagc |
| | | | ctgggttggggcaaagagggaaatgagatcatgtcctaaccctgatcctcttgtcccacagatatcc |
| | | | agaaccctgaccctgccgtgtaccagctgagagactctaaatccagtgacaagtctgtctgcctagg |
| | | | ctccggcgagggcaggggaagtctactaacatgcggggacgtggaggaaaatcccggcccaagcaaa |
| | | | ggaga |
| LL067 | | | ctactaacatgcggggacgtggaggaaaatcccggcccaagcaaaggagaagaacttttcactggag |
| | | | ttgtcccaattcttgttgaattagatggtgatgttaatgggcacaaattttctgtccgtggagaggg |
| | | | tgaaggtgatgctacaaacggaaaactcacccttaaatttatttgcactactggaaaactacctgtt |
| | | | ccgtggccaacacttgtcactactctgacctatggtgttcaatgcttttcccgttatccggatcaca |
| | | | tgaaacggcatgactttttcaagagtgccatgcccgaaggttatgtacaggaacgcactatatcttt |
| | | | caaagatgacgggacctacaagacgcgtgctgaagtcaagtttgaaggtgatacccttgttaatcgt |
| | | | atcgagttaaagggtattgattttaaagaagatggaaacattcttggacacaaactcgagtacaact |
| | | | ttaactcacacaatgtatacatcacggcagacaaacaaaagaatggaatcaaagctaacttcaaaat |
| | | | tcgccacaacgttgaagatggttccgttcaactagcagaccattatcaacaaaatactccaattggc |
| | | | gatggccctgtccttttaccagacaaccattacctgtcgacacaatctgtcctttcgaaagatccca |
| | | | acgaaaagcgtgaccacatggtccttcttgagtttgtaactgctgctgggattacacatggcatgga |
| | | | tgagctctacaaa |
| LL068 | | | agtttgtaactgctgctgggattacacatggcatggatgagctctacaaaTAATAGaccgattttga |
| | | | ttctcaaacaaatgtgtcacaaagtaaggattctgatgtgtatatcacagacaaaactgtgctagac |
| | | | atgaggtctatggacttcaagagcaacagtgctgtggcctggagcaacaaatctgactttgcatgtg |
| | | | caaacgccttcaacaacagcattattccagaagacaccttcttccccagcccaggtaagggcagatt |
| | | | ggtgccttcgcaggctgtttccttgcttcaggaatggccaggttctgcccagagctctggtcaatga |
| | | | tgtctaaaactcctctgattggtggtctcggccttatccattgccaccaaaaccctctttttactaa |
| | | | gaaacagtgagccttgttctggcagtccagagaatgacacgggaaaaaagcagatgaagagaaggtg |
| | | | gcaggagagggcacgtggcccagcctcagtctctccaactgagttcctgcctgcctgcctttgctca |
| | | | gactgtttgccccttactgctcttctaggcctcattctaagccccttctccaagttgcctctcctta |
| | | | tttctccctgtctgccaaaaaatctttcccagctcactaagtcagtctcacgcagtcactcattaac |
| | | | ccaccaatcactgattgtgccggcacatgaatgcaccaggtgttgaagtggaggaattaaaaagtca |
| | | | gatgaggggtg |
| LL069 | ssDNA | Trac exon1 | actccagattccaagatgtacagtttgctttgctgggcctttttcccatgcctgcctttactctgcc |
| | | Cas9 HDR | agagttatattgctggggttttgaagaagatcctattaaataaaagaataagcagtattattaagta |
| | | iRFP donor | gccctgcatttcaggtttccttgagtggcaggccaggcctggccgtgaacgttcactgaaatcatgg |
| | | | cctcttggccaagattgatagcttgtgcctgtccctgagtcccagtccatcacgagcagctggtttc |
| | | | taagatgctatttcccgtataaagcatgagaccgtgacttgccagccccacagagccccgcccttgt |
| | | | ccatcactggcatctggactccagcctgggttggggcaaagagggaaatgagatcatgtcctaaccc |
| | | | tgatcctcttgtcccacagatatccagaaccctgaccctgccgtgtaccagctgagagactctaaat |
| | | | ccagtgacaagtctgtctgcctaggctccggcgagggcaggggaagtctactaacatgcggggacgt |
| | | | ggaggaaaatcccggcccagctgaaggatccgtcgccaggcagcctgacctcttgacctgcgacgat |
| | | | gagccgatccatatccccggtgccatccaaccgcatggactgctgctcgccctcgccgccgacatga |
| | | | cgatcgttgccggcagcgacaaccttcccgaactcaccggactggcgatcggcgccctgatcggccg |
| | | | ctctgcggccgatgtcttcgactcggagacgcacaaccgtctgacgatcgccttggccgagcccggg |
| | | | gcggccgtcggagcaccgatcactgtcggcttcacgatgcgaaaggacgcaggcttcatcggctcct |
| | | | ggcatcgccatgatcagctcatcttcctcgagctcgagcctccccagcgggacgtcgccgagccgca |
| | | | ggcgttcttccgccgcaccaacagcgccatccgccgcctgcaggccgccgaaaccttggaaagcgcc |
| | | | tgcgccgccgcggcgcaagaggtgcggaagattaccggcttcgatcgggtgatgatctatcgcttcg |
| | | | cctccgacttcagcggcgaagtgatcgcagaggatcggtgcgccgaggtcgagtcaaaactaggcct |
| | | | gcactatcctgcctcaaccgtgccggcgcaggcccgtcggctctataccatcaacccggtacggatc |
| | | | attcccgatatcaattatcggccggtgccggtcaccccagacctcaatccggtcaccgggcggccga |
| | | | ttgatcttagcttcgccatcctgcgcagcgtctcgcccgtccatctggaattcatgcgcaacatagg |
| | | | catgcacggcacgatgtcgatctcgattttgcgcggcgagcgactgtggggattgatcgtttgccat |
| | | | caccgaacgccgtactacgtcgatctcgatggccgccaagcctgcgagctagtcgcccaggttctgg |
| | | | cctggcagatcggcgtgatggaagagtgaTAGaccgattttgattctcaaacaaatgtgtcacaaag |
| | | | taaggattctgatgtgtatatcacagacaaaactgtgctagacatgaggtctatggacttcaagagc |
| | | | aacagtgctgtggcctggagcaacaaatctgactttgcatgtgcaaacgccttcaacaacagcatta |
| | | | ttccagaagacaccttcttccccagcccaggtaagggcagctttggtgccttcgcaggctgtttcct |
| | | | tgcttcaggaatggccaggttctgcccagagctctggtcaatgatgtctaaaactcctctgattggt |
| | | | ggtctcggccttatccattgccaccaaaaccctctttttactaagaaacagtgagccttgttctggc |
| | | | agtccagagaatgacacgggaaaaaagcagatgaagagaaggtggcaggagagggcacgtggcccag |
| | | | cctcagtctctccaactgagttcctgcctgcctgcctttgctcagactgtttgc |
| LL070 | ssDNA, | Trac exon1 | actccagattccaagatgtacagtttgctttgctgggcctttttcccatgcctgcctttactctgcc |
| | plasmid | Cas9 HDR | agagttatattgctggggttttgaagaagatcctattaaataaaagaataagcagtattattaagta |
| | | rLuc donor | gccctgcatttcaggtttccttgagtggcaggccaggcctggccgtgaacgttcactgaaatcatgg |
| | | | cctcttggccaagattgatagcttgtgcctgtccctgagtcccagtccatcacgagcagctggtttc |
| | | | taagatgctatttcccgtataaagcatgagaccgtgacttgccagccccacagagccccgcccttgt |
| | | | ccatcactggcatctggactccagcctgggttggggcaaagagggaaatgagatcatgtcctaaccc |
| | | | tgatcctcttgtcccacagatatccagaaccctgaccctgccgtgtaccagctgagagactctaaat |
| | | | ccagtgacaagtctgtctgcctaggctccggcgagggcaggggaagtctactaacatgcggggacgt |
| | | | ggaggaaaatcccggcccaGAAAATATGGAAAACGACGAGAACATCGTGGTGGGCCCCA |
| | | | AGCCCTTCTACCCCATCGAGGAAGGCAGCGCCGGCACCCAGCTGCGGAAG |
| | | | TACATGGAAAGATACGCCAAGCTGGGCGCCATTGCCTTCACCAACGCCGTG |
| | | | ACCGGCGTGGACTACAGCTACGCCGAGTACCTGGAAAAGAGCTGCTGCCT |
| | | | GGGCAAGGCTCTGCAGAACTACGGCCTGGTGGTGGACGGCCGGATCGCCC |
| | | | TGTGCAGCGAGAACTGCGAGGAATTCTTCATCCCCGTGATCGCCGGCCTGT |
| | | | TCATCGGCGTGGGCGTGGCTCCCACCAACGAGATCTACACCCTGCGGGAG |
| | | | CTGGTGCACAGCCTGGGCATCAGCAAGCCCACCATCGTGTTCAGCAGCAAG |
| | | | AAGGGCCTGGACAAAGTCATCACCGTGCAGAAAACCGTGACCACCATCAAG |
| | | | ACCATCGTGATCCTGGACAGCAAGGTGGACTACCGGGGCTACCAGTGCCT |
| | | | GGACACCTTCATCAAGCGGAACACCCCCCCTGGCTTCCAGGCCAGCAGCTT |
| | | | CAAGACCGTGGAGGTGGACCGGAAAGAACAGGTGGCCCTGATCATGAACA |
| | | | GCAGCGGCAGCACCGGCCTGCCCAAGGGCGTGCAGCTGACCCACGAGAA |
| | | | CACCGTGACCCGGTTCAGCCACGCCAGGGACCCCATCTACGGCAACCAGG |
| | | | TGTCCCCCGGCACCGCCGTGCTGACCGTGGTGCCCTTCCACCACGGCTTC |
| | | | GGCATGTTCACCACCCTGGGCTACCTGATCTGCGGCTTCCGGGTGGTGATG |
| | | | CTGACCAAGTTCGACGAGGAAACCTTCCTGAAAACCCTGCAGGACTACAAG |
| | | | TGCACCTACGTGATTCTGGTGCCCACCCTGTTCGCCATCCTGAACAAGAGC |
| | | | GAGCTGCTGAACAAGTACGACCTGAGCAACCTGGTGGAGATCGCCAGCGG |
| | | | CGGAGCCCCCCTGAGCAAAGAAGTGGGAGAGGCCGTCGCCAGGCGGTTCA |
| | | | ATCTGCCCGGCGTGCGGCAGGGCTACGGCCTGACCGAGACAACCAGCGCC |
| | | | ATCATCATCACCCCCGAGGGCGACGACAAGCCTGGAGCCAGCGGCAAGGT |
| | | | GGTGCCCCTGTTCAAGGCCAAAGTGATCGACCTGGACACCAAGAAGAGCCT |
| | | | GGGCCCCAACAGACGGGGCGAAGTGTGCGTGAAGGGCCCCATGCTGATGA |
| | | | AGGGCTACGTGAACAACCCCGAGGCCACCAAAGAGCTGATCGACGAAGAG |
| | | | GGCTGGCTGCACACCGGCGACATCGGCTACTACGACGAAGAGAAGCACTT |
| | | | CTTCATCGTGGACCGGCTGAAGAGCCTGATCAAGTACAAGGGCTATCAGGT |
| | | | GCCCCCTGCCGAGCTGGAAAGCGTCCTGCTGCAGCACCCCAGCATCTTCG |
| | | | ACGCCGGCGTGGCCGGGGTGCCAGATCCTGTGGCCGGCGAGCTGCCTGG |
| | | | CGCCGTGGTGGTGCTGGAATCCGGCAAGAACATGACCGAGAAAGAAGTGA |
| | | | TGGACTACGTCGCCAGCCAGGTGTCCAACGCCAAGCGGCTGAGAGGCGGC |
| | | | GTGAGATTCGTGGACGAAGTGCCAAAGGGCCTGACCGGCAAGATCGACGG |
| | | | CAGGGCCATCCGGGAGATCCTGAAGAAACCCGTGGCCAAGATGTGATGAac |
| | | | cgattttgattctcaaacaaatgtgtcacaaagtaaggattctgatgtgtatatcacagacaaaact |
| | | | gtgctagacatgaggtctatggacttcaagagcaacagtgctgtggcctggagcaacaaatctgact |
| | | | ttgcatgtgcaaacgccttcaacaacagcattattccagaagacaccttcttccccagcccaggtaa |
| | | | gggcagctttggtgccttcgcaggctgtttccttgcttcaggaatggccaggttctgcccagagctc |
| | | | tggtcaatgatgtctaaaactcctctgattggtggtctcggccttatccattgccaccaaaaccctc |
| | | | tttttactaagaaacagtgagccttgttctggcagtccagagaatgacacgggaaaaaagcagatga |
| | | | agagaaggtggcaggagagggcacgtggcccagcctcagtctctccaactgagttcctgcctgcctg |
| | | | cctttgctcagactgtttgc |
| LL071 | plasmid, | 147bp | AGTCACCCAAGTGTGGTCTAATATAAATCCTGTGTTCCTGAGGTCATGCAGA |
| | dsDNA | TCR beta | TTGAGAGAGGAAGTGATGTCACTGTGGGAACTTCCGTGTAAGGACGGGGC |
| | (PCR), | promoter- | GTCCCTCCTCCTCTGCTCCTGCTCACAGTGATCCTGATCTGGTAAGAGCTC |
| | dsDNA | sfGFP- | CCATCCTGCCCTGACCCTGCCATGAGCAAGGGCGAGGAGCTGTTCACCGG |
| | | bGHpolyA | GGTGGTGCCCATCCTGGTCGAGCTGGACGGCGACGTAAACGGCCACAAGT |
| | | | TCAGCGTGCGTGGCGAGGGCGAGGGCGATGCCACCAACGGCAAGCTGAC |
| | | | CCTGAAGTTCATCTGCACCACCGGCAAGCTGCCCGTGCCCTGGCCCACCC |
| | | | TCGTGACCACCCTGACCTACGGCGTGCAGTGCTTCAGCCGCTACCCCGAC |
| | | | CACATGAAGCGTCACGACTTCTTCAAGTCCGCCATGCCCGAAGGCTACGTC |
| | | | CAGGAGCGCACCATCTCGTTCAAGGACGACGGCACATACAAGACCCGCGC |
| | | | CGAGGTGAAGTTCGAGGGCGACACCCTGGTGAACCGCATCGAGCTGAAG |
| | | | GGCATCGACTTCAAGGAGGACGGCAACATCCTGGGGCACAAGCTGGAGTA |
| | | | CAACTTTAACAGCCACAACGTCTATATCACAGCCGACAAGCAGAAGAACGG |
| | | | CATCAAGGCAAACTTCAAGATCCGCCACAACGTTGAGGACGGCAGCGTGC |
| | | | AGCTCGCCGACCACTACCAGCAGAACACCCCCATCGGCGACGGCCCCGT |
| | | | GCTGCTGCCCGACAACCACTACCTGAGCACCCAGTCCGTTCTGAGCAAAG |
| | | | ACCCCAACGAGAAGCGCGATCACATGGTCCTGCTGGAGTTCGTGACCGCC |
| | | | GCCGGGATCACTCACGGCATGGACGAGCTGTACAAGTAAGGATCTGCCTC |
| | | | GACTGTGCCTTCTAGTTGCCAGCCATCTGTTGTTTGCCCCTCCCCCGTGCC |
| | | | TTCCTTGACCCTGGAAGGTGCCACTCCCACTGTCCTTTCCTAATAAAATGAG |
| | | | GAAATTGCATCGCATTGTCTGAGTAGGTGTCATTCTATTCTGGGGGGTGGG |
| | | | GTGGGGCAGGACAGCAAGGGGGAGGATTGGGAAGACAATAGCAGGCATG |
| | | | CTGGGGATGCGGTGGGCTCTATGG |
| LL072 | plasmid, | 147bp | AGTCACCCAAGTGTGGTCTAATATAAATCCTGTGTTCCTGAGGTCATGCAGA |
| | dsDNA | TCRbeta | TTGAGAGAGGAAGTGATGTCACTGTGGGAACTTCCGTGTAAGGACGGGGC |
| | (PCR), | promoter- | GTCCCTCCTCCTCTGCTCCTGCTCACAGTGATCCTGATCTGGTAAGAGCTC |
| | dsDNA | dsRed2- | CCATCCTGCCCTGACCCTGCCATGGCCTCCTCCGAGAACGTCATCACCGA |
| | | bGHpolyA | GTTCATGCGCTTCAAGGTGCGCATGGAGGGCACCGTGAACGGCCACGAGT |
| | | | TCGAGATCGAGGGCGAGGGCGAGGGCCGCCCCTACGAGGGCCACAACAC |
| | | | CGTGAAGCTGAAGGTGACCAAGGGCGGCCCCCTGCCCTTCGCCTGGGAC |
| | | | ATCCTGTCCCCCCAGTTCCAGTACGGCTCCAAGGTGTACGTGAAGCACCC |
| | | | CGCCGACATCCCCGACTACAAGAAGCTGTCCTTCCCCGAGGGCTTCAAGT |
| | | | GGGAGCGCGTGATGAACTTCGAGGACGGCGGCGTGGCGACCGTGACCCA |
| | | | GGACTCCTCCCTGCAGGACGGCTGCTTCATCTACAAGGTGAAGTTCATCGG |
| | | | CGTGAACTTCCCCTCCGACGGCCCCGTGATGCAGAAGAAGACCATGGGCT |
| | | | GGGAGGCCTCCACCGAGCGCCTGTACCCCCGCGACGGCGTGCTGAAGGG |
| | | | CGAGACCCACAAGGCCCTGAAGCTGAAGGACGGCGGCCACTACCTGGTG |
| | | | GAGTTCAAGTCCATCTACATGGCCAAGAAGCCCGTGCAGCTGCCCGGCTA |
| | | | CTACTACGTGGACGCCAAGCTGGACATCACCTCCCACAACGAGGACTACA |
| | | | CCATCGTGGAGCAGTACGAGCGCACCGAGGGCCGCCACCACCTGTTCCTG |
| | | | TAGGGATCTGCCTCGACTGTGCCTTCTAGTTGCCAGCCATCTGTTGTTTGC |
| | | | CCCTCCCCCGTGCCTTCCTTGACCCTGGAAGGTGCCACTCCCACTGTCCTT |
| | | | TCCTAATAAAATGAGGAAATTGCATCGCATTGTCTGAGTAGGTGTCATTCTA |
| | | | TTCTGGGGGGTGGGGTGGGGCAGGACAGCAAGGGGGAGGATTGGGAAGA |
| | | | CAATAGCAGGCATGCTGGGGATGCGGTGGGCTCTATGG |
| LL073 | plasmid, | EF1a | gggcagagcgcacatcgcccacagtccccgagaagttggggggaggggtcggcaattgaaccggtgc |
| | dsDNA | promoter- | ctagagaaggtggcgcggggtaaactgggaaagtgatgtcgtgtactggctccgcctttttcccgag |
| | (PCR) | sfGFP- | ggtgggggagaaccgtatataagtgcagtagtcgccgtgaacgttctttttcgcaacgggtttgccg |
| | | bGHpolyA | ccagaacacaggtaagtgccgtgtgtggttcccgcgggcctggcctctttacgggttatggcccttg |
| | | | cgtgccttgaattacttccacctggctgcagtacgtgattcttgatcccgagcttcgggttggaagt |
| | | | gggtgggagagttcgaggccttgcgcttaaggagccccttcgcctcgtgcttgagttgaggcctggc |
| | | | ctgggcgctggggccgccgcgtgcgaatctggtggcaccttcgcgcctgtctcgctgctttcgataa |
| | | | gtctctagccatttaaaatttttgatgacctgctgcgacgctttttttctggcaagatagtcttgta |
| | | | aatgcgggccaagatctgcacactggtatttcggtttttggggccgcgggcggcgacggggcccgtg |
| | | | cgtcccagcgcacatgttcggcgaggcggggcctgcgagcgcggccaccgagaatcggacgggggta |
| | | | gtctcaagctggccggcctgctctggtgcctggcctcgcgccgccgtgtatcgccccgccctgggcg |
| | | | gcaaggctggcccggtcggcaccagttgcgtgagcggaaagatggccgcttcccggccctgctgcag |
| | | | ggagctcaaaatggaggacgcggcgctcgggagagcgggcgggtgagtcacccacacaaaggaaaag |
| | | | ggcctttccgtcctcagccgtcgcttcatgtgactccacggagtaccgggcgccgtccaggcacctc |
| | | | gattagttctcgcgcttttggagtacgtcgtctttaggttggggggaggggttttatgcgatggagt |
| | | | ttccccacactgagtgggtggagactgaagttaggccagcttggcacttgatgtaattctccttgga |
| | | | atttgccctttttgagtttggatcttggttcattctcaagcctcagacagtggttcaaagttttttt |
| | | | cttccatttcaggtgtcgtgagttaaatgagcaagggcgaggagctgttcaccggggtggtgcccat |
| | | | cctggtcgagctggacggcgacgtaaacggccacaagttcagcgtgcgtggcgagggcgagggcgat |
| | | | gccaccaacggcaagctgaccctgaagttcatctgcaccaccggcaagctgcccgtgccctggccca |
| | | | ccctcgtgaccaccctgacctacggcgtgcagtgcttcagccgctaccccgaccacatgaagcgtca |
| | | | cgacttcttcaagtccgccatgcccgaaggctacgtccaggagcgcaccatctcgttcaaggacgac |
| | | | ggcacatacaagacccgcgccgaggtgaagttcgagggcgacaccctggtgaaccgcatcgagctga |
| | | | agggcatcgacttcaaggaggacggcaacatcctggggcacaagctggagtacaactttaacagcca |
| | | | caacgtctatatcacagccgacaagcagaagaacggcatcaaggcaaacttcaagatccgccacaac |
| | | | gttgaggacggcagcgtgcagctcgccgaccactaccagcagaacacccccatcggcgacggccccg |
| | | | tgctgctgcccgacaaccactacctgagcacccagtccgttctgagcaaagaccccaacgagaagcg |
| | | | cgatcacatggtcctgctggagttcgtgaccgccgccgggatcactcacggcatggacgagctgtac |
| | | | aagtaaggatctgcctcgactgtgccttctagttgccagccatctgttgtttgcccctcccccgtgc |
| | | | cttccttgaccctggaaggtgccactcccactgtcctttcctaataaaatgaggaaattgcatcgca |
| | | | ttgtctgagtaggtgtcattctattctggggggtggggtggggcaggacagcaagggggaggattgg |
| | | | gaagacaatagcaggcatgctggggatgcggtgggctctatgg |
| LL074 | plasmid, | EF1a | gggcagagcgcacatcgcccacagtccccgagaagttggggggaggggtcggcaattgaaccggtgc |
| | dsDNA | promoter- | ctagagaaggtggcgcggggtaaactgggaaagtgatgtcgtgtactggctccgcctttttcccgag |
| | (PCR) | dsRed 2- | ggtgggggagaaccgtatataagtgcagtagtcgccgtgaacgttctttttcgcaacgggtttgccg |
| | | bGHpolyA | ccagaacacaggtaagtgccgtgtgtggttcccgcgggcctggcctctttacgggttatggcccttg |
| | | | cgtgccttgaattacttccacctggctgcagtacgtgattcttgatcccgagcttcgggttggaagt |
| | | | gggtgggagagttcgaggccttgcgcttaaggagccccttcgcctcgtgcttgagttgaggcctggc |
| | | | ctgggcgctggggccgccgcgtgcgaatctggtggcaccttcgcgcctgtctcgctgctttcgataa |
| | | | gtctctagccatttaaaatttttgatgacctgctgcgacgctttttttctggcaagatagtcttgta |
| | | | aatgcgggccaagatctgcacactggtatttcggtttttggggccgcgggcggcgacggggcccgtg |
| | | | cgtcccagcgcacatgttcggcgaggcggggcctgcgagcgcggccaccgagaatcggacgggggta |
| | | | gtctcaagctggccggcctgctctggtgcctggcctcgcgccgccgtgtatcgccccgccctgggcg |
| | | | gcaaggctggcccggtcggcaccagttgcgtgagcggaaagatggccgcttcccggccctgctgcag |
| | | | ggagctcaaaatggaggacgcggcgctcgggagagcgggcgggtgagtcacccacacaaaggaaaag |
| | | | ggcctttccgtcctcagccgtcgcttcatgtgactccacggagtaccgggcgccgtccaggcacctc |
| | | | gattagttctcgcgcttttggagtacgtcgtctttaggttggggggaggggttttatgcgatggagt |
| | | | ttccccacactgagtgggtggagactgaagttaggccagcttggcacttgatgtaattctccttgga |
| | | | atttgccctttttgagtttggatcttggttcattctcaagcctcagacagtggttcaaagttttttt |
| | | | cttccatttcaggtgtcgtgagttaaatggcctcctccgagaacgtcatcaccgagttcatgcgctt |
| | | | caaggtgcgcatggagggcaccgtgaacggccacgagttcgagatcgagggcgagggcgagggccgc |
| | | | ccctacgagggccacaacaccgtgaagctgaaggtgaccaagggcggccccctgcccttcgcctggg |
| | | | acatcctgtccccccagttccagtacggctccaaggtgtacgtgaagcaccccgccgacatccccga |
| | | | ctacaagaagctgtccttccccgagggcttcaagtgggagcgcgtgatgaacttcgaggacggcggc |
| | | | gtggcgaccgtgacccaggactcctccctgcaggacggctgcttcatctacaaggtgaagttcatcg |
| | | | gcgtgaacttcccctccgacggccccgtgatgcagaagaagaccatgggctgggaggcctccaccga |
| | | | gcgcctgtacccccgcgacggcgtgctgaagggcgagacccacaaggccctgaagctgaaggacggc |
| | | | ggccactacctggtggagttcaagtccatctacatggccaagaagcccgtgcagctgcccggctact |
| | | | actacgtggacgccaagctggacatcacctcccacaacgaggactacaccatcgtggagcagtacga |
| | | | gcgcaccgagggccgccaccacctgttcctgtagggatctgcctcgactgtgccttctagttgccag |
| | | | ccatctgttgtttgcccctcccccgtgccttccttgaccctggaaggtgccactcccactgtccttt |
| | | | cctaataaaatgaggaaattgcatcgcattgtctgagtaggtgtcattctattctggggggtggggt |
| | | | ggggcaggacagcaagggggaggattgggaagacaatagcaggcatgctggggatgcggtgggctct |
| | | | atgg |
| LL075 | oligos | LL027 | ggagaccactccagattcca |
| | (primer) | amplification | |
| LL076 | oligos | LL027 | cagtaaggggcaaacagtctga |
| | (primer) | amplification | |
| LL077 | oligos | LL028 | gaacgttcactgaaatcatgg |
| | (primer) | amplification | |
| LL078 | oligos | LL028 | atcagtgattggtgggttaatg |
| | (primer) | amplification | |
| LL079 | oligos | LL027 | ggagaccactccagattcca |
| | (primer) | amplification- | |
| | | phospho | |
| LL080 | oligos | LL027 | cagtaaggggcaaacagtctga |
| | (primer) | amplification- | |
| | | phospho | |
| LL081 | oligos | LL027 | gaacgttcactgaaatcatgg |
| | (primer) | amplification- | |
| | | phospho | |
| LL082 | oligos | LL027 | atcagtgattggtgggttaatg |
| | (primer) | amplification- | |
| | | phospho | |
| LL083 | oligos | | agtcacccaagtgtggtcta |
| | (primer) | | |
| LL084 | oligos | | ccatagagcccaccgcatcc |
| | (primer) | | |
| LL085 | oligos | phospho | agtcacccaagtgtggtcta |
| | (primer) | | |
| LL086 | oligos | phospho | ccatagagcccaccgcatcc |
| | (primer) | | |
| LL087 | oligos | LL027 | GGAGACCACTCCAGATTCCA |
| | (primer) | amplification | |
| LL088 | oligos | LL028 | TGGAGAAGGGGCTTAGAATGAG |
| | (primer) | amplification | |
| LL089 | oligos | LL027 | GGAGACCACTCCAGATTCCA |
| | (primer) | amplification- | |
| | | phospho | |
| LL090 | oligos | LL028 | TGGAGAAGGGGCTTAGAATGAG |
| | (primer) | amplification- | |
| | | phospho | |
| LL091 | oligos | | gtatatgcacacacacactg |
| | (primer) | | |
| LL092 | oligos | | ctgctgcaggataccatgag |
| | (primer) | | |
| LL093 | oligos | phospho | gtatatgcacacacacactg |
| | (primer) | | |
| LL094 | oligos | phospho | ctgctgcaggataccatgag |
| | (primer) | | |
| LL095 | oligos | | gtttggctccagggtaatcg |
| | (primer) | | |
| LL096 | oligos | | tggtaggtaaagaggagagatga |
| | (primer) | | |
| LL097 | oligos | phospho | gtttggctccagggtaatcg |
| | (primer) | | |
| LL098 | oligos | phospho | tggtaggtaaagaggagagatga |
| | (primer) | | |
| LL099 | oligos | | ttataccactaatgagagtttcctacc |
| | (primer) | | |
| LL100 | oligos | | tgaaccttgatcatcccacct |
| | (primer) | | |
| LL101 | oligos | | agtcacccaagtgtggtcta |
| | (primer) | | |
| LL102 | oligos | | ccatagagcccaccgcatcc |
| | (primer) | | |
| LL103 | oligos | phospho | agtcacccaagtgtggtcta |
| | (primer) | | |
| LL104 | oligos | phospho | ccatagagcccaccgcatcc |
| | (primer) | | |
| LL105 | oligos | | gggcagagcgcacatcgccc |
| | (primer) | | |
| LL106 | oligos | phospho | gggcagagcgcacatcgccc |
| | (primer) | | |
| LL107 | oligos | | cccacgagacaaatatatac |
| | (primer) | | |
| LL108 | oligos | | tgtgaaccttgatcatccca |
| | (primer) | | |
| LL109 | oligos | phospho | cccacgagacaaatatatac |
| | (primer) | | |
| LL110 | oligos | phospho | tgtgaaccttgatcatccca |
| | (primer) | | |
| LL111 | oligos | | agtaaaaacagccaagacaa |
| | (primer) | | |
| LL112 | oligos | | ggcctcggcgctgacgatct |
| | (primer) | | |
| LL113 | oligos | phospho | agtaaaaacagccaagacaa |
| | (primer) | | |
| LL114 | oligos | phospho | ggcctcggcgctgacgatct |
| | (primer) | | |
| LL115 | oligos | | ggctccggcgagggcagggg |
| | (primer) | | |
| LL116 | oligos | | tccattttccctggtagctg |
| | (primer) | | |
| LL117 | oligos | phospho | ggctccggcgagggcagggg |
| | (primer) | | |
| LL118 | oligos | phospho | tccattttccctggtagctg |
| | (primer) | | |
| LL119 | oligos | ll027 | ggagaccactccagattcca |
| | (primer) | | |
| LL120 | oligos | ll027 | cagtaaggggcaaacagtctga |
| | (primer) | | |
| LL121 | oligos | ll027 | ggagaccactccagattcca |
| | (primer) | | |
| LL122 | oligos | ll027 | cagtaaggggcaaacagtctgag |
| | (primer) | | |
| LL123 | oligos | ll027 | ggagaccactccagattccaa |
| | (primer) | | |
| LL124 | oligos | ll027 | cagtaaggggcaaacagtctga |
| | (primer) | | |
| LL125 | oligos | ll028 | ttcactgaaatcatggcctct |
| | (primer) | | |
| LL126 | oligos | ll028 | atcagtgattggtgggttaatg |
| | (primer) | | |
| LL127 | oligos | ll028 | gttcactgaaatcatggcctct |
| | (primer) | | |
| LL128 | oligos | ll028 | tcagtgattggtgggttaatga |
| | (primer) | | |
| LL129 | oligos | ll028 | ttcactgaaatcatggcctct |
| | (primer) | | |
| LL130 | oligos | ll028 | atcagtgattggtgggttaatga |
| | (primer) | | |
| LL131 | oligos | ll029f | ggagaccactccagattcca |
| | (primer) | | |
| LL132 | oligos | ll029r1 | tggagaaggggcttagaatg |
| | (primer) | | |
| LL133 | oligos | ll029r2 | tggagaaggggcttagaatga |
| | (primer) | | |
| LL134 | oligos | ll029r3 | tggagaaggggcttagaatgag |
| | (primer) | | |
| LL135 | oligos | ll050f | taaaaacagccaagacaatcagg |
| | (primer) | | |
| LL136 | oligos | ll050r1 | cgctgacgatctgggtgac |
| | (primer) | | |
| LL137 | oligos | ll050r2 | cgctgacgatctgggtga |
| | (primer) | | |
| LL138 | oligos | ll050f3 | agtaaaaacagccaagacaatca |
| | (primer) | | |
| LL139 | oligos | ll050r3 | ctgacgatctgggtgacg |
| | (primer) | | |
| LL140 | oligos | ll051f1 | ccaagcaaggctttgaaaaa |
| | (primer) | | |
| LL141 | oligos | ll051r | tccattttccctggtagctg |
| | (primer) | | |
| LL142 | oligos | ll051f2 | caccaagcaaggctttgaa |
| | (primer) | | |
| LL143 | oligos | ll051f3 | ccaagcaaggctttgaaaaat |
| | (primer) | | |
| LL144 | oligos | ll052 | acagctggctccagggaag |
| | (primer) | | |
| LL145 | oligos | ll052 | ggcattcaggtgtgaagtga |
| | (primer) | | |
| LL146 | oligos | ll052 | agggcattcaggtgtgaagt |
| | (primer) | | |
| LL147 | oligos | ll052 | cagctggctccagggaag |
| | (primer) | | |
| LL148 | oligos | ll052 | ggcattcaggtgtgaagtga |
| | (primer) | | |
| LL149 | oligos | ll072 | AGTCACCCAAGTGTGGTCTAATATAAATC |
| | (primer) | | |
| LL150 | oligos | ll072 | ccatagagcccaccgcatcc |
| | (primer) | | |
| LL151 | oligos | puc57 | gtgctgcaaggcgattaagt |
| | (primer) | | |
| LL152 | oligos | | ggctcgtatgttgtgtggaa |
| | (primer) | | |
| LL153 | oligos | ppuc57 | gtgctgcaaggcgattaagt |
| | (primer) | | |
| LL154 | oligos | | ggctcgtatgttgtgtggaa |
| | (primer) | | |
| LL155 | oligos | | ctgcaaggcgattaagttgg |
| | (primer) | | |
| LL156 | oligos | puc57B | ggctcgtatgttgtgtggaa |
| | (primer) | | |
| LL157 | oligos | TRBC1 F PCR | TCCTACCTCGAGTTTCAGGAT |
| | (primer) | primer | |
| LL158 | oligos | TRBC1 R PCR | ATTCTCCTTCATGGTGTGCG |
| | (primer) | primer | |
| LL159 | oligos | TRBC2 F PCR | ATCACCTGGAATGTTAGGCAGTG |
| | (primer) | primer | |
| LL160 | oligos | TRBC2 R PCR | AGCTTAGCTCTAAGGTGTCAGG |
| | (primer) | primer | |
| LL161 | oligos | trbc12 | GCAATGTGCATCCATGGGAC |
| | (primer) | deletion | |
| LL162 | oligos | | GCTGACCCTGTGAACCTTGA |
| | (primer) | | |
| LL163 | oligos | | AGAGTTTCCTACCTCGAGTTTCA |
| | (primer) | | |
| LL164 | oligos | | CTCCTTCATGGTGTGCGCT |
| | (primer) | | |
| LL165 | oligos | | ACCCATAGGGTGGATACAAAAGAC |
| | (primer) | | |
| LL166 | oligos | | ATGGGATGCACACCACTCAGAT |
| | (primer) | | |
| LL167 | oligos | | GGGGAGACAGTAGGCAATGT |
| | (primer) | | |
| LL168 | oligos | | GCTGACCCTGTGAACCTTGAT |
| | (primer) | | |
| LL169 | oligos | phospho | ttataccactaatgagagtttcctacc |
| | (primer) | | |
| LL170 | oligos | phospho | tgaaccttgatcatcccacct |
| | (primer) | | |
| LL171 | oligos | | cccacgagacaaatatatac |
| | (primer) | | |
| LL172 | oligos | | tgtgaaccttgatcatccca |
| | (primer) | | |
| LL173 | oligos | phospho | cccacgagacaaatatatac |
| | (primer) | | |
| LL174 | oligos | phospho | tgtgaaccttgatcatccca |
| | (primer) | | |
| LL175 | oligos | Tye665 label | taatagtaatcaattacggggtca |
| | (primer) | for pDonor | |
| | | plasmid PCR-F | |
| LL176 | oligos | Tye665 label | gatacattgatgagtttggacaaa |
| | (primer) | for pDonor | |
| | | plasmid PCR-R | |
| LL177 | oligos | LL027 HindIII | aagcttatgggagaccactccagattccaa |
| | (primer) | F | |
| LL178 | oligos | LL027 NotI R | cagactgtttgccccttactgtaagcggccgc |
| | (primer) | | |
| LL179 | oligos | IDT HPRT | |
| | (primer) | human PCR | |
| | | primer mix | |
| LL180 | oligos | Synthego | |
| | (primer) | cdc42 human | |
| | | PCR mix | |
| LL181 | oligos | Synthego | |
| | (primer) | cdc42 human | |
| | | seq primer | |
| LL182- | sgRNA | BCL11A | gcttgtcaaggctattggtcaGTTTTAGAGCTAGAAATAGCAAGTTAAAATAAGGCTAG |
| nuc5 | | (nuc5) | TCCGTTATCAACTTGAAAAAGTGGCACCGAGTCGGTGCTTTTTTT |
| LL183 | | | |
| LL184 | | | |
| LL185 | | | |
| LL186 | | | |
| LL187 | | | |
| LL188 | | | |
| LL189 | | | |
| LL190 | | | |
| LL191 | | | |
| LL192 | | | |
| LL193 | | | |
| LL194 | | | |
| LL195 | | | |
| LL196 | | | |
| LL197 | | | |
| LL198 | | | |
| LL199 | | | |
| LL200 | | | |
| LL201 | oligos | trac left | cataccataaacctcccattc |
| | (primer) | arm-f | |
| LL202 | oligos | gfp-r | ccatctaattcaacaagaat |
| | (primer) | | |
| LL203 | oligos | gfp-f | gaccgccgccgggatcactc |
| | (primer) | | |
| LL204 | oligos | trac right | ggagaggcaacttggagaag |
| | (primer) | arm-r | |
| LL205 | oligos | sfGFP-N | gagctgttcaccggggtggt |
| | (primer) | | |
| LL206 | oligos | sfGFP-c | gctcgtccatgccgtgagtg |
| | (primer) | | |
| LL207 | oligos | trbc1 left | ggactcagatgtaatggaaa |
| | (primer) | arm-f | |
| LL208 | oligos | RFP-r | cttgaagcgcatgaactcggt |
| | (primer) | | |
| LL209 | oligos | rfp-f | cgagcgcaccgagggccgcc |
| | (primer) | | |
| LL210 | oligos | trbc2 right | ccattcagcctctatgcttc |
| | (primer) | arm-r | |
| LL211 | oligos | 2a-f | gcaggggaagtctactaaca |
| | (primer) | | |
| LL212 | oligos | rfp-C | caggaacaggtggtggcggc |
| | (primer) | | |
| LL213 | oligos | gfp-f2 | gctgctgggattacacatg |
| | (primer) | | |
| LL214 | oligos | 2A-f2 | catgcggggacgtggaggaa |
| | (primer) | | |
| LL215 | oligos | sfGFP-c2 | ctcatccatgccatgtgtaa |
| | (primer) | | |
| LL216 | oligos | TRAP primer | TTCGATGTTCACCATAATCACTTGG |
| | (primer) | | |
| LL217 | oligos | TRAP primer | CCACATCTGCTGCAGGATACC |
| | (primer) | | |
| LL218 | oligos | TRAP primer | ACATGTTCGATGTTCACCATAATCA |
| | (primer) | | |
| LL219 | oligos | TRAP primer | TGAGACTCACCTCCCATCTTCA |
| | (primer) | | |
| LL220 | oligos | TRBP primer | CACTGAGATGAGGGAGGGGA |
| | (primer) | | |
| LL221 | oligos | TRBP primer | GACCACACCACAGTGGAGAC |
| | (primer) | | |
| LL222 | oligos | TRBP primer | TAGGTGAGGTGTCCTCCTGG |
| | (primer) | | |
| LL223 | oligos | TRBP primer | TGCTCTGTGTCCTTGTCGTC |
| | (primer) | | |
| LL224 | sgRNA | Marson paper | AGAGTCTCTCAGCTGGTACAGTTTTAGAGCTAGAAATAGCAAGTTAAAATAA |
| | | TRAC exon1 | GGCTAGTCCGTTATCAACTTGAAAAAGTGGCACCGAGTCGGTGCTTTTTTT |
| | | guide | |
| LL225 | sgRNA | Marson paper | CAAACACAGCGACCTTGGGTGTTTTAGAGCTAGAAATAGCAAGTTAAAATAA |
| | | TRBC1 exon1 | GGCTAGTCCGTTATCAACTTGAAAAAGTGGCACCGAGTCGGTGCTTTTTTT |
| | | guide | |
| LL226 | plasmid | Marson paper | TTTCAGGTTTCCTTGAGTGGCAGGCCAGGCCTGGCCGTGAACGTTCACTGA |
| | | TRAC donor- | AATCATGGCCTCTTGGCCAAGATTGATAGCTTGTGCCTGTCCCTGAGTCCC |
| | | duplicate of | AGTCCATCACGAGCAGCTGGTTTCTAAGATGCTATTTCCCGTATAAAGCATG |
| | | LL230 | AGACCGTGACTTGCCAGCCCCACAGAGCCCCGCCCTTGTCCATCACTGGC |
| | | | ATCTGGACTCCAGCCTGGGTTGGGGCAAAGAGGGAAATGAGATCATGTCC |
| | | | TAACCCTGATCCTCTTGTCCCACAGATATCCAGAACCCTGACCCTGCCTCC |
| | | | GGATCCGGAGAGGGCAGGGGATCTCTCCTTACTTGTGGCGACGTGGAGGA |
| | | | GAACCCCGGCCCCATGAGCATCGGCCTCCTGTGCTGTGCAGCCTTGTCTC |
| | | | TCCTGTGGGCAGGTCCAGTGAATGCTGGTGTCACTCAGACCCCAAAATTCC |
| | | | AGGTCCTGAAGACAGGACAGAGCATGACACTGCAGTGTGCCCAGGATATG |
| | | | AACCATGAATACATGTCCTGGTATCGACAAGACCCAGGCATGGGGCTGAG |
| | | | GCTGATTCATTACTCAGTTGGTGCTGGTATCACTGACCAAGGAGAAGTCCC |
| | | | CAATGGCTACAATGTCTCCAGATCAACCACAGAGGATTTCCCGCTCAGGCT |
| | | | GCTGTCGGCTGCTCCCTCCCAGACATCTGTGTACTTCTGTGCCAGCAGTTA |
| | | | CGTCGGGAACACCGGGGAGCTGTTTTTTGGAGAAGGCTCTAGGCTGACCG |
| | | | TACTGGAGGACCTGAAAAACGTGTTCCCACCCGAGGTCGCTGTGTTTGAGC |
| | | | CATCAGAAGCAGAGATCTCCCACACCCAAAAGGCCACACTGGTATGCCTG |
| | | | GCCACAGGCTTCTACCCCGACCACGTGGAGCTGAGCTGGTGGGTGAATGG |
| | | | GAAGGAGGTGCACAGTGGGGTCAGCACAGACCCGCAGCCCCTCAAGGAG |
| | | | CAGCCCGCCCTCAATGACTCCAGATACTGCCTGAGCAGCCGCCTGAGGGT |
| | | | CTCGGCCACCTTCTGGCAGAACCCCCGCAACCACTTCCGCTGTCAAGTCC |
| | | | AGTTCTACGGGCTCTCGGAGAATGACGAGTGGACCCAGGATAGGGCCAAA |
| | | | CCCGTCACCCAGATCGTCAGCGCCGAGGCCTGGGGTAGAGCAGACTGTG |
| | | | GCTTCACCTCCGAGTCTTACCAGCAAGGGGTCCTGTCTGCCACCATCCTCT |
| | | | ATGAGATCTTGCTAGGGAAGGCCACCTTGTATGCCGTGCTGGTCAGTGCC |
| | | | CTCGTGCTGATGGCTATGGTCAAGAGAAAGGATTCCAGAGGCCGGGCCAA |
| | | | GCGGTCCGGATCCGGAGCCACCAACTTCAGCCTGCTGAAGCAGGCCGGC |
| | | | GACGTGGAGGAGAACCCCGGCCCCATGGAGACCCTCTTGGGCCTGCTTAT |
| | | | CCTTTGGCTGCAGCTGCAATGGGTGAGCAGCAAACAGGAGGTGACGCAGA |
| | | | TTCCTGCAGCTCTGAGTGTCCCAGAAGGAGAAAACTTGGTTCTCAACTGCA |
| | | | GTTTCACTGATAGCGCTATTTACAACCTCCAGTGGTTTAGGCAGGACCCTG |
| | | | GGAAAGGTCTCACATCTCTGTTGCTTATTCAGTCAAGTCAGAGAGAGCAAA |
| | | | CAAGTGGAAGACTTAATGCCTCGCTGGATAAATCATCAGGACGTAGTACTTT |
| | | | ATACATTGCAGCTTCTCAGCCTGGTGACTCAGCCACCTACCTCTGTGCTGT |
| | | | GAGGCCCCTGTACGGAGGAAGCTACATACCTACATTTGGAAGAGGAACCA |
| | | | GCCTTATTGTTCATCCGTATATCCAGAACCCTGACCCTGCGGTGTACCAGC |
| | | | TGAGAGACTCTAAATCCAGTGACAAGTCTGTCTGCCTATTCACCGATTTTGA |
| | | | TTCTCAAACAAATGTGTCACAAAGTAAGGATTCTGATGTGTATATCACAGAC |
| | | | AAAACTGTGCTAGACATGAGGTCTATGGACTTCAAGAGCAACAGTGCTGTG |
| | | | GCCTGGAGCAACAAATCTGACTTTGCATGTGCAAACGCCTTCAACAACAGC |
| | | | ATTATTCCAGAAGACACCTTCTTCCCCAGCCCAGGTAAGGGCAGCTTTGGT |
| | | | GCCTTCGCAGGCTGTTTCCTTGCTTCAGGAATGGCCA |
| LL227 | plasmid | | GAATTCTAATGAGAGTTTCCTACCTCGAGTTTCAGGATTACATAGCCATGCA |
| | | | CCAAGCAAGGCTTTGAAAAATAAAGATACACAGATAAATTATTTGGATAGAT |
| | | | GATCAGACAAGCCTCAGTAAAAACAGCCAAGACAATCAGGATATAATGTGA |
| | | | CCATAGGAAGCTGGGGAGACAGTAGGCAATGTGCATCCATGGGACAGCAT |
| | | | AGAAAGGAGGGGCAAAGTGGAGAGAGAGCAACAGACACTGGGATGGTGA |
| | | | CCCCAAAACAATGAGGGCCTAGAATGACATAGTTGTGCTTCATTACGGCCC |
| | | | ATTCCCAGGGCTCTCTCTCACACACACAGAGCCCCTACCAGAACCAGACAG |
| | | | CTCTCAGAGCAACCCTGGCTCCAACCCCTCTTCCCTTTCCAGAGTCCGGAT |
| | | | CCGGAGAGGGCAGGGGATCTCTCCTTACTTGTGGCGACGTGGAGGAGAAC |
| | | | CCCGGCCCCATGGAGACCCTCTTGGGCCTGCTTATCCTTTGGCTGCAGCT |
| | | | GCAATGGGTGAGCAGCAAACAGGAGGTGACGCAGATTCCTGCAGCTCTGA |
| | | | GTGTCCCAGAAGGAGAAAACTTGGTTCTCAACTGCAGTTTCACTGATAGCG |
| | | | CTATTTACAACCTCCAGTGGTTTAGGCAGGACCCTGGGAAAGGTCTCACAT |
| | | | CTCTGTTGCTTATTCAGTCAAGTCAGAGAGAGCAAACAAGTGGAAGACTTAA |
| | | | TGCCTCGCTGGATAAATCATCAGGACGTAGTACTTTATACATTGCAGCTTCT |
| | | | CAGCCTGGTGACTCAGCCACCTACCTCTGTGCTGTGAGGCCCCTGTACGG |
| | | | AGGAAGCTACATACCTACATTTGGAAGAGGAACCAGCCTTATTGTTCATCC |
| | | | GTATATCCAGAACCCTGACCCTGCCGTGTACCAGCTGAGAGACTCTAAATC |
| | | | CAGTGACAAGTCTGTCTGCCTATTCACCGATTTTGATTCTCAAACAAATGTG |
| | | | TCACAAAGTAAGGATTCTGATGTGTATATCACAGACAAAACTGTGCTAGACA |
| | | | TGAGGTCTATGGACTTCAAGAGCAACAGTGCTGTGGCCTGGAGCAACAAAT |
| | | | CTGACTTTGCATGTGCAAACGCCTTCAACAACAGCATTATTCCAGAAGACAC |
| | | | CTTCTTCCCCAGCCCAGAAAGTTCCTGTGATGTCAAGCTGGTCGAGAAAAG |
| | | | CTTTGAAACAGATACGAACCTAAACTTTCAAAACCTGTCAGTGATTGGGTTC |
| | | | CGAATCCTCCTCCTGAAAGTGGCCGGGTTTAATCTGCTCATGACGCTGCGG |
| | | | CTGTGGTCCAGCCGGGCCAAGCGGTCCGGATCCGGAGCCACCAACTTCA |
| | | | GCCTGCTGAAGCAGGCCGGCGACGTGGAGGAGAACCCCGGCCCCATGAG |
| | | | CATCGGCCTCCTGTGCTGTGCAGCCTTGTCTCTCCTGTGGGCAGGTCCAG |
| | | | TGAATGCTGGTGTCACTCAGACCCCAAAATTCCAGGTCCTGAAGACAGGAC |
| | | | AGAGCATGACACTGCAGTGTGCCCAGGATATGAACCATGAATACATGTCCT |
| | | | GGTATCGACAAGACCCAGGCATGGGGCTGAGGCTGATTCATTACTCAGTTG |
| | | | GTGCTGGTATCACTGACCAAGGAGAAGTCCCCAATGGCTACAATGTCTCCA |
| | | | GATCAACCACAGAGGATTTCCCGCTCAGGCTGCTGTCGGCTGCTCCCTCC |
| | | | CAGACATCTGTGTACTTCTGTGCCAGCAGTTACGTCGGGAACACCGGGGA |
| | | | GCTGTTTTTTGGAGAAGGCTCTAGGCTGACCGTACTGGAGGACCTGAACAA |
| | | | AGTGTTCCCACCCGAGGTCGCTGTGTTTGAGCCATCAGAAGCAGAGATCTC |
| | | | CCACACCCAAAAGGCCACACTGGTGTGCCTGGCCACAGGCTTCTTCCCTG |
| | | | ACCACGTGGAGCTGAGCTGGTGGGTGAATGGGAAGGAGGTGCACAGTGG |
| | | | GGTCAGCACGGACCCGCAGCCCCTCAAGGAGCAGCCCGCCCTCAATGACT |
| | | | CCAGATACTGCCTGAGCAGCCGCCTGAGGGTCTCGGCCACCTTCTGGCAG |
| | | | AACCCCCGCAACCACTTCCGCTGTCAAGTCCAGTTCTACGGGCTCTCGGA |
| | | | GAATGACGAGTGGACCCAGGATAGGGCCAAACCCGTCACCCAGATCGTCA |
| | | | GCGCCGAGGCCTGGGGTAGAGCAGGATATC |
| LL228 | dsDNA | | AAGCTTTTTCAGGTTTCCTTGAGTGGCAGGCCAGGCCTGGCCGTGAACGTT |
| | | | CACTGAAATCATGGCCTCTTGGCCAAGATTGATAGCTTGTGCCTGTCCCTG |
| | | | AGTCCCAGTCCATCACGAGCAGCTGGTTTCTAAGATGCTATTTCCCGTATAA |
| | | | AGCATGAGACCGTGACTTGCCAGCCCCACAGAGCCCCGCCCTTGTCCATC |
| | | | ACTGGCATCTGGACTCCAGCCTGGGTTGGGGCAAAGAGGGAAATGAGATC |
| | | | ATGTCCTAACCCTGATCCTCTTGTCCCACAGATATCCAGAACCCTGACCCT |
| | | | GCCTCCGGATCCGGAGCCACCAACTTCAGCCTGCTGAAGCAGGCCGGCG |
| | | | ACGTGGAGGAGAACCCCGGCCCCATGGAGACCCTCTTGGGCCTGCTTATC |
| | | | CTTTGGCTGCAGCTGCAATGGGTGAGCAGCAAACAGGAGGTGACGCAGAT |
| | | | TCCTGCAGCTCTGAGTGTCCCAGAAGGAGAAAACTTGGTTCTCAACTGCAG |
| | | | TTTCACTGATAGCGCTATTTACAACCTCCAGTGGTTTAGGCAGGACCCTGG |
| | | | GAAAGGTCTCACATCTCTGTTGCTTATTCAGTCAAGTCAGAGAGAGCAAACA |
| | | | AGTGGAAGACTTAATGCCTCGCTGGATAAATCATCAGGACGTAGTACTTTAT |
| | | | ACATTGCAGCTTCTCAGCCTGGTGACTCAGCCACCTACCTCTGTGCTGTGA |
| | | | GGCCCCTGTACGGAGGAAGCTACATACCTACATTTGGAAGAGGAACCAGC |
| | | | CTTATTGTTCATCCGTATATCCAGAACCCTGACCCTGCGGTGTACCAGCTG |
| | | | AGAGACTCTAAATCCAGTGACAAGTCTGTCTGCCTATTCACCGATTTTGATT |
| | | | CTCAAACAAATGTGTCACAAAGTAAGGATTCTGATGTGTATATCACAGACAA |
| | | | AACTGTGCTAGACATGAGGTCTATGGACTTCAAGAGCAACAGTGCTGTGGC |
| | | | CTGGAGCAACAAATCTGACTTTGCATGTGCAAACGCCTTCAACAACAGCATT |
| | | | ATTCCAGAAGACACCTTCTTCCCCAGCCCAGGTAAGGGCAGCTTTGGTGCC |
| | | | TTCGCAGGCTGTTTCCTTGCTTCAGGAATGGCCAGCGGCCGC |
| LL229 | dsDNA | | AAGCTTTAATGAGAGTTTCCTACCTCGAGTTTCAGGATTACATAGCCATGCA |
| | | | CCAAGCAAGGCTTTGAAAAATAAAGATACACAGATAAATTATTTGGATAGAT |
| | | | GATCAGACAAGCCTCAGTAAAAACAGCCAAGACAATCAGGATATAATGTGA |
| | | | CCATAGGAAGCTGGGGAGACAGTAGGCAATGTGCATCCATGGGACAGCAT |
| | | | AGAAAGGAGGGGCAAAGTGGAGAGAGAGCAACAGACACTGGGATGGTGA |
| | | | CCCCAAAACAATGAGGGCCTAGAATGACATAGTTGTGCTTCATTACGGCCC |
| | | | ATTCCCAGGGCTCTCTCTCACACACACAGAGCCCCTACCAGAACCAGACAG |
| | | | CTCTCAGAGCAACCCTGGCTCCAACCCCTCTTCCCTTTCCAGAGTCCGGAT |
| | | | CCGGAGCCACCAACTTCAGCCTGCTGAAGCAGGCCGGCGACGTGGAGGA |
| | | | GAACCCCGGCCCCATGAGCATCGGCCTCCTGTGCTGTGCAGCCTTGTCTC |
| | | | TCCTGTGGGCAGGTCCAGTGAATGCTGGTGTCACTCAGACCCCAAAATTCC |
| | | | AGGTCCTGAAGACAGGACAGAGCATGACACTGCAGTGTGCCCAGGATATG |
| | | | AACCATGAATACATGTCCTGGTATCGACAAGACCCAGGCATGGGGCTGAG |
| | | | GCTGATTCATTACTCAGTTGGTGCTGGTATCACTGACCAAGGAGAAGTCCC |
| | | | CAATGGCTACAATGTCTCCAGATCAACCACAGAGGATTTCCCGCTCAGGCT |
| | | | GCTGTCGGCTGCTCCCTCCCAGACATCTGTGTACTTCTGTGCCAGCAGTTA |
| | | | CGTCGGGAACACCGGGGAGCTGTTTTTTGGAGAAGGCTCTAGGCTGACCG |
| | | | TACTGGAGGACCTGAACAAAGTGTTCCCACCCGAGGTCGCTGTGTTTGAGC |
| | | | CATCAGAAGCAGAGATCTCCCACACCCAAAAGGCCACACTGGTGTGCCTG |
| | | | GCCACAGGCTTCTTCCCTGACCACGTGGAGCTGAGCTGGTGGGTGAATGG |
| | | | GAAGGAGGTGCACAGTGGGGTCAGCACGGACCCGCAGCCCCTCAAGGAG |
| | | | CAGCCCGCCCTCAATGACTCCAGATACTGCCTGAGCAGCCGCCTGAGGGT |
| | | | CTCGGCCACCTTCTGGCAGAACCCCCGCAACCACTTCCGCTGTCAAGTCC |
| | | | AGTTCTACGGGCTCTCGGAGAATGACGAGTGGACCCAGGATAGGGCCAAA |
| | | | CCCGTCACCCAGATCGTCAGCGCCGAGGCCTGGGGTAGAGCAGGCGGCCGC |
| LL230 | ssDNA | TRAC NYESO | TTTCAGGTTTCCTTGAGTGGCAGGCCAGGCCTGGCCGTGAACGTTCACTGA |
| | | HDR donor for | AATCATGGCCTCTTGGCCAAGATTGATAGCTTGTGCCTGTCCCTGAGTCCC |
| | | Cas9 sgLL224 | AGTCCATCACGAGCAGCTGGTTTCTAAGATGCTATTTCCCGTATAAAGCATG |
| | | | AGACCGTGACTTGCCAGCCCCACAGAGCCCCGCCCTTGTCCATCACTGGC |
| | | | ATCTGGACTCCAGCCTGGGTTGGGGCAAAGAGGGAAATGAGATCATGTCC |
| | | | TAACCCTGATCCTCTTGTCCCACAGATATCCAGAACCCTGACCCTGCCTCC |
| | | | GGATCCGGAGAGGGCAGGGGATCTCTCCTTACTTGTGGCGACGTGGAGGA |
| | | | GAACCCCGGCCCCATGAGCATCGGCCTCCTGTGCTGTGCAGCCTTGTCTC |
| | | | TCCTGTGGGCAGGTCCAGTGAATGCTGGTGTCACTCAGACCCCAAAATTCC |
| | | | AGGTCCTGAAGACAGGACAGAGCATGACACTGCAGTGTGCCCAGGATATG |
| | | | AACCATGAATACATGTCCTGGTATCGACAAGACCCAGGCATGGGGCTGAG |
| | | | GCTGATTCATTACTCAGTTGGTGCTGGTATCACTGACCAAGGAGAAGTCCC |
| | | | CAATGGCTACAATGTCTCCAGATCAACCACAGAGGATTTCCCGCTCAGGCT |
| | | | GCTGTCGGCTGCTCCCTCCCAGACATCTGTGTACTTCTGTGCCAGCAGTTA |
| | | | CGTCGGGAACACCGGGGAGCTGTTTTTTGGAGAAGGCTCTAGGCTGACCG |
| | | | TACTGGAGGACCTGAAAAACGTGTTCCCACCCGAGGTCGCTGTGTTTGAGC |
| | | | CATCAGAAGCAGAGATCTCCCACACCCAAAAGGCCACACTGGTATGCCTG |
| | | | GCCACAGGCTTCTACCCCGACCACGTGGAGCTGAGCTGGTGGGTGAATGG |
| | | | GAAGGAGGTGCACAGTGGGGTCAGCACAGACCCGCAGCCCCTCAAGGAG |
| | | | CAGCCCGCCCTCAATGACTCCAGATACTGCCTGAGCAGCCGCCTGAGGGT |
| | | | CTCGGCCACCTTCTGGCAGAACCCCCGCAACCACTTCCGCTGTCAAGTCC |
| | | | AGTTCTACGGGCTCTCGGAGAATGACGAGTGGACCCAGGATAGGGCCAAA |
| | | | CCCGTCACCCAGATCGTCAGCGCCGAGGCCTGGGGTAGAGCAGACTGTG |
| | | | GCTTCACCTCCGAGTCTTACCAGCAAGGGGTCCTGTCTGCCACCATCCTCT |
| | | | ATGAGATCTTGCTAGGGAAGGCCACCTTGTATGCCGTGCTGGTCAGTGCC |
| | | | CTCGTGCTGATGGCTATGGTCAAGAGAAAGGATTCCAGAGGCCGGGCCAA |
| | | | GCGGTCCGGATCCGGAGCCACCAACTTCAGCCTGCTGAAGCAGGCCGGC |
| | | | GACGTGGAGGAGAACCCCGGCCCCATGGAGACCCTCTTGGGCCTGCTTAT |
| | | | CCTTTGGCTGCAGCTGCAATGGGTGAGCAGCAAACAGGAGGTGACGCAGA |
| | | | TTCCTGCAGCTCTGAGTGTCCCAGAAGGAGAAAACTTGGTTCTCAACTGCA |
| | | | GTTTCACTGATAGCGCTATTTACAACCTCCAGTGGTTTAGGCAGGACCCTG |
| | | | GGAAAGGTCTCACATCTCTGTTGCTTATTCAGTCAAGTCAGAGAGAGCAAA |
| | | | CAAGTGGAAGACTTAATGCCTCGCTGGATAAATCATCAGGACGTAGTACTTT |
| | | | ATACATTGCAGCTTCTCAGCCTGGTGACTCAGCCACCTACCTCTGTGCTGT |
| | | | GAGGCCCCTGTACGGAGGAAGCTACATACCTACATTTGGAAGAGGAACCA |
| | | | GCCTTATTGTTCATCCGTATATCCAGAACCCTGACCCTGCGGTGTACCAGC |
| | | | TGAGAGACTCTAAATCCAGTGACAAGTCTGTCTGCCTATTCACCGATTTTGA |
| | | | TTCTCAAACAAATGTGTCACAAAGTAAGGATTCTGATGTGTATATCACAGAC |
| | | | AAAACTGTGCTAGACATGAGGTCTATGGACTTCAAGAGCAACAGTGCTGTG |
| | | | GCCTGGAGCAACAAATCTGACTTTGCATGTGCAAACGCCTTCAACAACAGC |
| | | | ATTATTCCAGAAGACACCTTCTTCCCCAGCCCAGGTAAGGGCAGCTTTGGT |
| | | | GCCTTCGCAGGCTGTTTCCTTGCTTCAGGAATGGCCA |
| LL231 | oligos | adaptor-f | GAAGTGCCATTCCGCCTGAC |
| | (primer) | | |
| LL232 | oligos | adaptor-r | CACTGAGCCTCCACCTAGCC |
| | (primer) | | |
| LL233 | oligos | RELA primer | TTCTAGGGAGCAGGTCCTGACT |
| | (primer) | F - For PCR | |
| LL234 | oligos | RELA primer | TCCTTTCCTACAAGCTCGTGGG |
| | (primer) | R - For PCR | |
| LL235 | oligos | RELA primer- | AGTACAGAGGCCCAGACATCCAA |
| | (primer) | for sequencing | |
| LL236 | sgRNA | GFP sgRNA | GAGCTGGACGGCGACGTAAA |
| | | | GTTTTAGAGCTAGAAATAGCAAGTTAAAATAAGGCTAGTCCGTTATCAACTTG |
| | | | AAAAAGTGGCACCGAGTCGGTGCTTTTTTT |
| LL237 | oligos | TRBC1 primer F | TGGGGAGACAGTAGGCAATGTG |
| | (primer) | | |
| LL238 | oligos | TRBC1&2 | AGCCCGTAGAACTGGACTTGAC |
| | (primer) | primer R | |
| LL239 | oligos | TRBC2 primer F | GGCAAGGAAGGGGTAGAACCAT |
| | (primer) | | |
| LL240 | oligos | TRAC primer F | ggttggggcaaagagggaaatg |
| | (primer) | | |
| LL241 | oligos | TRAC primer R | ggcctggagcaacaaatctgac |
| | (primer) | | |
| LL242 | oligos | TRBP primer F | AGTCTAAACCCACCTCTTGAGG |
| | (primer) | | |
| LL243 | oligos | L230 primer F | TTTCAGGTTTCCTTGAGTGGCA |
| | (primer) | | |
| LL244 | oligos | L230 primer R | TGGCCATTCCTGAAGCAAGGA |
| | (primer) | | |
| LL245 | oligos | L230 primer F | TTTCAGGTTTCCTTGAGTGG |
| | (primer) | | |
| LL246 | oligos | L230 primer R | TGGCCATTCCTGAAGCAAGG |
| | (primer) | | |
| LL247 | oligos | l027 donor | ACCTCCCATTCTGCTAATGCC |
| | (primer) | genotyping | |
| | | primer full 1 | |
| LL248 | oligos | l027 donor | TGGGTTAATGAGTGACTGCGT |
| | (primer) | genotyping | |
| | | primer full 1 | |
| LL249 | oligos | l027 donor | CCAGCCTAAGTTGGGGAGAC |
| | (primer) | genotyping | |
| | | primer full 2 | |
| LL250 | oligos | l027 donor | GTGACTGCGTGAGACTGACT |
| | (primer) | genotyping | |
| | | primer full 2 | |
| LL251 | oligos | l224 donor | GCCAGAGTTATATTGCTGGGGT |
| | (primer) | genotyping | |
| | | primer full 1 | |
| LL252 | oligos | l224 donor | AGGGTTTTGGTGGCAATGGA |
| | (primer) | genotyping | |
| | | primer full 1 | |
| LL253 | oligos | l224 donor | AGGTTTCCTTGAGTGGCAGG |
| | (primer) | genotyping | |
| | | primer full 2 | |
| LL254 | oligos | l224 donor | GACTGCCAGAACAAGGCTCA |
| | (primer) | genotyping | |
| | | primer full 2 | |
| LL255 | oligos | l027 donor | CTGCTAATGCCCAGCCTAAGT |
| | (primer) | genotyping | |
| | | primer left 1 | |
| LL256 | oligos | l027 donor | CACGTCCCCGCATGTTAGTAG |
| | (primer) | genotyping | |
| | | primer left 1 | |
| LL257 | oligos | l027 donor | TAAGTTGGGGAGACCACTCCAG |
| | (primer) | genotyping | |
| | | primer left 2 | |
| LL258 | oligos | l027 donor | CTCCACGTCCCCGCATGT |
| | (primer) | genotyping | |
| | | primer left 2 | |
| LL259 | oligos | l027 donor | CATGGCATGGATGAGCTCTACAAAT |
| | (primer) | genotyping | |
| | | primer right 1 | |
| LL260 | oligos | l027 donor | GGAGAAGGGGCTTAGAATGAGG |
| | (primer) | genotyping | |
| | | primer right 1 | |
| LL261 | oligos | l027 donor | ACATGGCATGGATGAGCTCTACAAA |
| | (primer) | genotyping | |
| | | primer right 2 | |
| LL262 | oligos | l027 donor | CAACTTGGAGAAGGGGCTTAGA |
| | (primer) | genotyping | |
| | | primer right 2 | |
| LL263 | oligos | l230 donor | GCCAGAGTTATATTGCTGGGGT |
| | (primer) | genotyping | |
| | | primer left 1 | |
| LL264 | oligos | l230 donor | ACGTCGCCACAAGTAAGGAG |
| | (primer) | genotyping | |
| | | primer left 1 | |
| LL265 | oligos | l230 donor | GCTGGGGTTTTGAAGAAGATCCTA |
| | (primer) | genotyping | |
| | | primer left 2 | |
| LL266 | oligos | l230 donor | TCCACGTCGCCACAAGTAA |
| | (primer) | genotyping | |
| | | primer left 2 | |
| LL267 | oligos | l230 donor | CCTGTACGGAGGAAGCTACA |
| | (primer) | genotyping | |
| | | primer right 1 | |
| LL268 | oligos | l230 donor | TGGCAATGGATAAGGCCGAG |
| | (primer) | genotyping | |
| | | primer right 1 | |
| LL269 | oligos | l230 donor | ACCAGCCTTATTGTTCATCCGT |
| | (primer) | genotyping | |
| | | primer right 2 | |
| LL270 | oligos | l230 donor | GATAAGGCCGAGACCACCAA |
| | (primer) | genotyping | |
| | | primer right 2 | |
| LL271 | oligos | l049 donor | GACTCAGATGTAATGGAAAAGTGTC |
| | (primer) | genotyping | |
| | | primer full | |
| LL272 | oligos | l049 donor | AGGAAGAATGAGCTTGAGGTGC |
| | (primer) | genotyping | |
| | | primer full | |
| LL273 | oligos | l049 donor | TATGTGTCACTACCCCACGAGA |
| | (primer) | genotyping | |
| | | primer left | |
| LL274 | oligos | l049 donor | CACGTCCCCGCATGTTAGTAG |
| | (primer) | genotyping | |
| | | primer left | |
| LL275 | oligos | l049 donor | AGCAGTACGAGCGCACC |
| | (primer) | genotyping | |
| | | primer right | |
| LL276 | oligos | l049 donor | GAGCTTGAGGTGCTCCATTCA |
| | (primer) | genotyping | |
| | | primer right | |
| LL277 | ssDNA, | Marson's | TTTCAGGTTTCCTTGAGTGGCAGGCCAGGCCTGGCCGTGAACGTTCACTGA |
| | plasmid | donor LL230 | AATCATGGCCTCTTGGCCAAGATTGATAGCTTGTGCCTGTCCCTGAGTCCCA |
| | | replace | GTCCATCACGAGCAGCTGGTTTCTAAGATGCTATTTCCCGTATAAAGCATGA |
| | | NYSEO with | GACCGTGACTTGCCAGCCCCACAGAGCCCCGCCCTTGTCCATCACTGGCAT |
| | | 2A-sfGFP | CTGGACTCCAGCCTGGGTTGGGGCAAAGAGGGAAATGAGATCATGTCCTAA |
| | | | CCCTGATCCTCTTGTCCCACAGATATCCAGAACCCTGACCCTGCCggctccggc |
| | | | gagggcaggggaagtctactaacatgcggggacgtggaggaaaatcccggcccaagcaaaggagaag |
| | | | aacttttcactggagttgtcccaattcttgttgaattagatggtgatgttaatgggcacaaattttc |
| | | | tgtccgtggagagggtgaaggtgatgctacaaacggaaaactcacccttaaatttatttgcactact |
| | | | ggaaaactacctgttccgtggccaacacttgtcactactctgacctatggtgttcaatgcttttccc |
| | | | gttatccggatcacatgaaacggcatgactttttcaagagtgccatgcccgaaggttatgtacagga |
| | | | acgcactatatctttcaaagatgacgggacctacaagacgcgtgctgaagtcaagtttgaaggtgat |
| | | | acccttgttaatcgtatcgagttaaagggtattgattttaaagaagatggaaacattcttggacaca |
| | | | aactcgagtacaactttaactcacacaatgtatacatcacggcagacaaacaaaagaatggaatcaa |
| | | | agctaacttcaaaattcgccacaacgttgaagatggttccgttcaactagcagaccattatcaacaa |
| | | | aatactccaattggcgatggccctgtccttttaccagacaaccattacctgtcgacacaatctgtcc |
| | | | tttcgaaagatcccaacgaaaagcgtgaccacatggtccttcttgagtttgtaactgctgctgggat |
| | | | tacacatggcatggatgagctctacaaaTAATAGGTGTACCAGCTGAGAGACTCTAAATCCAGTGAC |
| | | | AAGTCTGTCTGCCTATTCACCGATTTTGATTCTCAAACAAATGTGTCACAAAGTAAGGATTCTGATG |
| | | | TGTATATCACAGACAAAACTGTGCTAGACATGAGGTCTATGGACTTCAAGAGC |
| | | | AACAGTGCTGTGGCCTGGAGCAACAAATCTGACTTTGCATGTGCAAACGCC |
| | | | TTCAACAACAGCATTATTCCAGAAGACACCTTCTTCCCCAGCCCAGGTAAGG |
| | | | GCAGCTTTGGTGCCTTCGCAGGCTGTTTCCTTGCTTCAGGAATGGCCA |
| LL278 | ssDNA | 60bp Flag | CAGAACCCTGACCCTGCCgattacaaagacgatgacgataagGTGTACCAGCTGAGAGAC |
| | | oligo for | |
| | | sgLL224 | |
| LL279 | ssDNA | 94bp Flag | CCCTGATCCTCTTGTCCCACAGATATCCAGAACCCTGACCCTGCCgattacaaag |
| | | oligo for | acgatgacgataagGTGTACCAGCTGAGAGACTCTAAATCCAGTGACAAGTCTGTCTGC |
| | | sgLL224 | |
| LL280 | ssDNA | 200bp Flag | CAGCCTGGGTTGGGGCAAAGAGGGAAATGAGATCATGTCCTAACCCTGATC |
| | | oligo for | CTCTTGTCCCACAGATATCCAGAACCCTGACCCTGCCgattacaaagacgatgacgat |
| | | sgLL224 | aagGTGTACCAGCTGAGAGACTCTAAATCCAGTGACAAGTCTGTCTGCCTATT |
| | | | CACCGATTTTGATTCTCAAACAAATGTGTCACAAAGTA |
| LL281 | oligos | GFP primer | ctggtcgagctggacggcgacg |
| | (primer) | for ICE GFP | |
| LL282 | oligos | GFP primer | cacgaactccagcaggaccatg |
| | (primer) | for ICE GFP | |
| LL283 | oligos | Tag reporter F | taatagtaatcaattacggggtca |
| | (primer) | primer | |
| LL284 | oligos | Tag reporter R | gatacattgatgagtttggacaaa |
| | (primer) | primer | |
| LL285 | sgRNA | eGFP Cas9 | cUcgUgaccacccUgaccUaGTTTTAGAGCTAGAAATAGCAAGTTAAAATAAGGCT |
| | | guide for | AGTCCGTTATCAACTTGAAAAAGTGGCACCGAGTCGGTGCTTTTTTT |
| | | 293eGFP | |
| | | GFP -> BFP | |
| | | insertion | |
| LL286 | ssDNA | EGFP2BFP | ACCCTGAAGTTCATCTGCACCACCGGCAAGCTGCCCGTGCCCTGGCCCAC |
| | | donor | CCTCGTGACCACCCTGAGCCACGGGGTGCAGTGCTTCAGCCGCTACCCCG |
| | | | ACCACATGAAGCAGCACGACTTCTTCAAGTCCGCCATGCC |
| LL287 | oligos | EGFP guide | cctctgcctctgagctattc |
| | (primer) | LL236 ICE | |
| | | primer | |
| LL288 | oligos | EGFP guide | atggtgagcaagggcgagg |
| | (primer) | LL236 ICE | |
| | | primer | |
| LL289 | oligos | eGFP | tcgggcatggcggacttgaa |
| | (primer) | sequencing | |
| | | primer | |
| LL290 | ssDNA | phosphorothio | hsACCCTGAAGTTCATCTGCACCACCGGCAAGCTGCCCGTGCCCTGGCCCA |
| | | ate-modified | CCCTCGTGACCACCCTGAGCCACGGGGTGCAGTGCTTCAGCCGCTACCCC |
| | | LL286 at 5′ | GACCACATGAAGCAGCACGACTTCTTCAAGTCCGCCATGCChs |
| | | and 3′ for | |
| | | enhanced KI | |
| LL291 | ssDNA | asymmetric | GCGATGCCACCTACGGCAAGCTGACCCTGAAGTTCATCTGCACCACCGGC |
| | | donor DNA for | AAGCTGCCCGTGCCCTGGCCCACCCTCGTGACCACCCTGAGCCACGGGG |
| | | enhanced KI, | TGCAGTGCTTCAGCCGCTACCCCGACCA |
| | | modify LL286 | |
| LL292 | oligos | eGFP | GTTGCCGTCGTCCTTGAAGAAG |
| | (primer) | sequencing | |
| | | primer | |
| LL293 | oligos | eGFP | TGGCGGATCTTGAAGTTCACCT |
| | (primer) | sequencing | |
| | | primer | |
| LL294 | ssDNA | asymmetric | GTCCCACAGATATCCAGAACCCTGACCCTGCCGTGTCCCCAACTGGGGTAA |
| | | attP donor for | CCTTTGAGTTCTCTCAGTTGGGGGACCAGCTGAGAGACTCTAAATCCAGTGA |
| | | Cas9 sgLL224 | CAAGTCTGTCTGCCTATTCACCGATTTTGATTCTCAAACAAATGTGTCACAAA |
| | | TRAC | GTAAGGATTC |
| LL295 | ssDNA | asymmetric | gcgatgccacctacggcaagctgaccctgaagttcatctgcaccaccggcaagctgcccgtgccctg |
| | | attP donor for | gcccaccctcgtgaccaccctgacCCCCAACTGGGGTAACCTTTGAGTTCTCTCAGTTGGGGG |
| | | Cas9 sgLL285 | ctacggcgtgcagtgcttcagccgctaccccgacca |
| | | EGFP | |
| LL296 | oligos | HPRT1 PCR-F | GAGGCTGAGGCGGGAGAATG |
| | (primer) | for IDT Cpf1 | |
| | | positive | |
| | | control sgRNA | |
| LL297 | oligos | HPRT1 PCR-R | ACATCCATGGGACTTCTGCCTC |
| | (primer) | for IDT Cpf1 | |
| | | positive | |
| | | control sgRNA | |
| LL298 | oligos | HPRT1 seq | AGTCTTTCCTTGGGTGTGTT |
| | (primer) | primer 1 | |
| LL299 | oligos | HPRT1 seq | GCAATTACTTACATTCAAATCCCTG |
| | (primer) | primer 2 | |
| LL300 | ssDNA | LL001 Flag | GTTGcttatcgtcatcgtctttgtaatc |
| | | tetris donor | |
| | | antisense- | |
| | | phospho | |
| | | anneal with | |
| | | LL301 | |
| LL301 | ssDNA | LL001 Flag | CAACgattacaaagacgatgacgataag |
| | | tetris donor | |
| | | sense-phospho | |
| | | anneal with | |
| | | LL300 | |
| LL302 | ssDNA | LL003 Flag | TCTGcttatcgtcatcgtctttgtaatc |
| | | tetris donor | |
| | | antisense- | |
| | | phospho | |
| | | anneal with | |
| | | LL303 | |
| LL303 | ssDNA | LL003 Flag | CAGAgattacaaagacgatgacgataag |
| | | tetris donor | |
| | | sense-phospho | |
| | | anneal with | |
| | | LL302 | |
| LL304 | ssDNA | LL032 Flag | GAATcttatcgtcatcgtctttgtaatc |
| | | tetris donor | |
| | | antisense- | |
| | | phospho | |
| | | anneal with | |
| | | LL305 | |
| LL305 | ssDNA | LL032 Flag | ATTCgattacaaagacgatgacgataag |
| | | tetris donor | |
| | | sense-phospho | |
| | | anneal with | |
| | | LL304 | |
| LL306 | oligos | LL001 T2A | atGGTCTCACAACggctccggcgagggcagggg |
| | (primer) | tetris donor | |
| | | BsaI F-PCR | |
| | | with LL307 and | |
| | | digest with | |
| | | BsaI | |
| LL307 | oligos | LL001 sfGFP | atGGTCTCACAACTTAtttgtagagctcatcca |
| | (primer) | tetris donor | |
| | | BsaI R-PCR | |
| | | with LL306 | |
| | | and digest | |
| | | with BsaI | |
| LL308 | oligos | LL003 T2A | atGGTCTCACAGAggctccggcgagggcagggg |
| | (primer) | tetris donor | |
| | | BsaI F-PCR | |
| | | with LL309 | |
| | | and digest | |
| | | with BsaI | |
| LL309 | oligos | LL003 sfGFP | atGGTCTCACAGATTAtttgtagagctcatcca |
| | (primer) | tetris donor | |
| | | BsaI R-PCR | |
| | | with LL308 | |
| | | and digest | |
| | | with BsaI | |
| LL310 | oligos | LL032 T2A | atGGTCTCAATTCggctccggcgagggcagggg |
| | (primer) | tetris donor | |
| | | BsaI F-PCR | |
| | | with LL311 | |
| | | and digest | |
| | | with BsaI | |
| LL311 | oligos | LL032 sfGFP | atGGTCTCAATTCTTAtttgtagagctcatcca |
| | (primer) | tetris donor | |
| | | BsaI R-PCR | |
| | | with LL310 | |
| | | and digest | |
| | | with BsaI | |
| LL312 | plasmid | CMV-Phi-C31 | |
| | | expression | |
| | | plasmid- | |
| | | Systems | |
| | | Biosciences | |
| LL313 | plasmid | attB donor | |
| | | plasmid PGK- | |
| | | RFP-Systems | |
| | | Biosciences | |
| LL314 | oligos | EGFP indel | ACACTCTTTCCCTACACGACGCTCTTCCGATCTatggtgagcaagggcgagg |
| | (primer) | NGS Forward | |
| | | primer | |
| | | (288 + partial | |
| | | Illumina | |
| | | adapter) | |
| LL315 | oligos | EGFP indel | GACTGGAGTTCAGACGTGTGCTCTTCCGATCTtgtagttgccgtcgtccttg |
| | (primer) | NGS Reverse | |
| | | primer (design | |
| | | new one in | |
| | | order to be | |
| | | less than 500 | |
| | | bp of final | |
| | | producrt) | |
| LL316 | sgRNA | ILR2G guide1 | AACGCTACACGTTTCGTGTT |
| LL317 | sgRNA | ILR2G guide2 | TTCCACAGAGTGGGTTAAAG |
| LL318 | ssDNA | ILR2G donor | ATAAGTTCTCCTTGCCTAGTGTGGATGGGCAGAAACGCTACACGTTTCGTGT |
| | | | TCGGAGCCGCTTTAACCCACTCTGTGGAAGTGCTCAGCATTGGAGTGAATG |
| | | | GAGCCACCCAA |
| LL319 | oligos | TRAC cpf1 | ACACTCTTTCCCTACACGACGCTCTTCCGATCTATCCAGAACCCTGACCCTGC |
| | (primer) | 001, 032 NGS-f | |
| LL320 | oligos | TRAC cpf1 | GACTGGAGTTCAGACGTGTGCTCTTCCGATCTTCTGGACTGCCAGAACAAGG |
| | (primer) | 001, 032 NGS-r | |
| LL321 | oligos | TRBC1 cpf1 | ACACTCTTTCCCTACACGACGCTCTTCCGATCTCCAACCCCTCTTCCCTTTCC |
| | (primer) | 003 NGS-f | |
| LL322 | oligos | TRBC1 cpf1 | GACTGGAGTTCAGACGTGTGCTCTTCCGATCTTTCCCTGGTAGCTGGTCTCA |
| | (primer) | 003 NGS-r | |
| LL323 | oligos | TRBC2 cpf1 | ACACTCTTTCCCTACACGACGCTCTTCCGATCTAGCAAGGAGGACCTAGTAA |
| | (primer) | 003 NGS-f | CATAA |
| LL324 | oligos | TRBC2 cpf1 | GACTGGAGTTCAGACGTGTGCTCTTCCGATCTTTGACAGCGGAAGTGGTTGC |
| | (primer) | 003 NGS-r | |

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

### Aspects

1. A method of generating a targeting ligand that can be used to target cells, tissues, or organs of interest, the method comprising:
(a) identifying one or more cell surface targets for targeting a cell, tissue, or organ of interest;
(b) generating a list of candidate targeting ligands;
(c) producing a library of candidate delivery vehicles, wherein each candidate delivery vehicle displays one or more of the candidate targeting ligands from the list generated in step (b);
(d) contacting the identified one or more cell surface targets of step (a) with the library of candidate delivery vehicles of step (c);
(e) evaluating effectiveness of the candidate targeting ligands to target the one or more cell surface targets based on results of said contacting; and
(f) selecting one or more targeting ligands based on said evaluating.
   2. The method of 1, wherein step (a) comprises calculating a cell, tissue, or organ selectivity index for candidate cell surface targets in order to identify the 3-50 highest expressed surface proteins of the cell, tissue, or organ of interest.
   3. The method of 1, wherein step (a) comprises calculating a cell, tissue, or organ selectivity index for candidate cell surface targets in order to identify the 3-10 highest and uniquely expressed surface proteins of the cell, tissue, or organ of interest.
   4. The method of any one of 1-3, wherein step (b) comprises evaluating crystal structures of the one or more cell surface targets to derive protein-ligand or protein-protein interaction information for the one or more cell surface targets.
   5. The method of 4, wherein the protein-ligand or protein-protein interaction information is used to identify a secondary structure scaffold and the candidate targeting ligands are designed to conform to said secondary structure scaffold.
   6. The method of any one of 1-5, wherein the list of candidate targeting ligands of step (b) includes one or more ligand types selected from the group consisting of: an antibody, a scFv, a nanobody, a chemically synthesized peptide, and a nucleic acid aptamer.
   7. The method of any one of 1-6, wherein the list of candidate targeting ligands of step (b) includes one or more ligands identified by phage display or random peptide library screening.
   8. The method of any one of 1-7, wherein, after step (0, at least one of the selected targeting ligands is subject to mutagenesis to produce a second library of delivery vehicles that display one or more variants of the at least one of the selected targeting ligands, and a second round of contacting, evaluating, and selecting is performed.
   9. The method of any one of 1-7, further comprising, after step (f), generating candidate delivery vehicle formulations for a second round of screening using the one or more selected targeting ligands of step (f).
   10. The method of 9, wherein, after step (f), a machine learning approach is used to approximate an objective function and to generate said candidate delivery vehicle formulations for the second round of screening.
   11. The method of any one of 1-10, wherein:
(i) said contacting of step (d) comprises contacting cells that express said one or more surface targets with the library of candidate delivery vehicles,
(ii) the candidate delivery vehicles of step (c) comprise a detectable payload, and
(iii) said evaluating of step (e) comprises measuring the detectable payload present in said cells after said contacting.
   12. The method of 11, wherein the candidate delivery vehicles of step (c) comprise a targeting ligand fused to the detectable payload.
   13. The method of 11 or 12, wherein said evaluating of step (e) comprises an evaluation of physicochemical data of the candidate delivery vehicles in addition to biological data from said contacting.
   14. The method of 13, wherein said biological data includes one or more of the following parameters: percent of cells that take-up the payload, rate of payload uptake, cell subtype specificity/selectivity, increased cell division activity, gene expression, and cell toxicity.
   15. The method of any one of 1-14, wherein the candidate delivery vehicles of step (c) comprise a targeting ligand fused to an anchoring domain.
   16. The method of 15, wherein the anchoring domain is a charged polymer polypeptide domain that interacts with a detectable payload.
   17. The method of any one of 1-16, wherein the candidate delivery vehicles of step (c) are nanoparticles.
   18. The method of 17, wherein the nanoparticles comprise a core comprising: an anionic polymer composition, a cationic polymer composition, a cationic polypeptide composition, and a detectable payload.
   19. The method of 17, wherein the nanoparticles comprise a core comprising cross-linked polymers.
   20. The method of 17, wherein the nanoparticles comprise a SH residue for coupling to a substrate.
   21. The method of 17, wherein the nanoparticles comprise a solid core particle.
   22. The method of any one of 1-16, wherein the candidate delivery vehicles of step (c) are lipid-based delivery systems that comprise a detectable payload.
   23. The method of any one of 18-22, wherein the detectable payload is a nucleic and/or protein payload.
   24. The method of any one of 17-23, wherein, after step (f), aggregate databases of nanoparticle formulation parameters and their characterized performance metrics are used to predict new candidate formulation performance metrics, whereby these predictions are used to inform and/or guide modifications and refinements to candidate formulations.
   25. The method of any one of 1-24, wherein the library of candidate delivery vehicles of step (c) includes multiple different nanoparticle formulations.
   26. The method of any one of 1-25, wherein one or more properties selected from group consisting of: ligand density on the delivery vehicle, molecular weight of polymers, anchor length, and ratio of carrier molecules; are modulated for an additional round of screening.
   27. The method of any one of 1-26, wherein said selecting of step (f) comprises selecting from 1-15 top-performing targeting ligands.
   28. The method of any one of 1-27, wherein an automated system:
performs steps (a) and (b) using differential expression data provided by a user;
robotically synthesizes the library of candidate delivery vehicles; and
performs said evaluating of step (e).
   29. The method of any one of 1-28, wherein the library of step (c) includes one or more delivery vehicles with heteromultivalent targeting ligands.
   30. The method of any one of 1-29, wherein a recursive optimization algorithm is used to drive one or more additional rounds of screening.
   31. The method of any one of 1-30, wherein a flow-based peptide synthesis system is used to assemble the candidate targeting ligands.
   32. The method of any one of 1-31, wherein predictions of formulation performance metrics in a given screening iteration are algorithmically compared with analytically-derived performance metrics to refine computational methods of performance metrics prediction from formulation parameters in a subsequent screening.
   33. The method of any one of 1-32, wherein one or more of the selected targeting ligands are coupled to synthetically-made DNA, PNA or RNA in order to create a patient-specific therapeutic response.
   34. A method of generating a diagnostically-responsive delivery vehicle that can be used to target cells, tissues, or organs of an individual, the method comprising:
(a) obtaining molecular diagnostic information from the individual;
(b) identifying one or more cell surface targets based on (a); and
(c) producing a delivery vehicle comprising one or more targeting ligands that target the one or more cell surface targets.
   35. The method of 34, wherein the molecular diagnostic information of step (a) comprises at least: nucleic acid sequencing data, microarray expression data, or proteomics expression data obtained from the individual.
   36. The method of 34 or 35, wherein the delivery vehicle comprises the one or more targeting ligands fused to an anchoring domain.
   37. The method of 36, wherein the anchoring domain is a charged polymer polypeptide domain that interacts with a protein and/or nucleic acid payload.
   38. The method of 34 or 35, wherein the delivery vehicle is a nanoparticle.
   39. The method of 38, wherein the nanoparticle comprises a core that comprises: an anionic polymer composition; a cationic polymer composition; a cationic polypeptide composition; and a protein and/or nucleic acid payload.
   40. The method of 38, wherein the nanoparticle comprises a core comprising cross-linked polymers.
   41. The method of 38, wherein the nanoparticle comprises a SH residue for coupling to a substrate.
   42. The method of 38, wherein the nanoparticle comprises a solid core particle.
   43. The method of any one of 34-42, wherein the delivery vehicle is a lipid-based delivery system that comprises a protein and/or nucleic acid payload.
   44. The method of any one of 37-43, wherein the protein and/or nucleic acid payload comprises one or more gene editing tools.
   45. The method of any one of 34-44, wherein step (b) comprises calculating a cell, tissue, or organ selectivity index for candidate cell surface targets in order to identify the 3-50 highest expressed surface proteins of the cell, tissue, or organ of interest.
   46. The method of any one of 34-44, wherein step (b) comprises calculating a cell, tissue, or organ selectivity index for candidate cell surface targets in order to identify the 3-10 highest and uniquely expressed surface proteins of the cell, tissue, or organ of interest.
   47. The method of any one of 34-46, wherein said producing of step (c) comprises:
(i) generating a list of candidate targeting ligands;
(ii) producing a library of candidate delivery vehicles, wherein each candidate delivery vehicle displays one or more of the candidate targeting ligands from the list generated in step (i);
(iii) contacting the identified one or more cell surface targets of step (b) with the library of candidate delivery vehicles of step (ii);
(iv) evaluating effectiveness of the candidate targeting ligands to target the one or more cell surface targets based on results of said contacting; and
(v) selecting one or more candidate targeting ligands based on said evaluating to be the one or more targeting ligands of step (c).
   48. The method of 47, wherein step (i) comprises evaluating crystal structures of the one or more cell surface targets to derive protein-ligand or protein-protein interaction information for the one or more cell surface targets.
   49. The method of 48, wherein the protein-ligand or protein-protein interaction information is used to identify a secondary structure scaffold and the candidate targeting ligands are designed to conform to said secondary structure scaffold.
   50. The method of any one of 47-49, wherein the list of candidate targeting ligands of step (i) includes one or more ligand types selected from the group consisting of: an antibody, a scFv, a nanobody, a chemically synthesized peptide, and a nucleic acid aptamer.
   51. The method of any one of 47-49, wherein the list of candidate targeting ligands of step (i) includes one or more ligands identified by phage display screening.
   52. The method of any one of 47-51, wherein, after step (v), at least one of the selected targeting ligands is subject to mutagenesis to produce a second library of delivery vehicles that display one or more variants of the at least one of the selected targeting ligands, and a second round of contacting, evaluating, and selecting is performed.
   53. The method of any one of 47-51, further comprising, after step (v), generating candidate delivery vehicle formulations for a second round of screening using the one or more selected targeting ligands of step (v).
   54. The method of 53, wherein, after step (v), a machine learning approach is used to approximate an objective function and to generate said candidate delivery vehicle formulations for the second round of screening.
   55. The method of any one of 47-54, wherein:
said contacting of step (iii) comprises contacting cells that express said one or more surface targets with the library of candidate delivery vehicles,
the candidate delivery vehicles of step (ii) comprise a detectable payload, and
said evaluating of step (iv) comprises measuring the detectable payload present in said cells after said contacting.
   56. The method of 55, wherein the candidate delivery vehicles of step (ii) comprise a targeting ligand fused to the detectable payload.
   57. The method of 55 or 56, wherein said evaluating of step (iv) comprises an evaluation of physicochemical data of the candidate delivery vehicles in addition to biological data from said contacting.
   58. The method of 57, wherein said biological data includes one or more of the following parameters: percent of cells that take-up the payload, rate of payload uptake, cell subtype specificity/selectivity, increased cell division activity, gene expression, and cell toxicity.
   59. The method of any one of 47-58, wherein the candidate delivery vehicles of step (ii) comprise a targeting ligand fused to an anchoring domain.
   60. The method of 59, wherein the anchoring domain is a charged polymer polypeptide domain that interacts with a detectable payload.
   61. The method of any one of 47-60, wherein the candidate delivery vehicles of step (ii) are nanoparticles.
   62. The method of 61, wherein the nanoparticles comprise a core comprising: an anionic polymer composition, a cationic polymer composition, a cationic polypeptide composition, and a detectable payload.
   63. The method of 62, wherein the detectable payload is a nucleic and/or protein payload.
   64. The method of any one of 61-63, wherein, after step (v), aggregate databases of nanopar parameters and their characterized performance metrics are used to predict new candidate formulation performance metrics, whereby these predictions are used to inform and/or guide modifications and refinements to candidate formulations.
   65. The method of any one of 47-64, wherein the library of candidate delivery vehicles of step (ii) includes multiple different nanoparticle formulations.
   66. The method of any one of 47-65, wherein one or more properties selected from group consisting of: ligand density on the delivery vehicle, molecular weight of polymers, anchor length, and ratio of carrier molecules; are modulated for an additional round of screening.
   67. The method of any one of 47-66, wherein said selecting of step (v) comprises selecting from 34-15 top-performing targeting ligands.
   68. The method of any one of 47-67, wherein an automated system:
performs step (b) using the molecular diagnostic information of step (a);
robotically synthesizes the library of candidate delivery vehicles; and
performs said evaluating of step (iv).
   69. The method of any one of 47-68, wherein the library of step (ii) includes one or more delivery vehicles with heteromultivalent targeting ligands.
   70. The method of any one of 47-69, wherein a recursive optimization algorithm is used to drive one or more additional rounds of screening.
   71. The method of any one of 47-70, wherein a flow-based peptide synthesis system is used to assemble the candidate targeting ligands.
   72. The method of any one of 47-71, wherein predictions of formulation performance metrics in a given screening iteration are algorithmically compared with analytically-derived performance metrics to refine computational methods of performance metrics prediction from formulation parameters in a subsequent screening.
   73. The method of any one of 34-72, wherein the method comprises administering the delivery vehicle produced in step (c) to the individual, wherein the individual has a disorder or disease and the delivery vehicle comprises a protein and/or nucleic acid payload for treating the disorder or disease. 74. A method of treating an individual who has a disease, the method comprising:
   administering a delivery vehicle to an individual who has a disease, wherein the delivery vehicle
delivers a payload composition to a diseased cell of the individual, wherein the payload composition comprises one or both of
(1) an affinity marker or a nucleic acid encoding the affinity marker, wherein the affinity marker is a surface protein that is thereby displayed and/or expressed on the surface of the diseased cell; and
(2) a secreted protein or a nucleic acid encoding the secreted protein, wherein the secreted protein activates the individual's immune system.
   75. The method of 74, wherein the individual has cancer and the diseased cell is a cancer cell.
   76. The method of 74, wherein the individual has a solid tumor and the diseased cell is a cell of the solid tumor.
   77. The method of any one of 74-76, wherein the affinity arker is a chimeric fusion protein that comprises a membrane anchor fused to an extracellular protein domain that is recognized by and activates the individual's immune system.
   78. The method of any one of 74-76, wherein the affinity marker is a heterologous protein that the diseased cell did not express prior to said administering.
   79. The method of any one of 74-76, wherein the affinity marker is a protein that the diseased cell expresses prior to said administering, but expresses at a higher level after said administering.
   80. The method of any one of 74-79, wherein the payload composition comprises donor DNA, and a nucleotide sequence of the donor DNA integrates into the diseased cell's genome.
   81. The method of any one of 74-79, wherein the payload composition comprises a double stranded DNA gene expression cassette that does not integrate into the diseased cell's genome, wherein the double stranded DNA gene expression cassette comprises a nucleotide sequence of interest operably linked to a promoter.
   82. The method of 81, wherein the promoter is selected by evaluating gene expression of diseased cells of the individual.
   83. The method of any one of 74-79, wherein the payload composition comprises an mRNA.
   84. The method of any one of 74-83, wherein the delivery vehicle is non-viral.
   85. The method of any one of 74-83, wherein the delivery vehicle is a nanoparticle.
   86. The method of 85, wherein the nanoparticle comprises a core comprising an anionic polymer composition, a cationic polymer composition, and a cationic polypeptide composition.
   87. The method of 86, wherein said anionic polymer composition comprises an anionic polymer selected from poly(glutamic acid) and poly(aspartic acid).
   88. The method of 86 or 87, wherein said cationic polymer composition comprises a cationic polymer selected from poly(arginine), poly(lysine), poly(histidine), poly(ornithine), and poly(citrulline).
   89. The method of any one of 86-88, wherein nanoparticle further comprises a sheddable layer encapsulating the core.
   90. The method of 89, wherein the sheddable layer is an anionic coat or a cationic coat.
   91. The method of 89 or 90, wherein the sheddable layer comprises one or more components selected from: silica, a peptoid, a polycysteine, calcium, calcium oxide, hydroxyapatite, calcium phosphate, calcium sulfate, manganese, manganese oxide, manganese phosphate, manganese sulfate, magnesium, magnesium oxide, magnesium phosphate, magnesium sulfate, iron, iron oxide, iron phosphate, iron sulfate, and an anionic polymer.
   92. The method of any one of 89-91, wherein the nanoparticle further comprises a surface coat surrounding the sheddable layer.
   93. The method of 92, wherein the surface coat comprises a cationic or anionic anchoring domain that interacts electrostatically with the sheddable layer.
   94. The method of 92 or 93, wherein the surface coat comprises one or more targeting ligands.
   95. The method of 94, wherein at least one of said one or more targeting ligands targets a surface protein of the diseased cell, wherein the surface protein was identified by evaluating diseased cells of the individual.
   96. The method of any one of 92-95, wherein the surface coat comprises one or more stealth motifs.
   97. The method of 96, wherein said one or more stealth motifs comprise one or more components selected from: hyaluronan, polyethylene glycol, a polysialic acid functionalized peptide, a sialic acid functionalized peptide, a glycopeptide, a glycan-modified polymer backbone, and a neuraminic acid functionalized peptide.
   98. The method of any one of 74-97, wherein the payload composition comprises the affinity marker or the nucleic acid encoding the affinity marker.
   99. The method of 98, wherein the affinity marker is bound by an endogenous T cell receptor, which elicits a cytotoxic response.
   100. The method of 98, wherein the affinity marker engages a direct signaling cascade.
   101. The method of 98, wherein the method further comprises introducing an engineered T-cell into the individual, wherein the engineered T-cell expresses a receptor that binds to the affinity marker.
   102 The method of 101, wherein the T-cell is a CAR T-cell.
   103. The method of 98, wherein the method further comprises introducing an engineered natural killer cell (NK cell) into the individual, wherein the engineered NK cell expresses a receptor that binds to the affinity marker.
   104. The method of 98, wherein the method further comprises introducing an engineered immune cell into the individual, wherein the engineered immune cell expresses a receptor that binds to the affinity marker.
   105, The method of any one of 74-104, wherein the payload composition comprises the secreted protein or the nucleic acid encoding the secreted protein.
   106. The method of 105, wherein the secreted protein is a cytokine and is selected from: IL-2, IL-7, IL-12, IL-15, IL-21, and IFN-gamma.
   107. The method of any one of 74-106, wherein the delivery vehicle is a targeting ligand conjugated to a charged polymer domain, wherein the targeting ligand provides for targeted binding to a cell surface protein, and wherein the charged polymer domain is condensed with and/or is interacting electrostatically with the payload composition.
   108. The method of 107, wherein the delivery vehicle further comprises an anionic polymer interacting with the payload composition and the charged polymer domain.
   109. The method of any one of 74-106, wherein the delivery vehicle is a targeting ligand directly conjugated to a substrate
   110. The method of 109, wherein the substrate is selected from: a solid core, an interlayer, an end of a PEG group, a linear polymer, and a branched polymer.

What is claimed is:

1. A method of generating a targeting ligand that can be used to target cells, tissues, or organs of interest, the method comprising:
(g) identifying one or more cell surface targets for targeting a cell, tissue, or organ of interest;
(h) generating a list of candidate targeting ligands;
(i) producing a library of candidate delivery vehicles, wherein each candidate delivery vehicle displays one or more of the candidate targeting ligands from the list generated in step (b);
(j) contacting the identified one or more cell surface targets of step (a) with the library of candidate delivery vehicles of step (c);
(k) evaluating effectiveness of the candidate targeting ligands to target the one or more cell surface targets based on results of said contacting; and
(l) selecting one or more targeting ligands based on said evaluating.

2. The method of claim 1, wherein step (a) comprises calculating a cell, tissue, or organ selectivity index for candidate cell surface targets in order to identify the 3-50 highest expressed surface proteins of the cell, tissue, or organ of interest.

3. The method of claim 1, wherein step (a) comprises calculating a cell, tissue, or organ selectivity index for candidate cell surface targets in order to identify the 3-10 highest and uniquely expressed surface proteins of the cell, tissue, or organ of interest.

4. The method of claim 1, wherein step (b) comprises evaluating crystal structures of the one or more cell surface targets to derive protein-ligand or protein-protein interaction information for the one or more cell surface targets.

5. The method of claim 4, wherein the protein-ligand or protein-protein interaction information is used to identify a secondary structure scaffold and the candidate targeting ligands are designed to conform to said secondary structure scaffold.

6. The method of claim 1, wherein the list of candidate targeting ligands of step (b) includes one or more ligand types selected from the group consisting of: an antibody, a scFv, a nanobody, a chemically synthesized peptide, and a nucleic acid aptamer.

7. The method of claim 1, wherein the list of candidate targeting ligands of step (b) includes one or more ligands identified by phage display or random peptide library screening.

8. The method of claim 1, wherein, after step (0, at least one of the selected targeting ligands is subject to mutagenesis to produce a second library of delivery vehicles that display one or more variants of the at least one of the selected targeting ligands, and a second round of contacting, evaluating, and selecting is performed.

9. The method of claim 1, further comprising, after step (0, generating candidate delivery vehicle formulations for a second round of screening using the one or more selected targeting ligands of step (f).

10. The method of claim 9, wherein, after step (0, a machine learning approach is used to approximate an objective function and to generate said candidate delivery vehicle formulations for the second round of screening.

11. The method of any claim 1, wherein:
(i) said contacting of step (d) comprises contacting cells that express said one or more surface targets with the library of candidate delivery vehicles,
(ii) the candidate delivery vehicles of step (c) comprise a detectable payload, and
(iii) said evaluating of step (e) comprises measuring the detectable payload present in said cells after said contacting.

12. The method of claim 11, wherein the candidate delivery vehicles of step (c) comprise a targeting ligand fused to the detectable payload.

13. The method of claim 11, wherein said evaluating of step (e) comprises an evaluation of physicochemical data of the candidate delivery vehicles in addition to biological data from said contacting.

14. The method of claim 13, wherein said biological data includes one or more of the following parameters: percent of cells that take-up the payload, rate of payload uptake, cell subtype specificity/selectivity, increased cell division activity, gene expression, and cell toxicity.

15. The method of claim 1, wherein the candidate delivery vehicles of step (c) comprise a targeting ligand fused to an anchoring domain.

16. The method of claim 15, wherein the anchoring domain is a charged polymer polypeptide domain that interacts with a detectable payload.

17. The method of claim 1, wherein the candidate delivery vehicles of step (c) are nanoparticles.

18. The method of claim 17, wherein the nanoparticles comprise a core comprising: an anionic polymer composition, a cationic polymer composition, a cationic polypeptide composition, and a detectable payload.

19. The method of claim 17, wherein the nanoparticles comprise a core comprising cross-linked polymers.

20. The method of claim 17, wherein the nanoparticles comprise a SH residue for coupling to a substrate.
